(19) United States
(12) Patent Application Publication
(10) Pub. No.: US 2026/0288002 A1
(43) Pub. Date: Sep. 24, 2026

(54) SALT, ACID GENERATOR, RESIN, RESIST COMPOSITION, AND METHOD FOR PRODUCING RESIST PATTERN

(71) Applicant: Sumitomo Chemical Company, Limited, Tokyo (JP)

(72) Inventors: Yuji KITA, Osaka (JP); Koji ICHIKAWA, Osaka (JP)

(73) Assignee: Sumitomo Chemical Company, Limited, Tokyo (JP)

(21) Appl. No.: 19/555,206

(22) Filed: Mar. 3, 2026

(30) Foreign Application Priority Data
Mar. 6, 2025 (JP) 2025-035192

(51) Int. Cl. G03F 7/004; C07C 381/12; C08F 22/24; G03F 7/038; G03F 7/039; G03F 7/16

(52) U.S. Cl. CPC G03F 7/0045; C07C 381/12; C08F 22/24; G03F 7/0382; G03F 7/0388; G03F 7/0397; G03F 7/168

(57) ABSTRACT

An acid generator containing a salt represented by a formula (I) or a structural unit represented by a formula (IP)

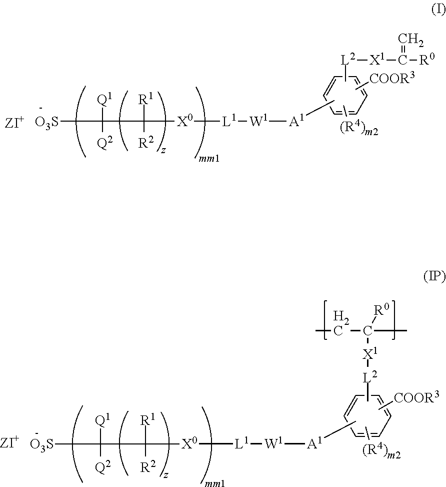

### BACKGROUND OF THE INVENTION

### Field of the Invention

The present invention relates to a salt for an acid generator used for fine processing of semiconductors, an acid generator including the salt, a resin, a resist composition, and a method for producing a resist pattern.

### Description of the Related Art

WO-A-2024-190386 describes a resin including a structural unit derived from a salt represented by the following formula, and a resist composition containing the resin.

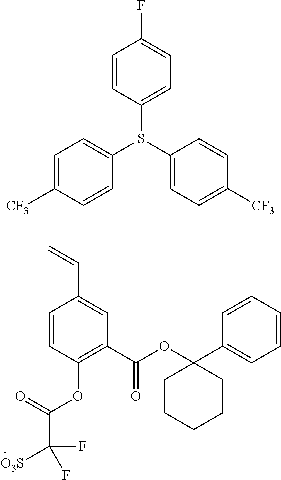

WO-A-2024-014462 describes a resin including a structural unit derived from a salt represented by the following formula, and a resist composition containing the resin.

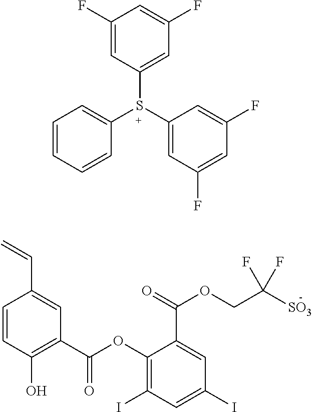

### SUMMARY OF THE INVENTION

An object of the present invention is to provide a resist composition for forming a resist pattern having more favorable CD uniformity (CDU) than a resist pattern formed from a resist composition including a resin containing a structural unit derived from a salt.

The present invention includes the following inventions.

[1] An acid generator containing a salt represented by a formula (I) or a structural unit represented by a formula (IP), in which

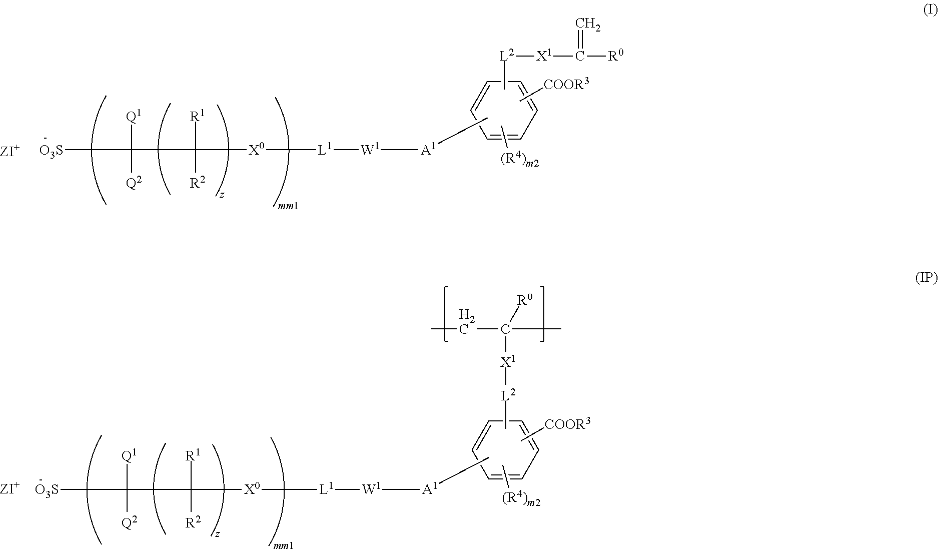

in the formula (I) and the formula (IP),
Q¹, Q², R¹, and R² each independently represent a hydrogen atom, a fluorine atom, a perfluoroalkyl group having 1 to 6 carbon atoms or an alkyl group having 1 to 6 carbon atoms,
z represents an integer of 0 to 6, and when z is 2 or more, a plurality of R¹ and R² may be the same or different from each other,
X⁰ represents a single bond, or a group formed by —O—, —CO—, —S—, —SO—, —SO₂—, —NR⁷—, or a combination thereof,
mm1 represents 0 or 1,
L¹ and L² each independently represent a single bond or a hydrocarbon group having 1 to 28 carbon atoms and optionally having a substituent, and —CH₂— included in the hydrocarbon group may be substituted with —O—, —S—, —SO₂—, —SO—, —NR⁷—, or —CO—,
R⁷ represents a hydrogen atom or an alkyl group having 1 to 6 carbon atoms,
W¹ represents a single bond or a cyclic hydrocarbon group having 3 to 36 carbon atoms and optionally having a substituent, and —CH₂— included in the cyclic hydrocarbon group may be substituted with —O—, —S—, —SO₂—, —SO—, —NR⁷—, or —CO—,
A¹ represents a single bond or a hydrocarbon group having 1 to 20 carbon atoms, the hydrocarbon group may have a substituent, and —CH₂— included in the hydrocarbon group may be substituted with —O—, —S—, —SO₂—, —SO—, —NR⁷—, or —CO—,
R³ represents an acid stable hydrocarbon group optionally having a substituent,
R⁴ represents a halogen atom, a cyano group, a nitro group, or a hydrocarbon group having 1 to 18 carbon atoms, the hydrocarbon group may have a substituent, and —CH₂— included in the hydrocarbon group may be substituted with —O—, —S—, —SO₂—, —SO—, —NR⁷—, or —CO—,
m2 represents an integer of 0 to 3, and when m2 is 2 or more, a plurality of R⁴ may be the same or different from each other,
X¹ represents a single bond, or —O—, —CO—, —S—, —SO—, —SO₂—, —NR⁷—, a phenylene group optionally having a substituent, or a group obtained by combining these groups,
R⁰ represents a hydrogen atom, a halogen atom, or an alkyl group having 1 to 6 carbon atoms and optionally having a halogen atom, and
ZI⁺ represents an organic cation.
   [2] The acid generator according to [1], in which
A¹ is a single bond, ***-X⁰¹-L⁰¹-, or ***-L⁰¹-X⁰¹-,
X⁰¹ represents —O—, —CO—, —S—, —SO—, —NR⁷—, or —SO₂—,
L⁰¹ represents a single bond or a hydrocarbon group having 1 to 18 carbon atoms, in which the hydrocarbon group may have a substituent, and —CH₂— included in the hydrocarbon group may be substituted with —O—, —CO—, —S—, —SO—, —NR⁷—, or —SO₂—, and
*** represents a binding site with W¹.
   [3] The acid generator according to [2], in which
X⁰¹ is —O— or —CO—, and
L⁰¹ is a single bond or an alkanediyl group having 1 to 6 carbon atoms, in which —CH₂— included in the alkanediyl group may be substituted with —O— or —CO—.
   [4] The acid generator according to any one of [1] to [3], in which W¹ is a single bond or an aromatic hydrocarbon group having 6 to 10 carbon atoms and optionally having a substituent.
   [5] The acid generator according to any one of [1] to [4], in which an acid stable hydrocarbon group optionally having a substituent of R³ is a group represented by a formula (10a-1) or a group represented by a formula (10a-2), in which

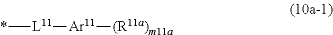

in the formula (10a-1),
L¹¹ represents a single bond or an alkanediyl group having 1 to 6 carbon atoms,
Ar¹¹ represents an aromatic hydrocarbon group having 6 to 18 carbon atoms,
R^{11a} represents a halogen atom or an alkyl group having 1 to 12 carbon atoms and optionally having a halogen atom, and —CH₂— included in the alkyl group may be substituted with —O— or —CO—,
m11a represents an integer of 0 to 8, when m11a is 2 or more, a plurality of R^{11a} may be the same or different from each other, and
* represents a binding site, and

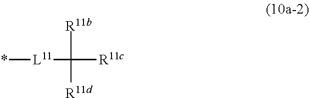

in the formula (10a-2),
L¹¹ represents a single bond or an alkanediyl group having 1 to 6 carbon atoms, for R^{11b}, R^{11c}, and R^{11d},
R^{11b} and R^{11c} each independently represent a hydrogen atom or a hydrocarbon group having 1 to 18 carbon atoms, and R^{11d} is a hydrogen atom, or
R^{11b} and R^{11c} are bonded to each other to form an alicyclic hydrocarbon group having 3 to 36 carbon atoms and optionally having a halogen atom or optionally having an alkyl group having 1 to 12 carbon atoms and optionally having a halogen atom, in which —CH₂— included in the alkyl group may be substituted with —O— or —CO—, and R^{11d} is a hydrogen atom or
R^{11b}, R^{11c}, and R^{11d} are bonded to each other to form an alicyclic hydrocarbon group having 3 to 36 carbon atoms and optionally having a halogen atom or an alkyl group having 1 to 12 carbon atoms and optionally having a halogen atom, in which —CH₂— included in the alkyl group may be substituted with —O— or —CO—, and
* represents a binding site.
   [6] The acid generator according to any one of [1] to [5], in which an acid stable hydrocarbon group optionally having a substituent of R³ is a group including a cyclic hydrocarbon group having 3 to 18 carbon atoms, the group having at least one selected from the group consisting of a halogen atom and a haloalkyl group having 1 to 12 carbon atoms, in which the cyclic hydrocarbon group may have a substituent other than a halogen atom and a haloalkyl group.
   [7] The acid generator according to any one of [1] to [6], in which m2 is an integer of 1 or more, and R⁴ is at least one selected from the group consisting of a halogen atom and a hydrocarbon group having 1 to 18 carbon atoms (—CH₂— included in the hydrocarbon group may be substituted with —O— or —CO—).
   [8] The acid generator according to any one of [1] to [7], in which X¹ is a single bond or a group represented by any one of formulas (X¹-1) to (X¹-10),

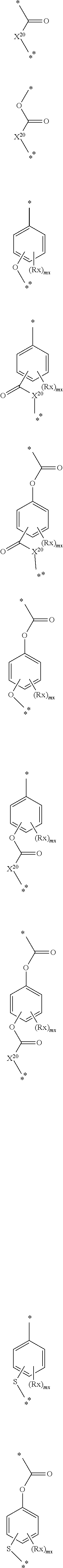

in the formula (X¹-1) to the formula (X¹-10),
* and ** are binding sites, and * represents a binding site to carbon atoms to which R⁰ is bonded,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms,
mx represents an integer of 0 to 4, when mx is 2 or more, a plurality of Rx may be the same or different from each other,
X²⁰ represents —O— or —NR⁷—, and
R⁷ represents a hydrogen atom or an alkyl group having 1 to 6 carbon atoms.
   [9]A resist composition containing the acid generator according to any one of [1] to [8].
   [10] The resist composition according to [9], in which
an acid generator is a salt represented by a formula (I), and
the resist composition further includes a resin including a structural unit (a1) having an acid-labile group.
   [11] The resist composition according to [9], in which
an acid generator is a resin including a structural unit represented by a formula (IP), and
the resin further includes the structural unit (a1) having an acid-labile group.
   [12] The resist composition according to [11], further including a salt represented by the formula (I).
   [13] The resist composition according to any one of [9] to [12], in which the structural unit (a1) having an acid-labile group includes at least one selected from the group consisting of a structural unit represented by a formula (a1-0), a structural unit represented by a formula (a1-1), a structural unit represented by a formula (a1-2), a structural unit represented by a formula (a1-4), a structural unit represented by a formula (a1-5), and a structural unit represented by a formula (a1-6),

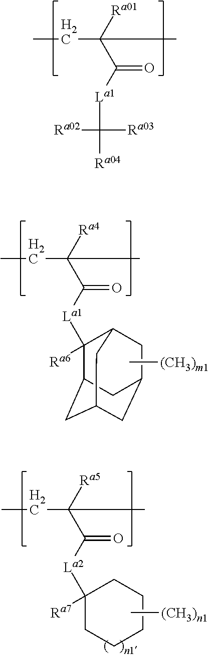

in the formula (a1-0), the formula (a1-1), and the formula (a1-2),
L^{a01}, L^{a1}, and L^{a2} each independently represent —O— or *—O—(CH₂)ₖ₁—CO—O—, k1 represents an integer of 1 to 7, and * represents a binding site to —CO—,
R^{a01}, R^{a4}, and R^{a5} each independently represent a hydrogen atom, a halogen atom or an alkyl group having 1 to 6 carbon atoms and optionally having a halogen atom,
R^{a02}, R^{a03}, and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alkenyl group, the alicycic hydrocarbon group, and the aromatic hydrocarbon group may have a halogen atom,
m1′ represents an integer of 0 to 14,
n1 represents an integer of 0 to 14, and
n1′ represents an integer of 0 to 3,

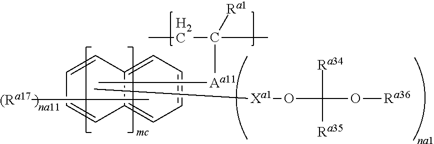

in the formula (a1-4),
R^{a1} represents a hydrogen atom, a halogen atom, or an alkyl group having 1 to 6 carbon atoms and optionally having a halogen atom,
R^{a17} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group,
A^{a11} represents a single bond or an alkanediyl group having 1 to 12 carbon atoms, and —CH₂— included in the alkanediyl group may be substituted with —O—, —CO—, or —NR^{a18}—,
R^{a18} represents a hydrogen atom or an alkyl group having 1 to 6 carbon atoms,
X^{a1} represents a single bond or a carbonyl group,
R^{a34} and R^{a35} each independently represent a hydrogen atom or a hydrocarbon group having 1 to 12 carbon atoms, R^{a33} represents a hydrocarbon group having 1 to 20 carbon atoms, or R^{a34} represents a hydrogen atom or a hydrocarbon group having 1 to 12 carbon atoms, R^{a35} and R^{a36} may be bonded to each other to form a divalent heterocyclic group having 3 to 20 carbon atoms together with —C—O— to which they are bonded, and —CH₂— included in the hydrocarbon group and the divalent heterocyclic group may be substituted with —O— or —S—,
na1 represents an integer of 1 to 5, and when na1 is 2 or more, a plurality of groups in parentheses may be the same or different from each other,
na11 represents an integer of 0 to 4, and when na11 is 2 or more, a plurality of R^{a17} may be the same as or different from each other, and
mc represents an integer of 0 to 2,

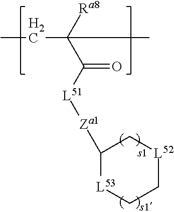

in the formula (a1-5),
R^{a8} represents an alkyl group having 1 to 6 carbon atoms and optionally having a halogen atom, a hydrogen atom, or a halogen atom,
Z^{a1} represents a single bond or *—(CH₂)ₕ₃—CO-L⁵⁴-, h3 represents an integer of 1 to 4, and * represents a binding site to L⁵¹,
L⁵¹, L⁵², L⁵³, and L⁵⁴ each independently represent —O— or —S—,
s1 represents an integer of 1 to 3, and
s1′ represents an integer of 0 to 3,

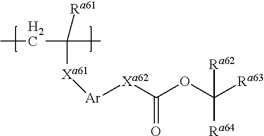

in the formula (a1-6),
R^{a61} represents a hydrogen atom, a halogen atom, or an alkyl group having 1 to 6 carbon atoms and optionally having a halogen atom,
R^{a62}, R^{a63}, and R^{a64} each independently represent an alkyl group having 1 to 6 carbon atoms or a cyclic hydrocarbon group having 3 to 18 carbon atoms and optionally having a substituent, or R^{a62} and R^{a63} are bonded to each other to form a ring having 3 to 20 carbon atoms together with the carbon atom to which they are bonded, and R^{a64} represents an alkyl group having 1 to 6 carbon atoms or a cyclic hydrocarbon group having 3 to 18 carbon atoms and optionally having a substituent,
X^{a61} represents a single bond, —CO—O—* or —CO—NR^{a65-}*, * represents a binding site to Ar, and R^{a65} represents a hydrogen atom or an alkyl group having 1 to 6 carbon atoms,
X^{a62} represents a single bond, *—O-L^{a61-} or *—CO—O-L^{a62-}, * represents a binding site to Ar, and L^{a61} and L^{a62} each independently represent an alkanediyl group having 1 to 4 carbon atoms, and
Ar represents an aromatic hydrocarbon group having 6 to 20 carbon atoms and optionally having a substituent.
   [14] The resist composition according to any one of [9] to [13], including a resin containing a structural unit represented by a formula (a2-A),

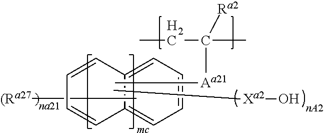

in the formula (a2-A),
R^{a2} represents a hydrogen atom, a halogen atom, or an alkyl group having 1 to 6 carbon atoms and optionally having a halogen atom,
R^{a27} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group,
A^{a21} represents a single bond or an alkanediyl group having 1 to 12 carbon atoms, and —CH₂— included in the alkanediyl group may be substituted with —O—, —CO—, or —NR^{a28-},
R^{a28} represents a hydrogen atom or an alkyl group having 1 to 6 carbon atoms,
X^{a2} represents a single bond or a carbonyl group,
nA2 represents an integer of 1 to 5, and when nA2 is 2 or more, a plurality of groups in parentheses may be the same as or different from each other,
na21 represents an integer of 0 to 4, and when na21 is 2 or more, a plurality of R^{a27} may be the same as or different from each other, and
mc represents an integer of 0 to 2,
   [15] The resist composition according to any one of [9] to [14], further including a salt generating an acid having an acidity lower than that of an acid generated from the acid generator.
   [16]A method for producing a resist pattern, the method including:
(1) applying the resist composition according to any one of [9] to [15] onto a substrate;
(2) drying the applied resist composition to form a composition layer; (3) exposing the composition layer;
(4) heating the exposed composition layer; and
(5) developing the heated composition layer.
   [17]A salt represented by the formula (I) described above.
   [18] The acid generator according to [1], in which
A¹ is a single bond, ***-X⁰¹-L⁰¹-, or ***-L⁰¹-X⁰¹-,
X⁰¹ represents —O—, —CO—, —S—, —SO—, —NR⁷—, or —SO₂—,
L⁰¹ represents a single bond or a hydrocarbon group having 1 to 18 carbon atoms, in which the hydrocarbon group may have a substituent, and —CH₂— included in the hydrocarbon group may be substituted with —O—, —CO—, —S—, —SO—, —NR⁷—, or —SO₂—, and
*** represents a binding site to W¹.

The acid generator according to [2], in which
X⁰¹ is —O— or —CO—, and
L⁰¹ is a single bond or an alkanediyl group having 1 to 6 carbon atoms, in which —CH₂— included in the alkanediyl group may be substituted with —O— or —CO—.
   [20] The salt according to any one of [17] to [19], in which W¹ is a single bond or an aromatic hydrocarbon group having 6 to 10 carbon atoms and optionally having a substituent.
   [21] The salt according to any one of [17] to [20], in which the acid-stable hydrocarbon group optionally having a substituent for R³ is a group represented by the formula (10a-1) or a group represented by the formula (10a-2).
   [22] The salt according to any one of [17] to [21], in which an acid stable hydrocarbon group optionally having a substituent of R³ is a group including a cyclic hydrocarbon group having 3 to 18 carbon atoms, the group having at least one selected from the group consisting of a halogen atom and a haloalkyl group having 1 to 12 carbon atoms, in which the cyclic hydrocarbon group may have a substituent other than a halogen atom and a haloalkyl group.

The salt according to any one of [17] to [22], in which m2 is an integer of 1 or more, and R⁴ is at least one selected from the group consisting of a halogen atom and a hydrocarbon group having 1 to 18 carbon atoms, in which —CH₂— included in the hydrocarbon group may be substituted with —O— or —CO—.

The salt according to any one of [17] to [23], in which X¹ is a single bond or a group represented by any one of the formulas (X¹-1) to (X¹-10) described above.

A resin including the structural unit represented by the above-described formula (IP).

It is possible to produce a resist pattern with satisfactory CD uniformity (CDU) by using a resist composition of the present invention.

### DETAILED DESCRIPTION OF THE PREFERRED EMBODIMENTS

As used herein, “(meth)acrylic monomer” means “at least one of acrylic monomer and methacrylic monomer”. Notations such as “(meth)acrylate” and “(meth)acrylic acid” mean the same thing. In groups described herein, regarding groups capable of having both a linear structure and a branched structure, they may have either the linear or branched structure. When —CH₂— included in the hydrocarbon group or the like is substituted with —O—, —S—, —CO—, —SO—, —NR^{x}— (X is any symbol), or —SO₂—, the same examples shall apply for each group, and the number of carbon atoms before substitution is taken as the number of carbon atoms of the hydrocarbon group or the like. “Combined group” means a group in which two or more exemplified groups are bonded, and valences of those groups may be appropriately varied by bonding forms. “Derived” or “Induced” means that a polymerizable C═C bond included in the molecule becomes a —C—C— group (single bond) by polymerization. When stereoisomers exist, all stereoisomers are included. Hydrogen atoms at any position and any number of hydrogen atoms included in each group may be sometimes substituted with a binding site depending on the number of substituents or the like. The number of carbon atoms in the substituents is not included in the number of carbon atoms in the group to be substituted. The “acid-labile group” means a group in which a leaving group is eliminated by contact with an acid (for example, trifluoromethanesulfonic acid, etc.), thus forming a hydrophilic group (for example, a hydroxy group or a carboxy group). The “base-labile group” means a group in which a leaving group is eliminated upon contact with a base (for example, trimethylamine, tetramethylammonium hydroxide etc.), thus forming a hydrophilic group (for example, a carboxy group or a hydroxy group).

As used herein, “solid content of the resist composition” means the total amount of components in which the below-described solvent (E) is removed from the total amount of the resist composition.

### [Salt Represented by Formula (I)]

The present invention relates to a salt represented by the formula (I) (hereinafter may be referred to as “salt (I)”).

Of the salt (I), the side having negative charge may be referred to as “anion (I)”, and the side having positive charge may be referred to as “cation (I)”.

### (Anion (I))

Examples of the perfluoroalkyl group as for Q¹, Q², R¹ and R² in the formula (I) include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, and a perfluorohexyl group. The number of carbon atoms of the perfluoroalkyl group is preferably 1 to 4, and more preferably 1 to 3.

Examples of the alkyl group as for Q¹, Q², R¹ and R² include a methyl group, an ethyl group, a propyl group, an isopropyl group, a butyl group, a sec-butyl group, a tert-butyl group, a pentyl group, and a hexyl group. The number of carbon atoms of the alkyl group is preferably 1 to 4, and more preferably 1 to 3.

Preferably, at least one of Q¹ and Q² includes a fluorine atom or a perfluoroalkyl group, more preferably at least one is a fluorine atom or a perfluoroalkyl group, still more preferably each independently is a trifluoromethyl group or a fluorine atom, and yet more preferably both of them are fluorine atoms.

Preferably, R¹ and R² are each independently a hydrogen atom, a fluorine atom, a methyl group or a trifluoromethyl group, more preferably a hydrogen atom or a fluorine atom, and still more preferably a hydrogen atom.

z is preferably an integer of 0 to 4, more preferably an integer of 0 to 3, still more preferably an integer of 0 to 2, and yet more preferably 0 or 1.

Examples of the alkyl group as R⁷ include the same alkyl groups as those listed for Q¹. R⁷ is preferably a hydrogen atom or an alkyl group having 1 to 4 carbon atoms, more preferably a hydrogen atom or an alkyl group having 1 to 3 carbon atoms, still more preferably a hydrogen atom, an ethyl group, or a methyl group, and yet more preferably a hydrogen atom or a methyl group.

X⁰ is preferably a single bond, or —O—, —CO—, —NR⁷—, or a combination thereof, more preferably a single bond, *—CO—O—, *—O—CO—, *—O—, *—O—CO—O—NR⁷—, *—CO—NR⁷—, *—NR⁷—CO—, *—NR⁷—CO—O—, or *—O—CO—NR⁷—, still more preferably *—CO—O—, *—O—CO—O—, or *—O—CO—, yet more preferably *—CO—O— or *—O—CO—O—, and further preferably *—CO—O—. * represents a binding site to C(R¹)(R²) or C(Q¹)(Q²).

The hydrocarbon group in L¹ and L² may be either a saturated or unsaturated group, and examples thereof include a linear or branched chain hydrocarbon group such as an alkanediyl group, a monocyclic or polycyclic (a spiro ring, a fused ring, a bridged ring, or the like) alicyclic hydrocarbon group, and an aromatic hydrocarbon group, and may include a group obtained by combining two or more of these groups (a hydrocarbon group formed from, for example, an alicyclic hydrocarbon group or an aromatic hydrocarbon group and a chain hydrocarbon group). The chain hydrocarbon group and the alicyclic hydrocarbon group are also referred to as an aliphatic hydrocarbon group, and the alicyclic hydrocarbon group and the aromatic hydrocarbon group are also referred to as a cyclic hydrocarbon group. The number of carbon atoms of the hydrocarbon group is preferably 1 to 24, more preferably 1 to 20, still more preferably 1 to 18, and yet more preferably 1 to 12.

Examples of the alkanediyl group include linear alkanediyl groups such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group, and
branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The end of the branched alkanediyl group may be an alkyl group such as a methyl group.

The number of carbon atoms in the chain hydrocarbon group is preferably 1 to 18, more preferably 1 to 12, still more preferably 1 to 10, yet more preferably 1 to 8, further preferably 1 to 6, and further more preferably 1 to 4.

Examples of the monocyclic or polycyclic divalent alicyclic hydrocarbon group include the following groups. The binding site can be any position.

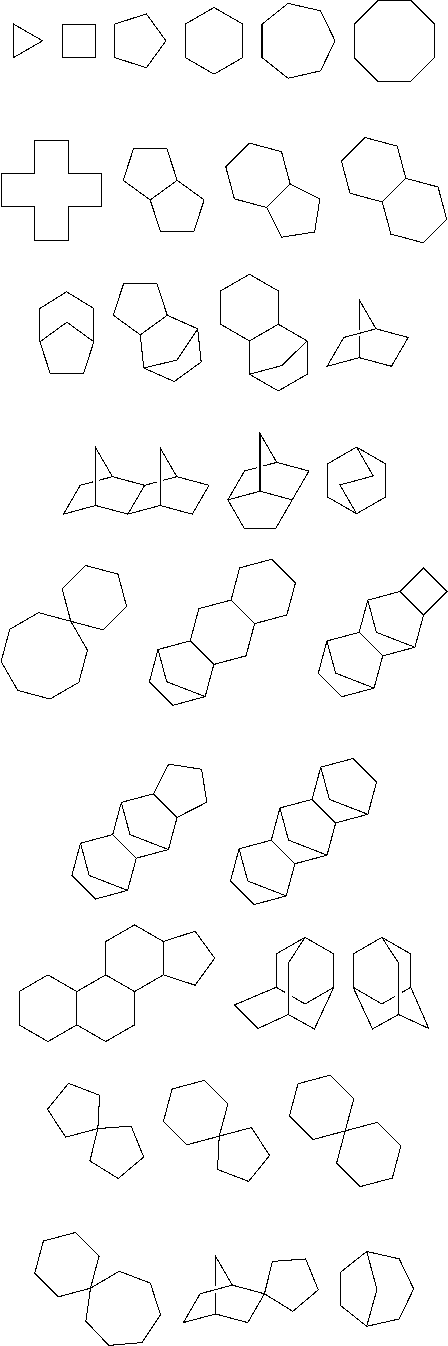

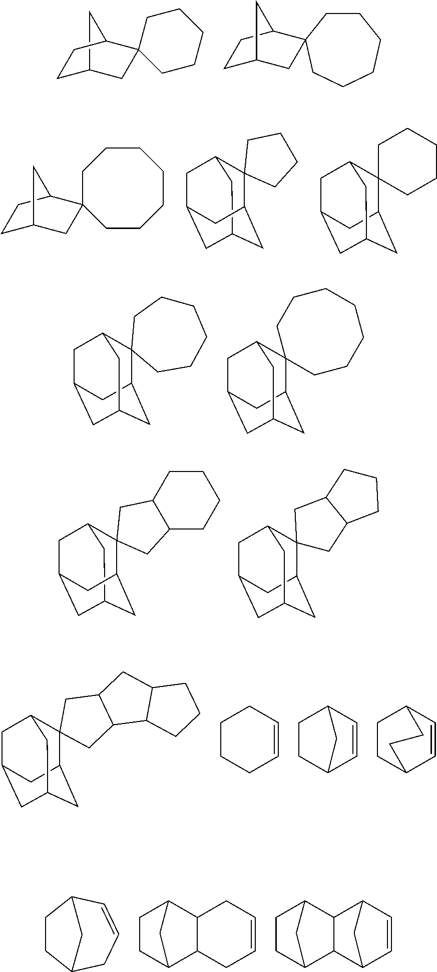

Specific examples of the alicyclic hydrocarbon groups include monocyclic divalent alicyclic hydrocarbon groups which are monocyclic cycloalkanediyl groups, such as a cyclobutane-1,3-diyl group, a cyclopentane-1,3-diyl group, a cyclohexane-1,4-diyl group and a cyclooctane-1,5-diyl group, and
polycyclic divalent alicyclic hydrocarbon groups that are polycyclic cycloalkanediyl groups such as a norbornane-1,4-diyl group, a norbornane-2,5-diyl group, an adamantane-1,5-diyl group, an adamantane-2,6-diyl group, a spirocyclohexane-1,2′-cyclopentyl group, cycloalkyl groups such as a spiroadamantane-2,3′-cyclopentyl group, and spirocycles having a cycloalkyl group bonded to a norbornyl group or an adamantyl group via a spiro bond. The number of carbon atoms of the alicyclic hydrocarbon group is preferably 3 to 18, more preferably 3 to 16, and still more preferably 3 to 12.

Examples of the aromatic hydrocarbon groups include aromatic hydrocarbon groups, for example, arylene groups such as a phenanthrylene group, a naphthylene group, an anthrylene group, a biphenylene group, and a phenanthrene group. The number of carbon atoms of the aromatic hydrocarbon group is preferably 6 to 18, more preferably 6 to 14, and still more preferably 6 to 10.

Examples of the group obtained by combining two or more groups include a group obtained by combining an alicyclic hydrocarbon group with a chain hydrocarbon group, a group obtained by combining an aromatic hydrocarbon group with a chain hydrocarbon group, a group obtained by combining an alicyclic hydrocarbon group with an aromatic hydrocarbon group, and a group obtained by combining an alicyclic hydrocarbon group, an aromatic hydrocarbon group and a chain hydrocarbon group. In combination, two or more of alicyclic hydrocarbon groups, aromatic hydrocarbon groups, and chain hydrocarbon groups may be respectively combined. In addition, any group may be bonded to X⁰ or X¹.

Examples of the group obtained by combining an alicyclic hydrocarbon group with a chain hydrocarbon group include a -divalent alicyclic hydrocarbon group-alkanediyl group-, an -alkanediyl group-divalent alicyclic hydrocarbon group-alkanediyl group-, and an -alkanediyl group-divalent alicyclic hydrocarbon group-.

Examples of the group obtained by combining an aromatic hydrocarbon group with a chain hydrocarbon group include a -divalent aromatic hydrocarbon group-alkanediyl group-, an -alkanediyl group-divalent aromatic hydrocarbon group-alkanediyl group-, and an -alkanediyl group-divalent aromatic hydrocarbon group-.

Examples of the group obtained by combining an alicyclic hydrocarbon group with an aromatic hydrocarbon group include a -aromatic hydrocarbon group-alicyclic hydrocarbon group-, a -alicyclic hydrocarbon group-aromatic hydrocarbon group-, and a -alicyclic hydrocarbon group-aromatic hydrocarbon group-alicyclic hydrocarbon group.

—CH₂— included in the hydrocarbon group having 1 to 28 carbon atoms of L¹ and L² may be substituted with —O—, —S—, —SO₂—, —SO—, —NR⁷—, or —CO—.

When —CH₂— included in the hydrocarbon group having 1 to 28 carbon atoms of L¹ and L² is substituted with —O—, —S—, —SO₂—, —SO—, —NR⁷—, or —CO—, the number of carbon atoms before substitution is defined as the number of carbon atoms of the hydrocarbon group.

Examples of the hydrocarbon groups in which —CH₂— is substituted with —O—, —S—, —SO₂—, —SO—, —NR⁷—, or —CO— include a hydroxy group (a methyl group in which —CH₂— is substituted with —O—), a carboxy group (an ethyl group in which —CH₂—CH₂— is substituted with —O—CO—), a thiol group (a methyl group in which —CH₂— is substituted with —S—), an amino group (a methyl group in which —CH₂— is substituted with —NR⁷—, where R⁷ is a hydrogen atom), an oxy group (a group in which —CH₂— in a methylene group is substituted with —O—), a carbonyl group (a group in which —CH₂— in a methylene group is substituted with —CO—), a
thio group (a group in which —CH₂— in a methylene group is substituted with —S—), a sulfonyl group (a group in which —CH₂— in a methylene group is substituted with —SO₂—),
an ester group (a group in which —CH₂—CH₂— in an ethylene group is substituted with —CO—O—), a carbonate group (a group in which —CH₂—CH₂— CH₂— in a propylene group is substituted with —O—CO—O—), a peptide group (a group in which —CH₂—CH₂— in an ethylene group is substituted with —CO— NR⁷—, where R⁷ is a hydrogen atom), an alkoxy group (a group in which —CH₂— at any position in an alkyl group is substituted with —O—), an alkylthio group (a group in which —CH₂— at any position in an alkyl group is substituted with —S—), an alkylsulfonyl group (a group in which —CH₂— at any position in an alkyl group is substituted with —SO₂—), an alkylamino group (a group in which —CH₂— at any position in an alkyl group is substituted with —NR⁷—, where R⁷ is a hydrogen atom), an alkylpeptide group (a group in which —CH₂—CH₂— at any position in an alkyl group is substituted with —CO— NR⁷—), where R⁷ is a hydrogen atom), an alkoxycarbonyl group (a group in which —CH₂—CH₂— at any position within the alkyl group is substituted with —O—CO—), an alkylcarbonyl group (a group in which —CH₂— at any position in the alkyl group is substituted with —CO—), an alkylcarbonyloxy group (a group in which —CH₂—CH₂— at any position in the alkyl group is substituted with —CO—O—), an alkoxycarbonyloxy group (a group in which —CH₂—CH₂—CH₂— at any position in the alkyl group is substituted with —O—CO—O—), an alkoxyalkoxy group (a group in which two —CH₂— at any position in the alkyl group are substituted with —O—), an alkoxyalkyl group (a group in which —CH₂— at any position in the alkyl group is substituted with —O—), a hydroxyalkyl group (a group in which a —CH₂— in a methyl group at any position in an alkyl group is substituted with —O—), an alkanediyloxy group (a group in which a —CH₂— in an alkanediyl group at any position is substituted with —O—), an alkanediyloxycarbonyl group (a group in which a —CH₂—CH₂— in an alkanediyl group at any position is substituted with —O—CO—), an alkanediylcarbonyl group (a group in which a —CH₂— in an alkanediyl group at any position is substituted with —CO—), an alkanediylcarbonyloxy group (a group in which a —CH₂—CH₂— in an alkanediyl group at any position is substituted with —CO—O—), an alkanediyloxycarbonyloxy group (a group in which a —CH₂—CH₂— in an alkanediyl group at any position is substituted with —O—CO—O—), an alkanediylsulfonyl group (a group in which —CH₂— included in an alkanediyl group at any position is substituted with —SO₂—), an alkanediylthio group (a group in which —CH₂— included in an alkanediyl group at any position is substituted with —S—), an alkanediylamino group (a group in which —CH₂— in an alkanediyl group at any position is substituted with —NR⁷—, where R^{T} is an alkyl group having 1 to 6 carbon atoms), an alkanediylpeptide group (a group in which —CH₂—CH₂— included in an alkanediyl group at any position is substituted with —CO—NR⁷—, where R^{T} is an alkyl group having 1 to 6 carbon atoms), a cycloalkoxy group, a cycloalkylalkoxy group, an aromatic hydrocarbon group-carbonyloxy group, an aromatic hydrocarbon group-carbonyl group, an aromatic hydrocarbon group-oxy group, and combinations of two or more of these groups.

Examples of the alkoxy group include alkoxy groups having 1 to 27 carbon atoms, for example, a methoxy group, an ethoxy group, a propoxy group, a butoxy group, a pentyloxy group, a hexyloxy group, an octyloxy group, a 2-ethylhexyloxy group, a nonyloxy group, a decyloxy group, and an undecyloxy group. The number of carbon atoms of the alkoxy group is preferably 1 to 11, more preferably 1 to 6, still more preferably 1 to 4, and yet more preferably 1 to 3.

Examples of the alkylthio group include alkylthio groups having 1 to 27 carbon atoms, for example, a methylthio group, an ethylthio group, a propylthio group, a butylthio group, a pentylthio group, a hexylthio group, an octylthio group, a 2-ethylhexylthio group, a nonylthio group, a decylthio group, and an undecylthio group. The number of carbon atoms of the alkylthio group is preferably 1 to 11, more preferably 1 to 6, still more preferably 1 to 4, and yet more preferably 1 to 3.

Examples of the alkylsulfonyl group include alkylsulfonyl groups having 1 to 27 carbon atoms, for example, a methylsulfonyl group, an ethylsulfonyl group, a propylsulfonyl group, a butylsulfonyl group, a pentylsulfonyl group, a hexylsulfonyl group, an octylsulfonyl group, a 2-ethylhexylsulfonyl group, a nonylsulfonyl group, a decylsulfonyl group, and an undecylsulfonyl group. The number of carbon atoms of the alkylsulfonyl group is preferably 1 to 11, more preferably 1 to 6, still more preferably 1 to 4, and yet more preferably 1 to 3.

Examples of the alkylamino group include alkylamino groups having 1 to 27 carbon atoms, for example, a methylamino group, an ethylamino group, a dimethylamino group, a propylamino group, a butylamino group, a pentylamino group, a pentylamino group, and a hexylamino group. The number of carbon atoms of the alkylamino group is preferably 1 to 11, more preferably 1 to 6, still more preferably 1 to 4, and yet more preferably 1 to 3.

Examples of the alkylpeptide group include alkylpeptide groups having 1 to 27 carbon atoms, for example, a methylpeptide group, an ethylpeptide group, and a propylpeptide group. The number of carbon atoms of the alkylpeptide group is preferably 1 to 11, more preferably 1 to 6, still more preferably 1 to 4, and yet more preferably 1 to 3.

Examples of the alkoxycarbonyl group include alkoxycarbonyl groups having 2 to 27 carbon atoms, for example, a methoxycarbonyl group, an ethoxycarbonyl group, and a butoxycarbonyl group. Examples of the alkylcarbonyl group include alkylcarbonyl groups having 2 to 28 carbon atoms, for example, an acetyl group, a propionyl group and a butyryl group. Examples of the alkylcarbonyloxy group include alkylcarbonyloxy groups having 2 to 27 carbon atoms, for example, an acetyloxy group, a propionyloxy group, and a butyryloxy group. Examples of the alkoxycarbonyloxy group include alkoxycarbonyloxy groups having 2 to 26 carbon atoms, for example, a butoxycarbonyloxy group. Examples of the alkoxyalkoxy group include alkoxyalkoxy groups having 2 to 26 carbon atoms, for example, a methoxymethoxy group, a methoxyethoxy group, an ethoxymethoxy group, and an ethoxyethoxy group. The number of carbon atoms of the alkoxycarbonyl group is preferably 2 to 11, more preferably 2 to 6, still more preferably 2 to 4, and yet more preferably 2 or 3. The number of carbon atoms of the alkylcarbonyl group is preferably 2 to 12, more preferably 2 to 6, still more preferably 2 to 4, and yet more preferably 2 or 3. The number of carbon atoms of the alkylcarbonyloxy group is preferably 2 to 11, more preferably 2 to 6, still more preferably 2 to 4, and yet more preferably 2 or 3. The number of carbon atoms of the alkoxycarbonyloxy group is preferably 2 to 10, more preferably 2 to 6, still more preferably 2 to 4, and yet more preferably 2 or 3. The number of carbon atoms of the alkoxyalkoxy group is preferably 2 to 10, more preferably 2 to 6, still more preferably 2 to 4, and yet more preferably 2 or 3.

Examples of the alkoxyalkyl groups include alkoxyalkyl groups having 2 to 27 carbon atoms, for example, a methoxymethyl group, a methoxyethyl group, an ethoxymethyl group,
an ethoxyethyl group, an ethoxypropyl group, an ethoxybutyl group,
a propoxymethyl group, a propoxyethyl group, an isopropoxymethyl group, a propoxypropyl group,
a butoxymethyl group, a sec-butoxymethyl group, and a tert-butoxymethyl group. The number of carbon atoms of the alkoxyalkyl group is preferably 2 to 11, more preferably 2 to 6, still more preferably 2 to 4, and yet more preferably 2 or 3.

Examples of the hydroxyalkyl groups include hydroxyalkyl groups having 1 to 27 carbon atoms, for example, a hydroxymethyl group, a dihydroxymethyl group, a hydroxyethyl group, a dihydroxyethyl group, a hydroxypropyl group, a hydroxyisopropyl group, a hydroxybutyl group, and a hydroxysec-butyl group. The number of carbon atoms of the hydroxyalkyl group is preferably 1 to 11, more preferably 1 to 6, still more preferably 1 to 4, and yet more preferably 1 to 3.

Examples of the alkanediyloxy group include alkanediyloxy groups having 1 to 27 carbon atoms, for example, a methyleneoxy group, an ethyleneoxy group, a propanediyloxy group, a butanediyloxy group, and a pentanediyloxy group. The number of carbon atoms of the alkanediyloxy group is preferably 1 to 11, more preferably 1 to 6, still more preferably 1 to 4, and yet more preferably 1 to 3.

Examples of the alkanediyloxycarbonyl group include alkanediyloxycarbonyl groups having 2 to 27 carbon atoms, for example, a methyleneoxycarbonyl group, an ethyleneoxycarbonyl group, a propanediyloxycarbonyl group, and a butanediyloxycarbonyl group. Examples of the alkanediylcarbonyl group include alkanediylcarbonyl groups having 2 to 28 carbon atoms, for example, a methylenecarbonyl group, an ethylenecarbonyl group, a propanediylcarbonyl group, a butanediylcarbonyl group, and a pentanediylcarbonyl group. Examples of the alkanediylcarbonyloxy group include alkanediylcarbonyloxy groups having 2 to 27 carbon atoms, for example, a methylenecarbonyloxy group, an ethylenecarbonyloxy group, a propanediylcarbonyloxy group, and a butanediylcarbonyloxy group. Examples of the alkanediyloxycarbonyloxy group include alkoxycarbonyloxy groups having 2 to 26 carbon atoms, for example, a butoxycarbonyloxy group. The number of carbon atoms of the alkanediyloxycarbonyl group is preferably 2 to 11, more preferably 2 to 6, still more preferably 2 to 4, and yet more preferably 2 or 3. The number of carbon atoms of the alkanediylcarbonyl group is preferably 2 to 12, more preferably 2 to 6, still more preferably 2 to 4, and yet more preferably 2 or 3. The number of carbon atoms of the alkanediylcarbonyloxy group is preferably 2 to 11, more preferably 2 to 6, still more preferably 2 to 4, and yet more preferably 2 or 3. The number of carbon atoms of the alkanediyloxycarbonyloxy group is preferably 2 to 10, more preferably 2 to 6, still more preferably 2 to 4, and yet more preferably 2 or 3.

Examples of the alkanediylsulfonyl group include alkanediylsulfonyl groups having 1 to 27 carbon atoms, for example, a methylenesulfonyl group, an ethylenesulfonyl group, and a propylenesulfonyl group. The number of carbon atoms of the alkanediylsulfonyl group is preferably 1 to 11, more preferably 1 to 6, still more preferably 1 to 4, and yet more preferably 1 to 3.

Examples of the alkanediylthio group include alkanediylthio groups having 1 to 27 carbon atoms, for example, a methylenethio group, an ethylenethio group, and a propylenethio group. The number of carbon atoms of the alkanediylthio group is preferably 1 to 11, more preferably 1 to 6, still more preferably 1 to 4, and yet more preferably 1 to 3.

Examples of the alkanediylamino group include alkanediylamino groups having 1 to 27 carbon atoms, for example, a methyleneamino group, an ethyleneamino group, a dimethyleneamino group, a propyleneamino group, a butyleneamino group, a pentyleneamino group, and a hexyleneamino group. The number of carbon atoms of the alkanediylamino group is preferably 1 to 11, more preferably 1 to 6, still more preferably 1 to 4, and yet more preferably 1 to 3.

Examples of the alkanediylpeptide group include alkanediylpeptide groups having 1 to 27 carbon atoms, for example, a methylenepeptide group, an ethylenepeptide group, and a propylenepeptide group. The number of carbon atoms of the alkanediylpeptide group is preferably 1 to 11, more preferably 1 to 6, still more preferably 1 to 4, and yet more preferably 1 to 3.

Examples of the cycloalkoxy group include cycloalkoxy groups having 3 to 27 carbon atoms, for example, a cyclohexyloxy group. Examples of the cycloalkylalkoxy group include cycloalkylalkoxy groups having 4 to 27 carbon atoms, for example, a cyclohexylmethoxy group. Examples of the aromatic hydrocarbon group-carbonyloxy group include aromatic hydrocarbon group-carbonyloxy group having 7 to 27 carbon atoms, for example, a benzoyloxy group. Examples of the aromatic hydrocarbon group-carbonyl group include aromatic hydrocarbon group-carbonyl groups having 7 to 28 carbon atoms, for example, a benzoyl group. Examples of the aromatic hydrocarbon group-oxy group include aromatic hydrocarbon group-oxy groups having 6 to 27 carbon atoms, for example, a phenyloxy group.

In addition, examples of the group in which —CH₂— included in an alicyclic hydrocarbon group, an aromatic hydrocarbon group, or a group obtained by combining these groups is substituted with —O—, —S—, —CO—, —SO—, —NR⁷—, or —SO₂— include the following groups. —O— or —CO— of the group represented below may be substituted with —S—, —SO—, —NR⁷— or —SO₂—. The binding site can be any position.

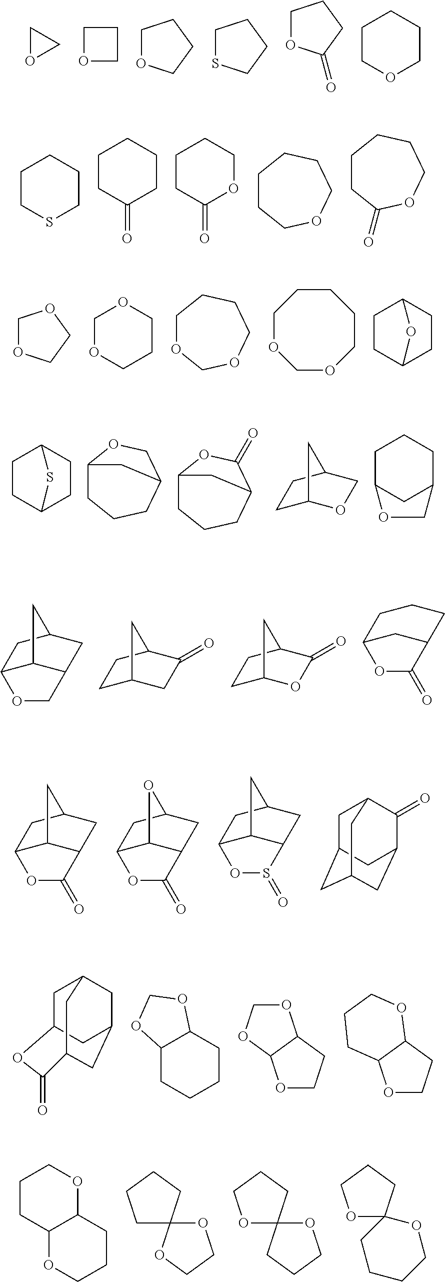

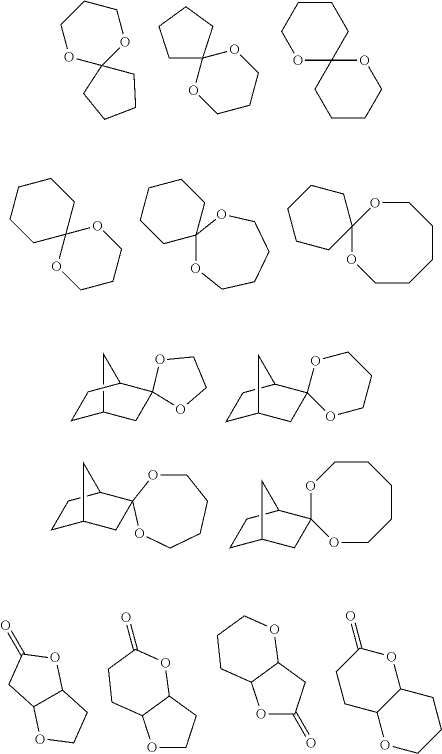

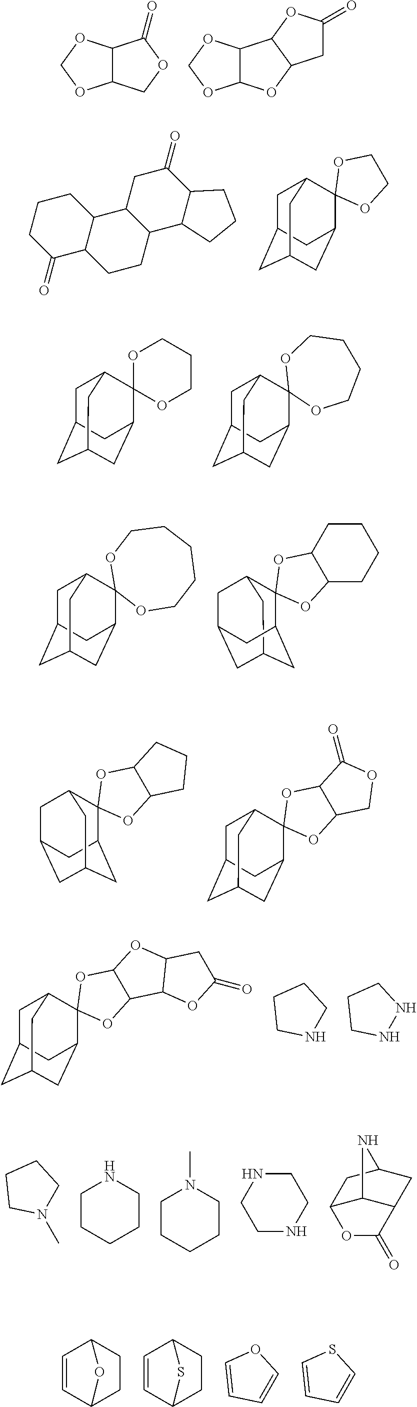

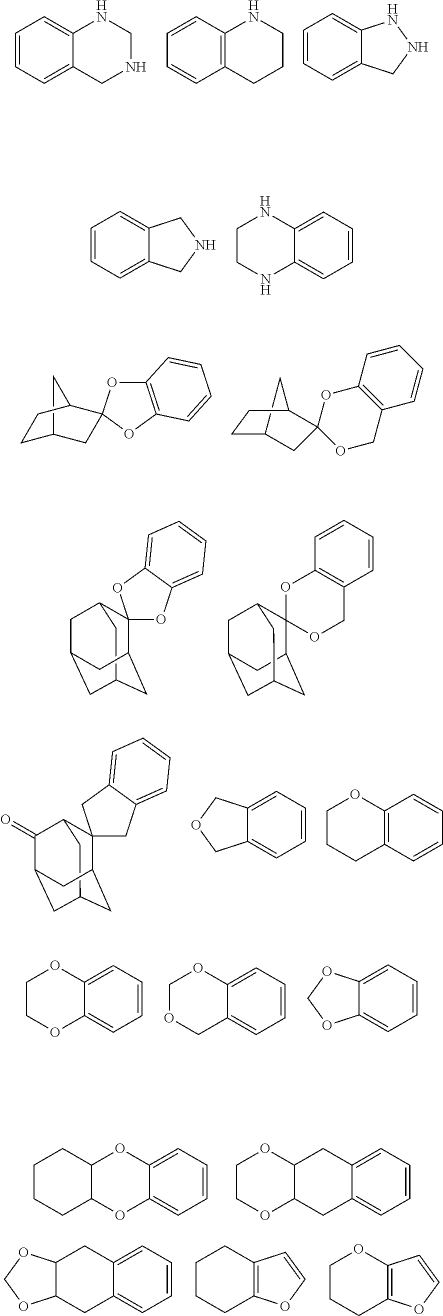

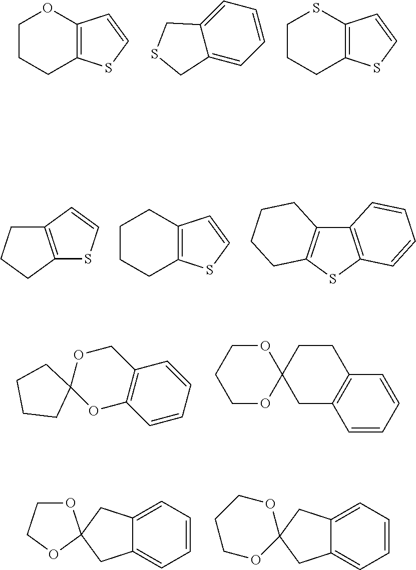

Examples of the substituent which may be possessed by L¹ and L² include a halogen atom, a cyano group, and a nitro group. —CH₂— in the chain hydrocarbon group, such as the alkyl group, of L¹ and L² is substituted with —O—, —S—, —CO—, —SO—, —NR⁷—, or —SO₂—, whereby L¹ and L² can substantially have a substituent, such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group, or an alkylpeptide group. When L¹ and L² are groups obtained by combining an alicyclic hydrocarbon group or an aromatic hydrocarbon group with a chain hydrocarbon group such as an alkyl group, the chain hydrocarbon group such as an alkyl group can serve as the substituent of the alicyclic hydrocarbon group or the aromatic hydrocarbon group.

Examples of the halogen atom include a fluorine atom, a chlorine atom, a bromine atom, and an iodine atom.

Examples of L¹ include: a single bond, a chain hydrocarbon group having 1 to 18 carbon atoms and optionally having a substituent (—CH₂— in the chain hydrocarbon group may be substituted with —O— or —CO—), a cyclic hydrocarbon group having 3 to 18 carbon atoms and optionally having a substituent (—CH₂— in the cyclic hydrocarbon group may be substituted with —O—, —S—, —SO₂—, —SO—, —NR⁷—, or —CO—), or a group combining a chain hydrocarbon group having 1 to 18 carbon atoms and optionally having a substituent with a cyclic hydrocarbon group having 3 to 18 carbon atoms and optionally having a substituent (—CH₂— in the chain hydrocarbon group may be substituted with —O— or —CO—, and —CH₂— in the cyclic hydrocarbon group may be substituted with —O—, —S—, —SO₂—, —SO—, —NR⁷—, or —CO—); preferably, a single bond, a chain hydrocarbon group having 1 to 12 carbon atoms and optionally having a substituent (—CH₂— in the chain hydrocarbon group may be substituted with —O— or —CO—), a cyclic hydrocarbon group having 3 to 18 carbon atoms and optionally having a substituent (—CH₂— in the cyclic hydrocarbon group may be substituted with —O—, —S—, —SO₂—, —SO—, —NR⁷—, or —CO—), or a group combining a chain hydrocarbon group having 1 to 10 carbon atoms and optionally having a substituent with a cyclic hydrocarbon group having 3 to 18 carbon atoms and optionally having a substituent (—CH₂— in the chain hydrocarbon group may be substituted with —O— or —CO—, and —CH₂— in the cyclic hydrocarbon group may be substituted with —O—, —S—, —SO₂—, —SO—, —NR⁷—, or —CO—); more preferably, a single bond, an alkanediyl group having 1 to 10 carbon atoms (—CH₂— in the alkanediyl group may be substituted with —O— or —CO—), or a group combining an alkanediyl group having 1 to 6 carbon atoms with a cyclic hydrocarbon group having 3 to 12 carbon atoms and optionally having a substituent (—CH₂— in the alkanediyl group and the cyclic hydrocarbon group may be substituted with —O— or —CO—); more preferably, a single bond, an alkanediyl group having 1 to 8 carbon atoms (—CH₂— in the alkanediyl group may be substituted with —O— or —CO—), a group combining an alkanediyl group having 1 to 4 carbon atoms with an alicyclic hydrocarbon group having 3 to 12 carbon atoms and optionally having a substituent (—CH₂— in the alkanediyl group and the alicyclic hydrocarbon group may be substituted with —O— or —CO—), or a group combining an alkanediyl group having 1 to 4 carbon atoms with an aromatic hydrocarbon group having 6 to 10 carbon atoms and optionally having a substituent (—CH₂— in the alkanediyl group may be substituted with —O— or —CO—); and still more preferably, a single bond, an alkanediyl group having 1 to 8 carbon atoms, or a group combining an alkanediyl group having 1 to 4 carbon atoms with an aromatic hydrocarbon group having 6 to 10 carbon atoms (—CH₂— in the alkanediyl group may be substituted with —O— or —CO—, and the aromatic hydrocarbon group may have a fluorine atom, an iodine atom, a hydroxy group, or an alkoxy group having 1 to 3 carbon atoms).

Examples of L² include: a single bond, a chain hydrocarbon group having 1 to 18 carbon atoms and optionally having a substituent (—CH₂— in the chain hydrocarbon group may be substituted with —O— or —CO—), a cyclic hydrocarbon group having 3 to 18 carbon atoms and optionally having a substituent (—CH₂— in the cyclic hydrocarbon group may be substituted with —O—, —S—, —SO₂—, —SO—, —NR⁷—, or —CO—), or a group combining a chain hydrocarbon group having 1 to 18 carbon atoms and optionally having a substituent with a cyclic hydrocarbon group having 3 to 18 carbon atoms and optionally having a substituent (—CH₂— in the chain hydrocarbon group may be substituted with —O— or —CO—, and —CH₂— in the cyclic hydrocarbon group may be substituted with —O—, —S—, —SO₂—, —SO—, —NR⁷—, or —CO—); preferably, a single bond, a chain hydrocarbon group having 1 to 12 carbon atoms and optionally having a substituent (—CH₂— in the chain hydrocarbon group may be substituted with —O— or —CO—), a cyclic hydrocarbon group having 3 to 18 carbon atoms and optionally having a substituent (—CH₂— in the cyclic hydrocarbon group may be substituted with —O—, —S—, —SO₂—, —SO—, —NR⁷—, or —CO—), or a group combining a chain hydrocarbon group having 1 to 10 carbon atoms and optionally having a substituent with a cyclic hydrocarbon group having 3 to 18 carbon atoms and optionally having a substituent (—CH₂— in the chain hydrocarbon group may be substituted with —O— or —CO—, and —CH₂— in the cyclic hydrocarbon group may be substituted with —O—, —S—, —SO₂—, —SO—, —NR⁷—, or —CO—); more preferably, a single bond, an alkanediyl group having 1 to 10 carbon atoms (—CH₂— in the alkanediyl group may be substituted with —O— or —CO—), or a group combining an alkanediyl group having 1 to 6 carbon atoms with a cyclic hydrocarbon group having 3 to 12 carbon atoms and optionally having a substituent (—CH₂— in the alkanediyl group and the cyclic hydrocarbon group may be substituted with —O— or —CO—); more preferably, a single bond, an alkanediyl group having 1 to 8 carbon atoms (—CH₂— in the alkanediyl group may be substituted with —O— or —CO—), a group combining an alkanediyl group having 1 to 4 carbon atoms with an alicyclic hydrocarbon group having 3 to 12 carbon atoms and optionally having a substituent (—CH₂— in the alkanediyl group and the alicyclic hydrocarbon group may be substituted with —O— or —CO—), or a group combining an alkanediyl group having 1 to 4 carbon atoms with an aromatic hydrocarbon group having 6 to 10 carbon atoms and optionally having a substituent (—CH₂— in the alkanediyl group may be substituted with —O— or —CO—); and still more preferably, a single bond, an alkanediyl group having 1 to 4 carbon atoms, or a group combining an alkanediyl group having 1 to 4 carbon atoms with an aromatic hydrocarbon group having 6 to 10 carbon atoms (—CH₂— in the alkanediyl group may be substituted with —O— or —CO—, and the aromatic hydrocarbon group may have a fluorine atom, an iodine atom, a hydroxy group, or an alkoxy group having 1 to 3 carbon atoms).

The cyclic hydrocarbon group having 3 to 36 carbon atoms hereinafter, may be referred to as a “ring W¹”) of W¹ may be either saturated or unsaturated, and may be either monocyclic or polycyclic (a spiro ring, a fused ring, a bridged ring, or the like is included). Examples of the cyclic hydrocarbon group include an alicyclic hydrocarbon group, an aromatic hydrocarbon group, and a group obtained by combining these groups. The carbon atoms included in the alicyclic hydrocarbon group may form a double bond between two adjacent carbon atoms. The number of carbon atoms in the cyclic hydrocarbon group is preferably 3 to 30, more preferably 3 to 24, and still more preferably 3 to 20.

Examples of the alicyclic hydrocarbon group include the same groups as those exemplified for L¹ and L². Specific examples of the monocyclic alicyclic hydrocarbon group include monocyclic cycloalkanediyl groups such as a cyclopropanediyl group, a cyclobutanediyl group, a cyclopentanediyl group, a cyclohexanediyl group, a cycloheptanediyl group, a cyclooctanediyl group, and a cyclodecanediyl group. Examples of the polycyclic alicyclic hydrocarbon group include polycyclic cycloalkanediyl groups such as a decahydronaphthanediyl group, an adamantanediyl group, a norbornanediyl group, a norbornenediyl group, and a bicyclo[3.3.0]octanediyl group.

The number of carbon atoms of the alicyclic hydrocarbon group is preferably 3 to 24, more preferably 3 to 18, still more preferably 3 to 16, and yet more preferably 3 to 12.

Examples of the aromatic hydrocarbon group include aromatic hydrocarbon groups such as arylene groups such as a phenylene group, a naphthylene group, an anthrylene group, a biphenylene group, a phenanthrylene group, an indenylene group, a fluorenylene group, and a diphenylmethane group.

The number of carbon atoms of the aromatic hydrocarbon group is preferably 6 to 24, more preferably 6 to 18, still more preferably 6 to 14, and yet more preferably 6 to 10.

—CH₂— included in the cyclic hydrocarbon group may be substituted with —O—, —S—, —CO—, —SO—, —NR⁷—, or —SO₂—, and examples of such a substituted group include the same groups as those exemplified in the present specification as long as the number of carbon atoms is acceptable.

The cyclic hydrocarbon group is preferably a cyclic hydrocarbon group having 3 to 18 carbon atoms (a hydrogen atom may be substituted with a substituent, and —CH₂— may be substituted with —O—, —S—, —CO—, —SO—, —NR⁷—, or —SO₂—), more preferably, an alicyclic hydrocarbon group having 3 to 18 carbon atoms or an aromatic hydrocarbon group having 6 to 18 carbon atoms (a hydrogen atom may be substituted with a substituent, and —CH₂— may be substituted with —O—, —S—, —CO—, —SO—, —NR⁷—, or —SO₂—), and more preferably, a group represented by any of formulas (w1-1) to (w1-33).

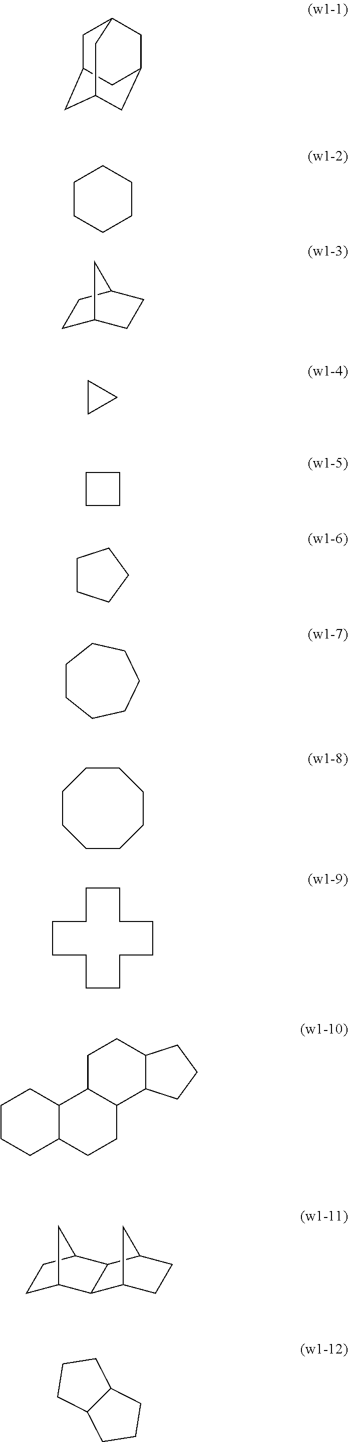

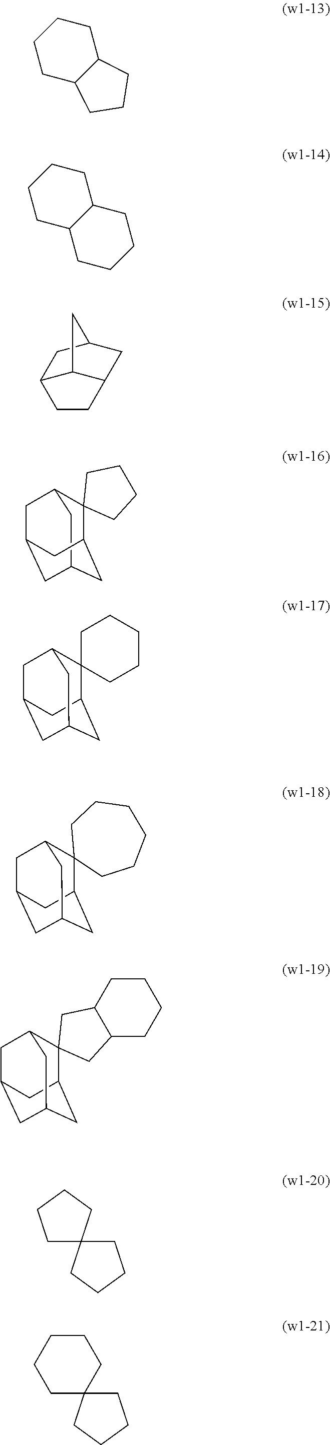

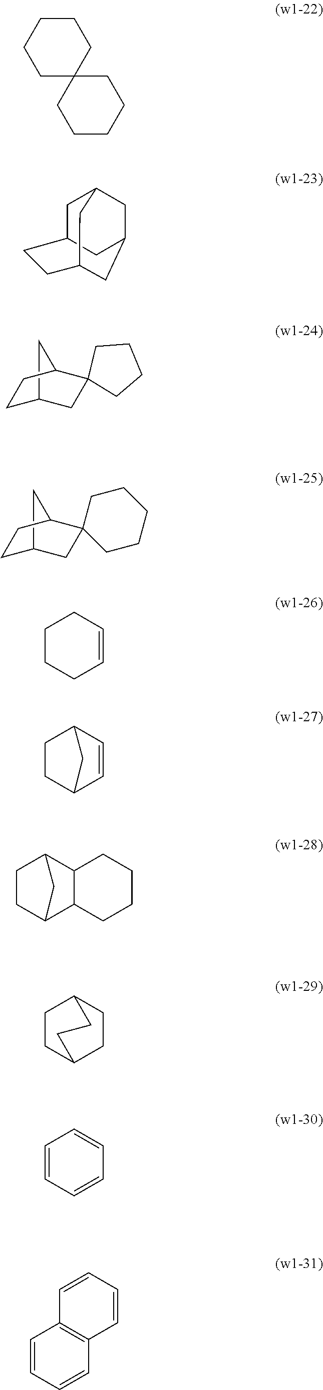

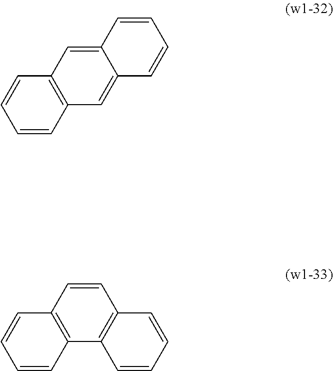

[In the formula (w1-1) to formula (w1-33),
the hydrogen atom included in the group may be substituted with a substituent, and —CH₂— included in the group may be substituted with —O—, —S—, —CO—, —SO—, —NR⁷—, or —SO₂—. the binding site may be any position.]

Examples of the substituent of the cyclic hydrocarbon group included in W¹ include a halogen atom, a cyano group, a nitro group, a haloalkyl group having 1 to 6 carbon atoms, and an alkyl group having 1 to 12 carbon atoms (—CH₂— included in the alkyl group may be substituted with —O—, —CO—, —S—, —SO—, —NR⁷—, or —SO₂—).

Examples of the halogen atom include a fluorine atom, a chlorine atom, a bromine atom, and an iodine atom.

Examples of the haloalkyl group having 1 to 6 carbon atoms include an alkyl fluoride group having 1 to 6 carbon atoms, an alkyl chloride group having 1 to 6 carbon atoms, an alkyl bromide group having 1 to 6 carbon atoms, and an alkyl iodide group having 1 to 6 carbon atoms. Examples of the haloalkyl group include a perfluoroalkyl group having 1 to 6 carbon atoms (a trifluoromethyl group, a pentafluoroethyl group, a heptafluoropropyl group, a nonafluorobutyl group, and the like), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, an iodomethyl group, a fluoromethyl group, and a difluoromethyl group. The number of carbon atoms of the haloalkyl group is preferably 1 to 4, and more preferably 1 to 3.

Examples of the alkyl group having 1 to 12 carbon atoms include alkyl groups such as a methyl group, an ethyl group, a propyl group, an isopropyl group, a butyl group, an isobutyl group, a tert-butyl group, a pentyl group, a hexyl group, an octyl group and a nonyl group. The number of carbon atoms of the alkyl group is preferably 1 to 9, more preferably 1 to 6, and still more preferably 1 to 4.

When —CH₂— included in the above-described alkyl group is substituted with —O—, —CO—, —S—, —SO—, —NR⁷—, or —SO₂—, the number of carbon atoms before the substitution is defined as the total number of carbon atoms of the alkyl group.

Examples of the substituted group include a hydroxy group, a carboxy group, an oxy group, a carbonyl group, an alkoxy group, an alkoxycarbonyl group, an alkylcarbonyl group, an alkylcarbonyloxy group, a thiol group, an alkylthio group, an alkylsulfonyl group, an alkoxycarbonyloxy group, an alkoxyalkoxy group, an amino group, an alkylamino group, an alkylpeptide group, and a group obtained by combining two or more of these groups. Examples of these substituted groups include those that are the same as described in the present specification as long as the upper limit of the number of carbon atoms permits.

Examples of the alkoxy group include an alkoxy group having 1 to 11 carbon atoms. Examples of the alkoxycarbonyl group include an alkoxycarbonyl group having 2 to 11 carbon atoms. Examples of the alkylcarbonyl group include an alkylcarbonyl group having 2 to 12 carbon atoms. Examples of the alkylcarbonyloxy group include an alkylcarbonyloxy group having 2 to 11 carbon atoms. Examples of the alkylthio group include an alkylthio group having 1 to 11 carbon atoms. Examples of the alkylsulfonyl group include an alkylsulfonyl group having 1 to 11 carbon atoms. Examples of the alkoxycarbonyloxy group include an alkoxycarbonyloxy group having 1 to 10 carbon atoms. Examples of the alkoxyalkoxy group include an alkoxyalkoxy group having 1 to 10 carbon atoms. Examples of the alkylamino group include an alkylamino group having 1 to 11 carbon atoms. Examples of the alkyl peptide group include an alkyl peptide group having 1 to 11 carbon atoms. Examples of these substituted groups include those that are the same as described in the present specification as long as the upper limit of the number of carbon atoms permits.

The substituent that may be possessed by W¹ is preferably a halogen atom, a haloalkyl group having 1 to 3 carbon atoms, or an alkyl group having 1 to 6 carbon atoms (—CH₂— in the alkyl group may be substituted with —O— or —CO—); more preferably a fluorine atom, an iodine atom, a fluorinated alkyl group having 1 to 3 carbon atoms, an alkyl group having 1 to 3 carbon atoms, a hydroxy group, an alkoxy group having 1 to 3 carbon atoms, or a carboxy group; still more preferably a fluorine atom, an iodine atom, a trifluoromethyl group, a methyl group, an ethyl group, a hydroxy group, a methoxy group, an ethoxy group, or a carboxy group; and yet more preferably a hydroxy group, a fluorine atom, or an iodine atom.

The number of the substituent that may be possessed by W¹ is not particularly limited, but is preferably 1, 2, or 4 when W¹ has a substituent.

W¹ is preferably a single bond or a cyclic hydrocarbon group having 3 to 18 carbon atoms (a hydrogen atom in the cyclic hydrocarbon group may be substituted with a substituent, and —CH₂— in the cyclic hydrocarbon group may be substituted with —O—, —S—, —CO—, —SO—, —NR⁷—, or —SO₂—); more preferably a single bond or a cyclic hydrocarbon group represented by a formula (W-0) (hereinafter may be referred to as “ring W-0”); and still more preferably a single bond or a cyclic hydrocarbon group represented by a formula (W-1) (hereinafter may be referred to as “ring W-1”).

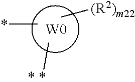

[In the formula (W-0),
W0 represents a cyclic hydrocarbon group having 3 to 10 carbon atoms, the carbon atom included in the cyclic hydrocarbon group may form a double bond between two adjacent carbon atoms, —CH₂— included in the cyclic hydrocarbon group may be substituted with —O—, —S—, —CO—, —SO—, —NR⁷— or —SO₂—, and the hydrogen atom included in the cyclic hydrocarbon group may be substituted with a substituent.

R²² represents a halogen atom, a haloalkyl group having 1 to 6 carbon atoms, or an alkyl group having 1 to 12 carbon atoms, —CH₂— included in the alkyl group may be substituted with —O—, —CO—, —S—, —SO—, —NR⁷—, or —SO₂—, the alkyl group may be bonded to a carbon atom included in W¹ to form an alkanediyl bridge, —CH₂— included in the alkanediyl bridge may be substituted with —O—, —CO—, —S—, —SO—, —NR⁷—, or —SO₂—, the carbon atom included in the alkanediyl bridge may form a double bond between two adjacent carbon atoms, and the alkanediyl bridge may have a substituent.

m22 represents an integer of 0 to 5, and when m22 is 2 or more, a plurality of R²² may be the same as or different from each other.

* represents a binding site with L¹, and ** represents a binding site with A¹, and there may be two or more binding sites.]

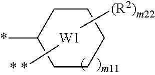

[In the formula (W-1),
W¹ represents a cyclic hydrocarbon group having (5+m11) carbon atoms, the carbon atom included in the cyclic hydrocarbon group may form a double bond between two adjacent carbon atoms, —CH₂— included in the cyclic hydrocarbon group may be substituted with —O—, —S—, —CO—, —SO—, —NR⁷— or —SO₂—, and the hydrogen atom included in the cyclic hydrocarbon group may be substituted with a substituent.

R²² represents a halogen atom, a haloalkyl group having 1 to 6 carbon atoms, or an alkyl group having 1 to 12 carbon atoms, —CH₂— included in the alkyl group may be substituted with —O—, —CO—, —S—, —SO—, —NR⁷—, or —SO₂—, the alkyl group may be bonded to a carbon atom included in W¹ to form an alkanediyl bridge, —CH₂— included in the alkanediyl bridge may be substituted with —O—, —CO—, —S—, —SO—, —NR⁷—, or —SO₂—, the carbon atom included in the alkanediyl bridge may form a double bond between two adjacent carbon atoms, and the alkanediyl bridge may have a substituent.

m11 represents an integer of 0 to 3.

m22 represents an integer of 0 to 5, and when m22 is 2 or more, a plurality of R²² may be the same as or different from each other.

* represents a binding site with L¹, and ** represents a binding site with A1.]

In the ring W-0 or ring W-1, the carbon atom included in the ring W-0 or ring W-1 may form a double bond between two adjacent carbon atoms. The number of double bonds herein may be one, two, three or more, or may constitute an aromatic ring.

Examples of the cyclic hydrocarbon group having 3 to 10 carbon atoms of W0 or the cyclic hydrocarbon group having (5+m11) carbon atoms of W¹ (ring of (5+m11) membered ring) include the same groups as those described above as long as the number of carbon atoms of each group is allowable.

The group in which the alkyl group of R²² is bonded to a carbon atom included in W¹ to form an alkanediyl bridge is a linear or branched alkanediyl group, and examples thereof include a methylene group, an ethylene group, a propanediyl group, a butanediyl group, a pentanediyl group, a hexanediyl group, and a 2-methylene-propanediyl group.

The alkanediyl group is bonded to two carbon atoms included in W¹, and in this case, the alkanediyl group may be bonded to a carbon atom constituting a cyclic hydrocarbon group of W0 in the ring W-0 or W¹ in the ring W-1 to form an alkanediyl bridge, or two or more alkanediyl groups may be present, and a carbon atom constituting one alkanediyl group may be bonded to a carbon atom constituting another alkanediyl group to form an alkanediyl bridge. The substituent described above may be bonded to an alkanediyl bridge. When —CH₂— included in the alkanediyl group is substituted with —O—, —CO—, —S—, —SO—, —NR⁷—, or —SO₂—, —O— or —S— is preferable.

m11 is preferably an integer of 0 to 2, more preferably 0 or 1, and still more preferably 1.

m22 is preferably an integer of 0 to 4, more preferably 0, 1, 2, or 4, and still more preferably 0 or 4.

The cyclic hydrocarbon group represented by the formula (W-0) or (W-1) is preferably a cyclic hydrocarbon group as described below. These cyclic hydrocarbon groups may have the substituent of R²² described above, and preferably have 0 to 4 such substituents. The binding site can be any position.

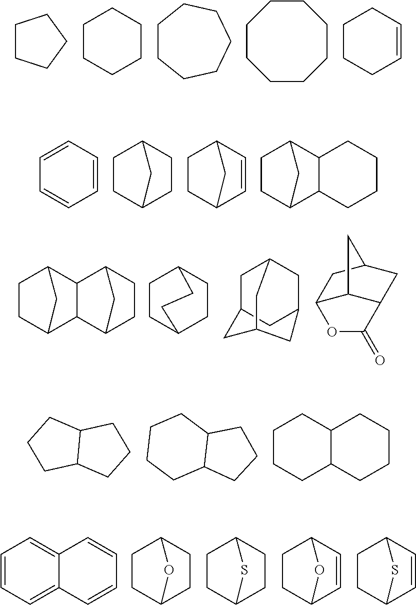

Among these, a cyclic hydrocarbon group represented by the following is preferable.

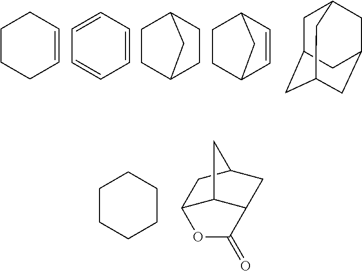

The hydrocarbon group of A¹ may be either a saturated or unsaturated group; examples thereof include linear or a branched chain hydrocarbon group (for example, an alkanediyl group), a monocyclic or polycyclic (a spiro ring, a fused ring, or a bridged ring is included) alicyclic hydrocarbon group, and an aromatic hydrocarbon group; and the hydrocarbon group of A¹ may be the group combining two or more of these groups (for example, a hydrocarbon group formed from an alicyclic hydrocarbon group or an aromatic hydrocarbon group and a chain hydrocarbon group). The number of carbon atoms in the hydrocarbon group is preferably 1 to 19, more preferably 1 to 18, still more preferably 1 to 16, yet more preferably 1 to 14, and further preferably 1 to 12. Examples of these groups include the same groups as those exemplified for L¹ and L².

The number of carbon atoms of the chain hydrocarbon group is preferably 1 to 18, more preferably 1 to 12, still more preferably 1 to 9, yet more preferably 1 to 6, further preferably 1 to 4, and further more preferably 1 to 3.

The number of carbon atoms of the alicyclic hydrocarbon group is preferably 3 to 18, more preferably 3 to 16, still more preferably 3 to 12, and yet more preferably 3 to 10.

The number of carbon atoms of the aromatic hydrocarbon group is preferably 6 to 18, more preferably 6 to 14, and still more preferably 6 to 10.

In A¹, when —CH₂— included in a hydrocarbon group is substituted with —O—, —CO—, —S—, —SO—, —NR⁷—, or —SO₂—, the number of carbon atoms before substitution is defined as the total number of carbon atoms of the hydrocarbon group. In addition, the number may be one or two or more, but is preferably one to three.

Examples of the group in which —CH₂— included in the hydrocarbon group is substituted with —O—, —CO—, —S—, —SO—, —NR⁷—, or —SO₂— include the same groups as those exemplified as the group in which —CH₂— included in the hydrocarbon group of L¹ is substituted with —O—, —CO—, —S—, —SO—, —NR⁷—, or —SO₂—.

Examples of the substituent that may be possessed by the hydrocarbon group of A¹ include the same groups as those exemplified as the substituent that may be possessed by the hydrocarbon group of L¹.

Examples of A¹ include a single bond, a chain hydrocarbon group having 1 to 18 carbon atoms, a cyclic hydrocarbon group having 3 to 18 carbon atoms, or a combination thereof (provided that the chain hydrocarbon group and the cyclic hydrocarbon group may have a substituent, and —CH₂— included in the chain hydrocarbon group and the cyclic hydrocarbon group may be substituted with —O—, —S—, —SO₂—, —SO—, —NR⁷—, or —CO—); preferably a single bond, a chain hydrocarbon group having 1 to 10 carbon atoms, a cyclic hydrocarbon group having 3 to 18 carbon atoms, or a combination thereof (provided that the chain hydrocarbon group and the cyclic hydrocarbon group may have a substituent, and —CH₂— included in the chain hydrocarbon group and the cyclic hydrocarbon group may be substituted with —O—, —S—, —SO₂—, —SO—, —NR⁷—, or —CO—); and more preferably a single bond, a chain hydrocarbon group having 1 to 8 carbon atoms (provided that —CH₂— in the chain hydrocarbon group may be substituted with —O— or —CO—), a group formed by combining a chain hydrocarbon group having 1 to 6 carbon atoms with an alicyclic hydrocarbon group having 3 to 18 carbon atoms (provided that —CH₂— in the chain hydrocarbon group may be substituted with —O— or —CO—, and —CH₂— in the alicyclic hydrocarbon group may be substituted with —O—, —S—, —SO—, —SO₂—, —NR⁷—, or —CO—), or
a group formed by combining a chain hydrocarbon group having 1 to 6 carbon atoms with an aromatic hydrocarbon group having 6 to 18 carbon atoms and optionally having a substituent (provided that —CH₂— in the chain hydrocarbon group may be substituted with —O— or —CO—).

In addition, A¹ is preferably a single bond or a hydrocarbon group having 1 to 20 carbon atoms (the hydrocarbon group may have a substituent, and —CH₂— included in the hydrocarbon group may be substituted with —O—, —CO—, —S—, —SO—, —NR⁷—, or —SO₂—. Provided that at least one of —CH₂— included in the hydrocarbon group is substituted with —O—, —CO—, —S—, —SO—, —NR⁷—, or —SO₂—).

In this case, for example, A¹ is preferably a single bond or *-X^{1A}-A^{1A}-(X^{1A} represents *—CO—O—, *—O—CO—, *—O—CO—O—, *—O—, *—CO—NR⁷—, *—NR⁷—CO—, *—NR⁷—CO—O—, *—O—CO—NR⁷—, or *—NR⁷—. * represents a binding site to W¹. A^{1A} represents a single bond, a chain hydrocarbon group having 1 to 4 carbon atoms, a cyclic hydrocarbon group having 3 to 12 carbon atoms, or a group obtained by combining these groups, and —CH₂— included in the chain hydrocarbon group and the cyclic hydrocarbon group may be substituted with —O—, —S—, —SO₂—, —SO—, —NR⁷—, or —CO—).

In addition, A¹ is more preferably a single bond or ***-L⁰¹¹-X⁰¹-L⁰¹². (X⁰¹ represents —O—, —CO—, —S—, —SO—, —NR⁷—, or —SO₂—. L⁰¹¹ and L⁰¹² each independently represent a single bond or a hydrocarbon group having 1 to 18 carbon atoms and optionally having a substituent, and —CH₂— included in the hydrocarbon group may be substituted with —O—, —CO—, —S—, —SO—, —NR⁷—, or —SO₂—. Provided that the total number of carbon atoms in L⁰¹¹ and L⁰¹² is 0 to 18. *** represents the binding site to W¹), and still more preferably a single bond, ***-X⁰¹-L⁰¹-, or ***-L^{O1}-X^{0l}- (X⁰¹ represents —O—, —CO—, —S—, —SO—, —NR⁷—, or —SO₂—. L⁰¹ represents a single bond or a hydrocarbon group having 1 to 18 carbon atoms and optionally having a substituent, and —CH₂— included in the hydrocarbon group may be substituted with —O—, —CO—, S, —SO—, —NR⁷—, or —SO₂—. *** represents the binding site to W¹).

Examples of the hydrocarbon group having 1 to 18 carbon atoms (the hydrocarbon group may have a substituent, and —CH₂— included in the hydrocarbon group may be substituted with —O—, —CO—, —S—, —SO—, —NR⁷—, or —SO₂—) of L⁰¹¹, L⁰¹², and L⁰¹ include the same groups as those exemplified for A¹ within the range of 1 to 18 carbon atoms.

X⁰¹ is preferably —O—, —S—, —NR⁷—, or —CO—, and more preferably —O— or —CO—.

L⁰¹¹, L⁰¹², and L⁰¹ are each independently preferably a single bond or a hydrocarbon group having 1 to 12 carbon atoms (—CH₂— included in the hydrocarbon group may be substituted with —O—, —CO—, —S—, —SO—, —NR⁷—, or —SO₂—); more preferably a single bond or a chain hydrocarbon group having 1 to 9 carbon atoms (—CH₂— included in the chain hydrocarbon group may be substituted with —O—, —CO—, —S—, —SO—, —NR⁷—, or —SO₂—); yet more preferably a single bond or an alkanediyl group having 1 to 6 carbon atoms (—CH₂— included in the alkanediyl group may be substituted with —O— or —CO—); further preferably a single bond or an alkanediyl group having 1 to 5 carbon atoms (—CH₂— in the alkanediyl group may be substituted with —O— or —CO—); further more preferably a single bond or an alkanediyl group having 1 to 4 carbon atoms (—CH₂— in the alkanediyl group may be substituted with —O— or —CO—); and still further more preferably a single bond or an alkanediyl group having 1 to 3 carbon atoms (—CH₂— in the alkanediyl group may be substituted with —O— or —CO—). Among these, a single bond or *1-L^{01A}-L^{01B}-(L^{01A} represents a single bond, —O—, or —CO—. L01^{B} represents a methylene group, an ethane-1,1-diyl group, a propane-1,1-diyl group, a propane-2,2-diyl group, an oxy group, a carbonyl group, a carbonyloxy group, a carbonyloxymethylene group, an ethyleneoxy group, a methylenecarbonyloxymethylene group, or an ethyleneoxycarbonyl group. *1 represents the binding site to W¹ or X⁰¹) is preferable; and a single bond, a methylene group, an oxy group, or a carbonyl group is more preferable.

Examples of the halogen atom as for R⁴ include a fluorine atom, a chlorine atom, a bromine atom and an iodine atom.

Examples of the hydrocarbon group having 1 to 18 carbon atoms as for R⁴ include a chain hydrocarbon groups such as an alkyl group and an alkanediyl group, an alicyclic hydrocarbon groups, an aromatic hydrocarbon groups, and groups formed by combining these groups.

The alkyl group is a linear or branched alkyl group, and examples thereof include a methyl group, an ethyl group, an n-propyl group, an isopropyl group, an n-butyl group, a sec-butyl group, a tert-butyl group, a pentyl group, a hexyl group, a heptyl group, a 2-ethylhexyl group, an octyl group, a nonyl group, a decyl group, an undecyl group and a dodecyl group.

The alkanediyl group is a linear or branched alkanediyl group, and examples thereof include the same groups as those exemplified for L¹ and L².

The number of carbon atoms in the chain hydrocarbon group is preferably 1 to 12, more preferably 1 to 10, still more preferably 1 to 8, yet more preferably 1 to 6, further preferably 1 to 4, and further more preferably 1 to 3.

Examples of the alicyclic hydrocarbon groups include the same groups as those exemplified for L¹ and L², and specific examples of the monocyclic alicyclic hydrocarbon group include monocyclic cycloalkyl groups such as a cyclopropyl group, a cyclobutyl group, a cyclopentyl group, a cyclohexyl group, a cycloheptyl group, a cyclooctyl group, a cyclononyl group, a cyclodecyl group and a cyclododecyl group. Examples of the polycyclic alicyclic hydrocarbon group include polycyclic cycloalkyl groups such as a decahydronaphthyl group, an adamantyl group and a norbornyl group, and spiro rings having a cycloalkyl group, a norbornyl group or an adamantyl group and a cycloalkyl group spiro-bonded to each group, such as a spirocyclohexane-1,2′-cyclopentyl group or a spiroadamantane-2,3′-cyclopentyl group. The number of carbon atoms of the alicyclic hydrocarbon group is preferably 3 to 18, more preferably 3 to 16, and still more preferably 3 to 12.

Examples of the aromatic hydrocarbon group include a phenyl group, a naphthyl group, a biphenyl group, an anthryl group, a phenanthryl group, a binaphthyl group and the like. The number of carbon atoms of the aromatic hydrocarbon group is preferably 6 to 18, more preferably 6 to 14, and still more preferably 6 to 10.

Examples of the group formed by the combination include groups formed by combining an aromatic hydrocarbon group with a chain hydrocarbon group (for examples, the aromatic hydrocarbon group-alkanediyl group-*, the alkyl group-aromatic hydrocarbon group-*, the alkyl group-aromatic hydrocarbon group-alkanediyl group-*), groups formed by combining an alicyclic hydrocarbon group with a chain hydrocarbon group (for example, the alicyclic hydrocarbon group-alkanediyl group-*, the alkyl group-alicyclic hydrocarbon group-*, the alkyl group-alicyclic hydrocarbon group-alkanediyl group-*), and groups formed by combining an aromatic hydrocarbon group with an alicyclic hydrocarbon group (for example, an aromatic hydrocarbon group-alicyclic hydrocarbon group-*, an alicyclic hydrocarbon group-aromatic hydrocarbon group-*). * represents a binding site. —CH₂— included in each of the above groups may be substituted with —O—, —CO—, —S—, —SO—, —NR⁷—, or —SO₂—.

Examples of the aromatic hydrocarbon group-alkanediyl group-* include aralkyl groups such as a benzyl group and a phenethyl group.

Examples of the alkyl group-aromatic hydrocarbon group-* include a tolyl group, a xylyl group, and a cumenyl group.

Examples of the alicyclic hydrocarbon group-alkanediyl group-* include cycloalkylalkyl groups such as a cyclohexylmethyl group, a cyclohexylethyl group, a 1-(adamantan-1-yl)methyl group, and 1-(adamantan-1-yl)-1-methylethyl group.

Examples of the alkyl group-alicyclic hydrocarbon group-* include cycloalkyl groups having an alkyl group, such as a methylcyclohexyl group, a dimethylcyclohexyl group, and a 2-alkyladamantan-2-yl group.

Examples of the aromatic hydrocarbon group-alicyclic hydrocarbon group-* include a phenylcyclohexyl group.

Examples of the alicyclic hydrocarbon group-aromatic hydrocarbon group-* include a cyclohexylphenyl group.

In combination, two or more of alicyclic hydrocarbon groups, aromatic hydrocarbon groups, and chain hydrocarbon groups may be respectively combined. In addition, any group may be bonded to the benzene ring.

When —CH₂— included in the hydrocarbon group represented by R⁴ is substituted with —O—, —CO—, —S—, —SO—, —NR⁷—, or —SO₂—, the number of carbon atoms before substitution is defined as the total number of carbon atoms of the hydrocarbon group. The number may be either 1, or 2 or more.

Examples of the hydrocarbon group in which —CH₂— is substituted with —O—, —S—, —SO₂—, —SO—, —NR⁷—, or —CO— include a hydroxyl group, a thiol group, a carboxy group, an amino group, an oxy group, a carbonyl group, a thio group, a sulfonyl group, a peptide group, an alkoxy group, an alkoxycarbonyl group, an alkylcarbonyl group, an alkylcarbonyloxy group, an alkoxycarbonyloxy group, an alkoxyalkoxy group, an alkylthio group, an alkylsulfonyl group, an alkylamino group, an alkylpeptide group, an alkanediyloxy group, an alkanediyloxycarbonyl group,
an alkanediylcarbonyl group, an alkanediylcarbonyloxy group, an alkanediyloxycarbonyloxy group, an alkanediylthio group, an alkanediylsulfonyl group, an alkanediylamino group, an alkanediylpeptide group, a cycloalkoxy group, a cycloalkylalkoxy group, an aromatic hydrocarbon group-carbonyloxy group, an aromatic hydrocarbon group-carbonyl group, an aromatic hydrocarbon group-oxy group, and a group combining two or more of these groups. Examples of these substituted groups include the same groups as those exemplified in the present specification as long as the number of carbon atoms is acceptable.

In addition, examples of the group in which —CH₂— included in the alicyclic hydrocarbon group, the aromatic hydrocarbon group, or the group combining them is substituted with —O—, —S—, —SO₂—, —SO—, —NR⁷—, or —CO— include the same groups as those exemplified in the present specification as long as the number of carbon atoms is acceptable.

Examples of the substituent that may be possessed by the hydrocarbon group as for R⁴ include a halogen atom, a cyano group, and a nitro group. —CH₂— in the chain hydrocarbon group, such as the alkyl group, of R⁴ is substituted with —O—, —S—, —CO—, —SO—, —NR⁷—, or —SO₂—, whereby R⁴ can substantially have a substituent, such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an amino group, an alkylthio group, an alkylsulfonyl group, an alkylamino group, or an alkylpeptide group. In addition, when R⁴ is a group obtained by combining an alicyclic hydrocarbon group or an aromatic hydrocarbon group with a chain hydrocarbon group such as an alkyl group, the chain hydrocarbon group such as an alkyl group can serve as the substituent of the alicyclic hydrocarbon group or the aromatic hydrocarbon group.

When R⁴ is a hydrocarbon group having a halogen atom, examples of the hydrocarbon group having a halogen atom of R⁴ include a chain hydrocarbon group having a halogen atom such as a haloalkyl group, an alicyclic hydrocarbon group having a halogen atom, and an aromatic hydrocarbon group having a halogen atom.

Examples of the alkyl group having a halogen atom include a haloalkyl group having 1 to 12 carbon atoms, and examples thereof include a fluorinated alkyl group having 1 to 12 carbon atoms, a chloride alkyl group having 1 to 12 carbon atoms, a bromide alkyl group having 1 to 12 carbon atoms, and an iodide alkyl group having 1 to 12 carbon atoms. Examples of the haloalkyl group include a perfluoroalkyl group having 1 to 12 carbon atoms (trifluoromethyl group, pentafluoroethyl group, heptafluoropropyl group, nonafluorobutyl group, etc.), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, an iodomethyl group and the like. The number of carbon atoms of the haloalkyl group is preferably 1 to 9, more preferably 1 to 6, still more preferably 1 to 4, and yet more preferably 1 to 3.

—CH₂— included in the haloalkyl group may be substituted with —O—, —CO—, or the like, and examples of such a group include a haloalkoxy group, a haloalkoxycarbonyl group, a haloalkylcarbonyl group, and a haloalkylcarbonyloxy group. Specific examples thereof include a haloalkoxy group having 1 to 11 carbon atoms, a haloalkoxycarbonyl group having 2 to 11 carbon atoms, a haloalkylcarbonyl group having 2 to 12 carbon atoms, and a haloalkylcarbonyloxy group having 2 to 11 carbon atoms, and examples thereof include groups in which one or more hydrogen atoms of the groups exemplified above are substituted with halogen atoms.

Examples of R⁴ include a halogen atom, an alkyl group having 1 to 12 carbon atoms, or a haloalkyl group having 1 to 12 carbon atoms (—CH₂— in the alkyl group or haloalkyl group may be substituted with —O— or —CO—); preferably a halogen atom, an alkyl group having 1 to 6 carbon atoms, or a haloalkyl group having 1 to 6 carbon atoms (—CH₂— in the alkyl group or haloalkyl group may be substituted with —O— or —CO—); more preferably a halogen atom, an alkyl group having 1 to 4 carbon atoms (—CH₂— in the alkyl group may be substituted with —O— or —CO—), or a haloalkyl group having 1 to 4 carbon atoms; still more preferably an iodine atom, a fluorine atom, a hydroxy group, an alkoxy group having 1 to 3 carbon atoms, a hydroxyalkyl group having 1 to 3 carbon atoms, a carboxy group, or a fluorinated alkyl group having 1 to 3 carbon atoms; yet more preferably iodine atom, a fluorine atom, a hydroxy group, an alkoxy group having 1 to 3 carbon atoms, a carboxy group, or a perfluoroalkyl group having 1 to 3 carbon atoms; and further more preferably an iodine atom, a fluorine atom, a hydroxy group, a methoxy group, an ethoxy group, or a trifluoromethyl group.

The acid stable hydrocarbon group optionally having a substituent of R³ means a group in which a group including a group represented by R³ is not eliminated when being contacted with an acid (for example, trifluoromethanesulfonic acid), specifically, a group in which COOR³ is not changed to COOH.

Examples of the substituent of R³ that may be possessed by the acid stable hydrocarbon group include a halogen atom, a cyano group, a nitro group, and an alkyl group having 1 to 12 carbon atoms and optionally having a halogen atom (—CH₂— included in the alkyl group may be substituted with —O— or —CO—).

Examples of the halogen atom include a fluorine atom, a chlorine atom, a bromine atom, and an iodine atom.

Examples of the alkyl group optionally having a halogen atom include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a chloromethyl group, a bromomethyl group, and an iodomethyl group. The number of carbon atoms in the alkyl group is preferably 1 to 9, more preferably 1 to 6, still more preferably 1 to 4, and yet more preferably 1 to 3.

Examples of the group in which —CH₂— included in the alkyl group is substituted with —O— or —CO— include the same groups as those exemplified in the present specification as long as the number of carbon atoms is acceptable.

Examples of the acid stable hydrocarbon group optionally having a substituent of R³ include a group represented by a formula (10a-1) or a formula (10a-2).

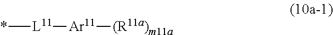

[In the formula (10a-1), all reference numerals are the same as defined above]

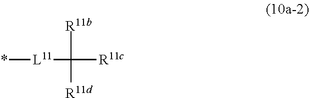

[In the formula (10a-2), all reference numerals are the same as defined above]

Examples of the alkanediyl group in L¹¹ include: linear alkanediyl groups such as a methylene group, an ethylene group, a propane-1,3-diyl group, a butane-1,4-diyl group, a pentane-1,5-diyl group, and a hexane-1,6-diyl group; and
branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group.

L¹¹ is preferably a single bond or an alkanediyl group having 1 to 3 carbon atoms, and more preferably a single bond or a methylene group.

Examples of the aromatic hydrocarbon group of Ar¹¹ include a phenyl group, a naphthyl group, a biphenyl group, a biphenyl group, an anthryl group, a phenanthryl group, a binaphthyl group, and a fluorenyl group. The number of carbon atoms of the aromatic hydrocarbon group is preferably 6 to 18, more preferably 6 to 14, and still more preferably 6 to 10.

m11a is preferably an integer of 0 to 7, more preferably an integer of 0 to 6, still more preferably an integer of 0 to 5, and still more preferably an integer of 0 to 3.

Examples of the halogen atom and the alkyl group optionally having a halogen atom in R^{11a}, R^{11b}, R^{11c}, and R^{11d} include the same groups as those described for the substituent of R³.

R^{11a} is preferably a fluorine atom, an iodine atom, or an alkyl group having 1 to 6 carbon atoms and optionally having a halogen atom (—CH₂— included in the alkyl group may be substituted with —O— or —CO—), more preferably a fluorine atom, an iodine atom, a fluorinated alkyl group having 1 to 4 carbon atoms, a hydroxy group, an alkyl group having 1 to 4 carbon atoms, an alkoxy group having 1 to 4 carbon atoms, a hydroxyalkyl group having 1 to 4 carbon atoms, or a hydroxyalkoxy group having 1 to 4 carbon atoms.

Examples of the hydrocarbon group of R^{11b} and R^{11c} include a chain hydrocarbon group such as an alkyl group, an alicyclic hydrocarbon group, an aromatic hydrocarbon group, and a group formed by combining these groups.

Examples of the alkyl group include alkyl groups such as a methyl group, an ethyl group, a propyl group, an isopropyl group, a butyl group, an isobutyl group, a tert-butyl group, a pentyl group, a hexyl group, an octyl group, and a nonyl group.

The number of carbon atoms of the chain hydrocarbon group is preferably 1 to 12, more preferably 1 to 9, still more preferably 1 to 6, yet more preferably 1 to 4, and further preferably 1 to 3.

Examples of the alicyclic hydrocarbon group include the same groups as those exemplified for L¹ and L². Specific examples thereof include: monocyclic cycloalkyl groups such as a cyclopropyl group, a cyclobutyl group, a cyclopentyl group, a cyclohexyl group, a cyclooctyl group, a cyclononyl group, a cyclodecyl group, and a cyclododecyl group; polycyclic cycloalkyl groups such as a norbornyl group and an adamantyl group; and spirocyclic groups having a cycloalkyl group bonded to cycloalkyl groups such as a spirocyclohexane-1,2′-cyclopentane group and a spiroadamantane-2,3′-cyclopentane group, a norbornyl group, or an adamantyl group via a spiro bond. The number of carbon atoms of the alicyclic hydrocarbon group is preferably 3 to 18, more preferably 3 to 16, and still more preferably 3 to 12.

Examples of the aromatic hydrocarbon group include a phenyl group, a naphthyl group, a biphenyl group, an anthryl group, a phenanthryl group, a binaphthyl group, and a fluorenyl group. The number of carbon atoms of the aromatic hydrocarbon group is preferably 6 to 18, more preferably 6 to 14, and still more preferably 6 to 10.

Examples of the group formed by combining these groups include a group in which an alicyclic hydrocarbon group such as a cyclopentylmethyl group and an adamantylmethyl group is combined with a chain hydrocarbon group, and a group in which an aromatic hydrocarbon group such as a benzyl group and a phenethyl group is combined with a chain hydrocarbon group.

In the case of the group formed by combination, two or more of the above groups may be combined, and a group having a different valence or the like (divalent or trivalent group (chain hydrocarbon group, alicyclic saturated hydrocarbon group, and aromatic hydrocarbon group), and the like) may be included in the above group.

When the alicyclic hydrocarbon group formed by bonding R^{11b} and R^{11c} or R^{11b}, R^{11c}, and R^{11d} to each other has an alkyl group, the number of carbon atoms excluding the number of carbon atoms of the alkyl group is defined as the total number of carbon atoms of the alicyclic hydrocarbon group.

When R^{11b} or R^{11c} is a hydrocarbon group, the number of carbon atoms of the hydrocarbon group is each independently preferably 1 to 16, more preferably 1 to 14, and still more preferably 1 to 12. When R^{11b} and R^{11c} or R^{11b}, R^{11c}, and R^{11d} are bonded to each other to form an alicyclic hydrocarbon group, the number of carbon atoms of the alicyclic hydrocarbon group is each independently preferably 3 to 24, more preferably 3 to 18, and still more preferably 3 to 16.

Examples of a halogen atom that may be possessed or the alkyl group optionally having a halogen atom when R^{11b} and R^{11c} or R^{11b}, R^{11c}, and R^{11d} are bonded to each other to form an alicyclic hydrocarbon group include the same groups as described above.

Examples of the alicyclic hydrocarbon group when R^{11b} and R^{11c}, or R^{11b}, R^{11c}, and R^{11d} are bonded to each other to form an alicyclic hydrocarbon group include the groups represented below. The binding site can be any position.

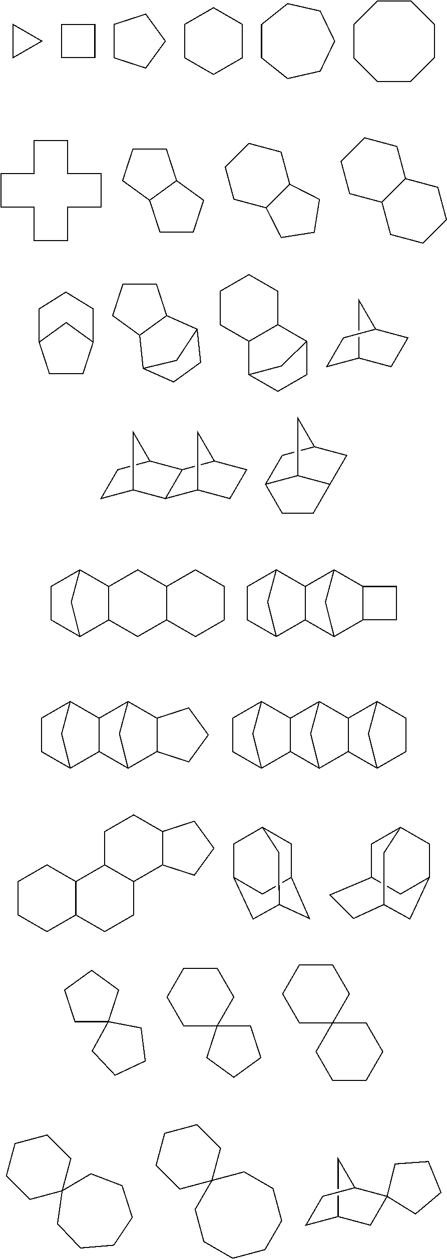

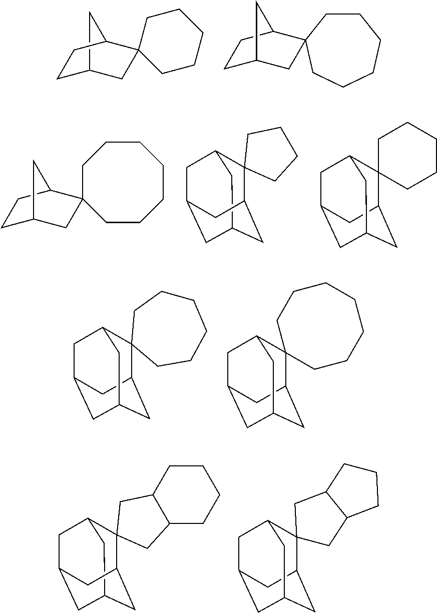

In addition, examples of —C(R^{11b})(R^{11c})(R^{11d}) when R^{11b} and R^{11c} or R^{11b}, R^{11c}, and R^{11d} are bonded to each other to form an alicyclic hydrocarbon group include the following groups. * represents a binding site.

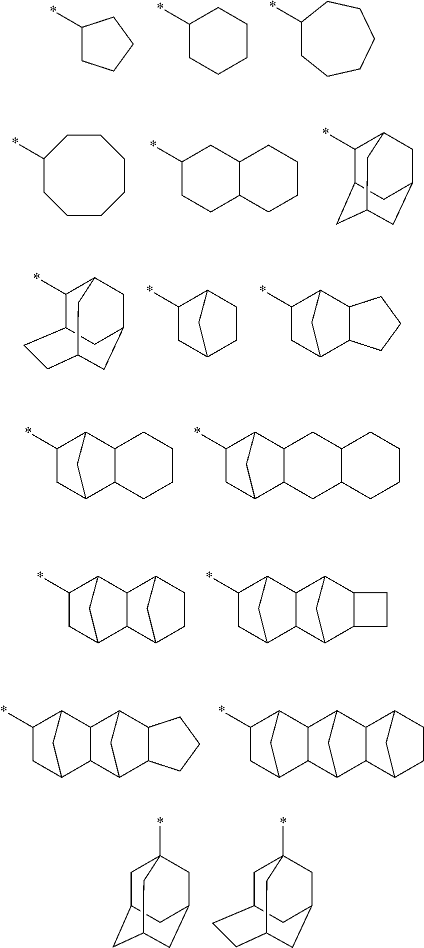

R^{11b} and R^{11c} are each independently a hydrogen atom or a hydrocarbon group having 1 to 14 carbon atoms, or R^{11b} and R^{11c} are bonded to each other to form an alicyclic hydrocarbon group having 3 to 24 carbon atoms and optionally having an alkyl group having 1 to 12 carbon atoms (—CH₂— included in the alkyl group may be substituted with —O— or —CO—), and more preferably each independently a hydrogen atom or a hydrocarbon group having 1 to 12 carbon atoms, or R^{11b} and R^{11c} are bonded to each other to form an alicyclic hydrocarbon group having 3 to 16 carbon atoms and optionally having an alkyl group having 1 to 6 carbon atoms (—CH₂— included in the alkyl group may be substituted with —O— or —CO—).

It is preferable that R^{11d} is a hydrogen atom, or R^{11b}, R^{11c}, and R^{11d} are bonded to each other to form an alicyclic hydrocarbon group having 3 to 24 carbon atoms and optionally having an alkyl group having 1 to 12 carbon atoms (—CH₂— included in the alkyl group may be substituted with —O— or —CO—), and it is more preferable that R^{11d} is a hydrogen atom, or R^{11b}, R^{11c}, and R^{11a} are bonded to each other to form an alicyclic hydrocarbon group having 3 to 16 carbon atoms and optionally having an alkyl group having 1 to 6 carbon atoms (—CH₂— included in the alkyl group may be substituted with —O— or —CO—).

When the alicyclic hydrocarbon group has an alkyl group (—CH₂— included in the alkyl group may be substituted with —O— or —CO—), the bonding position of the alkyl group is bonded to a carbon atom other than a carbon atom bonded to an oxygen atom or a carbonyl group.

Among these, the acid stable hydrocarbon group optionally having a substituent of R³ is preferably a group including a cyclic hydrocarbon group having 3 to 18 carbon atoms and having at least one selected from the group consisting of a halogen atom and a haloalkyl group having 1 to 12 carbon atoms (the cyclic hydrocarbon group may have a substituent other than a halogen atom and a haloalkyl group).

The cyclic hydrocarbon group included in R³ may be any of an alicyclic hydrocarbon group, an aromatic hydrocarbon group, and a group obtained by combining these groups, and examples thereof include the same groups as those described above.

Examples of the halogen atom and the haloalkyl group included in R³ include the same groups as those described above. Among these, a fluorine atom, an iodine atom, or a fluorinated alkyl group having 1 to 3 carbon atoms is preferable, a fluorine atom or an iodine atom is more preferable, and an iodine atom is still more preferable. The number of substitution of halogen atoms and haloalkyl groups is preferably 1 to 4, and more preferably 2 to 3.

The substituent other than the halogen atom and the haloalkyl group that may be possessed by the cyclic hydrocarbon group included in R³ is preferably an alkyl group having 1 to 6 carbon atoms (—CH₂— included in the alkyl group may be substituted with —O— or —CO—), and more preferably an alkyl group having 1 to 3 carbon atoms, a hydroxy group, a methoxy group, an ethoxy group, or a carboxy group.

Specific examples of R³ include the following. * represents a binding site.

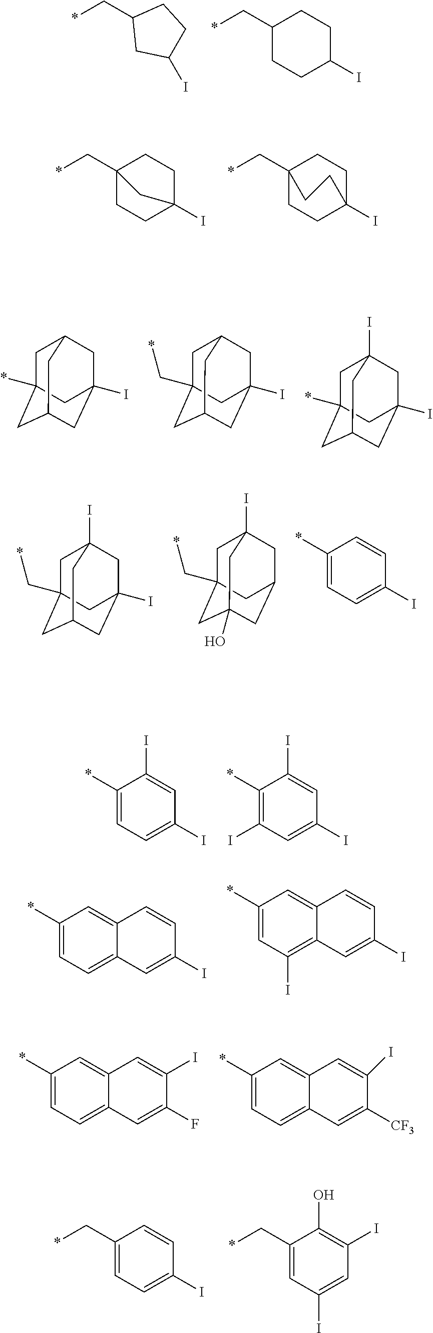

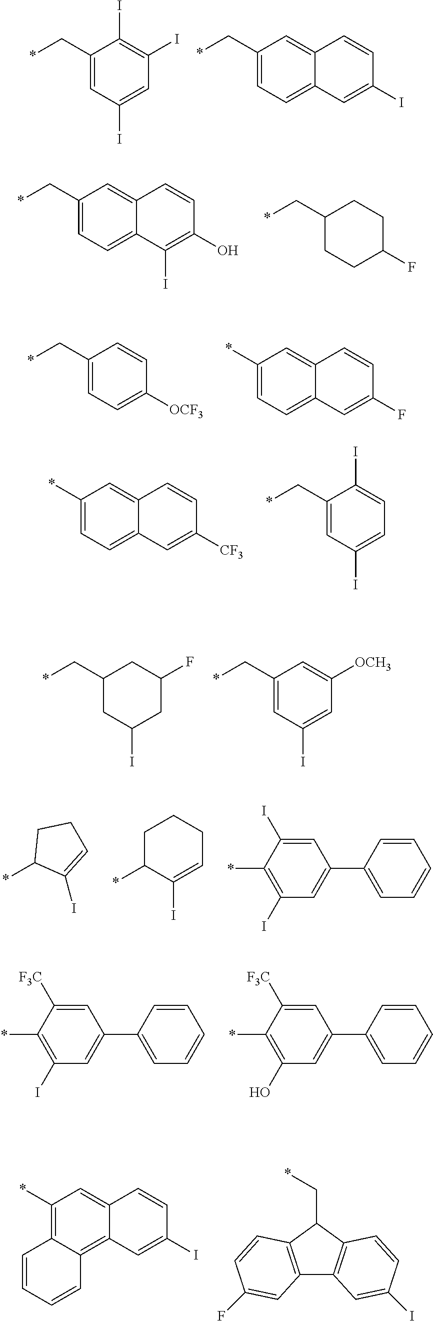

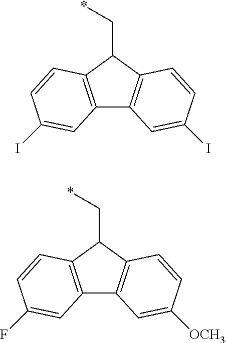 [SMILES: *CC1c2ccc(I)cc2-c2cc(I)ccc21 ; *CC1c2ccc(F)cc2-c2cc(OC)ccc21]

The bonding position of A¹ to the benzene ring may be any of o position, m position, and p position with respect to the bonding position of L². Among these, the bonding position of A¹ to the benzene ring is preferably the p-position or the m-position, and more preferably the p-position with respect to the bonding position of L².

m2 is preferably 1, 2, or 3 and more preferably 1 or 2.

The bonding position of COOR³ to the benzene ring may be any of o position, m position, and p position with respect to the bonding position of A¹. Among these, the bonding position of COOR³ to the benzene ring is preferably at o position or m position with respect to the bonding position of A¹.

The bonding position of R⁴ to the benzene ring may be any of o position, m position, and p position with respect to the bonding position of A¹. Among these, at least one bonding position of R⁴ to the benzene ring is preferably o position or p position with respect to the bonding position of A¹.

X¹ is preferably a single bond, —O—, —CO—, —NR⁷—, a phenylene group optionally having a substituent, or a combination thereof, and more preferably a single bond, *—O—**, *—CO—O—**, *—O—CO—O—**, *—CO—NR⁷—**, *—O—CO—NR⁷—**, or *-Ax-Ph-Ay-**.

In the above group, * and ** represent a binding site, and * represents a binding site with the carbon atom to which R⁰ is bonded. Ax and Ay each independently represent one or more bond species selected from the group consisting of a single bond, an ether bond, a thioether bond, an amide bond, an ester bond, and a carbonic acid ester bond. Ph represents a phenylene group optionally having a substituent.

When X¹ is a group represented by *-Ax-Ph-Ay-**, preferred is a linking group represented by the following formula (X1).

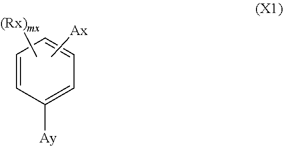

[In the formula (X¹),
Ax and Ay represent the same meaning as described above.

Rx represents a halogen atom, a hydroxy group, an alkyl fluoride group having 1 to 6 carbon atoms, an alkyl group having 1 to 18 carbon atoms, or an alkoxy group having 1 to 6 carbon atoms,
mx represents an integer of 0 to 4, and when mx is an integer of 2 or more, a plurality of Rx may be the same as or different from each other.

When one of Ax and Ay is a single bond, the other is preferably one or more selected from the group consisting of an ether bond, a thioether bond, an ester bond, an amide bond, and a carbonic acid ester bond.

When Ax and Ay are amide bonds, —CO—NR⁷— is preferable.

The binding site of Ay in the phenylene group is preferably the m-position or the p-position, and more preferably the p-position, with respect to the binding site of Ax.

Rx is preferably a fluorine atom, an iodine atom, a trifluoromethyl group, a methyl group or an ethyl group.

mx is preferably 0, 1, or 2.

Examples of X¹ include a single bond and groups represented by the following formula (X¹-1) to formula (X¹-10). * and ** represent a binding site, and * represents a binding site with the carbon atom to which R⁰ is bonded.

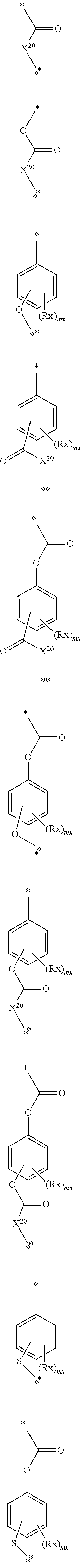

in the formula (X¹-1) to the formula (X¹-10),
* and ** are binding sites, and * represents a binding site to carbon atoms to which R⁰ is bonded,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms, mx represents an integer of 0 to 4,
X²⁰ represents —O— or —NR⁷—, and
R⁷ represents a hydrogen atom or an alkyl group having 1 to 6 carbon atoms.

Specific examples of the groups represented by the formula (X¹-1) to the formula (X¹-10) include the following groups.

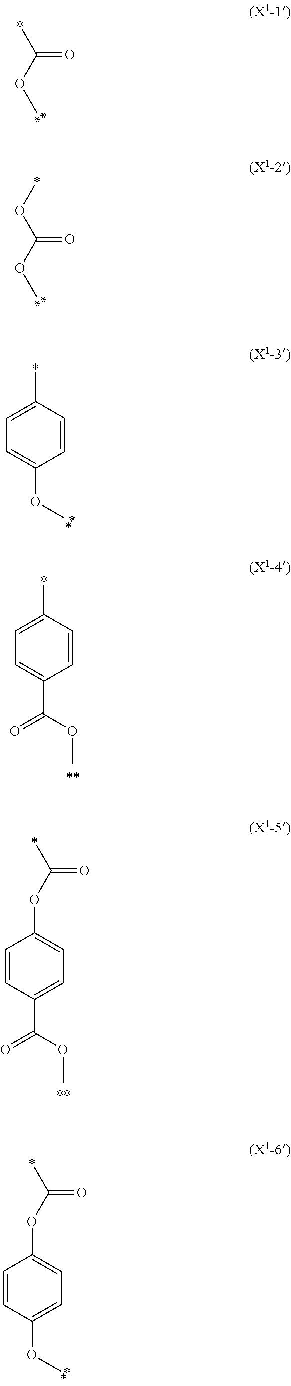

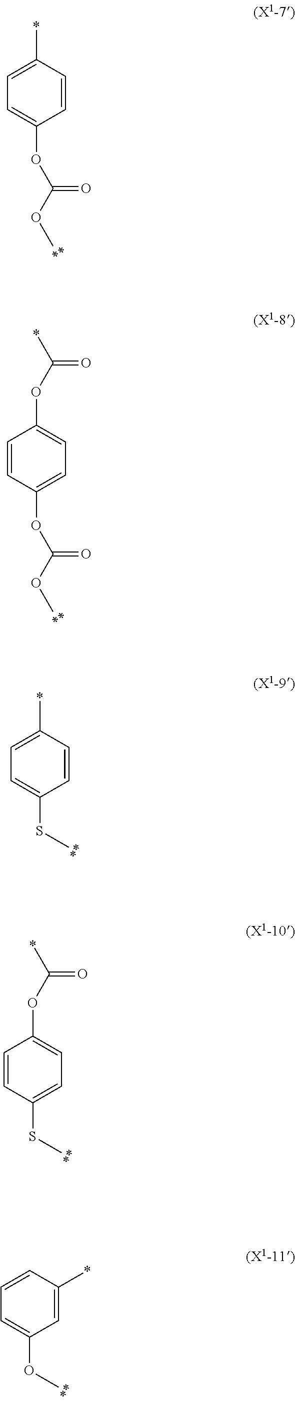

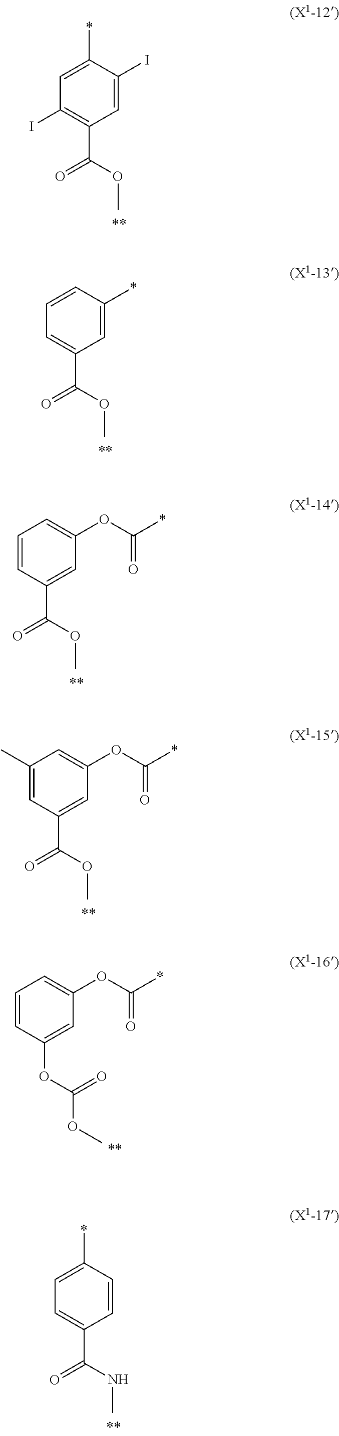 [SMILES: *c1cc(I)c(C(=O)OC)cc1I ; *c1cccc(C(=O)OC)c1 ; *C(=O)Oc1cccc(C(=O)OC)c1 ; *C(=O)Oc1cc(C)cc(C(=O)OC)c1 ; *C(=O)Oc1cccc(OC(=O)OC)c1 ; *c1ccc(C(=O)NC)cc1]

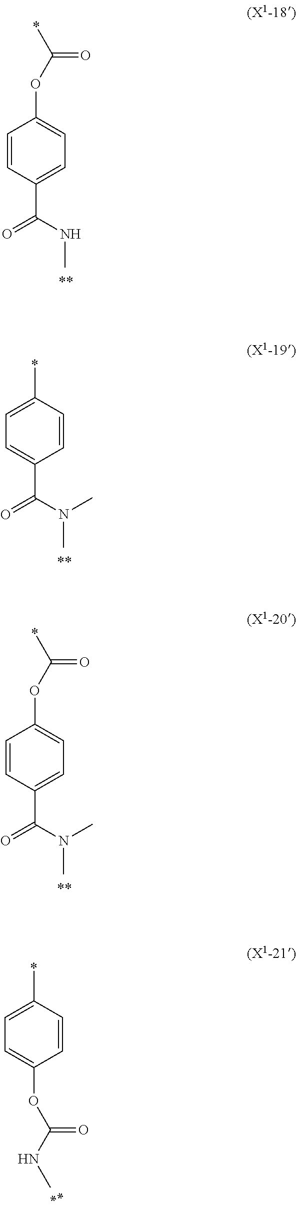 [SMILES: *C(=O)Oc1ccc(C(=O)NC)cc1 ; *c1ccc(C(=O)N(C)C)cc1 ; *C(=O)Oc1ccc(C(=O)N(C)C)cc1 ; *c1ccc(OC(=O)NC)cc1]

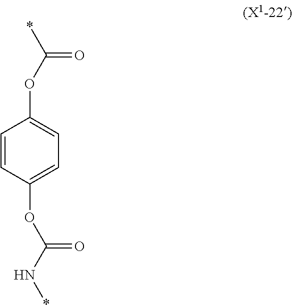 [SMILES: *NC(=O)Oc1ccc(OC(*)=O)cc1]

In particular, X¹ is preferably a single bond or a group represented by any one of a formula (X¹-1′) and a formula (X¹-3′) to a formula (X¹-10′), more preferably a single bond or a group represented by any one ofthe formula (X¹-1), a formula (X¹-4′), a formula (X¹-5′), a formula (X¹-6′), and a formula (X¹-10′), and still more preferably a single bond, a group represented by the formula (X¹-1′), a group represented by the formula (X¹-5′) or a group represented by the formula (X¹-6′).

Examples of the alkyl group of R⁰ include a methyl group, an ethyl group, a n-propyl group, an isopropyl group, a n-butyl group, a sec-butyl group, a tert-butyl group, a n-pentyl group, and a n-hexyl group, and preferably an alkyl group having 1 to 4 carbon atoms, more preferably an alkyl group having 1 to 3 carbon atoms, and still more preferably a methyl group or an ethyl group. The number of carbon atoms of the alkyl group is preferably 1 to 4, more preferably 1 to 3, and still more preferably 1 or 2.

Examples of the halogen atom of R⁰ include a fluorine atom, a chlorine atom, a bromine atom and an iodine atom.

Examples of the alkyl group having a halogen atom of R⁰ include an alkyl fluoride group, an alkyl chloride group, an alkyl bromide group, and an alkyl iodide group, and specific examples thereof include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a perchloromethyl group, a perbromomethyl group, and a periodomethyl group.

R⁰ is preferably a hydrogen atom or an alkyl group having 1 to 4 carbon atoms, more preferably a hydrogen atom or an alkyl group having 1 to 3 carbon atoms, still more preferably a hydrogen atom, a methyl group or an ethyl group, and yet more preferably a hydrogen atom or a methyl group.

Examples of the anion (I) include the following anions.

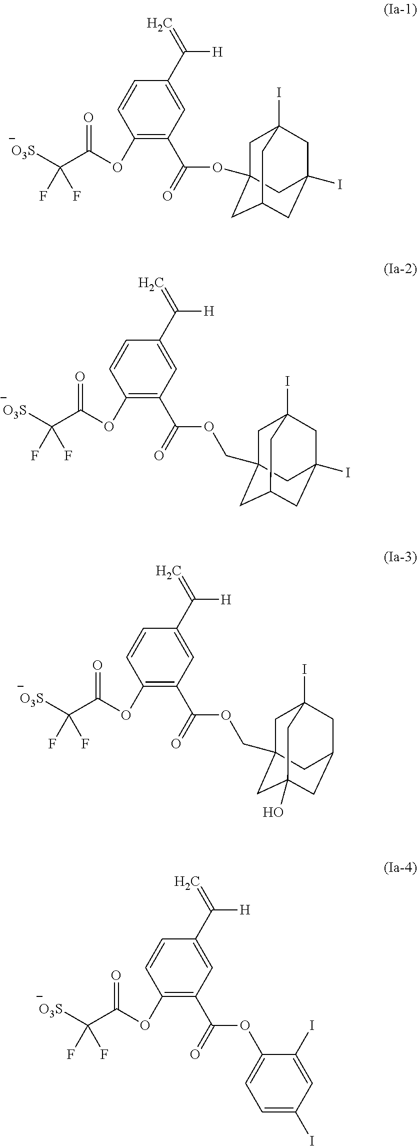

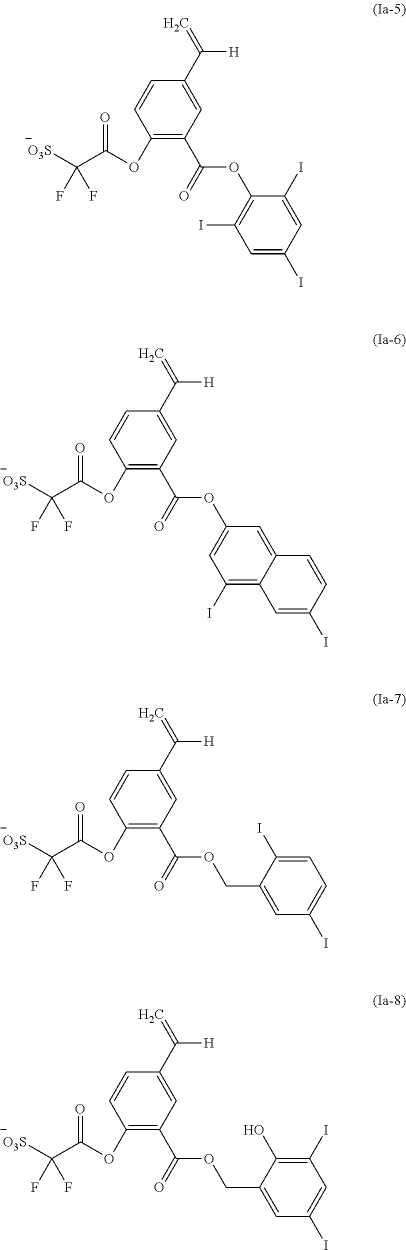

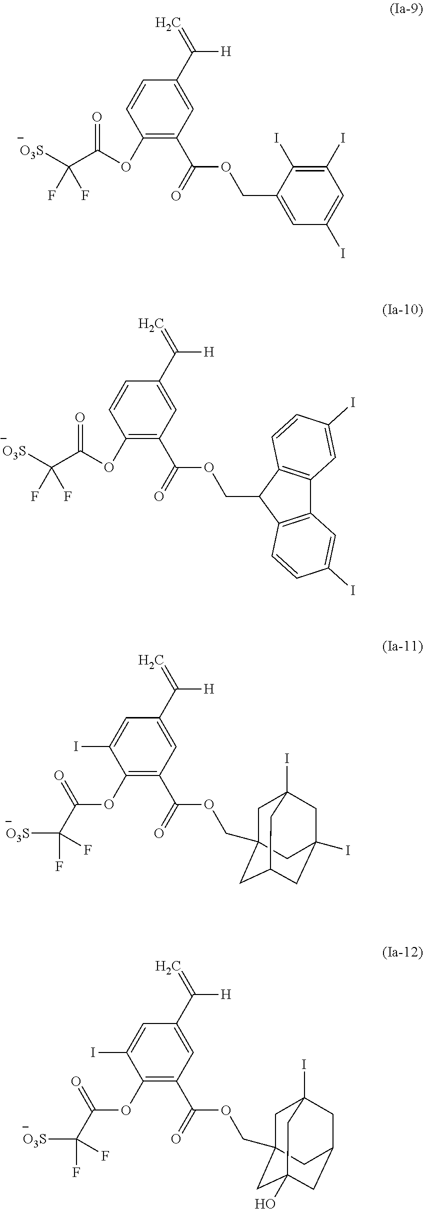

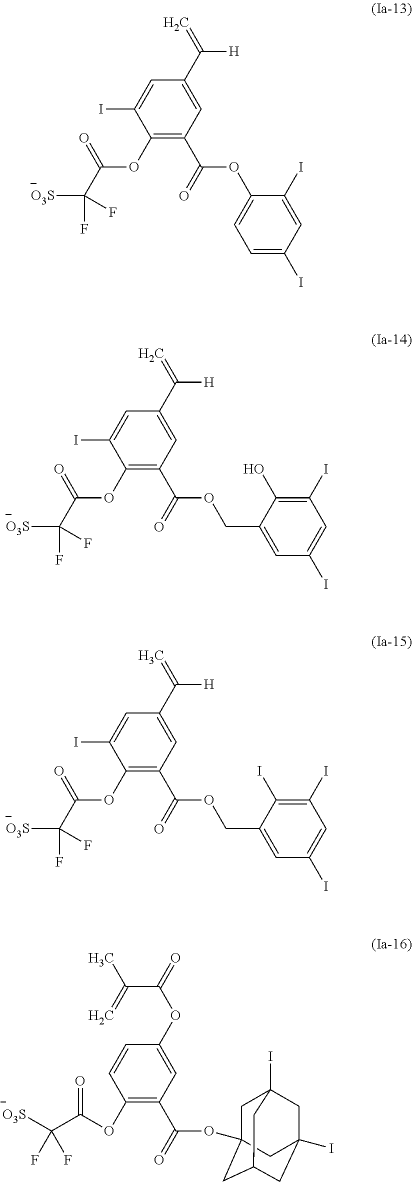

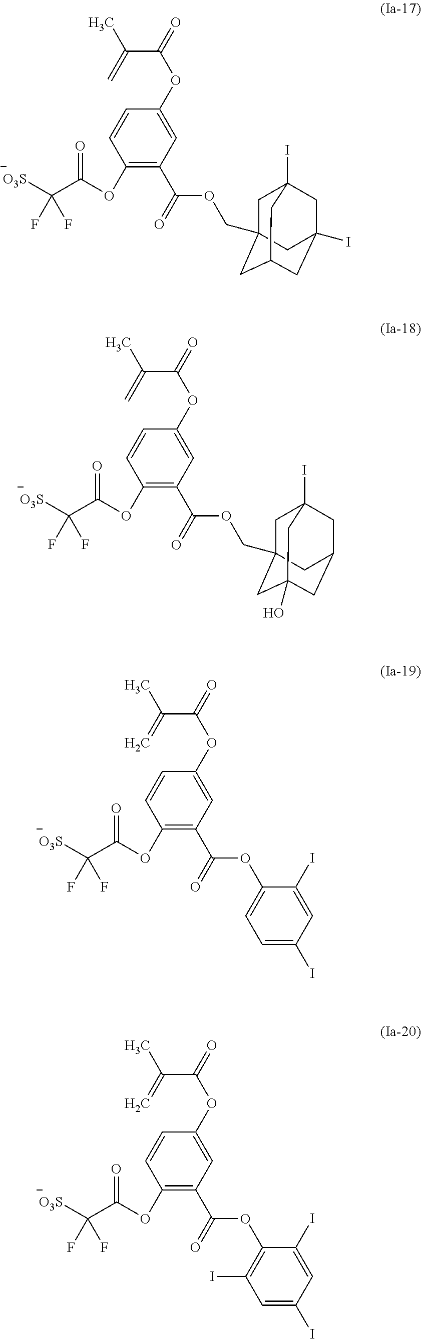

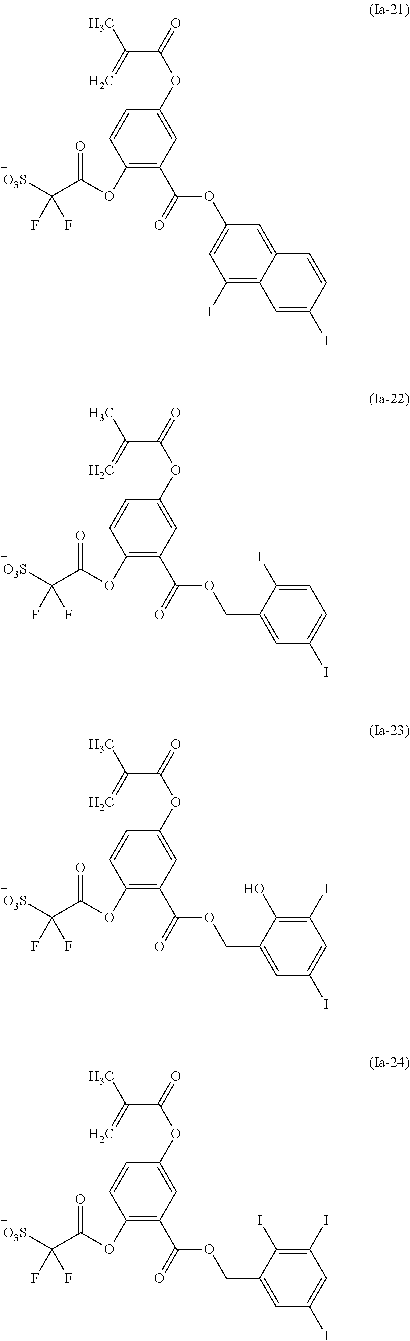

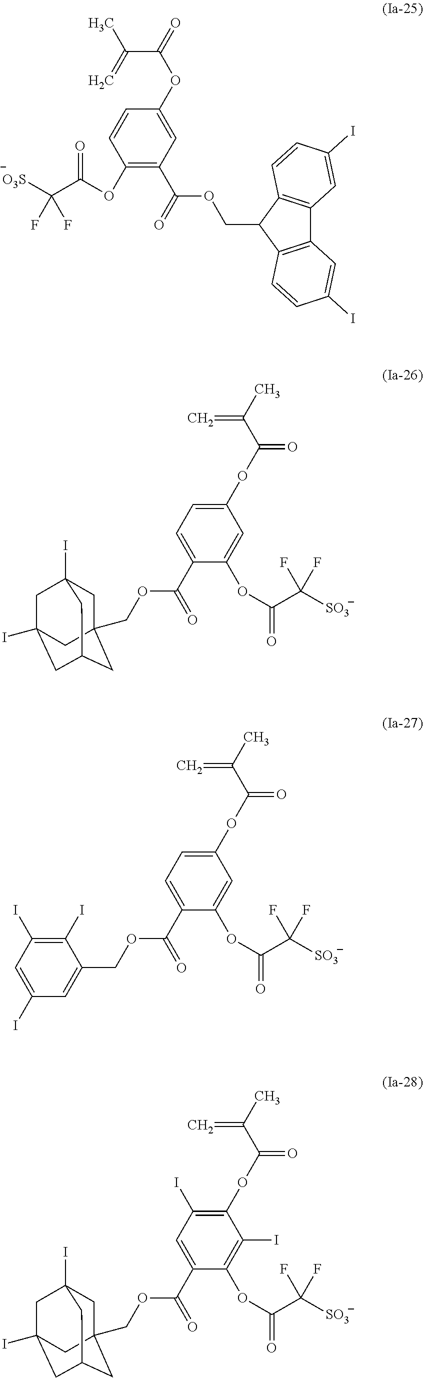

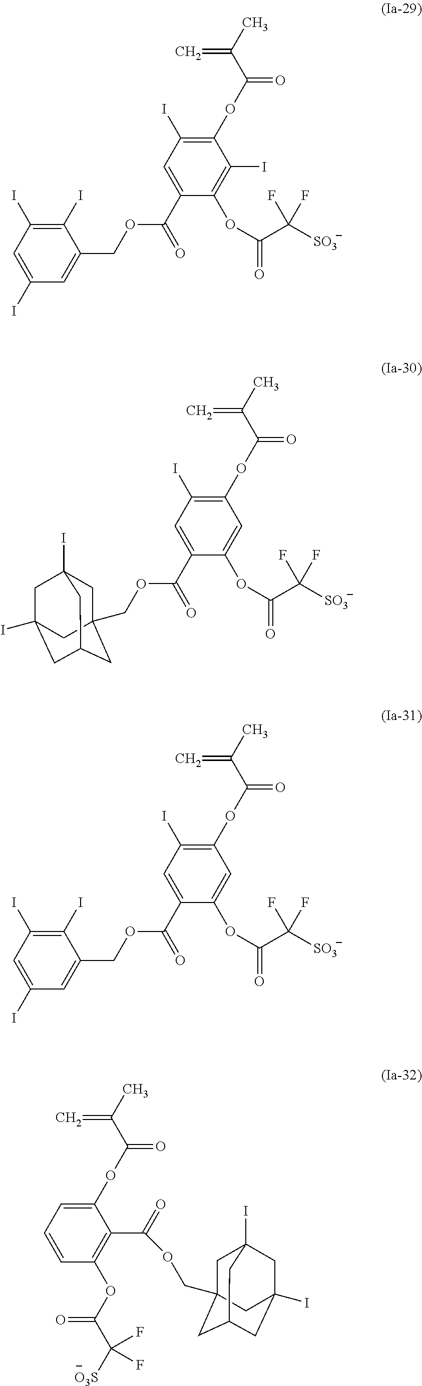

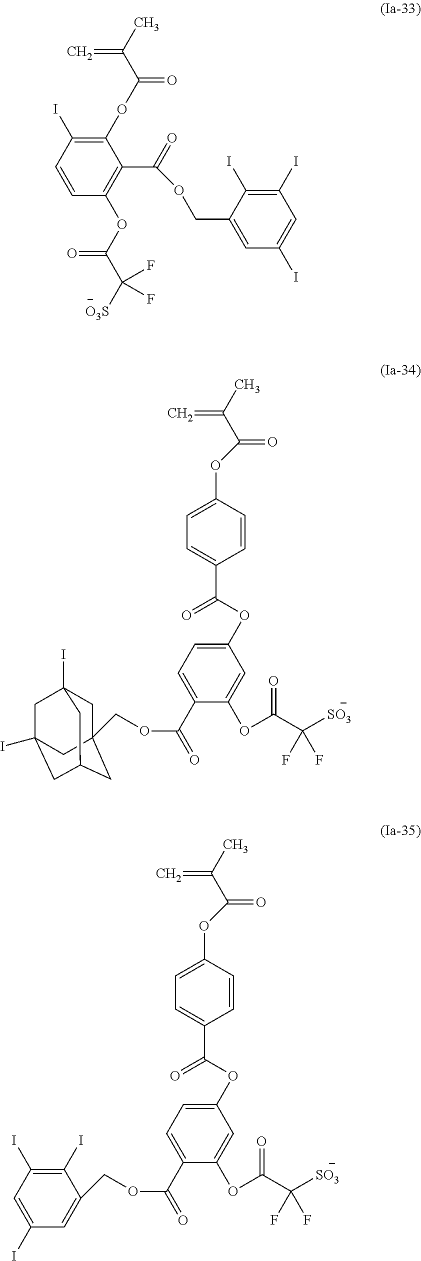

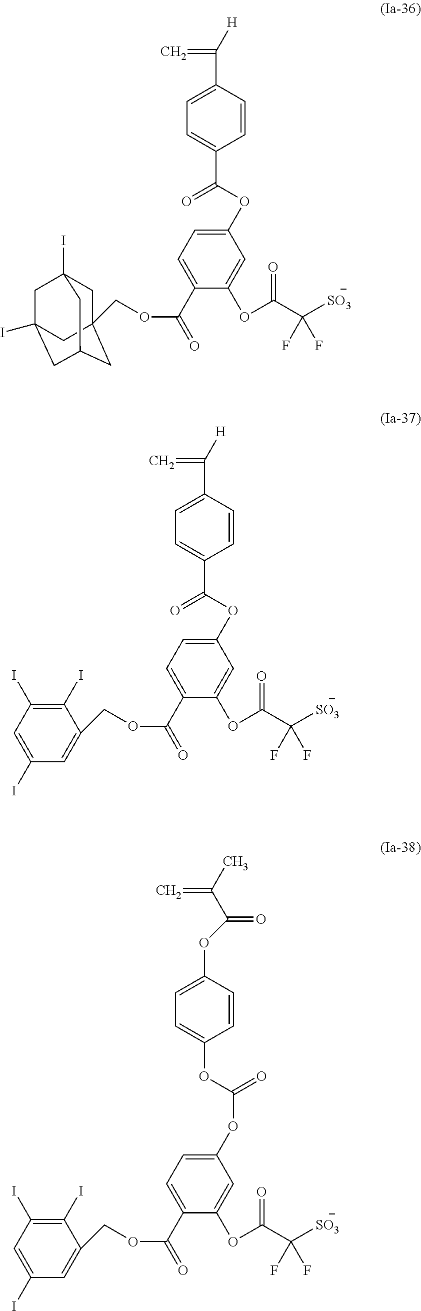

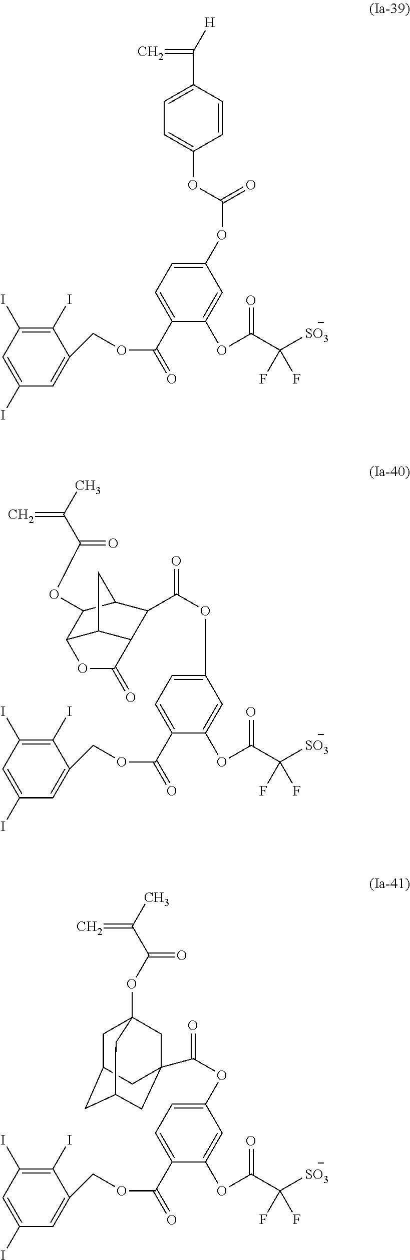

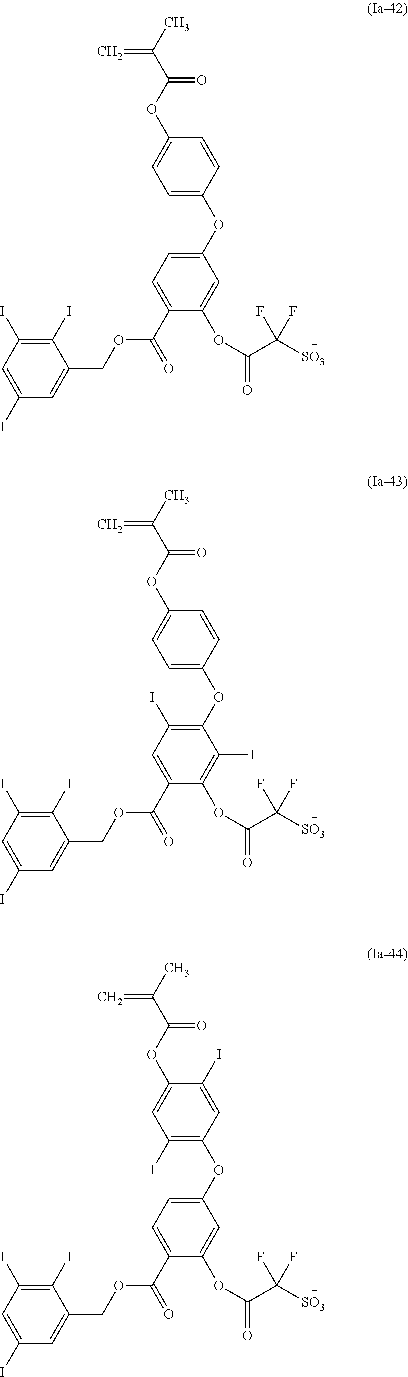

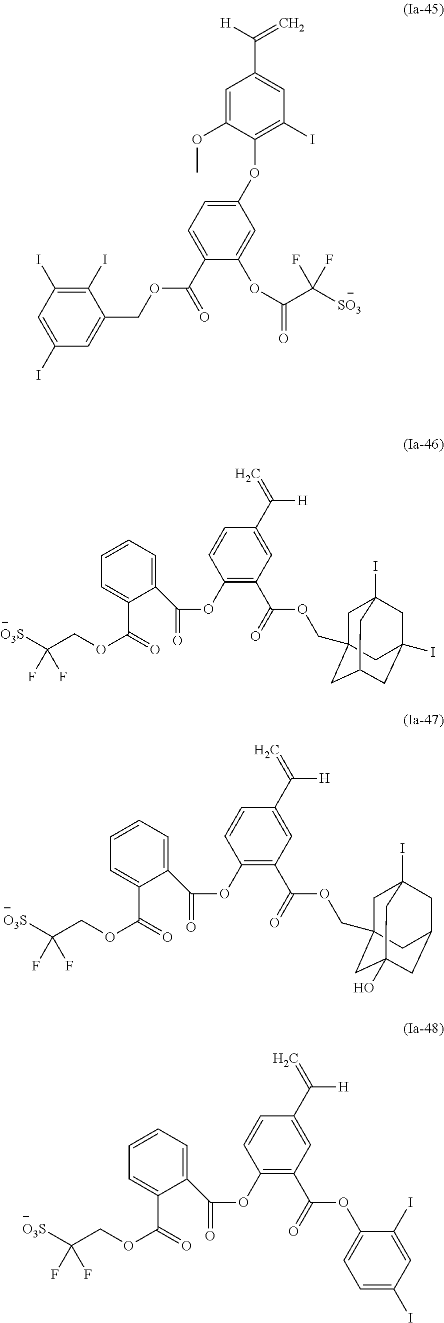

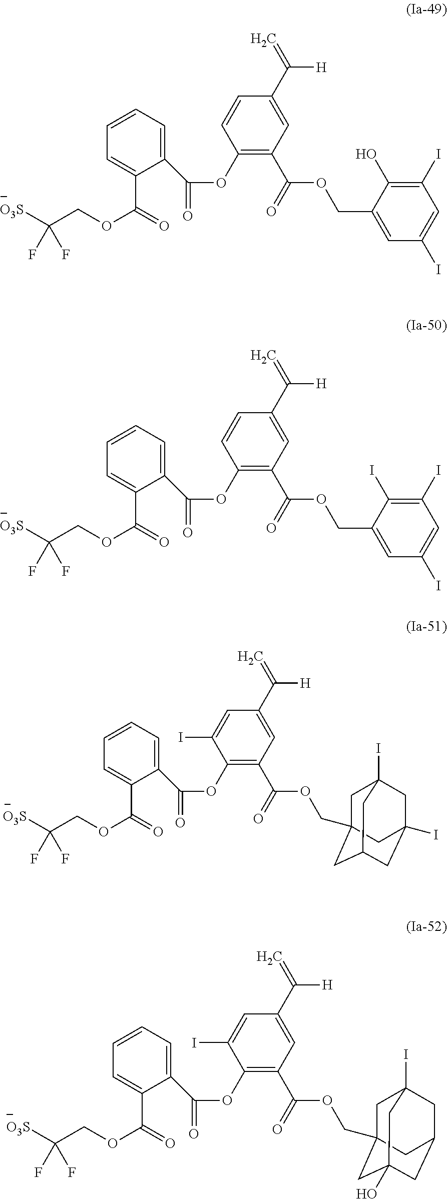

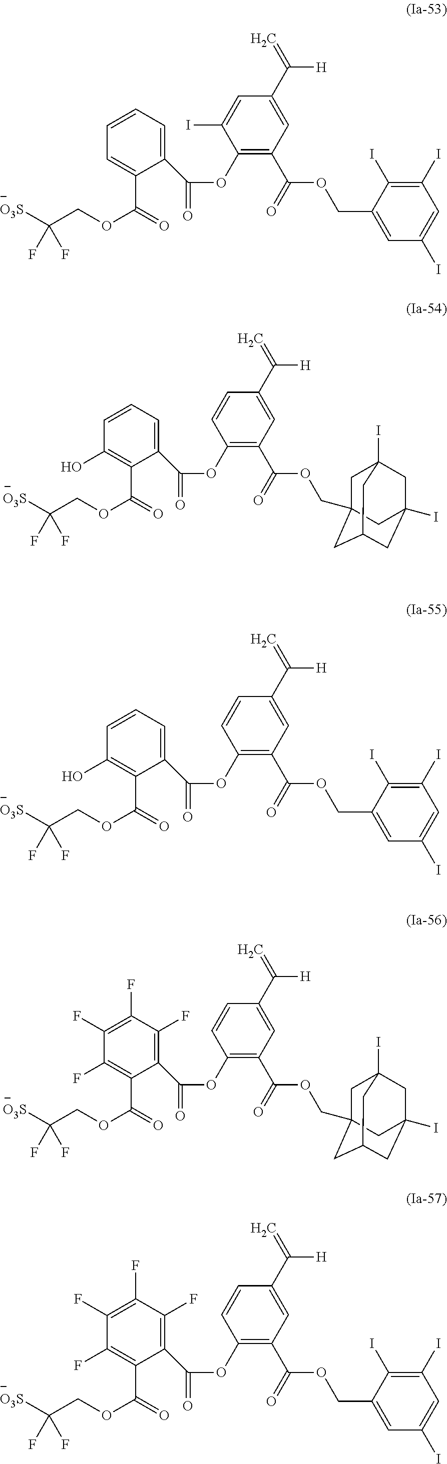

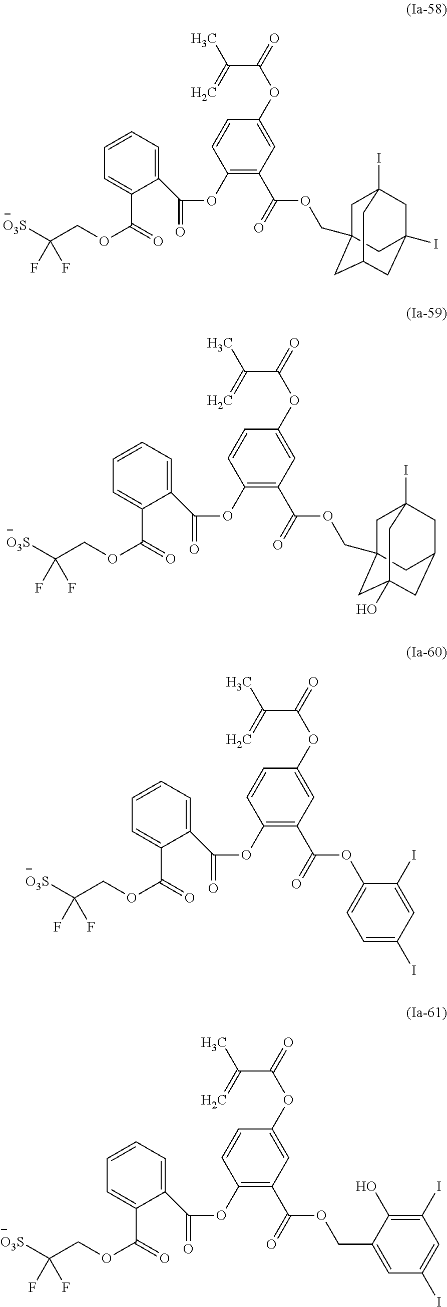

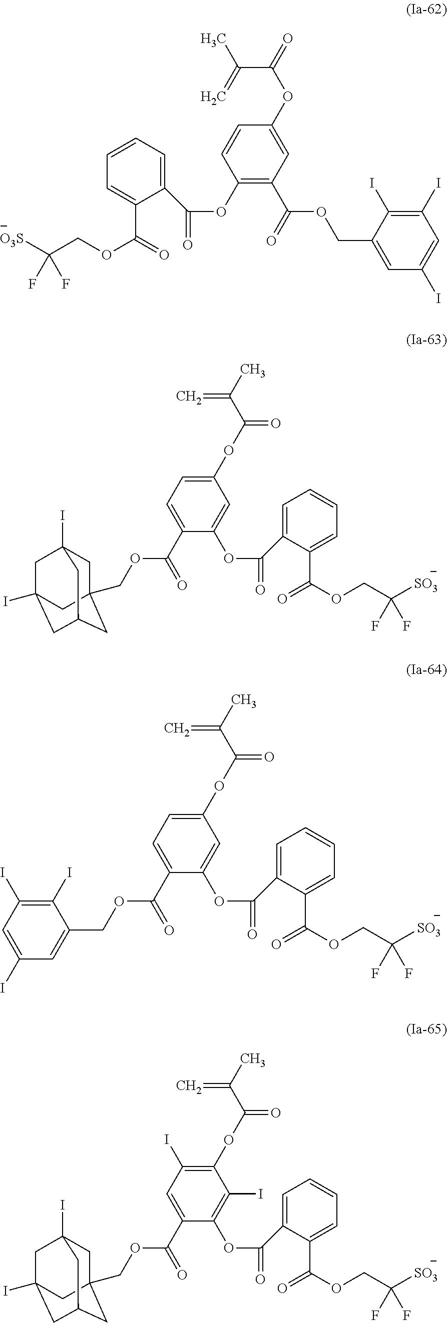

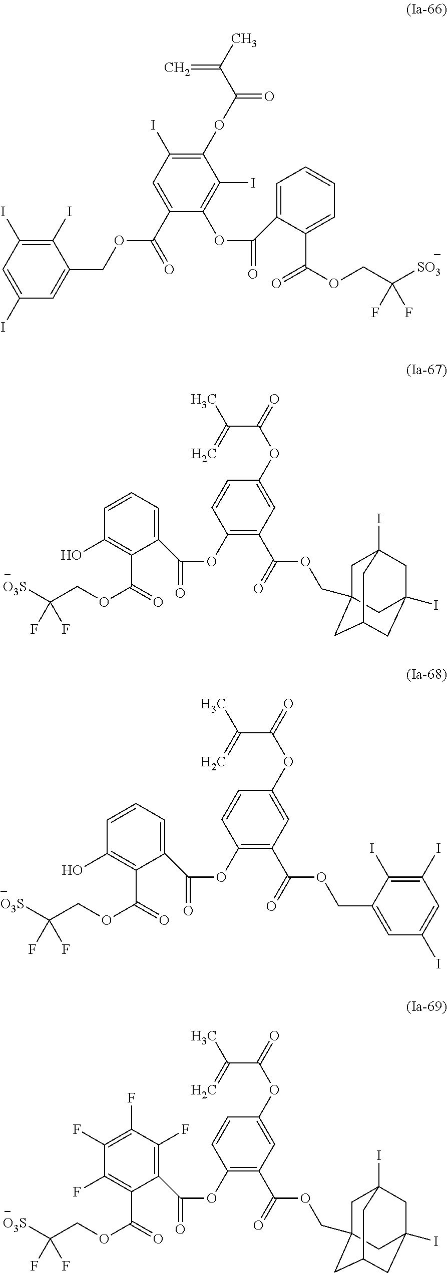

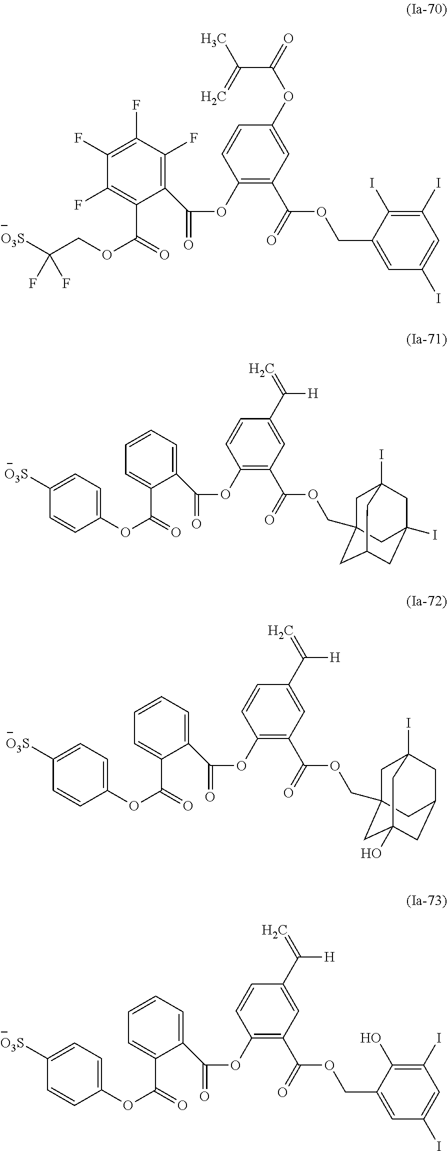

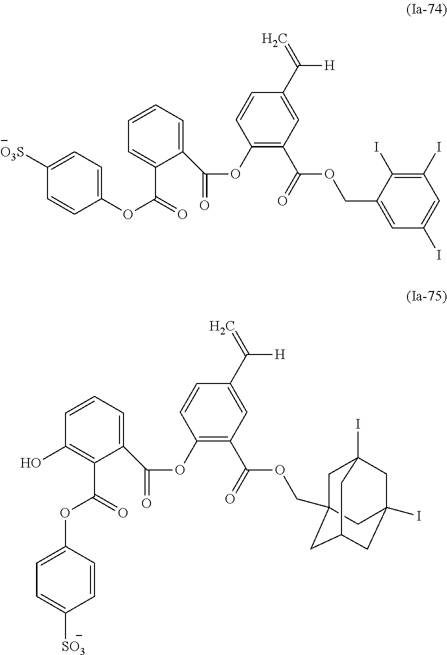

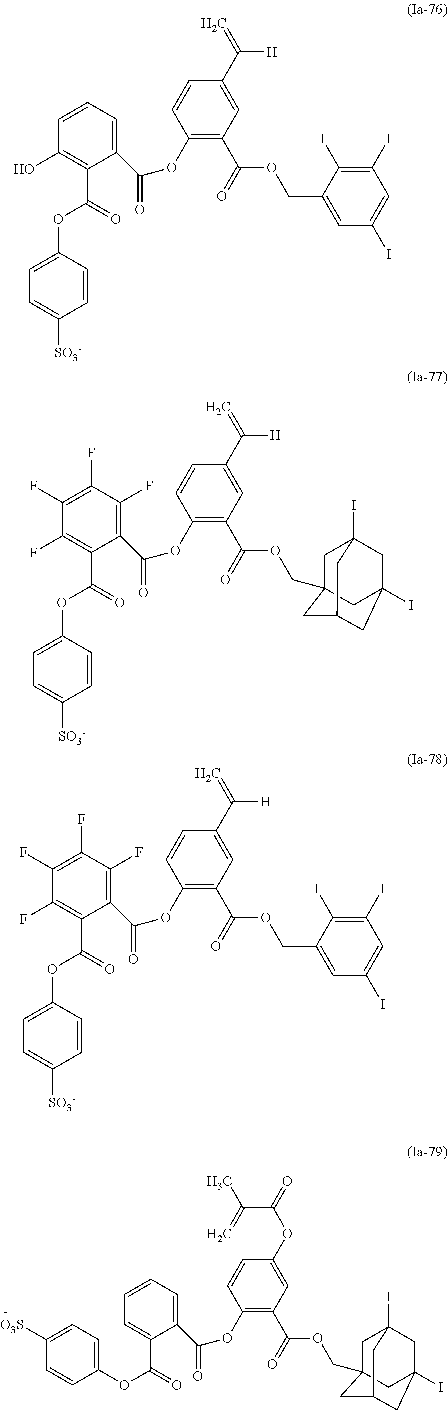

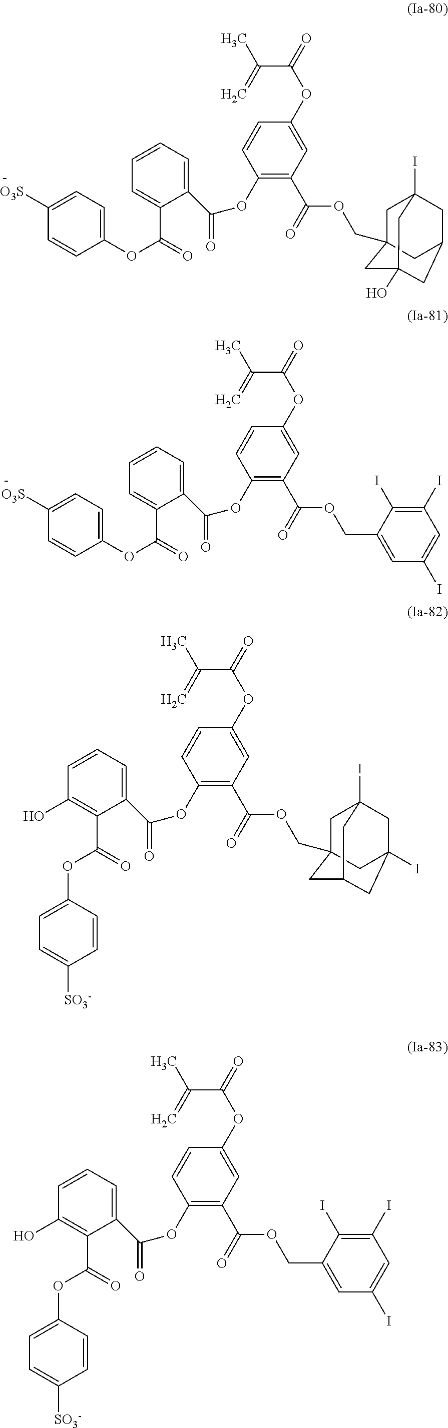

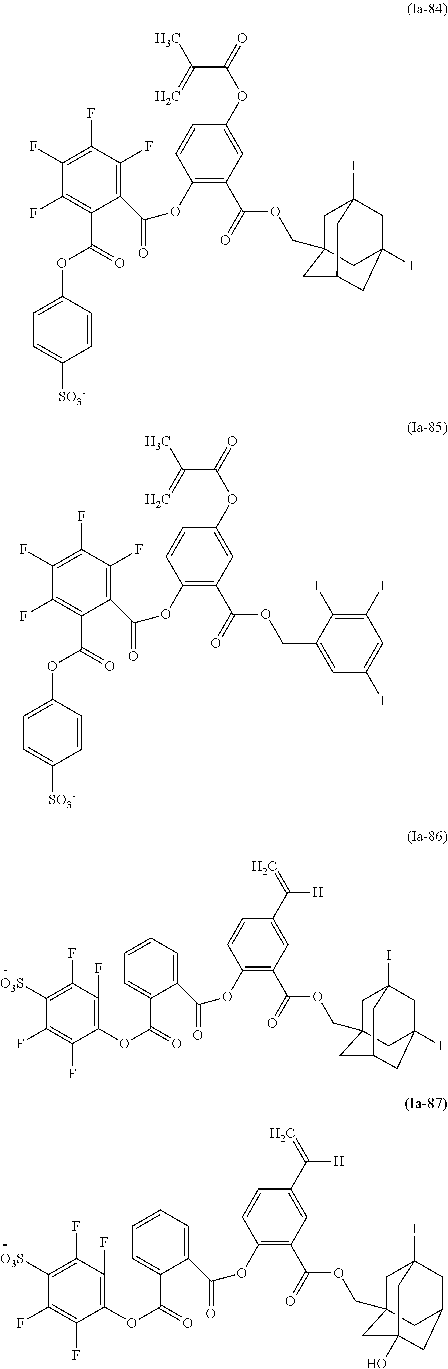

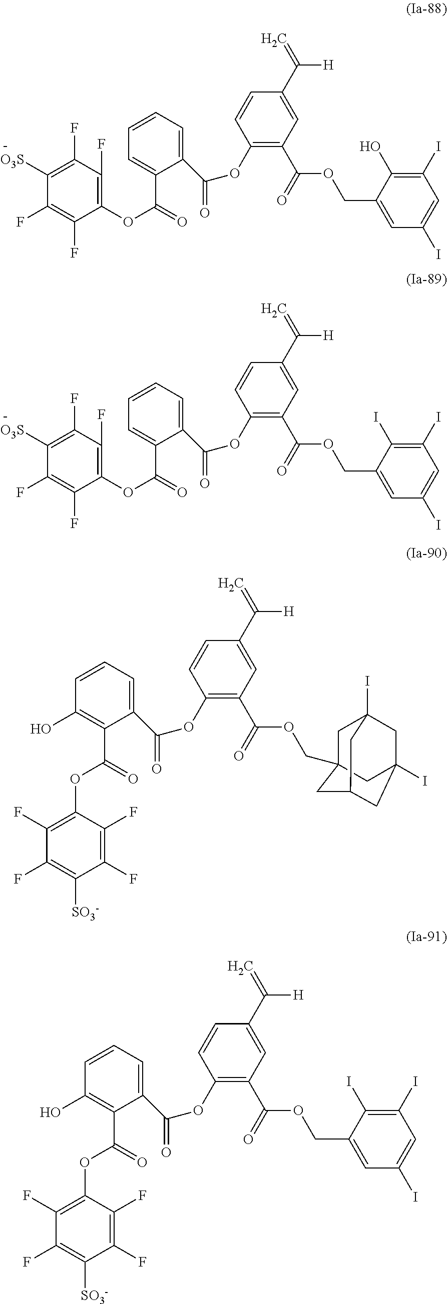

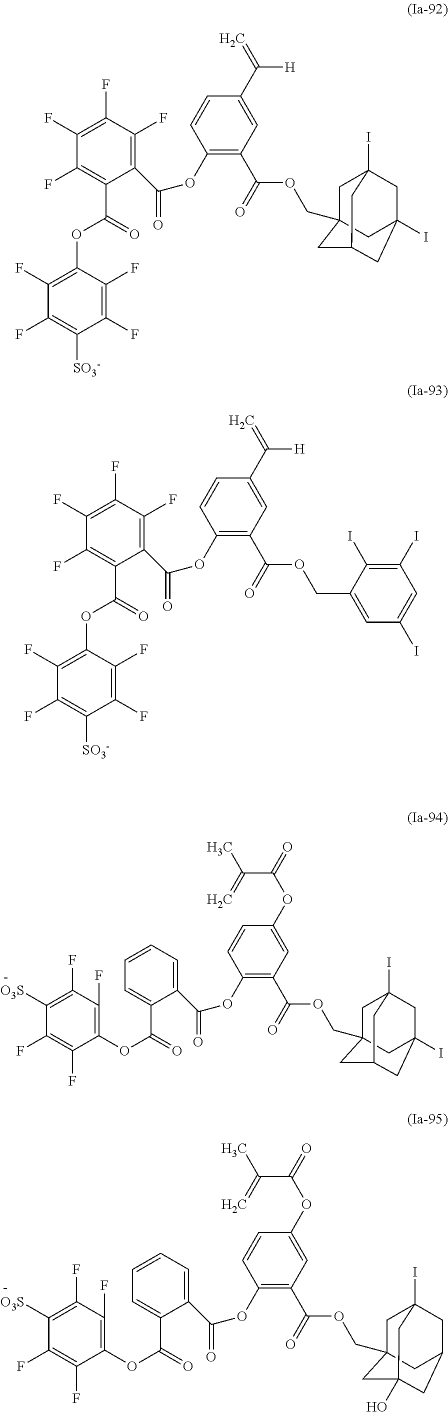

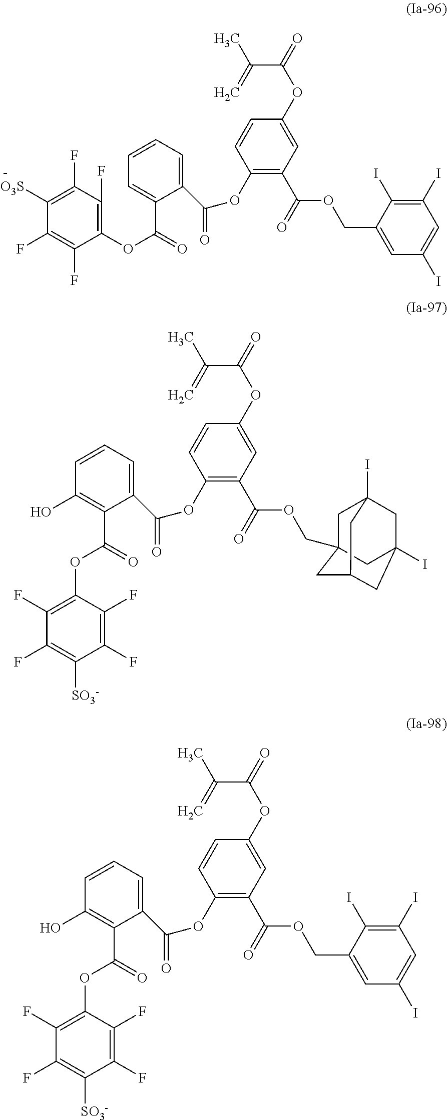

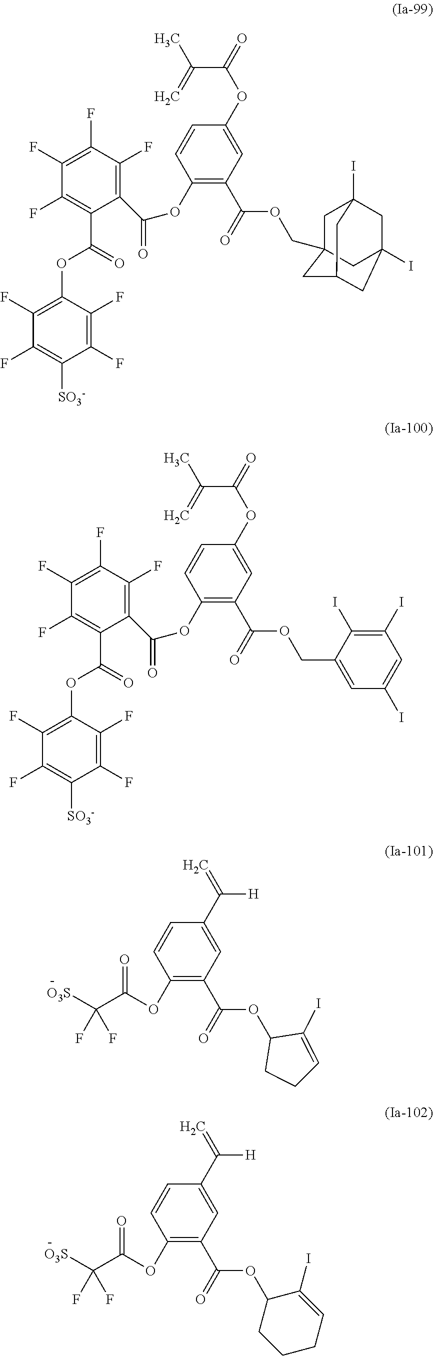

In the anions represented by the formulas (Ia-1) to (Ia-102), an anion in which a methyl group corresponding to R⁰ in the formula (I) is substituted with a hydrogen atom or the like, and an anion in which a hydrogen atom is substituted with a methyl group or the like can be provided as specific examples of the anion (I).

### [Cation (I)]

Examples of the organic cation as for Z⁺ in the formula (I) include an organic onium cation, an organic sulfonium cation, an organic iodonium cation, an organic ammonium cation, a benzothiazolium cation, and an organic phosphonium cation. Among these, an organic sulfonium cation and an organic iodonium cation are preferable, and an aryl sulfonium cation is more preferable. Specific examples thereof include a cation represented by any one of a formula (b2-1) to a formula (b2-5) (hereinafter may be referred to as “cation (b2-1)” or the like according to the number of formula).

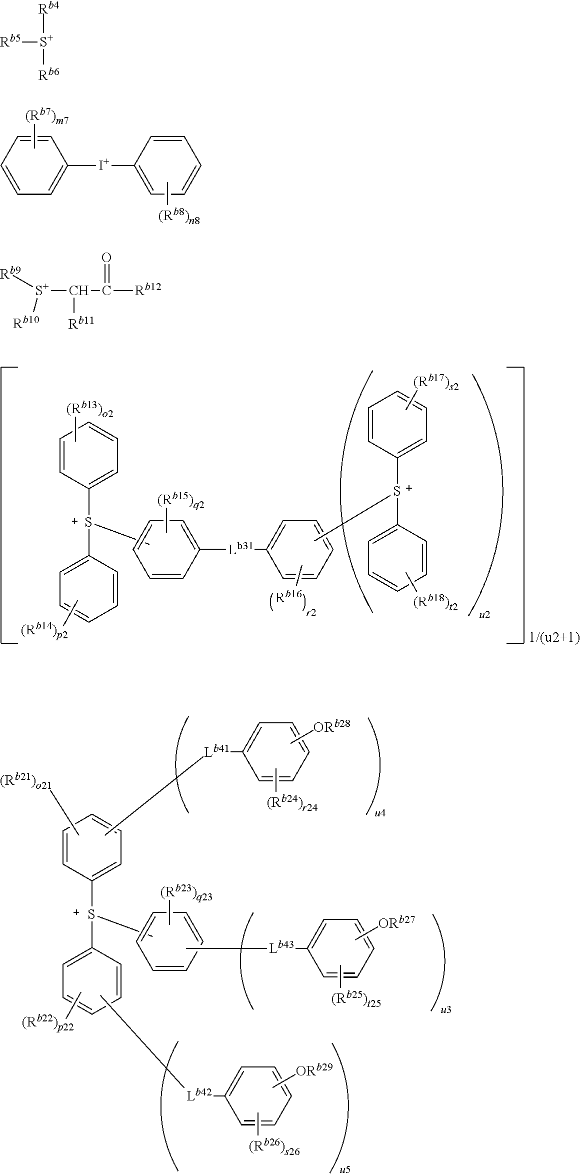

[In the formula (b2-1) to the formula (b2-5),

R^{b4} to R^{b6} each independently represent a chain hydrocarbon group having 1 to 30 carbon atoms, an alicyclic hydrocarbon group having 3 to 36 carbon atoms or an aromatic hydrocarbon group having 6 to 36 carbon atoms, a hydrogen atom included in the chain hydrocarbon group may be substituted with a hydroxy group, an alkoxy group having 1 to 12 carbon atoms, an alicyclic hydrocarbon group having 3 to 12 carbon atoms or an aromatic hydrocarbon group having 6 to 18 carbon atoms, a hydrogen atom included in the alicyclic hydrocarbon group may be substituted with a halogen atom, an aliphatic hydrocarbon group having 1 to 18 carbon atoms, an alkylcarbonyl group having 2 to 4 carbon atoms or a glycidyloxy group, and a hydrogen atom included in the aromatic hydrocarbon group may be substituted with a halogen atom, a hydroxy group, an aliphatic hydrocarbon group having 1 to 18 carbon atoms, an alkyl fluoride group having 1 to 12 carbon atoms or an alkoxy group having 1 to 12 carbon atoms.

R^{b4} and R^{b5} may be bonded to each other to form a ring together with sulfur atoms to which R^{b4} and R^{b5} are bonded, and —CH₂— included in the ring may be substituted with —O—, —S— or —CO—.

R^{b7} and R^{b8} each independently represent a halogen atom, a hydroxy group, an aliphatic hydrocarbon group having 1 to 12 carbon atoms, an alkyl fluoride group having 1 to 12 carbon atoms or an alkoxy group having 1 to 12 carbon atoms.

m7 and n8 each independently represent an integer of 0 to 5.

When m7 is 2 or more, a plurality of R^{b7} may be the same or different, and when n8 is 2 or more, a plurality of R^{b8} may be the same or different.

R^{b9} and R^{b10} each independently represent a chain hydrocarbon group having 1 to 36 carbon atoms or an alicyclic hydrocarbon group having 3 to 36 carbon atoms.

R^{b9} and R^{b10} may be bonded to each other to form a ring together with sulfur atoms to which R^{b9} and R^{b10} are bonded, and —CH₂— included in the ring may be substituted with —O—, —S— or —CO—.

R^{b11} represents a hydrogen atom, a chain hydrocarbon group having 1 to 36 carbon atoms, an alicyclic hydrocarbon group having 3 to 36 carbon atoms or an aromatic hydrocarbon group having 6 to 18 carbon atoms.

R^{b12} represents a chain hydrocarbon group having 1 to 12 carbon atoms, an alicyclic hydrocarbon group having 3 to 18 carbon atoms, or an aromatic hydrocarbon group having 6 to 18 carbon atoms, and a hydrogen atom included in the chain hydrocarbon group may be substituted with an aromatic hydrocarbon group having 6 to 18 carbon atoms, and a hydrogen atom included in the aromatic hydrocarbon group may be substituted with an alkoxy group having 1 to 12 carbon atoms or an alkylcarbonyloxy group having 1 to 12 carbon atoms.

R^{b11} and R^{b12} may be bonded to each other to form a ring, including —CH—CO— to which R^{b11} and R^{b12} are bonded, and —CH₂— included in the ring may be substituted with —O—, —S— or —CO—.

R^{b13} to R^{b18} and R^{b21} to R^{b22} each independently represent a halogen atom, a hydroxy group, an aliphatic hydrocarbon group having 1 to 12 carbon atoms, an alkyl fluoride group having 1 to 12 carbon atoms or an alkoxy group having 1 to 12 carbon atoms.

R^{b13} and R^{b14}, and R^{b21} and R^{b22} may be bonded to each other to form a ring having a sulfur atom together with the benzene ring to which R^{b13} and R^{b14} are bonded, and —CH₂— included in the ring may be substituted with —O—, —S—, or —CO—.

R^{b27} to R^{b29} each independently represent a hydrogen atom or a group having an acid-labile group.

L^{b31} and L^{b41} to L^{b43} each independently represent a sulfur atom or an oxygen atom.

o2, p2, s2, and t2 each independently represent an integer of 0 to 5.

q2 and r2 each independently represent an integer of 0 to 4.

u2 represents 0 or 1.

When o2 is 2 or more, a plurality of R^{b13} are the same or different, when p2 is 2 or more, a plurality of R^{b14} are the same or different, when q2 is 2 or more, a plurality of R^{b15} are the same or different, when r2 is 2 or more, a plurality of R^{b16} are the same or different, when s2 is 2 or more, a plurality of R^{b17} are the same or different, and when t2 is 2 or more, a plurality of R^{b18} are the same or different.

u3 represents an integer of 1 to 3, and satisfies 1≤u3+q23≤5.

u4 and u5 each independently represent an integer of 0 to 3, and satisfy 0≤u4+o21≤5 and 0≤u5+p22≤5.

When u3, u4, and u5 are each 2 or more, a plurality of groups in parentheses are each the same or different.

o21, p22, q23, r24, t25, and s26 each independently represent an integer of 0 to 4.

When o21, p22, q23, r24, t25, and s26 are each 2 or more, a plurality of R^{b21} to R^{b22} are each the same or different]

When u2 is 0, any one of o2, p2, q2, and r2 is preferably 1 or more and at least one of R^{b13} to R^{b16} is preferably a halogen atom, and when u2 is 1, any one of o2, p2, s2, t2, q2, and r2 is preferably 1 or more, and at least one of R^{b13} to R^{b18} is preferably a halogen atom.

Further, when u2 is 0, r2 is preferably 1 or more, and more preferably 1. In addition, when u2 is 0 and r2 is 1 or more, R^{b16} is preferably a halogen atom.

The aliphatic hydrocarbon group represents a chain hydrocarbon group and an alicyclic hydrocarbon group.

Examples of the chain hydrocarbon group include the same group as the alkyl group exemplified in the present specification. Particularly, the chain hydrocarbon group of R^{b9} to R^{b12} preferably has 1 to 12 carbon atoms.

The alicyclic hydrocarbon group may be either monocyclic or polycyclic, saturated or unsaturated, and examples thereof include the same group as the cycloalkyl group and the like exemplified in the present specification. Particularly, the alicyclic hydrocarbon group of R^{b9} to R^{b12} preferably has 3 to 18 carbon atoms, and more preferably 4 to 12 carbon atoms.

Examples of the alicyclic hydrocarbon group in which a hydrogen atom is substituted with an aliphatic hydrocarbon group include a methylcyclohexyl group, a dimethylcyclohexyl group, a 2-methyladamantan-2-yl group, a 2-ethyladamantan-2-yl group, a 2-isopropyladamantan-2-yl group, a methylnorbornyl group, and an isobornyl group. In the alicyclic hydrocarbon group in which a hydrogen atom is substituted with an aliphatic hydrocarbon group, the total number of carbon atoms of the alicyclic hydrocarbon group and the aliphatic hydrocarbon group is preferably 20 or less.

Examples of the fluorinated alkyl group include the same groups as those exemplified in the present specification. The number of carbon atoms of the fluorinated alkyl group is preferably 1 to 9, more preferably 1 to 6, still more preferably 1 to 4.

Examples of the aromatic hydrocarbon group include aryl groups such as a phenyl group, a biphenyl group, a naphthyl group and a phenanthryl group. The aromatic hydrocarbon group may have a chain hydrocarbon group or an alicyclic hydrocarbon group, and examples thereof include aromatic hydrocarbon groups which have a chain hydrocarbon group (a tolyl group, a xylyl group, a cumenyl group, a mesityl group, a p-ethylphenyl group, a p-tert-butylphenyl group, a 2,6-diethylphenyl group, a 2-methyl-6-ethylphenyl group, and the like) and aromatic hydrocarbon groups which have an alicyclic hydrocarbon group (a p-cyclohexylphenyl group, and a p-adamantylphenyl group and the like).

When the aromatic hydrocarbon group has a chain hydrocarbon group or an alicyclic hydrocarbon group, a chain hydrocarbon group having 1 to 18 carbon atoms and an alicyclic hydrocarbon group having 3 to 18 carbon atoms are preferable.

Examples of the aromatic hydrocarbon group in which a hydrogen atom is substituted with an alkoxy group include a p-methoxyphenyl group.

Examples of the chain hydrocarbon group in which a hydrogen atom is substituted with an aromatic hydrocarbon group include aralkyl groups such as a benzyl group, a phenethyl group, a phenylpropyl group, a trityl group, a naphthylmethyl group, and a naphthylethyl group.

Examples of the alkoxy group, the alkylcarbonyl group, the halogen atom, and the alkylcarbonyloxy group include the same groups as those exemplified in the present specification.

The ring formed by bonding R^{b4} and R^{b5} each other, together with sulfur atoms to which R^{b4} and R^{b5} are bonded, may be a monocyclic, polycyclic, aromatic, nonaromatic, saturated or unsaturated ring. This ring includes a ring having 3 to 18 carbon atoms and is preferably a ring having 4 to 18 carbon atoms. The ring having a sulfur atom includes a 3-membered to 12-membered ring and is preferably a 3-membered to 7-membered ring and includes, for example, the following rings. * represents a binding site.

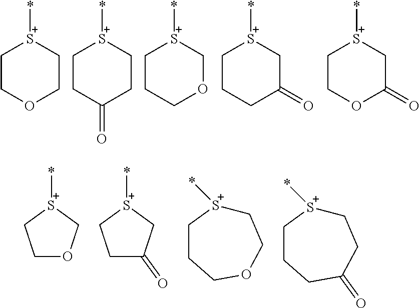

The ring formed by combining R^{b9} and R^{b10} together may be a monocyclic, polycyclic, aromatic, nonaromatic, saturated or unsaturated ring. This ring includes a 3-membered to 12-membered ring and is preferably a 3-membered to 7-membered ring. The ring includes, for example, a thiolan-1-ium ring (tetrahydrothiophenium ring), a thian-1-ium ring, a 1,4-oxathian-4-ium ring and the like.

The ring formed by combining R^{b11} and R^{b12} together may be a monocyclic, polycyclic, aromatic, nonaromatic, saturated or unsaturated ring. This ring includes a 3-membered to 12-membered ring and is preferably a 3-membered to 7-membered ring. Examples thereof include an oxocycloheptane ring, an oxocyclohexane ring, an oxonorbornane ring, and an oxoadamantane ring.

Examples of the group having an acid-labile group as for R^{b27} to R^{b29} include a group represented by —R^{c30}, —CO—O—R^{c30}, or -L^{c10}-CO—O—R^{c30} (L^{c10} represents an alkanediyl group having 1 to 6 carbon atoms, and R^{c30} represents an acid-labile group).

Examples of the acid-labile group of R^{c30} include acid-labile groups represented by a formula (1) or a formula (2) exemplified herein.

Among a cation (b2-1) to a cation (b2-5), the cation (b2-1), the cation (b2-4), or the cation (b2-5) are preferable.

Examples of the cation (b2-1) include the following cations.

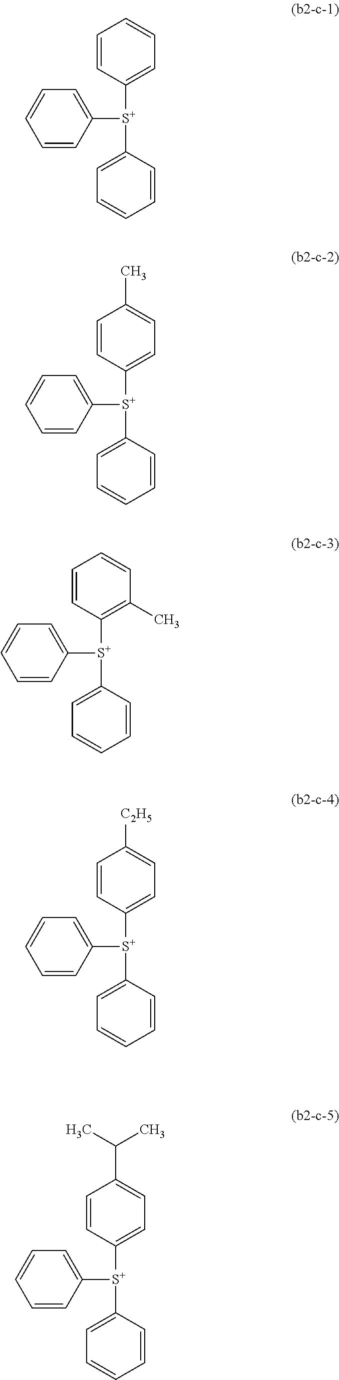 [SMILES: c1ccc([S+](c2ccccc2)c2ccccc2)cc1 ; Cc1ccc([S+](c2ccccc2)c2ccccc2)cc1 ; Cc1ccccc1[S+](c1ccccc1)c1ccccc1 ; CCc1ccc([S+](c2ccccc2)c2ccccc2)cc1 ; CC(C)c1ccc([S+](c2ccccc2)c2ccccc2)cc1]

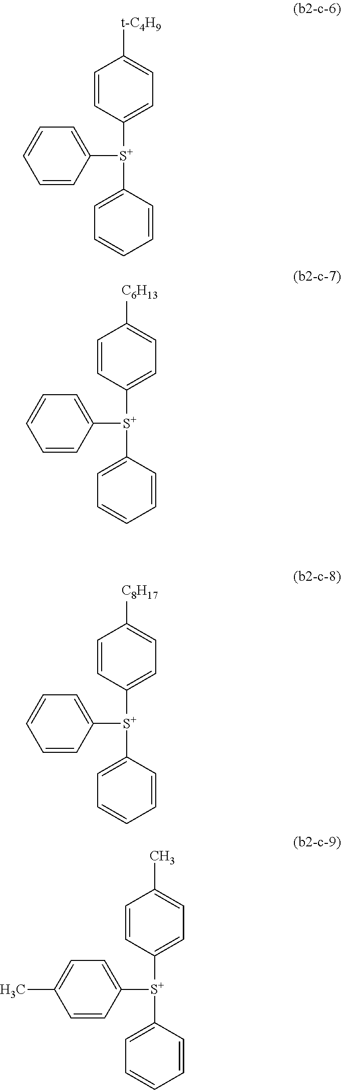

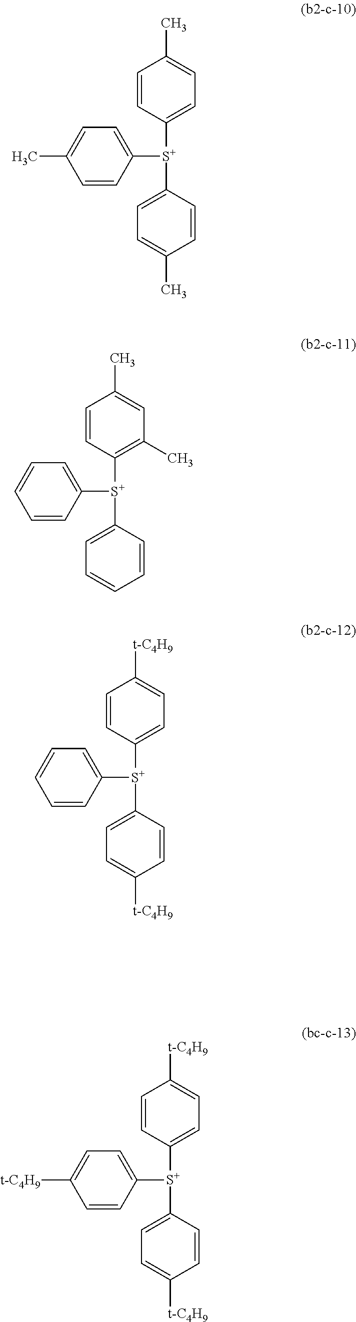

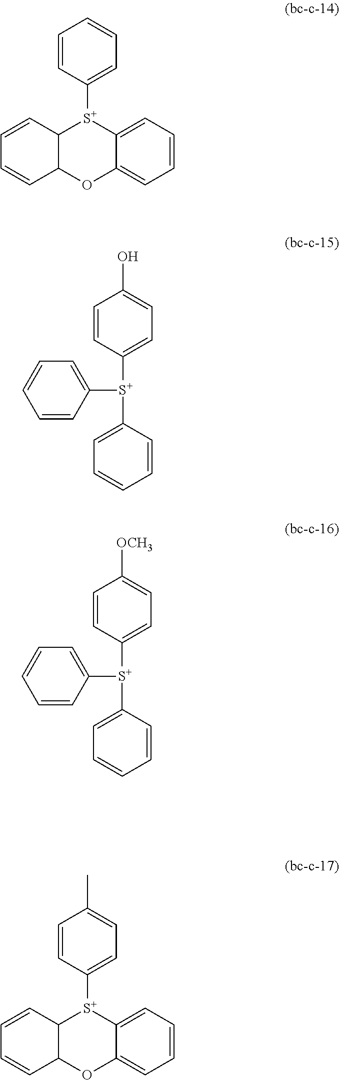 [SMILES: C1=CC2Oc3ccccc3[S+](c3ccccc3)C2C=C1 ; Oc1ccc([S+](c2ccccc2)c2ccccc2)cc1 ; COc1ccc([S+](c2ccccc2)c2ccccc2)cc1 ; Cc1ccc([S+]2c3ccccc3OC3C=CC=CC32)cc1]

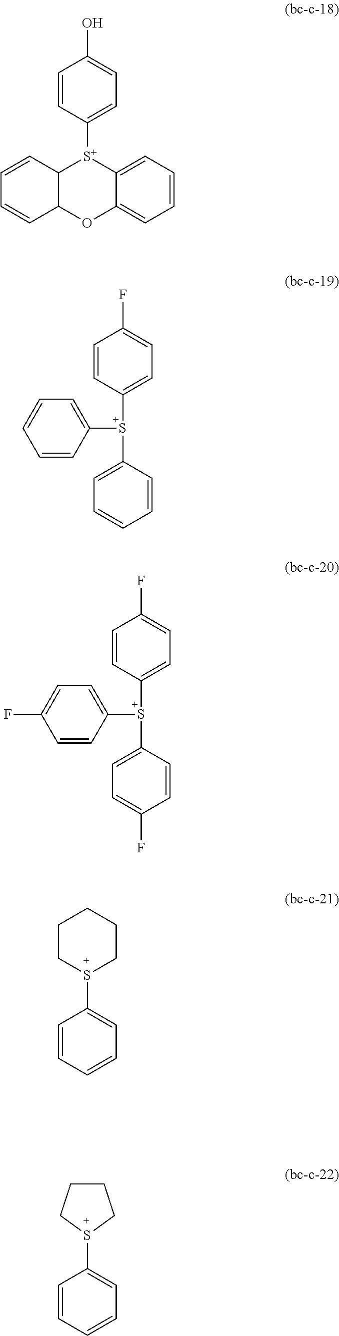

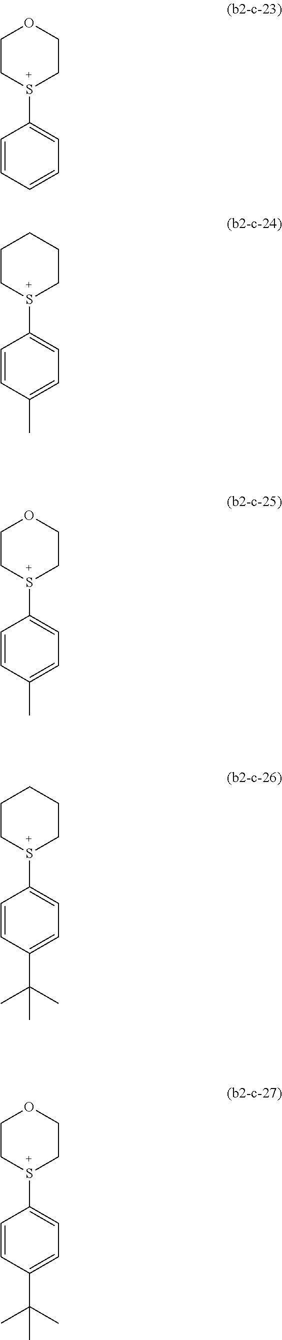

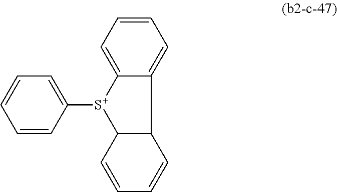 [SMILES: C1=CC2c3ccccc3[S+](c3ccccc3)C2C=C1]

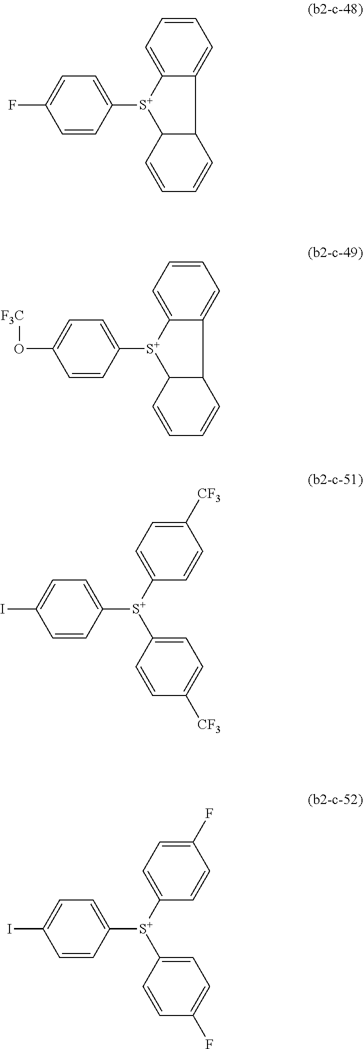 [SMILES: Fc1ccc([S+]2c3ccccc3C3C=CC=CC32)cc1 ; FC(F)(F)Oc1ccc([S+]2c3ccccc3C3C=CC=CC32)cc1 ; FC(F)(F)c1ccc([S+](c2ccc(I)cc2)c2ccc(C(F)(F)F)cc2)cc1 ; Fc1ccc([S+](c2ccc(F)cc2)c2ccc(I)cc2)cc1]

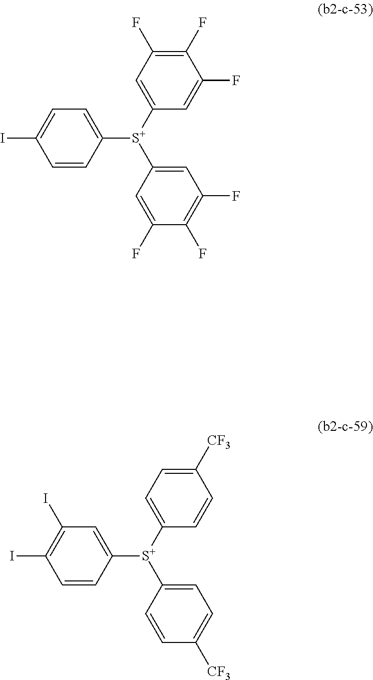 [SMILES: Fc1cc([S+](c2ccc(I)cc2)c2cc(F)c(F)c(F)c2)cc(F)c1F ; FC(F)(F)c1ccc([S+](c2ccc(C(F)(F)F)cc2)c2ccc(I)c(I)c2)cc1]

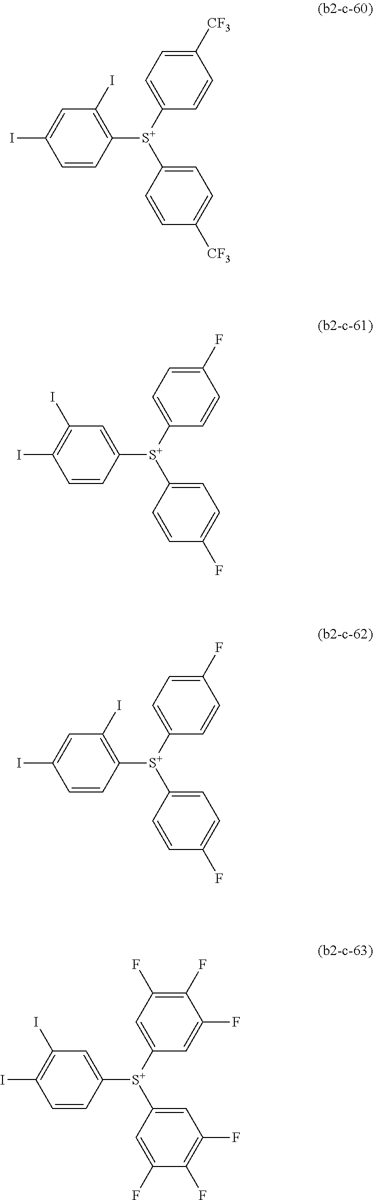 [SMILES: FC(F)(F)c1ccc([S+](c2ccc(C(F)(F)F)cc2)c2ccc(I)cc2I)cc1 ; Fc1ccc([S+](c2ccc(F)cc2)c2ccc(I)c(I)c2)cc1 ; Fc1ccc([S+](c2ccc(F)cc2)c2ccc(I)cc2I)cc1 ; Fc1cc([S+](c2cc(F)c(F)c(F)c2)c2ccc(I)c(I)c2)cc(F)c1F]

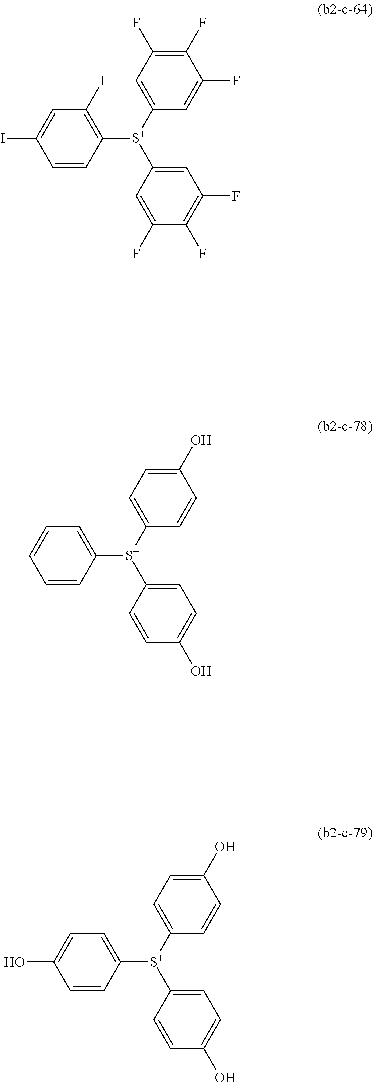 [SMILES: Fc1cc([S+](c2cc(F)c(F)c(F)c2)c2ccc(I)cc2I)cc(F)c1F ; Oc1ccc([S+](c2ccccc2)c2ccc(O)cc2)cc1 ; Oc1ccc([S+](c2ccc(O)cc2)c2ccc(O)cc2)cc1]

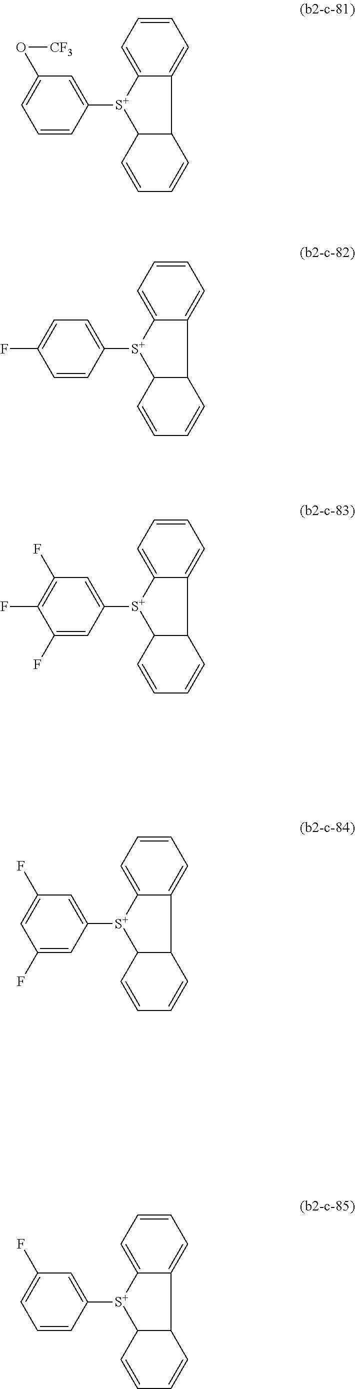 [SMILES: FC(F)(F)Oc1cccc([S+]2c3ccccc3C3C=CC=CC32)c1 ; Fc1ccc([S+]2c3ccccc3C3C=CC=CC32)cc1 ; Fc1cc([S+]2c3ccccc3C3C=CC=CC32)cc(F)c1F ; Fc1cc(F)cc([S+]2c3ccccc3C3C=CC=CC32)c1 ; Fc1cccc([S+]2c3ccccc3C3C=CC=CC32)c1]

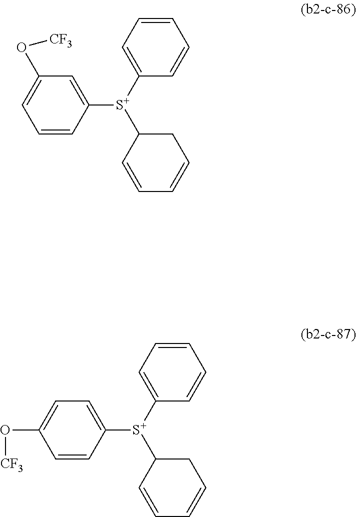 [SMILES: FC(F)(F)Oc1cccc([S+](c2ccccc2)C2C=CC=CC2)c1 ; FC(F)(F)Oc1ccc([S+](c2ccccc2)C2C=CC=CC2)cc1]

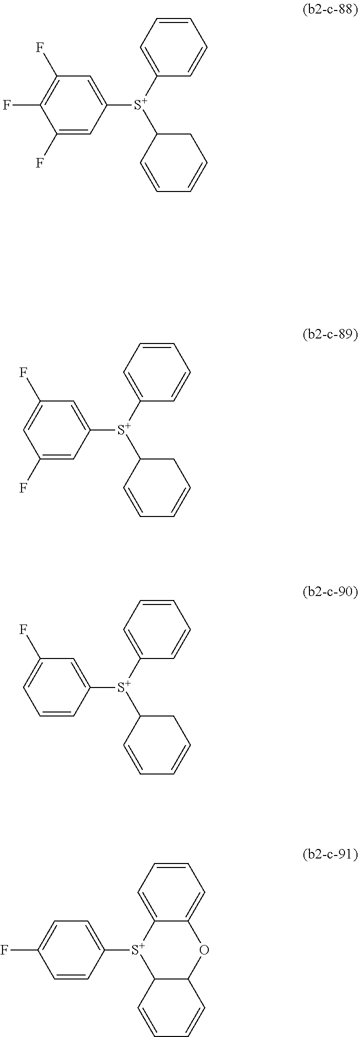 [SMILES: Fc1cc([S+](c2ccccc2)C2C=CC=CC2)cc(F)c1F ; Fc1cc(F)cc([S+](c2ccccc2)C2C=CC=CC2)c1 ; Fc1cccc([S+](c2ccccc2)C2C=CC=CC2)c1 ; Fc1ccc([S+]2c3ccccc3OC3C=CC=CC32)cc1]

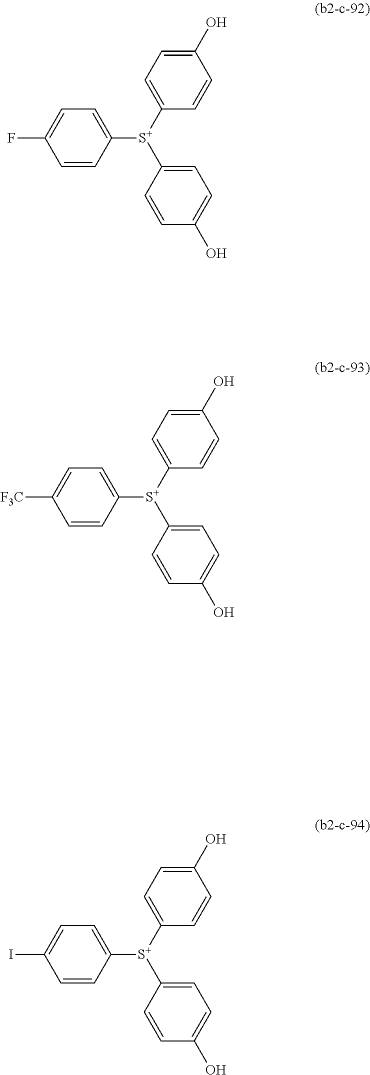 [SMILES: Oc1ccc([S+](c2ccc(O)cc2)c2ccc(F)cc2)cc1 ; Oc1ccc([S+](c2ccc(O)cc2)c2ccc(C(F)(F)F)cc2)cc1 ; Oc1ccc([S+](c2ccc(O)cc2)c2ccc(I)cc2)cc1]

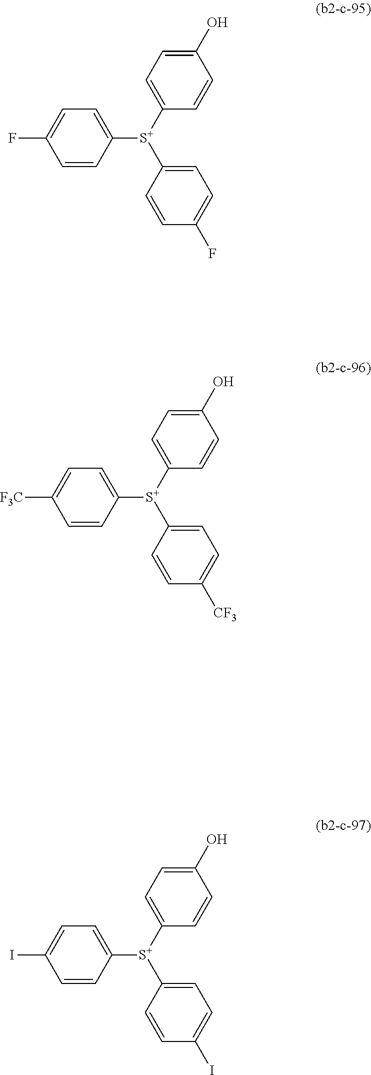 [SMILES: Oc1ccc([S+](c2ccc(F)cc2)c2ccc(F)cc2)cc1 ; Oc1ccc([S+](c2ccc(C(F)(F)F)cc2)c2ccc(C(F)(F)F)cc2)cc1 ; Oc1ccc([S+](c2ccc(I)cc2)c2ccc(I)cc2)cc1]

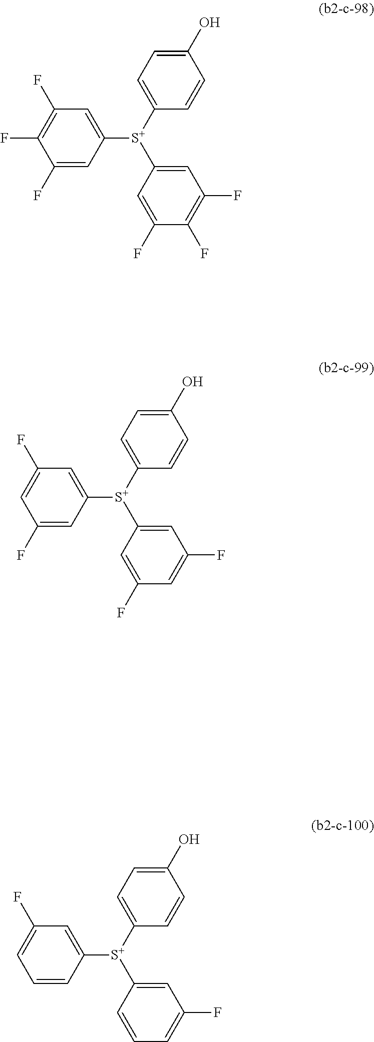 [SMILES: Oc1ccc([S+](c2cc(F)c(F)c(F)c2)c2cc(F)c(F)c(F)c2)cc1 ; Oc1ccc([S+](c2cc(F)cc(F)c2)c2cc(F)cc(F)c2)cc1 ; Oc1ccc([S+](c2cccc(F)c2)c2cccc(F)c2)cc1]

Examples of the cation (b2-2) include the following cations.

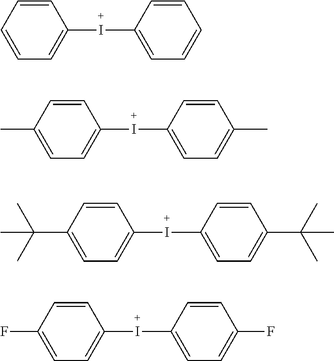

Examples of the cation (b2-3) include the following cations.

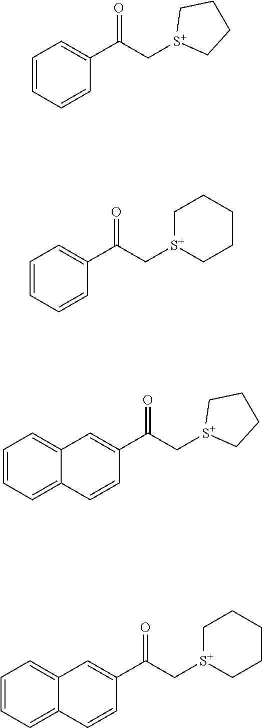 [SMILES: O=C(C[S+]1CCCC1)c1ccccc1 ; O=C(C[S+]1CCCCC1)c1ccccc1 ; O=C(C[S+]1CCCC1)c1ccc2ccccc2c1 ; O=C(C[S+]1CCCCC1)c1ccc2ccccc2c1]

Examples of the cation (b2-4) include the following cations.

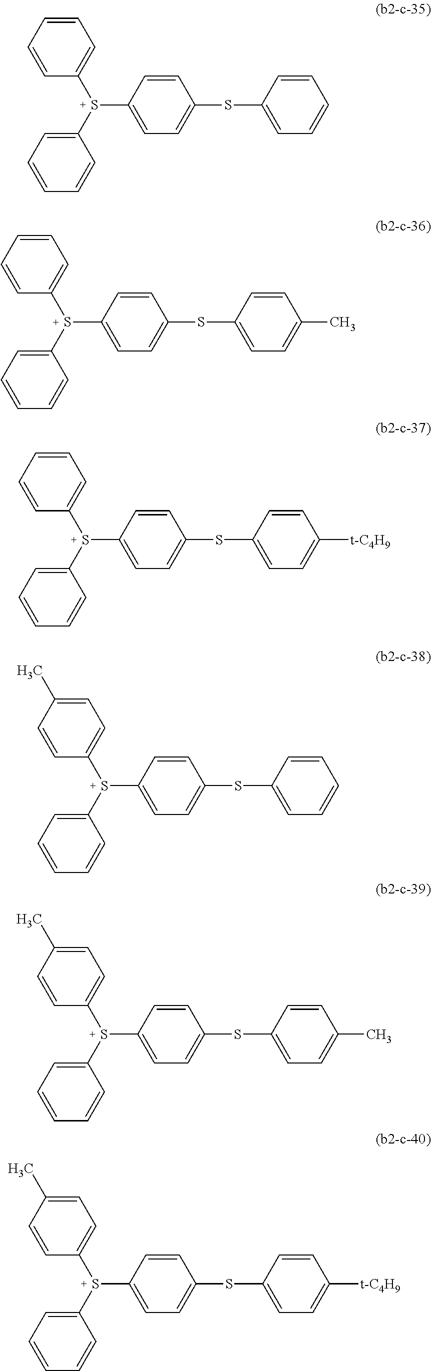

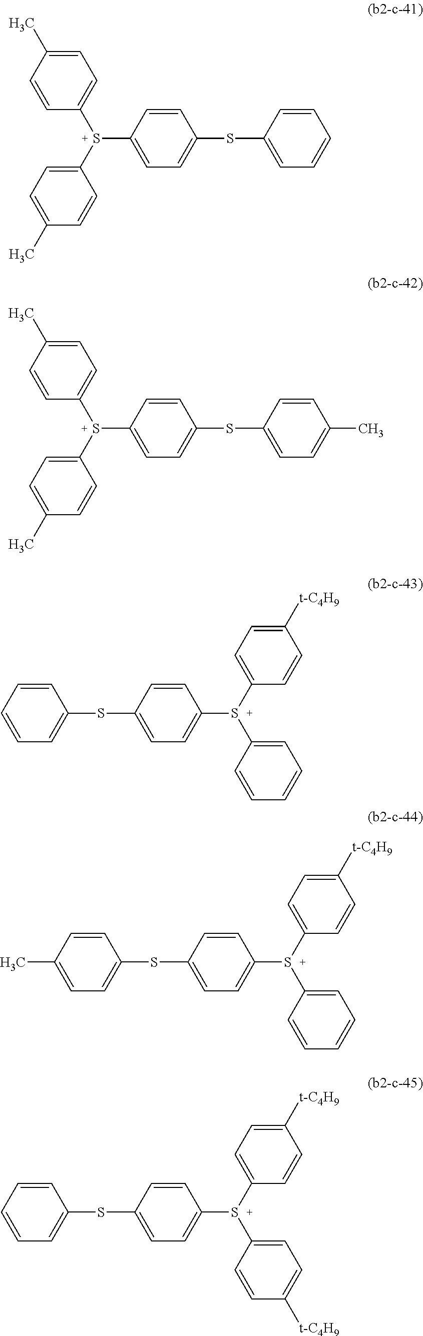

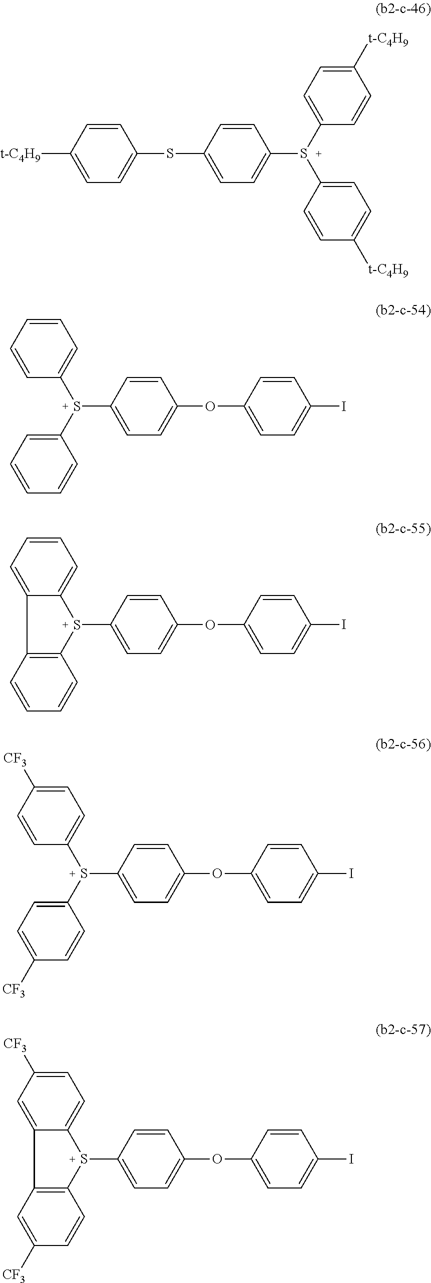

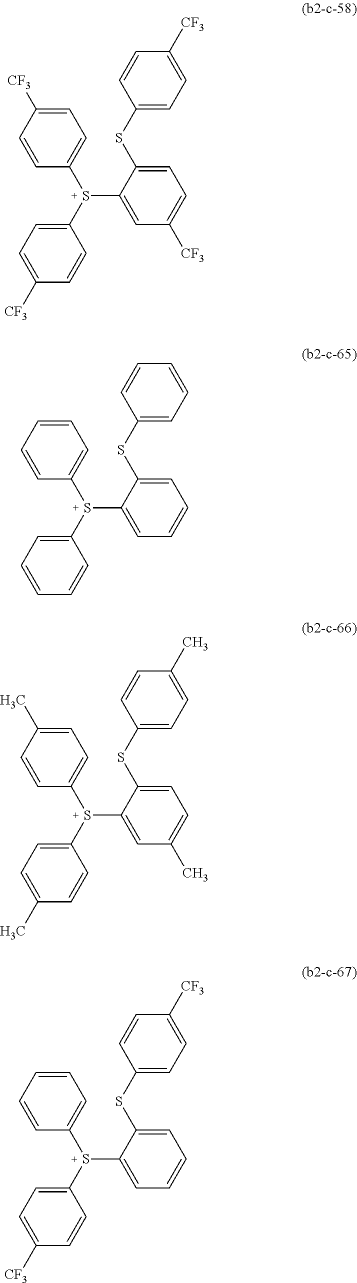

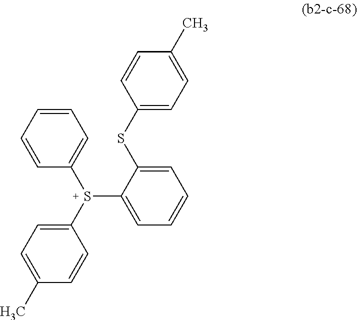

Examples of the cation (b2-5) include the following cations.

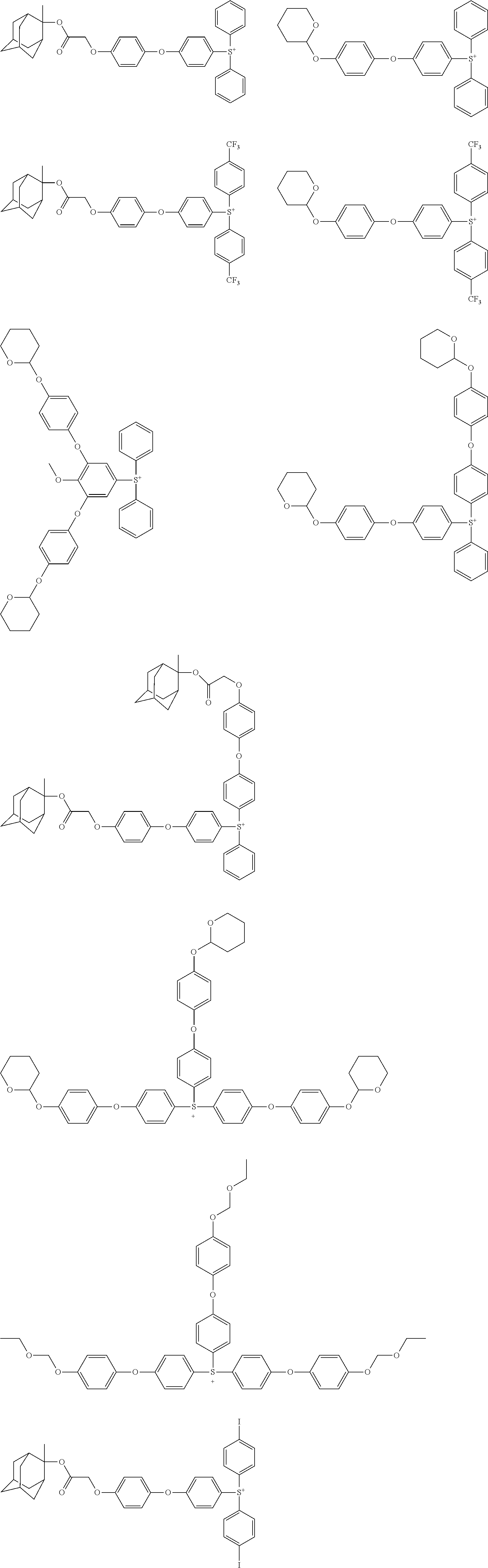

Specific examples of the salt (I) include salts obtained by optionally combining the above-described cations and anions. Specific examples of the salt (I) are shown in the following table.

In the following table, the respective reference numerals represent reference numerals imparted to structures showing the above-described anions and cations, and “~” represents that the salt (I) and the anion (I) correspond, respectively. For example, the salt (I-1) is a salt composed of an anion represented by a formula (Ia-1) and a cation represented by a formula (b2-c-1), the salt (I-2) is a salt composed of an anion represented by a formula (Ia-2) and a cation represented by a formula (b2-c-1), and the salt (I-103) is a salt composed of an anion represented by the formula (Ia-1) and a cation represented by the formula (b2-c-10).

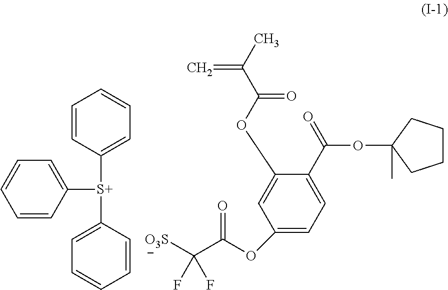

TABLE 1

| Salt (I) | Anion (I) | Cation (I) |
|---|---|---|
| (I-1) to (I-102) | (Ia-1) to (Ia-102) | (b2-c-1) |
| (I-103) to (I-204) | (Ia-1) to (Ia-102) | (b2-c-10) |
| (I-205) to (I-306) | (Ia-1) to (Ia-102) | (b2-c-13) |
| (I-307) to (I-408) | (Ia-1) to (Ia-102) | (b2-c-14) |
| (I-409) to (I-510) | (Ia-1) to (Ia-102) | (b2-c-18) |
| (I-511) to (I-612) | (Ia-1) to (Ia-102) | (b2-c-19) |
| (I-613) to (I-714) | (Ia-1) to (Ia-102) | (b2-c-20) |
| (I-715) to (I-816) | (Ia-1) to (Ia-102) | (b2-c-27) |
| (I-817) to (I-918) | (Ia-1) to (Ia-102) | (b2-c-30) |
| (I-919) to (I-1020) | (Ia-1) to (Ia-102) | (b2-c-31) |
| (I-1021) to (I-1122) | (Ia-1) to (Ia-102) | (b2-c-50) |
| (I-1123) to (I-1224) | (Ia-1) to (Ia-102) | (b2-c-51) |
| (I-1225) to (I-1326) | (Ia-1) to (Ia-102) | (b2-c-54) |
| (I-1327) to (I-1428) | (Ia-1) to (Ia-102) | (b2-c-55) |
| (I-1429) to (I-1530) | (Ia-1) to (Ia-102) | (b2-c-56) |
| (I-1531) to (I-1632) | (Ia-1) to (Ia-102) | (b2-c-57) |
| (I-1633) to (I-1734) | (Ia-1) to (Ia-102) | (b2-c-58) |
| (I-1735) to (I-1836) | (Ia-1) to (Ia-102) | (b2-c-59) |
| (I-1837) to (I-1938) | (Ia-1) to (Ia-102) | (b2-c-60) |
| (I-1939) to (I-2040) | (Ia-1) to (Ia-102) | (b2-c-61) |
| (I-2041) to (I-2142) | (Ia-1) to (Ia-102) | (b2-c-62) |
| (I-2143) to (I-2244) | (Ia-1) to (Ia-102) | (b2-c-63) |
| (I-2245) to (I-2346) | (Ia-1) to (Ia-102) | (b2-c-64) |
| (I-2347) to (I-2448) | (Ia-1) to (Ia-102) | (b2-c-65) |
| (I-2449) to (I-2550) | (Ia-1) to (Ia-102) | (b2-c-66) |
| (I-2551) to (I-2652) | (Ia-1) to (Ia-102) | (b2-c-67) |
| (I-2653) to (I-2754) | (Ia-1) to (Ia-102) | (b2-c-68) |
| (I-2755) to (I-2856) | (Ia-1) to (Ia-102) | (b2-c-69) |
| (I-2857) to (I-2958) | (Ia-1) to (Ia-102) | (b2-c-70) |
| (I-2959) to (I-3060) | (Ia-1) to (Ia-102) | (b2-c-71) |
| (I-3061) to (I-3162) | (Ia-1) to (Ia-102) | (b2-c-72) |
| (I-3163) to (I-3264) | (Ia-1) to (Ia-102) | (b2-c-73) |
| (I-3265) to (I-3366) | (Ia-1) to (Ia-102) | (b2-c-74) |
| (I-3367) to (I-3468) | (Ia-1) to (Ia-102) | (b2-c-75) |
| (I-3469) to (I-3570) | (Ia-1) to (Ia-102) | (b2-c-76) |
| (I-3571) to (I-3672) | (Ia-1) to (Ia-102) | (b2-c-77) |
| (I-3673) to (I-3774) | (Ia-1) to (Ia-102) | (b2-c-78) |
| (I-3775) to (I-3876) | (Ia-1) to (Ia-102) | (b2-c-79) |

Among these, the salt (I) is preferably a salt obtained by combining an anion represented by any one of the formula (Ia-11) to formula (Ia-102) with a cation represented by any one of a formula (b2-c-1), a formula (b2-c-10), a formula (b2-c-13), a formula (b2-c-14), a formula (b2-c-18) to a formula (b2-c-20), a formula (b2-c-27), a formula (b2-c-30), a formula (b2-c-31), a formula (b2-c-50), a formula (b2-c-51), and a formula (b2-c-54) to a formula (b2-c-79).

### <Method for Producing Salt (I)>

The salt (I) can be obtained by reacting a salt represented by the formula (I-A) with a compound represented by the formula (I-B) in the presence of a catalyst in a solvent.

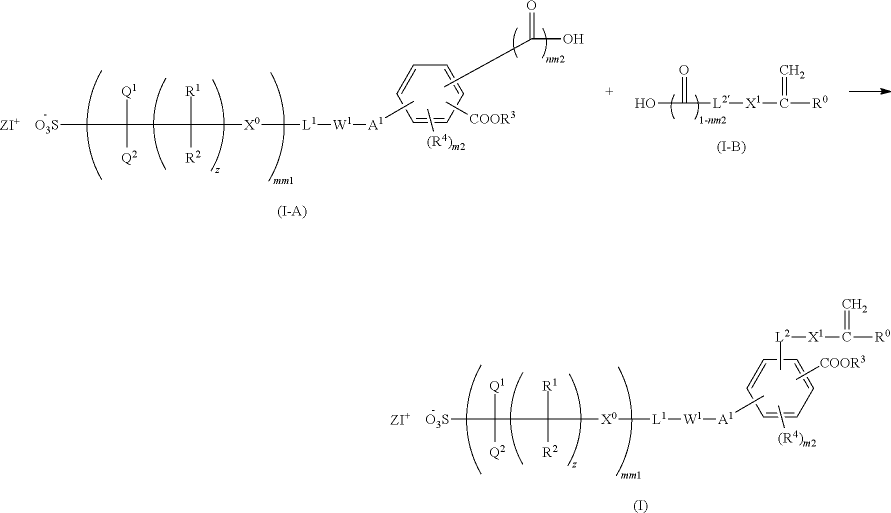

(In the formula, reference numerals other than the following each represent the same meaning as described above. nm2 represents 0 or 1. L^{2′} represents a group obtained by removing —(CO)ₙₘ₂—O—(CO)_{1·nm2}— from L²)

Examples of the solvent in this reaction include tetrahydrofuran, chloroform, and acetonitrile.

Examples of the catalyst include triethylamine, diisopropylethylamine, pyridine, dimethylaminopyridine, carbonyldiimidazole, sodium hydroxide, potassium hydroxide, and 1-[3-(dimethylamino)propyl]-3-ethylcarbodiimide hydrochloride.

The reaction temperature is typically 0° C. to 120° C., and the reaction timc is typically 0.5 hour to 24 hours.

Examples of the compound represented by formula (I-B) include compounds represented by the following formulas and the like and are easily available on the market, and can also be easily produced by a known production method.

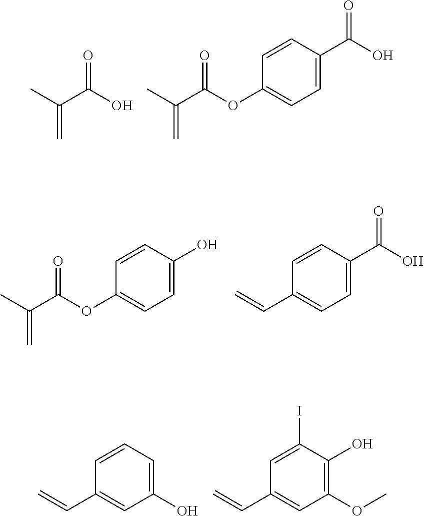

The salt (I-A) can be produced, for example, by reacting a salt represented by the formula (I-a1), a salt represented by the formula (I-a2), or a salt represented by the formula (I-a3) with a compound represented by the formula (I-b) in a solvent in the presence of a catalyst, and then subjecting the mixture to an acid treatment, as necessary.

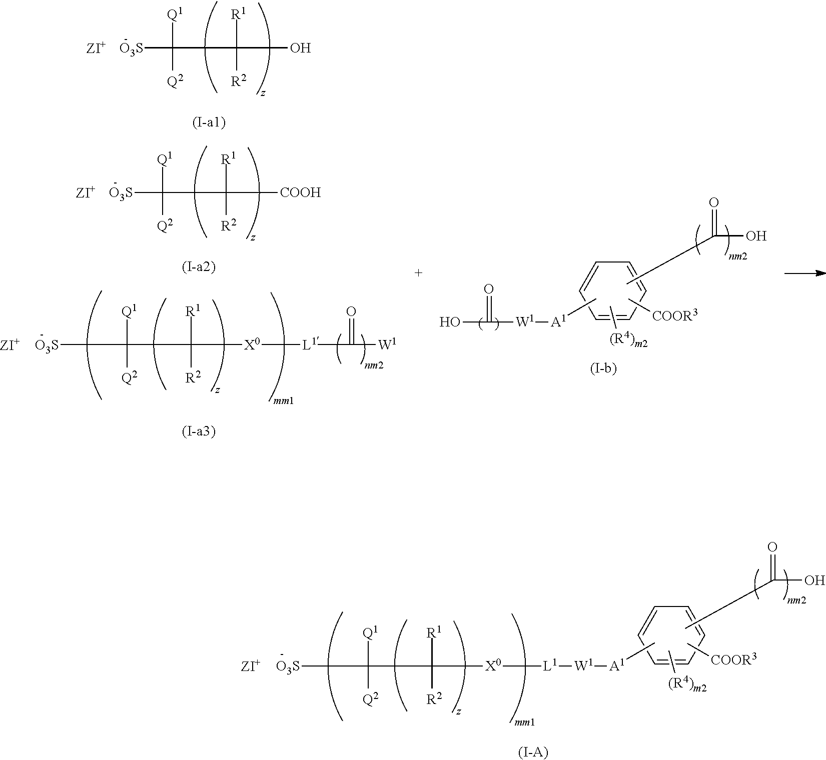

(In the formula, reference numerals other than the following each represent the same meaning as described above. nm2 represents 0 or 1. L^{1′} represents a group obtained by removing —(CO)ₙₘ₂O—(CO)₁₋ₙₘ₂ from L¹)

Examples of the solvent in this reaction include chloroform and acetonitrile.

Examples of the catalyst include carbonyldiimidazole and 1-[3-(dimethylamino) propyl]-3-ethylcarbodiimide hydrochloride.

Examples of the acid in this reaction include hydrochloric acid.

The reaction temperature is typically 5° C. to 80° C., and the reaction timc is typically 0.5 hour to 24 hours.

Examples of the salt represented by the formula (I-a1) include salts represented by the following formulas and are easily available on the market, and can also be easily produced by a known production method.

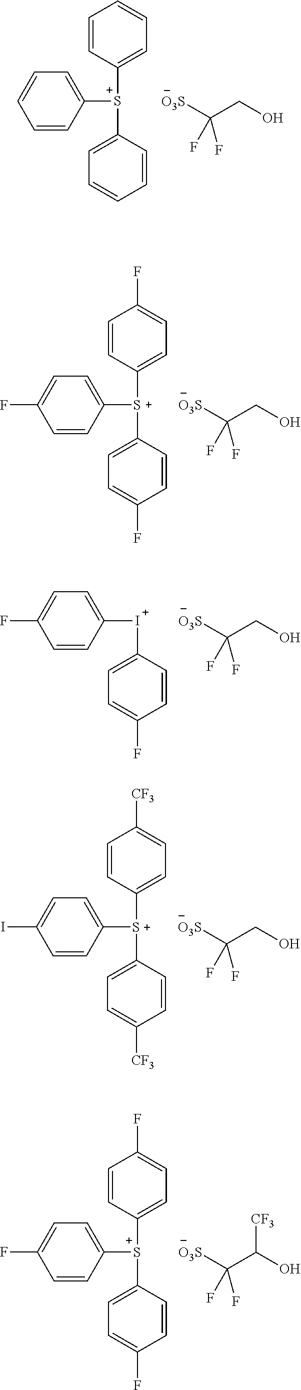

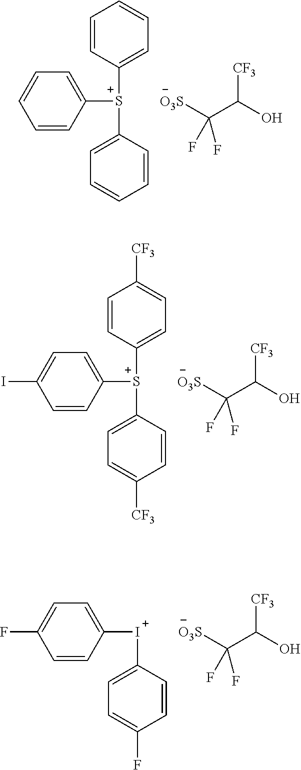

Examples of the salt represented by the formula (I-a2) include salts represented by the following formulas and are easily available on the market, and can also be easily produced by a known production method.

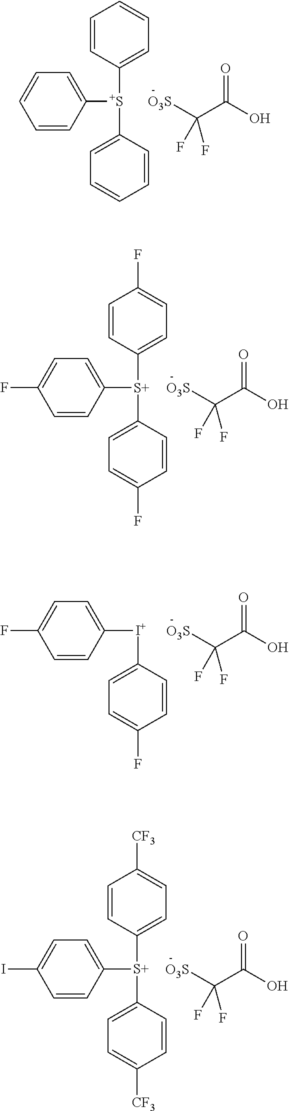

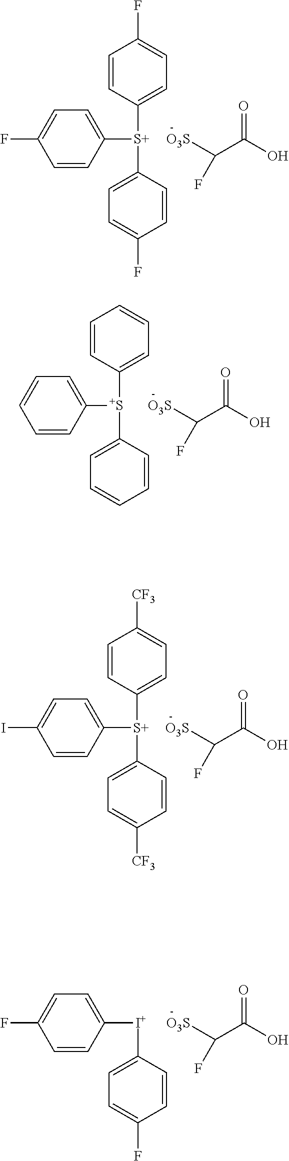

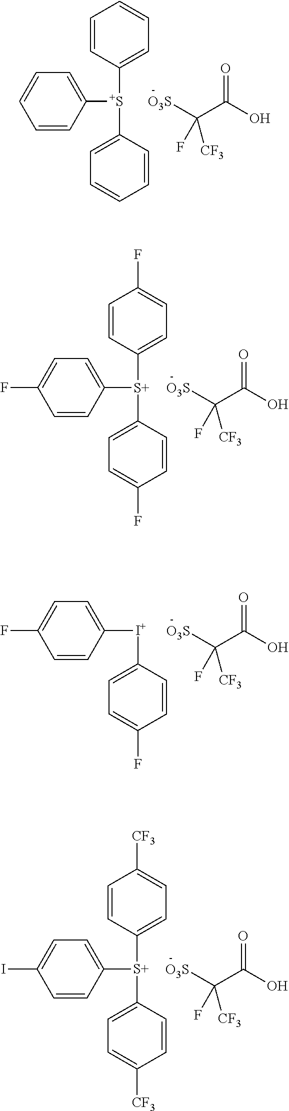

Examples of the salt represented by the formula (I-a3) include salts represented by the following formulas and are easily available on the market, and can also be easily produced by a known production method.

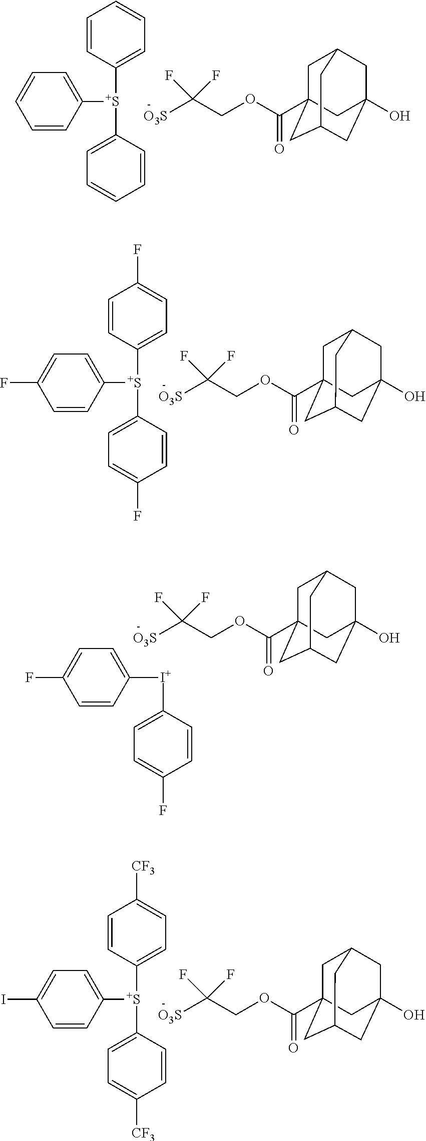

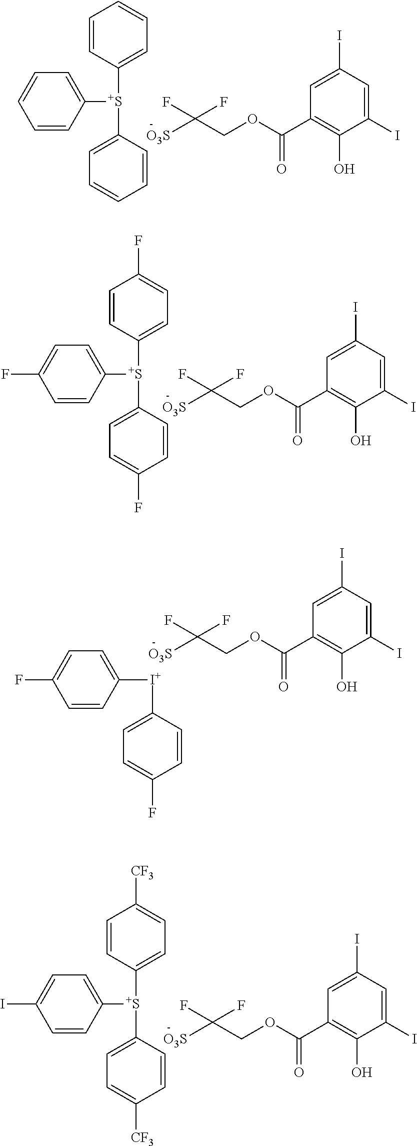

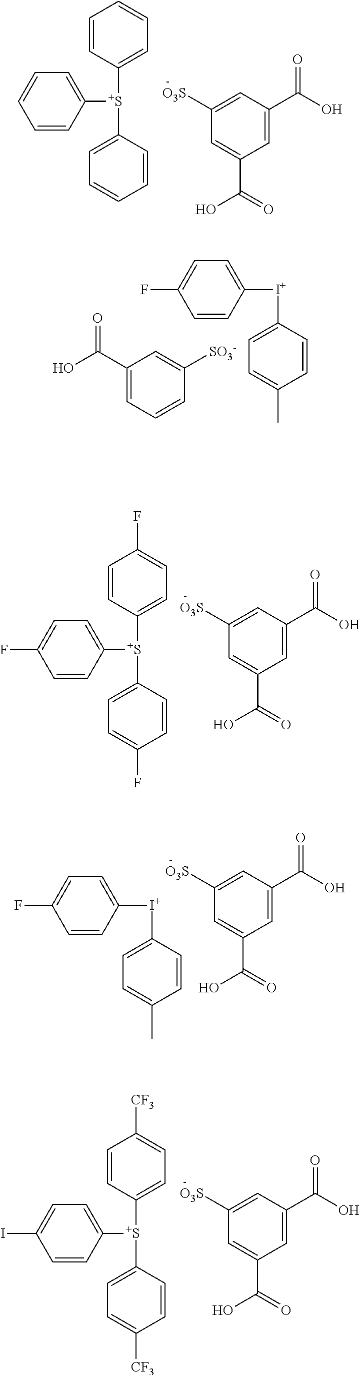

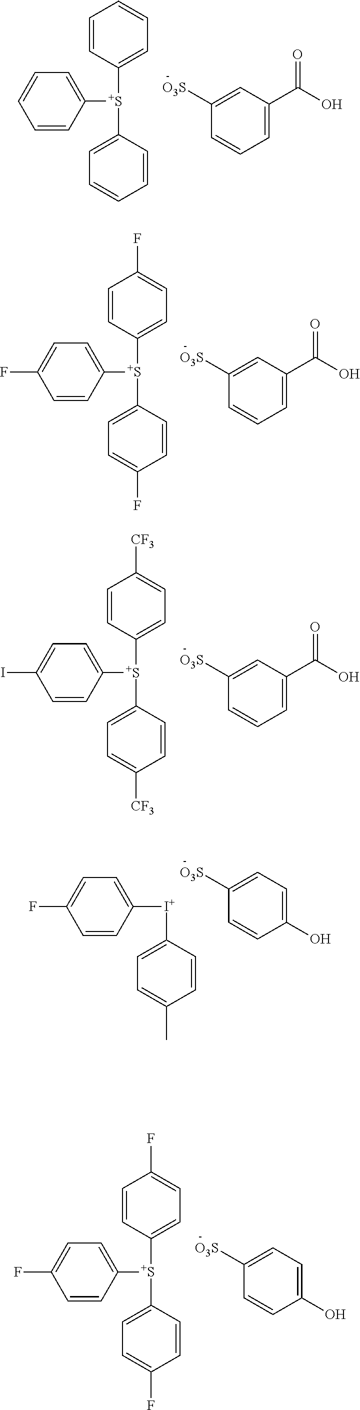

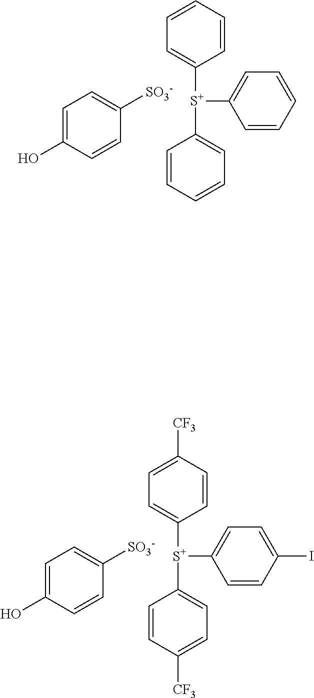

Examples of the compound represented by the formula (I-b) include compounds represented by the following formulas and the like, are easily available, and can be easily produced by a known production method.

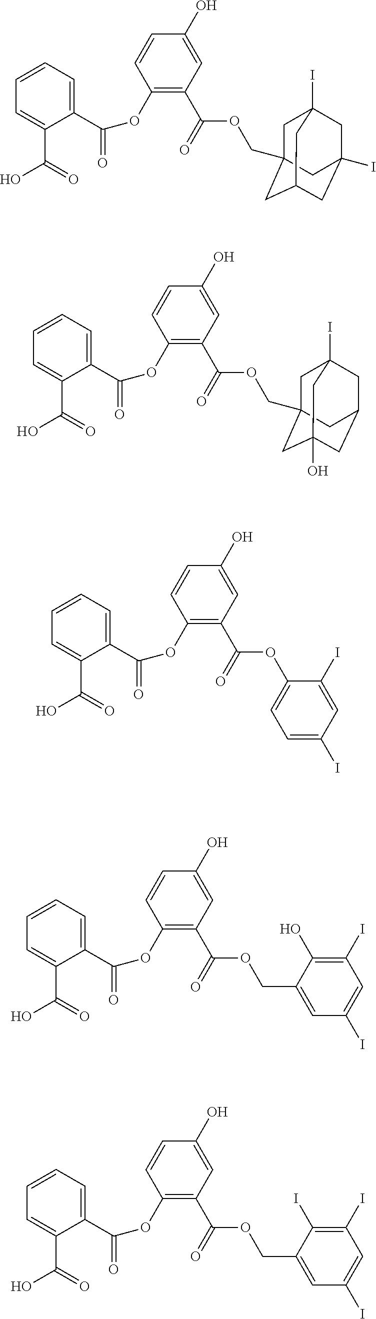 [SMILES: O=C(OCC12CC3CC(I)(CC(I)(C3)C1)C2)c1cc(O)ccc1OC(=O)c1ccccc1C(=O)O ; O=C(OCC12CC3CC(O)(CC(I)(C3)C1)C2)c1cc(O)ccc1OC(=O)c1ccccc1C(=O)O ; O=C(Oc1ccc(I)cc1I)c1cc(O)ccc1OC(=O)c1ccccc1C(=O)O ; O=C(OCc1cc(I)cc(I)c1O)c1cc(O)ccc1OC(=O)c1ccccc1C(=O)O ; O=C(OCc1cc(I)cc(I)c1I)c1cc(O)ccc1OC(=O)c1ccccc1C(=O)O]

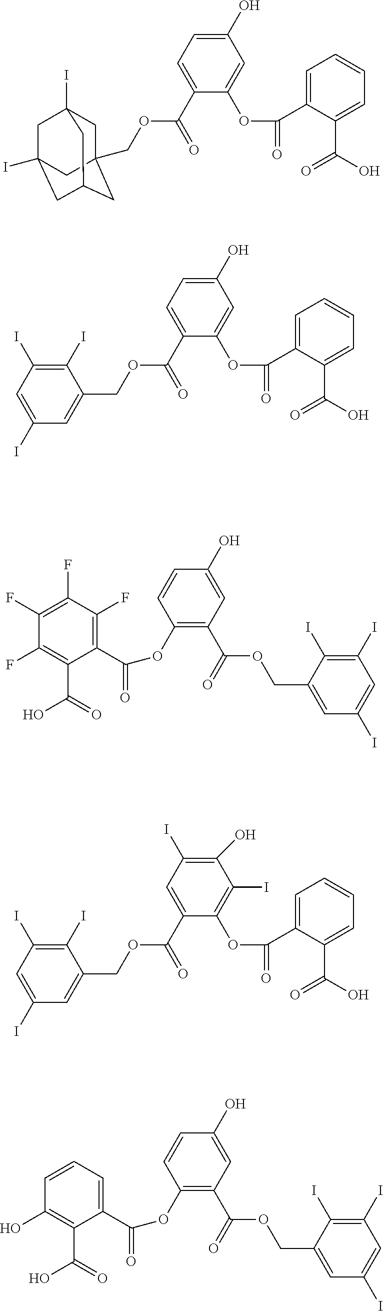 [SMILES: O=C(OCC12CC3CC(I)(CC(I)(C3)C1)C2)c1ccc(O)cc1OC(=O)c1ccccc1C(=O)O ; O=C(OCc1cc(I)cc(I)c1I)c1ccc(O)cc1OC(=O)c1ccccc1C(=O)O ; O=C(OCc1cc(I)cc(I)c1I)c1cc(O)ccc1OC(=O)c1c(F)c(F)c(F)c(F)c1C(=O)O ; O=C(O)c1ccccc1C(=O)Oc1c(C(=O)OCc2cc(I)cc(I)c2I)cc(I)c(O)c1I ; O=C(OCc1cc(I)cc(I)c1I)c1cc(O)ccc1OC(=O)c1cccc(O)c1C(=O)O]

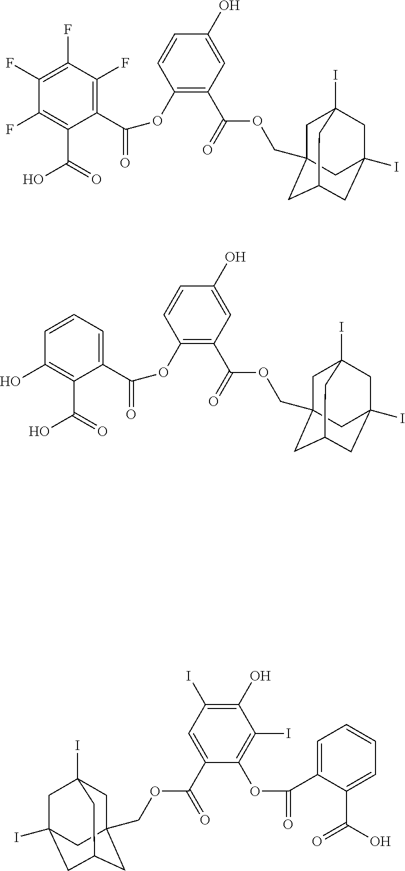 [SMILES: O=C(OCC12CC3CC(I)(CC(I)(C3)C1)C2)c1cc(O)ccc1OC(=O)c1c(F)c(F)c(F)c(F)c1C(=O)O ; O=C(OCC12CC3CC(I)(CC(I)(C3)C1)C2)c1cc(O)ccc1OC(=O)c1cccc(O)c1C(=O)O ; O=C(O)c1ccccc1C(=O)Oc1c(C(=O)OCC23CC4CC(I)(CC(I)(C4)C2)C3)cc(I)c(O)c1I]

The salt (I) can also be produced, for example, by reacting a salt represented by the formula (I-a1), a salt represented by the formula (I-a2), or a salt represented by the formula (I-a3) with a compound represented by the formula (I-C) in a solvent in the presence of a catalyst, and then subjecting the mixture to an acid treatment, as necessary.

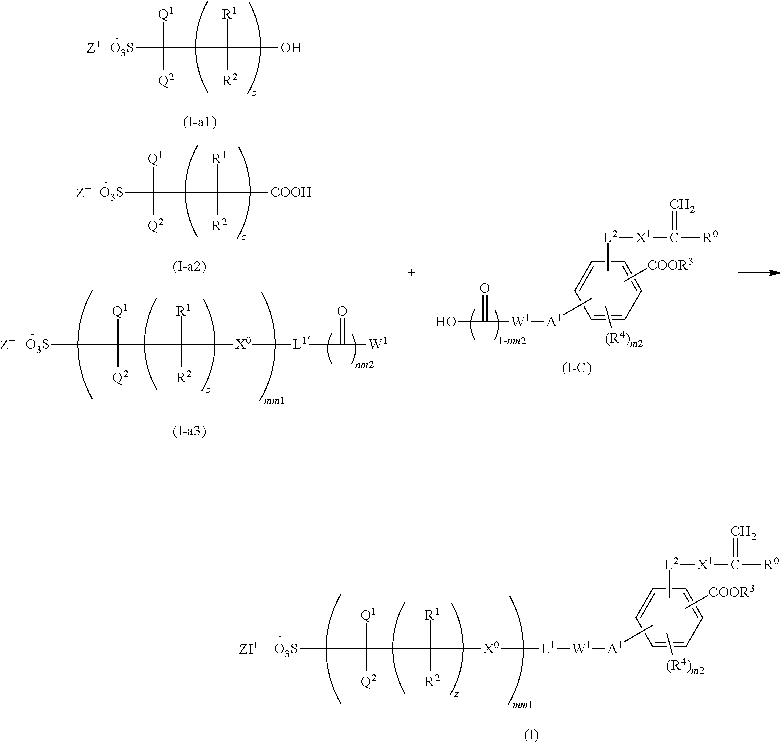

(In the formula, reference numerals other than the following each represent the same meaning as described above. nm2 represents 0 or 1. L^{1′} represents a group obtained by removing —(CO)ₙₘ₂—O—(CO)_{1·nm2}— from L¹)

Examples of the solvent in this reaction include chloroform and acetonitrile.

Examples of the catalyst include carbonyldiimidazole and 1-[3-(dimethylamino) propyl]-3-ethylcarbodiimide hydrochloride.

Examples of the acid in this reaction include hydrochloric acid.

The reaction temperature is typically 5° C. to 80° C., and the reaction timc is typically 0.5 hour to 24 hours.

A compound in which X¹ and L² are a single bond in a compound (I-C) (hereinafter, may be referred to as a compound (I0-C)) can be obtained by reacting a compound represented by the formula (I0-a), methyltriphenylphosphonium bromide, and potassium t-butoxide in a solvent.

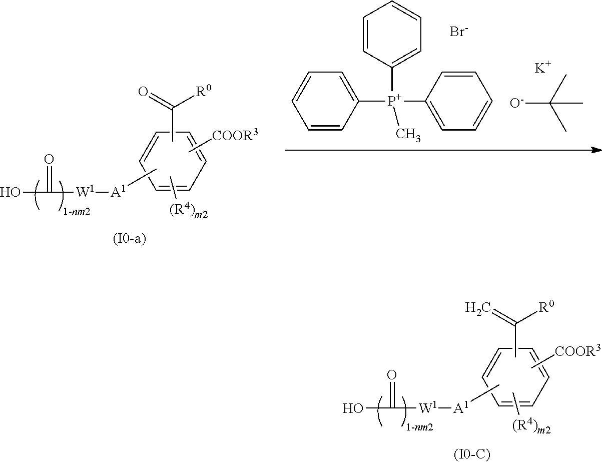

(In the formula, all reference numerals are the same as defined above)

Examples of the solvent include tetrahydrofuran and N,N-dimethylformamide.

The reaction temperature is usually 0° C. to 60° C., and the reaction timc is usually 0.5 hour to 24 hours.

Examples of the compound represented by the formula (I0-a) include a compound represented by the following formula, and the compound can be easily produced by a known production method.

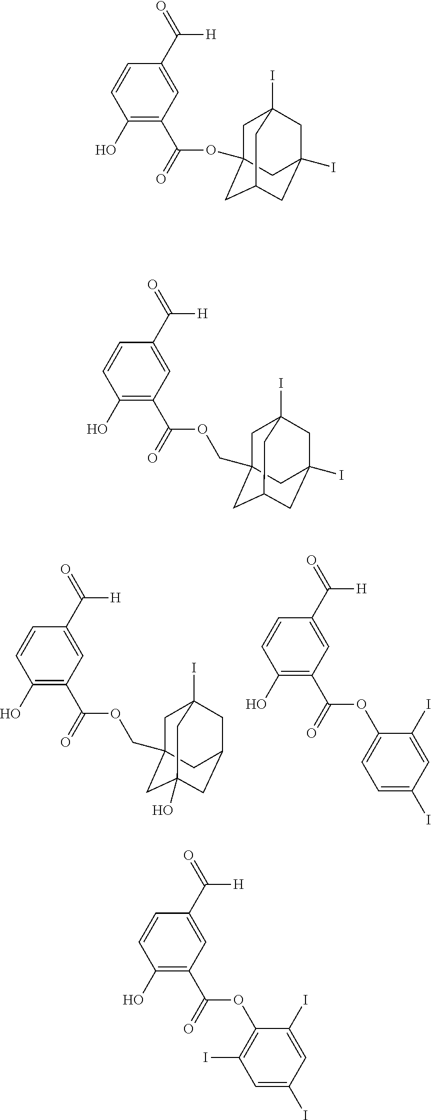

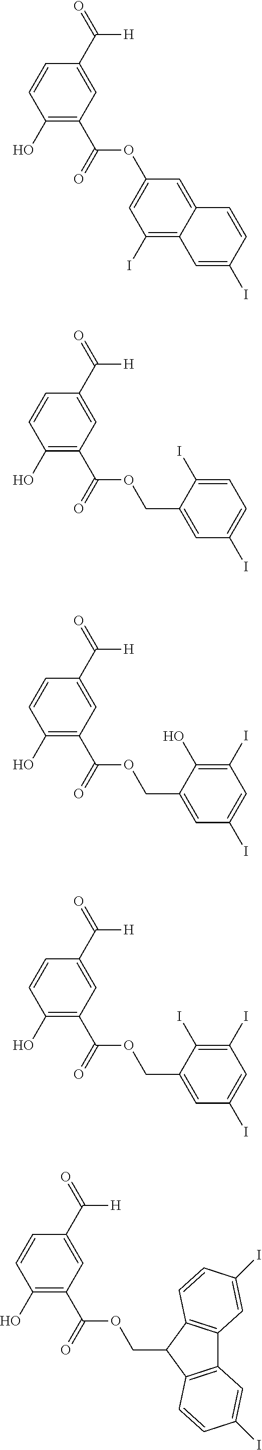 [SMILES: O=Cc1ccc(O)c(C(=O)Oc2cc(I)c3cc(I)ccc3c2)c1 ; O=Cc1ccc(O)c(C(=O)OCc2cc(I)ccc2I)c1 ; O=Cc1ccc(O)c(C(=O)OCc2cc(I)cc(I)c2O)c1 ; O=Cc1ccc(O)c(C(=O)OCc2cc(I)cc(I)c2I)c1 ; O=Cc1ccc(O)c(C(=O)OCC2c3ccc(I)cc3-c3cc(I)ccc32)c1]

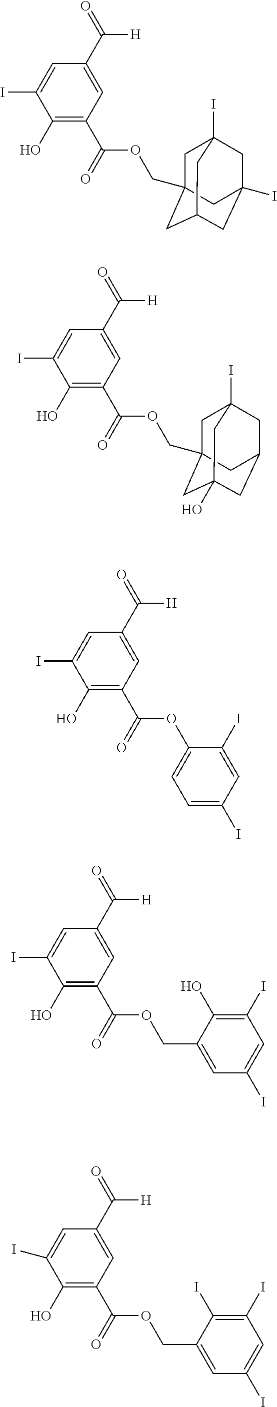 [SMILES: O=Cc1cc(I)c(O)c(C(=O)OCC23CC4CC(I)(CC(I)(C4)C2)C3)c1 ; O=Cc1cc(I)c(O)c(C(=O)OCC23CC4CC(O)(CC(I)(C4)C2)C3)c1 ; O=Cc1cc(I)c(O)c(C(=O)Oc2ccc(I)cc2I)c1 ; O=Cc1cc(I)c(O)c(C(=O)OCc2cc(I)cc(I)c2O)c1 ; O=Cc1cc(I)c(O)c(C(=O)OCc2cc(I)cc(I)c2I)c1]

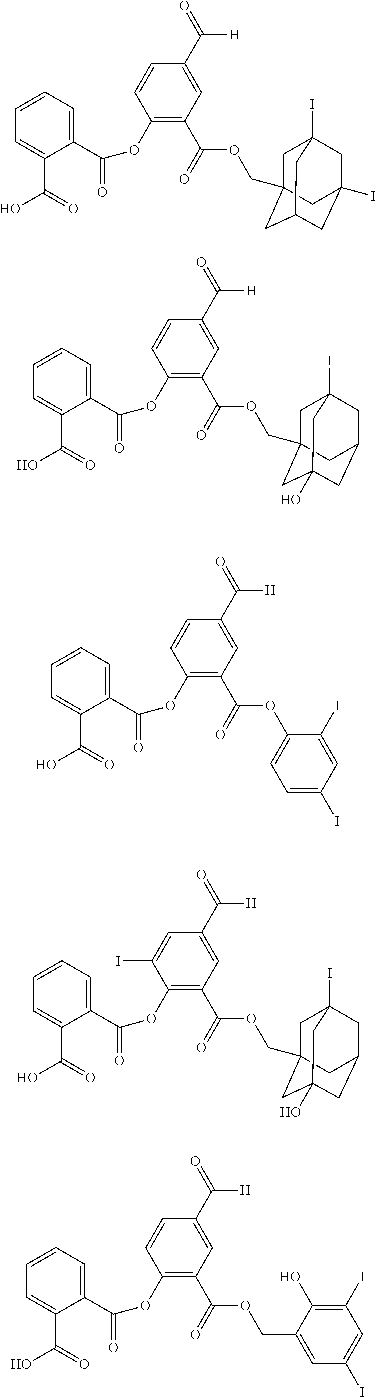 [SMILES: O=Cc1ccc(OC(=O)c2ccccc2C(=O)O)c(C(=O)OCC23CC4CC(I)(CC(I)(C4)C2)C3)c1 ; O=Cc1ccc(OC(=O)c2ccccc2C(=O)O)c(C(=O)OCC23CC4CC(O)(CC(I)(C4)C2)C3)c1 ; O=Cc1ccc(OC(=O)c2ccccc2C(=O)O)c(C(=O)Oc2ccc(I)cc2I)c1 ; O=Cc1cc(I)c(OC(=O)c2ccccc2C(=O)O)c(C(=O)OCC23CC4CC(O)(CC(I)(C4)C2)C3)c1 ; O=Cc1ccc(OC(=O)c2ccccc2C(=O)O)c(C(=O)OCc2cc(I)cc(I)c2O)c1]

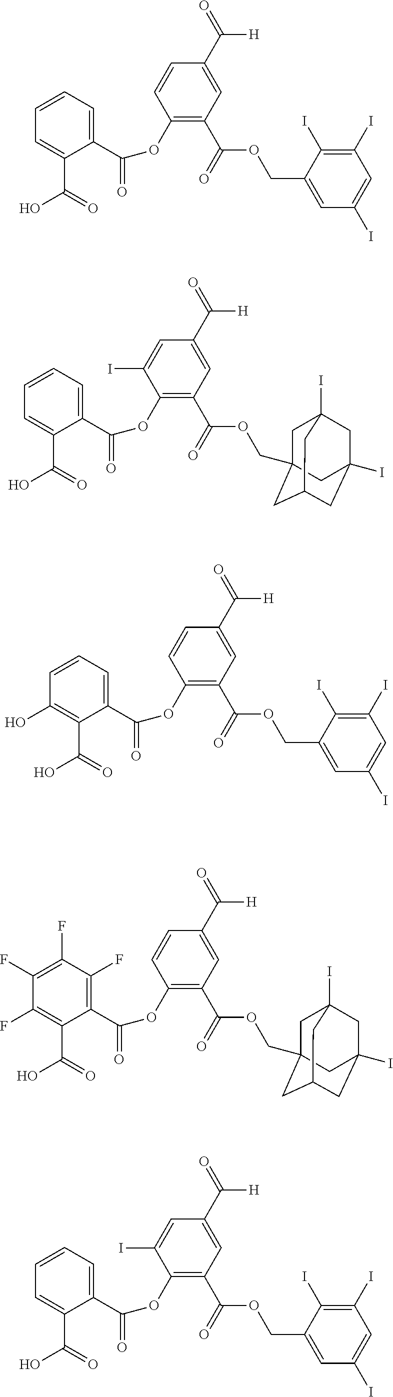 [SMILES: O=Cc1ccc(OC(=O)c2ccccc2C(=O)O)c(C(=O)OCc2cc(I)cc(I)c2I)c1 ; O=Cc1cc(I)c(OC(=O)c2ccccc2C(=O)O)c(C(=O)OCC23CC4CC(I)(CC(I)(C4)C2)C3)c1 ; O=Cc1ccc(OC(=O)c2cccc(O)c2C(=O)O)c(C(=O)OCc2cc(I)cc(I)c2I)c1 ; O=Cc1ccc(OC(=O)c2c(F)c(F)c(F)c(F)c2C(=O)O)c(C(=O)OCC23CC4CC(I)(CC(I)(C4)C2)C3)c1 ; O=Cc1cc(I)c(OC(=O)c2ccccc2C(=O)O)c(C(=O)OCc2cc(I)cc(I)c2I)c1]

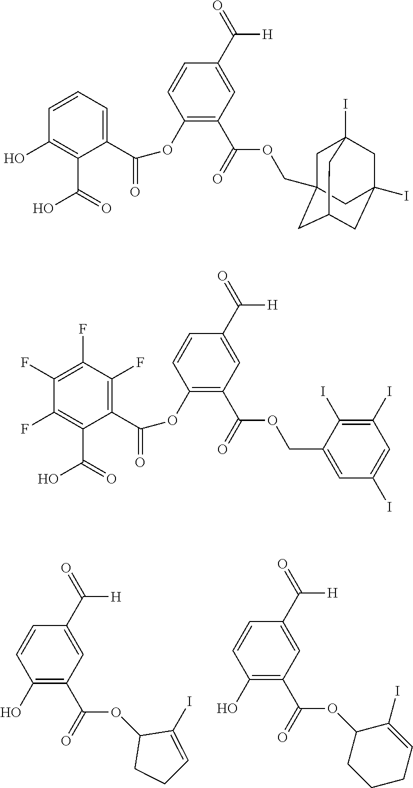

A compound in which X is a group represented by any of formulas (X1-1′), (X1-2′), (X1-4′), and (X1-5′) in the compound (I-C) (hereinafter, may be referred to as a compound (I-C)) can be obtained by reacting a compound represented by the formula (I1-a) with carbonyl diimidazole in a solvent, and then further reacting with a compound represented by a formula (I1-b) (acid treatment, as necessary).

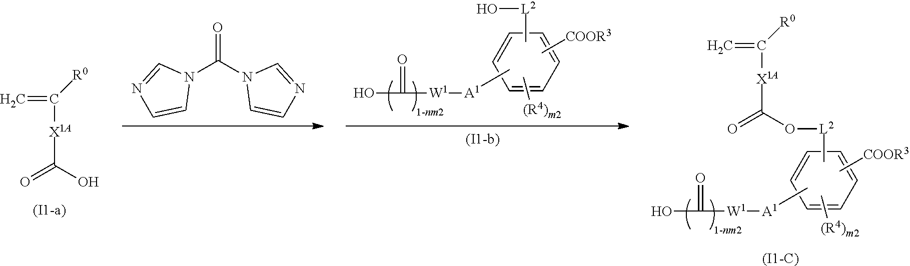

(In the formula, X^{1A} represents a single bond, —O—, —Ar¹—, or —CO—O— Ar¹⁻*, and Ar¹ represents a 1,4-phenylene group, * represents a binding site to the carbon atom of the carbonyl group. All reference numerals are the same as defined above)

Examples of the solvent include tetrahydrofuran, chloroform, and acetonitrile.

The reaction temperature is usually 0° C. to 80° C., and the reaction timc is usually 0.5 hour to 24 hours.

Examples of the compound represented by formula (I1-a) include compounds represented by the following formulas and the like and are easily available on the market, and can also be easily produced by a known production method.

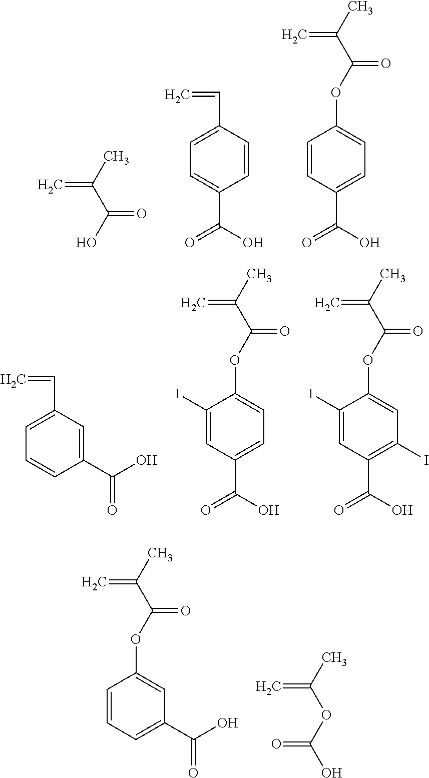

Examples of the compound represented by the formula (I1-b) include compounds represented by the following formulas and the like and are easily available on the market, and can also be easily produced by a known production method.

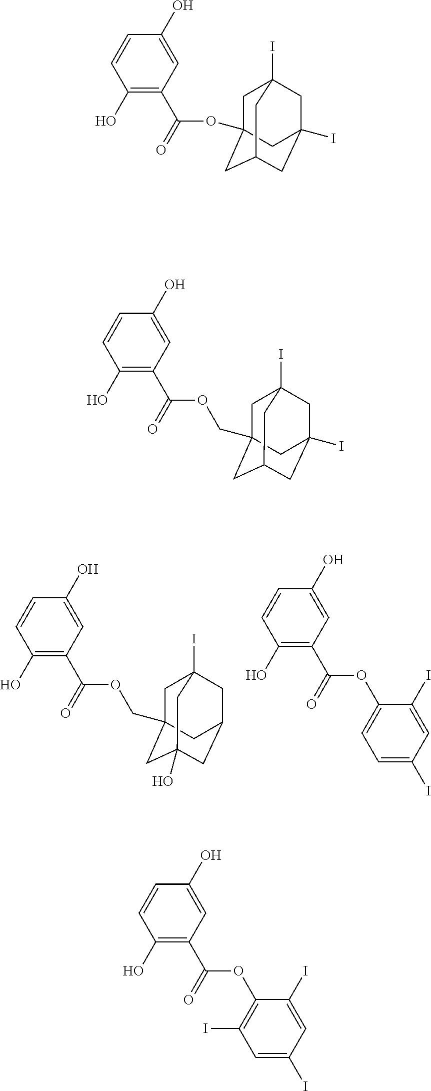

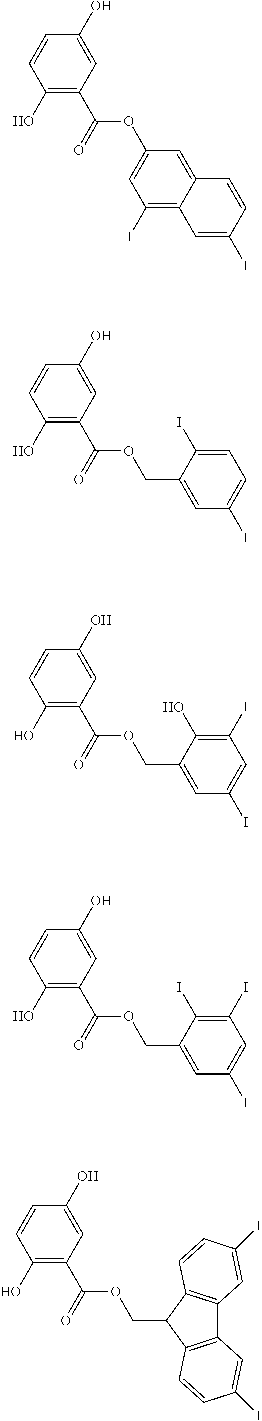 [SMILES: O=C(Oc1cc(I)c2cc(I)ccc2c1)c1cc(O)ccc1O ; O=C(OCc1cc(I)ccc1I)c1cc(O)ccc1O ; O=C(OCc1cc(I)cc(I)c1O)c1cc(O)ccc1O ; O=C(OCc1cc(I)cc(I)c1I)c1cc(O)ccc1O ; O=C(OCC1c2ccc(I)cc2-c2cc(I)ccc21)c1cc(O)ccc1O]

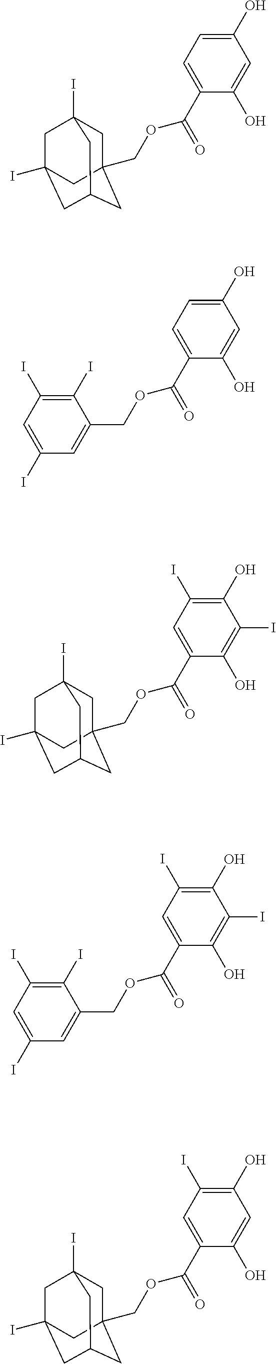 [SMILES: O=C(OCC12CC3CC(I)(CC(I)(C3)C1)C2)c1ccc(O)cc1O ; O=C(OCc1cc(I)cc(I)c1I)c1ccc(O)cc1O ; O=C(OCC12CC3CC(I)(CC(I)(C3)C1)C2)c1cc(I)c(O)c(I)c1O ; O=C(OCc1cc(I)cc(I)c1I)c1cc(I)c(O)c(I)c1O ; O=C(OCC12CC3CC(I)(CC(I)(C3)C1)C2)c1cc(I)c(O)cc1O]

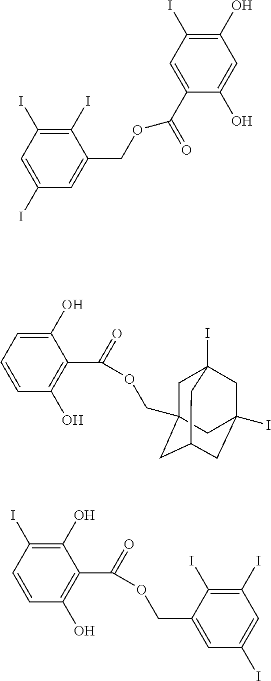 [SMILES: O=C(OCc1cc(I)cc(I)c1I)c1cc(I)c(O)cc1O ; O=C(OCC12CC3CC(I)(CC(I)(C3)C1)C2)c1c(O)cccc1O ; O=C(OCc1cc(I)cc(I)c1I)c1c(O)ccc(I)c1O]

A compound in which X¹ is a group represented by —O—, a formula (X-3′), formula (X-6′), or the like in the compound (I-C) (compound represented by a formula (I2-C)) can be obtained by reacting a compound represented by a formula (I2-a) and a compound represented by a formula (I2-b) in a solvent in the presence of a base (acid treatment, as necessary).

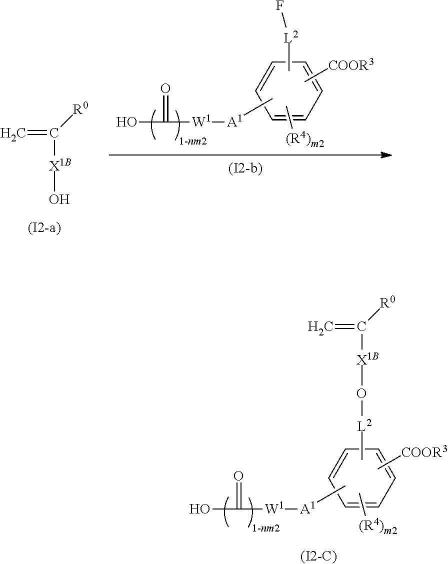

(In the formula, X^{1B} represents a single bond, —Ar¹—, or —CO—O—Ar¹—*. * represents a binding site to the oxygen atom. All reference numerals are the same as defined above)

Examples of the solvent include tetrahydrofuran, chloroform, and acetonitrile.

Examples of the base include potassium hydroxide.

The reaction temperature is usually 0° C. to 80° C., and the reaction timc is usually 0.5 hour to 24 hours.

Examples of the compound represented by a formula (12-a) include compounds represented by the following formulas and the like and are easily available on the market, and can also be easily produced by a known production method.

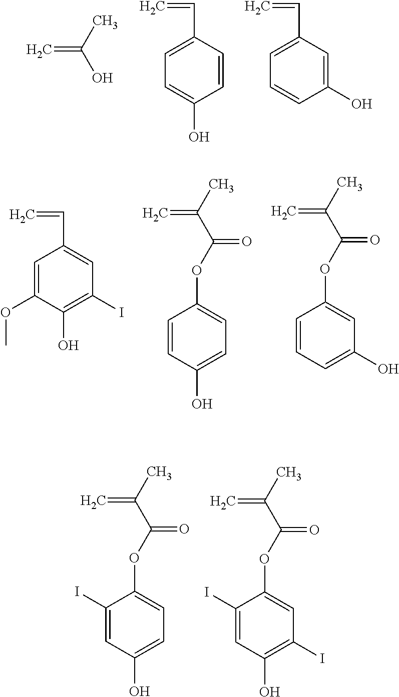

Examples of the compound represented by formula (I2-b) include compounds represented by the following formulas and the like and are easily available on the market, and can also be easily produced by a known production method.

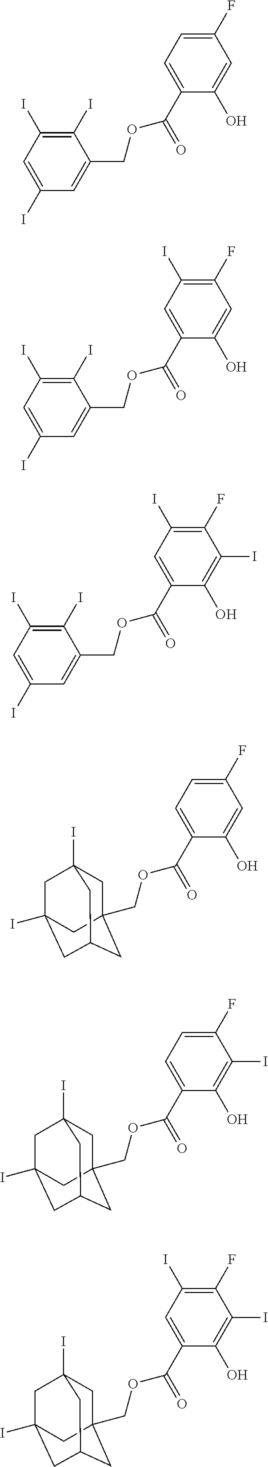 [SMILES: O=C(OCc1cc(I)cc(I)c1I)c1ccc(F)cc1O ; O=C(OCc1cc(I)cc(I)c1I)c1cc(I)c(F)cc1O ; O=C(OCc1cc(I)cc(I)c1I)c1cc(I)c(F)c(I)c1O ; O=C(OCC12CC3CC(I)(CC(I)(C3)C1)C2)c1ccc(F)cc1O ; O=C(OCC12CC3CC(I)(CC(I)(C3)C1)C2)c1ccc(F)c(I)c1O ; O=C(OCC12CC3CC(I)(CC(I)(C3)C1)C2)c1cc(I)c(F)c(I)c1O]

A compound in which X¹ is a group represented by a formula (X¹-7′), a formula (X¹-8′), or the like in the compound (I-C) (the compound represented by a formula (I3-C)) can be obtained by reacting a compound represented by the formula (I2-a) with a carbonyl diimidazole in a solvent, and then further reacting with a compound represented by the formula (I1-b) (acid treatment, as necessary).

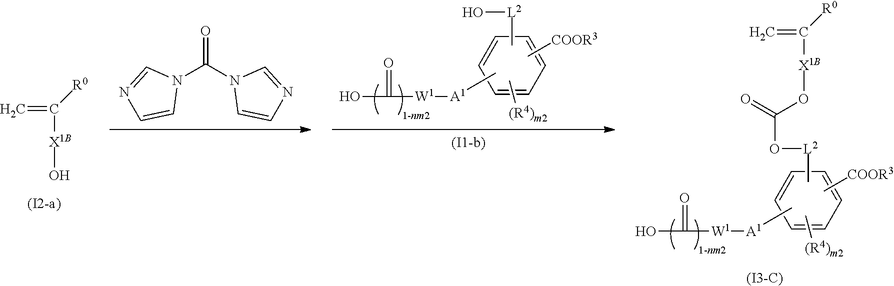

(In the formula, X^{1B} represents —Ar¹— or —CO—O—Ar¹⁻*. * represents a binding site to the oxygen atom. All reference numerals are the same as defined above)

Examples of the solvent include tetrahydrofuran, chloroform, and acetonitrile.

The reaction temperature is usually 0° C. to 80° C., and the reaction timc is usually 0.5 hour to 24 hours.

It is possible to produce a compound being a group in which L² is a group obtained by combining an alkanediyl group having 1 to 4 carbon atoms with an alicyclic hydrocarbon group having 3 to 18 carbon atoms in the compound (I-C) (in which —CH₂— included in the alkanediyl group may be substituted with —O— or —CO—, and —CH₂— included in the alicyclic hydrocarbon group may be substituted with —O—, —S—, —SO₂— or —CO—), for example, by replacing a compound represented by a formula (I1-a) or a compound represented by a formula (12-a) by compounds represented by the following formulas when synthesizing compounds represented by a formula (I1-C) to formula (I3-C).

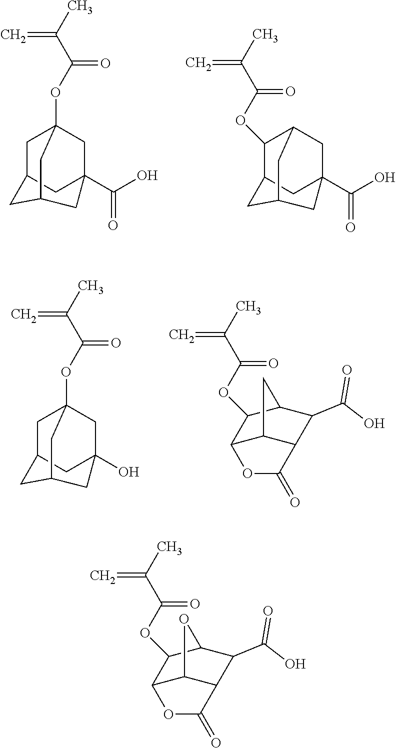 [SMILES: C=C(C)C(=O)OC12CC3CC(C1)CC(C(=O)O)(C3)C2 ; C=C(C)C(=O)OC1C2CC3CC1CC(C(=O)O)(C3)C2 ; C=C(C)C(=O)OC12CC3CC(CC(O)(C3)C1)C2 ; C=C(C)C(=O)OC1C2CC3C1OC(=O)C3C2C(=O)O ; C=C(C)C(=O)OC1C2OC(=O)C3C2OC1C3C(=O)O]

[Structural Unit Derived from Salt Represented by Formula (I)]

The structural unit derived from a salt represented by the formula (I) of the present invention is a structural unit represented by the following formula (IP) (hereinafter, may be referred to as “structural unit (IP)”).

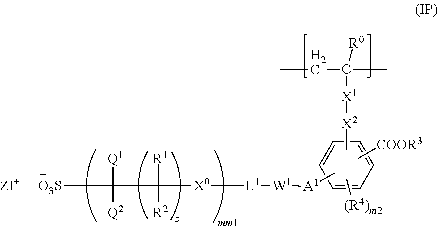

[In the formula (IP), all reference numerals are the same as defined above]

The structural unit (IP) indicates a state where a double bond of CH₂═C—R⁰ included in the salt (I) is cleaved.

Such structural unit (IP) functions as an acid generator similarly to the salt (I), and also functions as a structural unit constituting a compound or a resin.

[Resin Including Structural Unit (IP) Derived from Salt Represented by Formula (I)]

The resin of the present invention is a resin including a structural unit (IP) (hereinafter may be referred to as “resin (Ap)”).

The resin (Ap) may be either a homopolymer including one structural unit (IP), or a copolymer including two or more structural units (IP).

The resin (Ap) may include a structural unit other than the structural unit (IP). As described below, examples of the structural unit other than the structural unit (IP) include a structural unit having an acid-labile group (hereinafter may be referred to as “structural unit (a1)”), and a structural unit other than the structural unit (a1). Examples of the structural unit other than the structural unit (a1) include a structural unit having no acid-labile group (hereinafter may be referred to as “structural unit (s)”), and structural units known in the relevant field. Herein, “acid-labile group” means a group having a leaving group that is eliminated by contact with an acid, thus converting a constitutional unit into a constitutional unit having a hydrophilic group (for example, a hydroxy group or a carboxy group).

The content of the structural unit (IP) is typically 0.1 mol % or more, preferably 0.5 mol % or more, more preferably 0.8 mol % or more, and still more preferably 1 mol % or more, based on all structural units of the resin (Ap). The content is also usually 100 mol % or less, preferably 50 mol % or less, more preferably 30 mol % or less, and still more preferably 20 mol % or less. Specifically, the content is usually 0.1 to 100 mol %, preferably 0.5 to 50 mol %, more preferably 0.8 to 30 mol %, and still more preferably 1 to 20 mol %.

Particularly, when used for the resist composition, as described below, the resin (Ap) preferably includes, in addition to the structural unit (IP), a structural unit (a1).

When used for the resist composition, as described below, the resin (Ap) may be used in combination with a resin including a structural unit (a1) (hereinafter sometimes referred to as “resin (A)”) and/or a resin or the like other than the resin (A), whether the resin includes the structural unit (a1) or not. Hereinafter the resin (Ap) and/or the resin (A) may be referred to as “resin (A) or the like”.

It is preferable that each of the resin (Ap) and the resin (A) further includes a structural unit other than the structural unit (a1).

<Structural Unit (a1)>

The structural unit (a1) is derived from a monomer having an acid-labile group (hereinafter may be referred to as “monomer (a1)”).

The acid-labile group included in the resin (A) or the like is preferably a group represented by the formula (1) (hereinafter also referred to as group (1)) and/or a group represented by the formula (2) (hereinafter also referred to as group (2)):

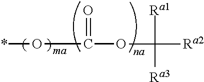

[In the formula (1), R^{a1}, R^{a2}, and R^{a3} each independently represent an alkyl group having 1 to 8 carbon atoms, an alkenyl group having 2 to 8 carbon atoms, an alicycic hydrocarbon group having 3 to 20 carbon atoms, an aromatic hydrocarbon group having 6 to 18 carbon atoms, or a group obtained by combining these groups, or R^{a1} and R^{a2} are bonded to each other to form an alicyclic hydrocarbon group having 3 to 20 carbon atoms together with the carbon atom to which they are bonded, R^{a3} represents an alkyl group having 1 to 8 carbon atoms, an alkenyl group having 2 to 8 carbon atoms, an alicyclic hydrocarbon group having 3 to 20 carbon atoms, an aromatic hydrocarbon group having 6 to 18 carbon atoms, or a group obtained by combining these groups, and the alkyl group, the alkenyl group, the alicyclic hydrocarbon group, and the aromatic hydrocarbon group may have a halogen atom.

ma and na each independently represent 0 or 1, and at least one of ma and na represents 1.

* represents a binding site.

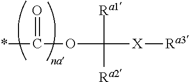

[In the formula (2), R^{a1′} and R^{a2′} each independently represent a hydrogen atom or a hydrocarbon group having 1 to 12 carbon atoms, R^{a3′} represents a hydrocarbon group having 1 to 20 carbon atoms or R^{a1′} represents a hydrogen atom or a hydrocarbon group having 1 to 12 carbon atoms, R^{a2′} and R^{a3′} are bonded to each other to form a heterocyclic ring group having 3 to 20 carbon atoms together with carbon atoms and X to which R^{a2′} and R^{a3′} are bonded, —CH₂— included in the hydrocarbon group and the heterocyclic ring group may be substituted with —O— or —S—, and the hydrocarbon group and the heterocyclic ring group may have a halogen atom.

X represents an oxygen atom or a sulfur atom.

na′ represents 0 or 1.

* represents a binding site.

Examples of the alkyl group in R^{a1}, R^{a2}, and R^{a3} include a methyl group, an ethyl group, a propyl group, a butyl group, a pentyl group, a hexyl group, a heptyl group, and an octyl group.

Examples of the alkenyl group in R^{a1}, R^{a2} and R^{a3} include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group, and a nonenyl group.

The alicyclic hydrocarbon group in R^{a1}, R^{a2} and R^{a3} may be either monocyclic or polycyclic. Examples of the monocyclic alicyclic hydrocarbon group include cycloalkyl groups such as a cyclopentyl group, a cyclohexyl group, a cycloheptyl group, and a cyclooctyl group. Examples of the polycyclic alicyclic hydrocarbon group include a decahydronaphthyl group, an adamantyl group, a norbornyl group, and the following groups (* represents a binding site). The number of carbon atoms of the alicyclic hydrocarbon group of R^{a1}, R^{a2}, and R^{a3} is preferably 3 to 16.

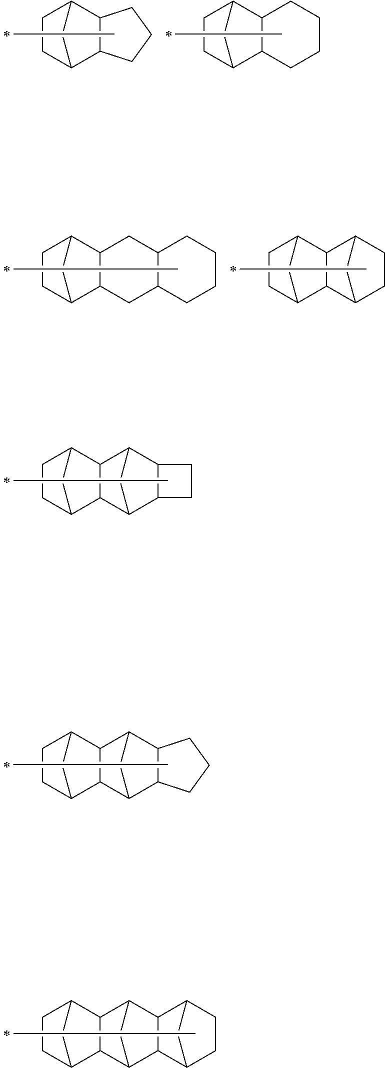

Examples of the aromatic hydrocarbon group in Rai, R^{a2}, and Ras include aryl groups such as a phenyl group, a naphthyl group, an anthryl group, a biphenyl group, and a phenanthryl group.

Examples of the combined group include groups obtained by combining the above-described alkyl group and alicyclic hydrocarbon group (for example, alkylcycloalkyl groups or cycloalkylalkyl groups, such as a methylcyclohexyl group, a dimethylcyclohexyl group, a methylnorbornyl group, a cyclohexylmethyl group, an adamantylmethyl group, an adamantyldimethyl group and a norbornylethyl group), aralkyl groups such as a benzyl group, aromatic hydrocarbon groups having an alkyl group (a p-methylphenyl group, a p-tert-butylphenyl group, a tolyl group, a xylyl group, a cumenyl group, a mesityl group, a 2,6-diethylphenyl group, a 2-methyl-6-ethylphenyl group, and the like), aromatic hydrocarbon groups having an alicyclic hydrocarbon group (a p-cyclohexylphenyl group, a p-adamantylphenyl group, and the like), and aryl-cycloalkyl groups such as a phenylcyclohexyl group.

Preferably, ma is 0 and na is 1.

When R^{a1} and R^{a2} are bonded to each other to form an alicyclic hydrocarbon group, examples of —C(R^{a1})(R^{a2})(R^{a3}) include the following groups. The number of carbon atoms of the alicyclic hydrocarbon group is preferably 3 to 12. * represents a binding site to —O—.

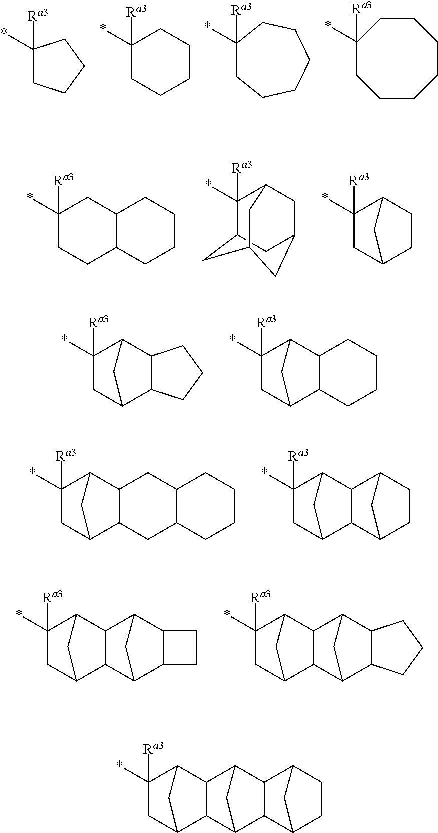

Examples of the hydrocarbon group in R^{a1′}, R^{a2′} and R^{a3′} include an alkyl group, an alicyclic hydrocarbon group, an aromatic hydrocarbon group, and groups formed by combining these groups.

Examples of the alkyl group, the alicyclic hydrocarbon group, the aromatic hydrocarbon group, and the groups obtained by combining these groups include those that are the same as described as for R^{a1}, R^{a2}, and R^{a3}.

When R^{a2′} and R^{a3′} are bonded to each other to form a heterocyclic group together with carbon atoms and X to which R^{a2′} and R^{a3′} are bonded, examples of —C(R^{a1′})(R^{a2′})—X—R^{a3′} include the following groups. * represents a binding site.

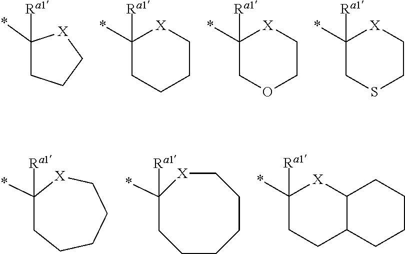

At least one of R^{a1′} and R^{a2′} is preferably a hydrogen atom.

na′ is preferably 0.

Examples of the halogen atom that may be possessed by R^{a1}, R^{a2}, R^{a3}, R^{a1}, R^{a2′}, and R^{a3′} include a fluorine atom, a chlorine atom, a bromine atom, or an iodine atom.

Examples of the group (1) include the following groups.

A group wherein, in the formula (1), R^{a1}, R^{a2}, and R^{a3} are alkyl groups, ma=0 and na=1. The group is preferably a tert-butoxycarbonyl group.

A group wherein, in the formula (1), R^{a1} and R^{a2} are bonded to each other to form an adamantyl group together with carbon atoms to which R^{a1} and R^{a2} are bonded, R^{a3} is an alkyl group, ma=0, and na=1.

A group wherein, in the formula (1), R^{a1} and R^{a2} are each independently an alkyl group, R^{a3} is an adamantyl group, ma=0, and na=1.

Specific examples of the group (1) include the following groups. * represents a binding site.

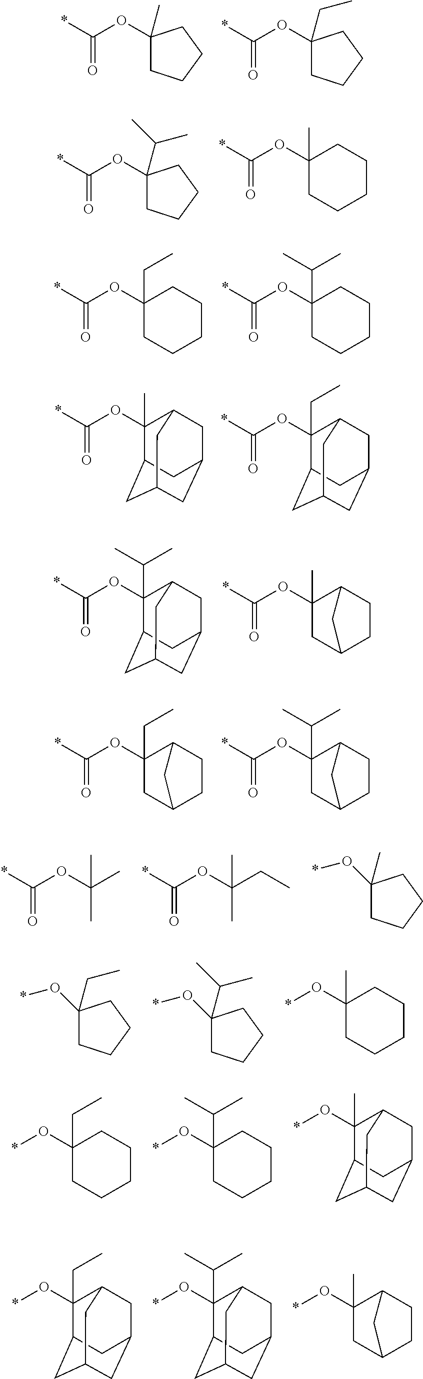

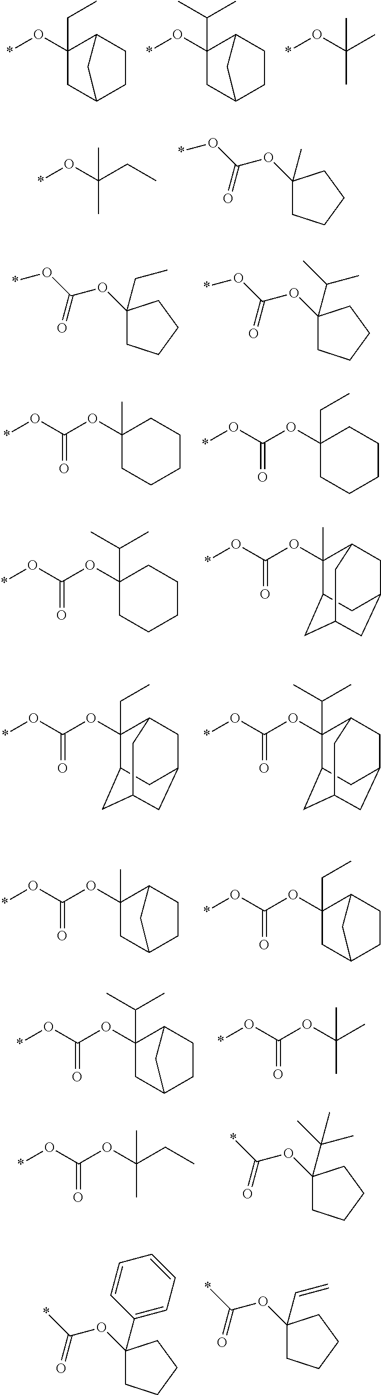

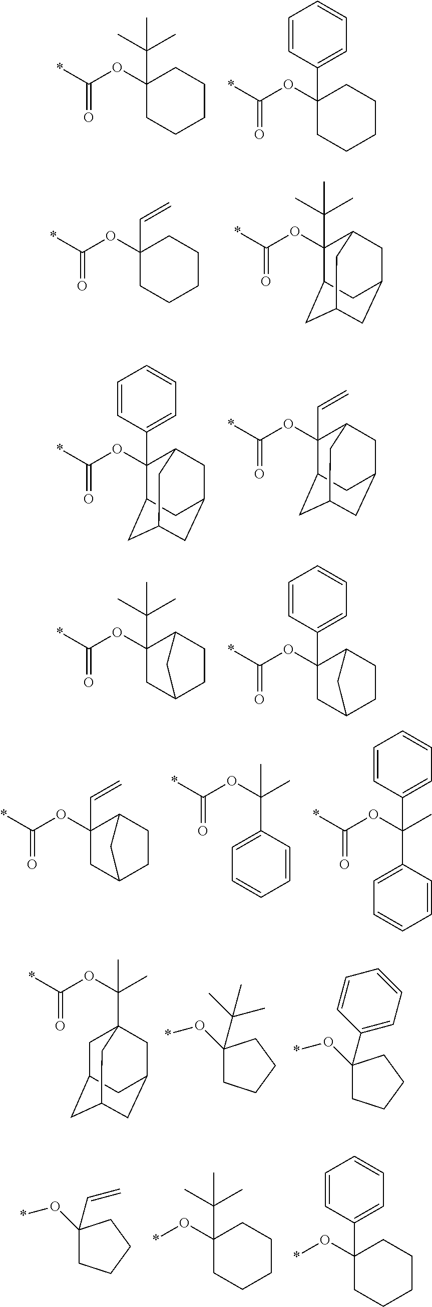

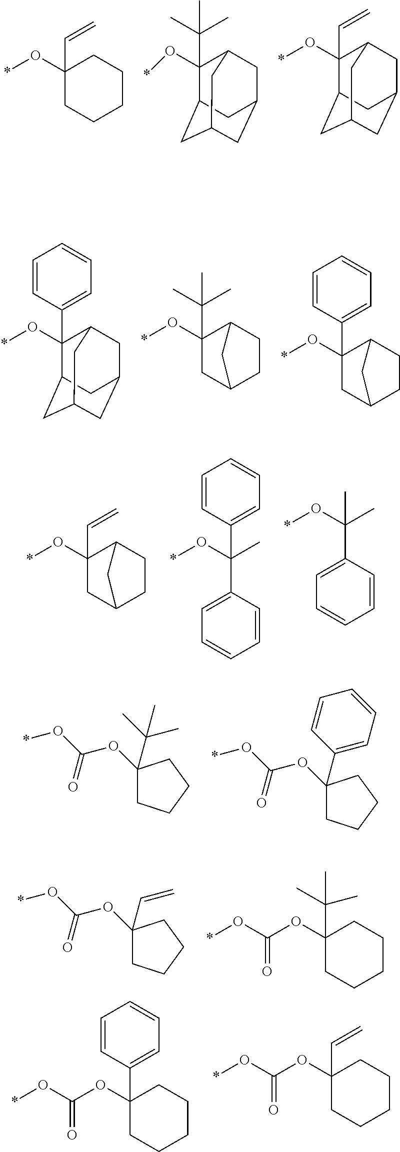

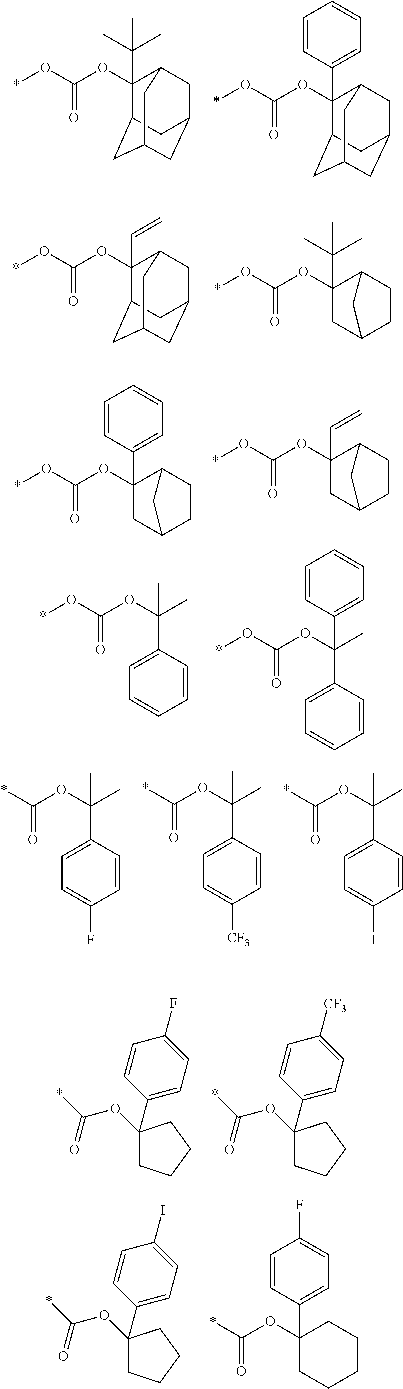

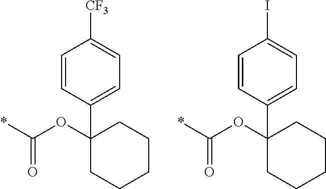

Specific examples of the group (2) include the following groups. * represents a binding site.

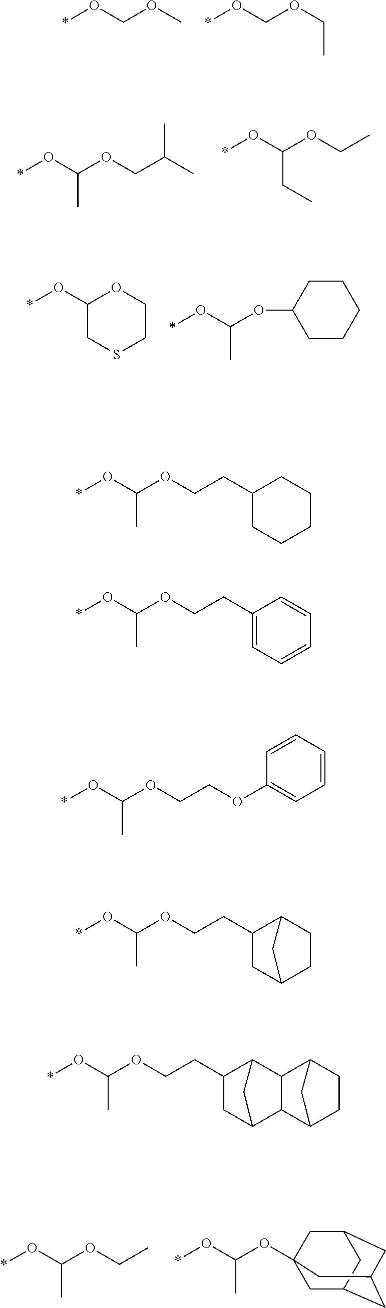

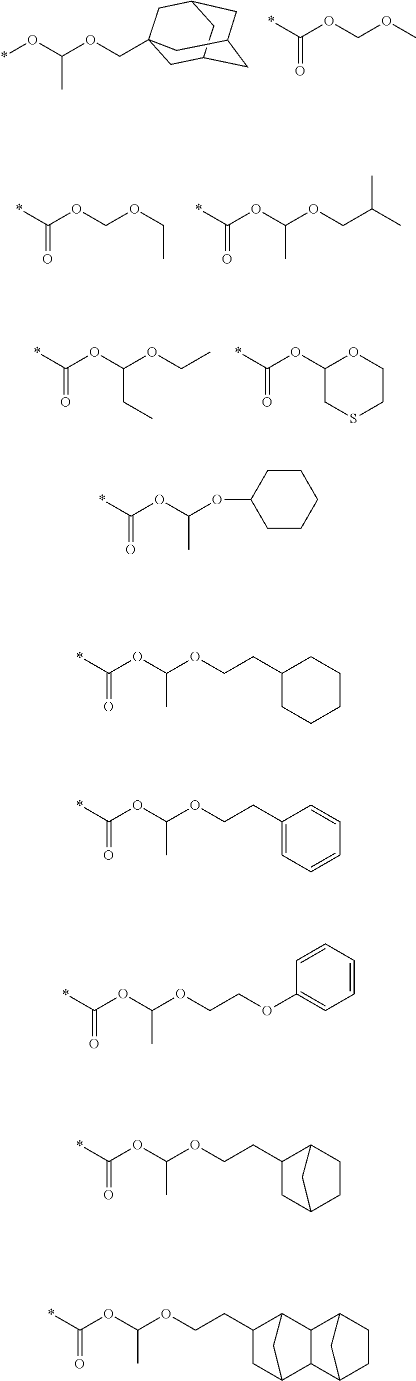

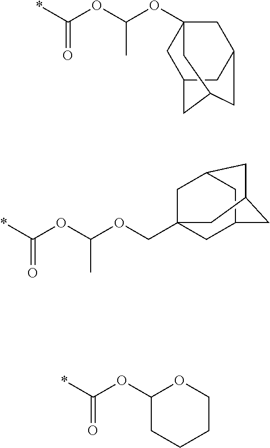

The monomer (a1) is preferably a monomer having an acid-labile group and an ethylenic unsaturated bond, and more preferably a (meth)acrylic monomer having an acid-labile group.

Among the (meth)acrylic monomers having an acid-labile group, those having an alicyclic hydrocarbon group having 5 to 20 carbon atoms are preferably exemplified. When using a resin (A) including a structural unit derived from a monomer (a1) having a bulky structure such as an alicyclic hydrocarbon group or the like in a resist composition, it is possible to improve the resolution of a resist pattern.

The structural unit derived from a (meth)acrylic monomer having a group (1) includes a structural unit represented by the formula (a1-0) (hereinafter may be referred to as structural unit (a1-0)), a structural unit represented by the formula (a1-1) (hereinafter may be referred to as structural unit (a1-1)) or a structural unit represented by the formula (a1-2) (hereinafter may be referred to as structural unit (a1-2)). The structural unit is preferably at least one structural unit selected from the group consisting of the structural unit (a1-0), the structural unit (a1-1), and the structural unit (a1-2), and more preferably at least one or two structural units selected from the group consisting of the structural unit (a1-1) and the structural unit (a1-2). These structural units may be used singly, or two or more structural units may be used in combination.

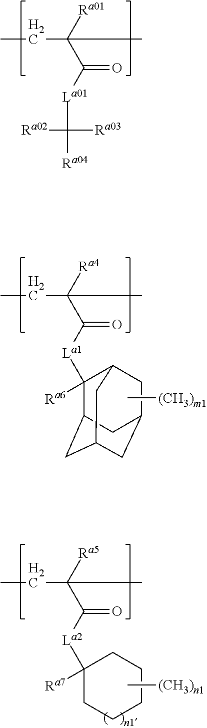

[In the formulas (a1-0), (a1-1), and (a1-2), all reference numerals have the same meaning as described above.]

Examples of the halogen atom in R^{a01}, R^{a4}, and R^{a5} include a fluorine atom, a chlorine atom, a bromine atom, or an iodine atom.

Examples of the alkyl group optionally having a halogen atom in R^{a01}, R^{a4}, and R^{a5} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a chloromethyl group, a bromomethyl group, and an iodomethyl group. The number of carbon atoms of the alkyl group is preferably 1 to 4, more preferably 1 to 3, and still more preferably 1 or 2. The alkyl group is preferably a methyl group or an ethyl group, and more preferably a methyl group.

R^{a01}, R^{a4}, and R^{a5} are preferably a hydrogen atom or a methyl group, and more preferably a methyl group.

L^{a01}, L^{a1}, and L^{a2} are preferably an oxygen atom or *—O—(CH₂)ₖ₀₁—CO— O— (in which k01 is preferably an integer of 1 to 4, and more preferably 1), and more preferably an oxygen atom.

Examples of the alkyl group, the alkenyl group, the alicyclic hydrocarbon group, the aromatic hydrocarbon group, and groups obtained by combining these groups in R^{a02}, R^{a03}, R^{a04}, R^{a6}, and R^{a7} include the same groups as described for R^{a1}, R^{a2}, and R^{a3} of the formula (1).

The alkyl group in R^{a02}, R^{a03}, and R^{a04} is preferably an alkyl group having 1 to 6 carbon atoms, more preferably a methyl group or an ethyl group, and still more preferably a methyl group.

The alkyl group in R^{a6} and R^{a7} is preferably an alkyl group having 1 to 6 carbon atoms, more preferably a methyl group, an ethyl group, an isopropyl group or a t-butyl group, and still more preferably an ethyl group, an isopropyl group, or a t-butyl group.

The alkenyl group in R^{a6} and R^{a7} is preferably an alkenyl group having 2 to 6 carbon atoms, and more preferably an ethenyl group, a propenyl group, an isopropenyl group, or a butenyl group.

The number of carbon atoms of the alicyclic hydrocarbon group as for R^{a02}, R^{a03}, R^{a04}, R^{a6}, and R^{a7} is preferably 5 to 12, and more preferably 5 to 10.

The number of carbon atoms of the aromatic hydrocarbon group of R^{a02}, R^{a03}, R^{a04}, R^{a6}, and R^{a7} is preferably 6 to 12, and more preferably 6 to 10.

The total number of carbon atoms of the groups obtained by combining the alkyl group with the alicyclic hydrocarbon group is preferably 18 or less.

The total number of carbon atoms of the groups obtained by combining the alkyl group with the aromatic hydrocarbon group is preferably 18 or less.

R^{a02} and R^{a03} are preferably an alkyl group having 1 to 6 carbon atoms and optionally having a halogen atom or an aromatic hydrocarbon group having 6 to 12 carbon atoms and optionally having a halogen atom, and more preferably a methyl group, an ethyl group, a phenyl group or a naphthyl group optionally having a halogen atom.

R^{a04} is preferably an alkyl group having 1 to 6 carbon atoms and optionally having a halogen atom or an alicyclic hydrocarbon group having 5 to 12 carbon atoms and optionally having a halogen atom, and more preferably a methyl group, an ethyl group, a cyclohexyl group, or an adamantyl group optionally having a halogen atom.

R^{a6} and R^{a7} are preferably an alkyl group having 1 to 6 carbon atoms and optionally having a halogen atom, an alkenyl group having 2 to 6 carbon atoms and optionally having a halogen atom, or an aromatic hydrocarbon group having 6 to 12 carbon atoms and optionally having a halogen atom, more preferably a methyl group, an ethyl group, an isopropyl group, a t-butyl group, an ethenyl group, a phenyl group, or a naphthyl group optionally having a halogen atom, and still more preferably an ethyl group, an isopropyl group, a t-butyl group, an ethenyl group, or a phenyl group optionally having a halogen atom.

m1 is preferably an integer of 0 to 3, and more preferably 0 or 1.

n1 is preferably an integer of any one of 0 to 10, more preferably an integer of any one of 0 to 8, still more preferably an integer of any one of 0 to 3, and still more preferably 0 or 1.

n1′ is preferably 0 or 1.

The structural unit (a1-0) includes, for example, a structural unit represented by any one of formula (a1-0-1) to formula (a1-0-24) and a structural unit in which a methyl group corresponding to R^{a01} in the structural unit (a1-0) is substituted with a hydrogen atom, a halogen atom, a haloalkyl group (an alkyl group having a halogen atom) or other alkyl groups, and is preferably a structural unit represented by any one of formula (a1-0-1) to formula (a1-0-10), formula (a1-0-13) and formula (a1-0-14).

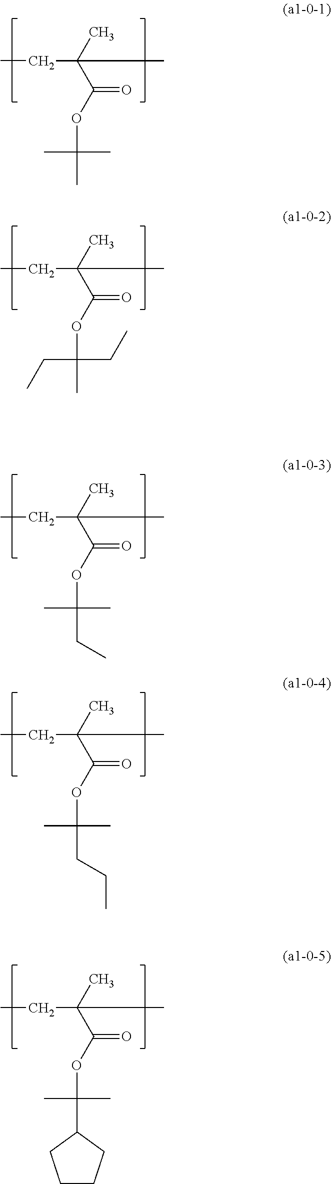

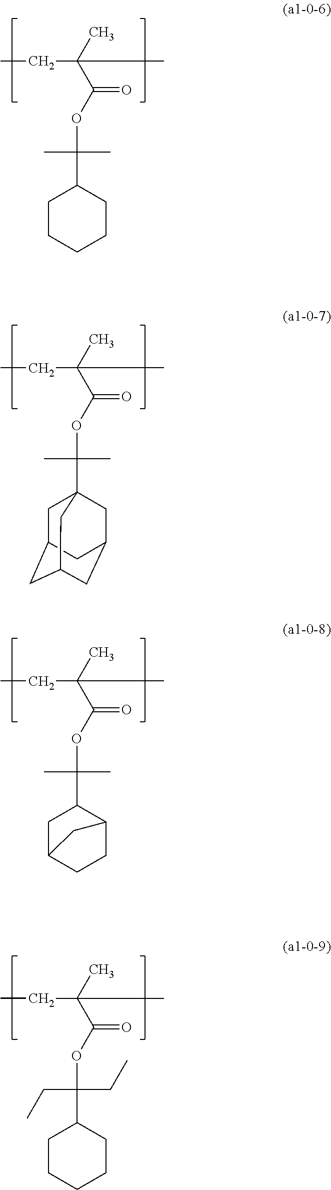

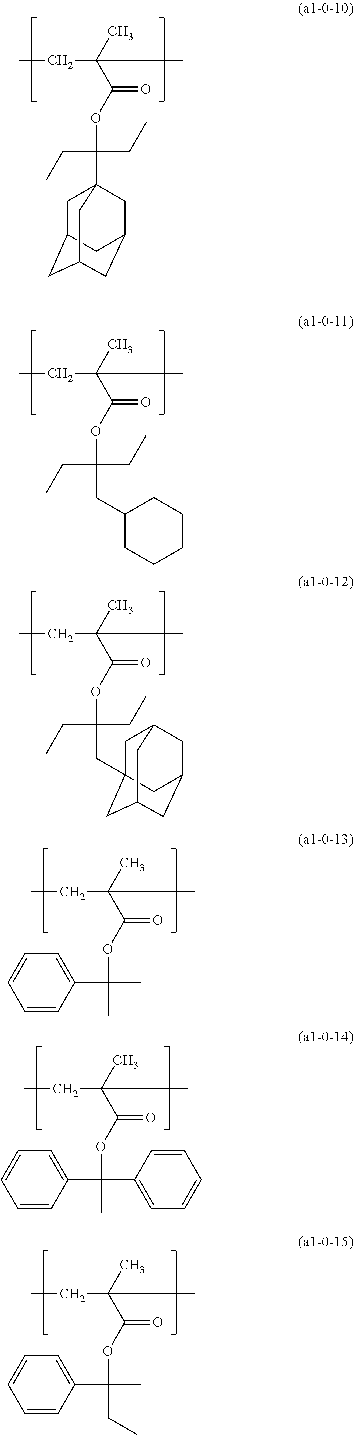

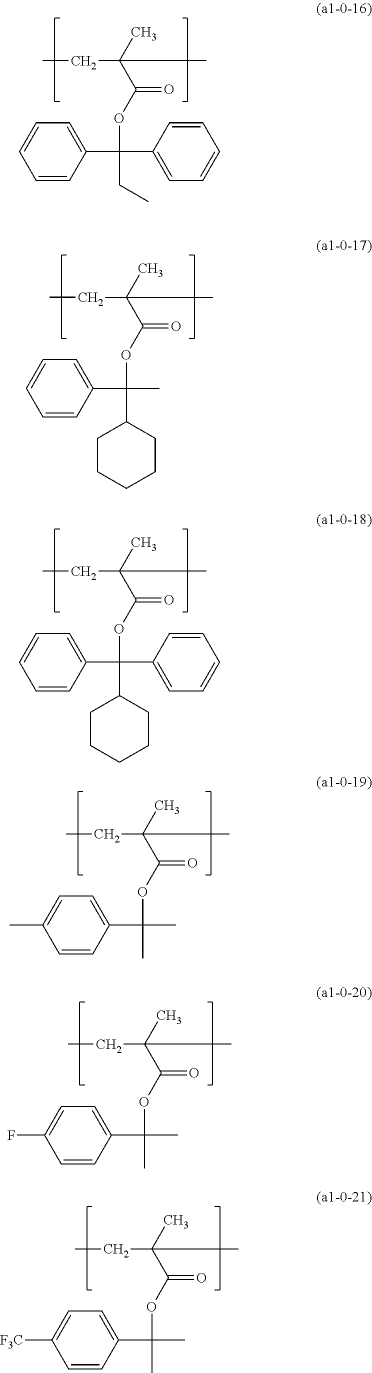

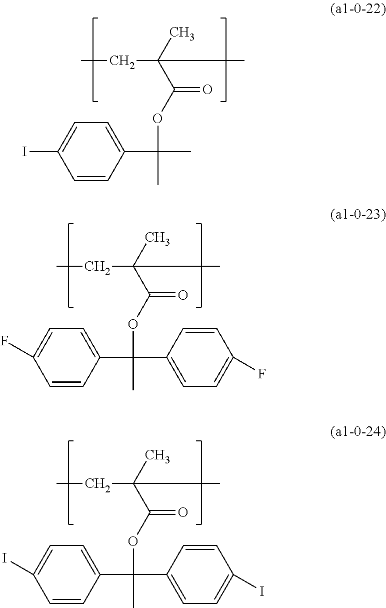

The structural unit (a1-1) includes, for example, structural units derived from the monomers described in JP-A-2010-204646. Among these structural units, a structural unit represented by any one of a formula (a1-1-1) to formula (a1-1-7) and a structural unit in which a methyl group corresponding to R^{a4} in the structural unit (a1-1) is substituted with a hydrogen atom, a halogen atom, a haloalkyl group, or other alkyl groups are preferable, and a structural unit represented by any one of the formula (a1-1-1) to formula (a1-1-4) is more preferable.

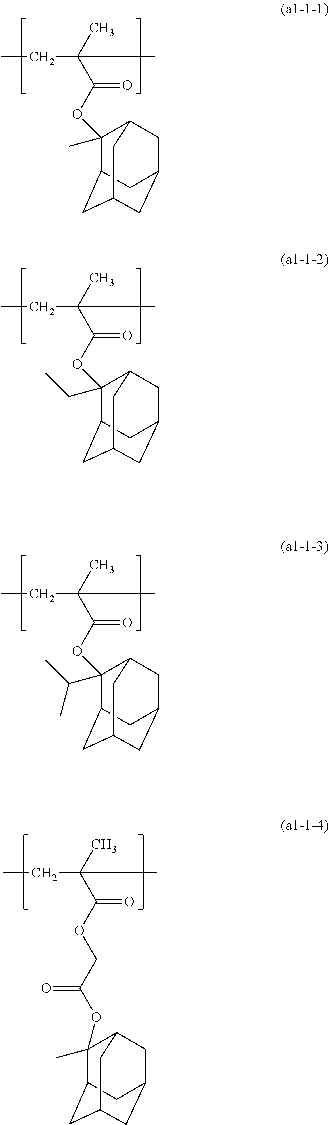

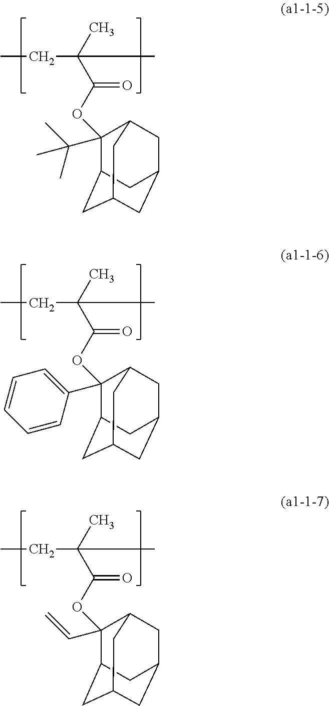

Examples of the structural unit (a1-2) include a structural unit represented by any one of formula (a1-2-1) to formula (a1-2-20), and a structural unit in which a methyl group corresponding to R^{a5} in the structural unit (a1-2) is substituted with a hydrogen atom, a halogen atom, a haloalkyl group or other alkyl groups, and a structural unit represented by any one of formula (a1-2-2), formula (a1-2-5), formula (a1-2-6) and formula (a1-2-10) to formula (a1-2-14) is preferable.

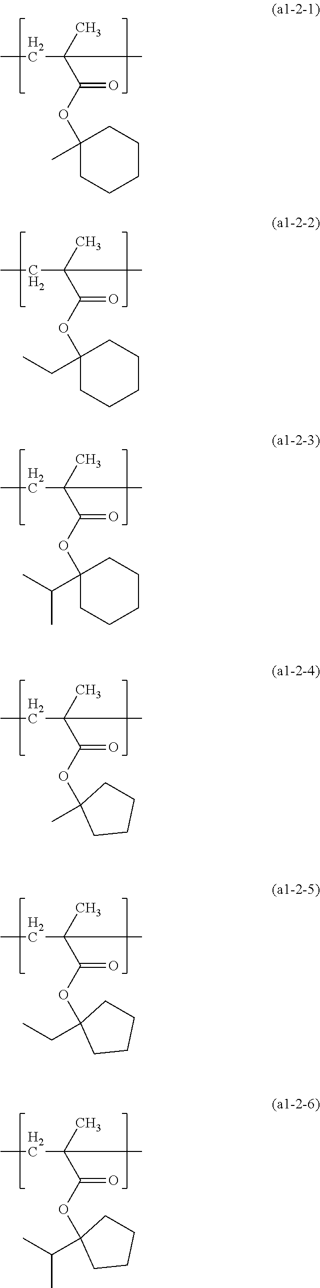

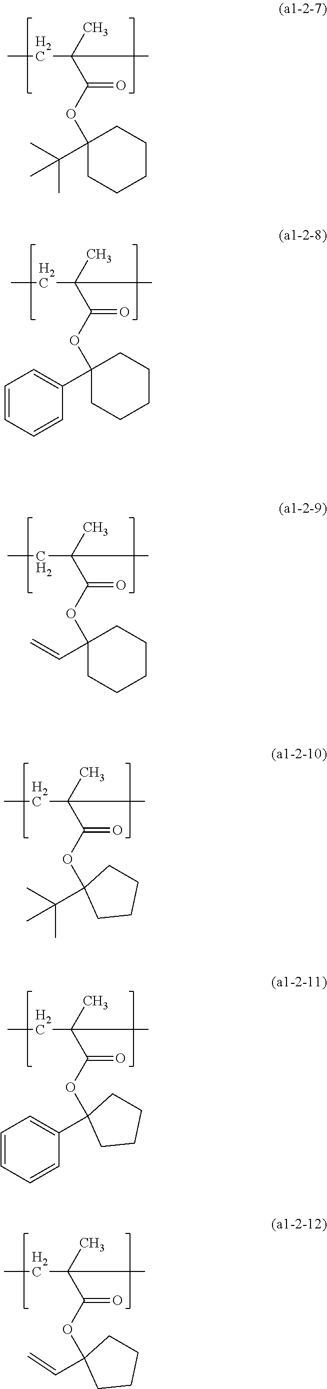

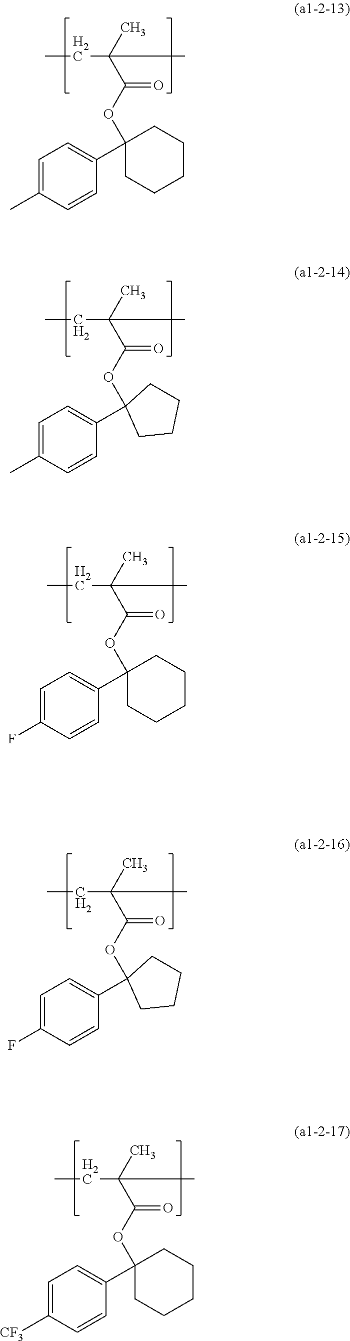

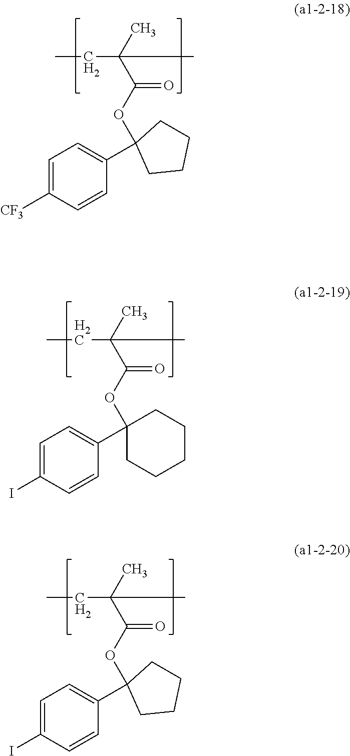

When the resin (A) or the like includes the structural unit (a1-0) and/or the structural unit (a1-1) and/or the structural unit (a1-2), the total content of them is typically 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. The total content is also typically 95 mol % or less, preferably 90 mol % or less, more preferably 85 mol % or less, still more preferably 70 mol % or less, and yet more preferably 65 mol % or less, based on all structural units of the resin (A) or the like. Specifically, the total content is typically 10 to 95 mol %, preferably 15 to 90 mol %, more preferably 20 to 85 mol %, still more preferably 25 to 70 mol %, and yet more preferably 30 to 70 mol %, based on all structural units of the resin (A) or the like.

When the resin (A) or the like includes the structural unit (a1-0), the content is typically 5 mol % or more, preferably 10 mol % or more, more preferably 15 mol % or more, still more preferably 20 mol % or more, yet more preferably 25 mol % or more, further preferably 30 mol % or more, and still further preferably 35 mol % or more, based on all structural units of the resin (A) or the like. The content is also typically 80 mol % or less, preferably 75 mol % or less, more preferably 70 mol % or less, still more preferably 65 mol % or less, and yet more preferably 60 mol % or less, based on all structural units of the resin (A) or the like. Specifically, the total content is typically 5 to 80 mol %, preferably 5 to 75 mol %, more preferably 10 to 70 mol %, still more preferably 10 to 65 mol %, and yet more preferably 20 to 60 mol %, based on all structural units of the resin (A) or the like.

When the resin (A) or the like includes the structural unit (a1-1) and/or the structural unit (a1-2), the total content of them is typically 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. The total content is also typically 95 mol % or less, preferably 90 mol % or less, more preferably 85 mol % or less, still more preferably 80 mol % or less, yet more preferably 75 mol % or less, further preferably 70 mol % or less, and still further preferably 65 mol % or less, based on all structural units of the resin (A) or the like. Specifically, the total content is typically 10 to 90 mol %, preferably 15 to 85 mol %, more preferably 15 to 80 mol %, still more preferably 20 to 80 mol %, yet more preferably 20 to 75 mol %, and further preferably 20 to 70 mol %, based on all structural units of the resin (A) or the like.

In the structural unit (a1), examples of the structural unit having the group (2) include a structural unit represented by a formula (a1-4) (hereinafter, may be referred to as “structural unit (a1-4)”).

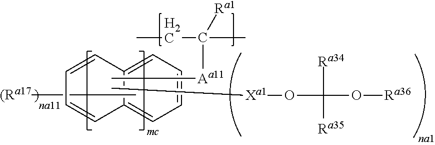

[In the formula (a1-4), all reference numerals are the same as defined above]

Examples of the halogen atom and the alkyl group optionally having a halogen atom in R^{a1} and R^{a17} include those that are the same as described as for R^{a01}, R^{a4}, and R^{a5} of the formula (a1-0), formula (a1-1), and formula (a1-2).

R^{a1} is preferably a hydrogen atom or an alkyl group having 1 to 4 carbon atoms, more preferably a hydrogen atom, a methyl group or an ethyl group, and still more preferably a hydrogen atom or a methyl group.

Examples of an alkoxy group, an alkoxyalkyl group, an alkoxyalkoxy group, an alkylcarbonyl group, and an alkylcarbonyloxy group in R^{a17} include the same groups as those exemplified in the present specification as long as the upper limit of the number of carbon atoms is acceptable. The number of carbon atoms in the alkoxy group is preferably 1 to 4, more preferably 1 to 3, still more preferably 1 to 2, and still more preferably 1. The number of carbon atoms in the alkoxyalkyl group is preferably 2 to 8, more preferably 2 to 4, still more preferably 1 to 2, and still more preferably 1. The number of carbon atoms in the alkoxyalkoxy group is preferably 2 to 8 and more preferably 2 to 4. The number of carbon atoms in the alkylcarbonyl group is preferably 2 to 4, more preferably 2 to 3, and still more preferably 2. The number of carbon atoms in the alkylcarbonyloxy group is preferably 2 to 4, more preferably 2 to 3, and still more preferably 2.

R^{a17} is preferably a halogen atom, a hydroxy group, a carboxy group, an alkyl group having 1 to 4 carbon atoms and optionally having a halogen atom, an alkoxy group having 1 to 4 carbon atoms, or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, and still more preferably a fluorine atom, an iodine atom, a hydroxy group, a methyl group, a methoxy group, or an ethoxyethoxy group.

Examples of the alkanediyl group in A^{a11} include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; and branched alkanediyl groups such as a butane-1,3-diyl group, a 2-methylpropane-1,3-diyl group, a 2-methylpropane-1,2-diyl group, a pentane-1,4-diyl group, a 2-methylbutane-1,4-diyl group, a heptane-1,6-diyl group, an octane-1,7-diyl group, a nonane-1,8-diyl group, a decane-1,9-diyl group and an undecane-1,10-diyl group. The number of carbon atoms of the alkanediyl group is preferably 1 to 10, more preferably 1 to 8, still more preferably 1 to 6, yet more preferably 1 to 4, further preferably 1 to 3, and still further preferably 1 or 2.

Examples of the alkyl group as for R^{a18} include a methyl group, an ethyl group, an n-propyl group, an isopropyl group, an n-butyl group, and a tert-butyl group.

When —CH₂— included in the alkanediyl group as for A^{a11} is substituted with —O—, —CO— or —NR^{a18}—, the number of carbon atoms before replacement is taken as the number of carbon atoms of the alkanediyl group.

Examples of the group in which —CH₂— in the alkanediyl group as for A^{a11} is substituted with —O—, —CO— or —NR^{a18}— include a hydroxy group, a carboxy group, a carbonyl group, an oxy group, an amino group, an alkoxy group, an alkoxycarbonyl group, an alkylcarbonyl group, an alkylamino group, a peptide group, an alkanediyloxy group, an alkanediyloxycarbonyl group, an alkanediylcarbonyl group, an alkanediylcarbonyloxy group, an alkanediylsulfonyl group, an alkanediylthio group, and an alkanediylamino group. Examples of these substituted groups include those that are the same as described herein as long as the upper limit of the number of carbon atoms is acceptable. The number of carbon atoms of these substituted groups is, for example, preferably 1 to 11, more preferably 1 to 6, still more preferably 1 to 4, and yet more preferably 1 to 3 in the case of an alkoxy group or the like, preferably 2 to 11, more preferably 2 to 6, still more preferably 2 to 4, and yet more preferably 2 or 3 in the case of an alkoxycarbonyl group or the like, preferably 2 to 12, more preferably 2 to 6, still more preferably 2 to 4, and yet more preferably 2 or 3 in the case of an alkylcarbonyl group or the like, and is preferably 2 to 10, more preferably 2 to 6, still more preferably 2 to 4, and yet more preferably 2 or 3 in the case of an alkoxycarbonyloxy group or the like.

Examples of the group in which —CH₂— included in the alkanediyl group as for A^{a11} is substituted with —O—, —CO— or —NR^{a18}— include, for example, *—O—, *—CO—O—, *—O—CO—, *—CO—O-A^{a12}-CO—O—, *—O—CO-A^{a12}-O—, *—O— A^{a12}-CO—O—, *—CO—O-A^{a12}-O—CO—, *—O—CO-A^{a12}-O—CO—, and *—CO—NR^{a18}-Among these, *—CO—O—, *—CO—O-A^{a12}-CO—O—, *—O-A^{a12}-CO—O—, or *—CO—NR^{a18}. is preferable. Herein, A^{a12} represents an alkanediyl group having 1 to 8 carbon atoms, * represents a binding site to carbon atoms to which R^{a1} is bonded. Examples of the alkanediyl group as for A^{a2} include the same alkanediyl group as for A^{a11} as long as the upper limit of the number of carbon atoms is acceptable.

A^{a11} is preferably a single bond, *—CO—O—, or *—CO—O-A^{a12}-CO—O—, more preferably a single bond, *—CO—O—, or *—CO—O—CH₂—CO—O—, and still more preferably a single bond or *—CO—O—.

na1 is preferably 1, 2, 3, or 4, more preferably 1, 2, or 3, and still more preferably 1 or 2.

na11 is preferably 0, 1, 2, or 3, more preferably 0, 1, or 2, and still more preferably 0 or 1. mc is preferably 0 or 1.

Examples of R^{a34}, R^{a35}, and R^{a36} include the same ones as those exemplified for R^{a1′}, R^{a2′}, and R^{a3′} in the formula (2).

When R^{a35} and R^{a36} are bonded to each other to form a divalent heterocyclic group having 3 to 20 carbon atoms together with —C—O— to which R^{a35} and R^{a36} are bonded, examples of the group include the following groups. * represents a binding site, and one of * is a binding site to R^{a34}.

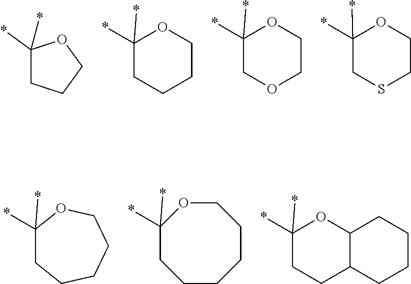

R^{a34} is preferably a hydrogen atom.

R^{a35} is preferably a hydrogen atom, an alkyl group having 1 to 12 carbon atoms or an alicyclic hydrocarbon group having 3 to 12 carbon atoms, and more preferably a methyl group or an ethyl group.

The hydrocarbon group of R^{a33} is preferably an alkyl group having 1 to 18 carbon atoms, an alicyclic hydrocarbon group having 3 to 18 carbon atoms, an aromatic hydrocarbon group having 6 to 18 carbon atoms, or groups formed by combining these groups, and more preferably an alkyl group having 1 to 18 carbon atoms, an alicyclic hydrocarbon group having 3 to 18 carbon atoms or an aralkyl group having 7 to 18 carbon atoms. The alkyl group and the alicyclic hydrocarbon group in R^{a36} are preferably unsubstituted. The aromatic hydrocarbon group in R^{a36} is preferably an aromatic ring having an aryloxy group having 6 to 10 carbon atoms.

—O—C(R^{a34})(R^{a35})—O—R^{a36} in the structural unit (a1-4) is eliminated by contacting with an acid (for example, p-toluenesulfonic acid) to form a hydroxy group or a carboxy group.

In the case of the benzene ring, —X^{a1}—O—C(R^{a34})(R^{a35})—O—R^{a36} may be bonded at the o-position, the in-position or the p-position, with respect to the binding site of A^{a11}. In particular, at least one is preferably bonded at the o-position or the p-position, and more preferably bonded at the p-position. In the case of the naphthalene ring, when the binding site of A^{a11} is the 1-position, it may be bonded at any of the 2-position to the 8-position, and when the binding site of A^{a11} is the 2-position, it may be bonded at any of the 1-position and the 3-position to the 8-position. In particular, when the binding site of A^{a11} is the 1-position, at least one is preferably bonded at the 3-position to the 6-position, and more preferably bonded at the 3-position or the 4-position. When the binding site of A^{a11} is the 2-position, it is preferably bonded at the 4-position to the 7-position, and more preferably bonded at the 5-position or the 6-position.

The structural unit (a1-4) includes, for example, structural units derived from the monomers described in JP-A-2010-204646. The structural unit preferably includes structural units represented by a formula (a1-4-1) to formula (a1-4-42) and a structural unit in which a hydrogen atom corresponding to R^{a1} in the structural unit (a1-4) is substituted with a halogen atom, a haloalkyl group, or an alkyl group, and more preferably structural units represented by the formula (a1-4-1) to formula (a1-4-5), formula (a1-4-10), formula (a1-4-13), formula (a1-4-14), formula (a1-4-19), and formula (a1-4-20).

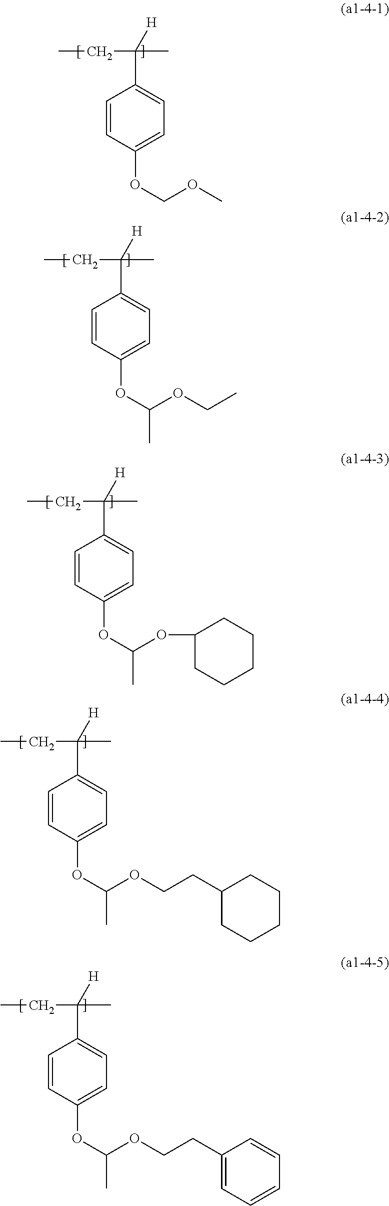

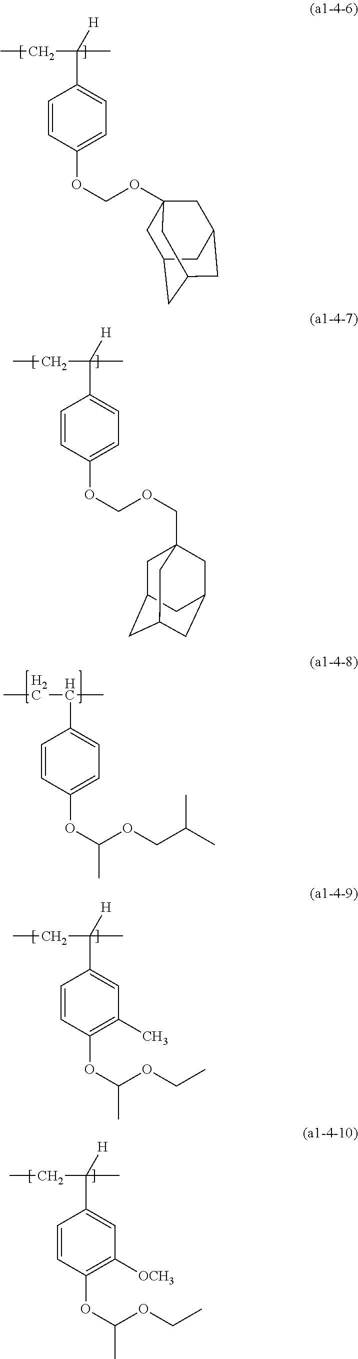

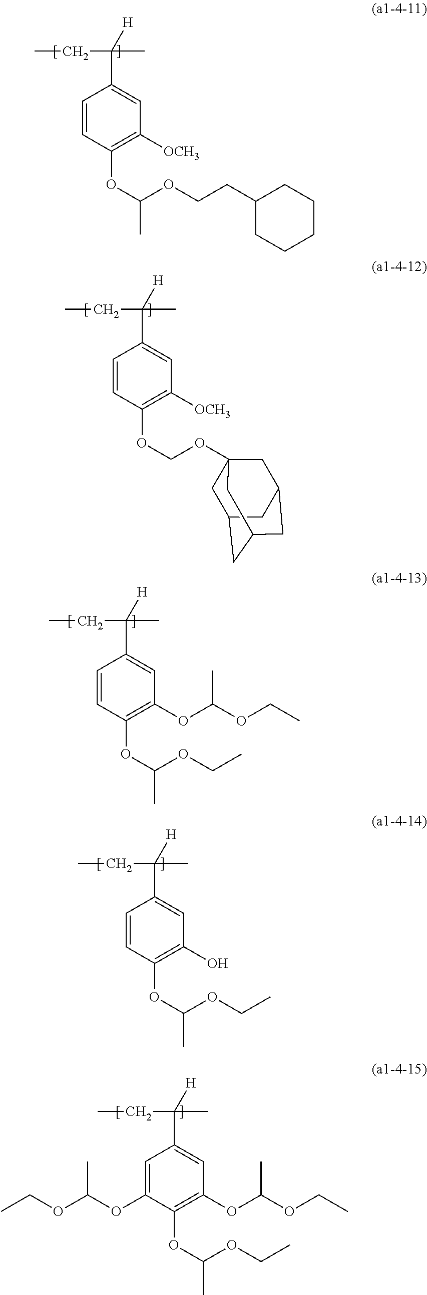

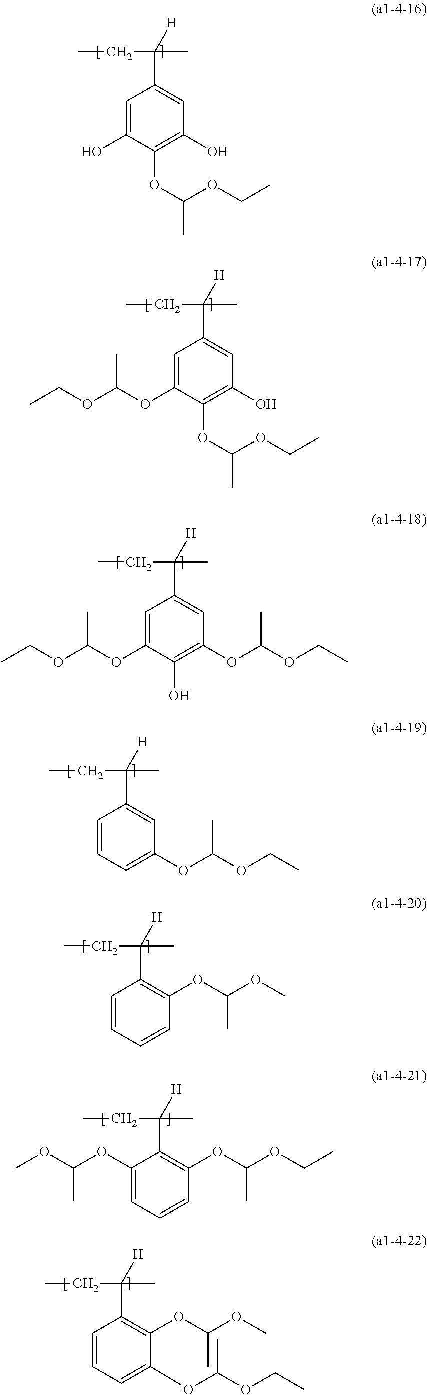

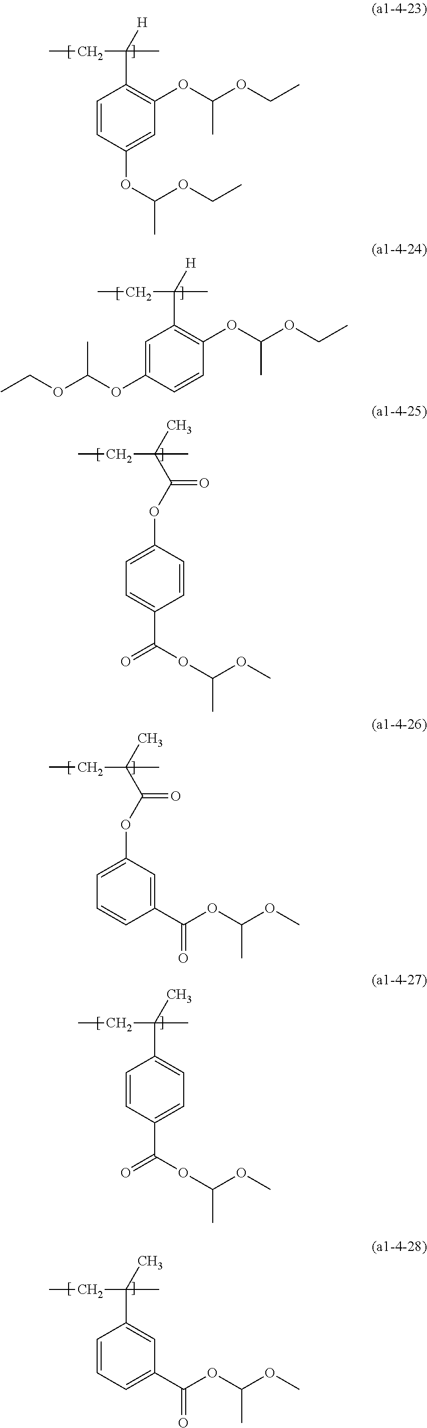

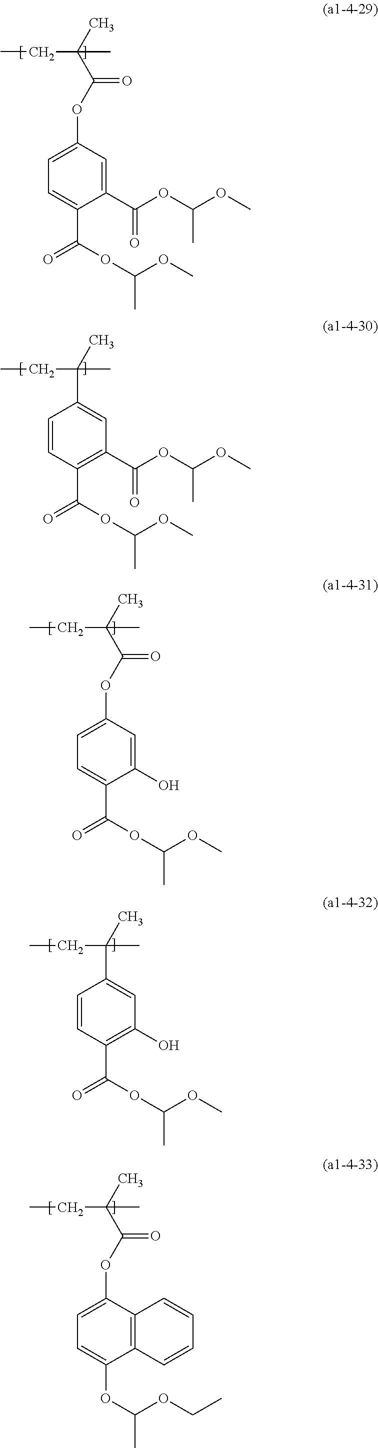

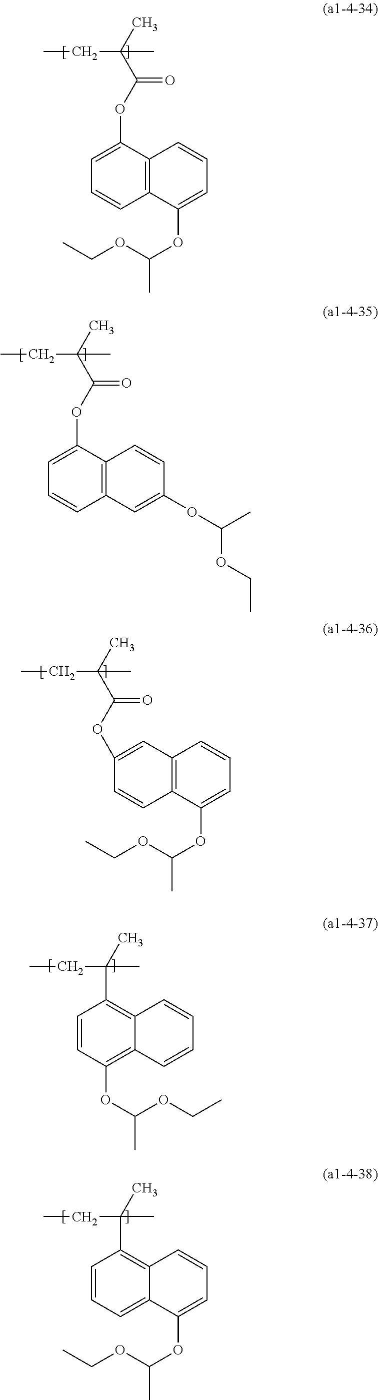

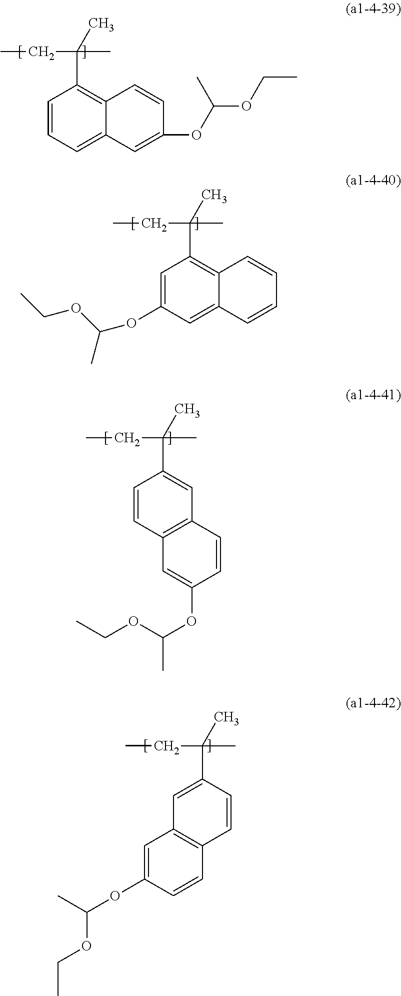

When the resin (A) or the like includes the structural unit (a1-4), the content is typically 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on the total of all structural units of the resin (A) or the like. The total content is also typically 95 mol % or less, preferably 90 mol % or less, more preferably 85 mol % or less, still more preferably 80 mol % or less, yet more preferably 75 mol % or less, further preferably 70 mol % or less, and still further preferably 65 mol % or less, based on all structural units of the resin (A) or the like. Specifically, the content is preferably 10 to 95 mol %, more preferably 15 to 90 mol %, still more preferably 20 to 85 mol %, yet more preferably 20 to 70 mol %, and particularly preferably 20 to 60 mol %.

The structural unit derived from a (meth)acrylic monomer having the group (2) also includes a structural unit represented by a formula (a1-5) (hereinafter may be referred to as “structural unit (a1-5)”).

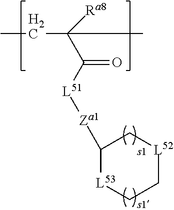

[In the formula (a1-5), all reference numerals are the same as defined above]

Examples of the halogen atom and the alkyl group optionally having a halogen atom in R^{a3} of the formula (a1-5) include those that are the same as described as for R^{a01}, R^{a4}, and R^{a5} of the formula (a1-0), formula (a1-1), and formula (a1-2).

In the formula (a1-5), R^{a3} is preferably a hydrogen atom, a methyl group, or a trifluoromethyl group.

L⁵¹ is preferably an oxygen atom.

One of L⁵² and L⁵³ is preferably —O— and the other one is preferably —S—.

s1 is preferably 1.

s1′ is preferably an integer of 0 to 2.

Z^{a1} is preferably a single bond or *—CH₂—CO—O—.

The structural unit (a1-5) includes, for example, structural units derived from the monomers described in JP-A-2010-61117. Among these structural units, structural units represented by a formula (a1-5-1) to a formula (a1-5-4) are preferable, and structural units represented by the formula (a1-5-1) or a formula (a1-5-2) are more preferable.

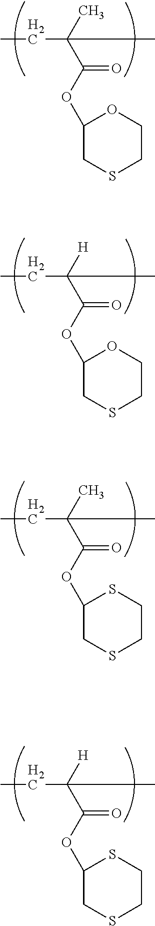

When the resin (A) or the like includes the structural unit (a1-5), the content is typically 1 mol % or more, preferably 2 mol % or more, more preferably 3 mol % or more, still more preferably 5 mol % or more, yet more preferably 10 mol % or more, further preferably 20 mol % or more, and still further preferably 25 mol % or more, based on all structural units of the resin (A) or the like. The content is also typically 80 mol % or less, preferably 70 mol % or less, more preferably 60 mol % or less, still more preferably 50 mol % or less, yet more preferably 45 mol % or less, further preferably 40 mol % or less, and still further preferably 30 mol % or less, based on all structural units of the resin (A) or the like. Specifically, the content is preferably 1 to 50 mol %, more preferably 3 to 45 mol %, still more preferably 5 to 40 mol %, and yet more preferably 5 to 30 mol %, based on all structural units of the resin (A) or the like.

Examples of the structural unit having the group (1) in the structural unit (a1) include a structural unit represented by a formula (a1-6) (hereinafter may be referred to as “structural unit (a1-6)”).

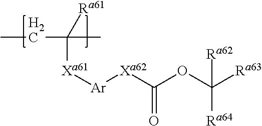

[In the formula (a1-6), all reference numerals are the same as defined above]

Examples of the halogen atom and the alkyl group optionally having a halogen atom in R^{a61} of the formula (a1-6) include those that are the same as described as for R^{a01}, R^{a4}, and R^{a5} of the formula (a1-0), formula (a1-1), and formula (a1-2).

R^{a61} is preferably a hydrogen atom, a methyl group or a trifluoromethyl group.

Examples of the alkyl group in R^{a62}, R^{a63}, R^{a64}, and R^{a65} include a methyl group, an ethyl group, a propyl group, a butyl group, a pentyl group, and a hexyl group. The alkyl group is preferably an alkyl group having 1 to 4 carbon atoms, more preferably an alkyl group having 1 to 3 carbon atoms, and still more preferably a methyl group or an ethyl group.

Examples of the cyclic hydrocarbon group in R^{a62}, R^{a63}, and R^{a64} include an alicyclic hydrocarbon group and an aromatic hydrocarbon group.

The alicyclic hydrocarbon group may be either monocyclic or polycyclic. Examples of the monocyclic alicyclic hydrocarbon group include a cyclopentyl group, a cyclohexyl group, a cycloheptyl group, and a cyclooctyl group. Examples of the polycyclic alicyclic hydrocarbon group include a decahydronaphthyl group, an adamantyl group, and a norbornyl group. The number of carbon atoms of the alicyclic hydrocarbon group is preferably 3 to 16, and more preferably 3 to 12.

Examples of the aromatic hydrocarbon group include a phenylene group, and a naphthylene group.

Examples of the substituent that may be possessed by the cyclic hydrocarbon group include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group.

Examples of the ring formed by bonding R^{a62} and R^{a63} to each other include an adamantane ring, a cyclopentane ring, or a cyclohexane ring. Specifically, when R^{a62} and R^{a63} are bonded to each other to form a ring, examples of —C(R^{a62})(R^{a63})(R^{a64}) include the following groups. * represents a binding site to the oxygen atom. The number of carbon atoms of the ring is preferably 3 to 16, and more preferably 3 to 12.

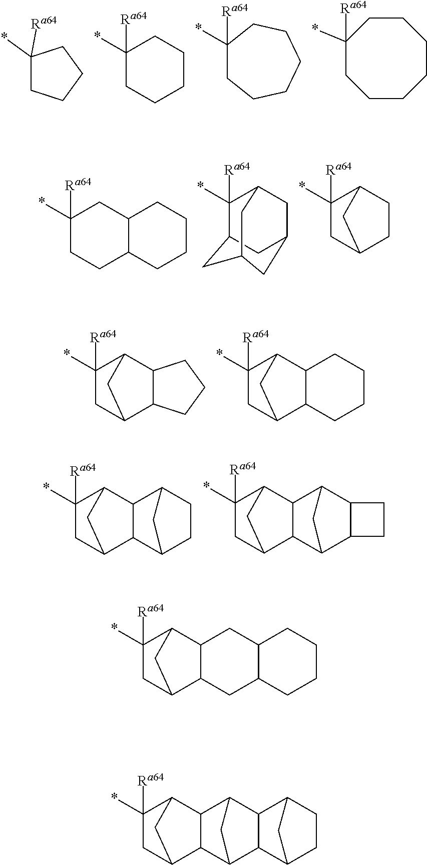

Examples of the alkanediyl group having 1 to 4 carbon atoms in L^{a61} and L^{a62} include a methylene group, an ethylene group, a propane-1,3-diyl group, a propane-1,2-diyl group, a butane-1,4-diyl group, a butane-1,3-diyl group, a 2-methylpropane-1,3-diyl group, and a 2-methylpropane-1,2-diyl group.

Preferably, L^{a61} and L^{a62} each independently is a methylene group or an ethylene group.

X^{a61} is preferably a single bond or —CO—O—*, and more preferably a single bond.

X^{a62} is preferably a single bond or *—O-L^{a61}-, and more preferably a single bond.

Examples of the aromatic hydrocarbon group having 6 to 20 carbon atoms of Ar include a phenylene group, a 1-naphthylene group, a 2-naphthylene group, a 1-anthrylene group, a 9-anthrylene group, a biphenylene group, a 1-phenanthrylene group, and a 2-phenanthrylene group.

Examples of the substituent that may be possessed by the aromatic hydrocarbon group include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group. Examples of these groups include the same groups as those exemplified above. The alkyl group is preferably an alkyl group having 1 to 4 carbon atoms, more preferably a methyl group or an ethyl group, and still more preferably a methyl group. The alkoxy group is preferably an alkoxy group having 1 to 4 carbon atoms, more preferably a methoxy group or an ethoxy group, and still more preferably a methoxy group. The alkoxyalkyl group is preferably an alkoxyalkyl group having 2 to 8 carbon atoms, more preferably a methoxymethyl group or an ethoxyethyl group, and still more preferably a methoxymethyl group. The alkoxyalkoxy group is preferably an alkoxyalkoxy group having 2 to 8 carbon atoms, and more preferably a methoxyethoxy group or an ethoxyethoxy group. The alkylcarbonyl group is preferably an alkylcarbonyl group having 2 to 3 carbon atoms, and more preferably an acetyl group. The alkylcarbonyloxy group is preferably an alkylcarbonyloxy group having 2 to 3 carbon atoms, and more preferably an acetyloxy group.

The substituent is preferably a halogen atom, a hydroxy group, an alkyl group having 1 to 4 carbon atoms, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably a fluorine atom, an iodine atom, a hydroxy group, a methyl group, a methoxy group, or an ethoxyethoxy group.

Ar is preferably a phenylene group optionally having a substituent, and more preferably a phenylene group optionally having a hydroxy group.

Examples of the structural unit (a1-6) include structural units represented by formulas (a1-6-1) to (a1-6-44), and structural units represented by formulas (a1-6-1) to (a1-6-9) and (a1-6-37) to (a1-6-44) are preferable, structural units represented by formulas (a1-6-1), (a1-6-2), (a1-6-4), (a1-6-5), (a1-6-7), (a1-6-8), and (a1-6-37) to (a1-6-44) are more preferable, and structural units represented by formulas (a1-6-7) or (a1-6-8) and (a1-6-37) to (a1-6-44) are still more preferable. It is also possible to exemplify, as the structural unit (a1-6), structural units in which the hydrogen atom corresponding to R^{a61} is substituted with a methyl group or the like, and structural units in which the methyl group corresponding to R^{a61} is substituted with a hydrogen atom or the like.

[chemical structure]

[chemical structure]

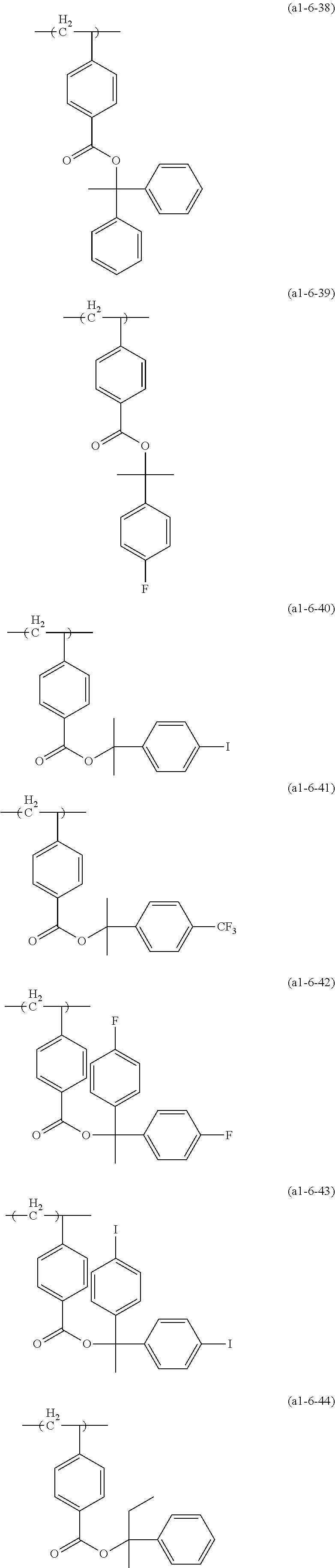

When the resin (A) or the like includes a structural unit (a1-6), the content is usually 3 mol % or more, preferably 5 mol % or more, more preferably 7 mol % or more, still more preferably 10 mol % or more, yet more preferably 20 mol % or more, further preferably 30 mol % or more, and still further preferably 40 mol % or more, based on all structural units of the resin (A) or the like. The content is also typically 80 mol % or less, preferably 75 mol % or less, more preferably 70 mol % or less, and still more preferably 65 mol % or less, based on all structural units of the resin (A). Specifically, the content is preferably 3 to 80 mol %, more preferably 5 to 75 mol %, still more preferably 7 to 70 mol %, yet more preferably 7 to 65 mol %, and particularly preferably 10 to 60 mol %.

Examples of the structural unit (a1) also include the following structural units.

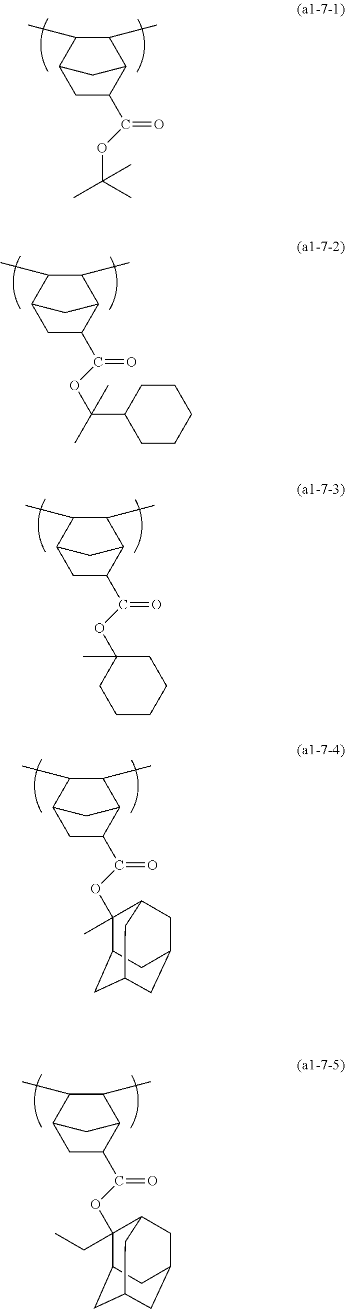

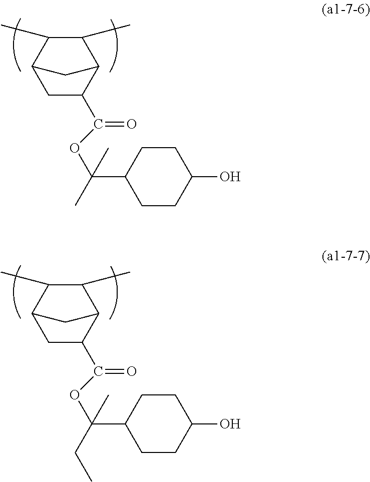

When the resin (A) or the like includes structural units represented by formulas (a1-7-1) to (a1-7-7), the content is usually 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. In addition, the content is also typically 95 mol % or less, preferably 90 mol % or less, more preferably 85 mol % or less, still more preferably 80 mol % or less, yet more preferably 75 mol % or less, further preferably 70 mol % or less, and still further preferably 60 mol % or less. Specifically, the content is preferably 10 to 95 mol %, more preferably 15 to 90 mol %, still more preferably 20 to 85 mol %, yet more preferably 20 to 70 mol %, and particularly preferably 20 to 60 mol %.

Examples of the structural unit (a1) also include the following structural units.

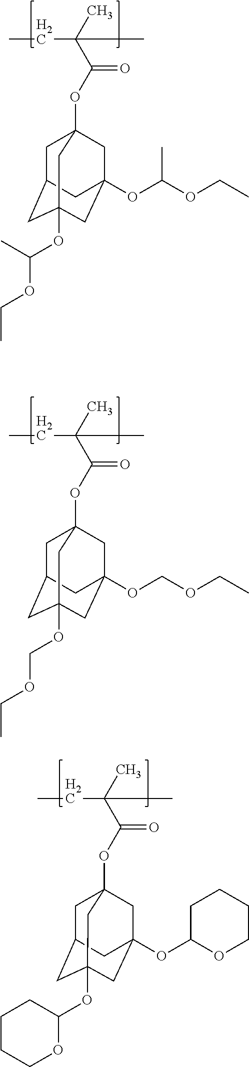

When the resin (A) or the like includes structural units represented by formulas (a1-8-1) to (a1-8-3), the content is typically 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. In addition, the content is also typically 95 mol % or less, preferably 90 mol % or less, more preferably 85 mol % or less, still more preferably 80 mol % or less, yet more preferably 75 mol % or less, further preferably 70 mol % or less, and still further preferably 60 mol % or less. Specifically, the content is preferably 10 to 60 mol %, more preferably 15 to 55 mol %, still more preferably 20 to 50 mol %, yet more preferably 20 to 45 mol %, and particularly preferably 20 to 40 mol %.

<Structural Unit (s)>

The structural unit (s) is derived from a monomer having no acid-labile group (hereinafter may be referred to as “monomer (s)”). It is possible to use, as the monomer from which the structural unit (s) is derived, a monomer having no acid-labile group known in the resist field.

The structural unit (s) preferably has a hydroxy group, a carboxy group, or a lactone ring. When a resin including a structural unit having a hydroxy group or a carboxy group and having no acid-labile group (hereinafter may be referred to as “structural unit (a2)”) and/or a structural unit having a lactone ring and having no acid-labile group (hereinafter may be referred to as “structural unit (a3)”) is used in the resist composition of the present invention, it is possible to improve the resolution of a resist pattern and the adhesion to a substrate. In addition to the structural unit described above, examples of the structural unit (s) include a structural unit having a halogen atom (hereinafter, may be referred to as a “structural unit (a4)”), a structural unit having a non-desorbed hydrocarbon group (hereinafter, may be referred to as a “structural unit (a5)”), a structural unit having a sultone structure (hereinafter, may be referred to as a “structural unit (a6)”), a structural unit that is decomposed by exposure to generate an acid (hereinafter, may be referred to as a “structural unit (a7)”), and other structural units well known in the art.

<Structural Unit (a2)>

The structural unit (a2) is a structural unit represented by a formula (a2) and has an alcoholic hydroxy group, a phenolic hydroxy group, or a carboxy group:

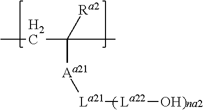

[In the formula (a2),
R^{a2} represents a hydrogen atom, a halogen atom, or an alkyl group having 1 to 6 carbon atoms and optionally having a halogen atom,
A^{a21} represents a single bond or an alkanediyl group having 1 to 12 carbon atoms, and —CH₂— included in the alkanediyl group may be substituted with —O—, —CO—, or —NR^{a28}—,
R^{a28} represents a hydrogen atom or an alkyl group having 1 to 6 carbon atoms,
L^{a21} represents a single bond or a hydrocarbon group having 1 to 28 carbon atoms, the hydrocarbon group may have a substituent, and —CH₂— included in the hydrocarbon group may be substituted with —O—, —CO—, —S—, —SO—, —NR^{a28}—, or —SO₂—.
L^{a22} represents a single bond or a chain hydrocarbon group having 1 to 12 carbon atoms and optionally having a fluorine atom, and —CH₂— included in the chain hydrocarbon group may be substituted with —O—, —CO—, —S—, —SO—, —NR^{a28}—, or —SO₂—.

na2 represents an integer of 1 to 5. When na2 is 2 or more, a plurality of L^{a22} may be the same as or different from each other.]

Examples of the halogen atom and the alkyl group optionally having a halogen atom in R^{a2} of the formula (a2) include the same groups as those exemplified for R^{a01}, R^{a4}, and R^{a5} of the formula (a1-0) and the like.

R^{a2} is preferably a hydrogen atom or an alkyl group having 1 to 4 carbon atoms, more preferably a hydrogen atom, a methyl group or an ethyl group, and still more preferably a hydrogen atom or a methyl group.

Examples of the alkanediyl group of A^{a21} include the same alkanediyl groups as A^{a11} of the formula (a1-4). The number of carbon atoms of the alkanediyl group is preferably 1 to 10, more preferably 1 to 8, still more preferably 1 to 6, yet more preferably 1 to 4, further preferably 1 to 3, and still further preferably 1 or 2.

Examples of the alkyl group as for R^{a28} include a methyl group, an ethyl group, an n-propyl group, an isopropyl group, an n-butyl group, and a tert-butyl group.

A^{a21} is preferably a single bond, *—CO—O— or *—CO—O-A^{a22}-CO—O—, more preferably a single bond, *—CO—O— or *—CO—O—CH₂—CO—O—, and still more preferably a single bond or *—CO—O—. Examples of the alkanediyl group as for A^{a22} include the same alkanediyl group as for A^{a21} as long as the upper limit of the number of carbon atoms permits.

The hydrocarbon group in L^{a21} is an (na2+1)-valent saturated or unsaturated hydrocarbon group, and examples thereof include a linear or branched chain hydrocarbon group, cyclic hydrocarbon groups such as a monocyclic or polycyclic (including a spiro ring, a fused ring or a bridged ring) alicyclic hydrocarbon group and an aromatic hydrocarbon group, and the hydrocarbon group may be a group obtained by combining two or more of these groups (for example, a hydrocarbon group formed from an alicyclic hydrocarbon group or an aromatic hydrocarbon group and a chain hydrocarbon group).

Examples of the chain hydrocarbon group as for L^{a21} include di- to hexa-valent chain hydrocarbon groups such as an alkanediyl group, an alkanetriyl group, an alkanetetrayl group, an alkanepentayl group, and an alkanehexayl group.

Examples of the alkanediyl group include the same alkanediyl groups as for A^{a21}.

Examples of the alkanetriyl group include a methanetriyl group, an ethanetriyl group, a propanetriyl group, a butanetriyl group, a pentanetriyl group, a hexanetriyl group, a heptanetriyl group, an octanetriyl group, a nonanetriyl group, a decanetriyl group, an undecanetriyl group, a dodecanetriyl group, a tridecanetriyl group, a tetradecanetriyl group, a pentadecanetriyl group, a hexadecanetriyl group, and a heptadecanetriyl group.

Examples of the alkanetetrayl group include a methanetetrayl group, an ethanetetrayl group, a propanetetrayl group, a butanetetrayl group, a pentanetetrayl group, a hexanetetrayl group, a heptanetetrayl group, an octanetetrayl group, a nonanetetrayl group, a decanetetrayl group, an undecanetetrayl group, a dodecanetetrayl group, a tridecanetetrayl group, a tetradecanetetrayl group, a pentadecanetetrayl group, a hexadecanetetrayl group, and a heptadecanetetrayl group.

Examples of the alkanepentayl group include an ethanepentayl group, a propanepentayl group, a butanepentayl group, a pentanepentayl group, a hexanepentayl group and the like.

Examples of the alkanehexayl group include an ethanehexayl group, a propanehexayl group, a butanehexayl group, a pentanehexayl group, and a hexanehexayl group.

In addition, examples thereof include groups in which one or more hydrogen atoms of the above-described groups are substituted with a binding site.

The number of carbon atoms of the chain hydrocarbon group is preferably 1 to 18, more preferably 1 to 12, still more preferably 1 to 10, yet more preferably 1 to 9, further preferably 1 to 8, still further preferably 1 to 6, yet still further preferably 1 to 5, and particularly preferably 1 to 4.

Examples of the monocyclic and polycyclic alicyclic hydrocarbon group in L^{a21} include the same groups as those exemplified in the present specification as long as the upper limit of the number of carbon atoms is acceptable.

Examples thereof include di- to hexa-valent alicyclic hydrocarbon groups such as a cycloalkanediyl group, a cycloalkanetriyl group, a cycloalkanetetrayl group, a cycloalkanepentayl group, and a cycloalkanehexayl group.

Specific examples thereof include monocyclic alicyclic hydrocarbon groups such as a cyclobutane-1,3-diyl group, a cyclopentane-1,3-diyl group, a cyclohexane-1,4-diyl group, a cyclooctane-1,5-diyl group, a cyclopropanetriyl group, a cyclobutanetriyl group, a cyclopentanetriyl group, a cyclohexanetriyl group, a cycloheptanetriyl group, a cyclooctanetriyl group, a cyclodecanetriyl group, a cyclopropanetetrayl group, a cyclobutanetetrayl group, a cyclopentanetetrayl group, a cyclohexanetetrayl group, a cycloheptanetetrayl group, a cyclooctanetetrayl group and a cyclodecanetetrayl group, and
polycyclic alicyclic hydrocarbon groups such as a norbornane-1,4-diyl group, a norbornane-2,5-diyl group, an adamantane-1,5-diyl group, an adamantane-2,6-diyl group, a decahydronaphthalenediyl group, a bicyclo[3.3.0]octanediyl group, a norbornanetriyl group, an adamantanetriyl group, a decahydronaphthalenetriyl group, a bicyclo[3.3.0]octanetriyl group, a norbornanetetrayl group, an adamantanetetrayl group, a decahydronaphthalenetetrayl group, and a bicyclo[3.3.0]octanetetrayl group.

In addition, examples thereof include groups in which one or more hydrogen atoms of the above-described groups are substituted with a binding site.

The number of carbon atoms of the alicyclic hydrocarbon group is preferably 3 to 20, more preferably 3 to 18, still more preferably 3 to 16, yet more preferably 3 to 12, further preferably 6 to 12, and particularly preferably 6 to 10.

Examples of the aromatic hydrocarbon group in L^{a21} include di- to hexa-valent aromatic hydrocarbon groups such as an arylene group, an arenetriyl group, an arenetetrayl group, an arenepentayl group, and an arenehexayl group.

Specific examples thereof include a phenylene group, a naphthylene group, an anthrylene group, a biphenylene group, a phenanthrylene group, a benzenetriyl group, a naphthalenetriyl group, an anthracenetriyl group, a biphenylenetriyl group, a phenanthrenetriyl group, a benzenetetrayl group, a naphthalenetetrayl group, an anthracenetetrayl group, a biphenylenetetrayl group, and a phenanthrenetetrayl group. The number of carbon atoms of the aromatic hydrocarbon group is preferably 6 to 20, more preferably 6 to 18, still more preferably 6 to 14, yet more preferably 6 to 12, and further preferably 6 to 10.

In addition, examples thereof include groups in which one or more hydrogen atoms of the above-described groups are substituted with a binding site.

Examples of the group obtained by combining two or more groups include a group obtained by combining an alicyclic hydrocarbon group with a chain hydrocarbon group, a group obtained by combining an aromatic hydrocarbon group with a chain hydrocarbon group, a group obtained by combining an alicyclic hydrocarbon group with an aromatic hydrocarbon group, and a group obtained by combining an alicyclic hydrocarbon group, a chain hydrocarbon group, and an aromatic hydrocarbon group. In combination, two or more of alicyclic hydrocarbon groups, aromatic hydrocarbon groups, and chain hydrocarbon groups may be respectively combined. Any group may be bonded to A^{a21} and L^{a22}.

Examples of the group in which —CH₂— included in the hydrocarbon group is substituted with —O—, —S—, —SO—, —SO₂—, —NR^{a28}—, or —CO— include a hydroxyl group, a carboxyl group, a thiol group, an amino group, an alkoxy group, an alkylthio group, an alkoxycarbonyl group, an alkylsulfonyl group, an alkylamino group, an alkylpeptide group, an alkylcarbonyl group, an alkylcarbonyloxy group, an alkoxycarbonyloxy group, an alkoxyalkoxy group, an oxy group, a carbonyl group, a thio group, a sulfonyl group, a peptide group, an alkanediyloxy group, an alkanediyloxycarbonyl group, an alkanediylcarbonyl group, an alkanediylcarbonyloxy group, an alkanediyloxycarbonyloxy group, an alkanediylsulfonyl group, an alkanediylthio group, an alkanediylamino group,
an alkanediylpeptide group, a cycloalkoxy group, a cycloalkylalkoxy group, an aromatic hydrocarbon group-carbonyloxy group, an aromatic hydrocarbon group-carbonyl group, an aromatic hydrocarbon group-oxy group, and a group obtained by combining two or more of these groups. In addition, examples thereof include groups in which one or more hydrogen atoms of the above-described groups are substituted with a binding site. Examples of these substituted groups include those that are the same as described in the present specification as long as the upper limit of the number of carbon atoms permits.

Examples of the group in which —CH₂— included in the alicyclic hydrocarbon group, the aromatic hydrocarbon group, and the group combining them is substituted with —O—, —S—, —CO—, —SO—, —NR^{a28}—, or —SO₂— include the same groups as those exemplified in the present specification as long as the upper limit of the number of carbon atoms is acceptable.

The hydrocarbon group in L^{a21} may have one or a plurality of substituents. Examples of the substituent include a halogen atom, a haloalkyl group having 1 to 12 carbon atoms, an alkyl group having 1 to 16 carbon atoms (—CH₂— included in the alkyl group may be substituted with —O— or —CO—), an acryloyloxy group or a methacryloyloxy group.

Examples of the halogen atom include a fluorine atom, a chlorine atom, a bromine atom, and an iodine atom.

The hydrocarbon group in L^{a21} can substantially have a substituent such as a haloalkyl group by having a halogen atom as the substituent. Examples of the haloalkyl group include an alkyl fluoride group, an alkyl chloride group, an alkyl bromide group, an alkyl iodide group, for example, a chloromethyl group, a bromomethyl group, an iodomethyl group, a fluoromethyl group, a difluoromethyl group, a trifluoromethyl group, a perfluoroethyl group, a perfluoropropyl group, and a perfluorobutyl group. Among them, a haloalkyl group having 1 to 4 carbon atoms is preferable, and a haloalkyl group having 1 to 3 carbon atoms is more preferable. The number of carbon atoms of the haloalkyl group is preferably 1 to 8, more preferably 1 to 6, still more preferably 1 to 4, and yet more preferably 1 to 3.

The hydrocarbon group as for L^{a21} can substantially have a substituent such as an alkyl group by including a branched structure in L^{a21}. Examples of the alkyl group include a methyl group, an ethyl group, an n-propyl group, an isopropyl group, an n-butyl group, a sec-butyl group, a tert-butyl group, a pentyl group, a hexyl group, a heptyl group, a 2-ethylhexyl group, an octyl group, a nonyl group, a decyl group, an undecyl group, and a dodecyl group. The number of carbon atoms of the alkyl group is preferably 1 to 12, more preferably 1 to 10, still more preferably 1 to 8, yet more preferably 1 to 6, further preferably 1 to 4, and still further preferably 1 to 3.

L^{a21} may substantially have a substituent such as a hydroxy group, a carboxy group, an alkoxy group, an alkoxycarbonyl group, an alkylcarbonyl group, an alkylcarbonyloxy group, a thiol group, an alkylthio group, an alkylsulfonyl group, an alkoxyalkyl group, an alkoxyalkoxy group, an alkoxycarbonyloxy group, an amino group, an alkylamino group, an alkylpeptide group, an acryloyloxy group, or a methacryloyloxy group by a group in which —CH₂— included in the hydrocarbon group of L^{a21} is substituted with —O—, —S—, —CO—, —SO—, —NR^{a28}—, or —SO₂—.

As the substituent that may be possessed by the hydrocarbon group in L^{a21}, a halogen atom, a haloalkyl group having 1 to 8 carbon atoms, or an alkyl group having 1 to 12 carbon atoms (—CH₂— included in the alkyl group may be substituted with —O— or —CO—) is preferable, a halogen atom, a haloalkyl group having 1 to 6 carbon atoms, or an alkyl group having 1 to 10 carbon atoms (—CH₂— included in the alkyl group may be substituted with —O— or —CO—) is more preferable, a halogen atom, a haloalkyl group having 1 to 4 carbon atoms, or an alkyl group having 1 to 6 carbon atoms (—CH₂— included in the alkyl group may be substituted with —O— or —CO—) is still more preferable, a fluorine atom, an iodine atom, a haloalkyl group having 1 to 3 carbon atoms, or an alkyl group having 1 to 4 carbon atoms (—CH₂— included in the alkyl group may be substituted with —O— or —CO—) is still more preferable, and a fluorine atom, an iodine atom, a trifluoromethyl group, a methyl group, a hydroxy group, or a methoxy group is still more preferable.

When the alkanediyl group as for L^{a21} is substituted with —O— or —CO—, for example, it is also preferably *-L^{a23}-X^{a21}. (L^{a23} represents an alkanediyl group having 1 to 8 carbon atoms, X^{a21} represents —O—, —O—CO—, —CO—O— or —O—CO—O—, and * represents a binding site to A^{a21}).

L^{a21} is preferably a single bond, a chain hydrocarbon group having 1 to 12 carbon atoms and optionally having a substituent (in which —CH₂— included in the chain hydrocarbon group may be substituted with —O— or —CO—), a cyclic hydrocarbon group having 3 to 20 carbon atoms and optionally having a substituent (in which —CH₂— included in the cyclic hydrocarbon group may be substituted with —O—, —S—, —CO—, —SO—, NR^{a28}—, or —SO₂—), or groups obtained by combining a chain hydrocarbon group having 1 to 8 carbon atoms and optionally having a substituent with a cyclic hydrocarbon group having 3 to 20 carbon atoms and optionally having a substituent (in which —CH₂— included in the chain hydrocarbon group may be substituted with —O— or —CO—, and —CH₂— included in the cyclic hydrocarbon group may be substituted with —O—, —S—, —SO—, —SO₂—, NR^{a28}—, or —CO—), and more preferably a chain hydrocarbon group having 1 to 10 carbon atoms and optionally having a substituent (in which —CH₂— included in the chain hydrocarbon group may be substituted with —O— or —CO—), a cyclic hydrocarbon group having 3 to 18 carbon atoms and optionally having a substituent (in which —CH₂— included in the cyclic hydrocarbon group may be substituted with —O—, —S—, —CO—, —SO—, —NR^{a28}—, or —SO₂—), or groups obtained by combining a chain hydrocarbon group having 1 to 6 carbon atoms and optionally having a substituent with a cyclic hydrocarbon group having 3 to 18 carbon atoms and optionally having a substituent (in which —CH₂— included in the chain hydrocarbon group may be substituted with —O— or —CO—, and —CH₂— included in the cyclic hydrocarbon group may be substituted with —O—, —S—, —SO—, —SO₂—, —NR^{a28}—, or —CO—).

Examples of the chain hydrocarbon group having 1 to 12 carbon atoms of L^{a22} include a linear alkanediyl group and a branched alkanediyl group, and examples thereof include the same alkanediyl group as A^{a21}. The number of carbon atoms of the chain hydrocarbon group is preferably 1 to 10, more preferably 1 to 8, still more preferably 1 to 6, and yet more preferably 1 to 4.

The number of fluorine atoms possessed by L^{a22} may be either 1, or 2 or more.

—CH₂— included in the chain hydrocarbon group of L^{a22} may be substituted with —O—, —S—, —CO—, —SO—, —NR^{a28}—, or —SO₂—, and these substituted groups are the same as those exemplified for L^{a21} as long as the upper limit of the number of carbon atoms is acceptable.

na2 is preferably an integer of 1 to 4, and more preferably an integer of 1 to 3.

Examples of the structural unit (a2) when L^{a21} is a single bond or a chain hydrocarbon group include the following structural units. Among the following structural units, the structural unit in which the methyl group corresponding to R^{a2} is substituted with a hydrogen atom or the like is preferable structural unit of the structural unit (a2), similarly to the following structural units.

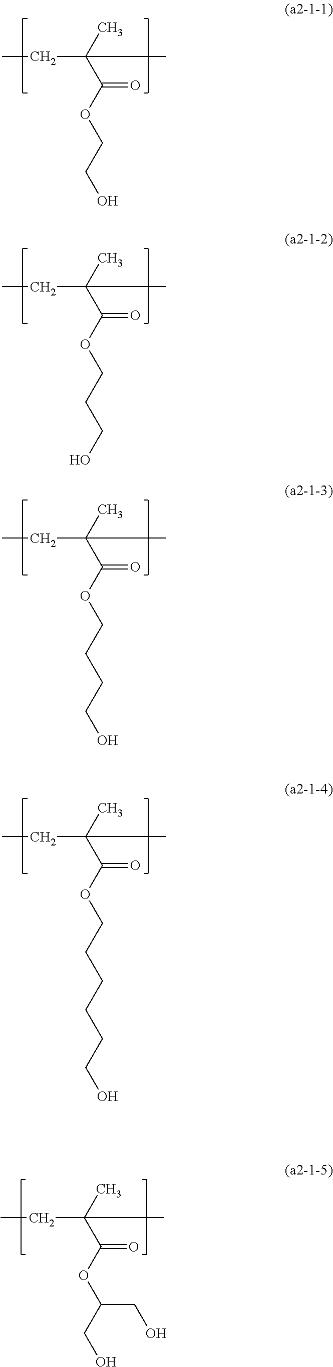

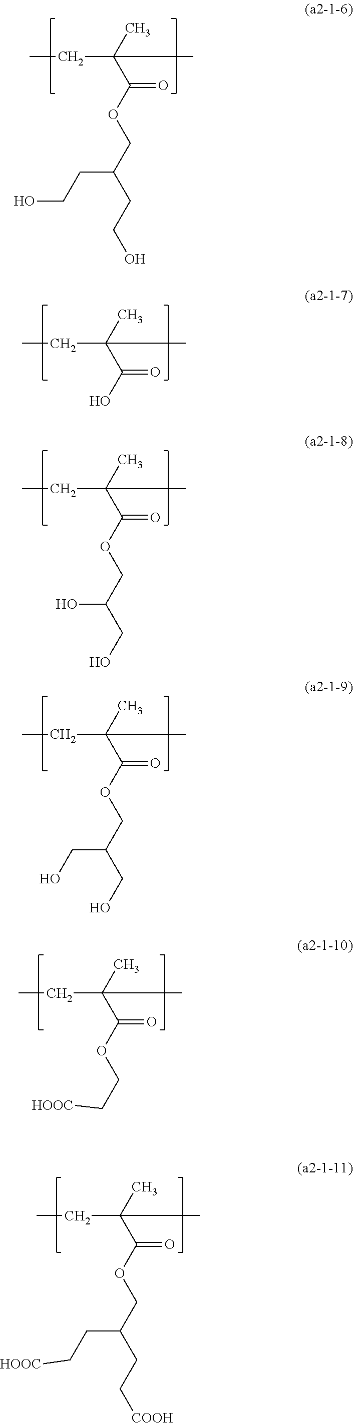

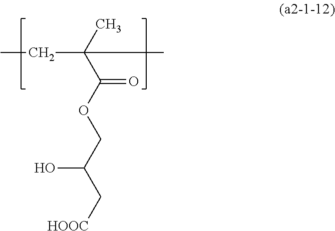

When a resist pattern is produced from the resist composition of the present invention, in the case of using, as an exposure source, high energy rays such as KrF excimer laser (248 nm), electron beam or extreme ultraviolet light (EUV), the structural unit (a2) having a phenolic hydroxy group is preferably used, and the below-described structural unit (a2-A) is more preferably used, as the structural unit (a2). When using ArF excimer laser (193 nm) or the like, the structural unit (a2) having an alcoholic hydroxy group is preferably used, and the below-described structural unit (a2-C) is more preferably used, as the structural unit (a2). The structural unit (a2) may be included singly, or two or more structural units may be included.

In the structural unit (a2), when L^{a21} is a cyclic hydrocarbon group, examples of the structural unit having a phenolic hydroxy group or a carboxy group include a structural unit represented by formula (a2-A) (hereinafter sometimes referred to as “structural unit (a2-A)”).

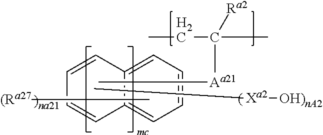

[In the formula (a2-A), all reference numerals are the same as defined above.]

Examples of R^{a2} and A^{a21} include the same groups as described in the formula (a2), respectively.

Examples of R^{a27} include the same groups as those exemplified for R^{a11} of the formula (a1-4).

R^{a27} is preferably a halogen atom, a hydroxy group, a carboxy group, an alkyl group having 1 to 4 carbon atoms and optionally having a halogen atom, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, still more preferably a fluorine atom, an iodine atom, a hydroxy group, a methyl group, a methoxy group, or an ethoxyethoxy group.

nA2 is preferably 1, 2, 3, or 4, more preferably 1, 2, or 3, and still more preferably 1 or 2.

na21 is preferably 0, 1, 2, or 3, more preferably 0, 1, or 2, and still more preferably 0 or 1.

mc is preferably 0 or 1.

In the case of the benzene ring, —X^{a2}—OH may be bonded at the o-position, the in-position, or the p-position, with respect to the binding site of A^{a21}. In particular, at least one is preferably bonded at the in-position or the p-position, and more preferably bonded at the m-position. In the case of the naphthalene ring, when the binding site of A^{a21} is the 1-position, it may be bonded at any of the 2-position to the 8-position, and when the binding site of A^{a21} is the 2-position, it may be bonded at any of the 1-position and the 3-position to the 8-position. In particular, when the binding site of A^{a21} is the 1-position, at least one is preferably bonded at the 3-position to the 6-position, and more preferably bonded at the 3-position or the 4-position. When the binding site of A^{a21} is the 2-position, it is preferably bonded at the 4-position to the 7-position, and more preferably bonded at the 5-position or the 6-position.

Examples of the structural unit (a2-A) include structural units derived from the monomers described in JP-A-2010-204634 and JP-A-2012-12577.

Examples of the structural unit (a2-A) include structural units represented by a formula (a2-2-1) to a formula (a2-2-32), structural units represented by a formula (a2-3-1) to a formula (a2-3-24), and structural units in which a methyl group corresponding to R^{a2} in the structural unit (a2-A) is substituted with a hydrogen atom, a halogen atom, a haloalkyl group, or other alkyl groups in structural units represented by the formula (a2-2-1) to the formula (a2-2-32) and structural units represented by the formula (a2-3-1) to the formula (a2-3-24).

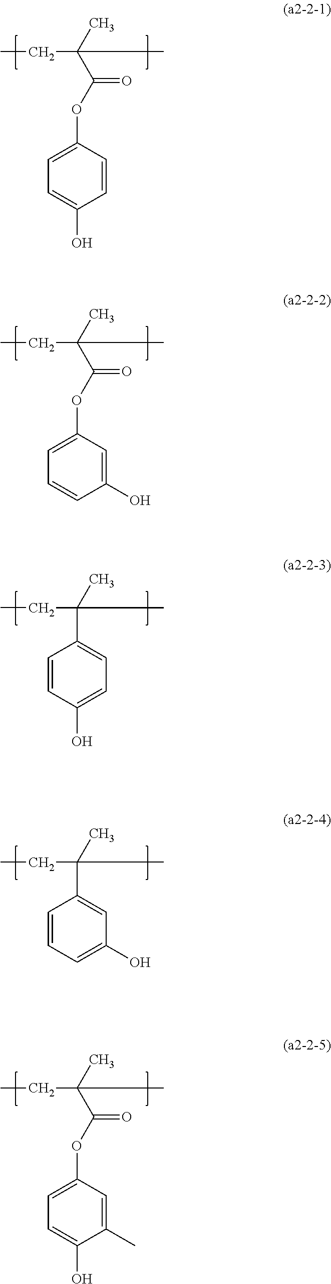

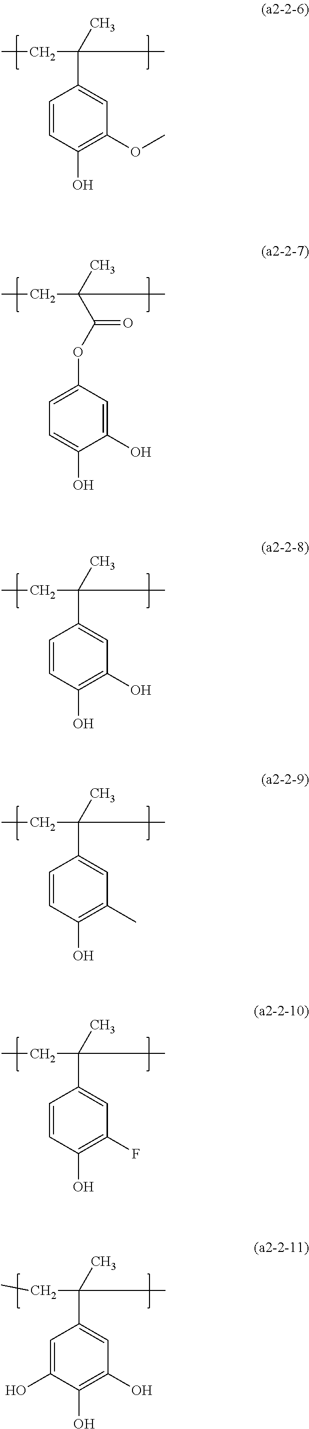

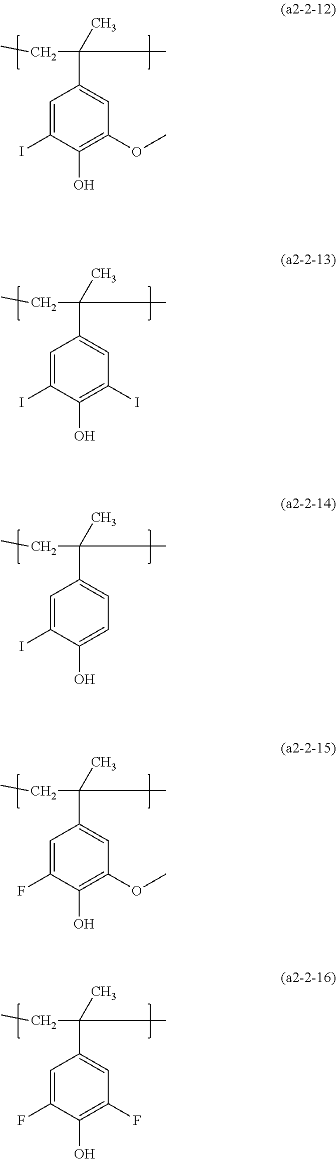

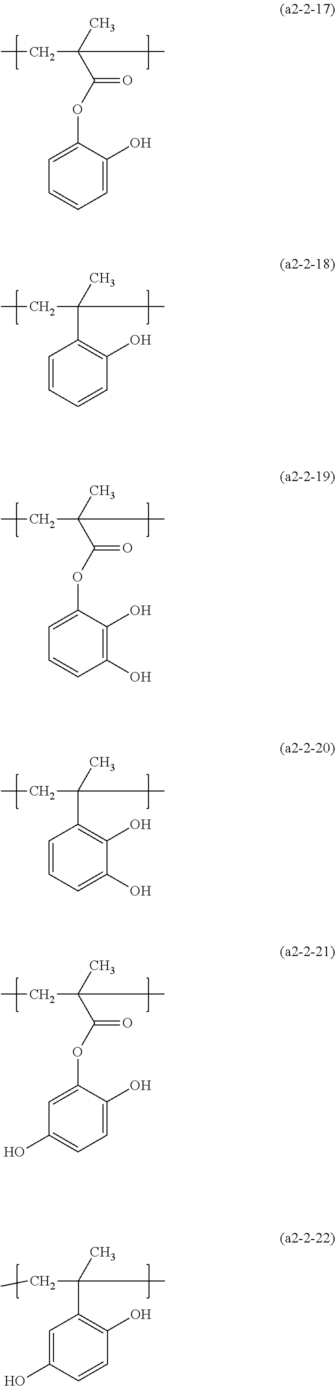

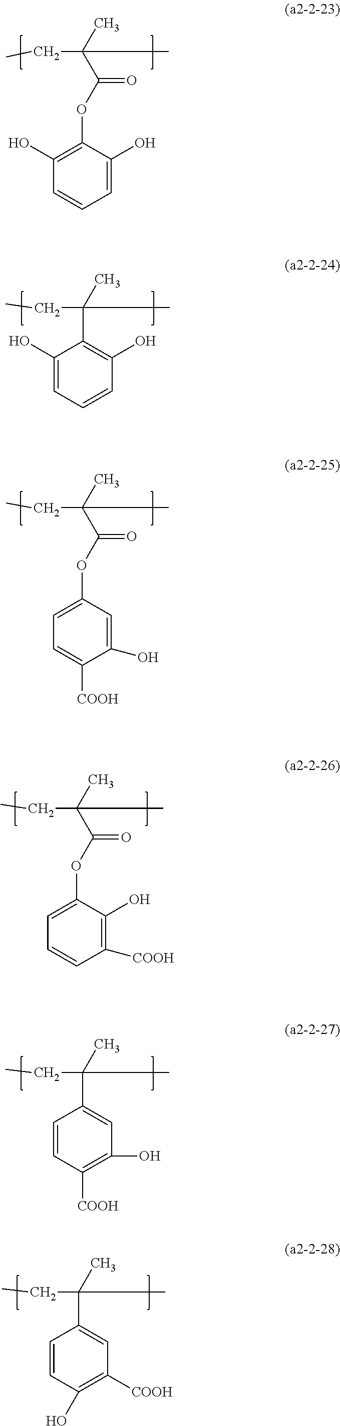

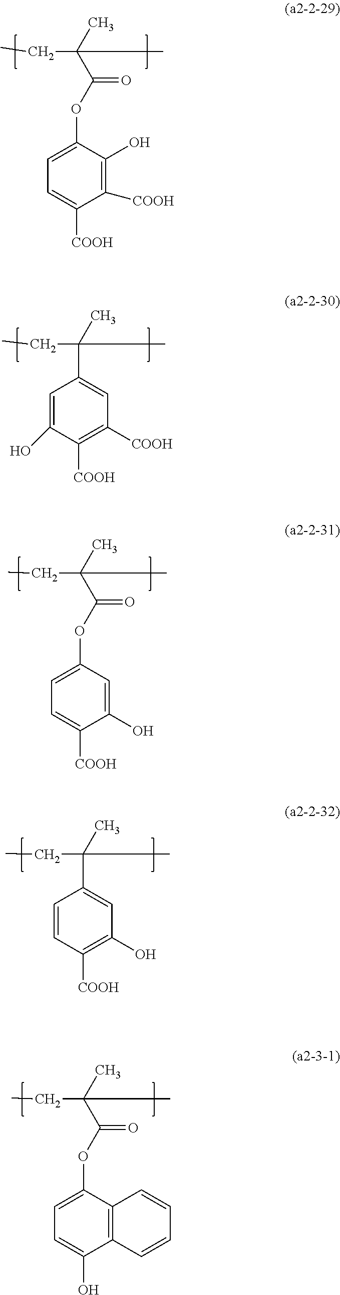

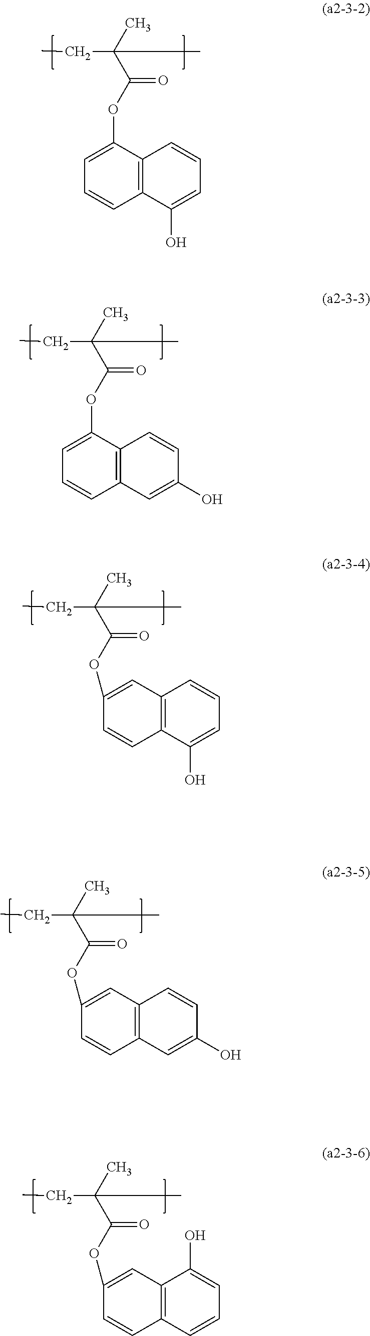

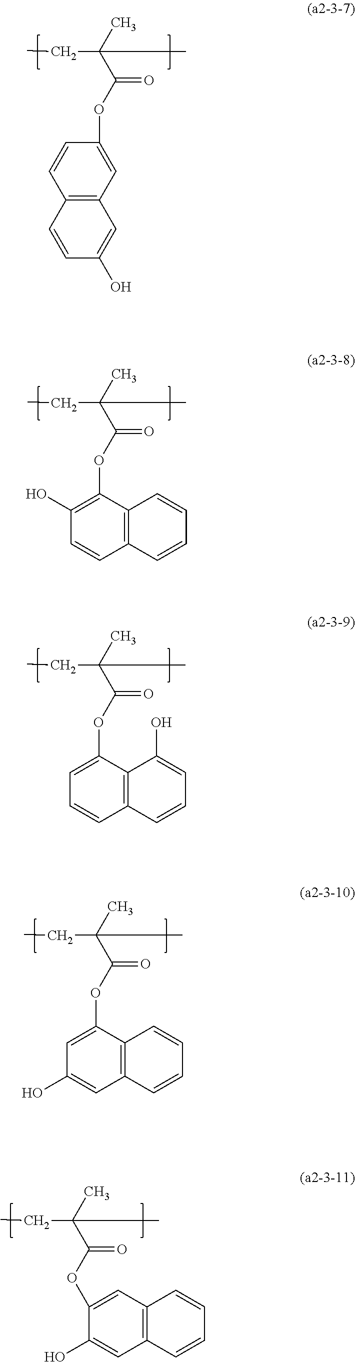

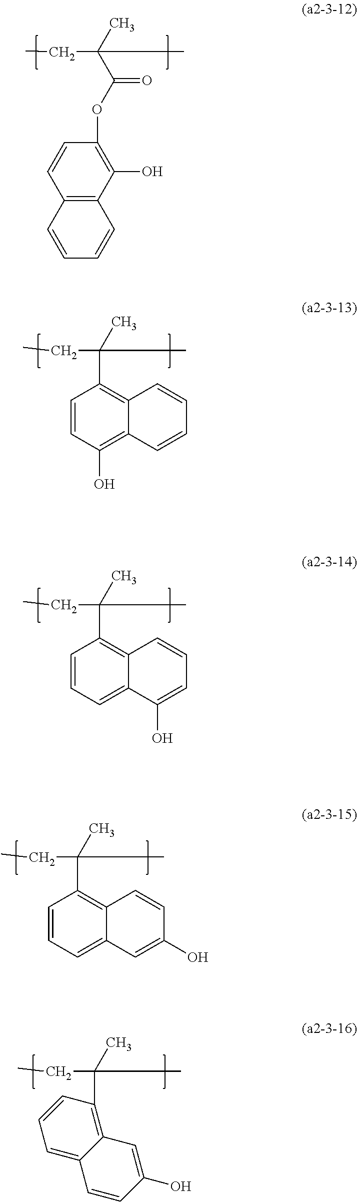

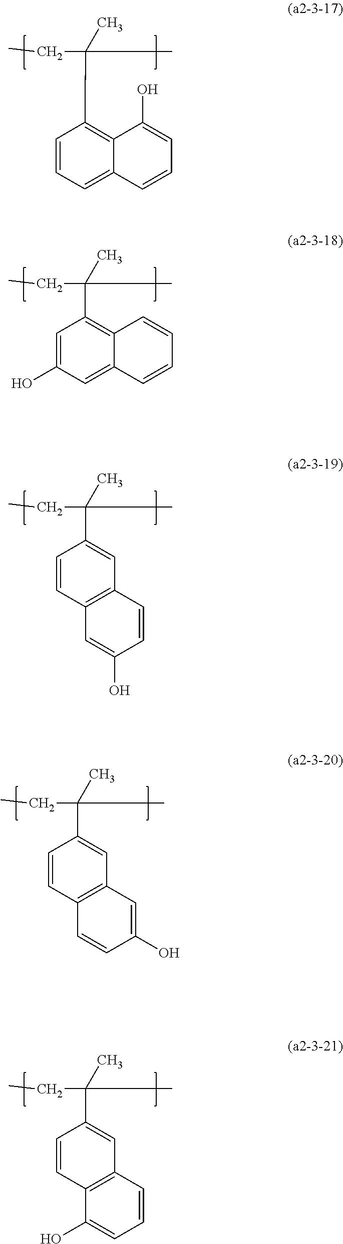

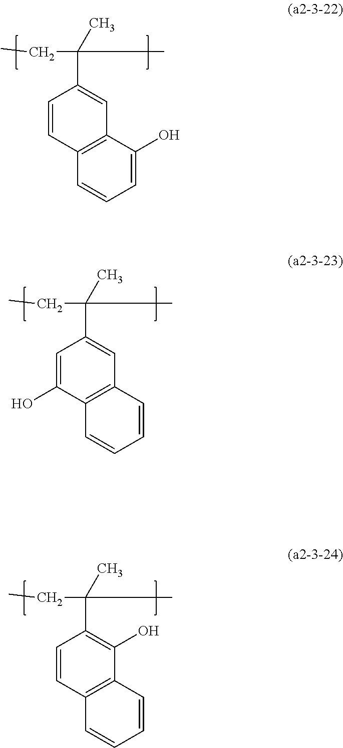

When the structural unit (a2-A) is included in the resin (A) or the like, the content of the structural unit (a2-A) is preferably 5 mol % or more, more preferably 10 mol % or more, still more preferably 15 mol % or more, and yet more preferably 20 mol % or more, based on all structural units. In addition, the content is preferably 80 mol % or less, more preferably 70 mol % or less, still more preferably 65 mol % or less, yet more preferably 60 mol % or less, further preferably 50 mol % or less, still further preferably 45 mol % or less, and yet further preferably 40 mol % or less. Specifically, the content is preferably 5 to 80 mol %, more preferably 10 to 70 mol %, still more preferably 15 to 65 mol %, and yet more preferably 20 to 65 mol %.

The structural unit (a2-A) is derived from a compound represented by a formula (a2-A′) (hereinafter, may be referred to as “compound (a2-A)”).

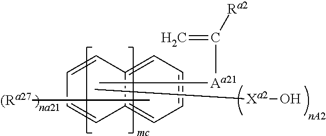

[In the formula (a2-A′), all reference numerals are the same as defined above.]

The compound (a2-A′) may be a commercially available product or may be produced by a known method.

The structural unit (a2-A) can be included in the resin (A) or the like by polymerizing the compound (a2-A′). In addition, the structural unit (a2-A) can be included in the resin (A) by polymerizing, for example, with a structural unit (a1-4) and treating with an acid such as p-toluenesulfonic acid. In addition, the structural unit (a2-A) can be included in the resin (A) or the like by polymerizing with acetoxystyrene and treating with an alkali such as tetramethylammonium hydroxide.

In the structural unit (a2), when L^{a21} is a cyclic hydrocarbon group, examples of the structural unit having an alcoholic hydroxy group or a carboxy group include a structural unit represented by a formula (a2-B) (hereinafter may be referred to as “structural unit (a2-B)”), a structural unit represented by a formula (a2-C) (hereinafter may be referred to as “structural unit (a2-C)”) and a structural unit represented by a formula (a2-D) (hereinafter may be referred to as “structural unit (a2-D)”).

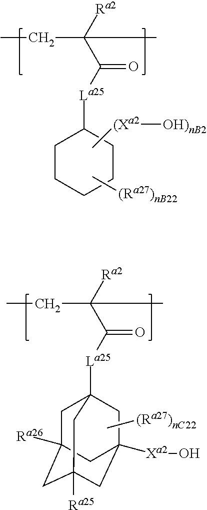

[In the formula (a2-B) and formula (a2-C),
R^{a2} is the same as defined in the formula (a2).

R^{a27} is the same as defined in the formula (a2-A).

L^{a25} represents —O— or *—O—(CH₂)ₖ₂—CO—O—, k2 represents an integer of 1 to 7. * represents a binding site to —CO—.

X^{a2} represents a single bond or —CO—.

R^{a2z} and R^{a2z} each independently represent a hydrogen atom, a methyl group, or a hydroxy group.

nB2 represents an integer of 1 to 5. When nB2 is 2 or more, a plurality of X^{a2} may be the same as or different from each other.

nB22 represents an integer of 0 to 8. When nB22 is 2 or more, a plurality of R^{a27} may be the same as or different from each other.

nC22 represents an integer of 0 to 10. When nC22 is 2 or more, a plurality of R^{a27} may be the same as or different from each other.]

L^{a25} is preferably —O— or —O—(CH₂)_{f1}—CO—O— (f1 represents an integer of 1 to 4), and more preferably —O—.

R^{a2} is preferably a methyl group.

X^{a2} is preferably a single bond.

R^{a2z} is preferably a hydrogen atom.

R^{a2z} is preferably a hydrogen atom or a hydroxy group.

R^{a27} is preferably a halogen atom, a hydroxy group, a carboxy group, an alkyl group having 1 to 4 carbon atoms and optionally having a halogen atom, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, still more preferably a fluorine atom, an iodine atom, a hydroxy group, a methyl group, a methoxy group, or an ethoxyethoxy group.

nB2 is preferably 1, 2, 3 or 4, more preferably 1, 2 or 3, and still more preferably 1 or 2.

nB22 is preferably an integer of 0 to 3, more preferably 0, 1 or 2, and still more preferably 0 or 1.

nC22 is preferably an integer of 0 to 6, more preferably an integer of 0 to 3, still more preferably 0, 1, or 2, and yet more preferably 0 or 1.

Examples of the structural unit (a2-B) and the structural unit (a2-C) include a structural unit derived from the monomer described in JP-A-2010-204646, the following structural units, and structural units in which the methyl group or the hydrogen atom corresponding to R^{a2} in the following structural units is substituted with a hydrogen atom, a halogen atom, a haloalkyl group, or other alkyl groups. Among these, a structural unit represented by any one of a formula (a2-B-1) to a formula (a2-B-5) and a formula (a2-C-1) to a formula (a2-C-9) is preferable.

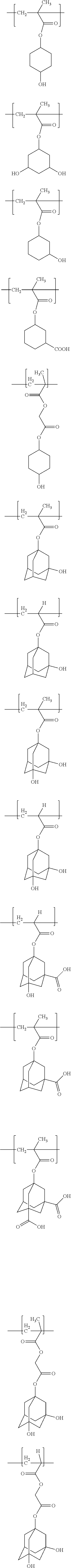

When the resin (A) or the like includes the structural unit (a2-B) or the structural unit (a2-C), the content is typically 1 mol % or more, and preferably 2 mol % or more, based on all structural units of the resin (A) or the like. In addition, the content is typically 45 mol % or less, preferably 40 mol % or less, more preferably 35 mol % or less, still more preferably 20 mol % or less, and yet more preferably 10 mol % or less. Specifically, the content is typically 1 to 45 mol %, preferably 1 to 40 mol %, more preferably 1 to 35 mol %, still more preferably 1 to 20 mol %, and yet more preferably 1 to 10 mol %.

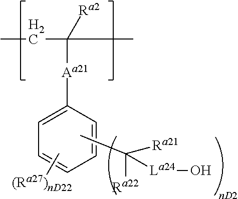

[In the formula (a2-D),

R^{a2} and A^{a21} are respectively the same as defined in the formula (a2).

R^{a27} is the same as defined in the formula (a2-A).

R^{a21} and R^{a22} each independently represent an alkyl fluoride group having 1 to 4 carbon atoms.

L^{a24} represents a single bond or an alkanediyl group having 1 to 3 carbon atoms, and the alkanediyl group may be substituted with a fluorine atom.

nD2 represents an integer of 1 to 5. When nD2 is 2 or more, a plurality of groups in parentheses may be the same as or different from each other.

nD22 represents an integer of 0 to 4. When nD22 is 2 or more, a plurality of R^{a27} may be the same as or different from each other.

Provided that 1≤nD2+nD22≤5]

Examples of the alkyl fluoride group as for R^{a21} and R^{a22} each independently include a trifluoromethyl group, a 2,2,2-trifluoroethyl group, a 3,3,3-trifluoropropyl group, and a 4,4,4-trifluorobutyl group.

R^{a21} and R^{a22} are preferably a trifluoromethyl group.

Examples of the alkanediyl group as for L^{a24} include a methylene group, an ethane-1,1-diyl group, a propane-1,1-diyl group, and a propane-2,2-diyl group.

L^{a24} is preferably a single bond or a methylene group.

R^{a27} is preferably a halogen atom, a hydroxy group, a carboxy group, an alkyl group having 1 to 4 carbon atoms and optionally having a halogen atom, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, still more preferably a fluorine atom, an iodine atom, a hydroxy group, a methyl group, a methoxy group, or an ethoxyethoxy group.

nD2 is preferably 1, 2, 3 or 4, more preferably 1, 2 or 3, still more preferably 1 or 2, and yet more preferably 1. Preferably, nD2 is 1, and a group in parentheses is bonded at the para-position.

nD22 is preferably an integer of 0 to 3, more preferably an integer of 0 to 2, still more preferably 0 or 1, and yet more preferably 0.

The structural unit (a2-D) is more preferably a structural unit represented by the following formula (a2-D1) (hereinafter, may be referred to as “structural unit (a2-D1)”).

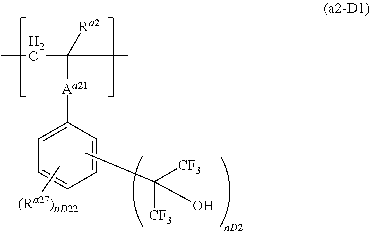

[In the formula (a2-D1),
R^{a2}, A^{a21}, R^{a27}, nD2, and nD22 are the same as defined in the formula (a2-D).]

In the formula (a2-D1), R^{a2} is preferably a hydrogen atom or a methyl group.

A^{a21} is preferably a single bond.

R^{a27} is preferably a halogen atom, a hydroxy group, a carboxy group, an alkyl group having 1 to 4 carbon atoms, a haloalkyl group having 1 to 4 carbon atoms, an alkoxy group having 1 to 4 carbon atoms, or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, still more preferably a fluorine atom, an iodine atom, a hydroxy group, a methyl group, a methoxy group, or an ethoxyethoxy group.

nD22 is preferably 0, 1, 2, or 3, more preferably 0, 1, or 2, and still more preferably 0 or 1.

nD2 is preferably 1, 2, 3, or 4, more preferably 1, 2, or 3, and still more preferably 1 or 2.

Examples of the structural unit (a2-D) include structural units described below.

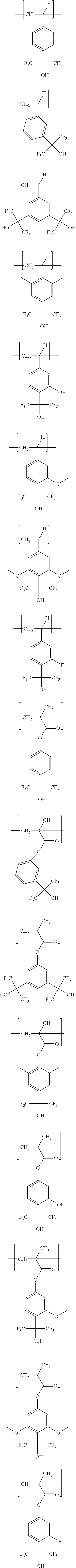

It is possible to exemplify structural units in which the hydrogen atom corresponding to R^{a2} is substituted with a methyl group or the like in structural units represented by a formula (a2-D-1) to a formula (a2-D-8), and structural units in which the methyl group corresponding to R^{a2} is substituted with a hydrogen atom or the like in structural units represented by a formula (a2-D-9) to a formula (a2-D-16) as specific examples of the structural unit (a2-D). Among these, structural units represented by a formula (a2-D-1) to a formula (a2-D-8) are preferable, structural units represented by a formula (a2-D-1) to a formula (a2-D-4) are more preferable, and a structural unit represented by a formula (a2-D-1) is still more preferable.

When the resin (A) or the like includes the structural unit (a2-D), the content is preferably 3 mol % or more, more preferably 5 mol % or more, and still more preferably 10 mol % or more, based on all structural units of the resin (A) or the like. In addition, the content is preferably 80 mol % or less, more preferably 75 mol % or less, still more preferably 70 mol % or less, and yet more preferably 65 mol % or less. Specifically, the content is preferably 3 to 80 mol %, more preferably 5 to 75 mol %, still more preferably 10 to 70 mol %, and yet more preferably 10 to 65 mol %.

<Structural Unit (a3)>

The lactone ring possessed by the structural unit (a3) may be a monocyclic ring such as a B-propiolactone ring, a y-butyrolactone ring or a 5-valerolactone ring, or a fused ring of a monocyclic lactone ring and the other ring. Preferably, a y-butyrolactone ring, an adamantanelactone ring or a bridged ring including a y-butyrolactone ring structure (for example, a structural unit represented by the following formula (a3-2)) is exemplified.

The structural unit (a3) is preferably a structural unit represented by a formula (a3-1), a formula (a3-2), a formula (a3-3), or a formula (a3-4). These structural units may be included singly, or two or more structural units may be included.

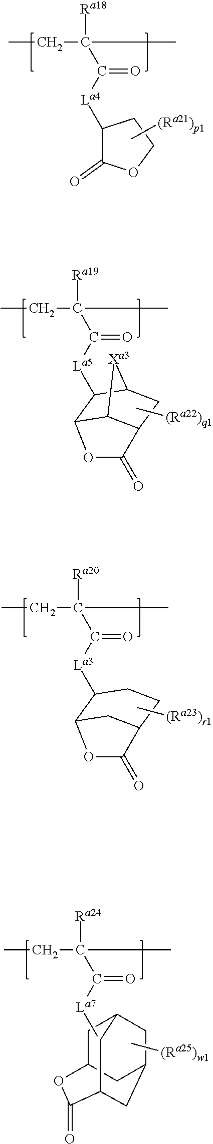

[In the formula (a3-1), formula (a3-2), formula (a3-3), and formula (a3-4),

L^{a4}, L^{a5} and L^{a6} each independently represent —O— or a group represented by *—O—(CH₂)ₖ₃—CO—O— (k3 represents an integer of 1 to 7).

L^{a7} represents —O—, *—O-L^{a8}-O—, *—O-L^{a8}-CO—O—, *—O-L^{a8}-CO—O-L^{a9}-CO— O— or, *—O-L^{a8}-O—CO-L^{a9}-O—.

L^{a3} and L^{a9} each independently represent an alkanediyl group having 1 to 6 carbon atoms.

* represents a binding site to a carbonyl group.

R^{a18}, R^{a19}, R^{a20}, and R^{a24} each independently represent a hydrogen atom, a halogen atom, or an alkyl group having 1 to 6 carbon atoms and optionally having a halogen atom.

X^{a3} represents —CH₂— or an oxygen atom.

R^{a21} represents an aliphatic hydrocarbon group having 1 to 4 carbon atoms.

R^{a22}, R^{a23}, and R^{a25} each independently represent a carboxy group, a cyano group, or an aliphatic hydrocarbon group having 1 to 4 carbon atoms.

p 1 represents an integer of 0 to 5.

q1 represents an integer of 0 to 3.

r1 represents an integer of 0 to 3.

w1 represents an integer of 0 to 8.

When p1, q1, r1, and/or w1 are 2 or more, a plurality of R^{a21}, R^{a22}, R^{a23}, and/or R^{a25} may be the same as or different from each other]

Examples of the aliphatic hydrocarbon group in R^{a21}, R^{a22}, R^{a23}, and R^{a25} include alkyl groups such as a methyl group, an ethyl group, a propyl group, an isopropyl group, a butyl group, a sec-butyl group, and a tert-butyl group.

Examples of the halogen atom and the alkyl group optionally having a halogen atom in R^{a18}, R^{a19}, R^{a20}, and R^{a24} include the same groups as those exemplified for R^{a01}, R^{a4}, and R^{a5} in the formulas (a1-0) to (a1-2).

Examples of the alkanediyl group in L^{a3} and L^{a9} include a methylene group, an ethylene group, a propane-1,3-diyl group, a propane-1,2-diyl group, a butane-1,4-diyl group, a pentane-1,5-diyl group, a hexane-1,6-diyl group, a butane-1,3-diyl group, a 2-methylpropane-1,3-diyl group, a 2-methylpropane-1,2-diyl group, a pentane-1,4-diyl group, and a 2-methylbutane-1,4-diyl group.

Preferably, L^{a4} to L^{a6} are each independently —O— or a group in which k3 is an integer of 1 to 4 in *—O—(CH₂)ₖ₃—CO—O—, more preferably —O— and *—O— CH₂—CO—O—, and still more preferably an oxygen atom.

L^{a7} is preferably —O— or *—O-L^{a8}-CO—O—, and more preferably —O—, —O— CH₂—CO—O—, or —O—C2H₄—CO—O—.

R^{a18}, R^{a19}, R^{a20}, and R^{a24} are preferably a hydrogen atom or an alkyl group having 1 to 4 carbon atoms, more preferably a hydrogen atom, a methyl group or an ethyl group, and still more preferably a hydrogen atom or a methyl group.

R^{a21} is preferably a methyl group.

Preferably, R^{a22}, R^{a23}, and R^{a25} are each independently a carboxy group, a cyano group or a methyl group.

Preferably, p1, q1, and r1 are each independently an integer of 0 to 2, and more preferably 0 or 1.

w1 is preferably an integer of 0 to 2, and more preferably 0 or 1.

Particularly, the formula (a3-4) is preferably a formula (a3-4′).

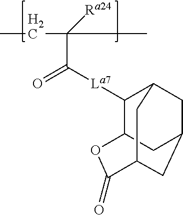

(In the formula, R^{a24} and L^{a7} are the same as defined above.)

Examples of the structural unit (a3) include structural units derived from the monomers described in JP-A-2010-204646, the monomers described in JP-A-2000-122294 and the monomers described in JP-A-2012-41274. The structural unit (a3) is preferably a structural unit represented by any one of a formula (a3-1-1), a formula (a3-1-2), a formula (a3-2-1), a formula (a3-2-2), a formula (a3-3-1), a formula (a3-3-2), and a formula (a3-4-1) to a formula (a3-4-12), and structural units in which methyl groups corresponding to R^{a18}, R^{a19}, R^{a20}, and R^{a24} in the formula (a3-1) to the formula (a3-4) are substituted with hydrogen atoms in the above structural units.

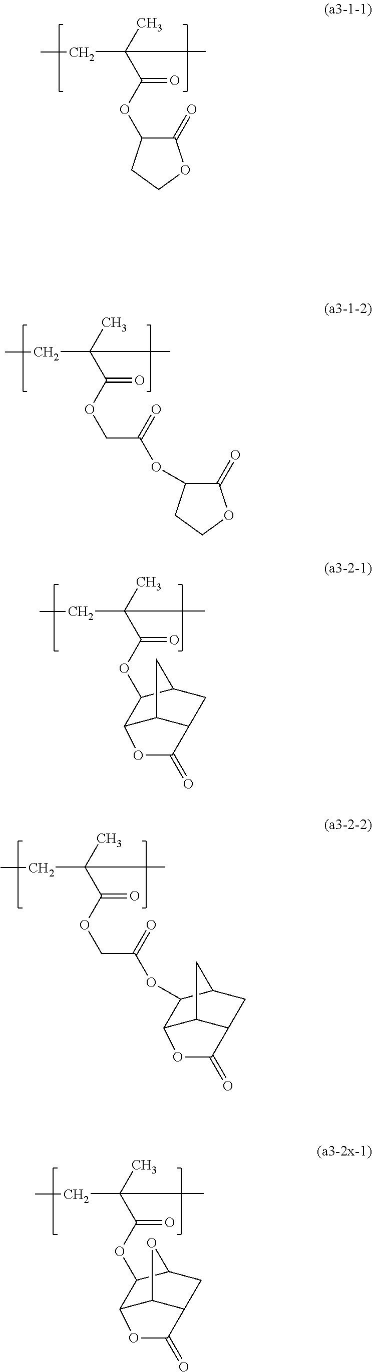

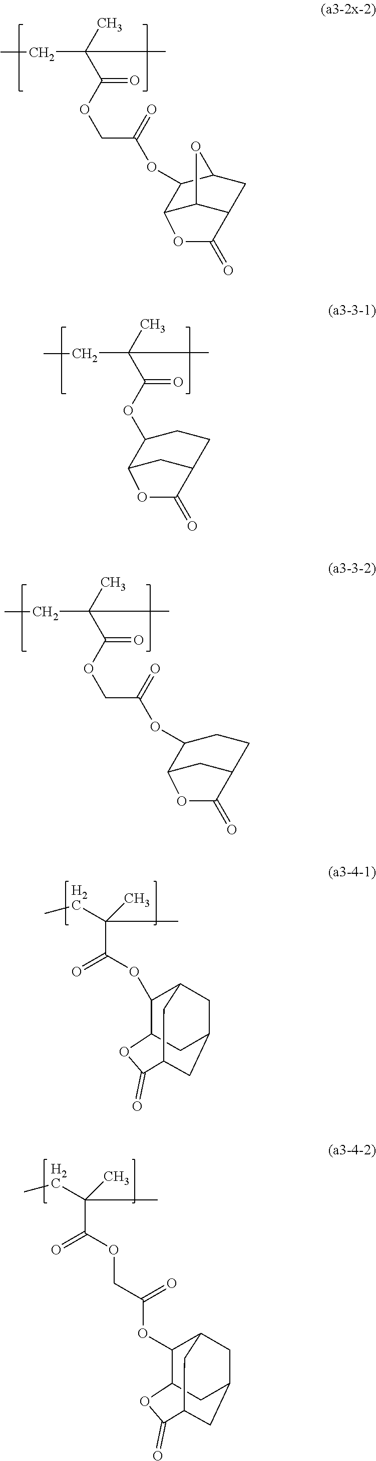

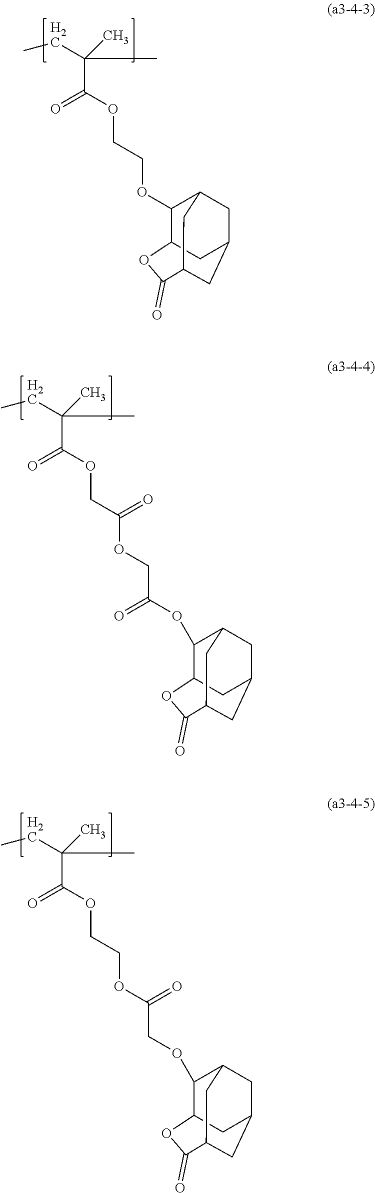

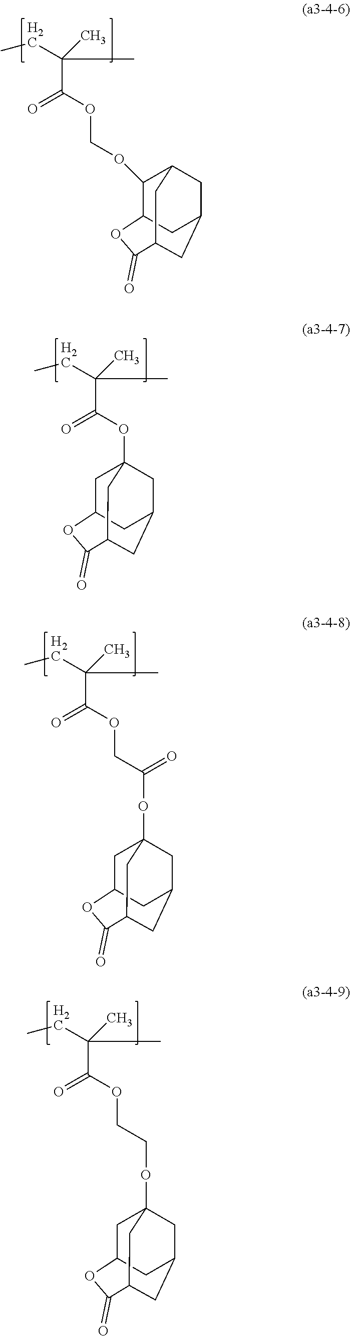

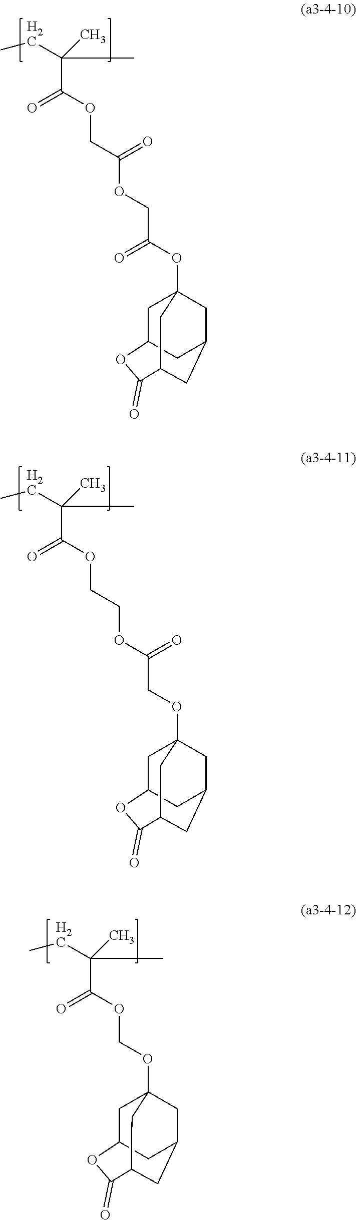

When the resin (A) or the like includes the structural unit (a3), the total content is typically 1 mol % or more, preferably 3 mol % or more, more preferably 5 mol % or more, and still more preferably 10 mol % or more, based on all structural units of the resin (A) or the like. In addition, the total content is typically 70 mol % or less, preferably 65 mol % or less, more preferably 60 mol % or less, still more preferably 50 mol % or less, yet more preferably 40 mol % or less, further preferably 30 mol % or less, still further preferably 25 mol % or less, and yet further preferably 20 mol % or less. Specifically, the total content is typically 1 to 70 mol %, preferably 3 to 70 mol %, more preferably 3 to 65 mol %, still more preferably 5 to 65 mol %, yet more preferably 5 to 60 mol %, and further preferably 10 to 60 mol %.

In addition, each content of the structural unit (a3-1), the structural unit (a3-2), the structural unit (a3-3), or the structural unit (a3-4) is preferably 1 mol % or more, more preferably 3 mol % or more, still more preferably 5 mol % or more, and yet more preferably 10 mol % or more, based on all structural units of the resin (A) or the like. In addition, the content is preferably 60 mol % or less, more preferably 55 mol % or less, still more preferably 50 mol % or less, further preferably 40 mol % or less, still further preferably 30 mol % or less, yet further preferably 25 mol % or less, and yet still further preferably 20 mol % or less. Specifically, the content is preferably 1 to 60 mol %, more preferably 3 to 60 mol %, still more preferably 3 to 55 mol %, yet more preferably 5 to 55 mol %, further preferably 5 to 50 mol %, and still further preferably 10 to 50 mol %.

<Structural Unit (a4)>

The structural unit represented by the formula (a4) is shown below.

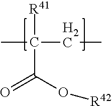

[In the formula (a4),
R⁴¹ represents a hydrogen atom or a methyl group.

R⁴² represents an aliphatic hydrocarbon group having 1 to 48 carbon atoms and having a halogen atom, and —CH₂— included in the aliphatic hydrocarbon group may be substituted with —O— or —CO—.

Examples of the aliphatic hydrocarbon group represented by R⁴² include a linear or branched chain hydrocarbon group and a monocyclic or polycyclic alicyclic hydrocarbon group, and groups formed by combining these groups.

Examples of the chain hydrocarbon group include linear alkyl groups such as a methyl group, an ethyl group, a propyl group, a butyl group, a pentyl group, a hexyl group, a heptyl group, an octyl group, a decyl group, a dodecyl group, a pentadecyl group, a hexadecyl group, a heptadecyl group and an octadecyl group, and branched alkyl groups such as an isopropyl group and an isobutyl group.

Examples of the monocyclic or polycyclic alicyclic hydrocarbon group include: monocyclic alicyclic hydrocarbon groups that are monocyclic cycloalkyl groups such as a cyclopentyl group, a cyclohexyl group, a cycloheptyl group, and a cyclooctyl group; and polycyclic alicyclic hydrocarbon groups that are polycyclic cycloalkyl groups such as a decahydronaphthyl group, an adamantyl group, a norbornyl group, and the following groups (* represents a binding site).

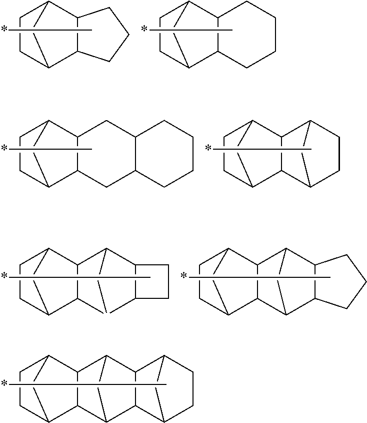

Examples of the groups formed by combination include groups formed by combining one or more alkyl groups or one or more alkanediyl groups with one or more alicyclic hydrocarbon groups, for example, an -alkanediyl group-alicyclic hydrocarbon group, an -alicyclic hydrocarbon group-alkyl group, an -alkanediyl group-alicyclic hydrocarbon group-alkyl group.

Examples of the alkanediyl group include: linear alkanediyl groups such as a methylene group, an ethylene group, a propane-1,3-diyl group, a butane-1,4-diyl group, a pentane-1,5-diyl group, and a hexane-1,6-diyl group; and branched alkanediyl groups such as a propane-1,2-diyl group, a butane-1,3-diyl group, a 2-methylpropane-1,2-diyl group, a 1-methylbutane-1,4-diyl group, and a 2-methylbutane-1,4-diyl group. The end of the branched alkanediyl group may be a methyl group.

Examples of the halogen atom possessed by the aliphatic hydrocarbon group included in R⁴² include a fluorine atom, a chlorine atom, a bromine atom, or an iodine atom.

Examples of the group in which —CH₂— of the aliphatic hydrocarbon group included in R⁴² is substituted with —O— or —CO— include a hydroxy group, a carboxy group, a carbonyl group, an oxy group, an alkoxy group, an alkoxycarbonyl group, an alkylcarbonyl group, an alkylcarbonyloxy group, an alkanediyloxy group, an alkanediyloxycarbonyl group, an alkanediylcarbonyl group, an alkanediylcarbonyloxy group, a cycloalkoxy group, a cycloalkylalkoxy group, and groups obtained by combining two or more of these groups. Examples of these substituted groups include those that are the same as described in the present specification as long as the upper limit of the number of carbon atoms permits. The number of carbon atoms in these substituted groups is, for example, 1 to 35, 1 to 17, preferably 1 to 11, more preferably 1 to 6, still more preferably 1 to 4, and still more preferably 1 to 3 in the case of an alkoxy group or the like, 2 to 35, 2 to 17, preferably 2 to 11, more preferably 2 to 6, still more preferably 2 to 4, and still more preferably 2 or 3 in the case of an alkoxycarbonyl group or the like, 2 to 36, 2 to 18, preferably 2 to 12, more preferably 2 to 6, still more preferably 2 to 4, and still more preferably 2 or 3 in the case of an alkylcarbonyl group or the like, and 2 to 34, 2 to 16, preferably 2 to 10, more preferably 2 to 6, and still more preferably 2 to 4, and yet more preferably 2 or 3 in the case of an alkoxycarbonyloxy group or the like.

The structural unit (a4) is preferably a structural unit in which R⁴² is an aliphatic hydrocarbon group having a fluorine atom, and examples of the structural unit (a4) in which R⁴² is an aliphatic hydrocarbon group having a fluorine atom include a structural unit represented by a formula (a4-1) (hereinafter, may be referred to as “structural unit (a4-1)”), a structural unit represented by a formula (a4-2) (hereinafter, may be referred to as “structural unit (a4-2)”), a structural unit represented by a formula (a4-3) (hereinafter, may be referred to as “structural unit (a4-3)”) and a structural unit represented by a formula (a4-A) (hereinafter, may be referred to as “structural unit (a4-A)”).

The structural unit represented by the formula (a4-1) is a structural unit represented by the following.

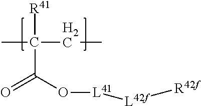

[In the formula (a4-1),
R⁴¹ represents a hydrogen atom or a methyl group.

L⁴¹ represents a single bond or an alkanediyl group having 1 to 4 carbon atoms.

L^{42f} represents an alkanediyl group having 1 to 8 carbon atoms and having a fluorine atom, or a cycloalkanediyl group having 3 to 12 carbon atoms and having a fluorine atom.

R^{42f} represents a hydrogen atom or a fluorine atom]

Examples of the alkanediyl group in L⁴¹ include: linear alkanediyl groups such as a methylene group, an ethylene group, a propane-1,3-diyl group and a butane-1,4-diyl group; and branched alkanediyl groups such as an ethane-1,1-diyl group, a propane-1,2-diyl group, a butane-1,3-diyl group, a 2-methylpropane-1,3-diyl group, and a 2-methylpropane-1,2-diyl group. The end of the branched alkanediyl group may be a methyl group.

Examples of the alkanediyl group having a fluorine atom in L^{42f} include: linear alkanediyl groups having a fluorine atom, such as a methylene group having a fluorine atom, an ethylene group having a fluorine atom, a propane-1,3-diyl group having a fluorine atom, a butane-1,4-diyl group having a fluorine atom, a pentane-1,5-diyl group having a fluorine atom, a hexane-1,6-diyl group having a fluorine atom, a heptane-1,7-diyl group having a fluorine atom, and an octane-1,8-diyl group having a fluorine atom; and
branched alkanediyl group having a fluorine atom, such as an ethane-1,1-diyl group having a fluorine atom, a propane-1,1-diyl group having a fluorine atom, a propane-1,2-diyl group having a fluorine atom, a propane-2,2-diyl group having a fluorine atom, a pentane-2,4-diyl group having a fluorine atom, a 2-methylpropane-1,3-diyl group having a fluorine atom, a 2-methylpropane-1,2-diyl group having a fluorine atom, a pentane-1,4-diyl group having a fluorine atom, and a 2-methylbutane-1,4-diyl group having a fluorine atom. The end of the branched alkanediyl group may be a methyl group optionally having a fluorine atom.

Examples of cycloalkanediyl group having a fluorine atom in L^{42f} include: monocyclic cycloalkanediyl groups having a fluorine atom, such as a cyclobutane-1,3-diyl group having a fluorine atom, a cyclopentane-1,3-diyl group having a fluorine atom, a cyclohexane-1,4-diyl group having a fluorine atom, a cyclohexene-3,6-diyl group having a fluorine atom, a cycloheptane-1,4-diyl group having a fluorine atom and a cyclooctane-1,5-diyl group having a fluorine atom; and
polycyclic cycloalkanediyl groups having a fluorine atom, such as a norbornane-1,4-diyl group having a fluorine atom, a norbornane-2,5-diyl group having a fluorine atom, a 5-norbornene-2,3-diyl group having a fluorine atom, an adamantane-1,5-diyl group having a fluorine atom, and an adamantane-2,6-diyl group having a fluorine atom.

The number of fluorine atoms of the alkanediyl group and the cycloalkanediyl group each having a fluorine atom in L^{42f} may be 1 or more, and preferably 2 or more.

The alkanediyl group and the cycloalkanediyl group each having a fluorine atom in L^{42f} are preferably a perfluoroalkanediyl group and a perfluorocycloalkanediyl group, respectively.

Examples of the perfluoroalkanediyl group in L^{42f} include a difluoromethylene group, a perfluoroethylene group, a perfluoroethylfluoromethylene group, a perfluoropropane-1,3-diyl group, a perfluoropropane-1,2-diyl group, a perfluoropropane-2,2-diyl group, a perfluorobutane-1,4-diyl group, a perfluorobutane-2,2-diyl group, a perfluorobutane-1,2-diyl group, a perfluoropentane-1,5-diyl group, a perfluoropentane-2,2-diyl group, a perfluoropentane-3,3-diyl group, a perfluorohexane-1,6-diyl group, a perfluorohexane-2,2-diyl group, a perfluorohexane-3,3-diyl group, a perfluoroheptane-1,7-diyl group, a perfluoroheptane-2,2-diyl group, a perfluoroheptane-3,4-diyl group, a perfluoroheptane-4,4-diyl group, a perfluorooctane-1,8-diyl group, a perfluorooctane-2,2-diyl group, a perfluorooctane-3,3-diyl group, and a perfluorooctane-4,4-diyl group.

Examples of the perfluorocycloalkanediyl group in L^{42f} include a perfluorocyclohexanediyl group, a perfluorocyclopentanediyl group, a perfluorocycloheptanediyl group, and a perfluoroadamantanediyl group.

L⁴¹ is preferably a single bond or an alkanediyl group having 1 to 3 carbon atoms, more preferably a single bond, a methylene group, or an ethylene group, and still more preferably a single bond, or a methylene group.

L^{42f} is preferably a perfluoroalkanediyl group having 1 to 8 carbon atoms or a perfluorocycloalkanediyl group having 3 to 12 carbon atoms, more preferably a perfluoroalkanediyl group having 1 to 6 carbon atoms, and still more preferably a perfluoroalkanediyl group having 1 to 3 carbon atoms.

Examples of the structural unit (a4-1) include the following structural units, and structural units in which a methyl group corresponding to R⁴¹ in the structural unit (a4-1) in the following structural units is substituted with a hydrogen atom.

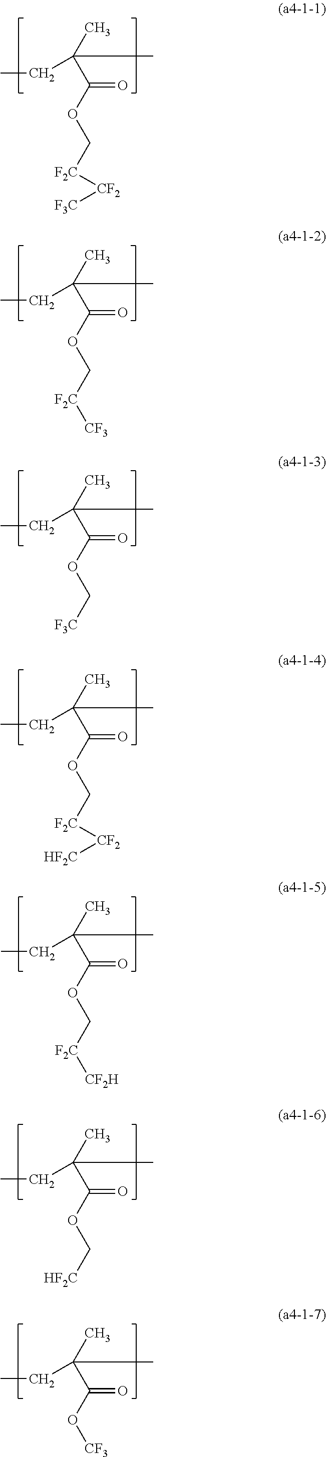

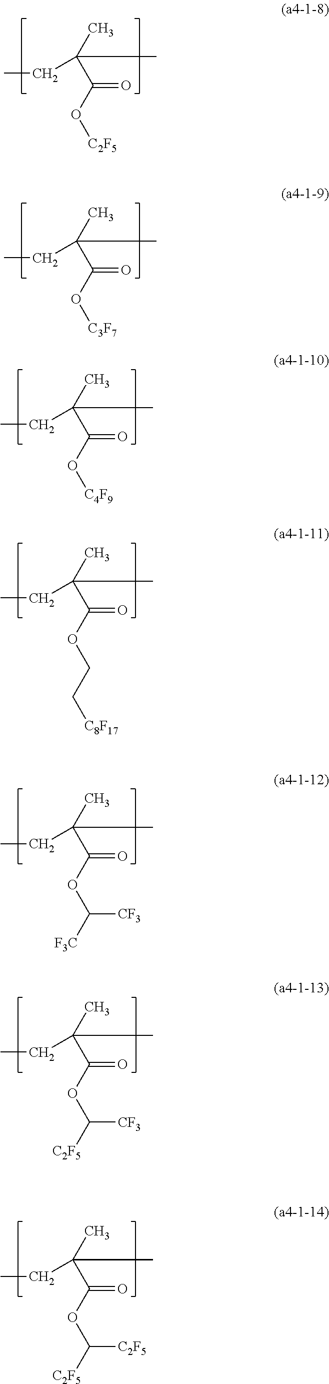

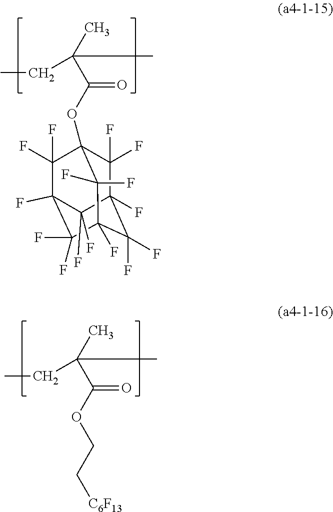

The structural unit represented by the formula (a4-2) is a structural unit represented by the following.

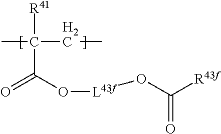

[In the formula (a4-2),
R⁴¹ represents a hydrogen atom or a methyl group.

L^{43f} represents an aliphatic hydrocarbon group having 1 to 20 carbon atoms and optionally having a fluorine atom, and —CH₂— included in the aliphatic hydrocarbon group may be substituted with —O— or —CO—.

R^{43f} represents an aliphatic hydrocarbon group having 1 to 20 carbon atoms and optionally having a fluorine atom, and —CH₂— included in the aliphatic hydrocarbon group may be substituted with —O— or —CO—.

Provided that at least one of L^{43f} and R^{43f} has a fluorine atom, and the upper limit of the total number of carbon atoms of L^{43f} and R^{43f} is 21]

Examples of the aliphatic hydrocarbon group in L^{43f} and R^{43f} include a linear or branched chain hydrocarbon group and a monocyclic or polycyclic alicyclic hydrocarbon group, and groups formed by combining these groups. Examples of the aliphatic hydrocarbon group in L^{43f} and R^{43f} include groups that are the same as the aliphatic hydrocarbon groups described as the aliphatic hydrocarbon group as for R⁴² in the formula (a4) as long as the upper limit of the total number of carbon atoms of L^{43f} and R^{43f} is acceptable.

The aliphatic hydrocarbon group as for L^{43f} is preferably an alkanediyl group having 1 to 6 carbon atoms or a group represented by a formula (L43f-1).

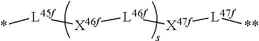

[In the formula (L43f-1),
s represents 0 or 1.

L^{45f} and L^{46f} each independently represent a divalent aliphatic hydrocarbon group having 1 to 5 carbon atoms and optionally having a fluorine atom,

L^{47f} represents a single bond or a divalent aliphatic hydrocarbon group having 1 to 5 carbon atoms and optionally having a fluorine atom.

X^{46f} and X^{47f} each independently represent —O—, —CO—, —CO—O—, or —O— CO—.

Provided that the total number of carbon atoms of L^{45f}, L^{46f}, L^{47f}, X^{46f}, and X^{47f} is 7 or less.

* and ** are binding sites, and ** is a binding site to —O—CO—R^{43f}]

Examples of the divalent aliphatic hydrocarbon group represented by L^{45f}, L^{46f}, and L^{47f} in the group represented by the formula (L43f-1) include a linear or branched alkanediyl group and a monocyclic or polycycic divalent alicyclic hydrocarbon group, and a divalent aliphatic hydrocarbon group formed by combining an alkanediyl group and a divalent alicyclic hydrocarbon group. Specific examples thereof include a methylene group, an ethylene group, a propane-1,3-diyl group, a propane-1,2-diyl group, a butane-1,4-diyl group, a 1-methylpropane-1,3-diyl group, a 2-methylpropane-1,3-diyl group, and a 2-methylpropane-1,2-diyl group.

s is preferably 0.

Examples of the group represented by the formula (L43f-1) include the following groups. In the following, * and ** each represent a binding site, and ** is a binding site to —O—CO—R^{43r}.

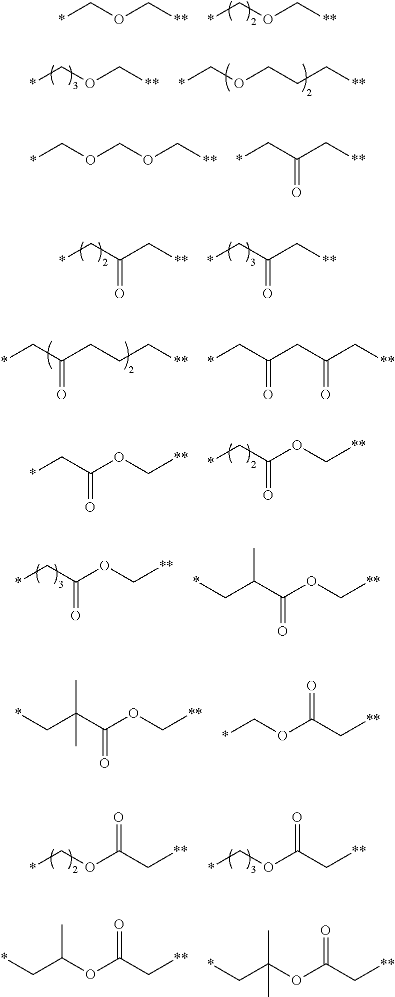

R^{43f} is preferably an aliphatic hydrocarbon group having a fluorine atom.

In this case, examples of the structural unit represented by the formula (a4-2) include a structural unit represented by a formula (a4-2A) or a formula (a4-2B).

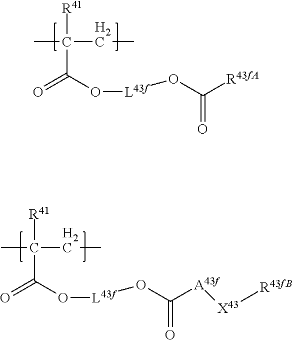

[In the formula (a4-2A) and the formula (a4-2B),
R⁴¹ and L^{43f} are the same as defined in the formula (a4-2).

R^{43fA} represents an aliphatic hydrocarbon group having 1 to 20 carbon atoms and having a fluorine atom.

A^{43f} represents a divalent aliphatic hydrocarbon group having 1 to 17 carbon atoms and optionally having a fluorine atom, and —CH₂— included in the aliphatic hydrocarbon group may be substituted with —O— or —CO—.

X⁴³ represents **—O—CO— or **—CO—O—, and ** represents a binding site to A^{43f}.

R^{43fB} represents an aliphatic hydrocarbon group having 1 to 17 carbon atoms and optionally having a fluorine atom.

Provided that in the formula (a4-2A), the total number of carbon atoms of L^{43f} and R^{43f} is 21 or less, and in the formula (a4-2B), the total number of carbon atoms of L^{43f}, A^{43f}, X⁴³, and R^{43fB} is 21 or less, and at least one of A^{43f} and R^{43fB} has at least one fluorine atom.

Examples of the aliphatic hydrocarbon group as for R^{43fA} include the same aliphatic hydrocarbon groups as described as for R⁴² in the formula (a4) as long as the upper limit of the number of carbon atoms is acceptable.

R^{43fA} is preferably an alkyl group having 1 to 13 carbon atoms and having a fluorine atom, a cycloalkyl group having 3 to 12 carbon atoms and having a fluorine atom, or group obtained by combining these groups, and more preferably *—(CF₂)_{n43f}—R^{42′} (* represents a binding site to a carbonyl group, n43f represents an integer of 1 to 6, and R^{42′} represents a hydrogen atom or a fluorine atom) or a perfluoro cycloalkyl group having 3 to 12 carbon atoms.

Examples of the aliphatic hydrocarbon group as for A^{43f} and R^{43fB} include the same aliphatic hydrocarbon groups as described as for R⁴² in the formula (a4) as long as the upper limit of the number of carbon atoms is acceptable.

A^{43f} is preferably a divalent chain hydrocarbon group and optionally having a fluorine atom, a divalent alicyclic hydrocarbon group, or groups obtained by combining these groups, more preferably a divalent chain hydrocarbon group having a fluorine atom, and still more preferably an alkanediyl fluoride group having 1 to 6 carbon atoms.

The aliphatic hydrocarbon group optionally having a fluorine atom as for R^{43fB} is preferably a chain hydrocarbon group, an alicyclic hydrocarbon group, or groups obtained by combining these groups optionally having a fluorine atom, more preferably an alkyl group having 1 to 12 carbon atoms, a cycloalkyl group having 3 to 12 carbon atoms, or groups obtained by combining these groups optionally having a fluorine atom, and still more preferably alkyl fluoride groups such as a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a heptyl group, a perfluoro heptyl group, an octyl group and a perfluorooctyl group; a cyclopropylmethyl group, a cyclopropyl group, a cyclobutylmethyl group, a cyclopentyl group, a cyclohexyl group, a perfluoro cyclohexyl group, an adamantyl group, an adamantylmethyl group, an adamantyldimethyl group, a norbornyl group, a norbornylmethyl group, a perfluoroadamantyl group, and a perfluoroadamantylmethyl group.

In the formula (a4-2B), an example of the structure that the group represented by *-A^{43f}-X⁴³-R^{43fB} can take includes the following structures (* is a binding site to a carbonyl group).

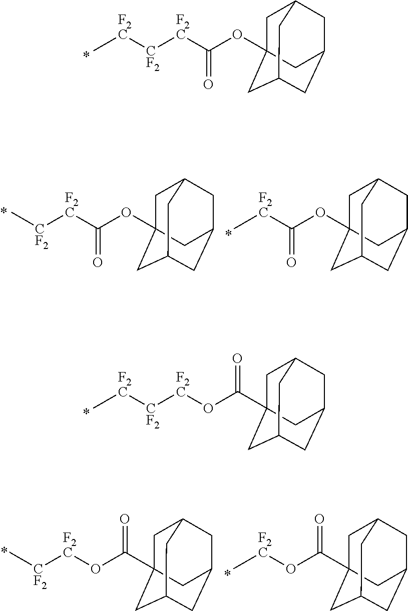

Examples of the structural unit represented by the formula (a4-2A) include the following structural units, and structural units in which the methyl group corresponding to R⁴¹ in the structural unit represented by the formula (a4-2A) in the following structural units is substituted with a hydrogen atom.

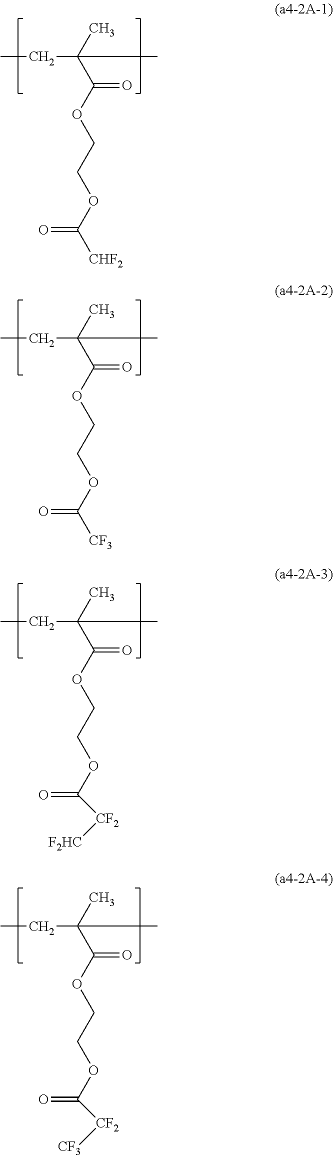

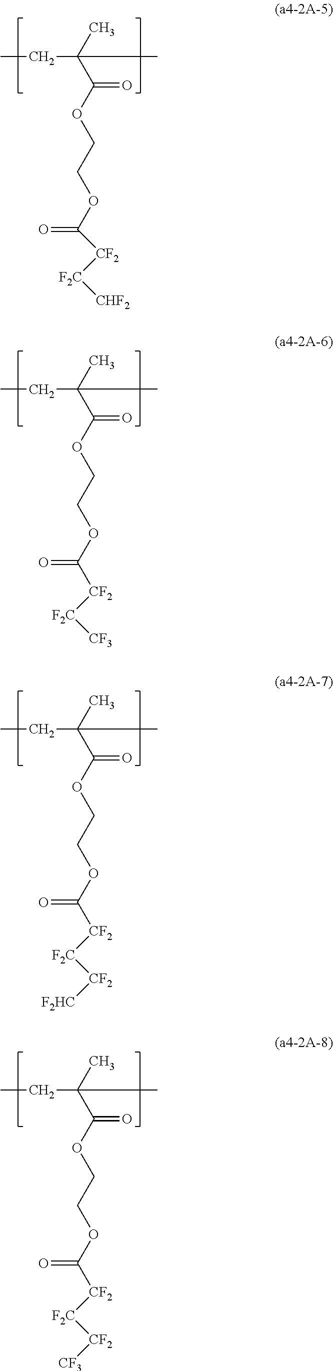

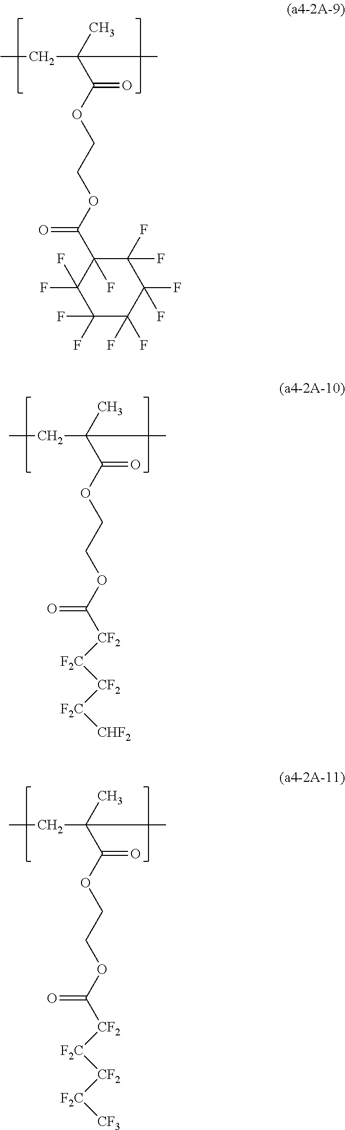

Examples of the structural unit represented by the formula (a4-2B) include the following structural units, and structural units in which the methyl group corresponding to R⁴¹ in the structural unit represented by the formula (a4-2B) in the following structural units is substituted with a hydrogen atom.

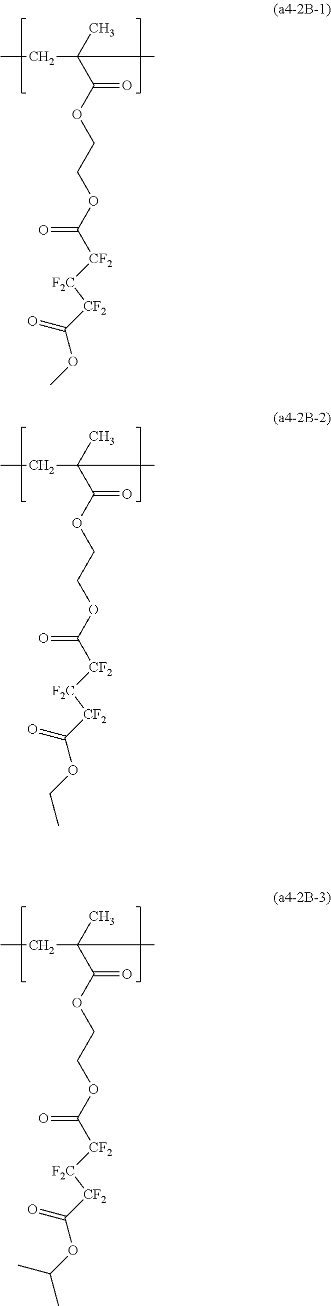

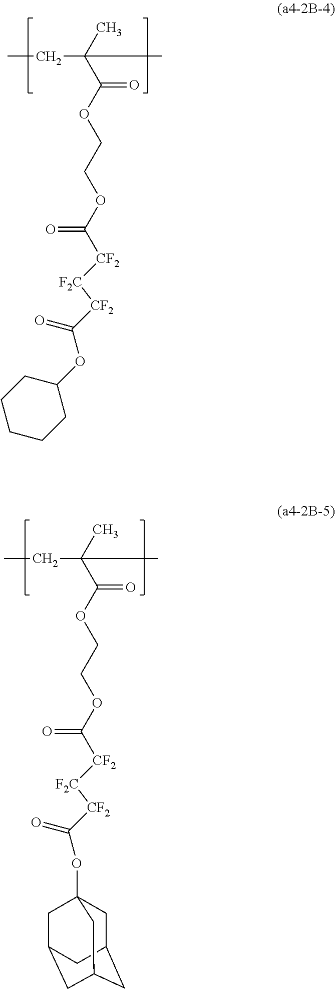

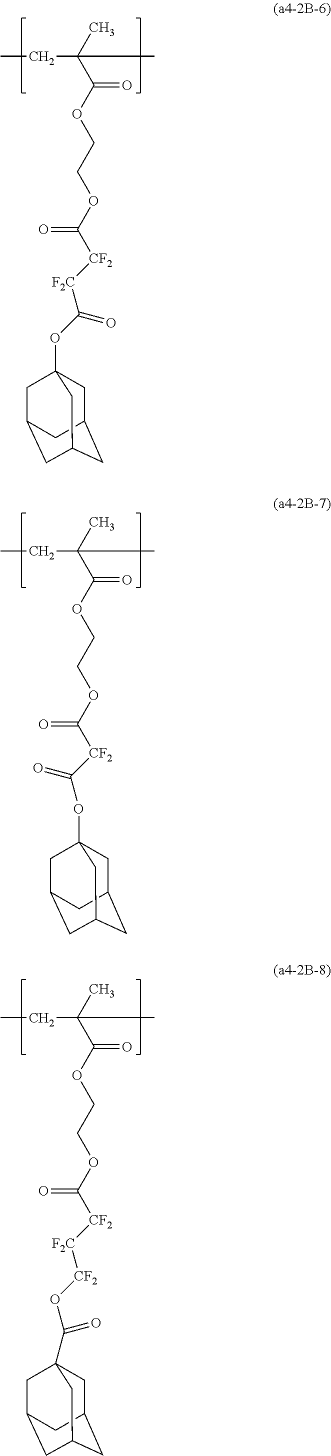

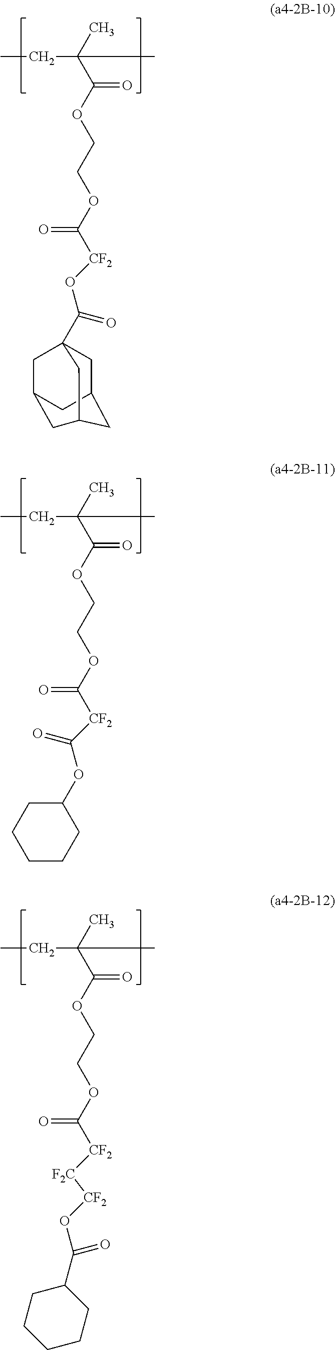

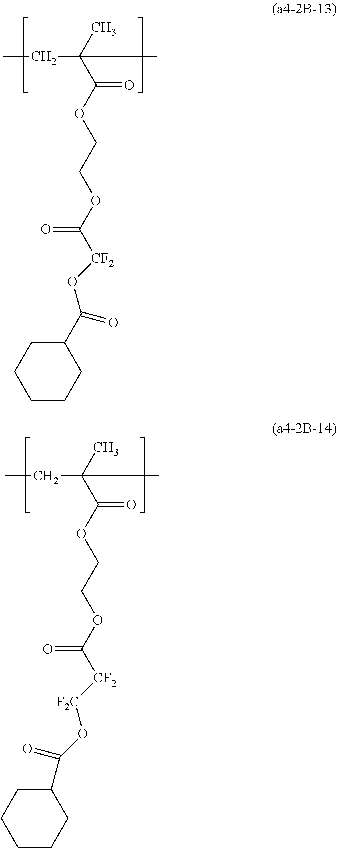

Examples of the structural unit (a4) also include a structural unit represented by the formula (a4-3).

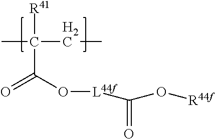

[In the formula (a4-3),
R⁴¹ represents a hydrogen atom or a methyl group.

L^{44f} represents an aliphatic hydrocarbon group having 1 to 20 carbon atoms and optionally having a fluorine atom, and —CH₂— included in the aliphatic hydrocarbon group may be substituted with —O— or —CO—.

R^{44f} represents an aliphatic hydrocarbon group having 1 to 20 carbon atoms and optionally having a fluorine atom, and —CH₂— included in the aliphatic hydrocarbon group may be substituted with —O— or —CO—.

Provided that at least one of L^{44f} and R^{44f} has a fluorine atom, and the upper limit of the total number of carbon atoms of L^{44f} and R^{44f} is 21]

Examples of the aliphatic hydrocarbon group in L^{44f} and R^{44f} include a linear or branched chain aliphatic hydrocarbon group and a monocyclic or polycyclic alicyclic hydrocarbon group, and groups formed by combining these groups. Examples of the aliphatic hydrocarbon group in L^{44f} and R^{44f} include groups that are the same as aliphatic hydrocarbon groups as described as for R⁴² in the formula (a4) as long as the upper limit of the number of carbon atoms of L^{44f} and R^{44f} is acceptable.

L^{44f} is preferably an alkanediyl group having 1 to 14 carbon atoms (—CH₂— included in the alkanediyl group may be substituted with —O— or —CO—), and more preferably a group represented by —(CH₂)ⱼ₁—, —(CH₂)ⱼ₂—O—(CH₂)ⱼ₃— or —(CH₂)ⱼ₄—CO—O—(CH₂)ⱼ₅— (j1 to j5 each independently represent an integer of 1 to 6). In addition, it is preferably an alkanediyl group having 1 to 4 carbon atoms (one —CH₂— included in the alkanediyl group may be substituted with —O—, and one —CH₂—CH₂— included in the alkanediyl group may be substituted with —CO—O— or —O—CO—).

R^{44f} is preferably an aliphatic hydrocarbon group having 1 to 10 carbon atoms and having a fluorine atom, more preferably an alkyl group having 1 to 10 carbon atoms and having a fluorine atom, an alicyclic hydrocarbon group having 3 to 10 carbon atoms and having a fluorine atom, or groups obtained by combining these groups, still more preferably an alkyl group having 1 to 10 carbon atoms and having a fluorine atom, and yet more preferably an alkyl group having 1 to 6 carbon atoms and having a fluorine atom.

Examples of the structural unit represented by the formula (a4-3) include the following structural units, and structural units in which the methyl group corresponding to R⁴¹ in the structural unit (a4-3) is substituted with a hydrogen atom in the structural units represented by the following formulas.

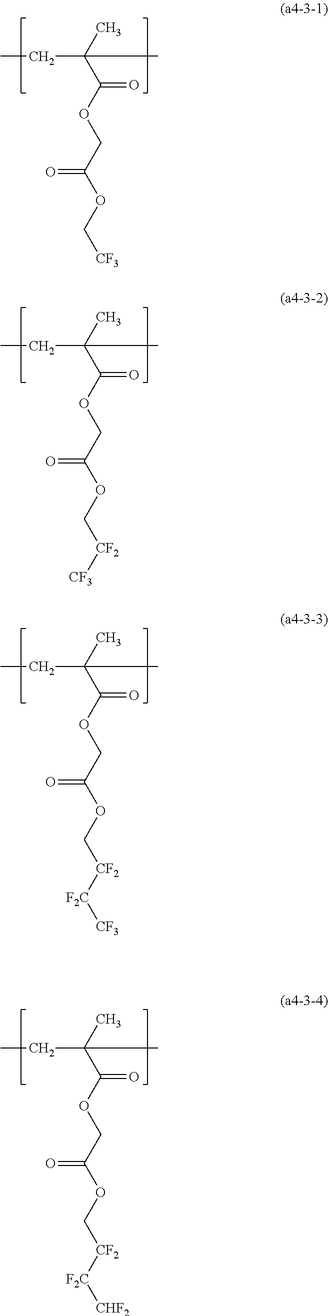

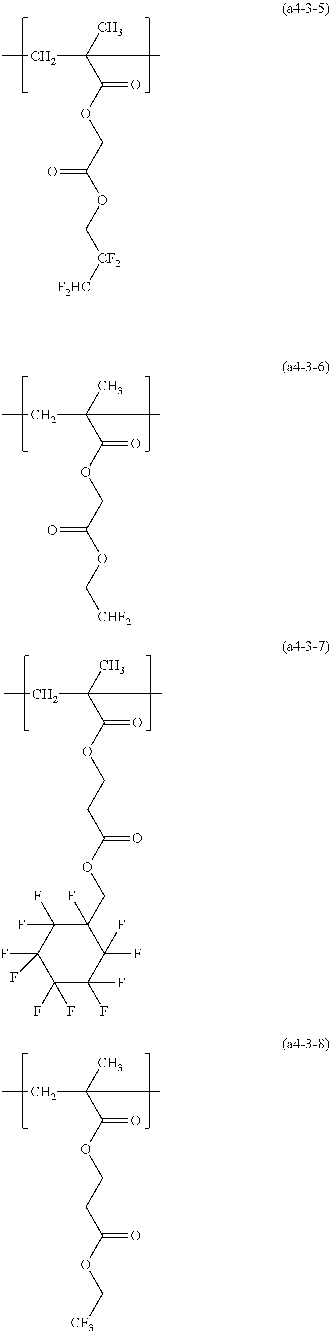

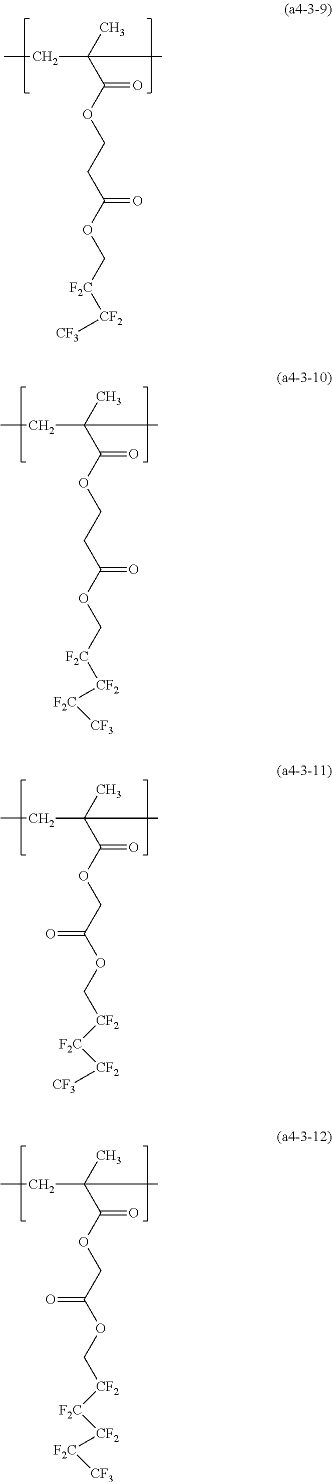

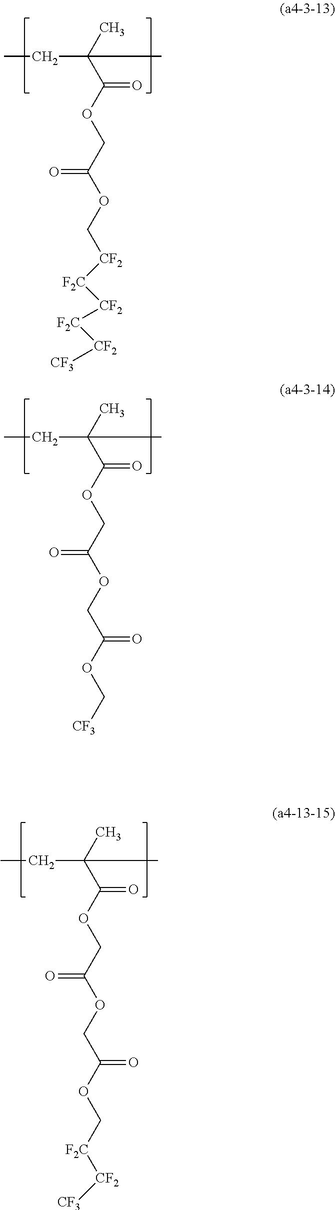

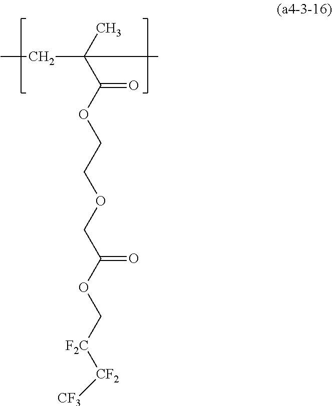

Examples of the structural unit (a4) also include a structural unit represented by the formula (a4-A).

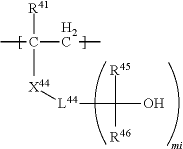

[In the formula (a4-A),
R⁴⁴ represents a hydrogen atom, a halogen atom, or an alkyl group having 1 to 6 carbon atoms and optionally having a halogen atom.

X⁴⁴ represents a single bond or *—CO—O—. * represents a binding site to carbon atoms to which R⁴⁴ is bonded.

L⁴⁴ represents an aliphatic hydrocarbon group having 1 to 28 carbon atoms and optionally having a substituent, and —CH₂— included in the aliphatic hydrocarbon group may be substituted with —O—, —S—, —SO₂—, —SO—, or —CO—.

R⁴⁵ represents an alkyl fluoride group having 1 to 8 carbon atoms.

R⁴⁶ represents a hydrogen atom or an alkyl fluoride group having 1 to 6 carbon atoms, the alkyl fluoride group may be bonded to L⁴⁴ to form an alicyclic hydrocarbon group having 3 to 12 carbon atoms, and —CH₂— included in the alicyclic hydrocarbon group may be substituted with —O—.

mi represents an integer of 1 to 4. When mi is 2 or more, a plurality of groups in parentheses may be the same as or different from each other]

Examples of the halogen atom and the alkyl group optionally having a halogen atom in R⁴⁴ include those that are the same as described as for R^{a01}, R^{a4}, and R^{a5} of the formula (a1-0), formula (a1-1), and formula (a1-2).

R⁴⁴ is preferably a hydrogen atom or an alkyl group having 1 to 4 carbon atoms, more preferably a hydrogen atom or an alkyl group having 1 to 3 carbon atoms, still more preferably a hydrogen atom, a methyl group or an ethyl group, and yet more preferably a hydrogen atom or a methyl group.

Examples of the aliphatic hydrocarbon group in L⁴⁴ include chain hydrocarbon groups, and monocyclic or polycyclic (including a spiro ring, a fused ring or a bridged ring) alicyclic hydrocarbon groups, and the aliphatic hydrocarbon group may be groups obtained by combining these two or more groups (for example, a hydrocarbon group formed from an alicyclic hydrocarbon group and a chain hydrocarbon group).

Examples of the chain hydrocarbon group include divalent to pentavalent chain hydrocarbon groups such as an alkanediyl group, an alkanetriyl group, an alkanetetrayl group, and an alkanepentayl group. Specific examples include those that are the same as described herein as long as the upper limit of the number of carbon atoms is acceptable. The number of carbon atoms of the chain hydrocarbon group is preferably 1 to 18, more preferably 1 to 12, still more preferably 1 to 10, yet more preferably 1 to 9, further preferably 1 to 8, still further preferably 1 to 6, yet still further preferably 1 to 5, and particularly preferably 1 to 4.

Examples of the monocyclic or polycyclic alicyclic hydrocarbon group include divalent to pentavalent alicyclic hydrocarbon groups such as a cycloalkanediyl group, a cycloalkanetriyl group, a cycloalkanetetrayl group, and a cycloalkanepentayl group. Specific examples include those that are the same as described herein as long as the upper limit of the number of carbon atoms is acceptable.

The number of carbon atoms of the alicyclic hydrocarbon group is preferably 3 to 20, more preferably 3 to 18, still more preferably 3 to 16, and yet more preferably 3 to 12.

Examples of the group obtained by combining two or more groups include a group obtained by combining an alicyclic hydrocarbon group with a chain hydrocarbon group. In combination, two or more of alicyclic hydrocarbon groups and chain hydrocarbon groups may be respectively combined. In addition, any group may also be bonded to X⁴⁴.

Examples of the group obtained by combining an alicyclic hydrocarbon group with a chain hydrocarbon group include a group in which an alicyclic hydrocarbon group is bonded to a chain hydrocarbon group (for example, a *-chain hydrocarbon group-alicyclic hydrocarbon group- and the like), a group in which a chain hydrocarbon group is bonded to an alicyclic hydrocarbon group (for example, an *-alicyclic hydrocarbon group-chain hydrocarbon group- and the like), a group in which an alicyclic hydrocarbon group and a chain hydrocarbon group are bonded to a chain hydrocarbon group (for example, a *-chain hydrocarbon group-alicyclic hydrocarbon group-chain hydrocarbon group- and the like), a group in which a chain hydrocarbon group and an alicyclic hydrocarbon group are bonded to an alicyclic hydrocarbon group (for example, an *-alicyclic hydrocarbon group-chain hydrocarbon group-alicyclic hydrocarbon group- and the like), a group in which an alicyclic hydrocarbon group, a chain hydrocarbon group, and an alicyclic hydrocarbon group are bonded to a chain hydrocarbon group (for example, a *-chain hydrocarbon group-alicyclic hydrocarbon group-chain hydrocarbon group-alicyclic hydrocarbon group- and the like), a group in which a chain hydrocarbon group, an alicyclic hydrocarbon group, and a chain hydrocarbon group are bonded to an alicyclic hydrocarbon group (for example, an *-alicyclic hydrocarbon group-chain hydrocarbon group-alicyclic hydrocarbon group-chain hydrocarbon group- and the like).

* represents a binding site to X⁴⁴.

In addition, examples thereof include groups in which one or more hydrogen atoms of the above-described groups are substituted with a binding site.

—CH₂— included in the aliphatic hydrocarbon group having 1 to 28 carbon atoms as for L⁴⁴ may be substituted with —O—, —S—, —SO—, —SO₂—, or —CO—.

When —CH₂— included in the aliphatic hydrocarbon group having 1 to 28 carbon atoms as for L⁴⁴ is substituted with —O—, —S—, —SO—, —SO₂—, or —CO—, the number of carbon atoms before substitution is defined as the number of carbon atoms of the aliphatic hydrocarbon group.

Examples of the group in which —CH₂— included in an aliphatic hydrocarbon group is substituted with —O—, —S—, —SO₂—, —SO—, or —CO— include a hydroxy group, a carboxy group, a thiol group, an alkoxy group, an alkylthio group, an alkoxycarbonyl group, an alkylsulfonyl group, an alkylcarbonyl group, an alkylcarbonyloxy group, an alkoxycarbonyloxy group, an oxy group, a carbonyl group, a thio group, a sulfonyl group, an alkanediyloxy group, an alkanediyloxycarbonyl group, an alkanediylcarbonyl group, an alkanediylcarbonyloxy group, an alkanediylsulfonyl group, an alkanediylthio group, a cycloalkoxy group, a cycloalkylalkoxy group, and a group obtained by combining two or more of these groups. Examples of these substituted groups include those that are the same as described in the present specification as long as the upper limit of the number of carbon atoms permits. The number of carbon atoms of these substituted groups is, for example, 1 to 17, preferably 1 to 11, more preferably 1 to 6, still more preferably 1 to 4, and yet more preferably 1 to 3 in the case of an alkoxy group or the like, 2 to 17, preferably 2 to 11, more preferably 2 to 6, still more preferably 2 to 4, and yet more preferably 2 or 3 in the case of an alkoxycarbonyl group or the like, 2 to 18, preferably 2 to 12, more preferably 2 to 6, still more preferably 2 to 4, and yet more preferably 2 or 3 in the case of an alkylcarbonyl group or the like, and is 2 to 16, preferably 2 to 10, more preferably 2 to 6, still more preferably 2 to 4, and yet more preferably 2 or 3 in the case of an alkoxycarbonyloxy group or the like.

In addition, examples thereof include groups in which one or more hydrogen atoms of the above-described groups are substituted with a binding site.

Examples of the group in which —CH₂— included in the alicyclic hydrocarbon group is substituted with —O—, —S—, —CO—, or —SO₂— include those that are the same as described in the present specification as long as the upper limit of the number of carbon atoms is acceptable.

The aliphatic hydrocarbon group in L⁴⁴ may have one or a plurality of substituents. Examples of the substituent include a halogen atom, a haloalkyl group having 1 to 4 carbon atoms, an alkyl group having 1 to 12 carbon atoms, an alicyclic hydrocarbon group having 3 to 18 carbon atoms or an aromatic hydrocarbon group having 6 to 18 carbon atoms (—CH₂— included in the alkyl group or the alicyclic hydrocarbon group may be substituted by —O— or —CO—).

Examples of the halogen atom include a fluorine atom, a chlorine atom, a bromine atom, and an iodine atom.

Examples of the haloalkyl group having 1 to 4 carbon atoms include an alkyl fluoride group having 1 to 4 carbon atoms, an alkyl chloride group having 1 to 4 carbon atoms, an alkyl bromide group having 1 to 4 carbon atoms, and an alkyl iodide group having 1 to 4 carbon atoms. Examples of the haloalkyl group include a perfluoroalkyl group having 1 to 4 carbon atoms (trifluoromethyl group, pentafluoroethyl group, heptafluoropropyl group, nonafluorobutyl group, and the like), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, and an iodomethyl group. The number of carbon atoms of the haloalkyl group is preferably 1 to 3, and more preferably 1 or 2.

Examples of the alkyl group having 1 to 12 carbon atoms include a methyl group, an ethyl group, a propyl group, a butyl group, a pentyl group, a hexyl group, a heptyl group, an octyl group, a nonyl group, a decyl group, an undecyl group, and a dodecyl group. The number of carbon atoms of the alkyl group is preferably 1 to 9, more preferably 1 to 6, still more preferably 1 to 4, and yet more preferably 1 to 3.

Examples of the alicyclic hydrocarbon group having 3 to 18 carbon atoms include monocyclic cycloalkyl groups such as a cyclopropyl group, a cyclobutyl group, a cyclopentyl group, a cyclohexyl group, a cycloheptyl group, a cyclooctyl group, and a cyclodecyl group; and polycyclic cycloalkyl groups such as a decahydronaphthyl group, an adamantyl group, and a norbornyl group. The number of carbon atoms of the alicyclic hydrocarbon group is preferably 3 to 16, more preferably 3 to 12, and still more preferably 3 to 10.

Examples of the aromatic hydrocarbon group having 6 to 18 carbon atoms include a phenyl group, a naphthyl group, a biphenyl group, an anthryl group, a phenanthryl group, and a binaphthyl group. The number of carbon atoms of the aromatic hydrocarbon group is preferably 6 to 14, more preferably 6 to 12, and still more preferably 6 to 10.

When —CH₂— included in the alkyl group is substituted with —O— or —CO—, the number of carbon atoms before substitution is defined as the total number of the alkyl group. Examples of the substituted group include a hydroxy group (a group in which —CH₂— included in the methyl group is substituted with —O—), a carboxy group (a group in which —CH₂—CH₂— included in the ethyl group is substituted with —O—CO—), an alkoxy group (a group in which —CH₂— at any position included in the alkyl group is substituted with —O—), an alkoxycarbonyl group (a group in which —CH₂—CH₂— at any position included in the alkyl group is substituted with —O—CO—), an alkylcarbonyl group (a group in which —CH₂— at any position included in the alkyl group is substituted with —CO—), alkylcarbonyloxy group (a group in which —CH₂—CH₂— at any position included in the alkyl group is substituted with —CO—O—), and an alkoxycarbonyloxy group (a group in which —CH₂—CH₂—CH₂— at any position included in the alkyl group is substituted with —O—CO—O—). Examples of these substituted groups include those that are the same as described in the present specification as long as the upper limit of the number of carbon atoms permits.

The number of carbon atoms of the alkoxy group is preferably 1 to 9, more preferably 1 to 6, still more preferably 1 to 4, and yet more preferably 1 to 3. The number of carbon atoms of the alkoxycarbonyl group is preferably 2 to 9, more preferably 2 to 6, still more preferably 2 to 4, and yet more preferably 2 or 3. The number of carbon atoms of the alkylcarbonyl group is preferably 2 to 10, more preferably 2 to 6, still more preferably 2 to 4, and yet more preferably 2 or 3. The number of carbon atoms of the alkylcarbonyloxy group is preferably 2 to 9, more preferably 2 to 6, still more preferably 2 to 4, and yet more preferably 2 or 3.

Examples of the group in which —CH₂— included in the alicyclic hydrocarbon group is substituted with —O— or —CO— include those that are the same as group described herein as long as the upper limit of the number of carbon atoms is acceptable.

The substituent which may be possessed by the aliphatic hydrocarbon group in L⁴⁴ is preferably a halogen atom, a haloalkyl group having 1 to 3 carbon atoms, an alkyl group having 1 to 6 carbon atoms, an alicyclic hydrocarbon group having 3 to 10 carbon atoms or an aromatic hydrocarbon group having 6 to 10 carbon atoms (—CH₂— included in the alkyl group or the alicyclic hydrocarbon group may be substituted with —O— or —CO—), more preferably a fluorine atom, an iodine atom, a perfluoroalkyl group having 1 to 3 carbon atoms or an alkyl group having 1 to 4 carbon atoms (—CH₂— included in the alkyl group may be substituted with —O— or —CO—), and still more preferably a fluorine atom, an iodine atom, a trifluoromethyl group, a methyl group, a hydroxy group, or a methoxy group.

When an alicyclic hydrocarbon group is not formed with a fluorinated alkyl group as for R⁴⁶, L⁴⁴ is preferably a chain hydrocarbon group having 1 to 12 carbon atoms and optionally having a substituent (—CH₂— included in the chain hydrocarbon group may be substituted with —O— or —CO—), an alicyclic hydrocarbon group having 3 to 18 carbon atoms and optionally having a substituent (—CH₂— included in the alicyclic hydrocarbon group may be substituted with —O— or —CO—) or a group obtained by combining a chain hydrocarbon group having 1 to 6 carbon atoms and optionally having a substituent with an alicyclic hydrocarbon group having 3 to 18 carbon atoms and optionally having a substituent (—CH₂— included in the chain hydrocarbon group may be substituted with —O— or —CO—, and —CH₂— included in the alicyclic hydrocarbon group may be substituted with —O— or —CO—), more preferably a chain hydrocarbon group having 1 to 8 carbon atoms and optionally having a substituent (—CH₂— included in the chain hydrocarbon group may be substituted with —O— or —CO—), an alicyclic hydrocarbon group having 3 to 12 carbon atoms and optionally having a substituent or a group obtained by combining a chain hydrocarbon group having 1 to 6 carbon atoms and optionally having a substituent with an alicyclic hydrocarbon group having 3 to 18 carbon atoms and optionally having a substituent (—CH₂— included in the chain hydrocarbon group may be substituted with —O— or —CO—), and still more preferably a chain hydrocarbon group having 1 to 8 carbon atoms and optionally having a substituent (—CH₂— included in the chain hydrocarbon group may be substituted with —O— or —CO—), an alicyclic hydrocarbon group having 5 to 12 carbon atoms and optionally having a substituent, a group obtained by combining a chain hydrocarbon group having 1 to 6 carbon atoms with an alicyclic hydrocarbon group having 5 to 12 carbon atoms and optionally having a substituent (—CH₂— included in the chain hydrocarbon group may be substituted with —O— or —CO—) or a group obtained by combining an alicyclic hydrocarbon group having 5 to 12 carbon atoms and optionally having a substituent, and a chain hydrocarbon group having 1 to 6 carbon atoms and an alicyclic hydrocarbon group having 5 to 12 carbon atoms and optionally having a substituent (—CH₂— included in the chain hydrocarbon group may be substituted with —O— or —CO—).

Examples of the fluorinated alkyl group having 1 to 8 carbon atoms in R⁴⁵ include the same groups as those exemplified for R^{43fB} of the formula (a4-2B). The number of carbon atoms of the fluorinated alkyl group is preferably 1 to 6, more preferably 1 to 5, still more preferably 1 to 4, and yet more preferably 1 to 3.

Examples of the fluorinated alkyl group having 1 to 6 carbon atoms in R⁴⁶ similarly include the alkyl fluoride group having 1 to 6 carbon atoms described as examples of the alkyl fluoride group as for R⁴⁵ as long as the upper limit of the number of carbon atoms is acceptable.

When R⁴⁶ is a fluorinated alkyl group having 1 to 6 carbon atoms, R⁴⁵ is preferably an alkyl fluoride group having 1 to 6 carbon atoms, more preferably an alkyl fluoride group having 1 to 3 carbon atoms, still more preferably a trifluoromethyl group, a perfluoroethyl group or a perfluoropropyl group, and yet more preferably a trifluoromethyl group.

When R⁴⁶ is a hydrogen atom, R⁴⁵ is preferably an alkyl fluoride group having 1 to 6 carbon atoms, more preferably an alkyl fluoride group having 1 to 5 carbon atoms, and still more preferably an alkyl fluoride group having 3 to 5 carbon atoms.

When R⁴⁶ is bonded with L⁴⁴ to form an alicyclic hydrocarbon group having 3 to 12 carbon atoms, examples of the group formed by bonding R⁴⁶ with L⁴⁴ include groups represented by the following formulas. Among the following formulas, * and ** represent a binding site, one of two ** represents a binding site to a hydroxy group, and the other represents a binding site to R⁴⁵, L^{44A} is part of the hydrocarbon group as for L⁴⁴ and represents a single bond or an alkanediyl group having 1 to 4 carbon atoms. The number of carbon atoms of the alicyclic hydrocarbon group formed by bonding R⁴⁶ with L⁴⁴ is preferably 3 to 10, more preferably 3 to 8, and still more preferably 3 to 6.

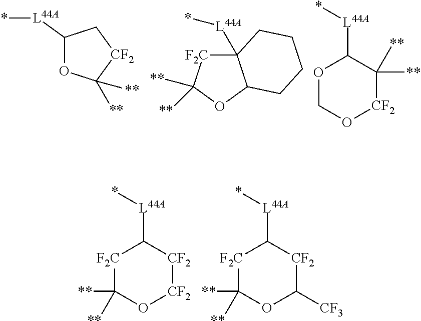

R⁴⁶ is preferably a hydrogen atom or an alkyl fluoride group having 1 to 3 carbon atoms.

mi is preferably an integer of 1 to 3, more preferably 1 or 2, and still more preferably 1.

Examples of the structural unit (a4-A) include the following structural units. It is also possible to exemplify, as specific examples of the structural unit (a4-A), a structural unit in which the methyl group corresponding to R⁴⁴ is substituted with a hydrogen atom, a halogen atom, a haloalkyl group, or an alkyl group other than the methyl group, and a structural unit in which the hydrogen atom corresponding to R⁴⁴ is substituted with a halogen atom, a haloalkyl group or an alkyl group in the following structural units.

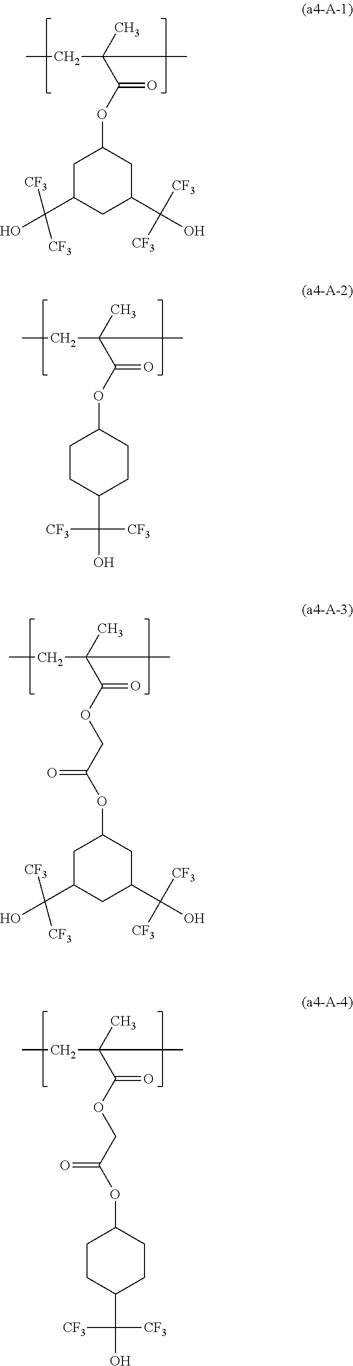

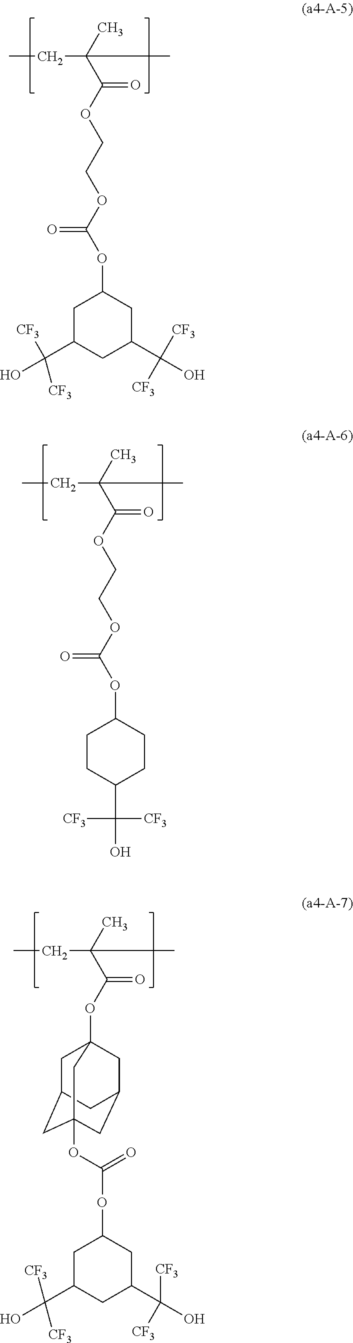

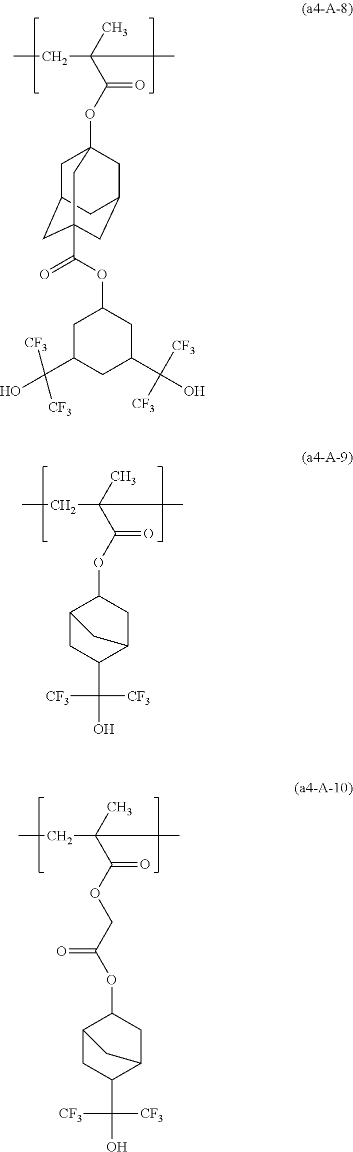

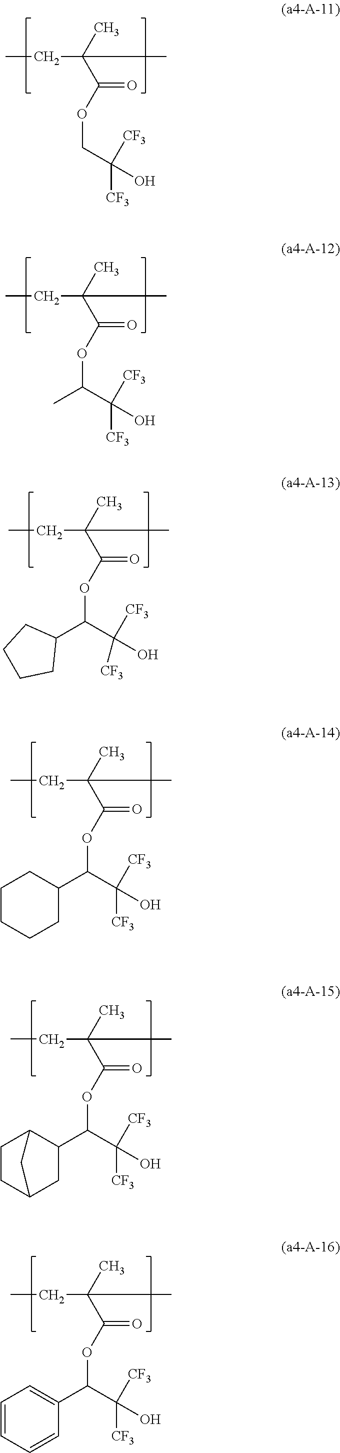

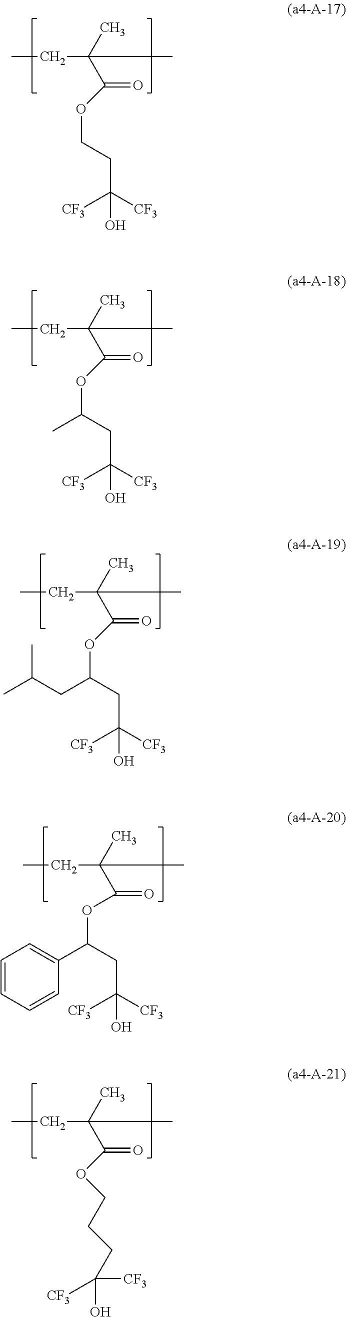

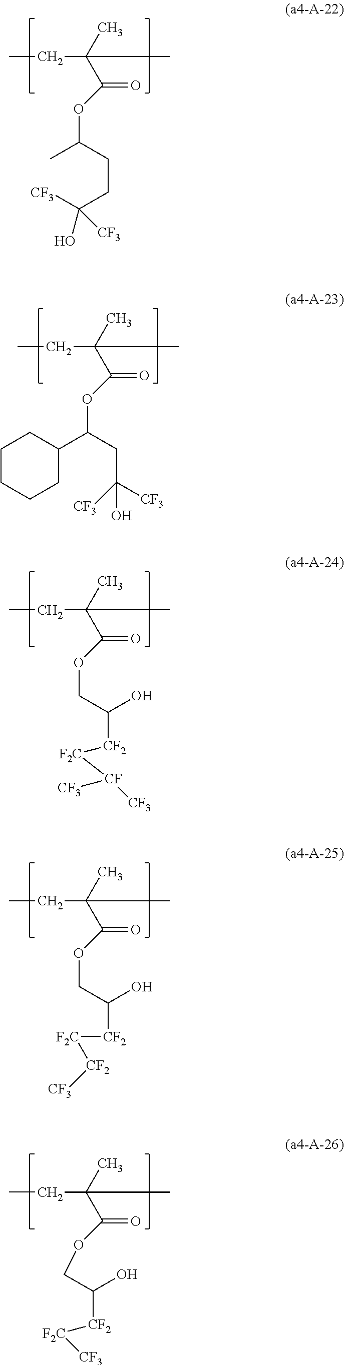

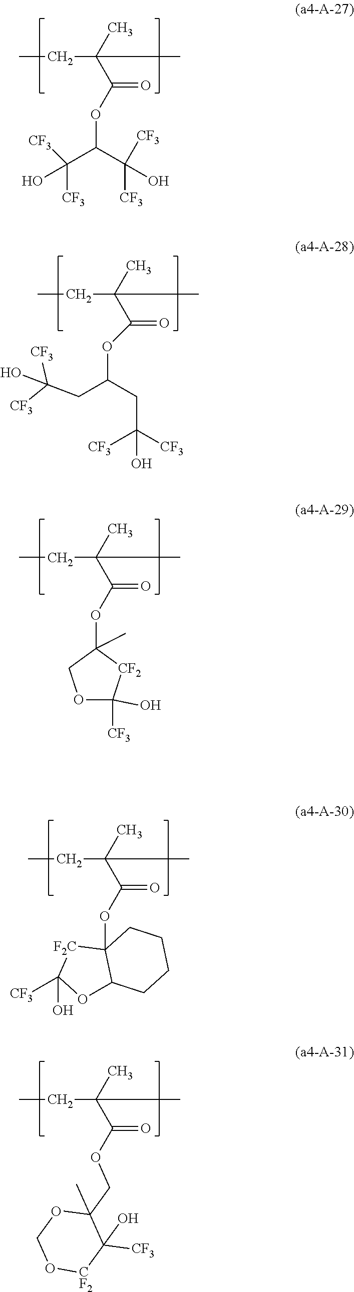

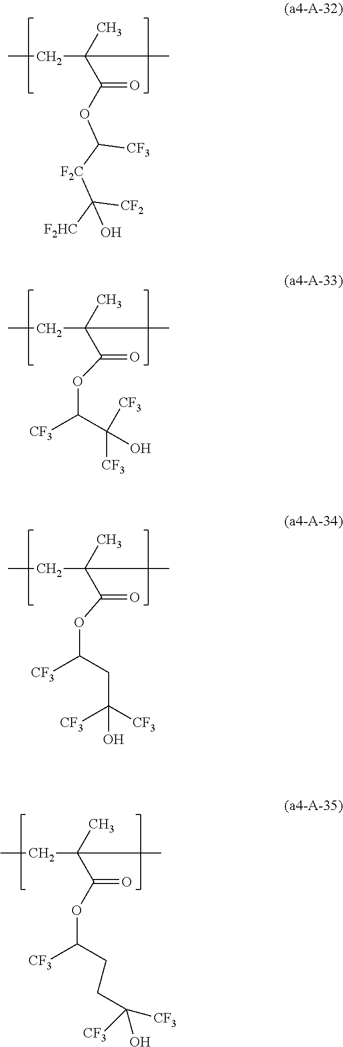

When the resin (A) or the like includes the structural unit (a4), the content is preferably 1 to 20 mol %, more preferably 2 to 15 mol %, and still more preferably 3 to 10 mol %, based on all structural units of the resin (A) or the like.

<Structural Unit (a5)>

Examples of a non-leaving hydrocarbon group possessed by the structural unit (a5) include groups having a linear, branched, or cyclic hydrocarbon group. Among these, the structural unit (a5) is preferably a group having an alicyclic hydrocarbon group.

The structural unit (a5) includes, for example, a structural unit represented by a formula (a5-1).

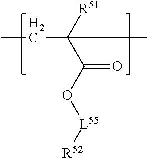

[In the formula (a5-1),
R⁵¹ represents a hydrogen atom or a methyl group.

R⁵² represents an alicyclic hydrocarbon group having 3 to 18 carbon atoms, and a hydrogen atom included in the alicyclic hydrocarbon group may be substituted with an aliphatic hydrocarbon group having 1 to 8 carbon atoms.

L⁵⁵ represents a single bond or a divalent saturated hydrocarbon group having 1 to 18 carbon atoms, and —CH₂— included in the saturated hydrocarbon group may be substituted with —O— or —CO—.

The alicyclic hydrocarbon group in R⁵² may be either monocyclic or polycyclic. The monocyclic alicyclic hydrocarbon group includes, for example, a cyclopropyl group, a cyclobutyl group, a cyclopentyl group, and a cyclohexyl group. The polycyclic alicyclic hydrocarbon group includes, for example, an adamantyl group and a norbornyl group.

The aliphatic hydrocarbon group having 1 to 8 carbon atoms includes, for example, alkyl groups such as a methyl group, an ethyl group, a propyl group, an isopropyl group, a butyl group, a sec-butyl group, a tert-butyl group, a pentyl group, a hexyl group, an octyl group, and a 2-ethylhexyl group.

Examples of the alicyclic hydrocarbon group having a substituent include a 3-methyladamantyl group.

R⁵² is preferably an unsubstituted alicyclic hydrocarbon group having 3 to 18 carbon atoms, and more preferably an adamantyl group, a norbornyl group, or a cyclohexyl group.

Examples of the divalent saturated hydrocarbon group in L⁵⁵ include a divalent chain saturated hydrocarbon group and a divalent alicyclic saturated hydrocarbon group, and a divalent chain saturated hydrocarbon group is preferable.

The divalent chain saturated hydrocarbon group includes, for example, alkanediyl groups such as a methylene group, an ethylene group, a propanediyl group, a butanediyl group, and a pentanediyl group.

The divalent alicyclic saturated hydrocarbon group may be either monocyclic or polycyclic. Examples of the monocyclic alicyclic saturated hydrocarbon group include cycloalkanediyl groups such as a cyclopentanediyl group and a cyclohexanediyl group. Examples of the polycyclic divalent alicyclic saturated hydrocarbon group include an adamantanediyl group and a norbornanediyl group.

The group in which —CH₂— included in the divalent saturated hydrocarbon group represented by L⁵⁵ is substituted with —O— or —CO— includes, for example, groups represented by a formula (L1-1) to a formula (L1-4). In the following formulas, * and ** each represent a binding site, and * represents a binding site to an oxygen atom.

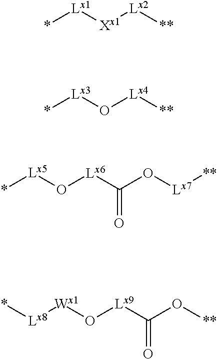

[In the formula (L1-1),
X^{x1} represents *—O—CO— or *—CO—O— (* represents a binding site to L^{x1}).

L^{x1} represents a divalent aliphatic saturated hydrocarbon group having 1 to 16 carbon atoms.

L^{x2} represents a single bond or a divalent aliphatic saturated hydrocarbon group having 1 to 15 carbon atoms.

The total number of carbon atoms of L^{x1} and L^{x2} is 16 or less.

In the formula (L1-2),

L^{x3} represents a divalent aliphatic saturated hydrocarbon group having 1 to 17 carbon atoms.

L^{x4} represents a single bond or a divalent aliphatic saturated hydrocarbon group having 1 to 16 carbon atoms.

The total number of carbon atoms of L^{x3} and L^{x4} is 17 or less.

In the formula (L1-3),

L^{x5} represents a divalent aliphatic saturated hydrocarbon group having 1 to 15 carbon atoms.

L^{x6} and L^{x7} each independently represent a single bond or a divalent aliphatic saturated hydrocarbon group having 1 to 14 carbon atoms.

The total number of carbon atoms of L^{x5}, L^{x6}, and L^{x7} is 15 or less.

In the formula (L1-4),

L^{x8} and L^{x9} represent a single bond or a divalent aliphatic saturated hydrocarbon group having 1 to 12 carbon atoms.

W^{x1} represents a divalent alicyclic saturated hydrocarbon group having 3 to 15 carbon atoms.

The total number of carbon atoms of L^{x8}, L^{x9}, and W^{x1} is 15 or less.]

L^{x1} is preferably a divalent aliphatic saturated hydrocarbon group having 1 to 8 carbon atoms, and more preferably a methylene group or an ethylene group.

L^{x2} is preferably a single bond or a divalent aliphatic saturated hydrocarbon group having 1 to 8 carbon atoms, and more preferably a single bond.

L^{x3} is preferably a divalent aliphatic saturated hydrocarbon group having 1 to 8 carbon atoms.

L^{x4} is preferably a single bond or a divalent aliphatic saturated hydrocarbon group having 1 to 8 carbon atoms.

L^{x5} is preferably a divalent aliphatic saturated hydrocarbon group having 1 to 8 carbon atoms, and more preferably a methylene group or an ethylene group.

L^{x6} is preferably a single bond or a divalent aliphatic saturated hydrocarbon group having 1 to 8 carbon atoms, and more preferably a methylene group or an ethylene group.

L^{x7} is preferably a single bond or a divalent aliphatic saturated hydrocarbon group having 1 to 8 carbon atoms.

L^{x8} is preferably a single bond or a divalent aliphatic saturated hydrocarbon group having 1 to 8 carbon atoms, and more preferably a single bond or a methylene group.

L^{x9} is preferably a single bond or a divalent aliphatic saturated hydrocarbon group having 1 to 8 carbon atoms, and more preferably a single bond or a methylene group.

W^{x1} is preferably a divalent alicyclic saturated hydrocarbon group having 3 to 10 carbon atoms, and more preferably a cyclohexanediyl group or an adamantanediyl group.

The group represented by the formula (L1-1) includes, for example, the following divalent groups.

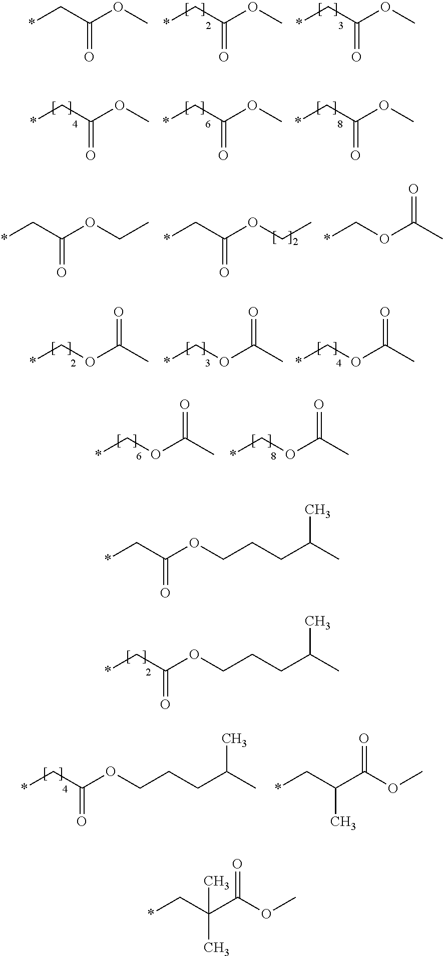

The group represented by the formula (L1-2) includes, for example, the following divalent groups.

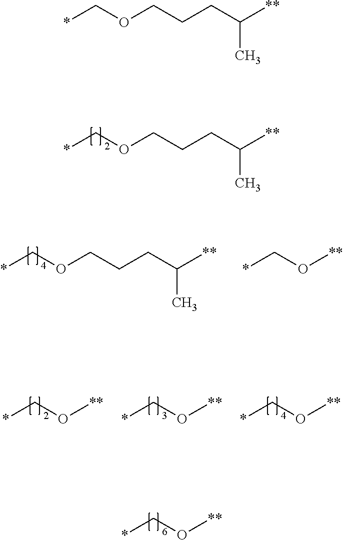

The group represented by the formula (L1-3) includes, for example, the following divalent groups.

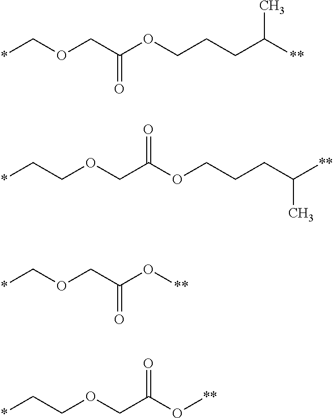

The group represented by the formula (L1-4) includes, for example, the following divalent groups.

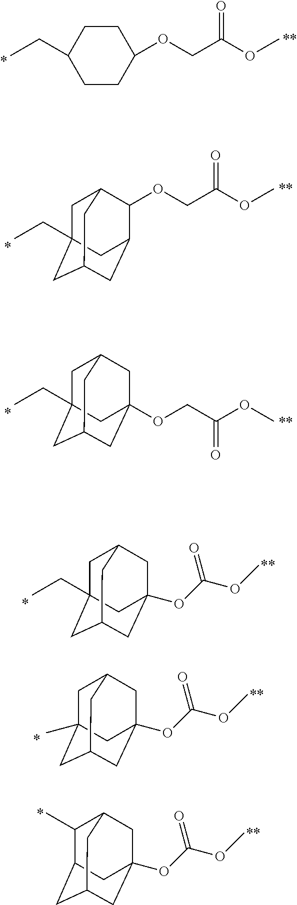

L⁵⁵ is preferably a single bond or a group represented by the formula (L1-1).

Examples of the structural unit (a5-1) include the following structural units and structural units in which a methyl group corresponding to R⁵¹ in the structural unit (a5-1) in the following structural units is substituted with a hydrogen atom.

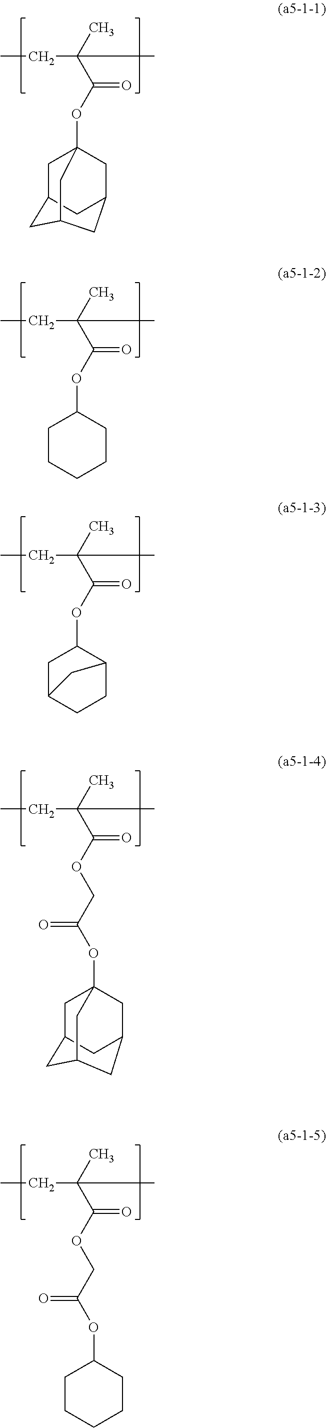

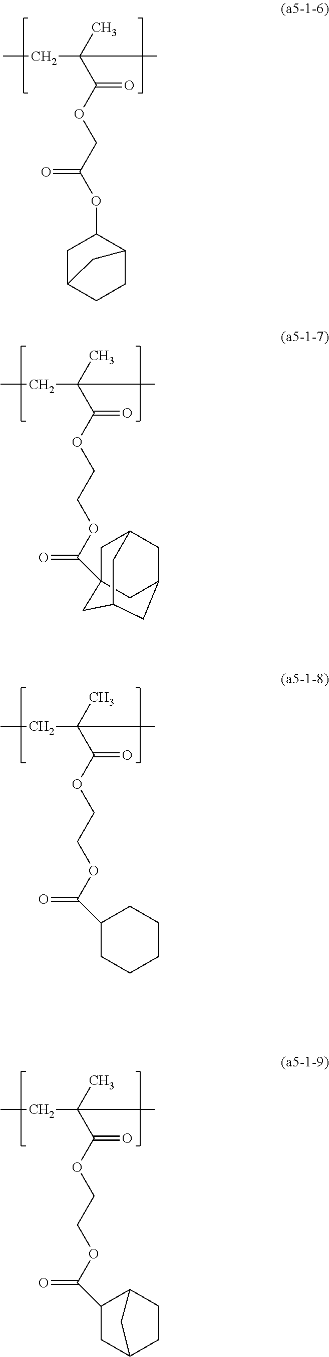

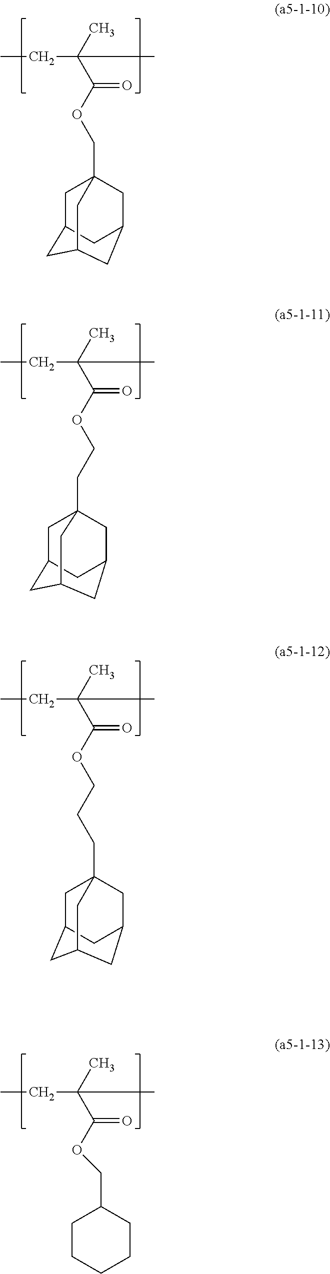

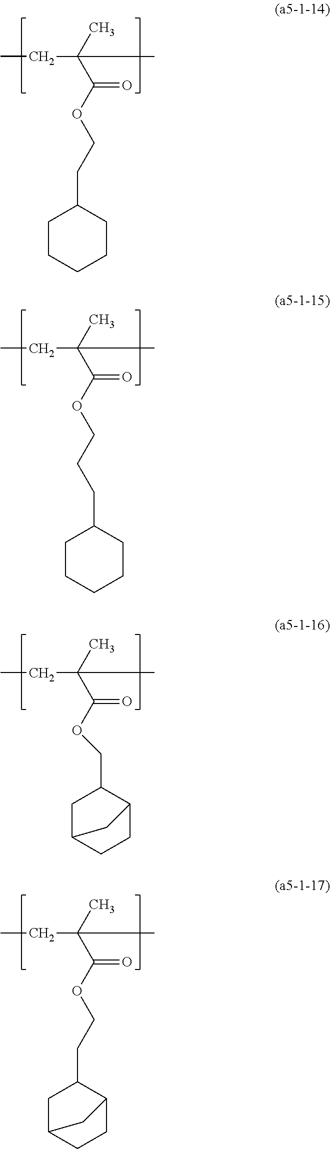

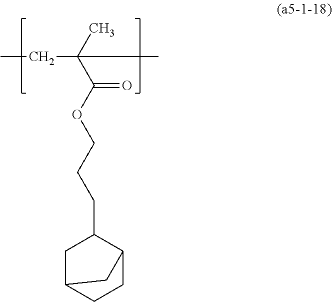

When the resin (A) or the like includes the structural unit (a5), the content is preferably 1 to 30 mol %, more preferably 2 to 20 mol %, and still more preferably 3 to 15 mol %, based on all structural units of the resin (A) or the like.

<Structural Unit (a6)>

The structural unit (a6) is a structural unit having an —SO₂— group, and it is preferable to have an —SO₂— group in a side chain.

The structural unit having an —SO₂— group may have a linear structure having an —SO₂— group, a branched structure having an —SO₂— group, or a cyclic structure (monocyclic and polycyclic structure) having an —SO₂— group. The structural unit is preferably a structural unit which has a cyclic structure having an —SO₂— group, and more preferably a structural unit having a cyclic structure (sultone ring) having —SO₂—O—.

Examples of the sultone ring include rings represented by the following formula (T¹-1), formula (T¹-2), formula (T¹-3), and formula (T¹-4). The binding site can be any position. The sultone ring may be monocyclic, and is preferably polycyclic. The polycyclic sultone ring means a bridged ring having —SO₂—O— as an atomic group constituting the ring, and examples thereof include rings represented by the formula (T¹-1) and the formula (T¹-2). The sultone ring may have, as the atomic group constituting the ring, a heteroatom, in addition to —SO₂—O—, like the ring represented by the formula (T¹-2). Examples of the heteroatom include an oxygen atom, a sulfur atom, or a nitrogen atom, and an oxygen atom is preferable.

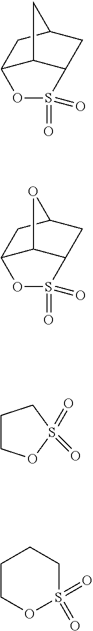 [SMILES: O=S1(=O)OC2CC3CC2C1C3 ; O=S1(=O)OC2CC3CC1C2O3 ; O=S1(=O)CCCO1 ; O=S1(=O)CCCCO1]

The sultone ring may have a substituent, and examples of the substituent include an alkyl group having 1 to 12 carbon atoms and optionally having a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms and an alkylcarbonyl group having 2 to 4 carbon atoms. Examples of these groups include the same groups as those exemplified herein.

Examples of the aryl group include a phenyl group, a naphthyl group, an anthryl group, a p-methylphenyl group, a p-tert-butylphenyl group, a p-adamantylphenyl group, a tolyl group, a xylyl group, a cumyl group, a mesityl group, a biphenyl group, a phenanthryl group, a 2,6-diethylphenyl group, and a 2-methyl-6-ethylphenyl group.

Examples of the aralkyl group include a benzyl group, a phenethyl group, a phenylpropyl group, a naphthylmethyl group, and a naphthylethyl group.

From the viewpoint that it is easy to produce a monomer from which the structural unit (a6) is derived, a sultone ring having no substituent is preferable.

The sultone ring is preferably a ring represented by the following formula (T1′).

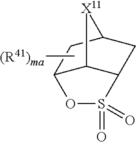

[In the formula (T1′),
X¹¹ represents an oxygen atom, a sulfur atom, or a methylene group,

R⁴¹ represents an alkyl group having 1 to 12 carbon atoms and optionally having a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, or an alkylcarbonyl group having 2 to 4 carbon atoms.

ma represents an integer of 0 to 9. When ma is 2 or more, a plurality of R⁴¹ may be the same or different.

the binding site may be any position.]

X¹¹ is preferably an oxygen atom or a methylene group, and more preferably a methylene group.

Examples of R⁴¹ include those that are the same as the substituent of the sultone ring, and an alkyl group having 1 to 12 carbon atoms and optionally having a halogen atom or a hydroxy group is preferable.

ma is preferably 0 or 1, and more preferably 0.

Examples of the ring represented by formula (T1′) include the following rings. The binding site may be any position. Particularly, the binding site is preferably the 1-position or the 3-position.

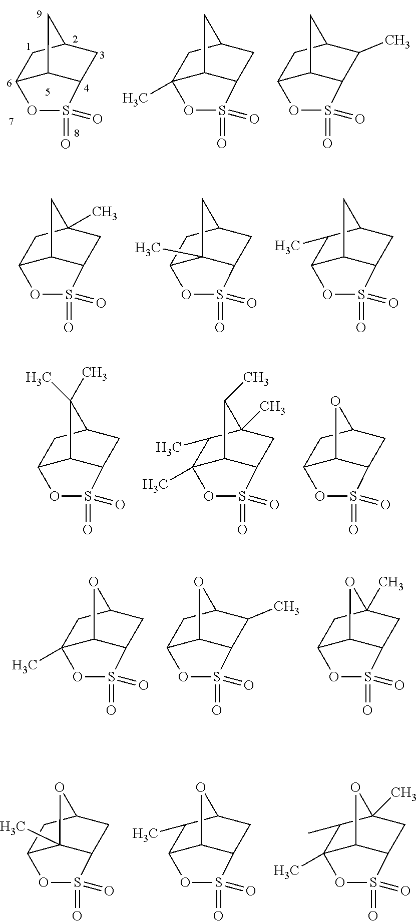

It is preferable that the structural unit having an —SO₂— group further has a group derived from a polymerizable group. Examples of the polymerizable group include a vinyl group, an acryloyl group, a methacryloyl group, an acryloyloxy group, a methacryloyloxy group, an acryloylamino group, a methacryloylamino group, an acryloylthio group, and a methacryloylthio group.

Particularly, the monomer from which the structural unit (a6) is derived is preferably a monomer having an ethylenically unsaturated bond, and more preferably a (meth)acrylic monomer.

The structural unit (a6) is preferably a structural unit represented by a formula (a6-0).

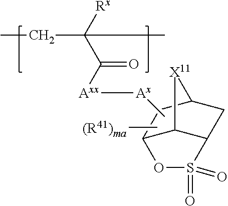

[In the formula (a6-0),

R^{x} represents an alkyl group having 1 to 6 carbon atoms and optionally having a halogen atom, a hydrogen atom, or a halogen atom.

A^{xx} represents an oxygen atom, —N(R^{c})—, or a sulfur atom.

A^{x} represents a single bond or a divalent saturated hydrocarbon group having 1 to 18 carbon atoms, and —CH₂— included in the saturated hydrocarbon group may be substituted with —O—, —CO—, or —N(R^{d})—.

X¹¹ represents an oxygen atom, a sulfur atom, or a methylene group,
R⁴¹ represents an alkyl group having 1 to 12 carbon atoms and optionally having a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, or an alkylcarbonyl group having 2 to 4 carbon atoms.

ma represents an integer of 0 to 9. When ma is 2 or more, a plurality of R⁴¹ may be the same or different.

R^{c} and R^{d} each independently represent a hydrogen atom or an alkyl group having 1 to 6 carbon atoms.]

Examples of R^{x} include the same groups as those exemplified for R^{a01} of the formula (a1-0).

Examples of the alkyl group as for R^{c} and R^{d} include a methyl group, an ethyl group, an n-propyl group, an isopropyl group, an n-butyl group, a sec-butyl group, a tert-butyl group, an n-pentyl group, and an n-hexyl group, and an alkyl group having 1 to 4 carbon atoms is preferable, and a methyl group or an ethyl group is more preferable.

R^{x} is preferably a hydrogen atom or an alkyl group having 1 to 4 carbon atoms, more preferably a hydrogen atom, a methyl group, or an ethyl group, and still more preferably a hydrogen atom or a methyl group.

Examples of the divalent saturated hydrocarbon group as for A^{x} include a linear alkanediyl group, a branched alkanediyl group, and a monocyclic or polycyclic divalent alicyclic saturated hydrocarbon group, and the divalent saturated hydrocarbon group may be those obtained by combining two or more of these groups.

Specific examples thereof include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, a heptadecane-1,17-diyl group, an ethane-1,1-diyl group, a propane-1,1-diyl group, and a propane-2,2-diyl group;
branched alkanediyl groups such as a butane-1,3-diyl group, a 2-methylpropane-1,3-diyl group, a 2-methylpropane-1,2-diyl group, a pentane-1,4-diyl group, and a 2-methylbutane-1,4-diyl group;
monocyclic divalent alicyclic saturated hydrocarbon groups that are cycloalkanediyl groups such as a cyclobutane-1,3-diyl group, a cyclopentane-1,3-diyl group, a cyclohexane-1,4-diyl group, and a cyclooctane-1,5-diyl group; and
polycyclic divalent alicyclic saturated hydrocarbon groups such as a norbornane-1,4-diyl group, a norbornane-2,5-diyl group, an adamantane-1,5-diyl group, and an adamantane-2,6-diyl group.

The binding site to the sultone ring as for A^{x} can be any position and is preferably the 1-position.

Examples of the structural unit (a6-0) include the following structural units.

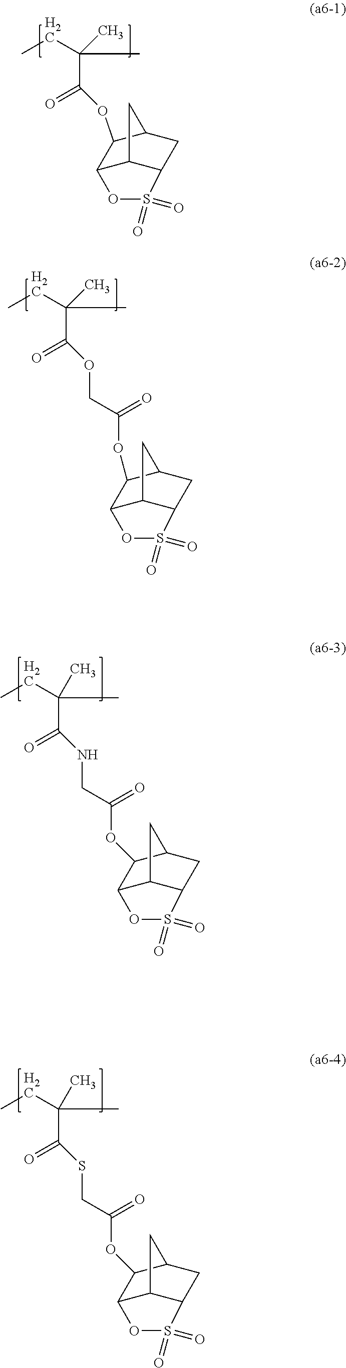

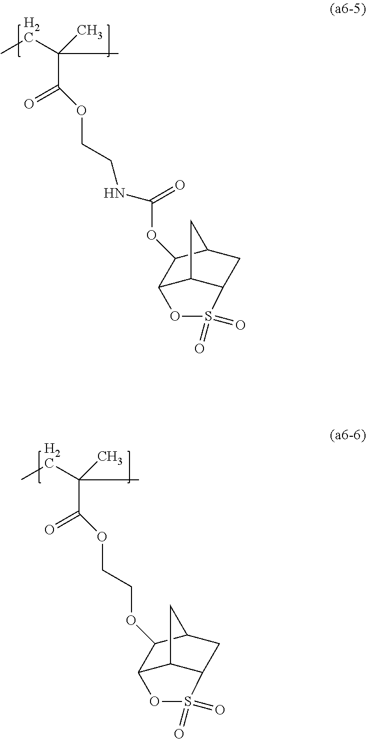

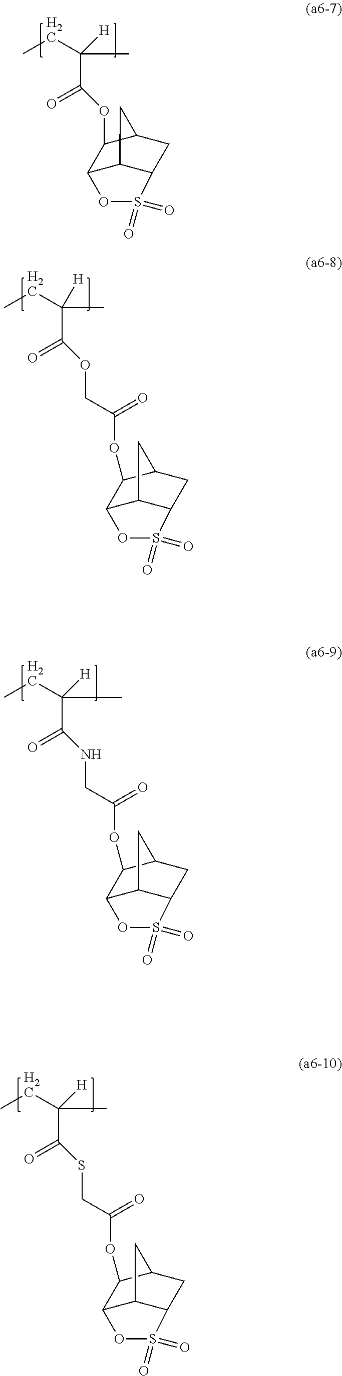

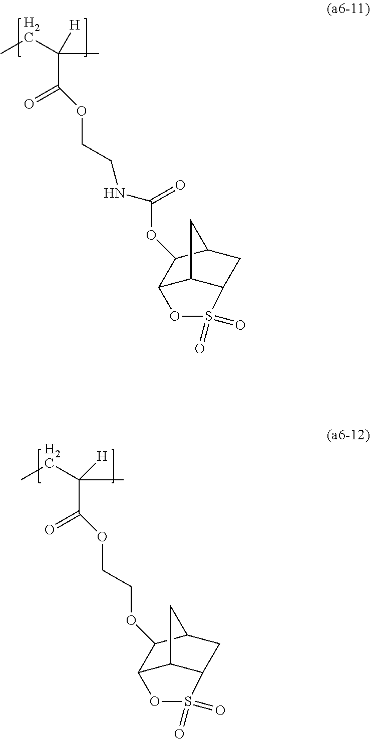

Among these, structural units represented by a formula (a6-1), a formula (a6-2), a formula (a6-6), a formula (a6-7), a formula (a6-8), and a formula (a6-12) are preferable, and structural units represented by a formula (a6-1), a formula (a6-2), a formula (a6-7), and a formula (a6-8) are more preferable.

When the resin (A) or the like includes the structural unit (a6), the content is preferably 1 to 50 mol %, more preferably 2 to 40 mol %, and still more preferably 3 to 30 mol %, based on all structural units of the resin (A) or the like.

<Structural Unit (a7)>

The resin (A) or the like may further include a structural unit that is decomposed upon exposure to radiation to generate an acid (hereinafter, may be referred to as “structural unit (a7)”). Specific examples of the structural unit (a7) include structural units described in JP-A-2016-79235, and it is preferable that the structural unit (a7) is a structural unit having a sulfonate anion or a carboxylate anion and an organic cation in a side chain, or a structural unit having a sulfonium cation and an organic anion in a side chain.

The structural unit having a sulfonic acid anion or a carboxylic acid anion and an organic cation in the side chain is preferably a structural unit represented by a formula (a7-A).

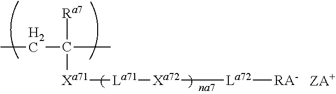

[In the formula (a7-A),
R^{a7} represents a hydrogen atom, a halogen atom, or an alkyl group having 1 to 6 carbon atoms and optionally having a halogen atom.

X^{a71} represents a single bond, —O—, —CO—, —S—, —SO—, —SO₂, —NR^{a71}—, a phenylene group optionally having a substituent, or a group obtained by combining these groups.

R^{a71} represents a hydrogen atom or an alkyl group having 1 to 6 carbon atoms.

L^{a71} and L^{a72} each independently represent a hydrocarbon group having 1 to 24 carbon atoms and optionally having a substituent, and —CH₂— included in the hydrocarbon group may be substituted with —O—, —S—, —CO, —SO—, or —SO₂—.

X^{a72} represents ***—CO—O—, ***—O—CO—, ***—O—CO—O—, ***—O—, and *** represents a binding site to L^{a71}.

na7 represents an integer of 0 to 2. When na7 is 2, a plurality of groups in parentheses may be the same as or different from each other.

RA⁻ represents a sulfonate anion or a carboxylate anion.

ZA⁺ represents an organic cation.]

Examples of the halogen atom and the alkyl group optionally having a halogen atom in R^{a7} include those that are the same as described as for R^{a01} and the like of the formula (a1-0).

R^{a7} is preferably a hydrogen atom or an alkyl group having 1 to 4 carbon atoms, more preferably a hydrogen atom or an alkyl group having 1 to 3 carbon atoms, still more preferably a hydrogen atom, a methyl group, or an ethyl group, and yet more preferably a hydrogen atom or a methyl group.

Examples of the alkyl group in R^{a71} include the same groups as those exemplified in R^{T} and the like.

X^{a71} is preferably a single bond, —O—, —CO—, —NR^{a71}—, a phenylene group optionally having a substituent, or a combination thereof, and more preferably a single bond, *—O—**, *—CO—O—**, *—O—CO—O—**, *—CO—NR^{a71}.**. NR^{a71}—CO—O—**, *—O—CO—NR^{a71}—** or *-Ax-Ph-Ay-**.

In the above group, * and ** represent a binding site, and * represents a binding site with the carbon atom to which R^{a7} is bonded. Ax and Ay each independently represent one or more bond species selected from the group consisting of a single bond, an ether bond, a thioether bond, an amide bond, an ester bond, and a carbonic acid ester bond. Ph represents a phenylene group optionally having a substituent.

When X^{a71} is a group represented by *-Ax-Ph-Ay-**, it is preferably a linking group represented by the following formula (X¹⁰).

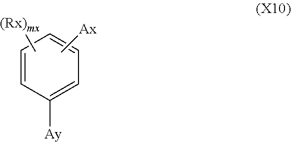

[In the formula (X10),
Ax represents bond species bonded to carbon atoms to which R^{a7} is bonded, and represents one or more bond species selected from the group consisting of a single bond, an ether bond, a thioether bond, an ester bond, a carbonic acid ester bond, and an amide bond.

Ay represents bond species bonded to L^{a7l}, and represents one or more species selected from the group consisting of a single bond, an ether bond, a thioether bond, an ester bond, a carbonic acid ester bond, and an amide bond.

Rx represents a halogen atom, a hydroxy group, an alkyl fluoride group having 1 to 6 carbon atoms, an alkyl group having 1 to 18 carbon atoms, or an alkoxy group having 1 to 6 carbon atoms,

mx represents an integer of 0 to 4, and when mx is an integer of 2 or more, a plurality of Rx may be the same as or different from each other.

When one of Ax and Ay is a single bond, the other is preferably one or more selected from the group consisting of an ether bond, a thioether bond, an ester bond, a carbonic acid ester bond, and an amide bond.

When either Ax or Ay is an amide bond, a bond represented by —CO—NR^{a71}— is preferable.

The binding site of Ay in the phenylene group is preferably the m-position or the p-position, and more preferably the p-position, with respect to the binding site of Ax.

Particularly, Rx is preferably a fluorine atom, an iodine atom, a trifluoromethyl group, a methyl group, or an ethyl group.

mx is preferably 0, 1, or 2.

Examples of X^{a71} include a single bond and groups represented by the following formula (X¹⁰-1) to formula (X¹⁰-10). X²⁰ represents —O— or —NR^{a71-}.

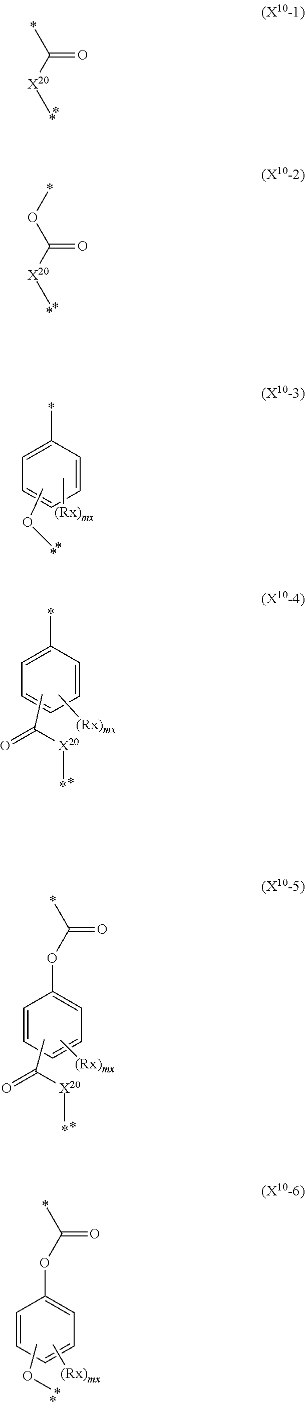

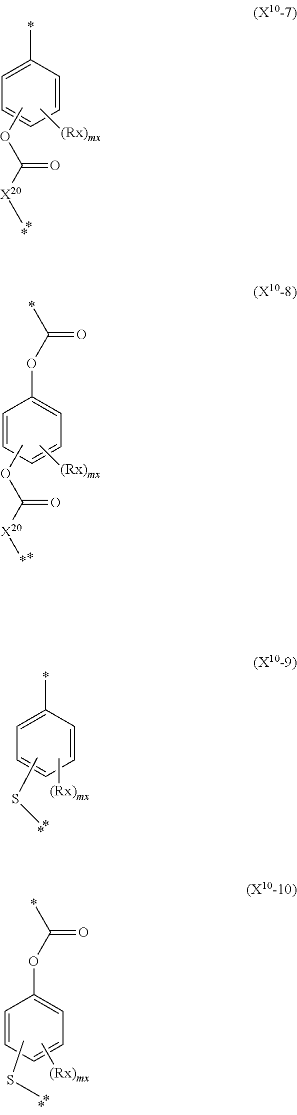

Specific examples of the groups represented by the formula (X¹⁰-1) to formula (X¹⁰-10) include the following groups.

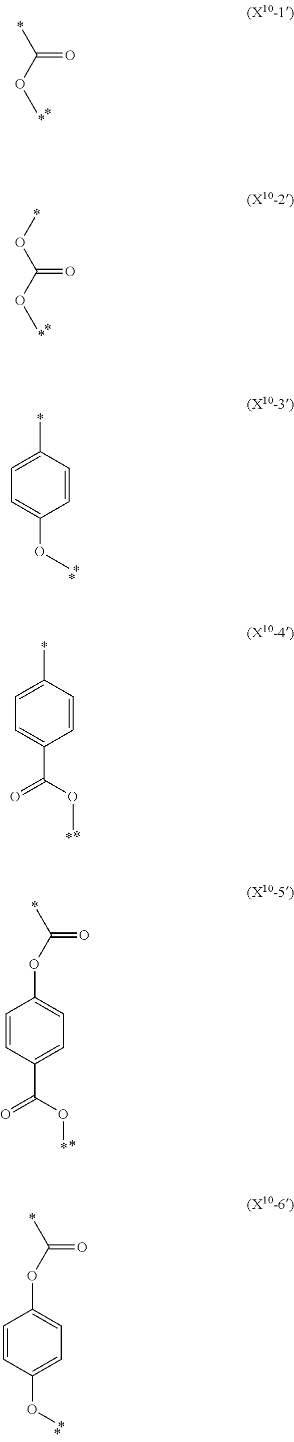

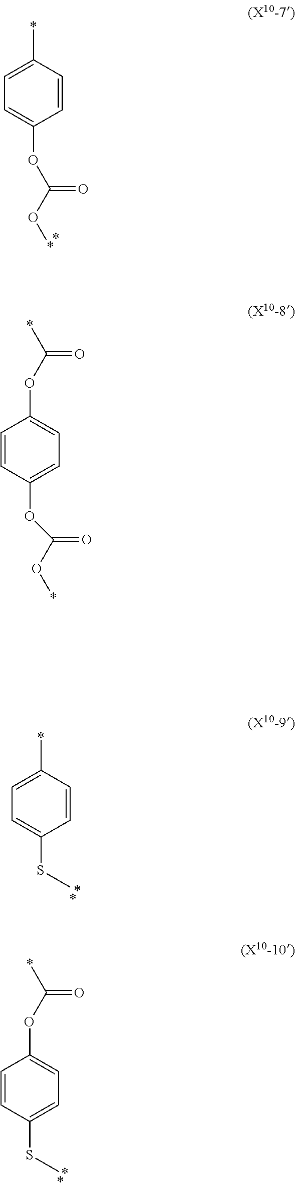

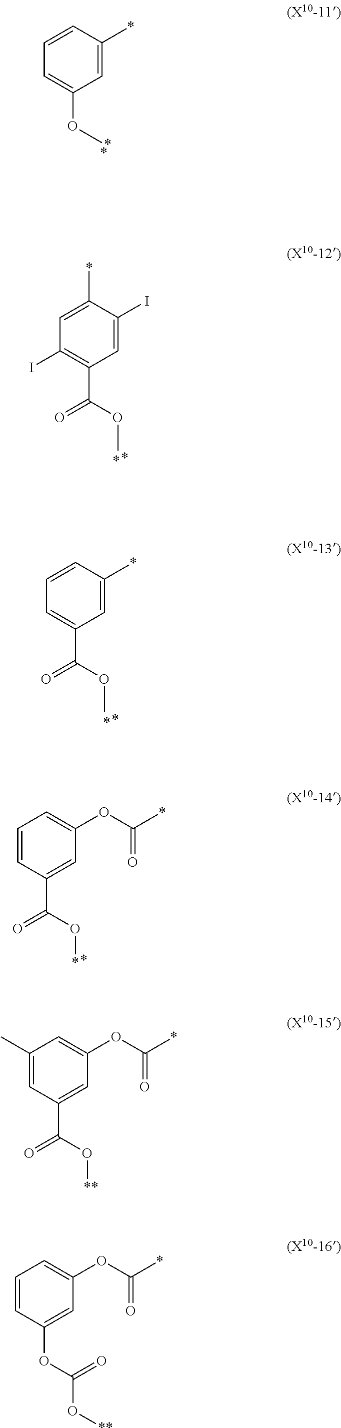

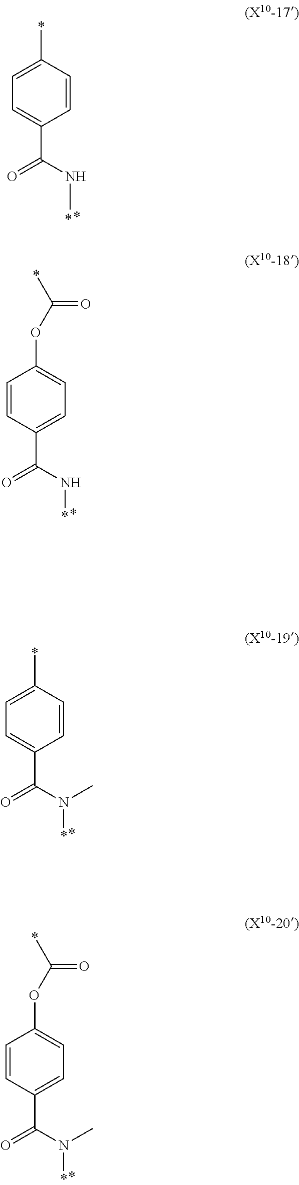 [SMILES: *c1ccc(C(=O)NC)cc1 ; *NC(=O)c1ccc(OC(*)=O)cc1 ; *c1ccc(C(=O)N(*)C)cc1 ; *C(=O)Oc1ccc(C(=O)N(*)C)cc1]

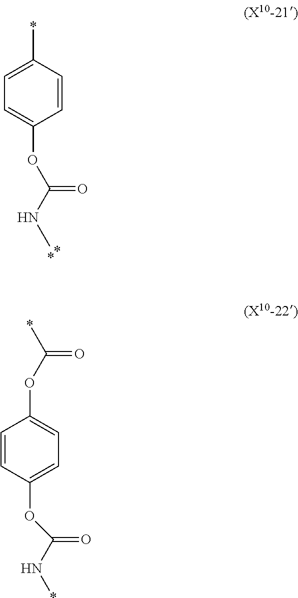 [SMILES: *c1ccc(OC(=O)NC)cc1 ; *NC(=O)Oc1ccc(OC(*)=O)cc1]

In particular, X^{a71} is preferably a single bond or a group represented by any one of a formula (X¹⁰-1′) and a formula (X¹⁰-3′) to a formula (X¹⁰-10′), more preferably a single bond or a group represented by any one of the formula (X¹⁰-1′), a formula (X¹⁰-4′), a formula (X¹⁰-5′), a formula (X¹⁰-6′), and a formula (X¹⁰-10′), and still more preferably a single bond, a group represented by the formula (X¹⁰-1′), a group represented by the formula (X¹⁰-5′), or a group represented by the formula (X¹⁰-6′).

Examples of the hydrocarbon group as for L^{a71} include groups which are obtained by removing one hydrogen atom from a chain hydrocarbon group, an alicyclic hydrocarbon group, an aromatic hydrocarbon group, and a group formed by combining two or more of these groups, and bonding to X^{a71} and X^{a72}. Examples of the hydrocarbon group as for L^{a72} include groups that are obtained by removing one hydrogen atom from a chain hydrocarbon group, an alicyclic hydrocarbon group, an aromatic hydrocarbon group, and a group formed by combining two or more of these groups, and bonding X^{a72} and RA⁻.

Examples of the chain hydrocarbon group as for L^{a71} and L^{a72} include groups obtained by removing one hydrogen atom of an alkyl group or an alkenyl group. The alkyl group may be either linear or branched, and specific examples thereof include a methyl group, an ethyl group, an n-propyl group, an iso-propyl group, an n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentadecyl group, and a heptadecyl group. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group, and a nonenyl group.

The number of carbon atoms of the chain hydrocarbon group is preferably 1 to 20, more preferably 1 to 18, still more preferably 1 to 10 carbon atoms.

Examples of the alicyclic hydrocarbon group as for L^{a71} and L^{a72} include groups obtained by removing one hydrogen atom of a monocyclic or polycyclic cycloalkyl group. Examples of the monocyclic cycloalkyl group include a cyclobutyl group, a cycloheptyl group, a cyclohexyl group, a cyclopentyl group, and a cyclooctyl group.

Examples of the polycyclic cycloalkyl group as for L^{a7l} and L^{a72} include a cycloalkyl group having a crosslinked structure, a cycloalkyl group in which two or more rings are fused, or a cycloalkyl group in which two rings are bonded by spiro bonding. Examples of the cycloalkyl group having a crosslinked structure include a norbornyl group and an adamantyl group. Examples of the cycloalkyl group in which two or more rings are fused include a bicyclo[4.4.0]decanyl group and a steroid group (steroid skeleton). Examples of the cycloalkyl group in which two rings are bonded by spiro bonding include a spirocyclic cycloalkyl group in which one cycloalkyl group selected from the group consisting of a cyclopentyl group, a cyclohexyl group, a norbornyl group, and an adamantyl group, and a cycloalkyl group having 5 to 8 carbon atoms are bonded by spiro bonding. A double bond may be formed between two carbon atoms included in the alicyclic hydrocarbon group. More specifically, alicyclic hydrocarbon groups represented by the following formulas are exemplified.

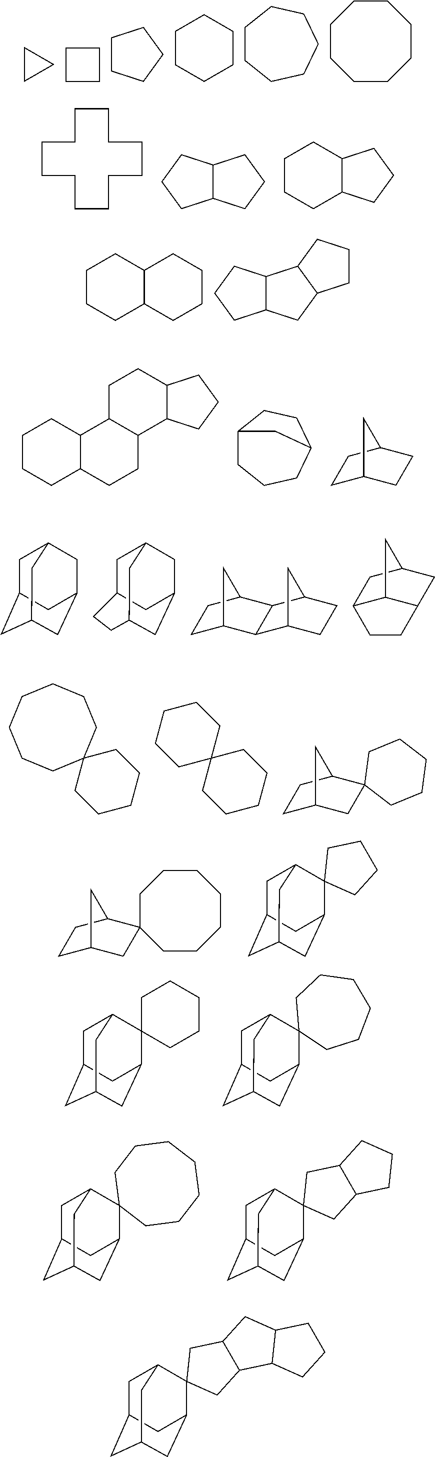

When the alicyclic hydrocarbon group is a monocyclic cycloalkyl group, the number of carbon atoms of the alicyclic hydrocarbon group is preferably 3 to 18, more preferably 3 to 12, and still more preferably 3 to 8. When the alicyclic hydrocarbon group is a polycycic cycloalkyl group, the number of carbon atoms of the alicyclic hydrocarbon group is preferably 6 to 18, and more preferably 7 to 12.

Examples of the aromatic hydrocarbon group L^{a71} and L^{a72} include groups obtained by removing one hydrogen atom of an aryl group. Examples of the aryl group include a phenyl group, a naphthyl group, an anthryl group, a biphenyl group, and a fluorenyl group. The number of carbon atoms of the aromatic hydrocarbon group is preferably 5 to 14, more preferably 6 to 14, and still more preferably 6 to 10.

Examples of the groups obtained by combining two or more groups of the chain hydrocarbon group, the alicyclic hydrocarbon group, and the aromatic hydrocarbon group as for L^{a71} and L^{a72} include a group obtained by combining the chain hydrocarbon group with the alicyclic hydrocarbon group, a group obtained by combining the chain hydrocarbon group with the aromatic hydrocarbon group, a group obtained by combining the alicyclic hydrocarbon group with the aromatic hydrocarbon group, and a group obtained by combining the chain hydrocarbon group, the alicyclic hydrocarbon group, and the aromatic hydrocarbon group. The group obtained by combining the alicyclic hydrocarbon group with the aromatic hydrocarbon group may also be a fused ring.

When —CH₂— included in the hydrocarbon group as for L^{a71} and L^{a72} is substituted with —O—, —CO—, —S—, —SO—, or —SO₂—, the number of carbon atoms before substitution is defined as the total number of the hydrocarbon group.

Among the hydrocarbon groups of L^{a71} and L^{a72}, examples of the groups in which —CH₂— in a chain hydrocarbon group is substituted with —O—, —CO—, —S—, —SO—, or —SO₂— include, as described above, a hydroxyl group, a carboxyl group, a thiol group, a carbonyl group, an oxy group, a thio group, a sulfonyl group, an alkoxy group, an alkoxycarbonyl group, an alkylcarbonyl group, an alkylcarbonyloxy group, an alkoxycarbonyloxy group,
an alkanediyloxy group, an alkanediyloxycarbonyl group, an alkanediylcarbonyl group, and an alkanediylcarbonyloxy group. Examples of these substituted groups include those that are the same as described herein as long as the upper limit of the number of carbon atoms is acceptable. The number of carbon atoms in these substituted groups is, for example, 1 to 23, 1 to 17, preferably 1 to 11, more preferably 1 to 6, still more preferably 1 to 4, and still more preferably 1 to 3 in the case of an alkoxy group or the like, 2 to 23, 2 to 17, preferably 2 to 11, more preferably 2 to 6, still more preferably 2 to 4, and still more preferably 2 or 3 in the case of an alkoxycarbonyl group or the like, 2 to 24, 2 to 18, preferably 2 to 12, more preferably 2 to 6, still more preferably 2 to 4, and still more preferably 2 or 3 in the case of an alkylcarbonyl group or the like, and 2 to 22, 2 to 16, preferably 2 to 10, more preferably 2 to 6, and still more preferably 2 to 4, and yet more preferably 2 or 3 in the case of an alkoxycarbonyloxy group.

Examples of the group in which —CH₂— included in an alicyclic hydrocarbon group, an aromatic hydrocarbon group, or a group obtained by combining these groups among hydrocarbon groups of L^{a71} and L^{a72} is substituted with —O—, —CO—, —S—, —SO—, or —SO₂— include a group including a cyclic ether, a cyclic ketone, a cyclic ester (lactone), a cyclic thioether, a cyclic acetal, a cyclic sulfonic acid ester (sultone) structure, and a group derived from a furan ring or a thiophene ring. Specific examples include those that are the same as described herein as long as the upper limit of the number of carbon atoms is acceptable.

Examples of the substituent that may be possessed by the hydrocarbon group as for L^{a71} and L^{a72} include a halogen atom, a cyano group, and a nitro group. Examples of the halogen atom include a fluorine atom, a chlorine atom, a bromine atom, and an iodine atom.

When L^{a71} and L^{a72} are groups obtained by combining an alicyclic hydrocarbon group or an aromatic hydrocarbon group with a chain hydrocarbon group, the chain hydrocarbon group may be substantially regarded as a substituent that is possessed by the alicyclic hydrocarbon group or the aromatic hydrocarbon group. In addition, —CH₂— of the chain hydrocarbon group included in the hydrocarbon group as for La^{T1} and La⁷² is substituted with —O—, —CO—, —S—, —SO—, or —SO₂—, whereby the hydrocarbon group as for L^{a71} and L^{a72} can substantially have substituents such as a hydroxy group, a carboxy group, an alkoxy group, an alkoxycarbonyl group, an alkylcarbonyl group, an alkylcarbonyloxy group, a thiol group, an alkylthio group, and an alkylsulfonyl group.

na7 is preferably 0 or 1.

Examples of ZA⁺ in the formula (a7-A) include the same cations as in the salt represented by a formula (B1) or the like.

In the formula (a7-A), when the hydrocarbon group as for L^{a71} and L^{a72} is a saturated hydrocarbon group, it is possible to include the same groups as described as divalent linking group as for A^{b7} in a formula (a7-B) described below.

Examples of the structural unit represented by the formula (a7-A) also include a structural unit or the like represented by a formula (a7-A1).

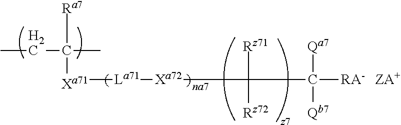

[In the formula (a7-A1),

R^{a7}, X^{a71}, L^{a71}, X^{a72}, na7, RA⁻, and ZA⁺ are the same as defined above.

z7 represents an integer of 0 to 6.

Q^{a7}, Q^{b7}, R^{z71}, and R^{z72} each independently represent a hydrogen atom, a fluorine atom, a perfluoroalkyl group having 1 to 6 carbon atoms or an alkyl group having 1 to 6 carbon atoms, and when z7 is 2 or more, a plurality of R^{z71} and R^{z72} may be the same as or different from each other.

Examples of the perfluoroalkyl group having 1 to 6 carbon atoms or alkyl group having 1 to 6 carbon atoms in Q^{a7}, Q^{b7}, R^{z71}, and R^{z72} include the same groups as described for R^{a7}.

Examples of the structural unit represented by the formula (a7-A) include the following structural units, structural units in which the group corresponding to the methyl group as for R^{a7} is substituted with a hydrogen atom, a halogen atom (for example, a fluorine atom) or an alkyl group having 1 to 6 carbon atoms and optionally having a halogen atom (for example, a trifluoromethyl group), and structural units described in WO 2012/050015 A. ZA⁺ represents an organic cation.

[chemical structure]

[chemical structure]

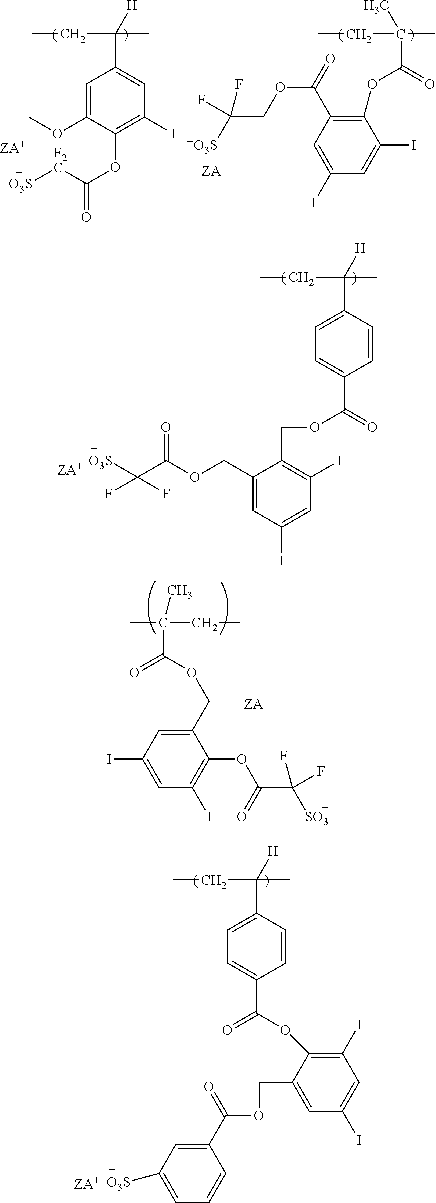

The structural unit having a sulfonio group and an organic anion in the side chain is preferably a structural unit represented by the formula (a7-B).

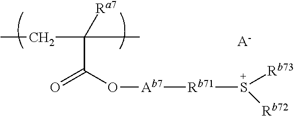

[In the formula (a7-B),

R^{a7} represents a hydrogen atom, a halogen atom, or an alkyl group having 1 to 6 carbon atoms and optionally having a halogen atom.

A^{b7} represents a single bond or a divalent linking group.

R^{b71} represents a divalent aromatic hydrocarbon group having 6 to 18 carbon atoms and optionally having a substituent.

R^{b72} and R^{b73} each independently represent a hydrocarbon group having 1 to 18 carbon atoms and optionally having a substituent, and R^{b72} and R^{b73} may be bonded to each other to form a ring together with a sulfur atom to which R^{b72} and R^{b73} are bonded.

A⁻ represents an organic anion.]

Examples of the halogen atom and the alkyl group optionally having a halogen atom as for R^{a7} include the same halogen atoms and the alkyl groups optionally having a halogen atom of the formula (a7-A).

Examples of the divalent aromatic hydrocarbon group having 6 to 18 carbon atoms represented by R^{b71} include a phenylene group and a naphthylene group.

Examples of the hydrocarbon group represented by R^{b72} and R^{b73} include an alkyl group, an alicyclic hydrocarbon group, an aromatic hydrocarbon group, and groups formed by combining these groups.

Examples of the alkyl group, the alicyclic hydrocarbon group, the aromatic hydrocarbon group, and the groups formed by combining these groups include the same groups as described above.

Examples of the divalent linking group represented by A^{b7} include a divalent saturated hydrocarbon group having 1 to 18 carbon atoms, and —CH₂— included in the divalent saturated hydrocarbon group may be substituted with —O—, —S—, —SO—, —SO₂—, or —CO—.

Examples of the divalent saturated hydrocarbon group include divalent chain saturated hydrocarbon groups such as a linear or branched alkanediyl group, a monocycic or polycycic divalent alicyclic saturated hydrocarbon group, or a combination thereof.

Specific examples thereof include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; branched alkanediyl groups such as a butane-1,3-diyl group, a 2-methylpropane-1,3-diyl group, a 2-methylpropane-1,2-diyl group, a pentane-1,4-diyl group, and a 2-methylbutane-1,4-diyl group; divalent monocyclic alicyclic saturated hydrocarbon groups that are monocyclic cycloalkanediyl groups, such as a cyclobutane-1,3-diyl group, a cyclopentane-1,3-diyl group, a cyclohexane-1,4-diyl group, and a cyclooctane-1,5-diyl group; and divalent polycyclic alicyclic saturated hydrocarbon groups that are polycyclic cycloalkanediyl groups, such as a norbornane-1,4-diyl group, a norbornane-2,5-diyl group, an adamantane-1,5-diyl group, and an adamantane-2,6-diyl group.

Those in which —CH₂— included in the saturated hydrocarbon group are substituted with —O—, —S—, —SO—, —SO₂—, or —CO— include, for example, divalent groups shown below. Before substituting —CH₂— included in the saturated hydrocarbon group by —O—, —S—, —SO—, —SO₂—, or —CO—, the number of carbon atoms is 17 or less. In the following formulas, * and ** represent a binding site, and * represents a binding site to R^{b71}.

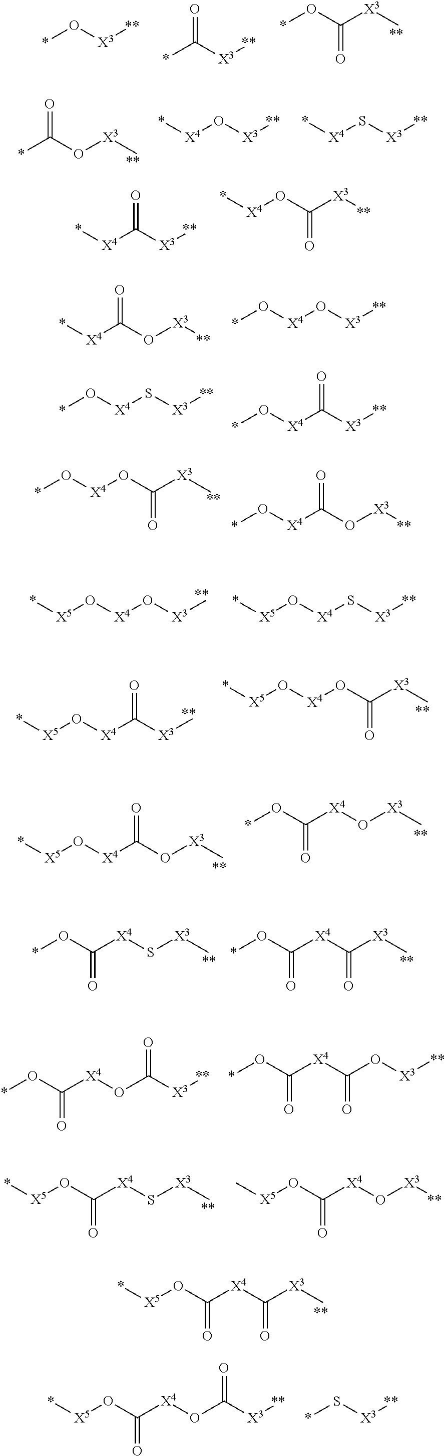

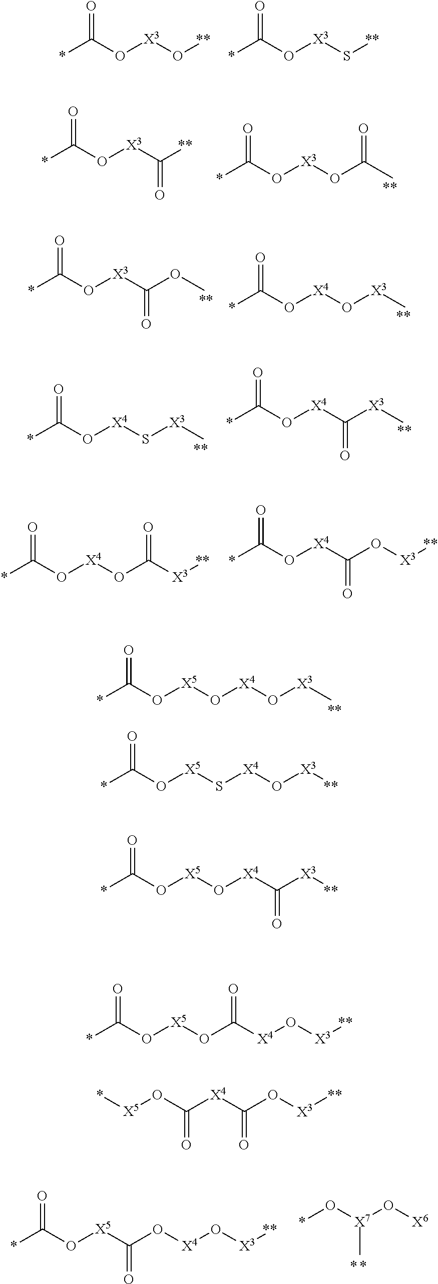

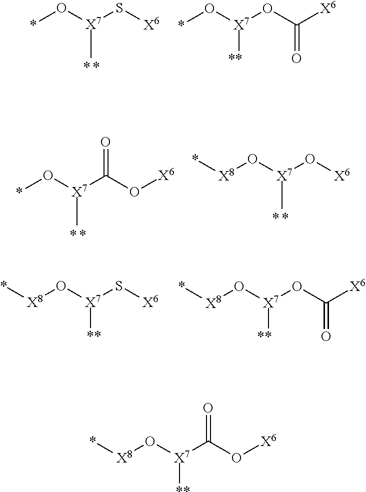

[In the formula, X³ represents a divalent saturated hydrocarbon group having 1 to 16 carbon atoms.

X⁴ represents a divalent saturated hydrocarbon group having 1 to 15 carbon atoms.

X⁵ represents a divalent saturated hydrocarbon group having 1 to 13 carbon atoms.

X⁶ represents a divalent saturated hydrocarbon group having 1 to 14 carbon atoms.

X⁷ represents a trivalent saturated hydrocarbon group having 1 to 14 carbon atoms.

X⁸ represents a divalent saturated hydrocarbon group having 1 to 13 carbon atoms.

Examples of the structural unit including a cation in the formula (a7-B) include the following structural units and structural units in which a group corresponding to a methyl group of R^{a7} is substituted with a hydrogen atom, a halogen atom (for example, a fluorine atom), or an alkyl group having 1 to 6 carbon atoms and optionally having a halogen atom (for example, a trifluoromethyl group).

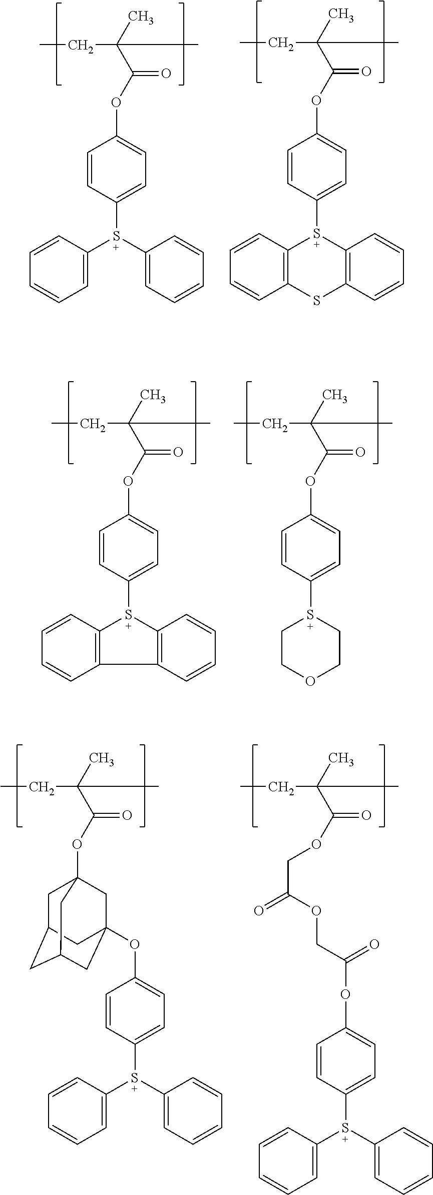

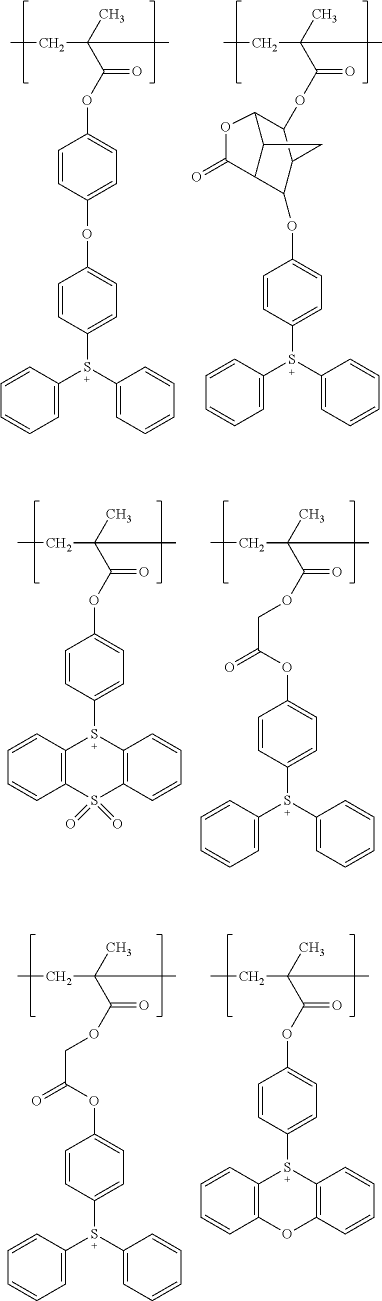

Examples of the organic anion represented by A⁻ include a sulfonic acid anion, a sulfonylimide anion, a sulfonylmethide anion, and a carboxylic acid anion. The organic anion represented by A⁻ is preferably a sulfonic acid anion, and the sulfonic acid anion is more preferably the same as the anion exemplified in the compound (B). Examples of the sulfonylimide anion, the sulfonylmethide anion, and the carboxylic acid anion include those that are the same as anions to be described in the compound (B).

Examples of the structural unit represented by the formula (a7-B) include the following.

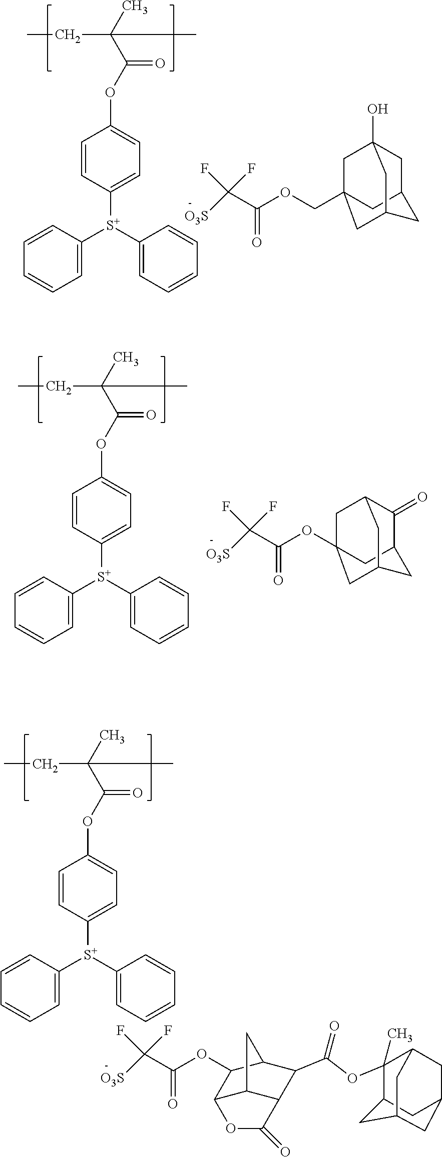

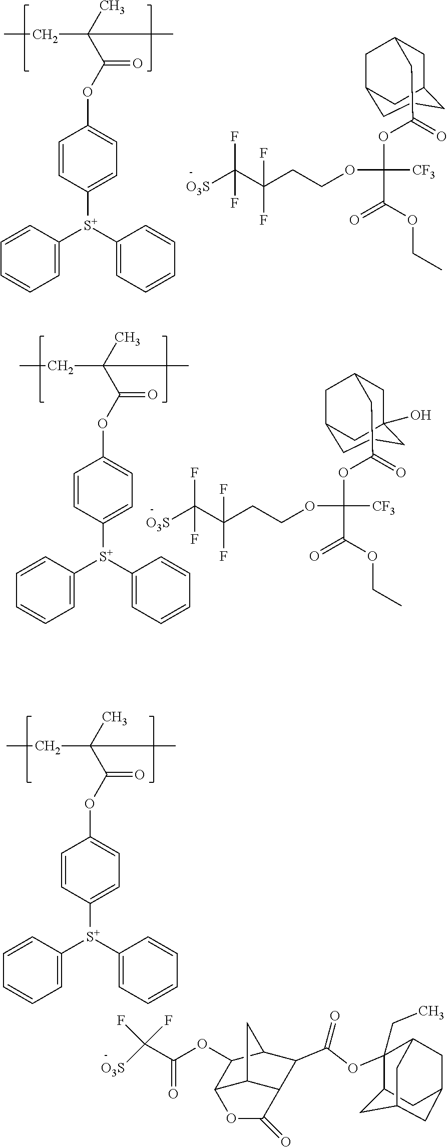

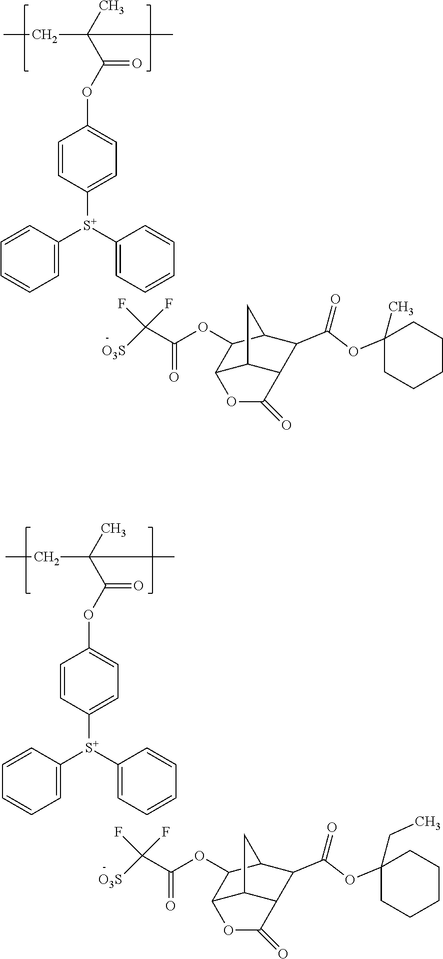

When the structural unit (a7) is included in the resin (A) or the like, the content of the structural unit (a7) is preferably 1 to 30 mol %, more preferably 2 to 25 mol %, and still more preferably 3 to 20 mol %, based on all structural units of the resin (A) or the like.

The resin (A) or the like is preferably a resin including the structural unit (a1). Particularly, the resin (Ap) is more preferably a resin composed of a structural unit (IP), the structural unit (a1) and a structural unit (s), that is, a copolymer of a salt (I), a monomer (a1), and a monomer (s). The resin (A) including no structural unit (IP) is preferably a resin composed of the structural unit (a1) and the structural unit (s), that is, a copolymer of the monomer (a1) and the monomer (s).

The structural unit (a1) is preferably at least one selected from the group consisting of the structural unit (a1-0), the structural unit (a1-1), the structural unit (a1-2) (preferably the structural unit having a cyclohexyl group or a cyclopentyl group), a structural unit represented by a formula (a1-4), a structural unit represented by a formula (a1-5), and a structural unit represented by a formula (a1-6), more preferably at least two, and still more preferably at least two selected from the group consisting of the structural unit (a1-1) and the structural unit (a1-2).

The structural unit (s) is preferably at least one selected from the group consisting of the structural unit (a2) and the structural unit (a3). The structural unit (a2) is preferably a structural unit (a2-C) or the structural unit (a2-A). The structural unit (a3) is preferably at least one selected from the group consisting of a structural unit represented by a formula (a3-1), a structural unit represented by a formula (a3-2), and a structural unit represented by a formula (a3-4).

The respective structural units constituting the resin (A) or the like may be used singly, or two or more structural units may be used in combination, and using a monomer from which these structural units are derived, it is possible to produce by a known polymerization method (for example, radical polymerization method). The content of the respective structural units included in the resin (A) or the like can be adjusted according to the amount of the monomer used in the polymerization.

The resin (A) and the resin (Ap) may have, for example, a weight-average molecular weight of preferably 2,000 or more (more preferably 2,500 or more, and still more preferably 3,000 or more), and 50,000 or less (more preferably 30,000 or less, and still more preferably 15,000 or less), and may also include an oligomer having a weight-average molecular weight of less than 2,000. As used herein, the weight-average molecular weight is a value determined by gel permeation chromatography under the analysis conditions described in Examples. The structural unit (IP) may constitute a dimer, a trimer, and a compound or oligomer having a weight-average molecular weight of less than 2,000.

### [Acid Generator]

The acid generator of the present invention is an acid generator including the salt (I) or the structural unit (IP) of the present invention or the structural unit (IP). The structural unit (IP) can be included as a compound or a resin (Ap) obtained by polymerizing a plurality thereof. In the acid generator, the salt (I) may be included singly, or two or more of salts (I) may be included. The compound or resin (Ap) including the structural unit (IP) may be used singly, or two or more thereof may be used in combination. In addition, the acid generator of the present invention may include both the salt (I) and the structural unit (IP).

The acid generator of the present invention may include, in addition to the salt (I) and/or the structural unit (IP), a known compound acting as an acid generator in the resist field (hereinafter, may be referred to as “compound (B)” or “acid generator (B)”). The compound (B) may be used singly, or two or more thereof may be used in combination. The compound (B) will be described later.

When the acid generator includes the salt (I) and the compound (B), a ratio of the content of the salt (I) to that of the compound (B) (mass ratio, salt (I):compound (B)) is typically 1:99 to 99:1, preferably 2:98 to 98:2, more preferably 5:95 to 95:5, still more preferably 10:90 to 90:10, yet more preferably 15:85 to 85:15, and particularly preferably 40:60 to 60:40.

When the acid generator includes the structural unit (IP) and the compound (B), a ratio of the content of the structural unit (IP) to that of the compound (B) (mass ratio, structural unit (IP):compound (B)) is typically 1:99 to 99:1, preferably 2:98 to 98:2, more preferably 5:95 to 95:5, still more preferably 10:90 to 90:10, yet more preferably 15:85 to 85:15, and particularly preferably 40:60 to 60:40.

### [Resist Composition]

The resist composition of the present invention includes the acid generator of the present invention. The acid generator herein may be either the salt (I) or the structural unit (IP).

When the resist composition of the present invention includes the salt (I), the content of the salt (I) is preferably 0.1 mass % or more and 99.9 mass % or less, more preferably 1 mass % or more and 45 mass % or less, still more preferably 1 mass % or more and 40 mass % or less, and yet more preferably 2 mass % or more and 35 mass % or less based on the solid content of the resist composition. When the resist composition includes the resin (A) or the like, the content of the salt (I) is preferably 1 part by mass or more and 45 parts by mass or less, more preferably 1 part by mass or more and 40 parts by mass or less, and still more preferably 2 parts by mass or more and 35 parts by mass or less, based on 100 parts by mass of the resin (A) or the like.

In the resist composition of the present invention, the content of the resin (Ap) is preferably 60 mass % or more and 99 mass % or less, more preferably 70 mass % or more and 99 mass % or less, still more preferably 80 mass % or more and 99 mass % or less, and yet more preferably 90 mass % or more and 99 mass % or less based on the solid content of the resist composition.

The resist composition of the present invention may include, in addition to the acid generator of the present invention, a resin. The resin may be either a resin including the structural unit (a1) having an acid-labile group, or a resin including no structural unit (a1). The resist composition of the present invention preferably includes a resin including the structural unit (a1) having an acid-labile group. That is, the resist composition preferably includes at least:
(a) the salt (I) and the resin (A) including the structural unit (a1) having an acid-labile group;
(b) the resin (Ap) including the structural unit (IP) and the structural unit (a1) having an acid-labile group; or
(c) the resin (Ap) including the structural unit (IP) and the resin (A) including the structural unit (a1) having an acid-labile group.

Among these, the resist composition is preferably the resist composition (b). Two or more resins (A) and/or resins (Ap) may be included.

It is preferable that the resist composition of the present invention further includes a quencher (hereinafter, may be referred to as “quencher (C)”) and/or a solvent (hereinafter, may be referred to as “solvent (E)”). In addition, the resist composition of the present invention may further include a resin other than the above-described resin (A) or the like.

<Resin Other than Resin (A) or the Like>

The resist composition of the present invention may include a resin other than the resin (Ap) and the resin (A). Examples of the resin other than the resin (Ap) and the resin (A) include a resin including the same structural unit as that of the resin (A), except that no structural unit (a1) is included in the above-described resin (A) (hereinafter, may be referred to as “resin (AX)”), and a resin including the structural unit (a4) and/or the structural unit (a5) (including neither structural unit (IP) nor structural unit (a1), hereinafter, may be referred to as “resin (X)”).

Examples of the resin (AX) include a resin including the structural unit (a2), and a resin including the structural unit (a2-A) is preferable. In the resin (AX), the content of the structural unit (a2-A) is preferably 5 mol % or more, more preferably 10 mol % or more, and still more preferably 15 mol % or more. In addition, the content is preferably 80 mol % or less, and more preferably 70 mol % or less.

Examples of the structural unit that may be further included in the resin (X) include the structural unit (a2), the structural unit (a3), and structural units derived from other known monomers. Particularly, the resin (X) is preferably a resin composed only of the structural unit (a4) and/or the structural unit (a5), and more preferably a resin composed only of the structural unit (a4).

When the resin (X) includes the structural unit (a4), the content is typically 20 mol % or more, preferably 30 mol % or more, more preferably 40 mol % or more, and still more preferably 45 mol % or more, based on all structural units of the resin (X). In addition, the content is typically 100 mol % or less, preferably 80 mol % or less, more preferably 70 mol % or less, still more preferably 60 mol % or less, and yet more preferably 55 mol % or less. Specifically, the content is typically 20 to 100 mol %, preferably 20 to 80 mol %, more preferably 30 to 70 mol %, still more preferably 40 to 60 mol %, and yet more preferably 45 to 55 mol %. When the resin (X) includes the structural unit (a5), the content is typically 20 mol % or more, preferably 30 mol % or more, more preferably 40 mol % or more, and still more preferably 45 mol % or more, based on all structural units of the resin (X). In addition, the content is typically 100 mol % or less, preferably 80 mol % or less, more preferably 70 mol % or less, still more preferably 60 mol % or less, and yet more preferably 55 mol % or less. Specifically, the content is typically 20 to 100 mol %, preferably 20 to 80 mol %, more preferably 30 to 70 mol %, still more preferably 40 to 60 mol %, and yet more preferably 45 to 55 mol %. In addition, when the resin (X) includes the structural unit (a4) and the structural unit (a5), the total content is typically 40 mol % or more, preferably 60 mol % or more, more preferably 70 mol % or more, and still more preferably 80 mol % or more, based on all structural units of the resin (X). Further, the total content is 100 mol % or less. Specifically, the total content is typically 40 to 100 mol %, preferably 60 to 100 mol %, more preferably 70 to 100 mol %, and still more preferably 80 to 100 mol %.

The respective structural unit constituting the resin (AX) and the resin (X) may be used singly, or two or more structural units may be used in combination, and using a monomer from which these structural units are derived, the production can be performed by a known polymerization method (for example radical polymerization method). The content of the respective structural units included in the resin (AX) and the resin (X) can be adjusted according to the amount of the monomer used in the polymerization.

The resin (AX) and the resin (X) preferably have a weight-average molecular weight of 6,000 or more (more preferably 7,000 or more) and 80,000 or less (more preferably 60,000 or less), and may also include an oligomer having a weight-average molecular weight of less than 6,000. The measurement means of the weight-average molecular weight of the resin (AX) and the resin (X) is the same as in the case of the resin (A) or the like.

When the resist composition of the present invention includes the resin (X), the content is preferably 1 to 60 parts by mass, more preferably 1 to 50 parts by mass, still more preferably 1 to 40 parts by mass, yet more preferably 1 to 30 parts by mass, and further preferably 1 to 8 parts by mass, based on 100 parts by mass of the total of the resin (A) or the like.

The content of the resin (A) or the like in the resist composition is preferably 60 mass % or more and 99 mass % or less, more preferably 70 mass % or more and 99 mass % or less, still more preferably 80 mass % or more and 99 mass % or less, and yet more preferably 90 mass % or more and 99 mass % or less, based on the solid content of the resist composition. The content of the resin (Ap) is preferably 60 mass % or more and 99 mass % or less, more preferably 70 mass % or more and 99 mass % or less, still more preferably 80 mass % or more and 99 mass % or less, and yet more preferably 90 mass % or more and 99 mass % or less, based on the solid content of the resist composition. In addition, when resins other than the resin (A) or the like are included, the total content of the resin (A) or the like and resins other than the resin (A) or the like is preferably 60 mass % or more and 99 mass % or less, more preferably 70 mass % or more and 99 mass % or less, still more preferably 80 mass % or more and 99 mass % or less, and yet more preferably 90 mass % or more and 99 mass % or less, based on the solid content of the resist composition. The solid content of the resist composition and the content of the resin thereto can be measured by a known analysis means such as liquid chromatography or gas chromatography.

### <Compound (B)>

Either nonionic compound or ionic compound may be used as the compound (B). Examples of the nonionic compound include sulfonate esters (for example, 2-nitrobenzyl ester, aromatic sulfonate, oxime sulfonate, N-sulfonyloxyimide, sulfonyloxyketone, diazonaphthoquinone 4-sulfonate), and sulfones (for example, disulfone, ketosulfone, sulfonyldiazomethane). Typical examples of the ionic compound include onium salts containing an onium cation (for example, diazonium salt, phosphonium salt, sulfonium salt, iodonium salt). Examples of the anion of the onium salt include a sulfonate anion, a sulfonylimide anion, a sulfonylmethide anion, and a carboxylate anion.

In addition, the compound (B) may be a part of the structural unit constituting the resin included in the resist composition of the present invention as in the structural unit (a7) exemplified herein.

Specific examples of the compound (B) include compounds generating an acid upon exposure to radiation described in JP-A-63-26653, JP-A-55-164824, JP-A-62-69263, JP-A-63-146038, JP-A-63-163452, JP-A-62-153853, JP-A-63-146029, U.S. Pat. Nos. 3,779,778, 3,849,137, DE Patent No. 3914407, and EP Patent No. 126,712. In addition, compounds produced by a known method may be used. The compound (B) may be used singly, or two or more thereof may be used in combination.

The compound (B) may be an acid generator having an acid-labile group. For example, the compound or resin serving as the acid generator may include a partial structure serving as the acid-labile group, and the acid-labile group preferably includes the group (1) or group (2) exemplified in the present specification. When the resist composition of the present invention includes a compound having an acid-labile group, the resin including a structural unit having an acid-labile group need not be included.

Preferable examples of the compound (B) include a salt represented by the formula (B1) (hereinafter, may be referred to as “salt (B1)” or “compound (B1)”), a salt represented by the formula (B2) (hereinafter, may be referred to as “salt (B2)” or “compound (B2)”), and a salt represented by the formula (B4) (hereinafter, may be referred to as “salt (B4)” or “compound (B4)”).

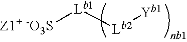

[In the formula (B1),
L^{b1} represents a single bond or an (nb1+1)-valent hydrocarbon group optionally having a substituent, and —CH₂— included in the hydrocarbon group may be substituted with —O—, —S—, —CO—, —SO—, or —SO₂—.

L^{b2} represents a single bond or a divalent hydrocarbon group having 1 to 24 carbon atoms and optionally having a substituent, and —CH₂— included in the hydrocarbon group may be substituted with —O—, —S—, —CO, —SO—, or —SO₂—.

Y^{b1} represents a methyl group optionally having a substituent or a cyclic hydrocarbon group having 3 to 24 carbon atoms and optionally having a substituent, and —CH₂— included in the cyclic hydrocarbon group may be substituted with —O—, —S—, —SO—, —SO₂— or —CO—. nb1 represents an integer of 1 to 6. When nb1 is 2 or more, a plurality of groups in parentheses may be the same as or different from each other.

Z1⁺ represents an organic cation.]

Examples of the (nb1+1)-valent hydrocarbon group as for L^{b1} include groups that are obtained by removing nb1 hydrogen atoms from a monovalent chain hydrocarbon group, a monovalent alicyclic hydrocarbon group, a monovalent aromatic hydrocarbon group, and a monovalent group formed by combining these two or more groups, and bonding to one or more L^{b2}. The number of carbon atoms of the hydrocarbon group is preferably 1 to 48, more preferably 1 to 42, still more preferably 1 to 36, yet more preferably 1 to 30, and further preferably 1 to 24.

Examples of the chain hydrocarbon group include groups obtained by removing nb1 hydrogen atoms of the alkyl group or alkenyl group. The alkyl group may be either linear or branched, and specific examples thereof include a methyl group, an ethyl group, an n-propyl group, an iso-propyl group, an n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentadecyl group, and a heptadecyl group. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group, and a nonenyl group.

The number of carbon atoms of the chain hydrocarbon group is preferably 1 to 36, more preferably 1 to 24, still more preferably 1 to 20, yet more preferably 1 to 18, further preferably 1 to 12, and still further preferably 1 to 10.

Examples of the alicyclic hydrocarbon group include groups obtained by removing nb1 hydrogen atoms of the monocyclic or polycyclic cycloalkyl group. Examples of the monocyclic cycloalkyl group include a cyclobutyl group, a cyclopentyl group, a cyclohexyl group, a cycloheptyl group, and a cyclooctyl group.

Examples of the polycyclic cycloalkyl group include a cycloalkyl group having a crosslinked structure, a cycloalkyl group in which two or more rings are fused, or a cycloalkyl group in which two rings are bonded by spiro bonding. Examples of the cycloalkyl group having a crosslinked structure include a norbornyl group and an adamantyl group. Examples of the cycloalkyl group in which two or more rings are fused include a bicyclo[4.4.0]decanyl group and a steroid group (steroid skeleton). Examples of the cycloalkyl group in which two rings are bonded by spiro bonding include a spirocyclic cycloalkyl group in which one cycloalkyl group selected from the group consisting of a cyclopentyl group, a cyclohexyl group, a norbornyl group, and an adamantyl group, and a cycloalkyl group having 5 to 8 carbon atoms are bonded by spiro bonding. A double bond may be formed between two carbon atoms included in the alicyclic hydrocarbon group.

More specifically, alicyclic hydrocarbon groups represented by the following formulas are exemplified.

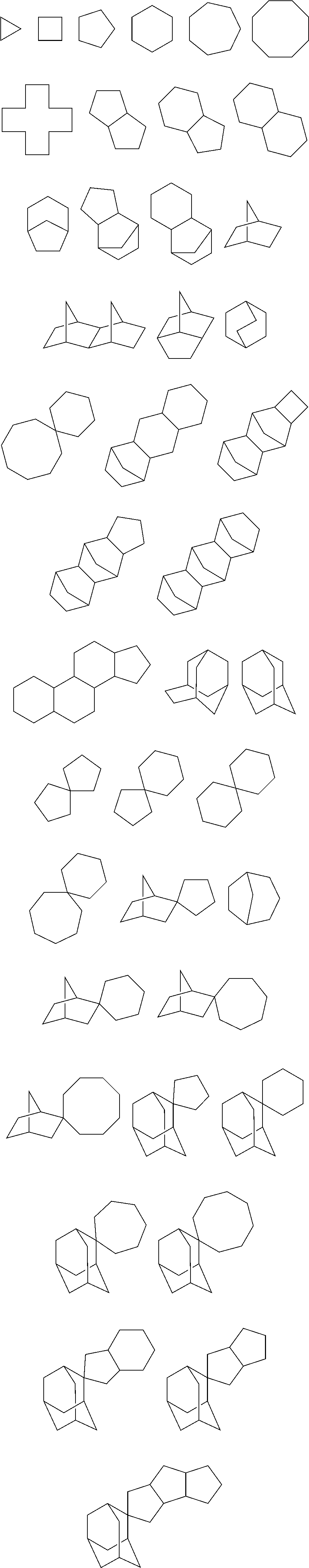

When the alicyclic hydrocarbon group is a monocyclic cycloalkyl group, the number of carbon atoms of the alicyclic hydrocarbon group is preferably 3 to 24, more preferably 3 to 20, still more preferably 3 to 18, yet more preferably 3 to 12, further preferably 3 to 10, and still further preferably 3 to 8. When the alicyclic hydrocarbon group is a polycyclic cycloalkyl group, the number of carbon atoms of the alicyclic hydrocarbon group is preferably 6 to 24, more preferably 6 to 20, still more preferably 6 to 18, yet more preferably 6 to 12, and further preferably 7 to 12.

Examples of the aromatic hydrocarbon group include groups obtained by removing nb1 hydrogen atoms of the aryl group. Examples of the aryl group include a phenyl group, a naphthyl group, an anthryl group, a biphenyl group, and a fluorenyl group. More specifically, aromatic hydrocarbon groups represented by the following formulas are exemplified.

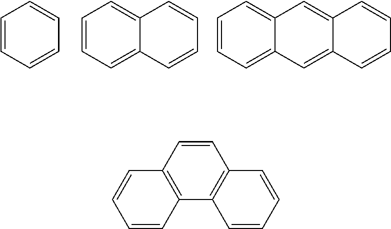

The number of carbon atoms of the aromatic hydrocarbon group is preferably 4 to 24, more preferably 4 to 20, still more preferably 4 to 18, yet more preferably 5 to 14, further preferably 5 to 10, and still further preferably 6 to 10.

Examples of the group obtained by combining two or more groups of the chain hydrocarbon group, the alicyclic hydrocarbon group, and the aromatic hydrocarbon group include a group obtained by combining the chain hydrocarbon group with the alicyclic hydrocarbon group, a group obtained by combining the chain hydrocarbon group with the aromatic hydrocarbon group, a group obtained by combining the alicyclic hydrocarbon group with the aromatic hydrocarbon group, and a group obtained by combining the chain hydrocarbon group, the alicyclic hydrocarbon group, and the aromatic hydrocarbon group. The group obtained by combining the alicyclic hydrocarbon group with the aromatic hydrocarbon group may also be a fused ring.

When —CH₂— included in the hydrocarbon group as for L^{b1} is substituted with —O—, —CO—, —S—, —SO—, or —SO₂—, the number of carbon atoms before substitution is defined as the number of carbon atoms of the hydrocarbon group.

Examples of the group in which —CH₂— included in the chain hydrocarbon group is substituted with —O—, —CO—, —S—, —SO—, or —SO₂—_{among the hydrocarbon groups of L}^{b1} include a hydroxy group, a carboxy group, a carbonyl group, an oxy group, an alkoxy group, an alkoxycarbonyl group, an alkylcarbonyl group, an alkylcarbonyloxy group, an alkoxycarbonyloxy group, an alkanediyloxy group, an alkanediyloxycarbonyl group, an alkanediylcarbonyl group, and an alkanediylcarbonyloxy group. Examples of these substituted groups include those that are the same as described in the present specification as long as the upper limit of the number of carbon atoms permits. The number of carbon atoms in these substituted groups is, for example, 1 to 35, 1 to 17, preferably 1 to 11, more preferably 1 to 6, still more preferably 1 to 4, and still more preferably 1 to 3 in the case of an alkoxy group or the like, 2 to 35, 2 to 17, preferably 2 to 11, more preferably 2 to 6, still more preferably 2 to 4, and still more preferably 2 or 3 in the case of an alkoxycarbonyl group or the like, 2 to 36, 2 to 18, preferably 2 to 12, more preferably 2 to 6, still more preferably 2 to 4, and still more preferably 2 or 3 in the case of an alkylcarbonyl group or the like, and 2 to 34, 2 to 16, preferably 2 to 10, more preferably 2 to 6, and still more preferably 2 to 4, and yet more preferably 2 or 3 in the case of an alkoxycarbonyloxy group or the like.

Examples of the group in which —CH₂— included in the alicyclic hydrocarbon group, the aromatic hydrocarbon group, or the group obtained by combining these groups is substituted with —O—, —CO—, —S—, —SO—, or —SO₂—among the hydrocarbon groups as for L^{b1} include the same groups as those exemplified in the present specification as long as the upper limit of the number of carbon atoms is acceptable.

Examples of the divalent hydrocarbon group as for L^{b2} include a divalent chain hydrocarbon group, a divalent alicyclic hydrocarbon group, a divalent aromatic hydrocarbon group, and groups obtained by combining these two or more groups, and groups that are obtained by removing one hydrogen atom from a monovalent hydrocarbon group, and bonded to Y^{b1}.

Examples of the divalent chain hydrocarbon group, the divalent alicyclic hydrocarbon group, the divalent aromatic hydrocarbon group, and the divalent group obtained by combining these two or more groups as for L^{b2} include groups obtained by removing one hydrogen atom from the monovalent chain hydrocarbon group, the monovalent alicyclic hydrocarbon group, the monovalent aromatic hydrocarbon group, and the monovalent group formed by combining these two or more groups described as for L^{b1}, respectively, as long as the upper limit of the number of carbon atoms is acceptable.

Examples of the substituent that may be possessed by the hydrocarbon group as for L^{b1} and L^{b2} include a halogen atom, a cyano group, and a nitro group. Examples of the halogen atom include a fluorine atom, a chlorine atom, a bromine atom, and an iodine atom.

When L^{b1} and L^{b2} are groups obtained by combining an alicyclic hydrocarbon group or an aromatic hydrocarbon group with a chain hydrocarbon group, the chain hydrocarbon group may be substantially regarded as a substituent that is possessed by the alicyclic hydrocarbon group or the aromatic hydrocarbon group. In addition, —CH₂— of the chain hydrocarbon group included in the hydrocarbon group as for L^{b1} and L^{b2} is substituted with —O—, —CO—, —S—, —SO—, or —SO₂—, whereby the hydrocarbon group as for L^{b1} and L^{b2} can substantially have a substituent such as a hydroxy group, a carboxy group, a carbonyl group, an oxycarbonyl group, a carbonyloxy group, an alkoxy group, an alkylcarbonyl group, an alkoxycarbonyl group, an alkylcarbonyloxy group, a thiol group, an alkylthio group, or an alkylsulfonyl group.

Examples of the cyclic hydrocarbon group as for Y^{b1} include an alicyclic hydrocarbon group, an aromatic hydrocarbon group, and groups obtained by combining an alicyclic hydrocarbon group with an aromatic hydrocarbon group.

Examples of the alicyclic hydrocarbon group and the aromatic hydrocarbon group as for Y^{b1} include same alicyclic hydrocarbon groups and aromatic hydrocarbon groups as described as for L^{b1}, respectively, and when having no substituent, they may be a monovalent alicyclic hydrocarbon group and a monovalent aromatic hydrocarbon group.

Examples of the substituent that may be possessed by the methyl group and the cyclic hydrocarbon group as for Y^{b1} include a halogen atom, a cyano group, a nitro group, or a hydrocarbon group having 1 to 18 carbon atoms and optionally having a halogen atom, a cyano group or a nitro group (—CH₂— included in the hydrocarbon group may be substituted with —O—, —S—, —CO—, —SO—, or —SO₂—). The number of carbon atoms of the hydrocarbon group that may be possessed by the cyclic hydrocarbon group as for Y^{b1} is not included in the number of carbon atoms of the cyclic hydrocarbon group as for Yb1.

Examples of the hydrocarbon group having 1 to 18 carbon atoms, the group that may be possessed, as the substituent, by the methyl group and the cyclic hydrocarbon group as for Y^{b1}, include a chain hydrocarbon group, an alicyclic hydrocarbon group, an aromatic hydrocarbon group, and groups obtained by combining these groups. Examples of the chain hydrocarbon group, the alicyclic hydrocarbon group, the aromatic hydrocarbon group, and groups obtained by combining these two or more groups include the same groups as described in the chain hydrocarbon group, the alicyclic hydrocarbon group, the aromatic hydrocarbon group, and groups obtained by combining these two or more groups as for L^{b1} as long as the upper limit of the number of carbon atoms is acceptable. In addition, examples of the group in which —CH₂— included in the hydrocarbon group having 1 to 18 carbon atoms, the group that may be possessed, as the substituent, by the methyl group and the cyclic hydrocarbon group as for Y^{b1}, is substituted with —O—, —S—, —CO—, —SO—, or —SO₂— include the same groups as described in the group in which —CH₂— included in the hydrocarbon group as for L^{b1} is substituted with —O—, —S—, —CO—, —SO—, or —SO₂— as long as the upper limit of the number of carbon atoms is acceptable. The hydrocarbon group having 1 to 18 carbon atoms, the group that may be possessed, as the substituent, by the cyclic hydrocarbon group as for Y^{b1}, may constitute a protecting group or a leaving group (an acid-labile group or a base-labile group) that is generally used in the relevant field.

The anion of the salt represented by the formula (B1) is preferably an anion represented by the following formula (B1-A1) (hereinafter, may be referred to as “anion (B1-A1)”) or an anion represented by the following formula (B1-A2) (hereinafter, may be referred to as “anion (B1-A2)”).

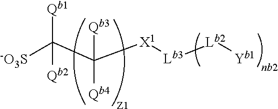

[In the formula (B1-A1),
L^{b2} and Y^{b1} are the same as defined in the formula (B1).

Q^{b1}, Q^{b2}, Q^{b3}, and Q^{b4} each independently represent a hydrogen atom, a fluorine atom, a perfluoroalkyl group having 1 to 6 carbon atoms, or an alkyl group having 1 to 6 carbon atoms.

z1 represents an integer of 0 to 6. When z1 is 2 or more, a plurality of groups in parentheses may be the same as or different from each other.

X¹ represents —O—CO—, —CO—O—, —O—CO—O—, or —O—.

L^{b3} represents single bond or an (nb2+1)-valent hydrocarbon group having 1 to 24 carbon atoms and optionally having a substituent, and —CH₂— included in the hydrocarbon group may be substituted with —O—, —S—, —CO, —SO—, or —SO₂—.

nb2 represents an integer of 1 to 3. When nb2 is 2 or more, a plurality of groups in parentheses may be the same as or different from each other.]

Examples of the alkyl group as for Q^{b1}, Q^{b2}, Q^{b3}, and Q^{b4} include a methyl group, an ethyl group, a propyl group, an isopropyl group, a butyl group, a sec-butyl group, a tert-butyl group, a pentyl group, and a hexyl group.

Examples of the perfluoroalkyl group as for Q^{b1}, Q^{b2}, Q^{b3}, and Q^{b4} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, and a perfluorohexyl group.

Q^{b1} and Q^{b2} preferably include a fluorine atom or a perfluoroalkyl group in at least one of Q^{b1} and Q^{b2}, more preferably a fluorine atom or a perfluoroalkyl group, still more preferably a fluorine atom or a trifluoromethyl group, and yet more preferably both are fluorine atoms.

Preferably, Q^{b3} and Q^{b4} are each independently a hydrogen atom, a fluorine atom, or a perfluoroalkyl group having 1 to 3 carbon atoms, Q^{b3} is preferably a hydrogen atom, a fluorine atom, or a perfluoroalkyl group having 1 to 3 carbon atoms, and Q^{b4} is preferably a hydrogen atom or a fluorine atom.

z1 is preferably an integer of 0 to 3, and more preferably 0, 1 or 2.

X¹ is preferably —O—CO— or —CO—O—.

Examples of the hydrocarbon group in L^{b3} include the same hydrocarbon groups as described as for L^{b1} of the formula (B1) as long as the upper limit of the number of carbon atoms is acceptable.

L^{b3} is preferably a single bond, a chain hydrocarbon group having 1 to 12 carbon atoms and optionally having a substituent (—CH₂— included in the chain hydrocarbon group may be substituted with —O— or —CO—), an alicyclic hydrocarbon group having 3 to 18 carbon atoms and optionally having a substituent (—CH₂— included in the alicyclic hydrocarbon group may be substituted with —O—, —S—, —CO—, —SO—, or —SO₂—), an aromatic hydrocarbon group having 6 to 10 carbon atoms and optionally having a substituent (—CH₂— included in the aromatic hydrocarbon group may be substituted with —O— or —S—) or groups obtained by combining these two or more groups, and more preferably a single bond, a chain hydrocarbon group having 1 to 6 carbon atoms, or a group represented by the following formula (Lb3-1). When —CH₂— included in the chain hydrocarbon group is substituted with —O— or —CO—, the number of —CH₂— included in the chain hydrocarbon group substituted with —O— or —CO— is preferably 1 to 4, and one —CH₂—CH₂— included in the chain hydrocarbon group is preferably substituted with —O— CO— or —CO—O—, or one —CH₂—CH₂—CH₂— included in the chain hydrocarbon group is preferably substituted with —O—CO—O—.

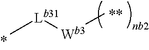

[In the formula (Lb3-1),
nb2 is the same as defined in the formula (B1-A1).

L^{b31} represents a single bond or a chain hydrocarbon group having 1 to 12 carbon atoms, —CH₂— included in the chain hydrocarbon group may be substituted with —O— or —CO—, and the chain hydrocarbon group may have a substituent.

W^{b3} represents an alicyclic hydrocarbon group having 3 to 18 carbon atoms or an aromatic hydrocarbon group having 6 to 10 carbon atoms, —CH₂— included in the alicyclic hydrocarbon group may be substituted with —O—, —S—, —CO—, —SO—, —SO₂—, or, —CH₂— included in the aromatic hydrocarbon group may be substituted with —O— or —S—, and the alicyclic hydrocarbon group and the aromatic hydrocarbon group may have a substituent.

* and ** represent a binding site, and * represents a binding site to X¹.

In the formula (Lb3-1), examples of the chain hydrocarbon group as for L^{b31} include the same chain hydrocarbon groups as described as for L^{b1} as long as the upper limit of the number of carbon atoms is acceptable.

In the formula (Lb3-1), examples of the alicyclic hydrocarbon group and the aromatic hydrocarbon group as for W^{b3} include the same alicyclic hydrocarbon groups and aromatic hydrocarbon groups as described as for L^{b1} as long as the upper limit of the number of carbon atoms is acceptable.

In the formula (Lb3-1), examples of the substituent that may be possessed by the chain hydrocarbon group as for L^{b31} and the substituent that may be possessed by the alicyclic hydrocarbon group and the aromatic hydrocarbon group as for W^{b3} include the same substituents as described as for the substituent that may be possessed by the hydrocarbon group as for L^{b1}.

L^{b31} is preferably a single bond or an alkanediyl group having 1 to 6 carbon atoms (—CH₂— included in the alkanediyl group may be substituted with —O— or —CO—).

Examples of the alicyclic hydrocarbon group and the aromatic hydrocarbon group as for W^{b3} include an alicyclic hydrocarbon group and an aromatic hydrocarbon group represented below. In the alicyclic hydrocarbon group and the aromatic hydrocarbon group described below, * and ** represent a binding site, * represents a binding site to X¹ or L^{b31}, ** represents a binding site to a hydrogen atom, a substituent or L^{b2}, and at least one ** represents a binding site to L^{b2}. In addition, in the alicyclic hydrocarbon group described below, —CH₂— included in the alicyclic hydrocarbon group may be substituted with —O—, —S—, —CO—, —SO—, or —SO₂—. When —CH₂— included in the alicyclic hydrocarbon group is substituted with —O—, —S—, —CO—, or —SO₂—, it is preferable to form an ether ring, an ester ring (lactone), a carbonic acid ester ring, a sulfonic acid ester ring (sultone), or an acetal ring.

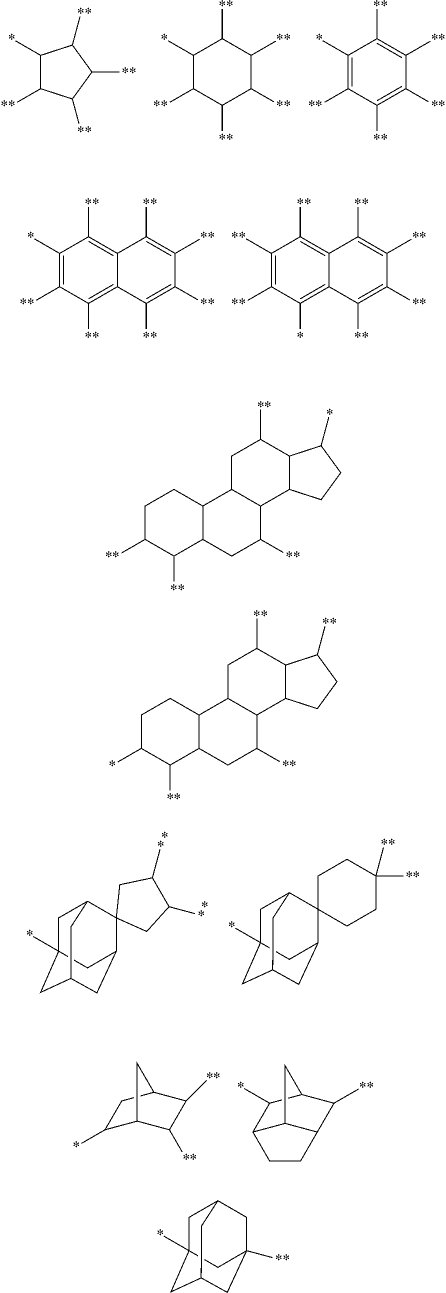

In the formula (B1-A1), L^{b2} is preferably a single bond or a chain hydrocarbon group having 1 to 12 carbon atoms (—CH₂— included in the chain hydrocarbon group may be substituted with —O— or —CO—), and more preferably a single bond, —O—, —O—CO—, —CO—O—, —O—CO—O—, or *-L^{b21}-X²-L^{b22}-** (one of L^{b21} and L^{b22} represents a chain hydrocarbon group having 1 to 6 carbon atoms, and the other one represents a single bond or a chain hydrocarbon group having 1 to 6 carbon atoms, X² represents —O—, —CO—O—, —O—CO—, or —O—CO—O—, * and ** represent a binding site, and ** represents a binding site to Y^{b1}, in which the total number of carbon atoms of L^{b2}, X² and L^{b22} is 12 or less).

In the formula (B1-A1), Yb¹ is preferably a cyclic hydrocarbon group having 3 to 20 carbon atoms and optionally having a substituent (—CH₂— included in the cyclic hydrocarbon group may be substituted with —O—, —CO—, —S—, —SO—, or —SO₂—), more preferably, an alicyclic hydrocarbon group having 3 to 18 carbon atoms and optionally having a substituent (—CH₂— included in the alicyclic hydrocarbon group may be substituted with —O—, —CO—, —S—, —SO—, or —SO₂—) or an aromatic hydrocarbon group having 6 to 18 carbon atoms and optionally having a substituent, and still more preferably an alicyclic hydrocarbon group having 3 to 16 carbon atoms and optionally having a substituent (—CH₂— included in the alicyclic hydrocarbon group may be substituted with —O—, —CO—, —S—, —SO—, or —SO₂—) or an aromatic hydrocarbon group having 6 to 10 carbon atoms and optionally having a substituent. Specifically, groups represented by the following formula (Y1) to formula (Y36) are more preferable. In the formula (Y1) to the formula (Y36), RYb represents a hydrogen atom or an alkyl group having 1 to 4 carbon atoms among substituents that may be possessed by the cyclic hydrocarbon group as for Y^{b1}, R^{Yc} represents a hydrogen atom or a substituent that may be possessed by the cyclic hydrocarbon group as for Y^{b1}, and * represents a binding site to L^{b2}. The alicyclic hydrocarbon group and the aromatic hydrocarbon group represented by the following formulas are not particularly shown in the following formulas, but may have any other substituents.

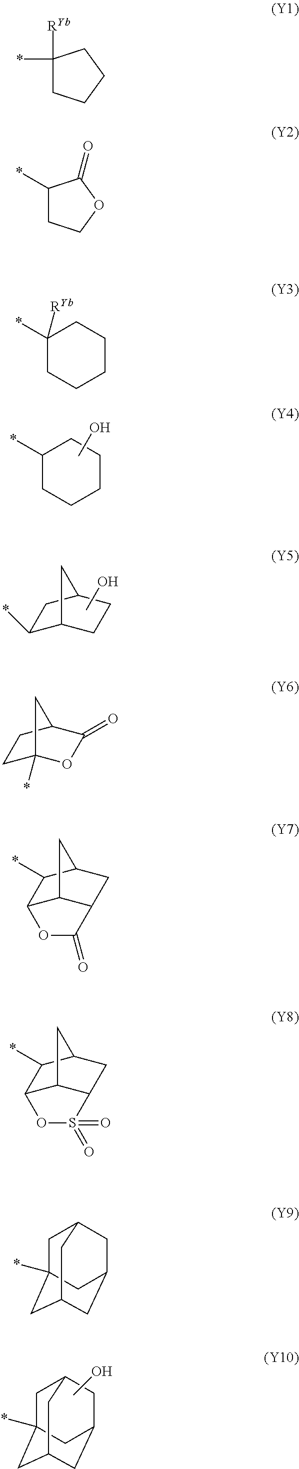

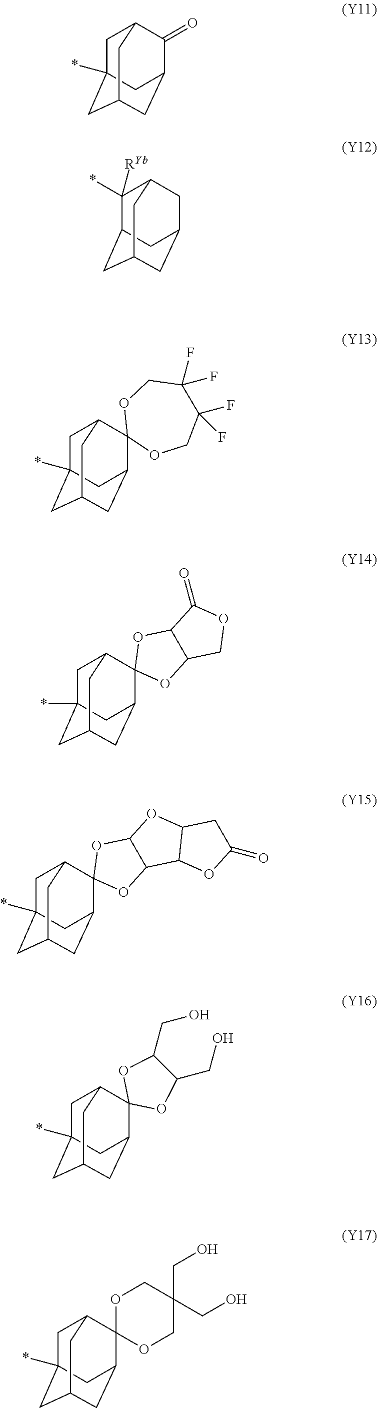

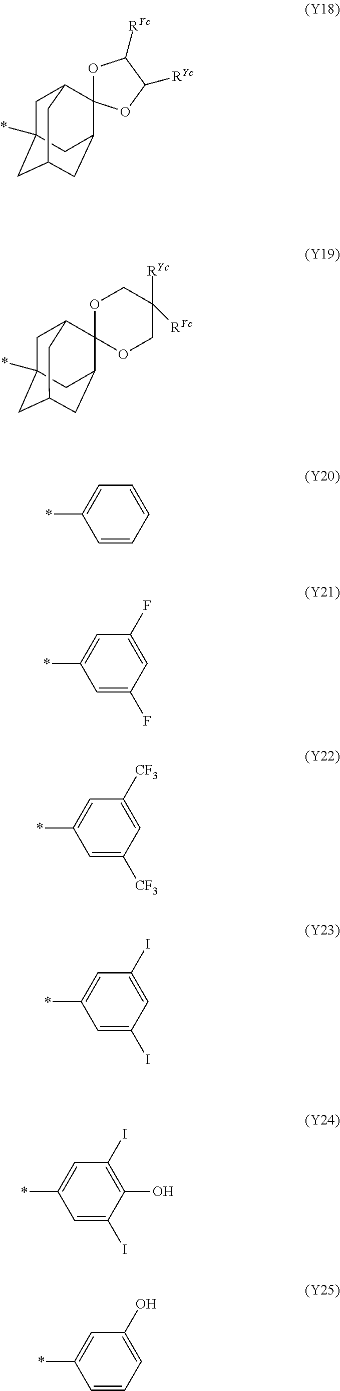

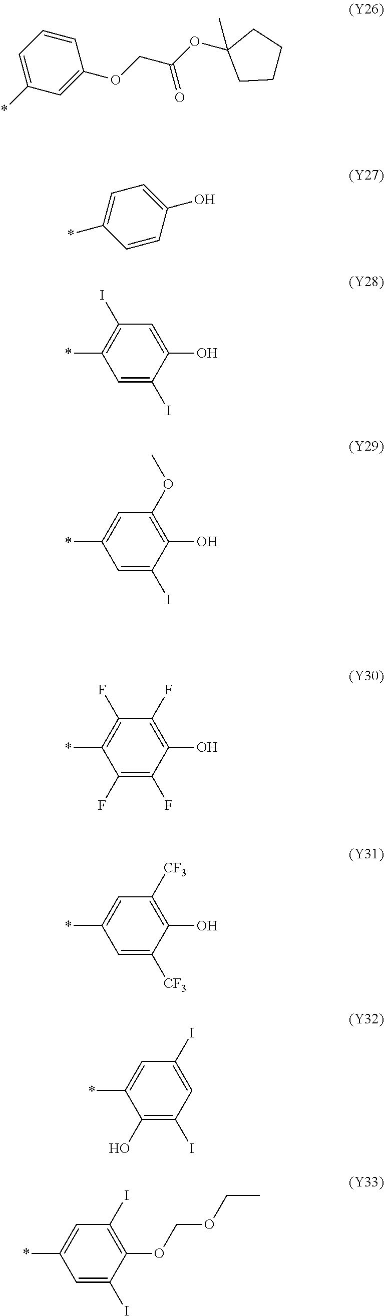

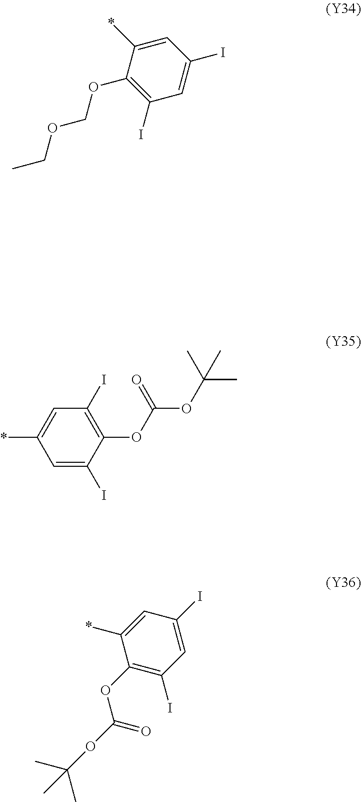

The anion represented by the formula (B1-A1) is preferably an anion represented by a formula (B1-A1-1) to a formula (B1-A1-85) [hereinafter, may be referred to as “anion (B1-A1-1)” or the like according to the formula number], and more preferably an anion represented by any one of the formula (B1-A1-1) to a formula (B1-A1-4), a formula (B1-A1-9), a formula (B1-A1-10), a formula (B1-A1-24) to a formula (B1-A1-33), a formula (B1-A1-36) to a formula (B1-A1-40), and a formula (B1-A1-47) to a formula (B1-A1-85).

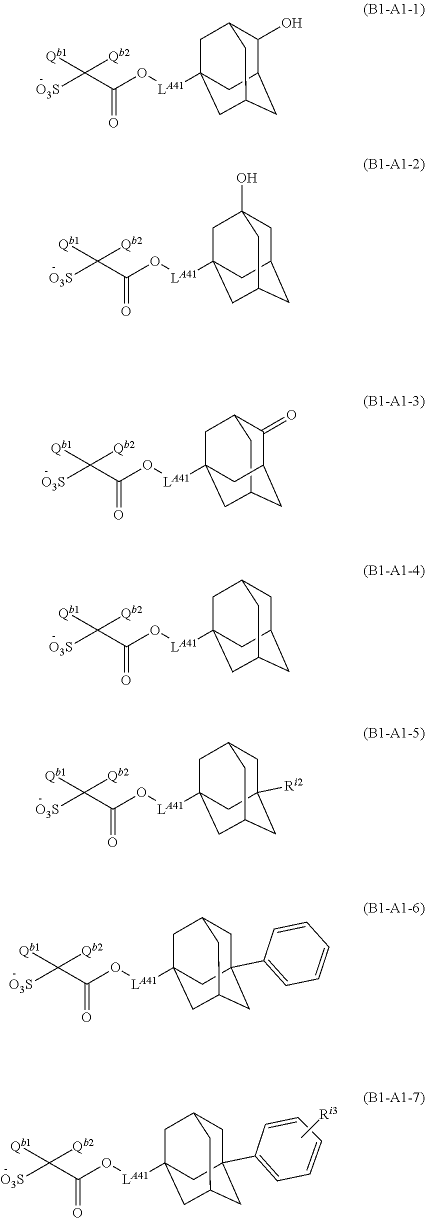

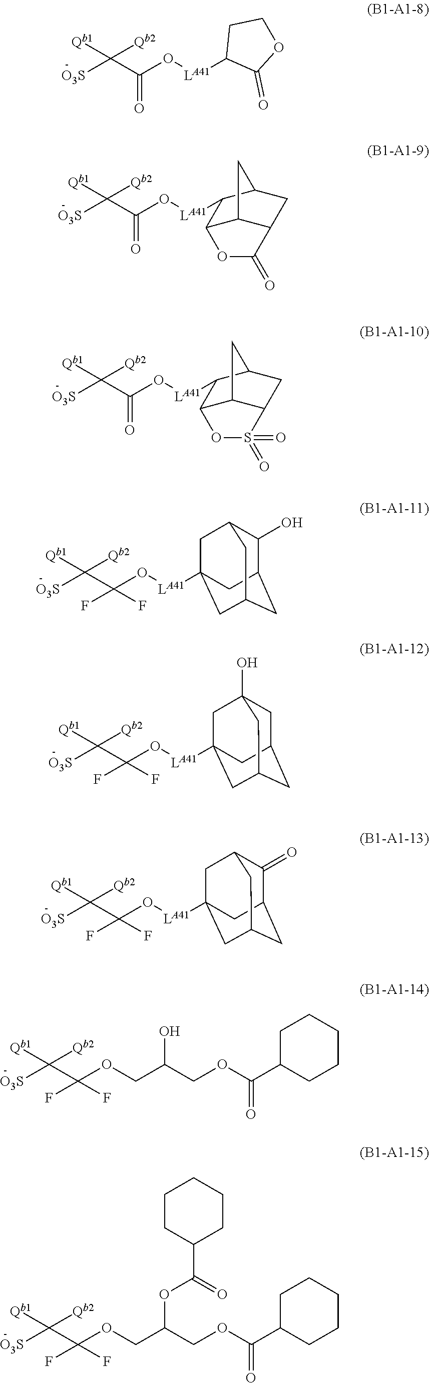

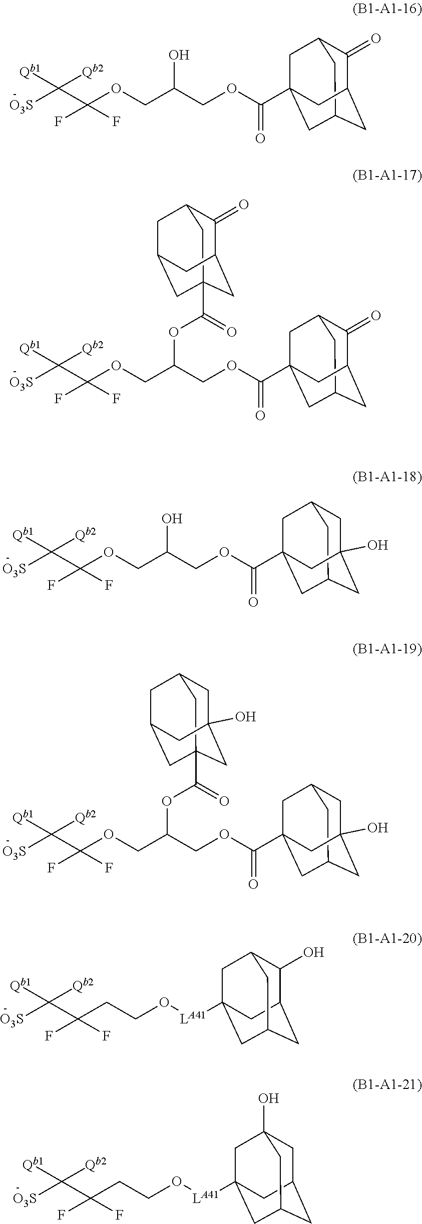

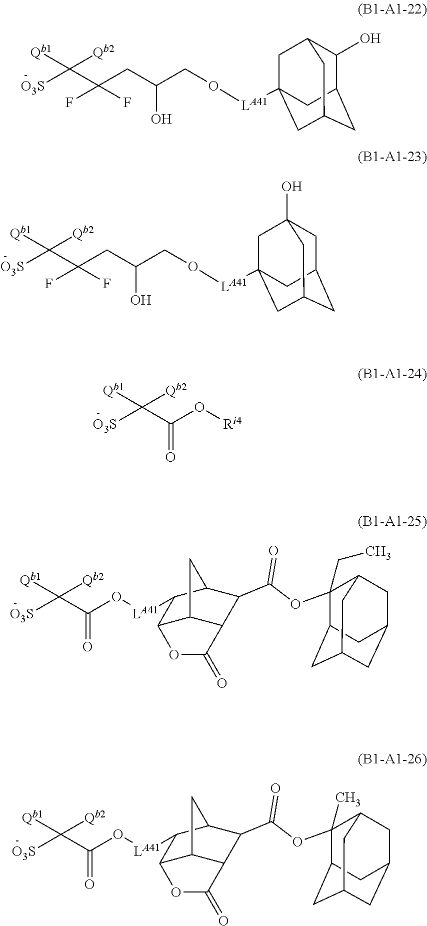

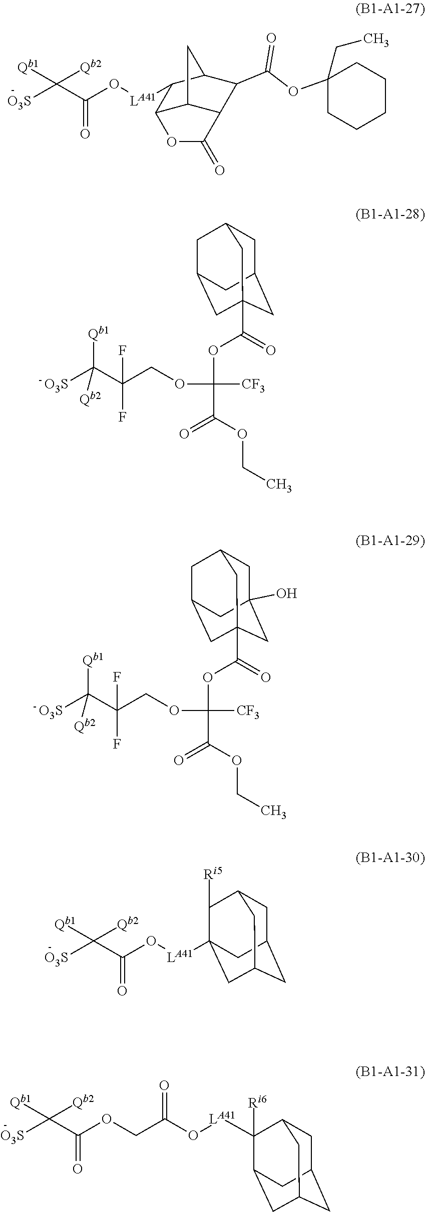

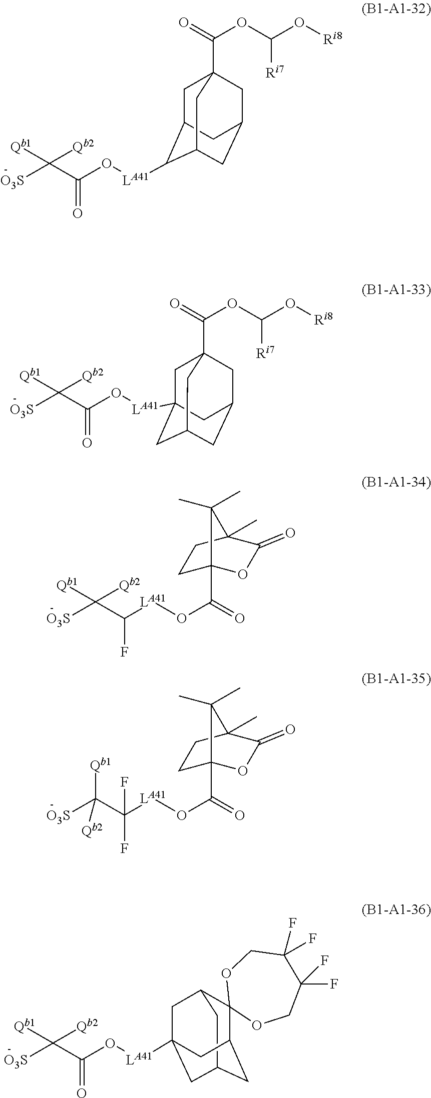

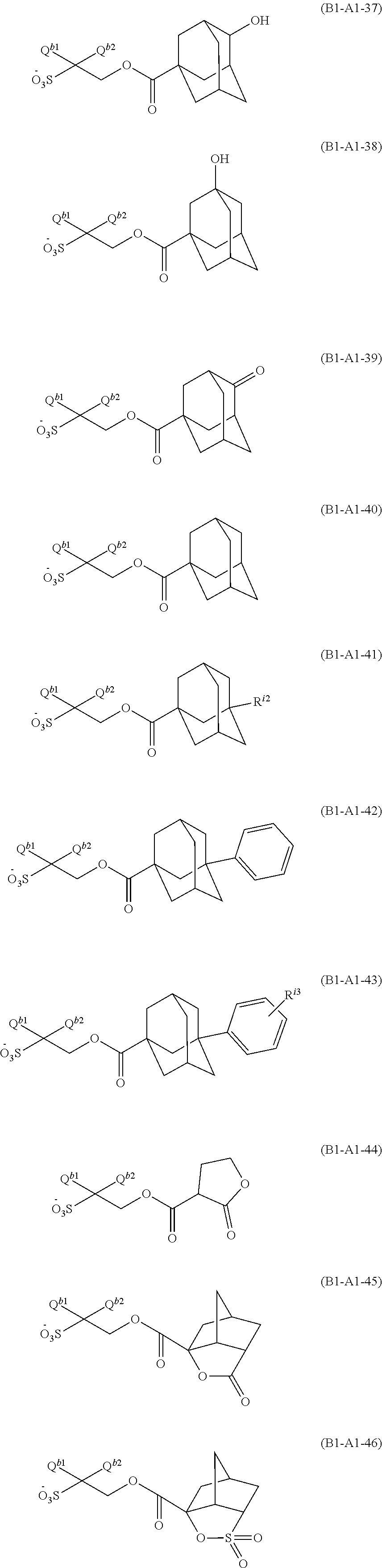

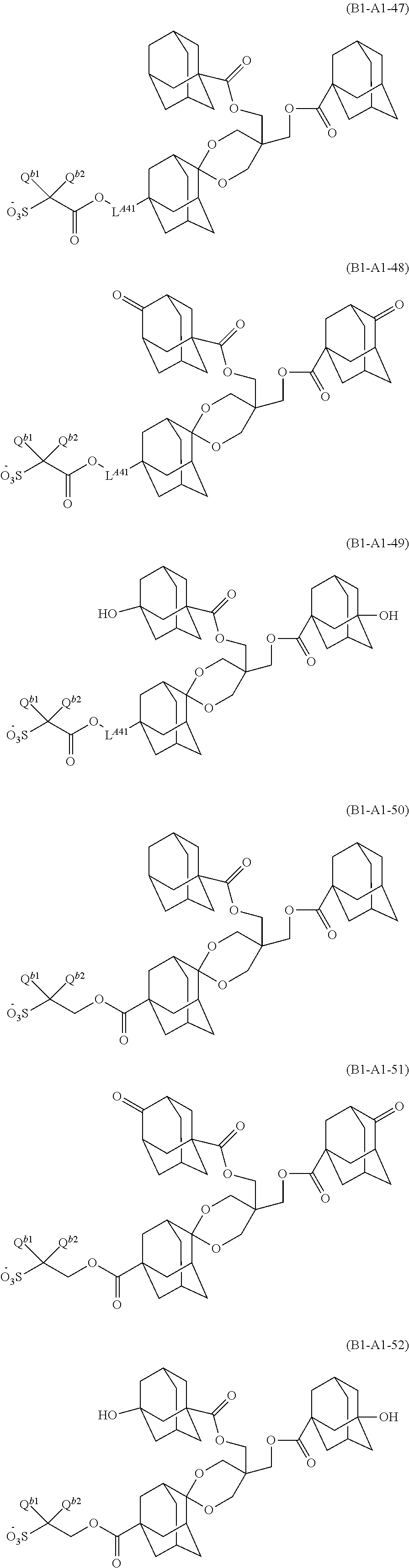

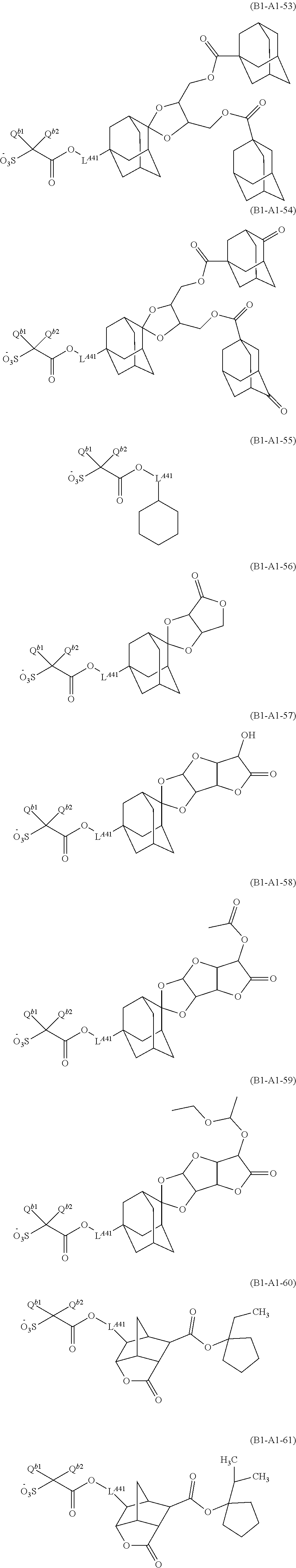

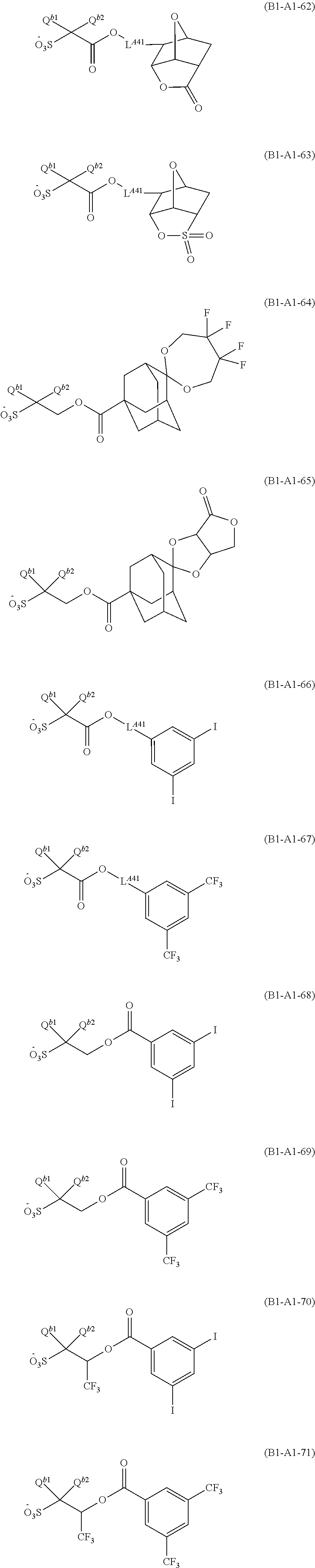

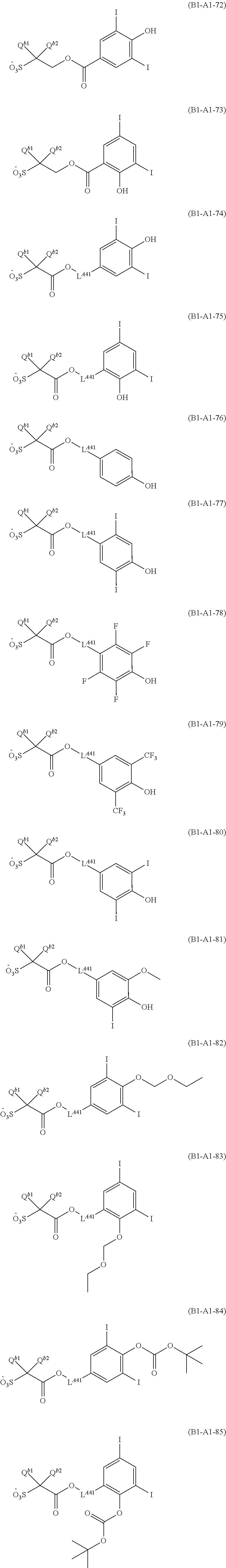

Herein, Rⁱ² to Rⁱ⁷ are each independently, for example, an alkyl group having 1 to 4 carbon atoms, and preferably a methyl group or an ethyl group. Rⁱ⁸ is, for example an aliphatic hydrocarbon group having 1 to 12 carbon atoms, preferably an alkyl group having 1 to 4 carbon atoms, an alicyclic hydrocarbon group having 5 to 12 carbon atoms, or a group formed by combining these groups, and more preferably a methyl group, an ethyl group, a cyclohexyl group, or an adamantyl group. L^{A41} is a single bond or an alkanediyl group having 1 to 4 carbon atoms.

Q^{b1} and Q^{b2} are the same as defined above.

Specific examples of the anion represented by the formula (B1-A1) include anions described in JP-A-2010-204646.

Examples of preferred anions represented by the formula (B1-A1) include anions represented by a formula (B1a-1) to a formula (B1a-82). Among these, an anion represented by any one of the formula (B1a-1) to a formula (B1a-4), a formula (B1a-7) to a formula (B1a-11), a formula (B1a-14) to a formula (B1a-30), and a formula (B1a-35) to a formula (B1a-70) is preferable.

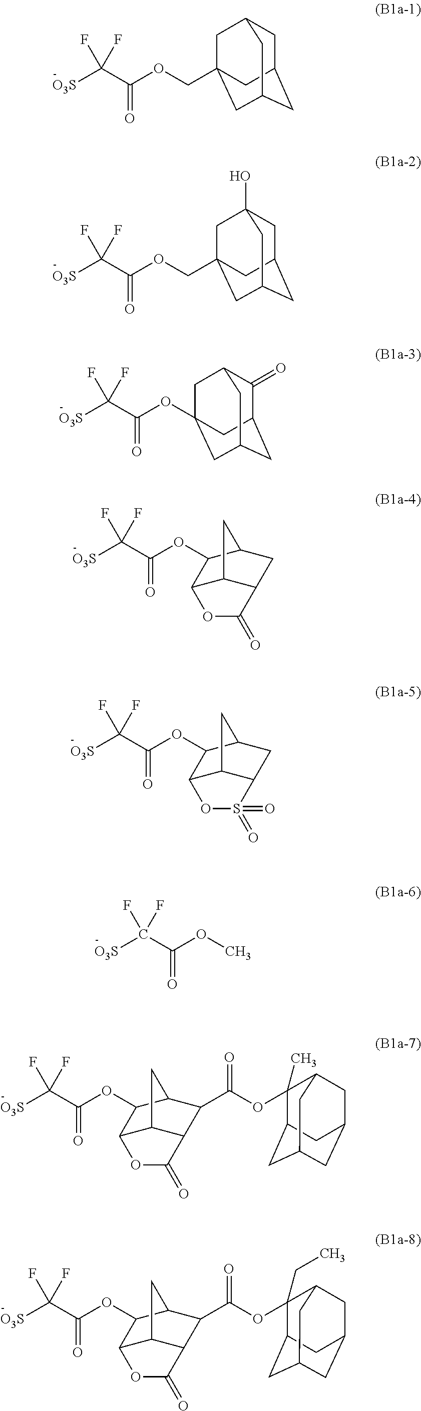

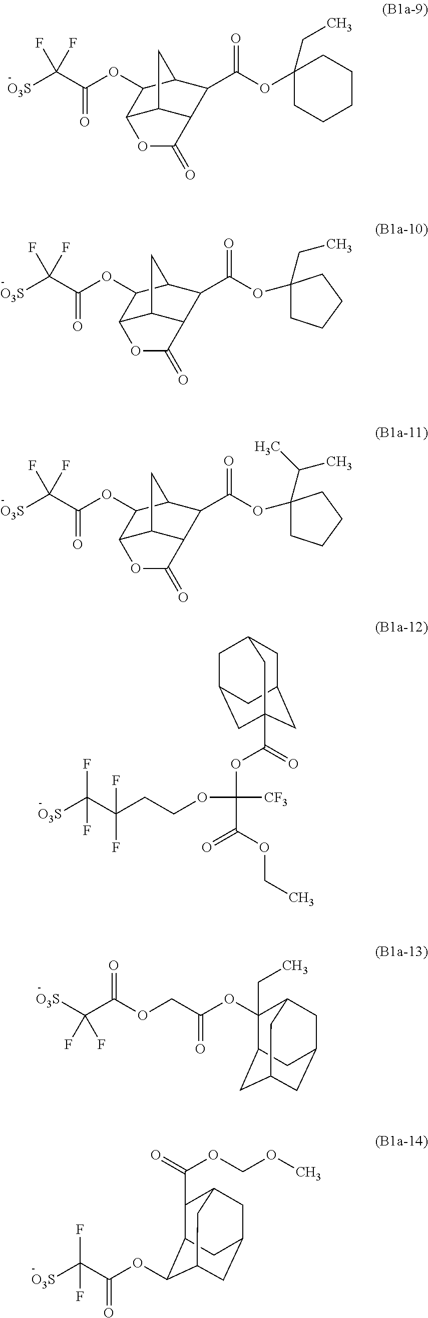

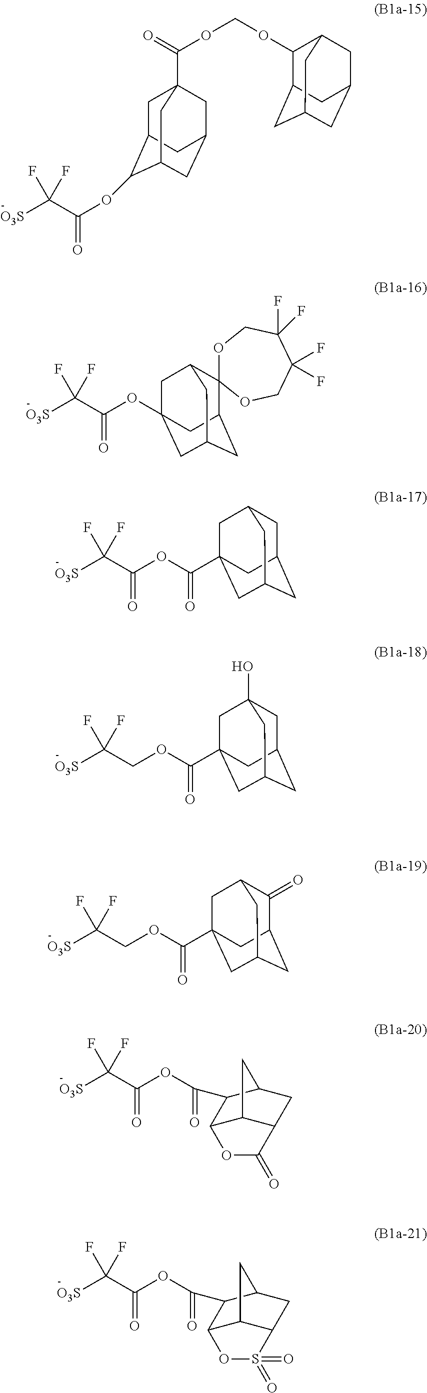

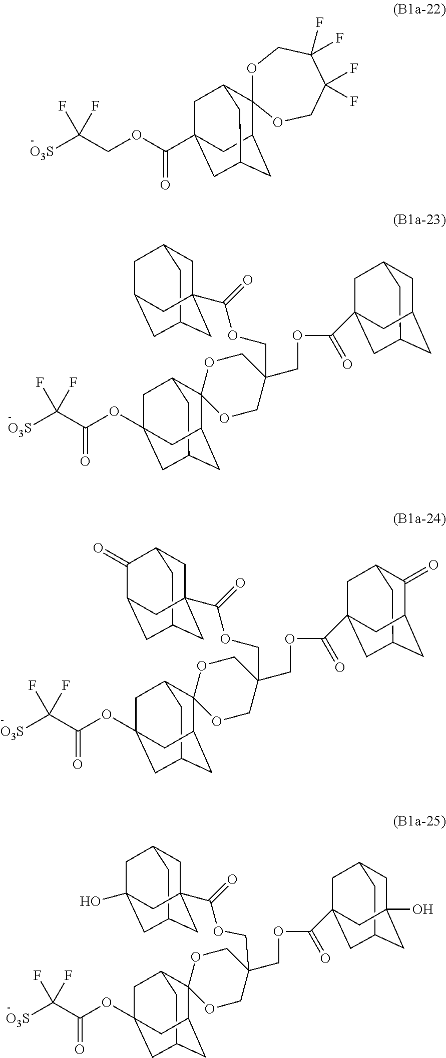

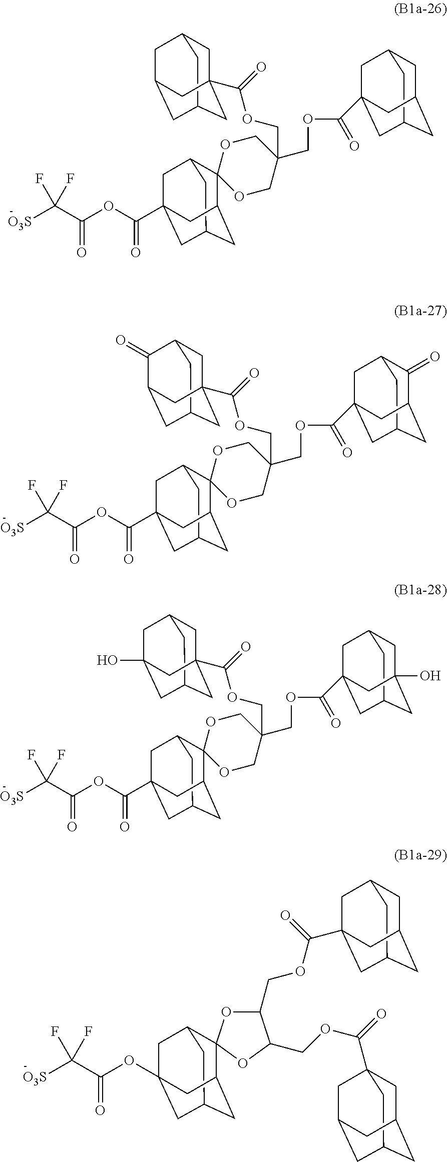

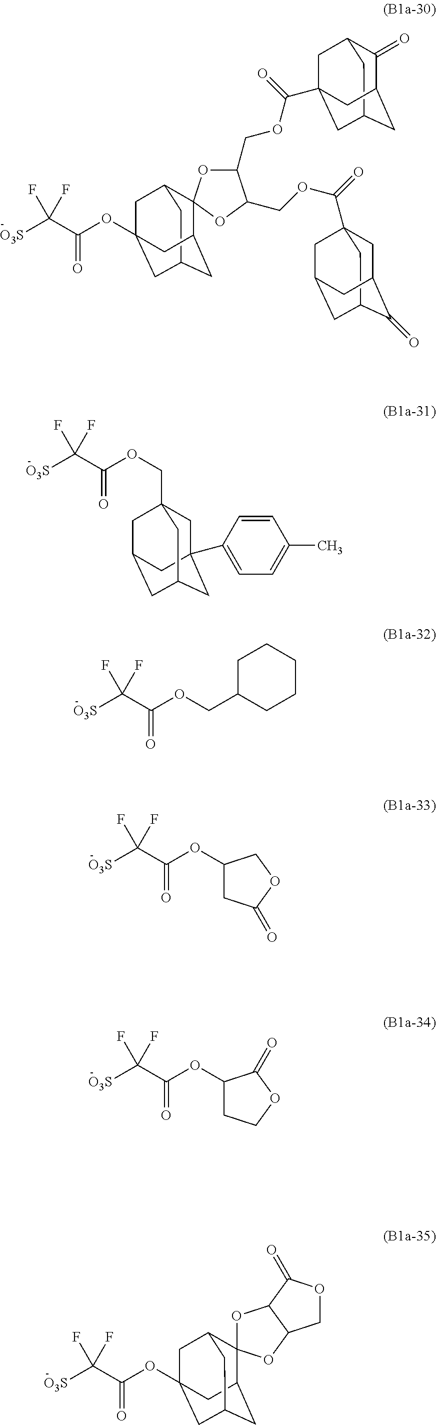

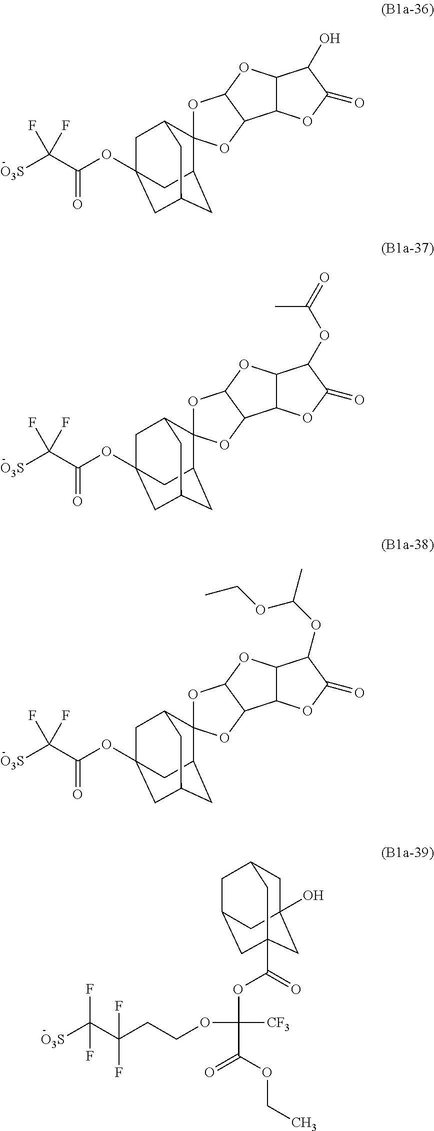

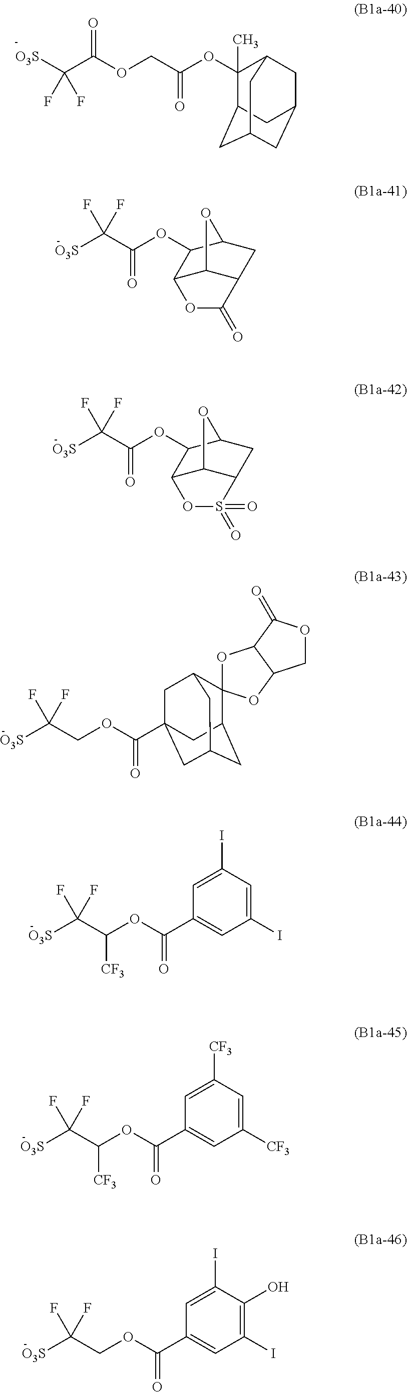

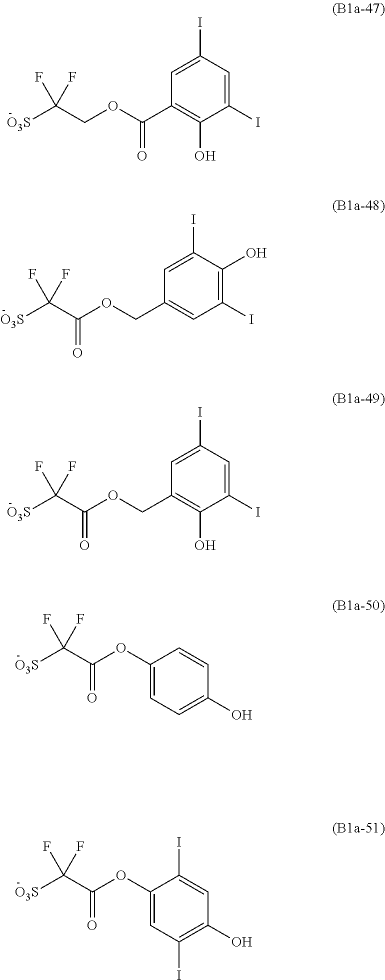

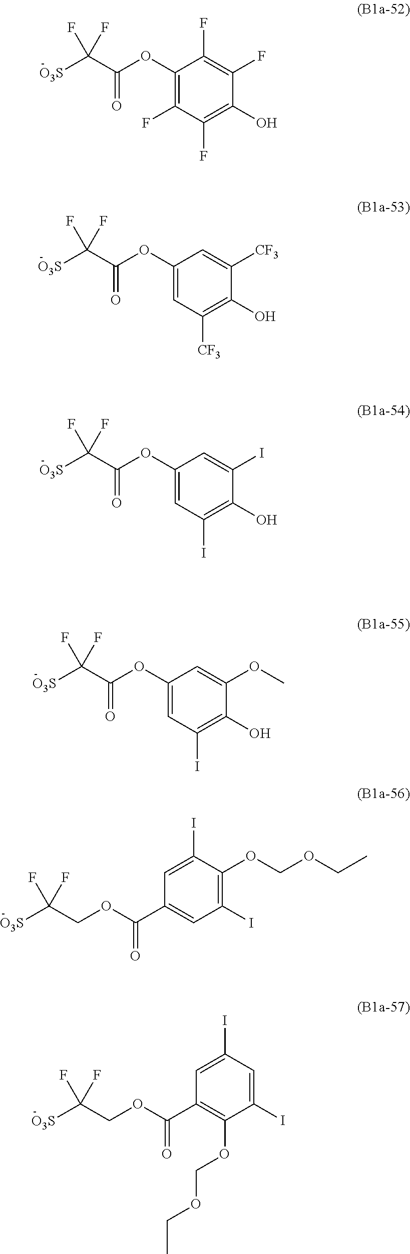

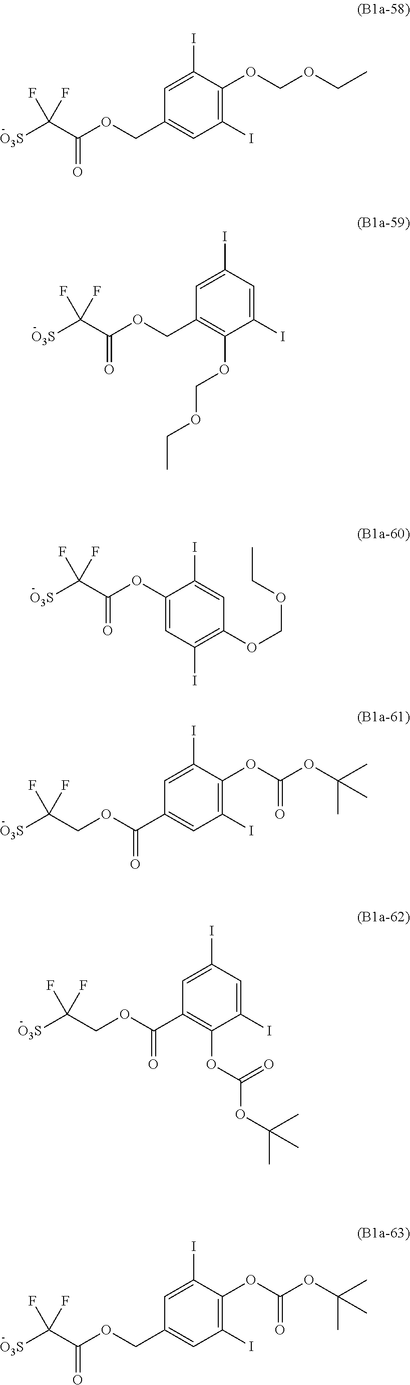

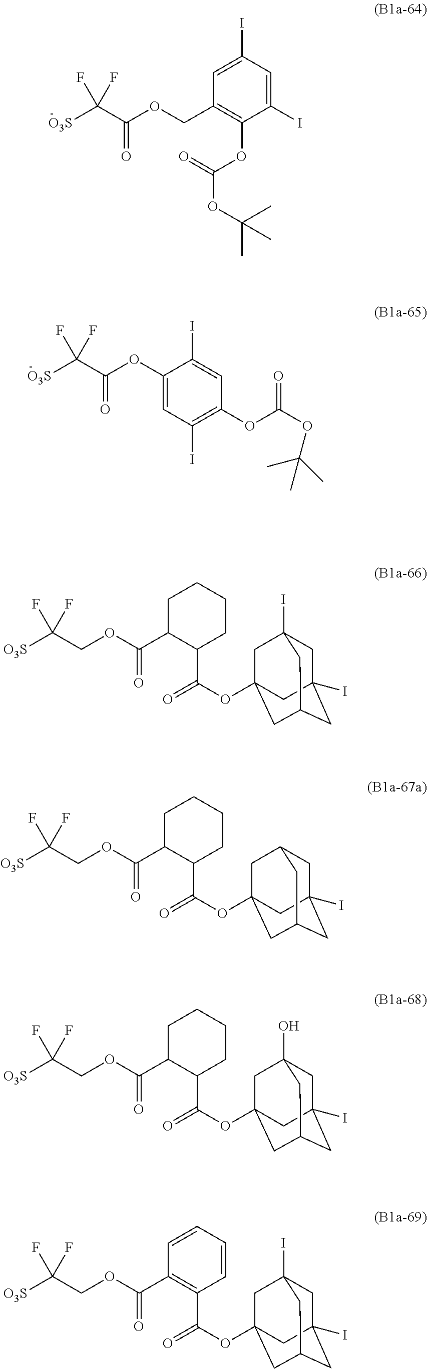

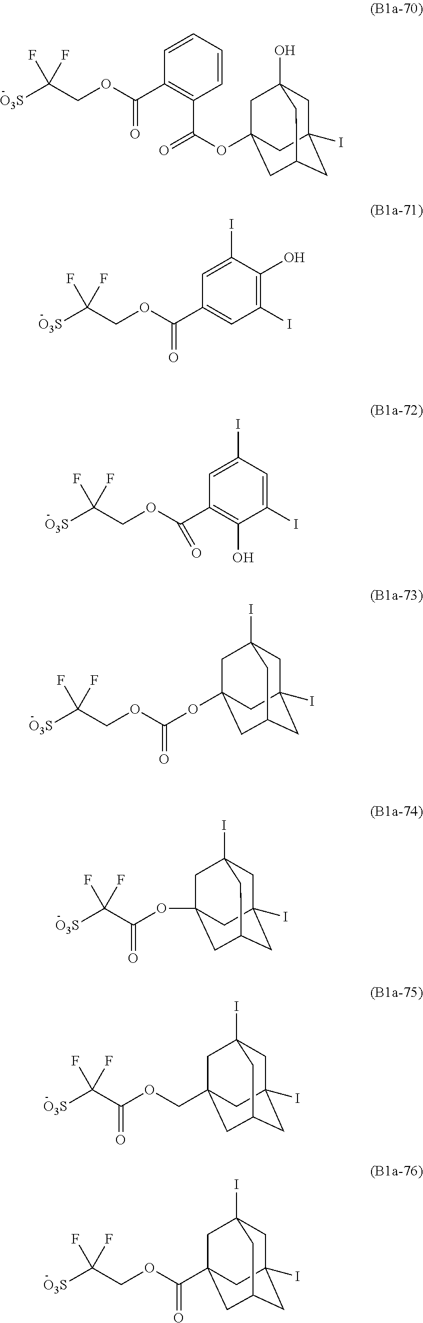

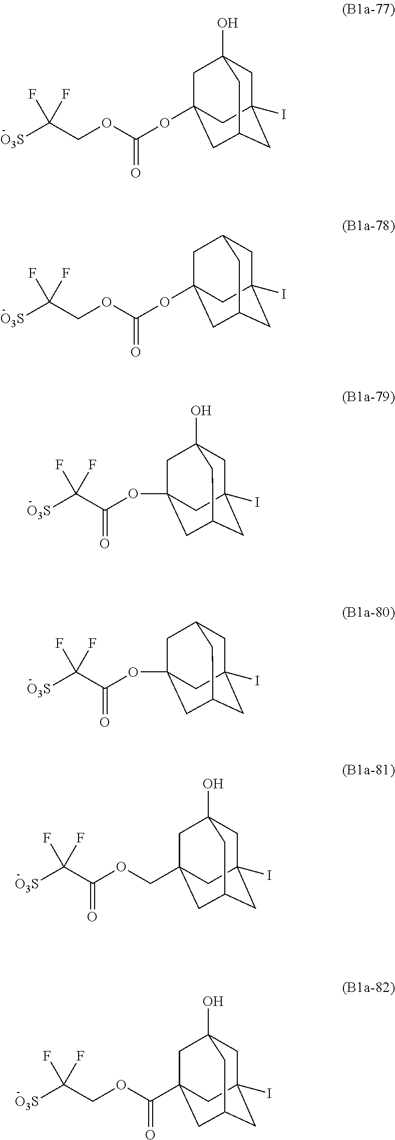

The anion represented by the formula (B1-A2) is represented by the following formula.

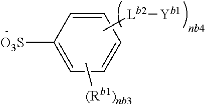

[In the formula (B1-A2),
L^{b2} and Y^{b1} are the same as defined in the formula (B1).

R^{b1} represents a halogen atom or an alkyl group having 1 to 6 carbon atoms, and —CH₂— included in the alkyl group may be substituted with —O— or —CO—.

nb4 represents an integer of 1 to 5. When nb4 is 2 or more, a plurality of groups in parentheses may be the same as or different from each other.

nb3 represents an integer of 0 to 4. When nb3 is 2 or more, a plurality of R^{b1} may be the same as or different from each other.

Provided that 1≤nb4+nb3≤5 is satisfied]

In the formula (B1-A2), examples of the hydrocarbon group as for L^{b2} and examples of the cyclic hydrocarbon group as for Y^{b1} include the same hydrocarbon groups as for L^{b2} and the same cyclic hydrocarbon groups as for Yb¹ in formula (B1) as long as the upper limit of the number of carbon atoms is acceptable. Examples of the substituent that may be possessed by the hydrocarbon group as for L^{b2}, and the methyl group and the cyclic hydrocarbon group as for Y^{b1} also include the same substituents that may be possessed by the hydrocarbon groups as for L^{b2}, and the methyl group and the cyclic hydrocarbon group as for Y^{b1} of the formula (B1).

These hydrocarbon groups, methyl groups, and cyclic hydrocarbon groups may have one substituent or a plurality of substituents. L^{b2} is preferably a chain hydrocarbon group having 1 to 12 carbon atoms (—CH₂— included in the chain hydrocarbon group may be substituted with —O— or —CO—), more preferably *—CO—O-L^{b41}- (L^{b41} is a single bond or a chain hydrocarbon group having 1 to 6 carbon atoms, —CH₂— included in the chain hydrocarbon group may be substituted with —O— or —CO—, * represents a binding site to the benzene ring to which SO₃⁻ is bonded, and L^{b41} is preferably a single bond or a chain hydrocarbon group having 1 to 3 carbon atoms), and still more preferably *—CO—O—.

Y^{b1} is preferably a cyclic hydrocarbon group having 3 to 20 carbon atoms and optionally having a substituent (—CH₂— included in the cyclic hydrocarbon group may be substituted with —O—, —CO—, —S—, —SO—, or —SO₂—), more preferably an alicyclic hydrocarbon group having 3 to 18 carbon atoms and optionally having a substituent (—CH₂— included in the alicyclic hydrocarbon group may be substituted with —O—, —CO—, —S—, —SO—, or —SO₂—) or an aromatic hydrocarbon group having 6 to 18 carbon atoms and optionally having a substituent, and still more preferably an alicycic hydrocarbon group or an aromatic hydrocarbon group exemplified as for Y^{b1} or L^{b1} of the formula (B1-A1). Specifically, the group represented by the formula (Y1) to the formula (Y36) described above is preferable, and the group represented by the formula (Y1) to the formula (Y19) described above is more preferable.

Examples of the substituents that may be possessed by the cyclic hydrocarbon group as for Y^{b1} are preferably a halogen atom or an alkyl group having 1 to 6 carbon atoms (—CH₂— included in the alkyl group may be substituted with —O—, —S—, —CO—, —SO—, or —SO₂—), more preferably a fluorine atom, an iodine atom or an alkyl group having 1 to 4 carbon atoms (—CH₂— included in the alkyl group may be substituted with —O—, —S—, —CO—, —SO—, or —SO₂—), still more preferably a fluorine atom, an iodine atom, a hydroxy group, an alkoxy group having 1 to 3 carbon atoms, or an alkyl group having 1 to 4 carbon atoms, and yet more preferably a fluorine atom, an iodine atom, a hydroxy group or an alkyl group having 1 to 4 carbon atoms. In addition, as the substituent of Y^{b2}, a protecting group or a leaving group (an acid-labile group or a base-labile group) generally used in the art may be constituted.

In the formula (B1-A2), nb4 is preferably an integer of 1 to 4, more preferably an integer of 1 to 3, still more preferably 1 or 2, and yet more preferably 2. When nb4 is 1 or 2, the binding site of -L^{b2}-Y^{b1} is preferably the in-position of the benzene ring, with respect to the binding site of SO₃—, as shown in the following structures.

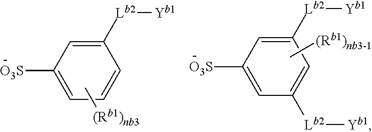

[In the above formulas, L^{b2}, y^{b1}, R^{b1}, and nb3 are the same as defined in the formula (B1-A2).]

In the formula (B1-A2), when nb4 is 2 or more, the plurality of L^{b2} and Y^{b1} are preferably the same group.

Examples of the alkyl group having 1 to 6 carbon atoms as for R^{b1} include alkyl groups such as a methyl group, an ethyl group, a propyl group, an isopropyl group, a butyl group, an isobutyl group, a tert-butyl group, a pentyl group, and a hexyl group. The number of carbon atoms of the alkyl group is preferably 1 to 4, and more preferably 1 to 3.

Examples of the halogen atom as for R^{b1} include a fluorine atom, a chlorine atom, a bromine atom, and an iodine atom.

Examples of the group in which —CH₂— included in the alkyl group is substituted with —O— or —CO— include a hydroxy group, a carboxy group, an alkoxy group, an alkoxycarbonyl group, an alkylcarbonyl group, an alkylcarbonyloxy group, an oxy group, and a carbonyl group. Examples of these substituted groups include those that are the same as described in the present specification as long as the upper limit of the number of carbon atoms permits.

Preferably, R^{b1} is a halogen atom or an alkyl group having 1 to 4 carbon atoms (—CH₂— included in the alkyl group may be substituted with —O— or —CO—), more preferably a fluorine atom, an iodine atom or an alkyl group having 1 to 3 carbon atoms (—CH₂— included in the alkyl group may be substituted with —O— or —CO—), and still more preferably a fluorine atom, an iodine atom, a hydroxy group, a methoxy group or a methyl group.

nb3 is an integer of 0 to 4 (that is, nb3-1 is an integer of 0 to 3), preferably an integer of 0 to 3, and more preferably an integer of 0 to 2. In one embodiment, nb3 is preferably 0. In another embodiment, nb3 is preferably 1 or 2. When nb3 is 1, R^{b1} is preferably a halogen atom, and R^{b1} is more preferably a fluorine atom or an iodine atom. When nb3 is 2, it is preferable that one of R^{b1} is a halogen atom, and the other one is a halogen atom or an alkyl group having 1 to 4 carbon atoms, and it is more preferable that one of R^{b1} is a fluorine atom or an iodine atom, and the other one is a fluorine atom, an iodine atom or an alkyl group having 1 to 3 carbon atoms.

Examples of the anion represented by the formula (B1-A2) include the following anions. Among these, anions represented by a formula (B2a-1) to a formula (B2a-32) are preferable, and anions represented by a formula (B2a-1) to a formula (B2a-11) and a formula (B2a-16) to a formula (B2a-20) are more preferable. The following anions may have a substituent not shown, with a part of R^{b1} being omitted.

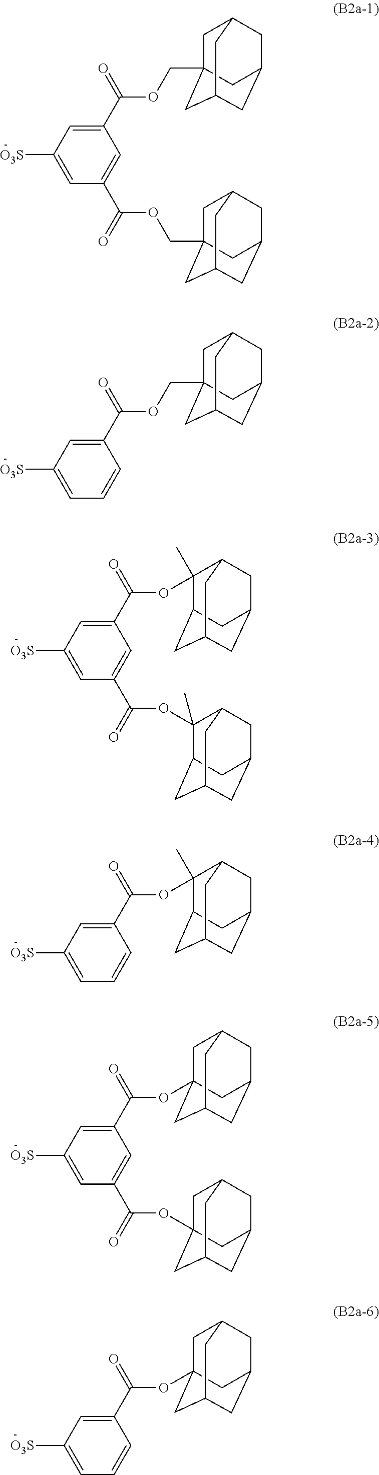

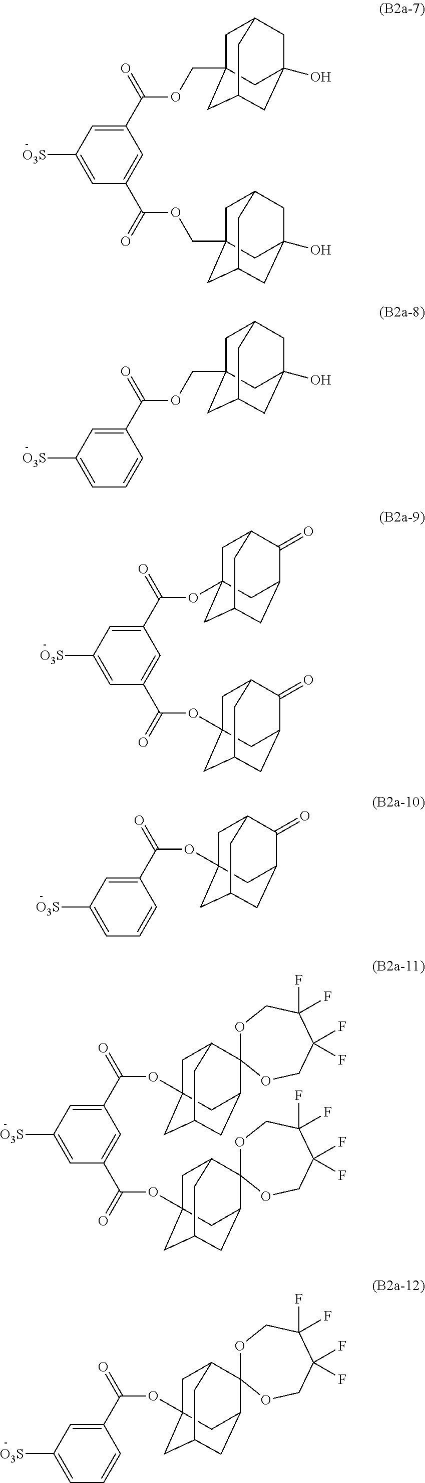

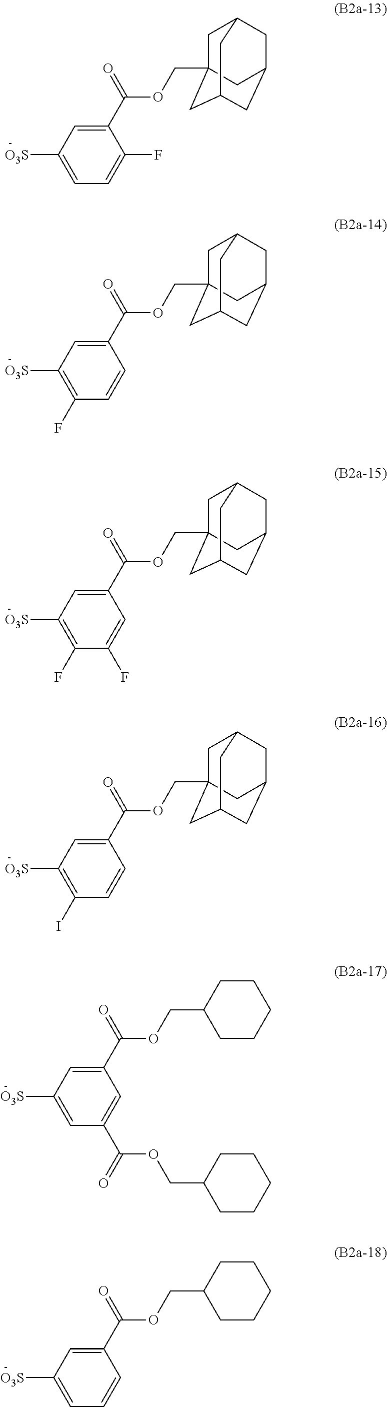

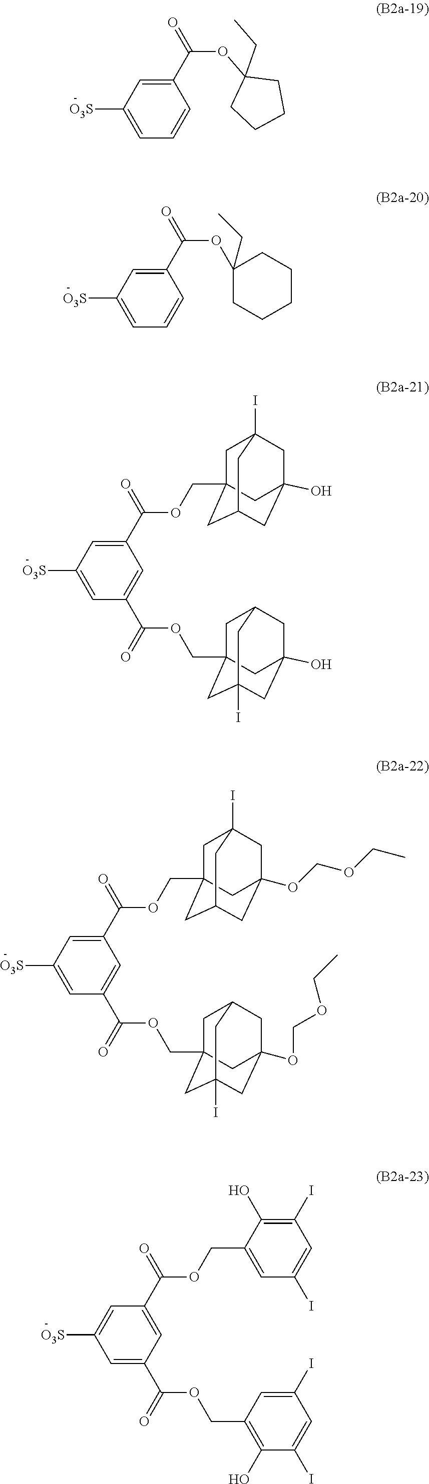

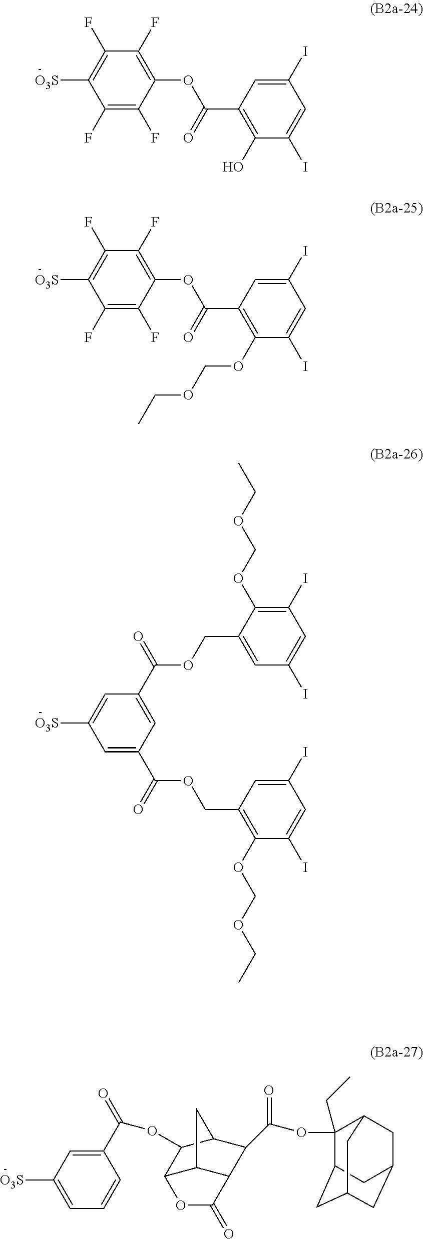

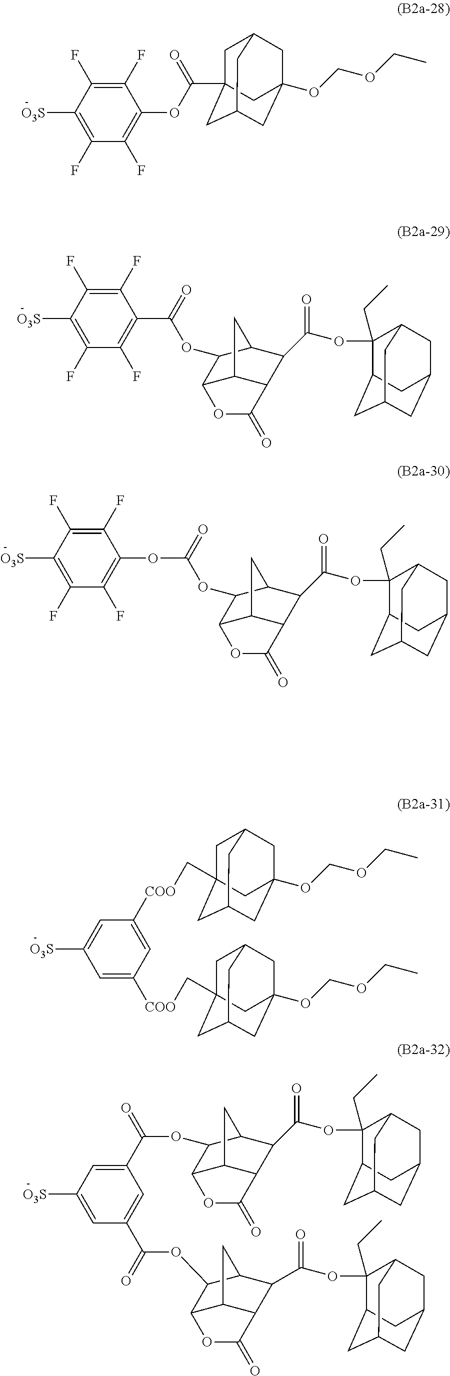

Examples of the acid generator (B2) include salts represented by the following formula.

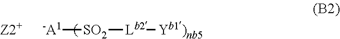

[In the formula (B2),
A¹ represents a nitrogen atom or a carbon atom.

L^{b2′} represents a single bond or a divalent hydrocarbon group having 1 to 24 carbon atoms and optionally having a substituent, and —CH₂— included in the hydrocarbon group may be substituted with —O—, —S—, —CO, —SO—, or —SO₂—.

Yb^{1′} represents a methyl group which may have a substituent or a cyclic hydrocarbon group having 3 to 24 carbon atoms which may have a substituent, and —CH₂— included in the cyclic hydrocarbon group may be replaced by —O—, —S—, —CO—, —SO—, or —SO₂—.

nb5 represents an integer of 2 or 3. When A¹ is a nitrogen atom, nb5 is 2, and when A¹ is a carbon atom, nb5 is 3. A plurality of groups in parentheses may be the same as or different from each other, and two groups in parentheses may be bonded to form a ring including A¹.

Z2⁺ represents an organic cation]

In the formula (B2), examples of those as for L^{b2′} and Y^{b1′} include the same groups as for L^{b2} and Y^{b1} of the formula (B1-A1).

L^{b2′} is preferably a single bond, *-L^{b23-}, *-L^{b23}-X²- or *-L^{b23}-X²-W²-X³-(L^{b23} represents a chain hydrocarbon group having 1 to 6 carbon atoms and optionally having a fluorine atom, X² and X³ each independently represent —O—, —CO—O—, —O—CO—, —O—CO—O— or —O—, W² represents an alicyclic hydrocarbon group having 3 to 12 carbon atoms, —CH₂— included in the alicyclic hydrocarbon group may be substituted with —O— or —CO—, and * represents a binding site to SO₂).

Y^{b1′} is preferably a methyl group having a fluorine atom or a cyclic hydrocarbon group having 3 to 20 carbon atoms and optionally having a substituent (—CH₂— included in the cyclic hydrocarbon group may be substituted with —O—, —CO—, —S—, —SO—, or —SO₂—), and is more preferably a trifluoromethyl group, an alicyclic hydrocarbon group having 3 to 18 carbon atoms and optionally having a substituent (—CH₂— included in the alicyclic hydrocarbon group may be substituted with —O—, —S—, —CO—, —SO—, or —SO₂—) or an aromatic hydrocarbon group having 6 to 18 carbon atoms and optionally having a substituent. Specifically, a trifluoromethyl group or a group represented by the formula (Y1) to the formula (Y36) exemplified in the formula (B1-A1) is preferable.

In the anion of the salt represented by the formula (B2), when two —SO₂-L^{b2′}-Y^{b1′} combine to form a ring containing A¹, examples thereof include an anion represented by the formula (B2′).

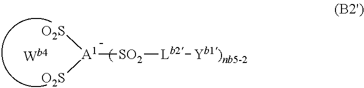

[In the formula (B2′),
A¹, Y^{b1′}, L^{b2′}, and nb5 are the same as defined in formula (B2).

W^{b4} represents a disulfonylimide ring or disulfonylmethide ring having 2 to 12 carbon atoms and optionally having a fluorine atom.]

The disulfonylimide ring or disulfonylmethide ring as for W^{b4} preferably has 3 to 12 carbon atoms, and more preferably 3 to 6 carbon atoms, and the hydrogen atom of the methylene group included in the ring is preferably substituted with a fluorine atom.

Examples of the anion of the salt represented by the formula (B2) include the following. Among them, anions represented by a formula (B3a-1), a formula (B3a-2), and a formulas (B3a-12) to a formula (B3a-14) are preferable.

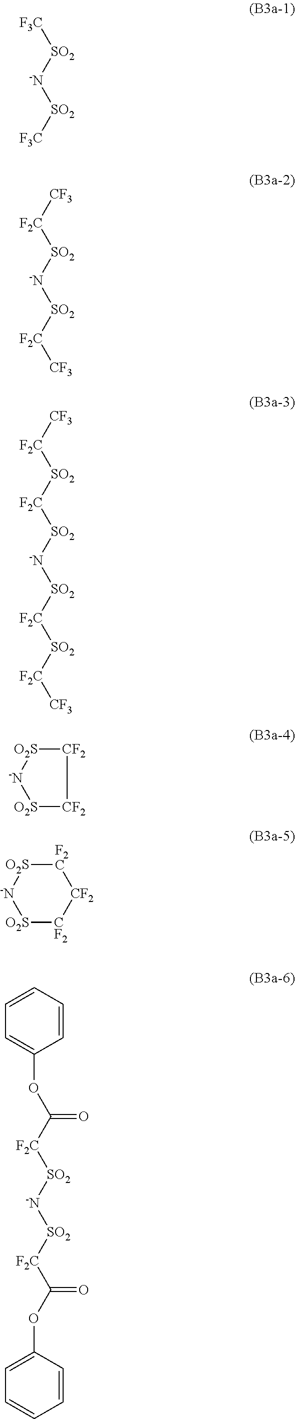

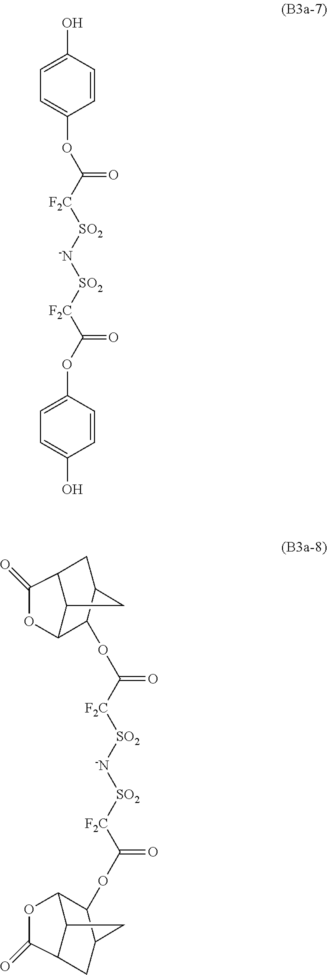

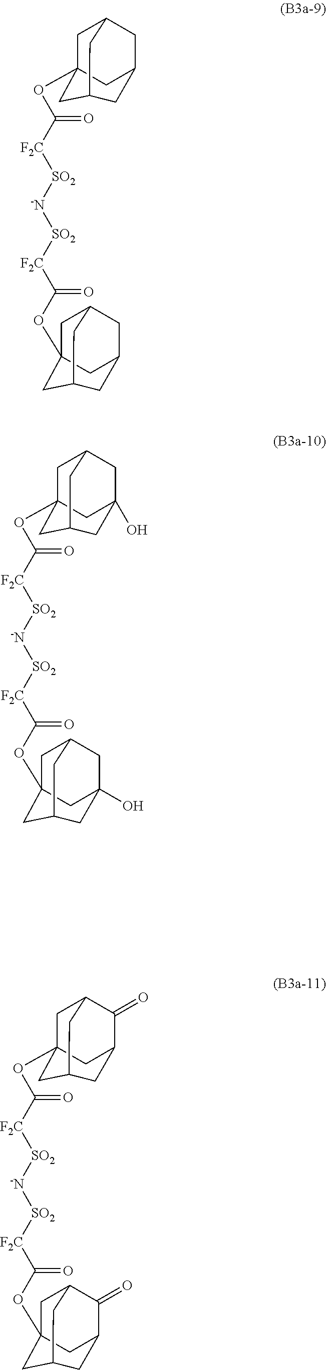

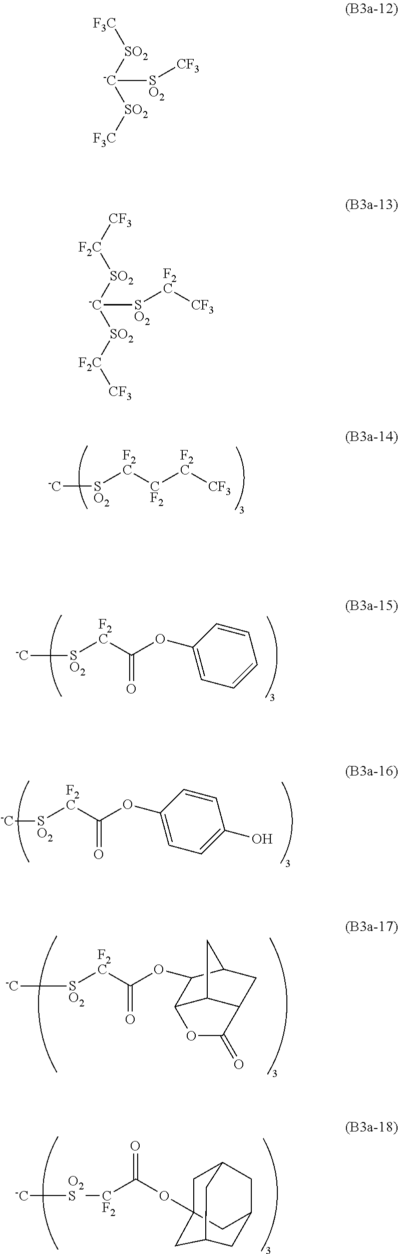

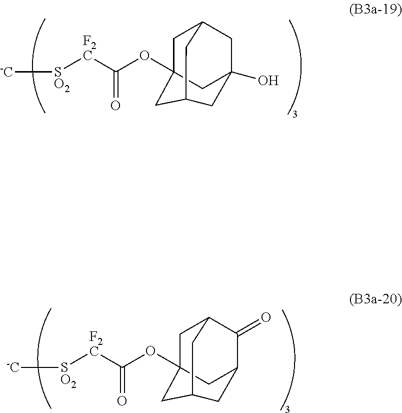

Examples of the acid generator (B34) include salts represented by the following formula.

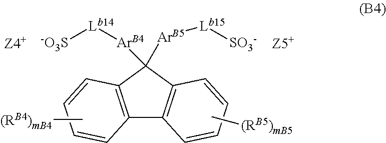

[In the formula (B4),
L^{b14} and L^{b15} each independently represent a single bond or a divalent hydrocarbon group having 1 to 72 carbon atoms and optionally having a substituent, and —CH₂— included in the hydrocarbon group may be substituted with —O—, —S—, —CO—, —SO—, or —SO₂—.

Ar^{B4} and Ar^{B5} each independently represent an aromatic hydrocarbon group having 6 to 24 carbon atoms and optionally having a substituent, and substituents of Ar^{B4} and Ar^{B5} may be bonded to each other to form a ring.

R^{B4} and R^{B5} each independently represent a halogen atom or a hydrocarbon group having 1 to 18 carbon atoms and optionally having a substituent, and —CH₂— included in the hydrocarbon group may be substituted with —O—, —CO—, —S—, —SO—, or —SO₂—.

mB4 represents an integer of 0 to 4, and when mB4 is 2 or more, a plurality of R^{B4} may be the same as or different from each other.

mB5 represents an integer of 0 to 4, and when mB5 is 2 or more, a plurality of R^{B5} may be the same as or different from each other.

Z4⁺ and Z5⁺ each independently represent an organic cation.]

In the formula (B4), examples of L^{b14} and L^{b15} include the same group as L^{b1} of the formula (B1) or L^{b3} of the formula (B1-A1), and examples of L^{b24} and L^{b25} include the same group as L^{b2} of the formula (B1) or the formula (B1-A1).

Examples of the aromatic hydrocarbon group of Ar^{B4} and Ar^{B5} include a phenylene group, a naphthylene group, an anthrylene group, a biphenylene group, and a phenanthrylene group. The binding site can be any position. The number of carbon atoms of the aromatic hydrocarbon group is preferably 6 to 18, more preferably 6 to 14, and still more preferably 6 to 10.

Examples of the substituent that may be possessed by the aromatic hydrocarbon group of Ar^{B4} and Ar^{B5} include a halogen atom, a hydroxy group, a cyano group, a carboxy group, an alkyl group having 1 to 12 carbon atoms, a fluorinated alkyl group having 1 to 6 carbon atoms, an alkoxy group having 1 to 12 carbon atoms, and a group obtained by combining two or more of these groups. Examples of these substituents include the same substituents as those exemplified in R^{B4} and R^{B5} described later.

The ring formed by the substituents of Ar^{B4} and Ar^{B5} together may be any ring composed of Ar^{B4} and Ar^{B5} together to which the substituents are bonded, and may include a heteroatom or the like as in the case exemplified by the substituents. Specific examples thereof include a ring in which —CH₂— included in a fluorene ring, a benzofluorene ring, a dibenzofluorene ring, a dihydroanthracene ring, a xanthene ring, a thioxanthene ring, an anthrone ring, or a dihydroanthracene ring is substituted with —SO—, —SO₂—, or the like.

Among them, Ar^{B4} and Ar^{B5} are preferably an aromatic hydrocarbon group having 6 to 10 carbon atoms and optionally having a substituent, more preferably an aromatic hydrocarbon group having 6 to 10 carbon atoms and optionally having a halogen atom, a hydroxy group, an alkyl group having 1 to 12 carbon atoms, or a fluorinated alkyl group having 1 to 6 carbon atoms, still more preferably an aromatic hydrocarbon group having 6 to 10 carbon atoms and optionally having a fluorine atom, an iodine atom, an alkyl group having 1 to 6 carbon atoms, or a fluorinated alkyl group having 1 to 3 carbon atoms, and still more preferably a phenylene group or a naphthylene group.

When Ar^{B4} and Ar^{B5} are phenylene groups optionally having a substituent, the bonding position of the fluorene ring may be any of an ortho position, a meta position, and a para position with respect to the bonding position of L^{b14} and L^{b15}, but is preferably a meta position or a para position, and more preferably a para position.

Examples of the halogen atom as for R^{B4} and R^{B5} include a fluorine atom, a chlorine atom, a bromine atom, and an iodine atom.

Examples of the hydrocarbon group having 1 to 18 carbon atoms as for R^{B4} and R^{B5} include a chain hydrocarbon group such as an alkyl group and an alkanediyl group, an alicyclic hydrocarbon group, an aromatic hydrocarbon group, and a group formed by combining these groups, and examples of these groups include the same groups as those exemplified in the present specification as long as the upper limit of the carbon number is acceptable.

The number of carbon atoms of the chain hydrocarbon group is preferably 1 to 12, more preferably 1 to 9, still more preferably 1 to 6, yet more preferably 1 to 4, and further preferably 1 to 3.

The number of carbon atoms of the alicyclic hydrocarbon group is preferably 3 to 18, more preferably 3 to 16, and still more preferably 3 to 12.

The number of carbon atoms of the aromatic hydrocarbon group is preferably 6 to 18, more preferably 6 to 14, and still more preferably 6 to 10.

In combination, two or more of alicyclic hydrocarbon groups, aromatic hydrocarbon groups, and chain hydrocarbon groups may be respectively combined. In addition, any group may be bonded to the benzene ring and the like.

When —CH₂— included in the hydrocarbon group represented by R^{B4} and R^{B5} is substituted with —O—, —CO—, —S—, —SO—, or —SO₂—, the number of carbon atoms before substitution is defined as the total number of carbon atoms of the hydrocarbon group. The number may be either 1, or 2 or more.

Examples of the hydrocarbon group in which —CH₂— is substituted with —O—, —S—, —SO—, —SO₂—, or —CO— include, as described above, a hydroxy group, a thiol group, a carboxy group, an alkoxy group,
an alkoxycarbonyl group, an alkylcarbonyl group, an alkylcarbonyloxy group, an alkylthio group, an alkylsulfonyl group, an oxy group, a carbonyl group, a thio group, a sulfonyl group, an alkanediyloxy group, an alkanediyloxycarbonyl group,
an alkanediylcarbonyl group, an alkanediylcarbonyloxy group, an alkanediylthio group, an alkanediylsulfonyl group, a cycloalkoxy group, a cycloalkylalkoxy group, an alkoxycarbonyloxy group,
an aromatic hydrocarbon group-carbonyloxy group, an aromatic hydrocarbon group-carbonyl group, an aromatic hydrocarbon group-oxy group, and the group obtained by combining two or more of these groups. Examples of these substituted groups include those that are the same as described in the present specification as long as the upper limit of the number of carbon atoms permits.

Examples of the group in which —CH₂— included in the alicyclic hydrocarbon group and the group obtained by combining the alicyclic hydrocarbon group and the aromatic hydrocarbon is substituted with —O—, —S—, —SO—, —SO₂—, or —CO— include the same groups as those exemplified in the present specification as long as the upper limit of the number of carbon atoms is acceptable.

Examples of the substituent that may be possessed by the hydrocarbon group as for R^{B4} and R^{B5} include a halogen atom, a cyano group, and a nitro group.

Examples of the halogen atom include the same groups as described above.

The hydrocarbon group may have one substituent or a plurality of substituents.

When R^{B4} and R^{B5} are a hydrocarbon group having a halogen atom, examples of the hydrocarbon group having a halogen atom as for R^{B4} and R^{B5} include a chain hydrocarbon group having a halogen atom such as a haloalkyl group, an alicyclic hydrocarbon group having a halogen atom, and an aromatic hydrocarbon group having a halogen atom.

The haloalkyl group represents an alkyl group having a halogen atom, and examples thereof include haloalkyl groups having 1 to 12 carbon atoms such as a fluorinated alkyl group having 1 to 12 carbon atoms, a chloride alkyl group having 1 to 12 carbon atoms, a bromide alkyl group having 1 to 12 carbon atoms, and an iodide alkyl group having 1 to 12 carbon atoms. Examples of the haloalkyl group include a perfluoroalkyl group having 1 to 12 carbon atoms (trifluoromethyl group, pentafluoroethyl group, heptafluoropropyl group, nonafluorobutyl group, etc.), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, an iodomethyl group and the like. The number of carbon atoms of the haloalkyl group is preferably 1 to 9, more preferably 1 to 6, still more preferably 1 to 4, and yet more preferably 1 to 3.

—CH₂— included in the haloalkyl group may be substituted with —O—, —CO—, or the like, and examples of such a group include a haloalkoxy group, a haloalkoxycarbonyl group, a haloalkylcarbonyl group, and a haloalkylcarbonyloxy group. Specific examples thereof include a haloalkoxy group having 1 to 11 carbon atoms, a haloalkoxycarbonyl group having 2 to 11 carbon atoms, a haloalkylcarbonyl group having 2 to 12 carbon atoms, and a haloalkylcarbonyloxy group having 2 to 11 carbon atoms, and examples thereof include groups in which one or more hydrogen atoms of the groups exemplified above are substituted with halogen atoms.

mB4 is preferably 0, 1, 2, or 3, more preferably 0, 1, or 2, and still more preferably 0 or 1.

mB5 is preferably 0, 1, 2, or 3, more preferably 0, 1, or 2, and still more preferably 0 or 1.

R^{B4} and R^{B5} are each independently preferably a halogen atom, a haloalkyl group having 1 to 6 carbon atoms, or an alkyl group having 1 to 6 carbon atoms (—CH₂— included in the haloalkyl group and the alkyl group may be substituted with —O— or —CO—), more preferably a fluorine atom, an iodine atom, a haloalkyl group having 1 to 6 carbon atoms, or an alkyl group having 1 to 6 carbon atoms (—CH₂— included in the haloalkyl group and the alkyl group may be substituted with —O— or —CO—), still more preferably a fluorine atom, an iodine atom, a haloalkyl group having 1 to 4 carbon atoms, or an alkyl group having 1 to 4 carbon atoms (—CH₂— included in the alkyl group may be substituted with —O— or —CO—), yet more preferably a fluorine atom, an iodine atom, a hydroxy group, an alkyl group having 1 to 4 carbon atoms, a haloalkyl group having 1 to 4 carbon atoms, or an alkoxy group having 1 to 3 carbon atoms, further preferably a fluorine atom, an iodine atom, a hydroxy group, an alkyl group having 1 to 4 carbon atoms, a perfluoroalkyl group having 1 to 4 carbon atoms, or an alkoxy group having 1 to 3 carbon atoms, and further more preferably a fluorine atom, an iodine atom, a trifluoromethyl group, a methyl group, a methoxy group, an ethoxy group, or a hydroxy group.

Examples of the anion of the salt represented by formula (B4) include the following. Among them, anions represented by a formula (B4a-1) to a formula (B4a-4) and a formula (B4a-11) to a formula (B4a-14) are preferable.

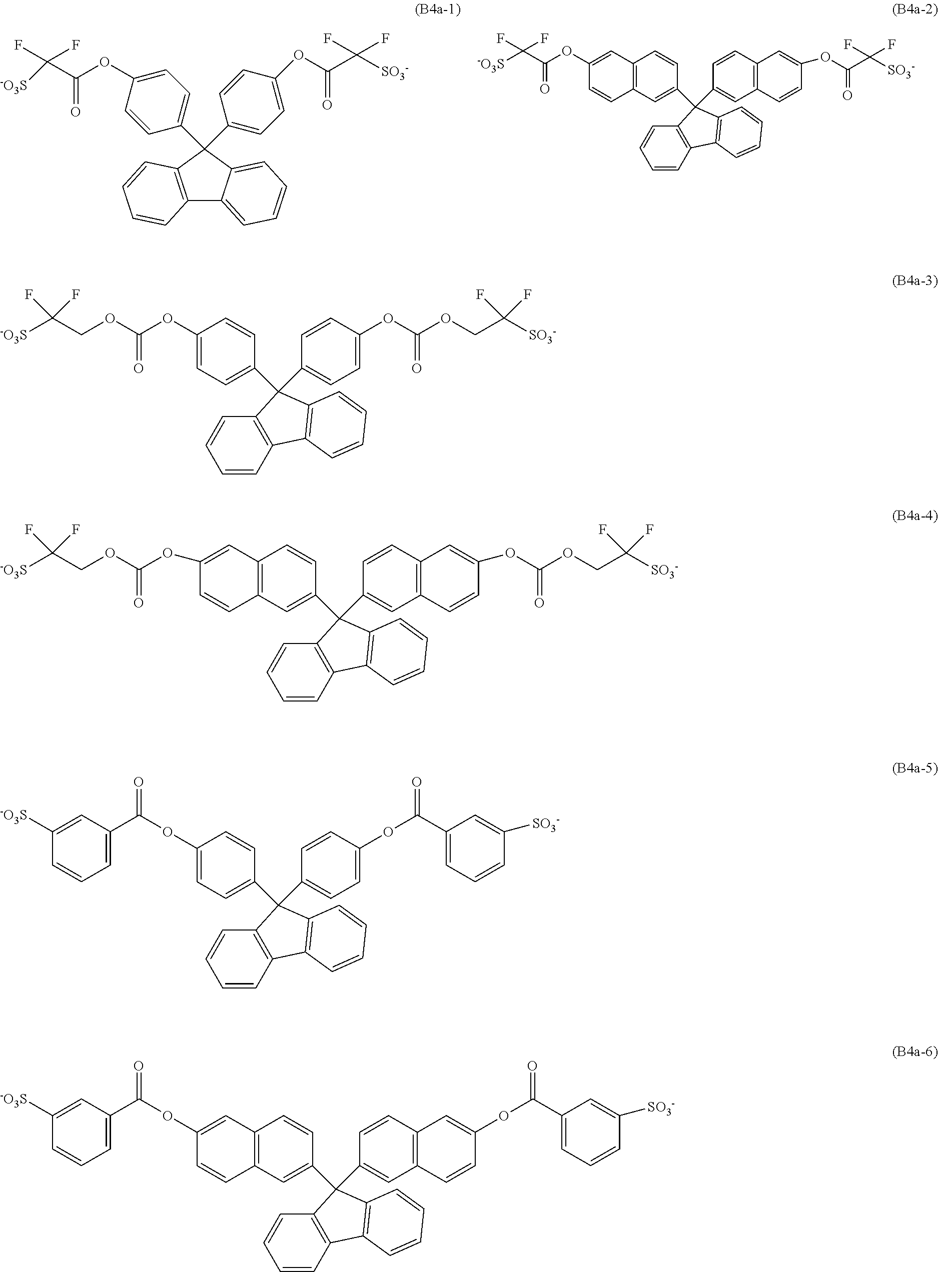

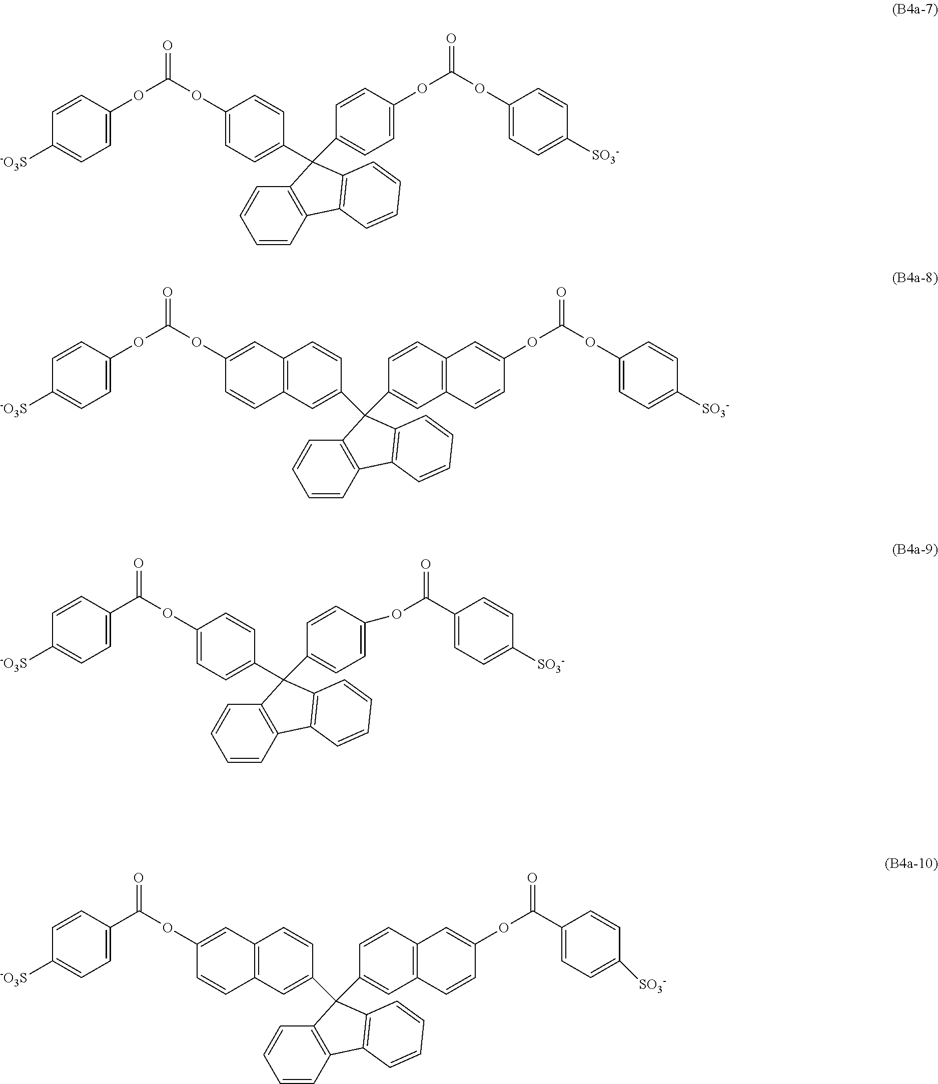

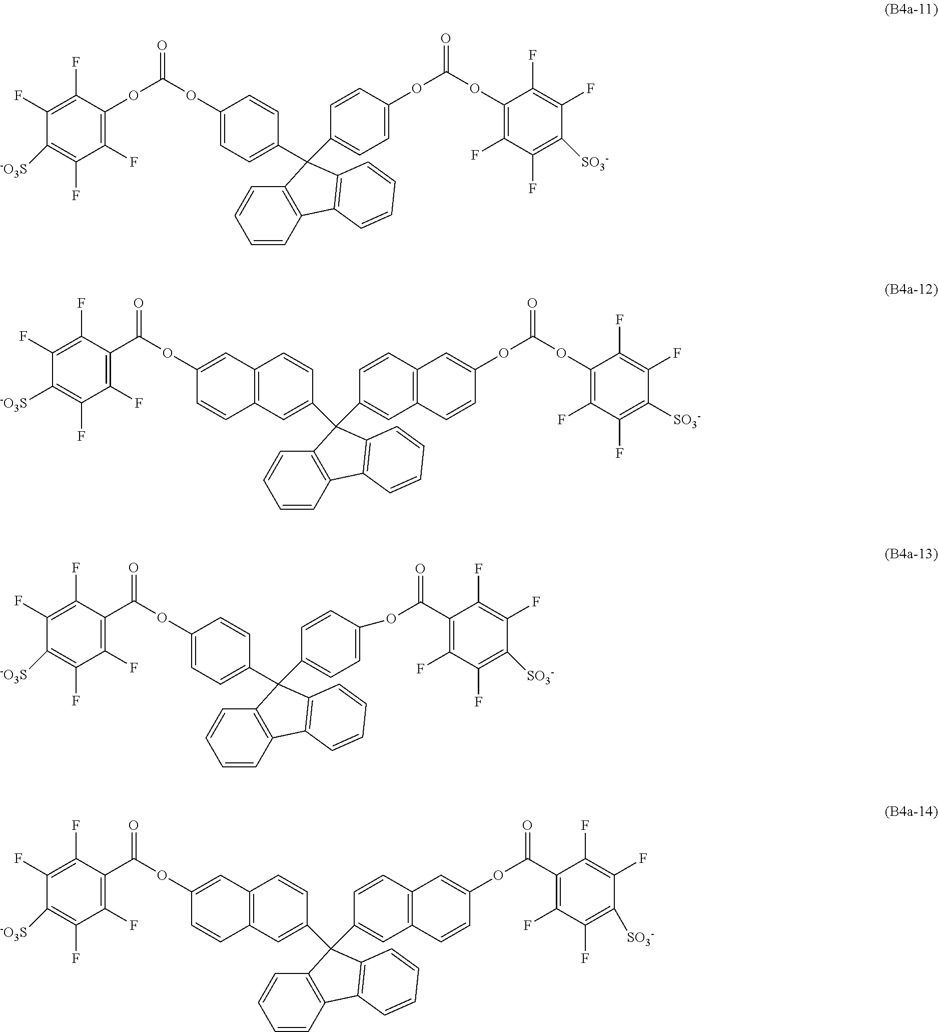

In addition, in another form of the compound (B), a salt obtained by substituting the anion in the salt represented by the formula (B 1) with a carboxylic acid anion is suitably used as the compound (B).

Examples of the carboxylic acid anion include the following.

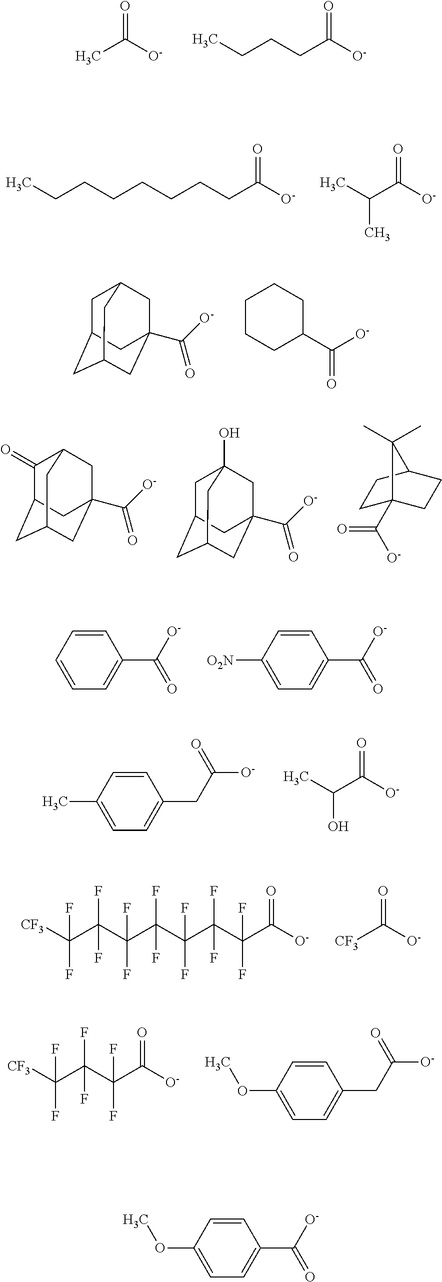

Examples of the organic cation of Z1⁺, Z2⁺, Z4⁺, and Z5⁺ include the same organic cations as those of the organic cation of Z⁺ in the formula (I).

The compound (B) is a combination of the anion described above and the organic cation described above, and these can be optionally combined. The compound (B) is preferably a combination of an anion represented by any one of a formula (B1a-1) to a formula (B1a-4), a formula (B1a-7) to a formula (B1a-11), a formula (B1a-14) to a formula (B1a-30), a formula (B1a-35) to a formula (B1a-70), a formula (B2a-1) to a formula (B2a-11), a formula (B2a-16) to a formula (B2a-20), a formula (B3a-1) to a formula (B3a-3), a formula (B3a-11) to a formula (B3a-14), a formula (B4a-1) to a formula (B4a-4), or a formula (B4a-11) to a formula (B4a-14) with a cation (b2-1), a cation (b2-2), a cation (b2-3), a cation (b2-4), or a cation (b2-5).

Preferable examples of the compound (B) include compounds represented by a formula (B1-1) to a formula (B1-106), a formula (B2-1) to a formula (B2-20), a formula (B3-1) to a formula (B3-28), and a formula (B4-1) to a formula (B4-16). Among these, those including an arylsulfonium cation are preferable, and those represented by the formula (B1-1) to a formula (B1-3), a formula (B1-5) to a formula (B1-7), a formula (B1-11) to a formula (B1-14), a formula (B1-20) to a formula (B1-26), a formula (B1-29), a formula (B1-31) to a formula (B1-105), a formula (B2-1) to a formula (B2-20), a formula (B3-1) to a formula (B3-28), and a formula (B4-1) to a formula (B4-16) are particularly preferable.

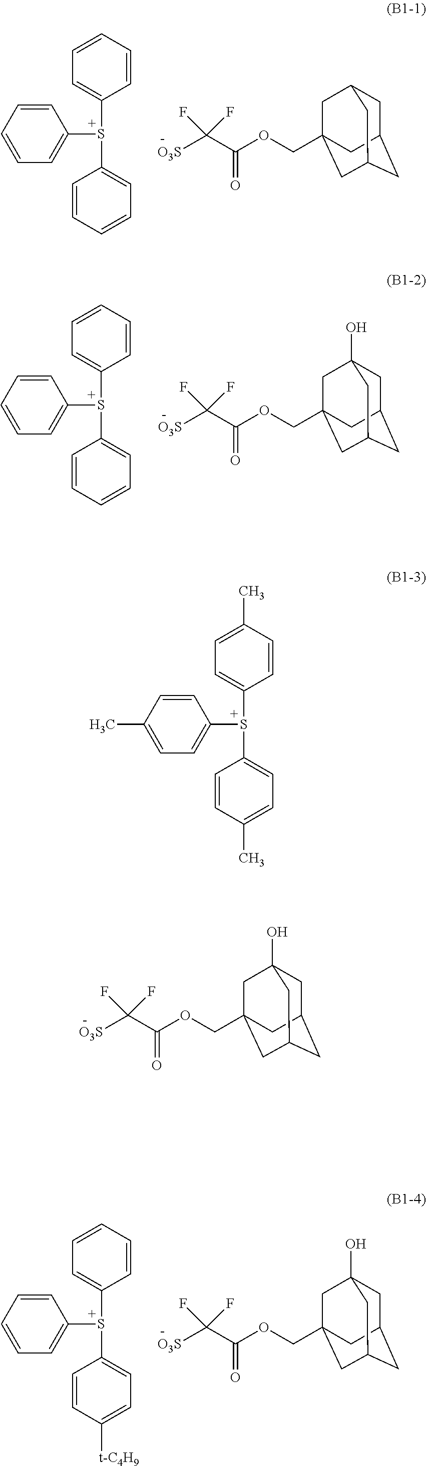

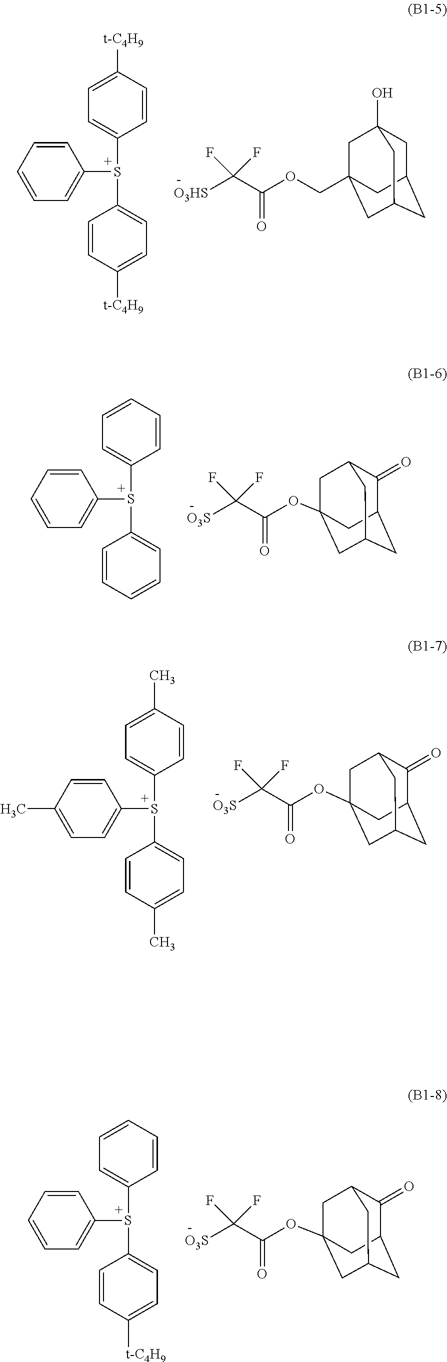

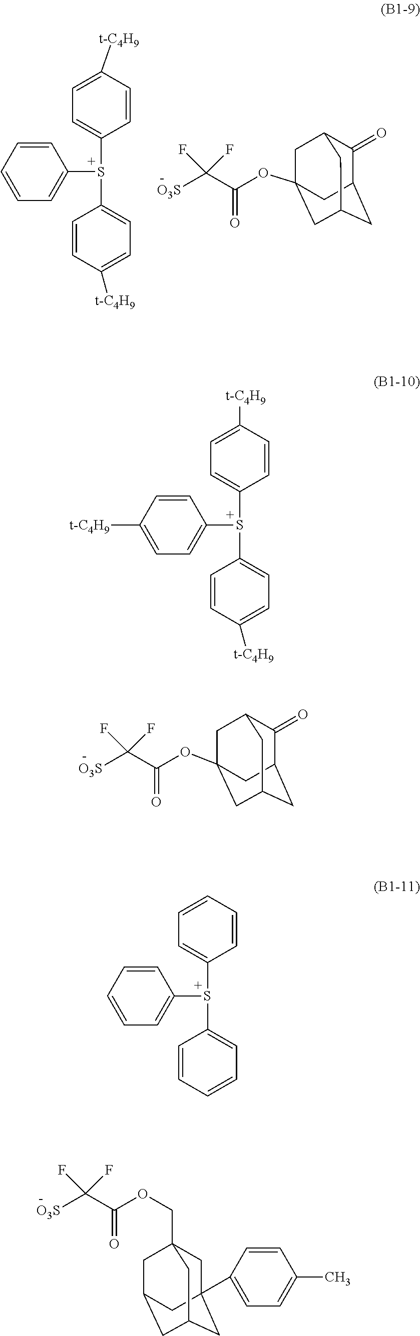

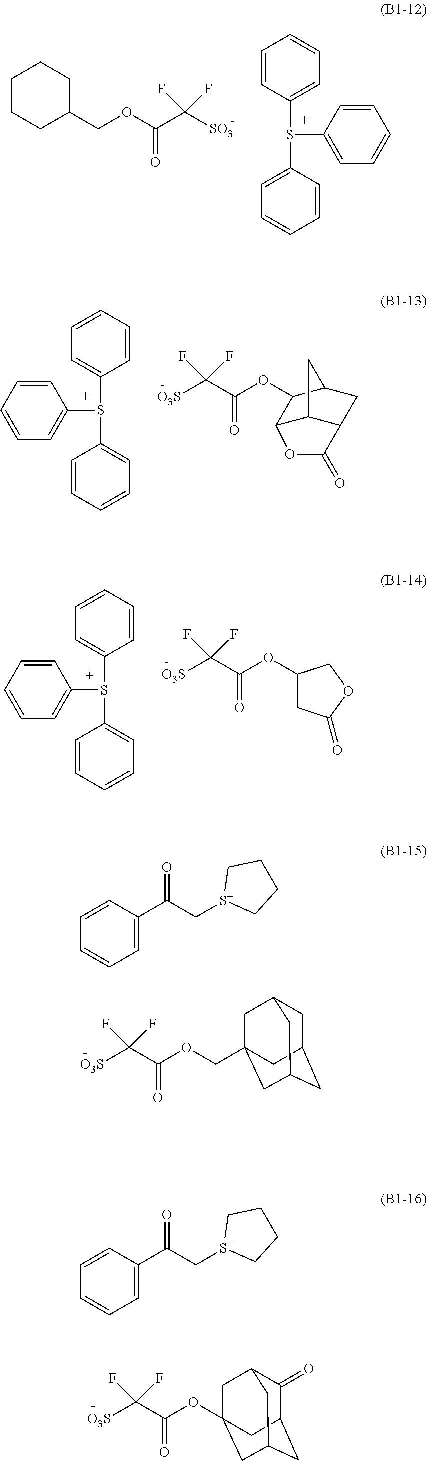

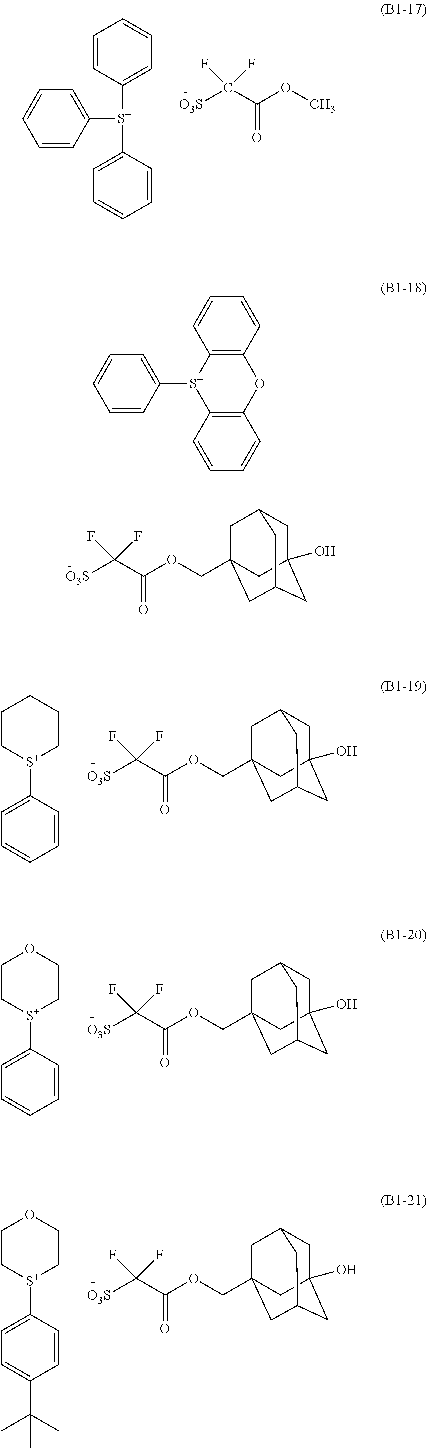

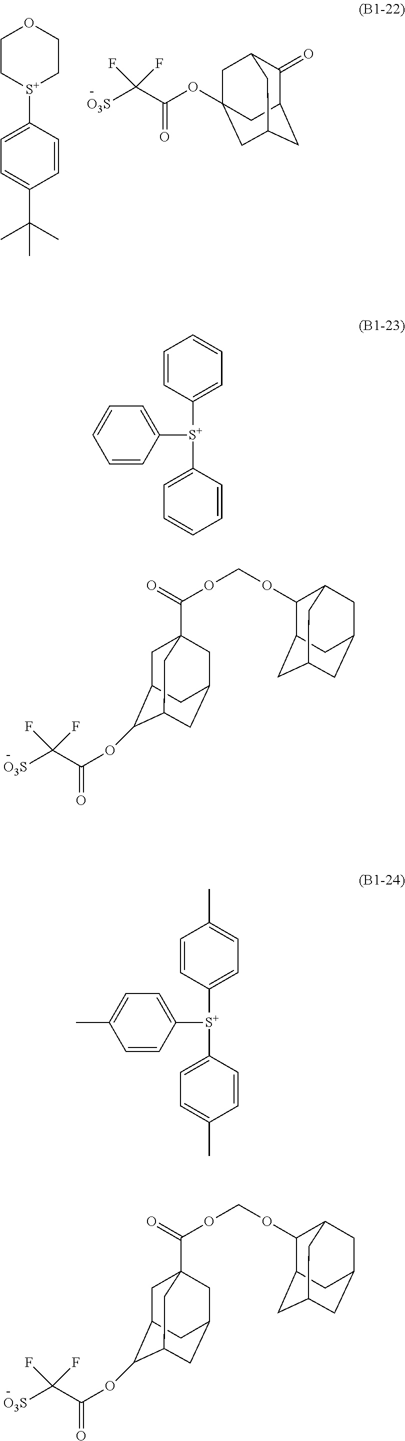

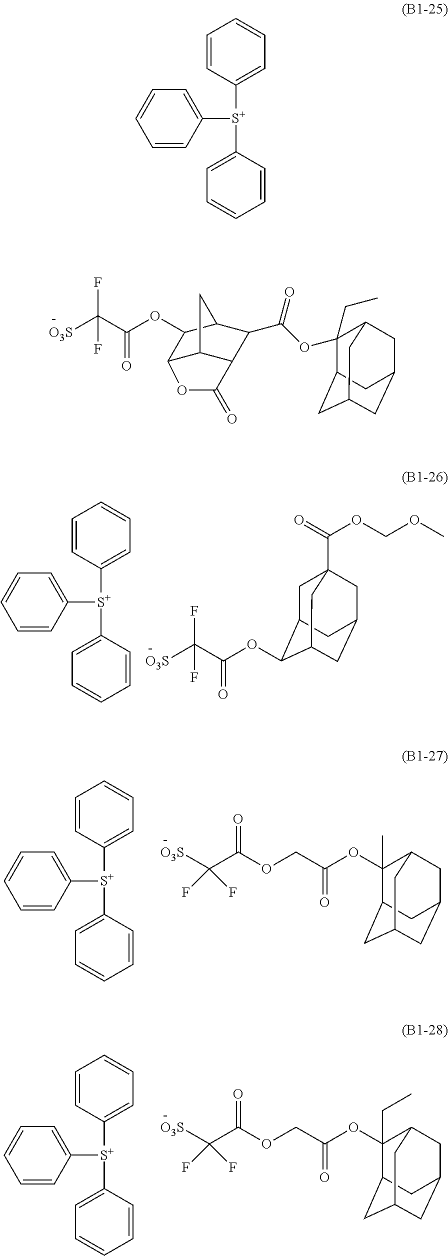

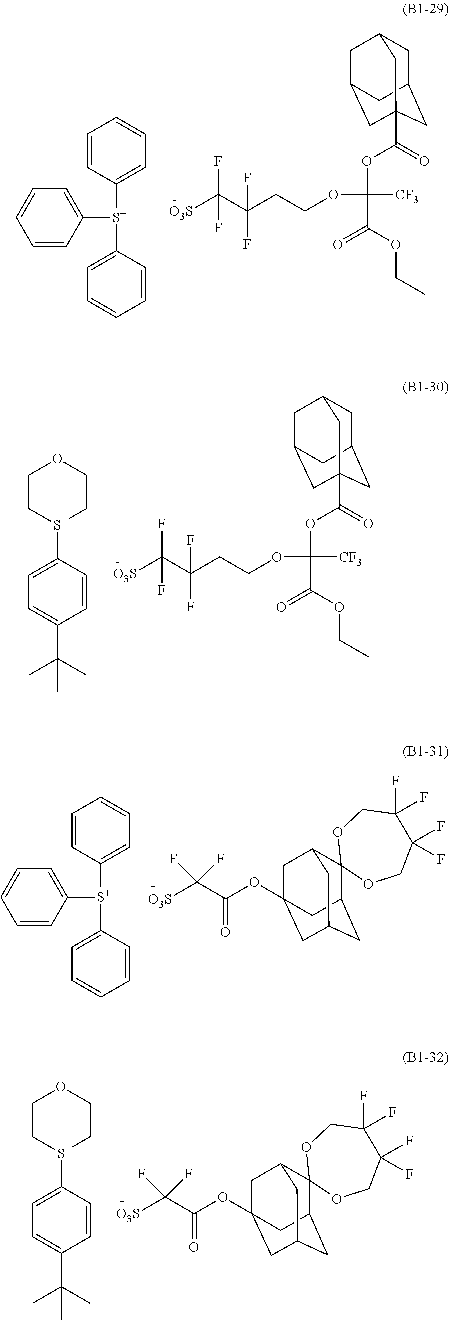

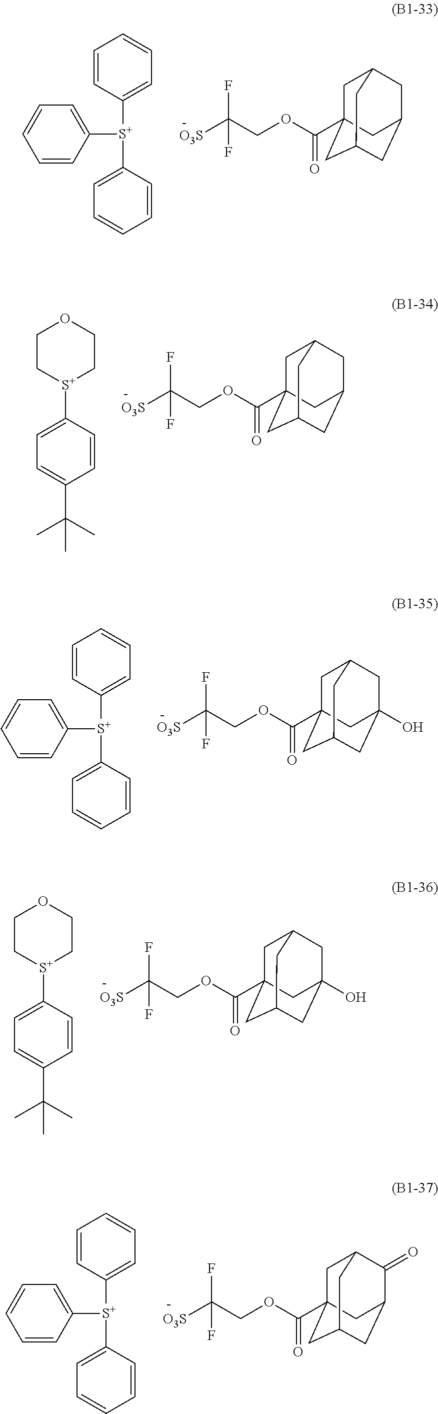

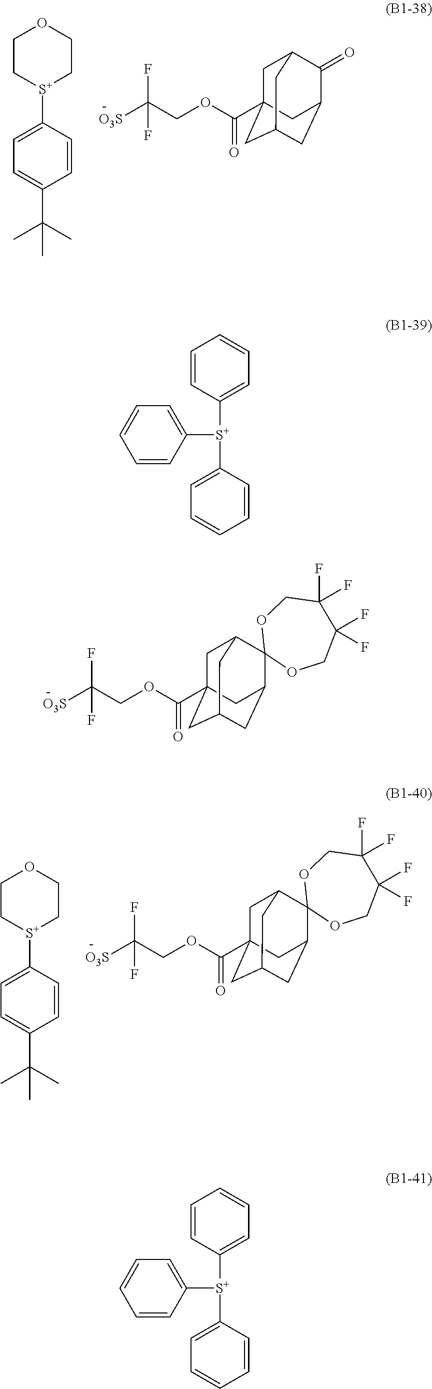

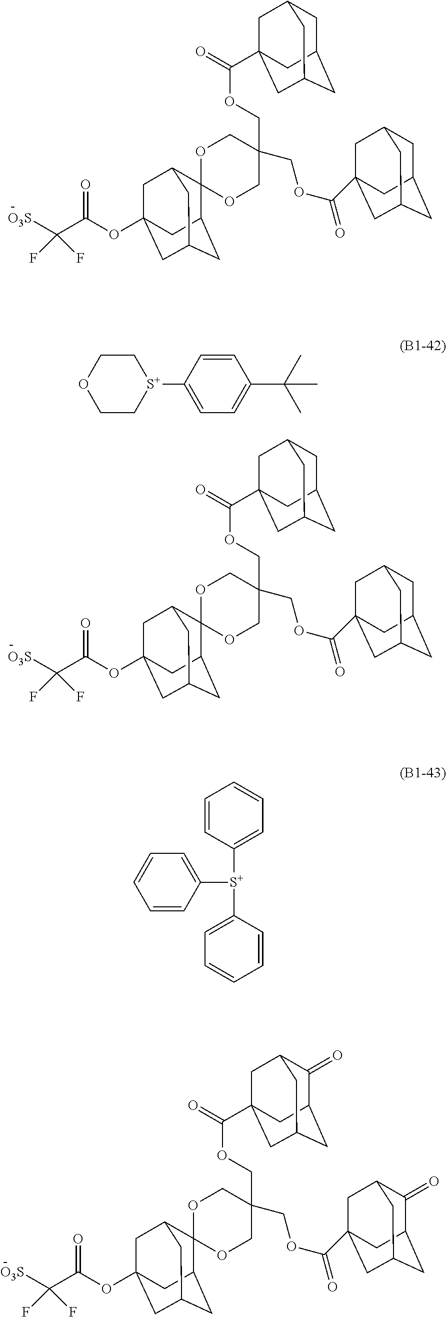

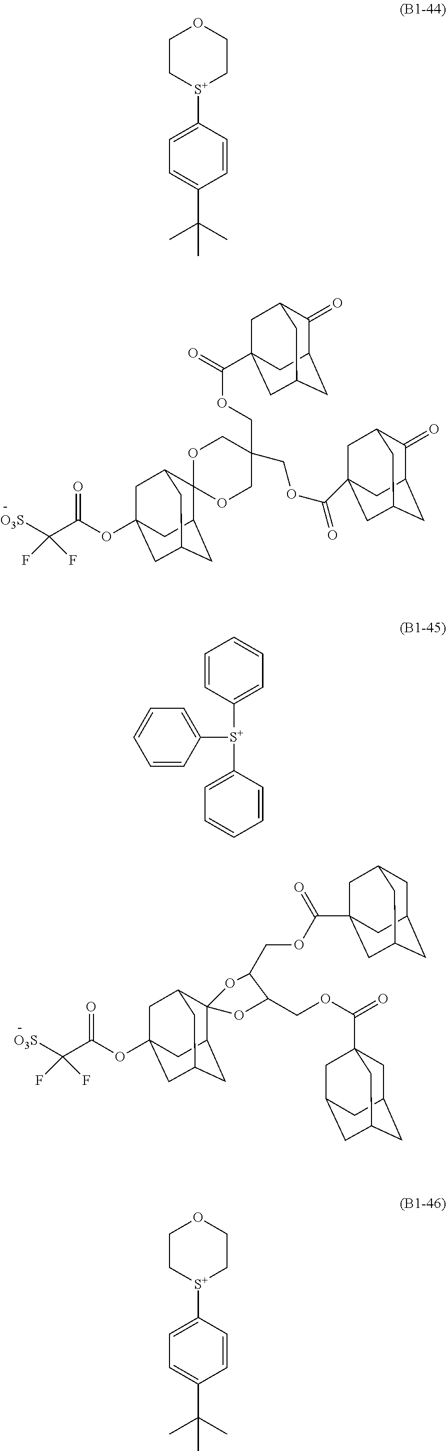

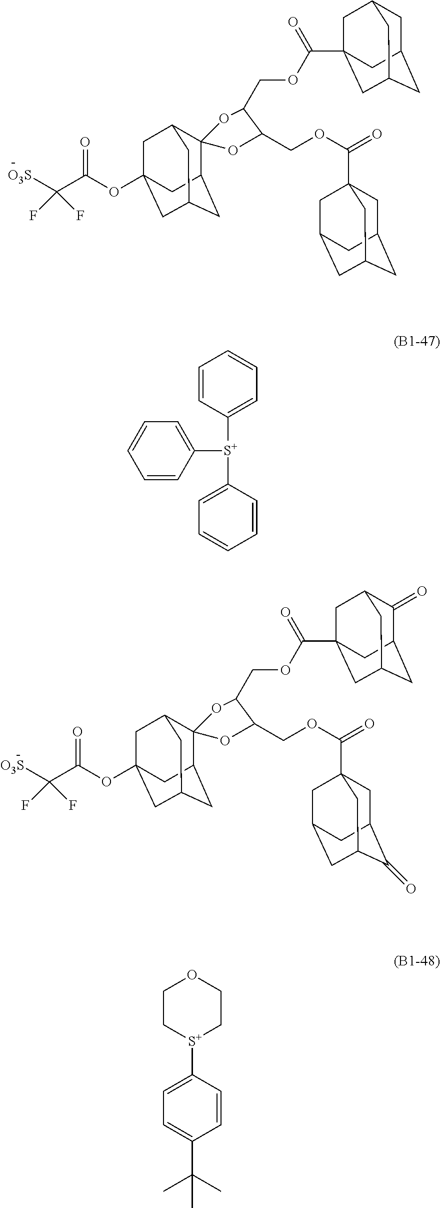

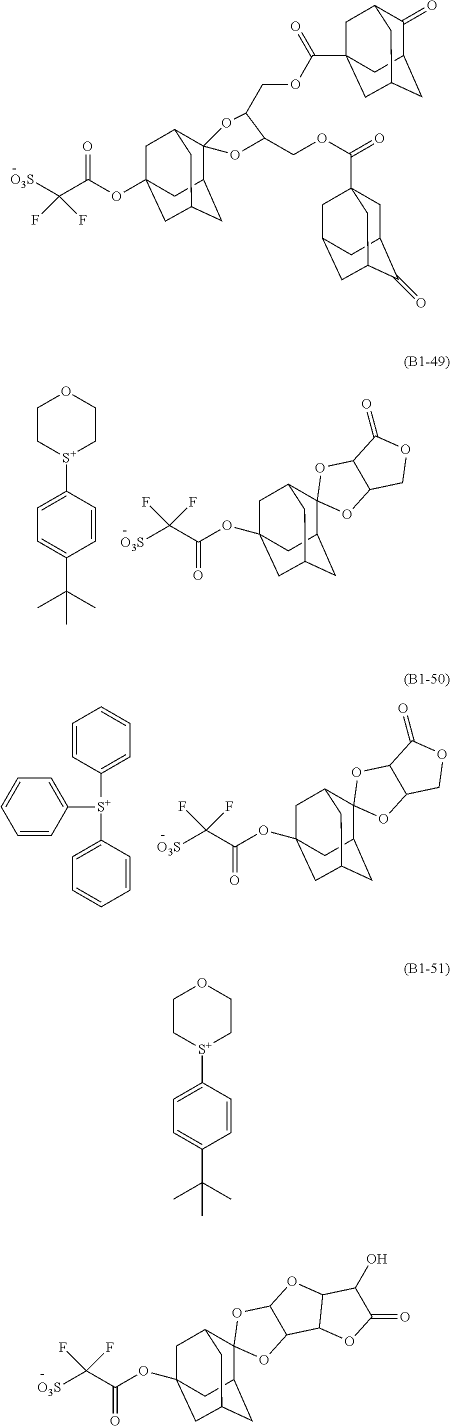

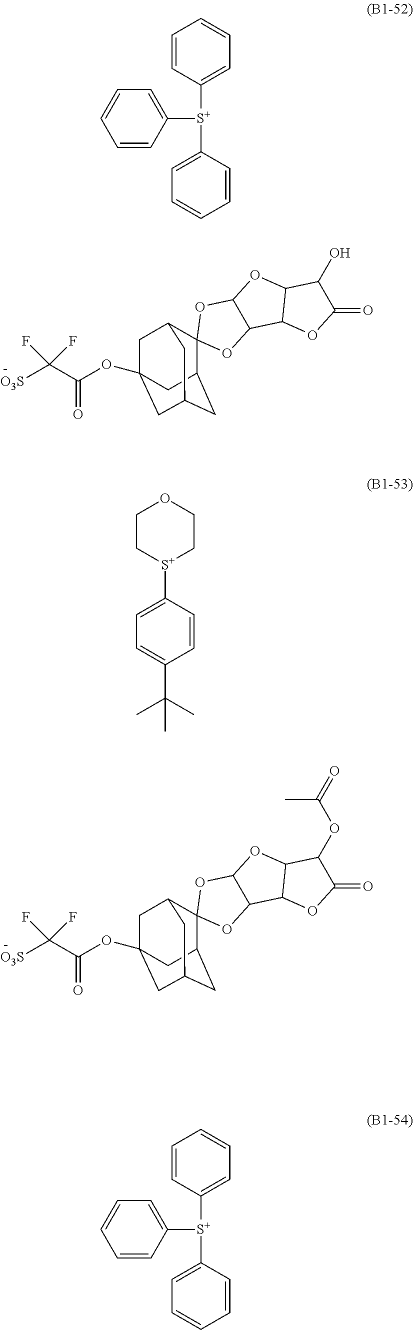

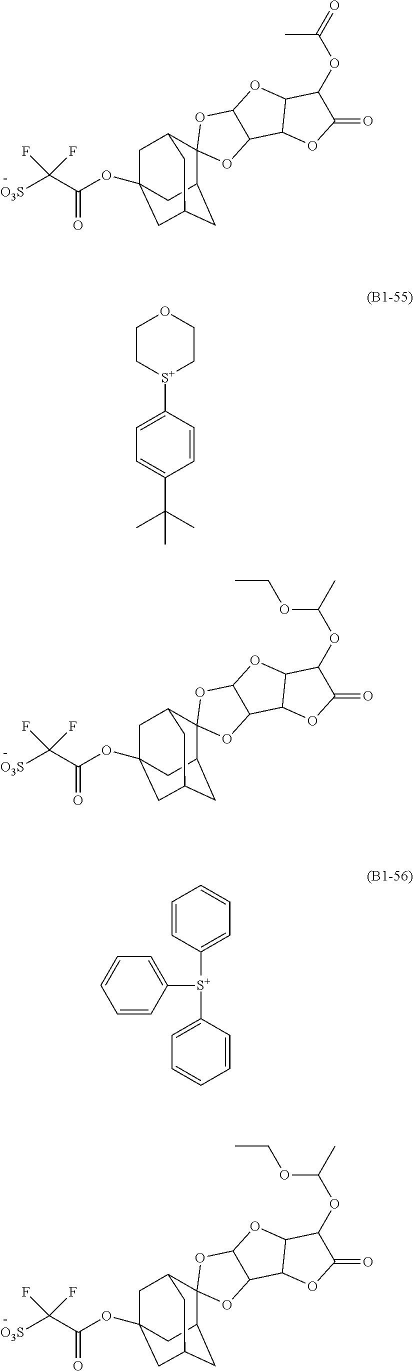

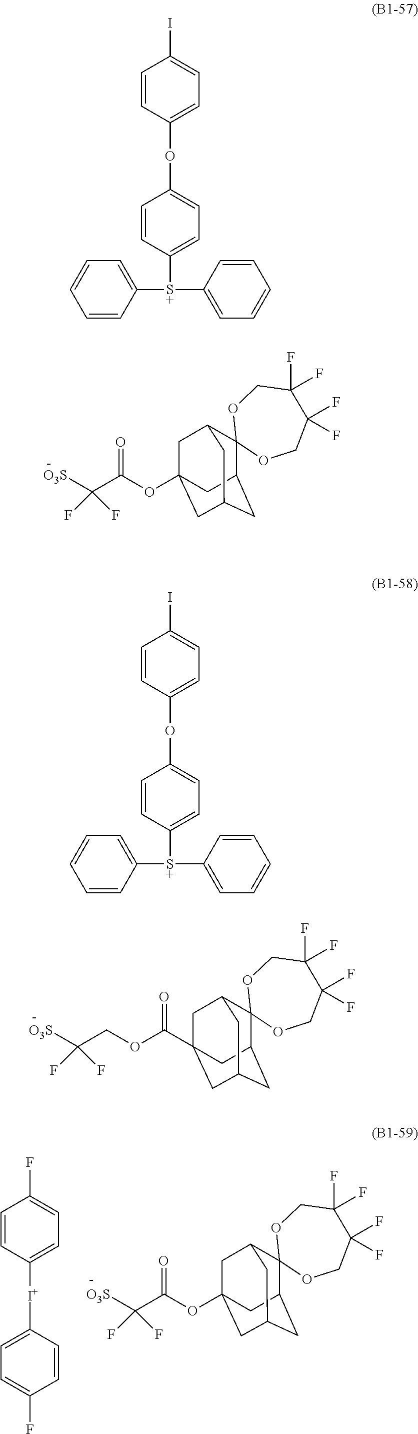

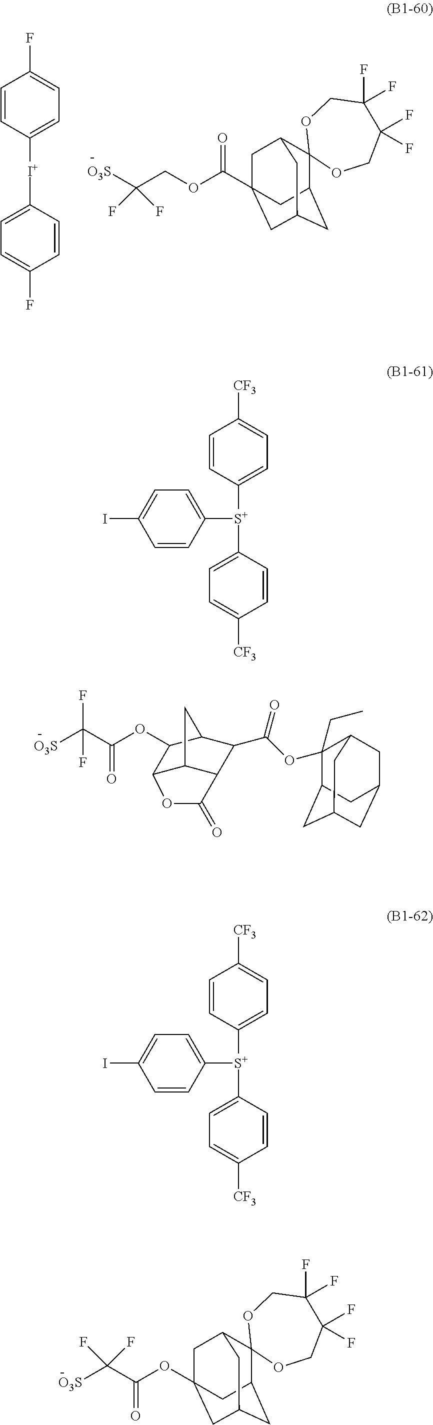

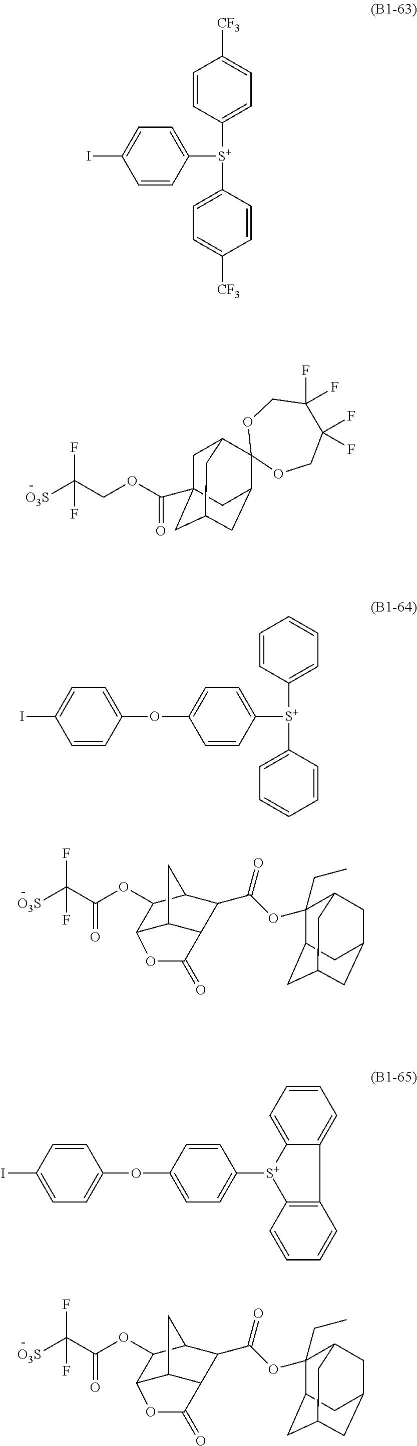

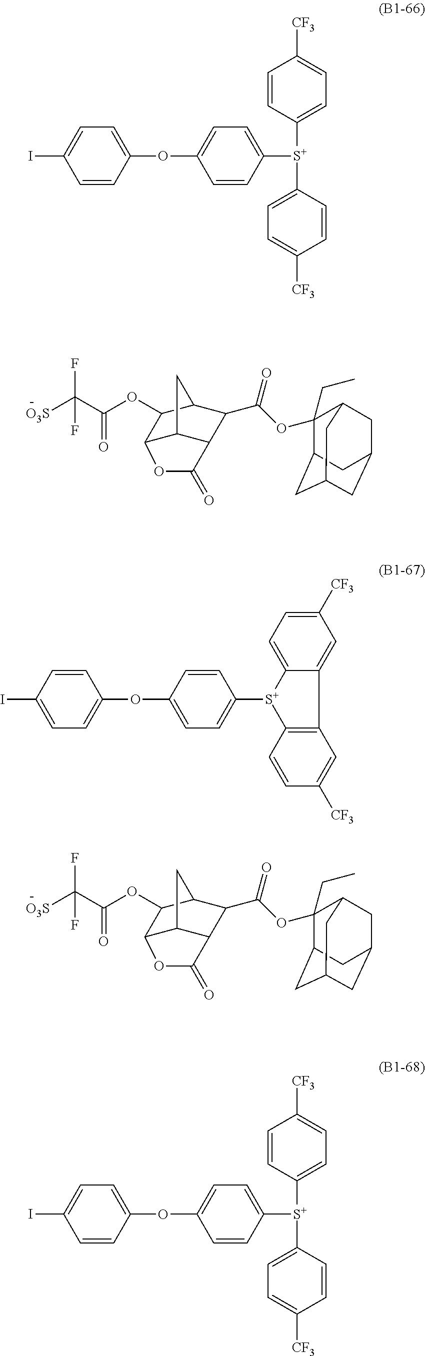

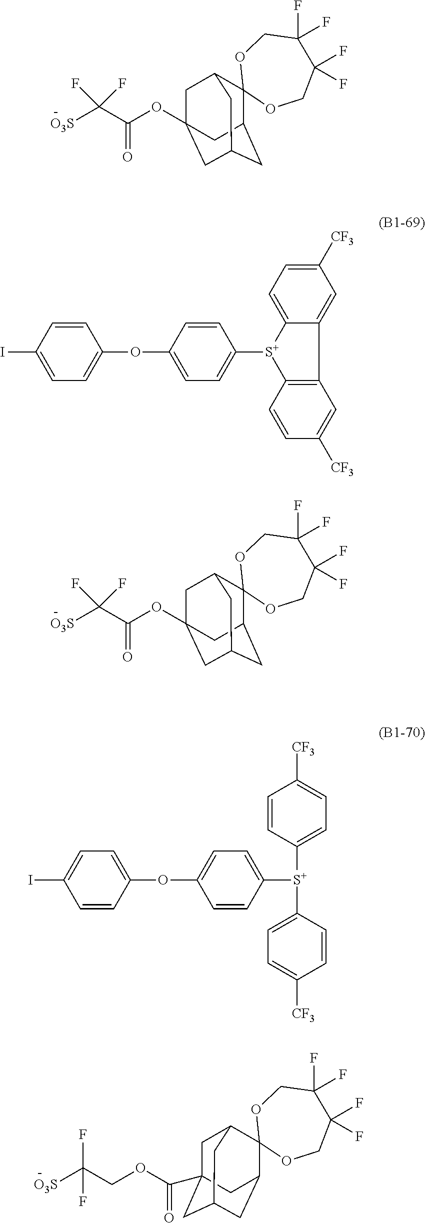

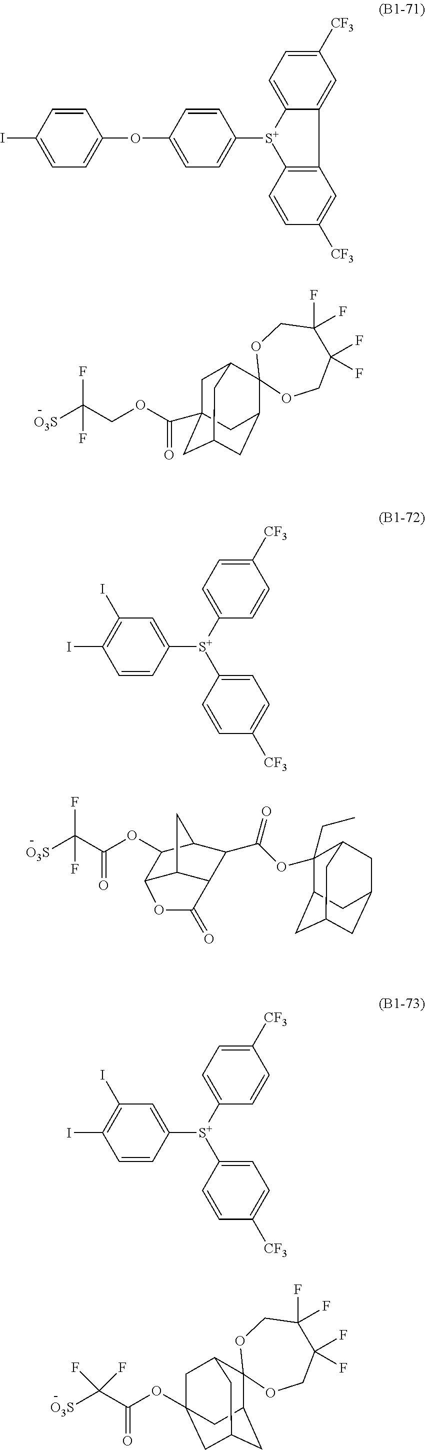

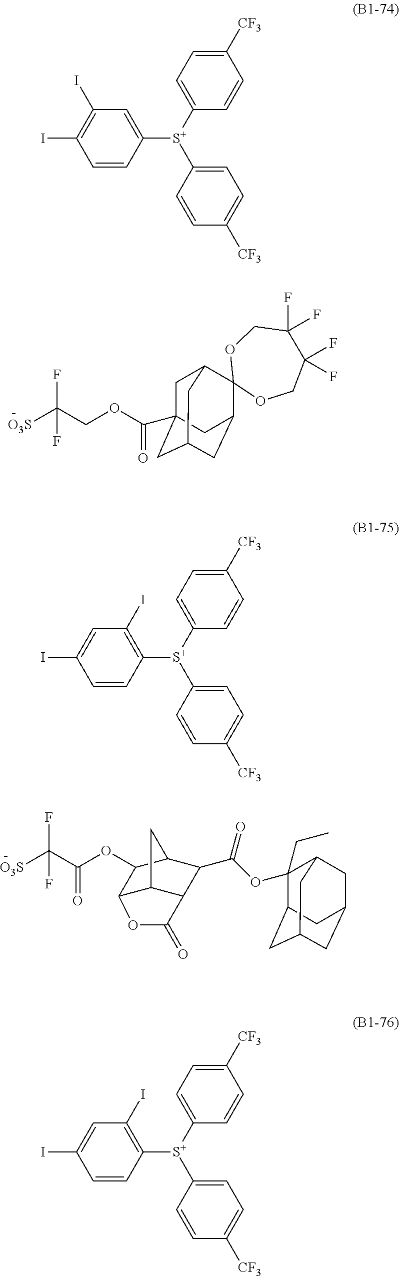

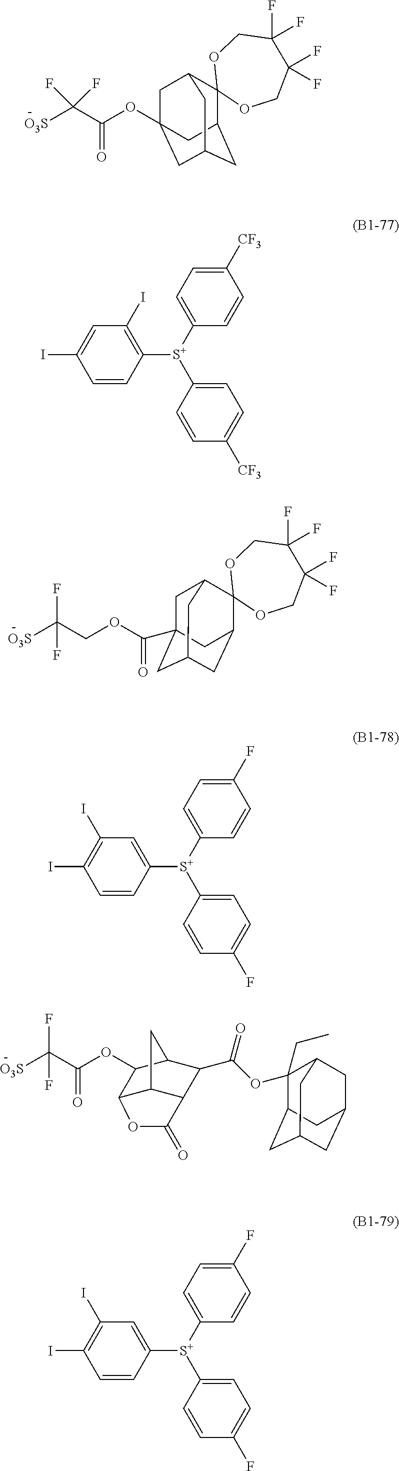

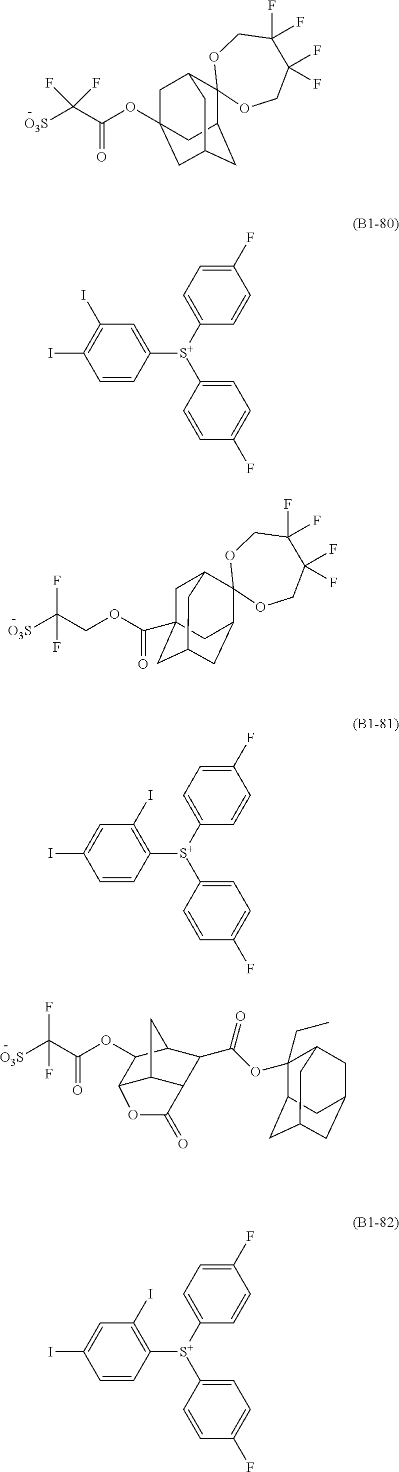

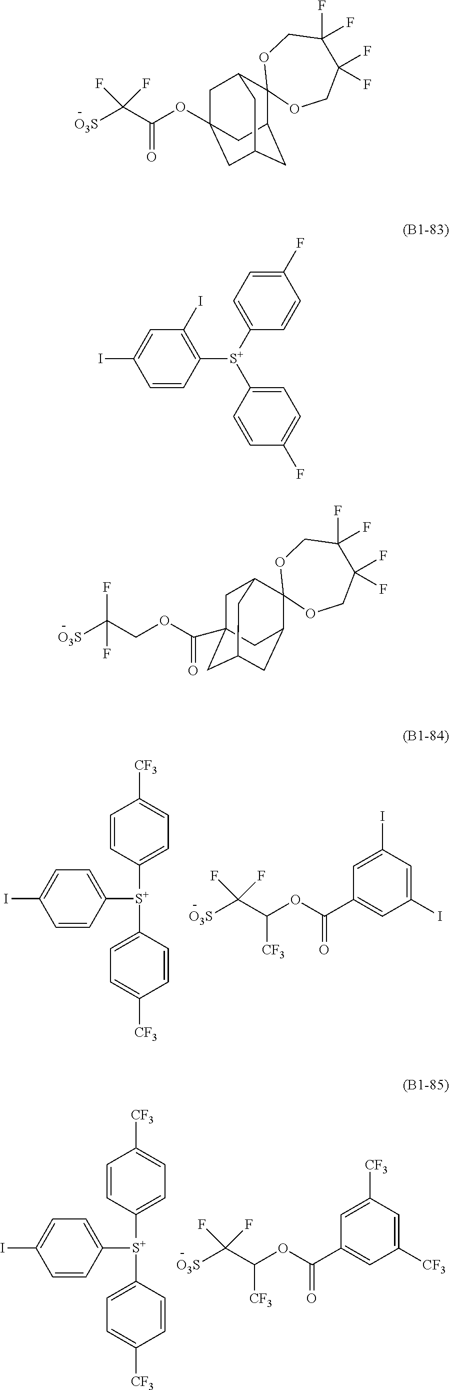

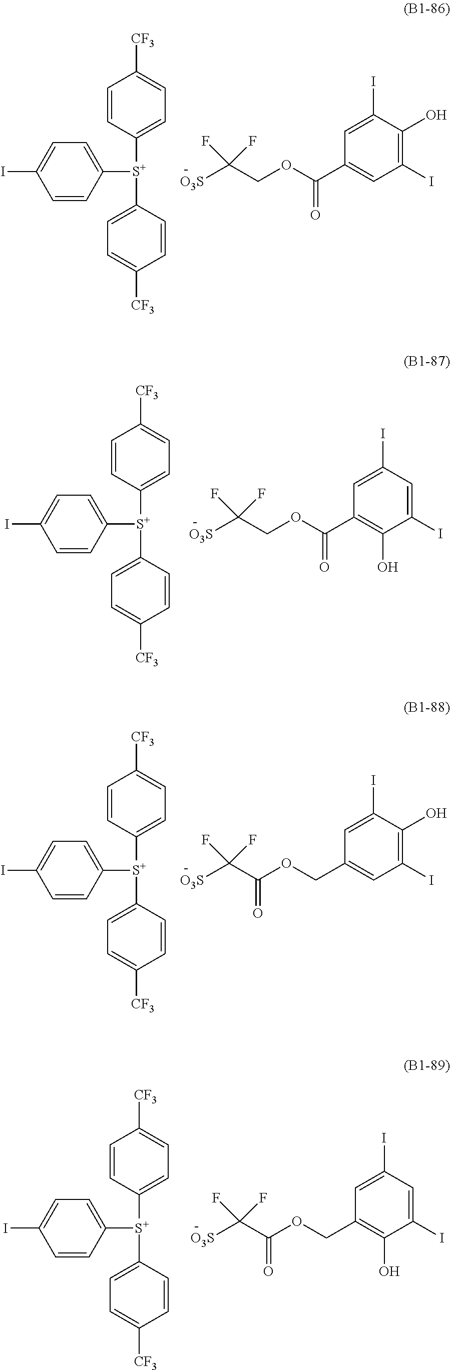

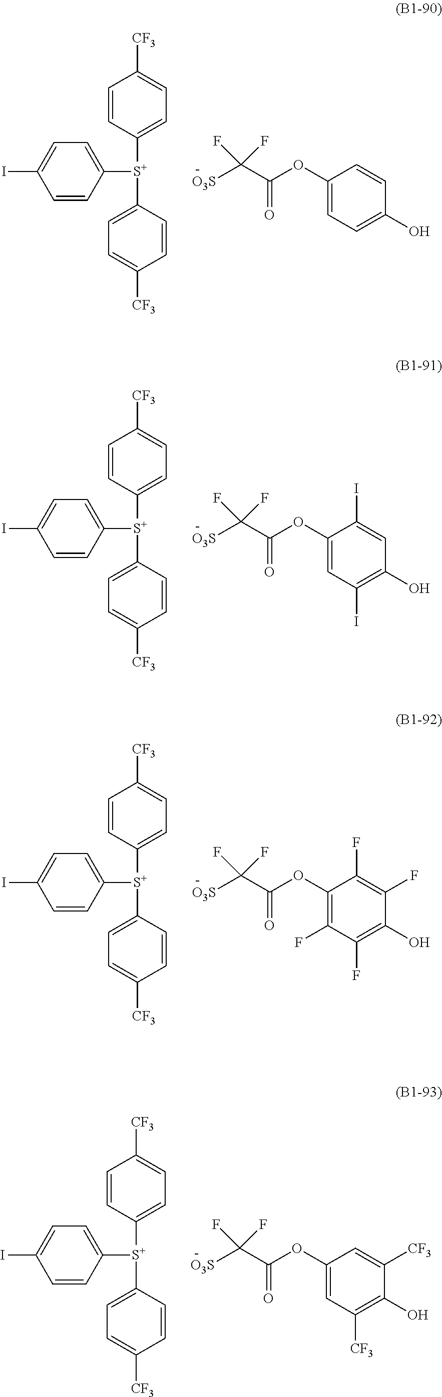

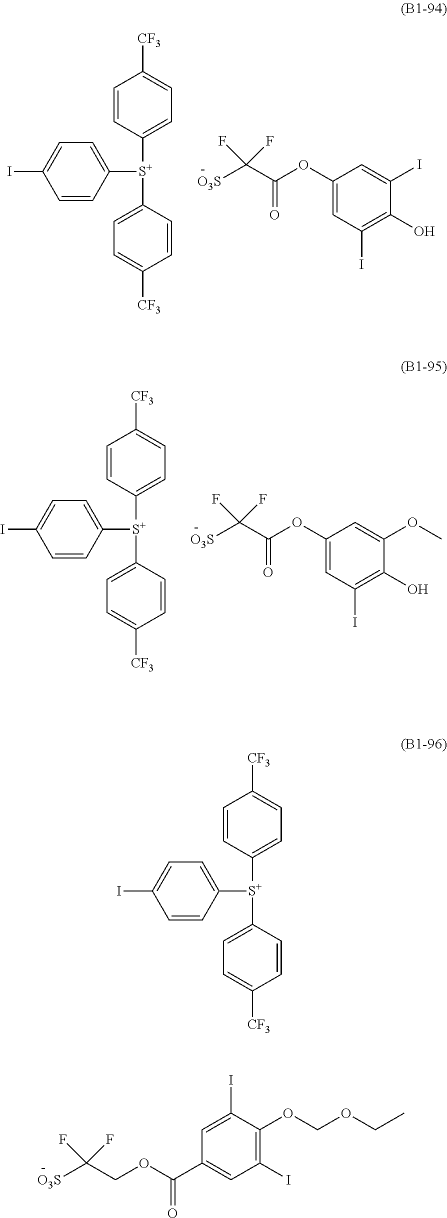

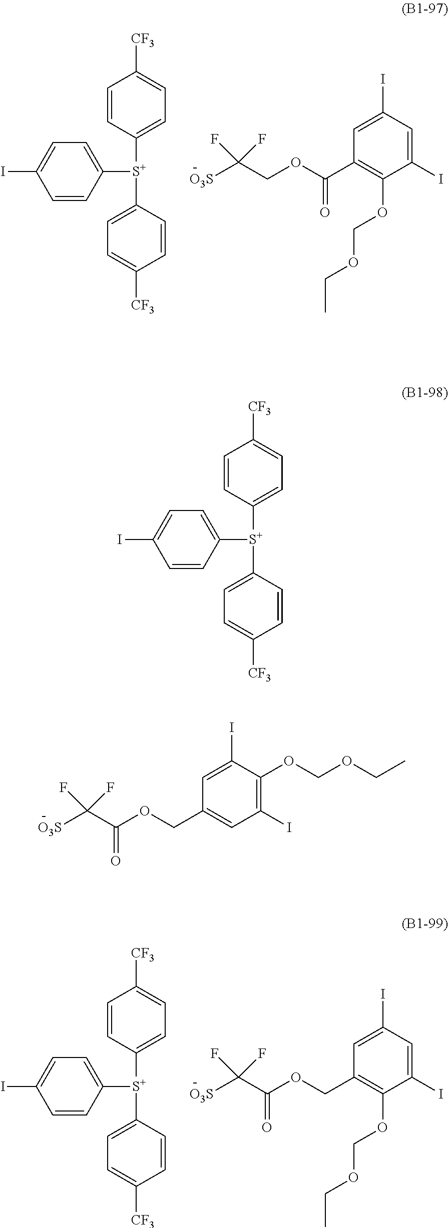

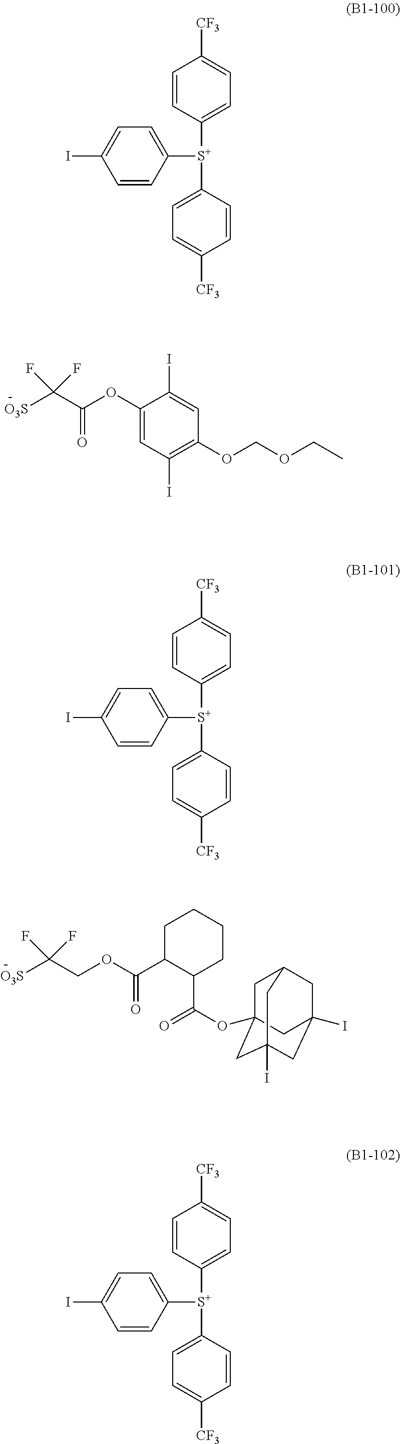

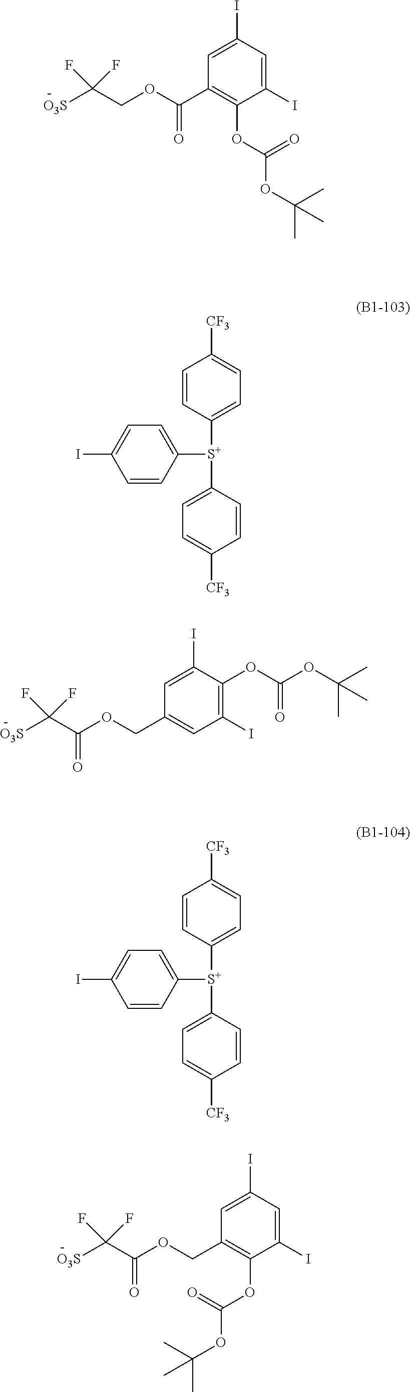

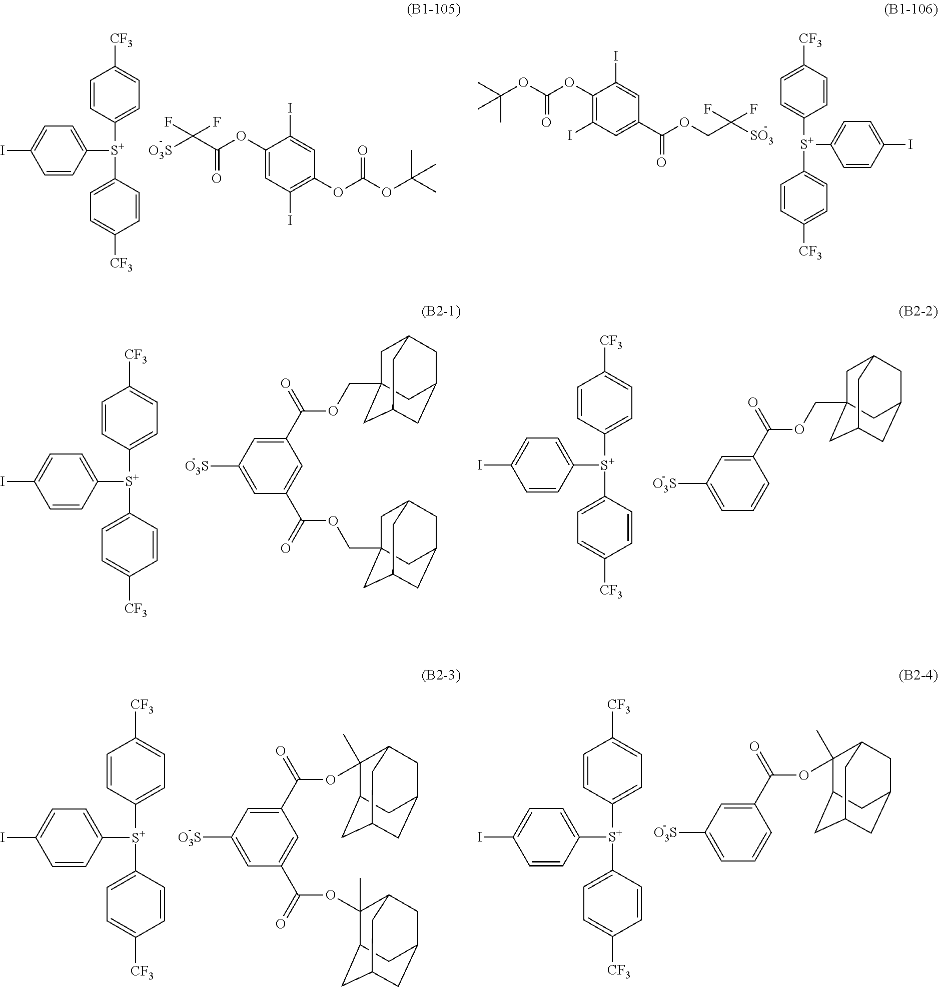

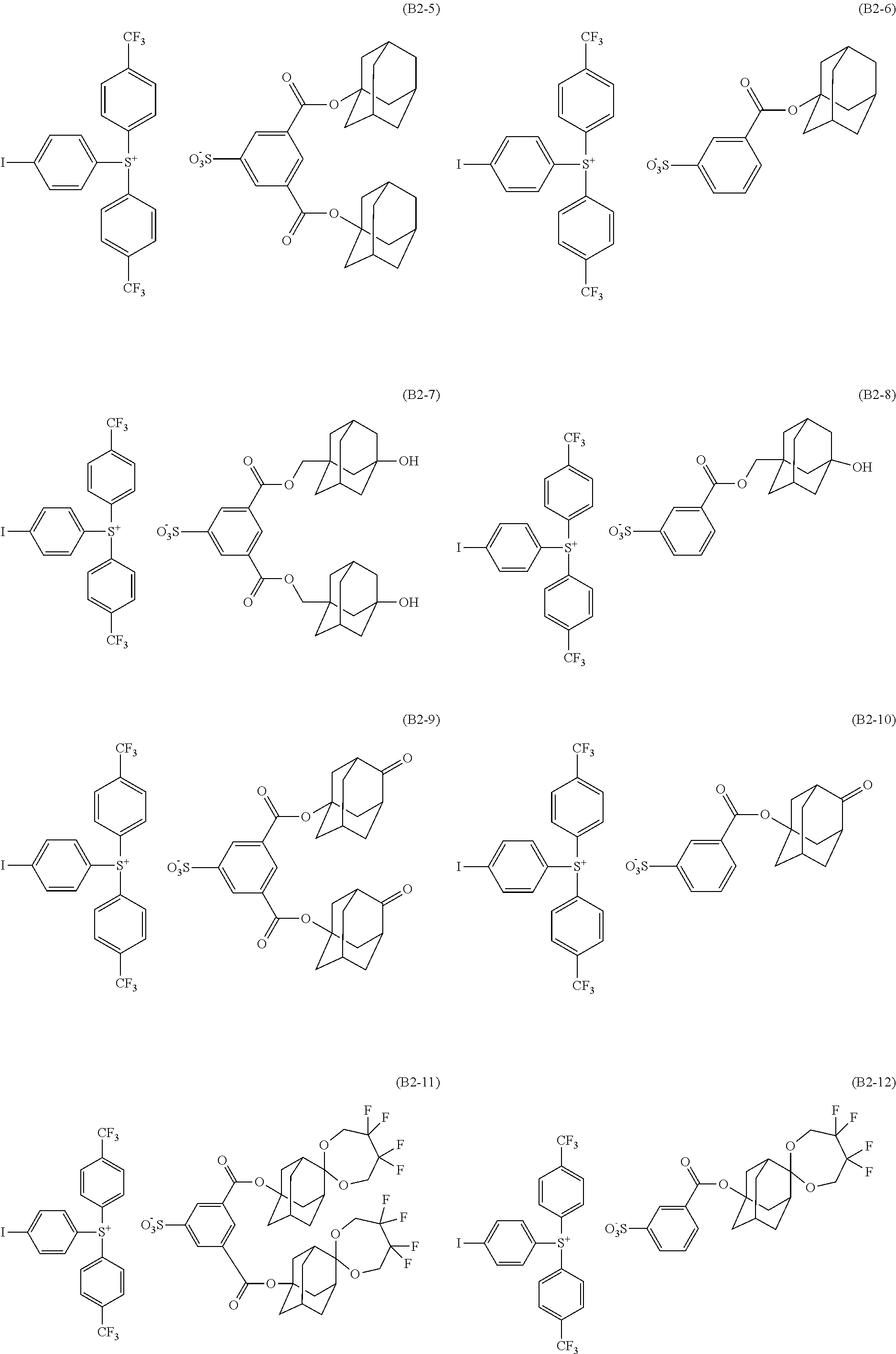

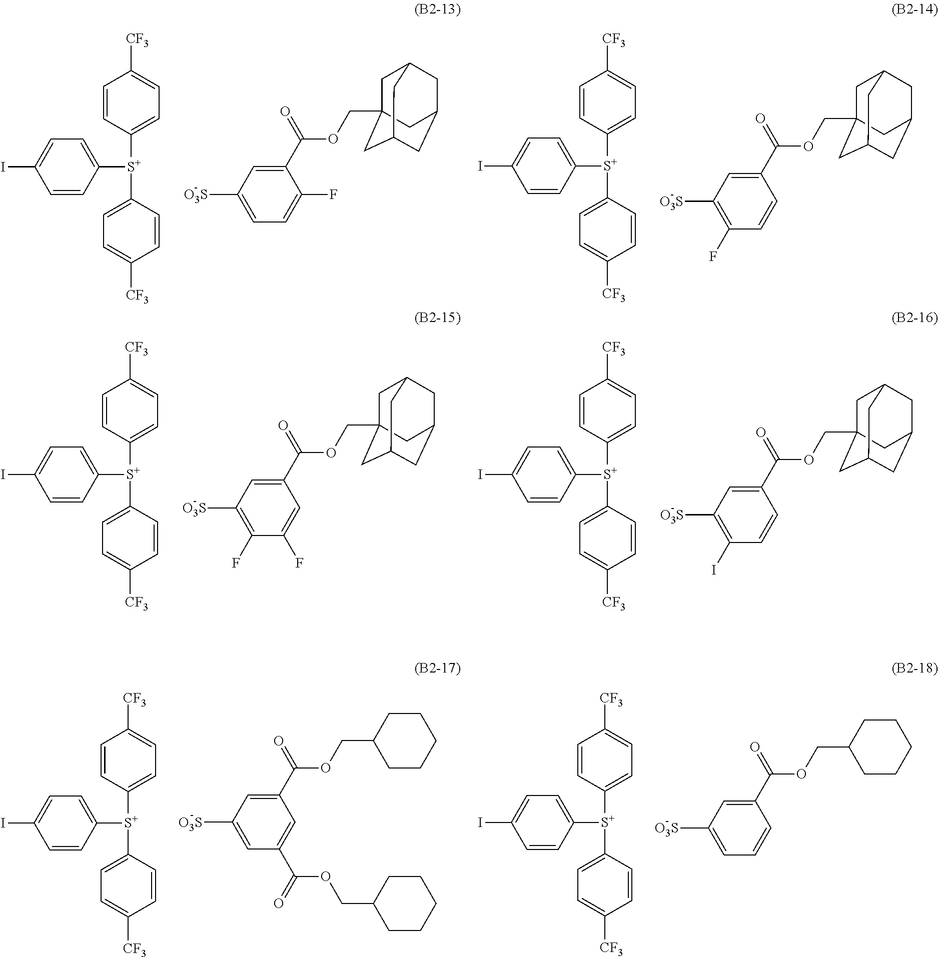

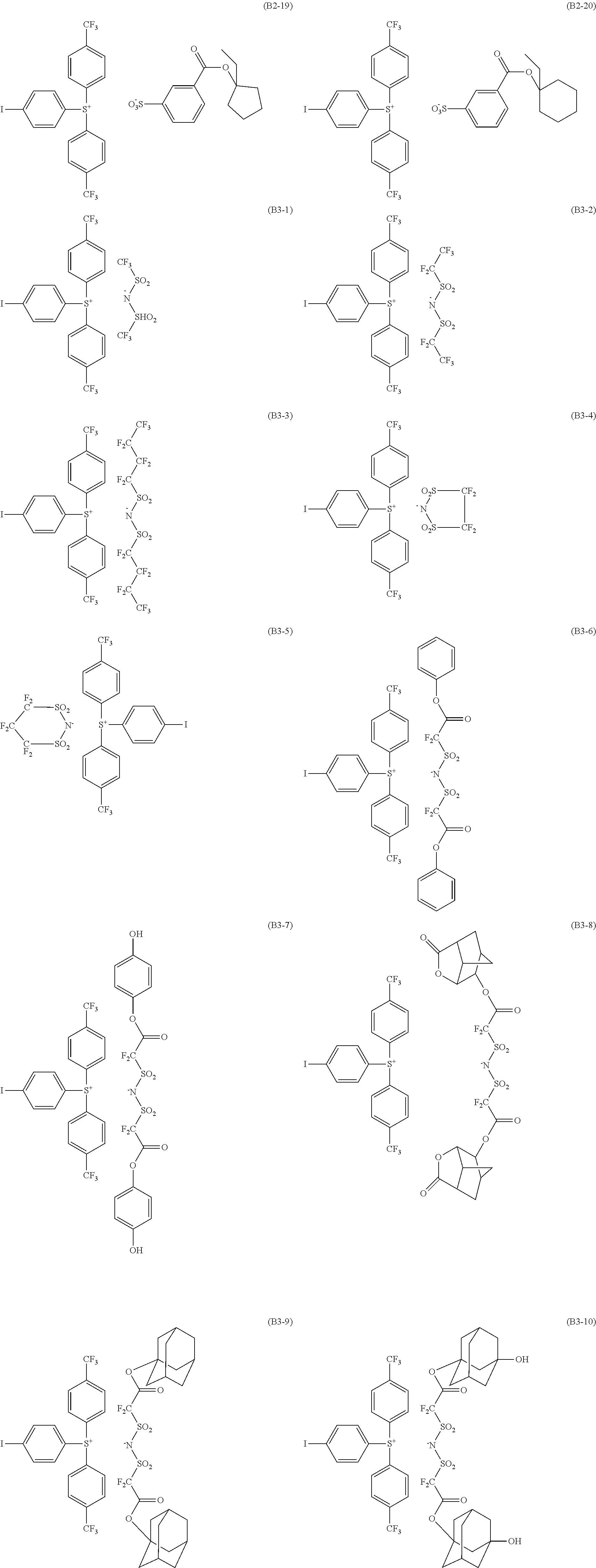

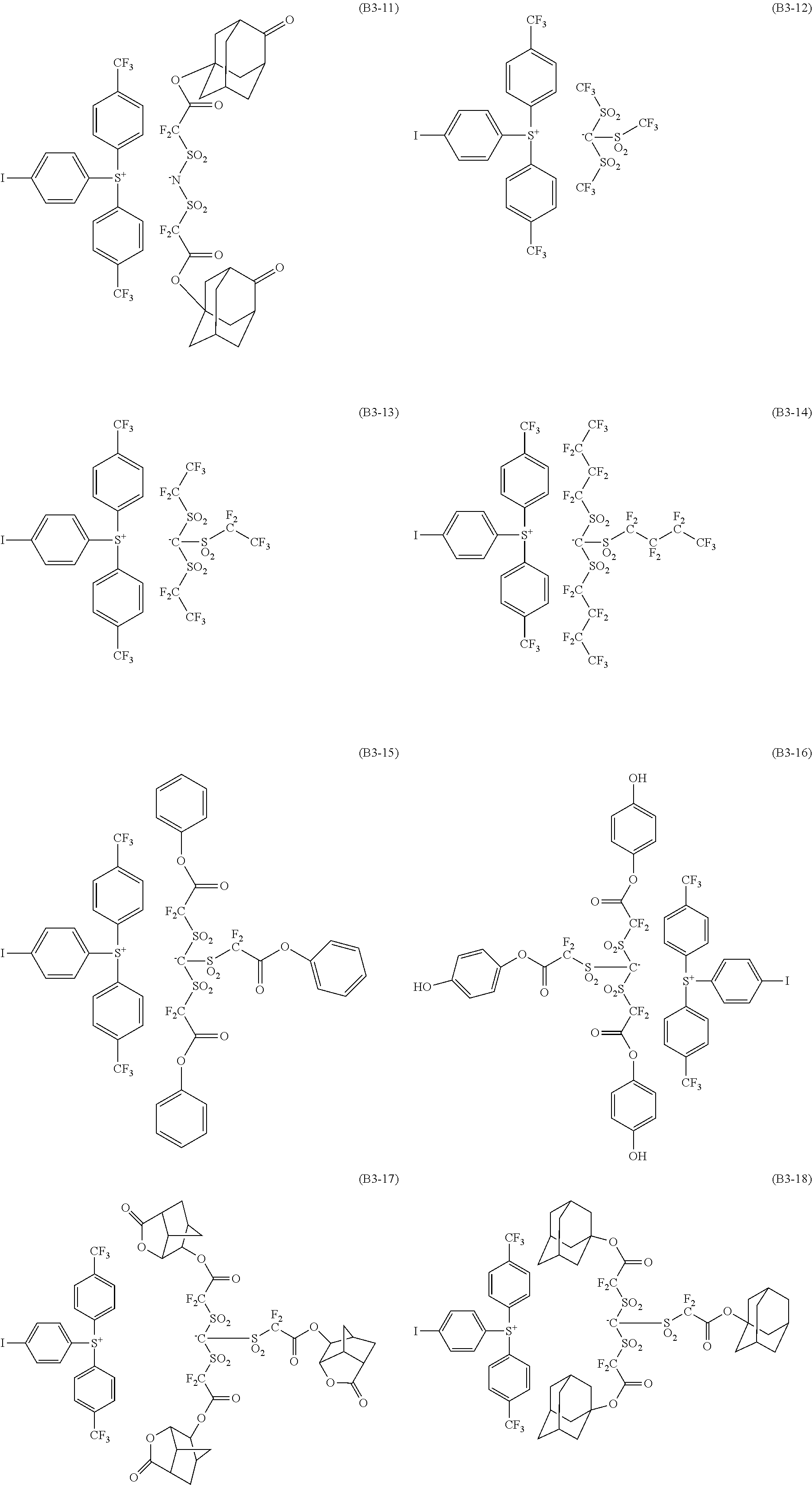

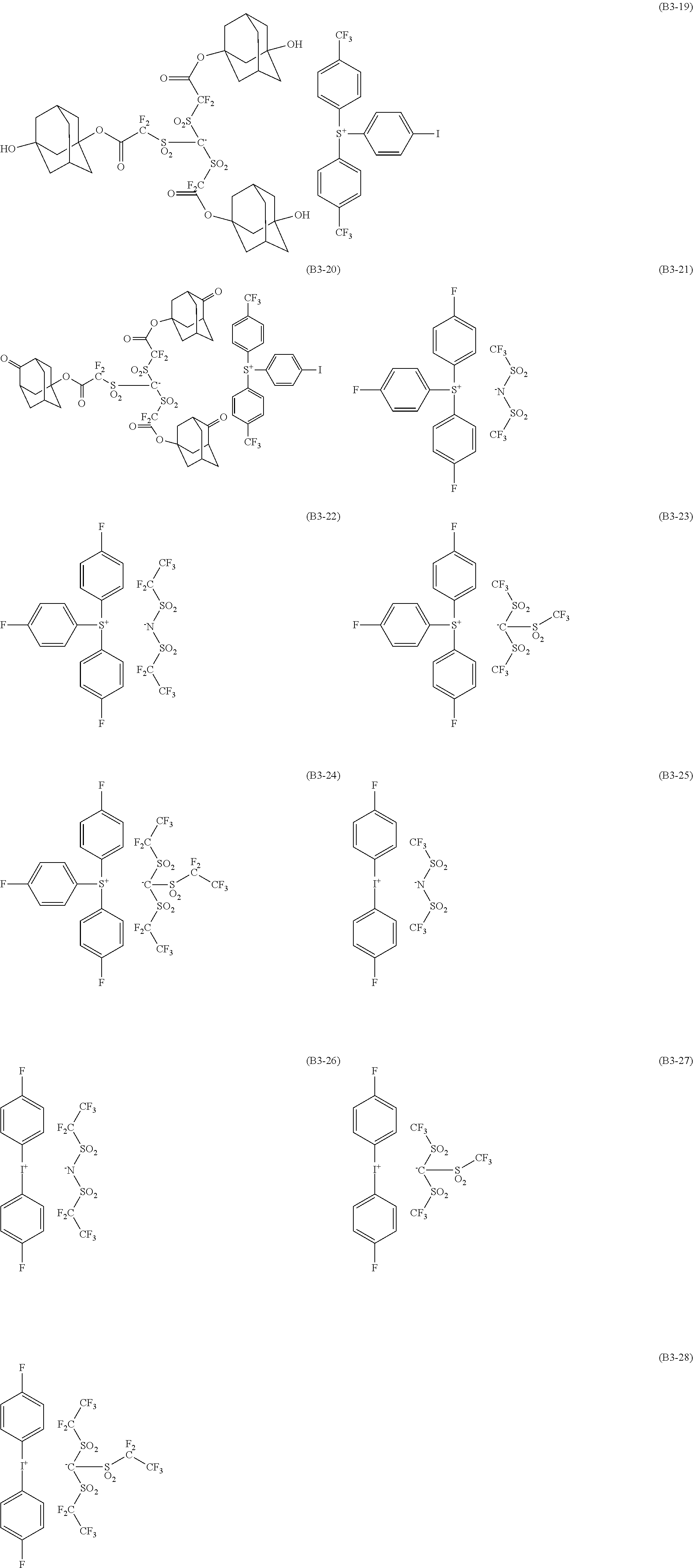

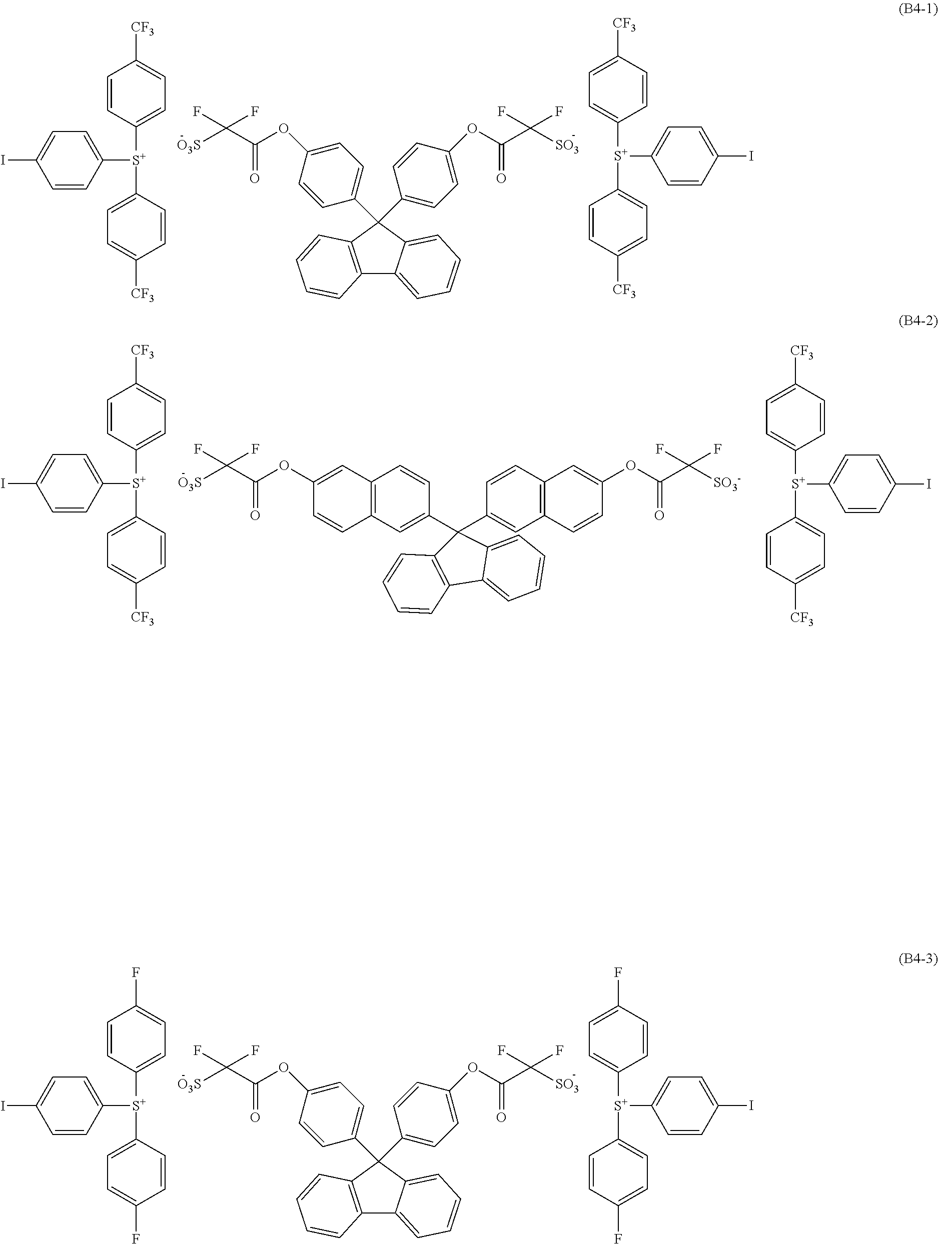

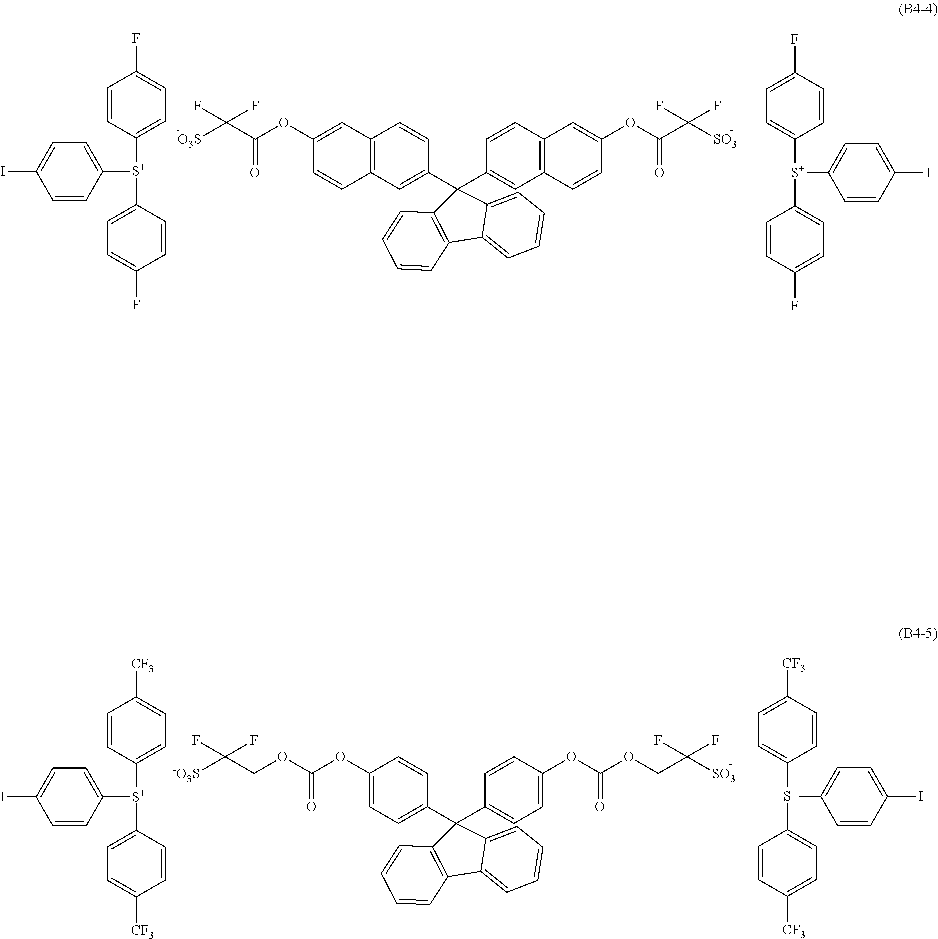

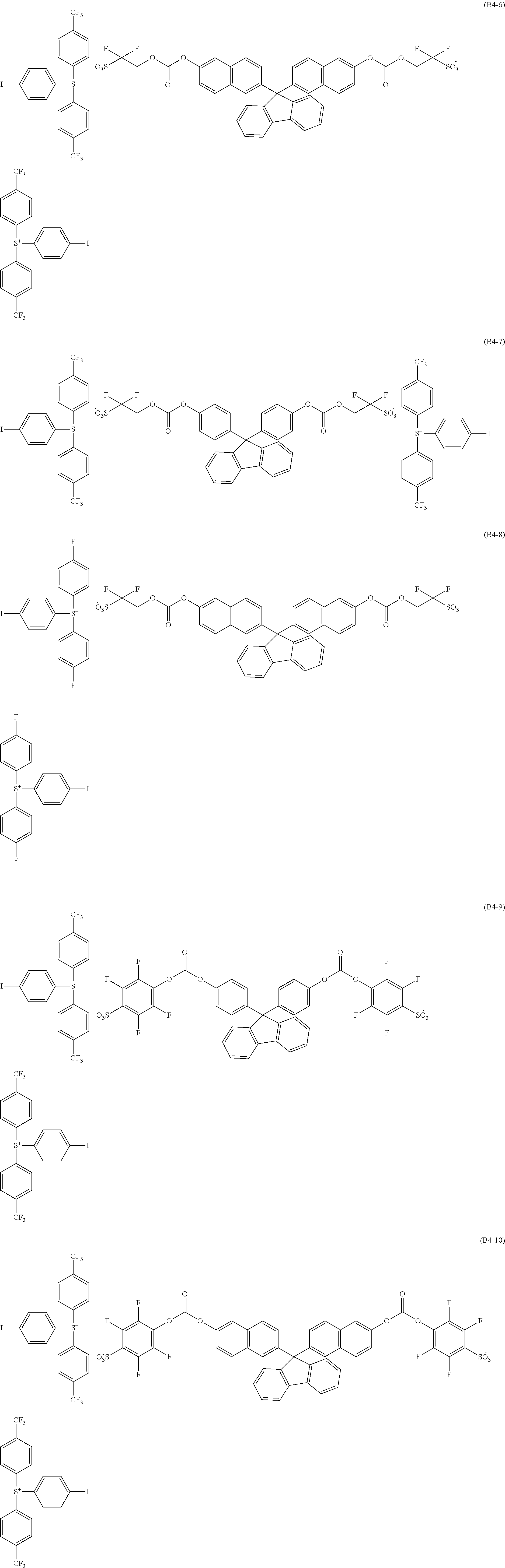

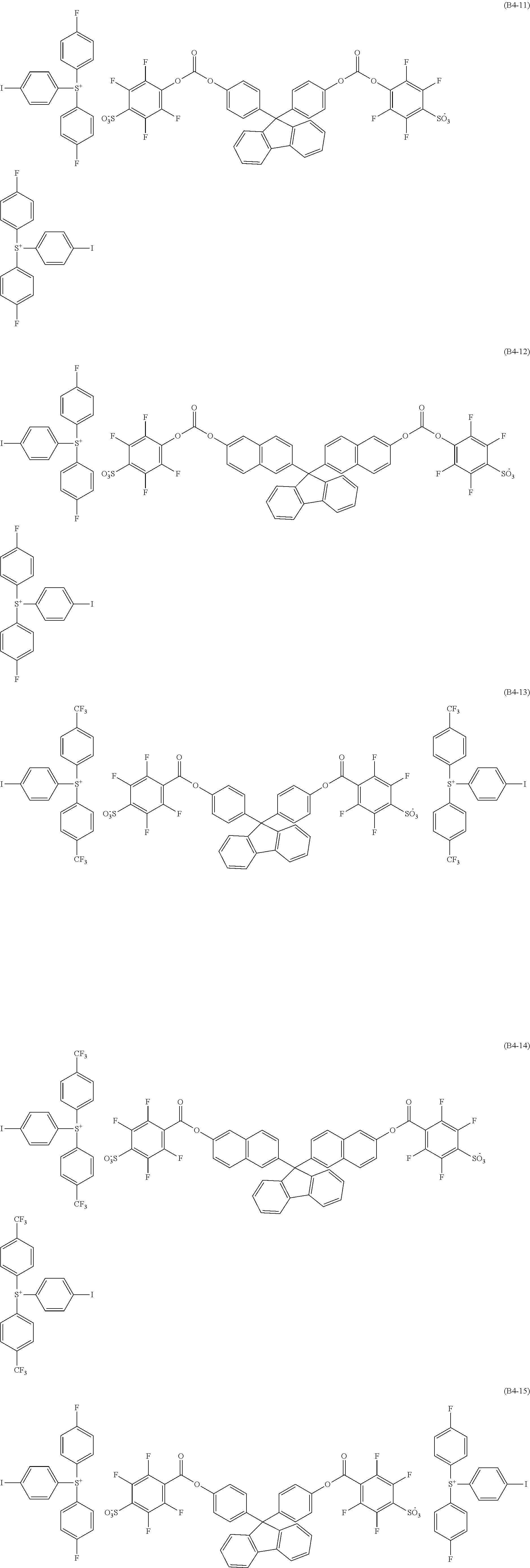

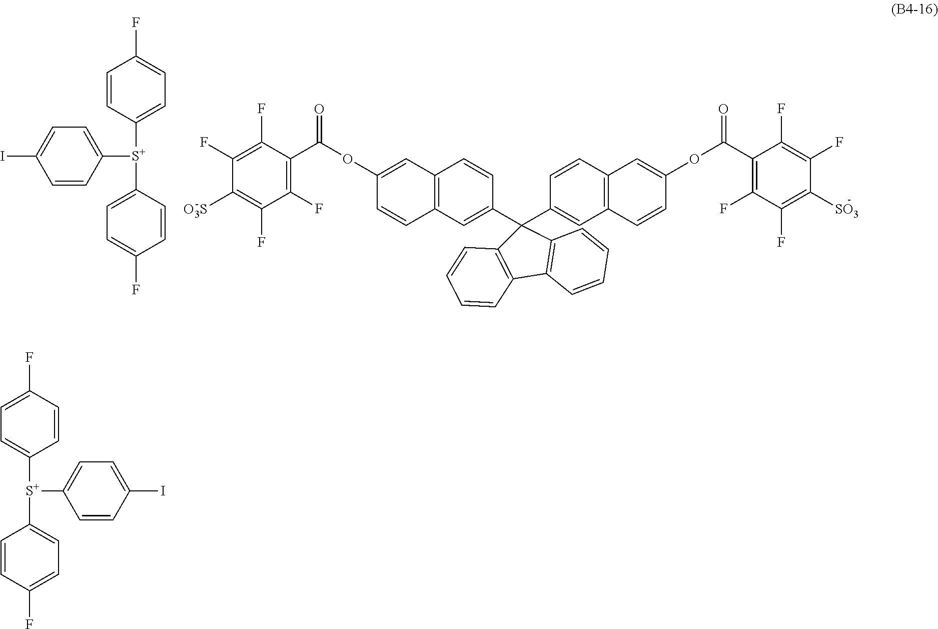

When the resist composition of the present invention includes the compound (B), the content of the compound (B) is preferably 0.1 mass % or more and 99.9 mass % or less, more preferably 1 mass % or more and 45 mass % or less, still more preferably 1 mass % or more and 40 mass % or less, and yet more preferably 2 mass % or more and 40 mass % or less, based on the solid content of the resist composition. When the resist composition includes the resin (A) or the like, the content of the compound (B) is preferably 1 part by mass or more and 45 parts by mass or less, more preferably 1 part by mass or more and 40 parts by mass or less, and still more preferably 2 parts by mass or more and 40 parts by mass or less, based on 100 parts by mass of the resin (A) or the like.

In the resist composition of the present invention, the total content of the acid generator is preferably 0.1 mass % or more and 99.9 mass % or less, more preferably 1 mass % or more and 45 mass % or less, still more preferably 1 mass % or more and 40 mass % or less, and yet more preferably 2 mass % or more and 40 mass % or less, based on the solid content of the resist composition. When including the resin (A) or the like, the total content of the acid generator is preferably 1 part by mass or more and 45 parts by mass or less, more preferably 1 part by mass or more and 40 parts by mass or less, and still more preferably 2 parts by mass or more and 40 parts by mass or less, based on 100 parts by mass of the resin (A) or the like.

### <Solvent (E)>

The content of the solvent (E) in the resist composition is typically 90 mass % or more and 99.9 mass % or less, preferably 92 mass % or more and 99 mass % or less, and more preferably 94 mass % or more and 99 mass % or less. The content of the solvent (E) can be measured, for example, by a known analysis means such as liquid chromatography or gas chromatography.

Examples of the solvent (E) include: glycol ether esters such as ethylcellosolve acetate, methylcellosolve acetate, and propylene glycol monomethyl ether acetate; glycol ethers such as propylene glycol monomethyl ether; esters such as ethyl lactate, butyl acetate, amyl acetate, and ethyl pyruvate; ketones such as acetone, methyl isobutyl ketone, 2-heptanone, and cyclohexanone; and cyclic esters such as y-butyrolactone. The solvent (E) may be used singly, or two or more solvents may be used.

### <Quencher (C)>

Examples of the quencher (C) include a basic nitrogen-containing organic compound, and a salt generating an acid having an acidity lower than that of an acid generated from an acid generator (salt (I), structural unit (IP), or compound (B)). When the resist composition includes the quencher (C), the content of the quencher (C) is preferably about 0.01 to 15 mass %, more preferably about 0.01 to 10 mass %, still more preferably about 0.1 to 8 mass %, and yet more preferably about 0.1 to 7 mass %, based on the amount of the solid content of the resist composition.

Examples of the basic nitrogen-containing organic compound include amine and an ammonium salt. Examples of the amine include an aliphatic amine and an aromatic amine. Examples of the aliphatic amine include a primary amine, a secondary amine, and a tertiary amine.

Examples of the amine include 1-naphthylamine, 2-naphthylamine, aniline, diisopropylaniline, 2-, 3-, or 4-methylaniline, 4-nitroaniline, N-methylaniline, N,N-dimethylaniline, diphenylamine, hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethyldipentylamine, ethyldihexylamine, ethyldiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane, 4,4′-diamino-3,3′-diethyldiphenylmethane, 2,2′-methylenebisaniline, imidazole, 4-methylimidazole, pyridine, 4-methylpyridine, 1,2-di(2-pyridyl)ethane, 1,2-di(4-pyridyl)ethane, 1,2-di(2-pyridyl)ethene, 1,2-di(4-pyridyl)ethene, 1,3-di(4-pyridyl)propane, 1,2-di(4-pyridyloxy)ethane, di(2-pyridyl)ketone, 4,4′-dipyridyl sulfide, 4,4′-dipyridyl disulfide, 2,2′-dipyridylamine, 2,2′-dipicolylamine, and bipyridine, preferably diisopropylaniline, and more preferably 2,6-diisopropylaniline.

Examples of the ammonium salt include tetramethylammonium hydroxide, tetraisopropylammonium hydroxide, tetrabutylammonium hydroxide, tetrahexylammonium hydroxide, tetraoctylammonium hydroxide, phenyltrimethylammonium hydroxide, 3-(trifluoromethyl)phenyltrimethylammonium hydroxide, tetra-n-butylammonium salicylate, and choline.

The acidity in a salt generating an acid having an acidity lower than that of an acid generated from the acid generator is indicated by the acid dissociation constant (pKa). For the salt generating an acid having an acidity lower than that of an acid generated from the acid generator, the acid dissociation constant of an acid generated from the salt typically meets the following inequality: −3<pKa, preferably −1<pKa<7, and more preferably 0<pKa<5.

Examples of the salt generating an acid having an acidity lower than that of an acid generated from the acid generator include salts represented by the following formulas, a salt represented by a formula (D) described in JP-A-2015-147926 (hereinafter, may be referred to as “weak acid inner salt (D)”), and salts described in JP-A-2012-229206, JP-A-2012-6908, JP-A-2012-72109, JP-A-2011-39502, and JP-A-2011-191745. The salt generating an acid having an acidity lower than that of an acid generated from the acid generator is preferably a salt generating a carboxylic acid having an acidity lower than that of an acid generated from the acid generator (salt having a carboxylic acid anion), more preferably a weak acid inner salt (D), and still more preferably a diphenyliodonium salt containing a phenyl group substituted with a carboxylic acid anion among the weak acid inner salt (D).

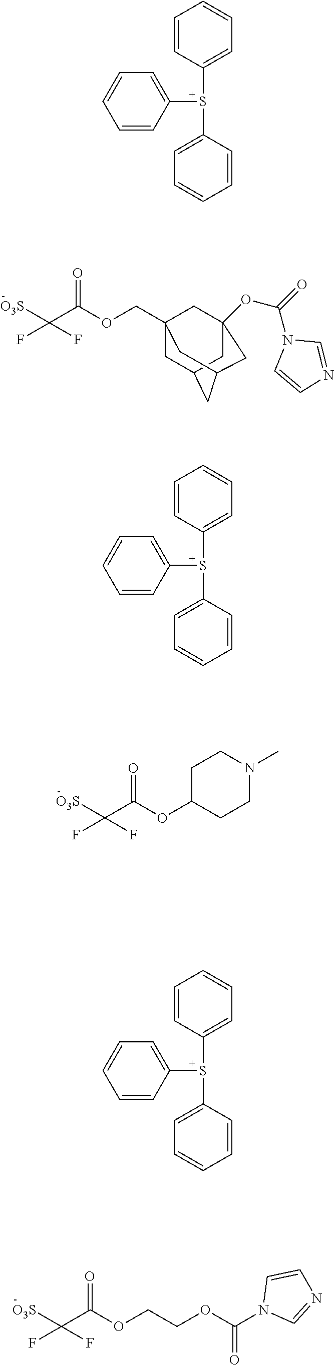

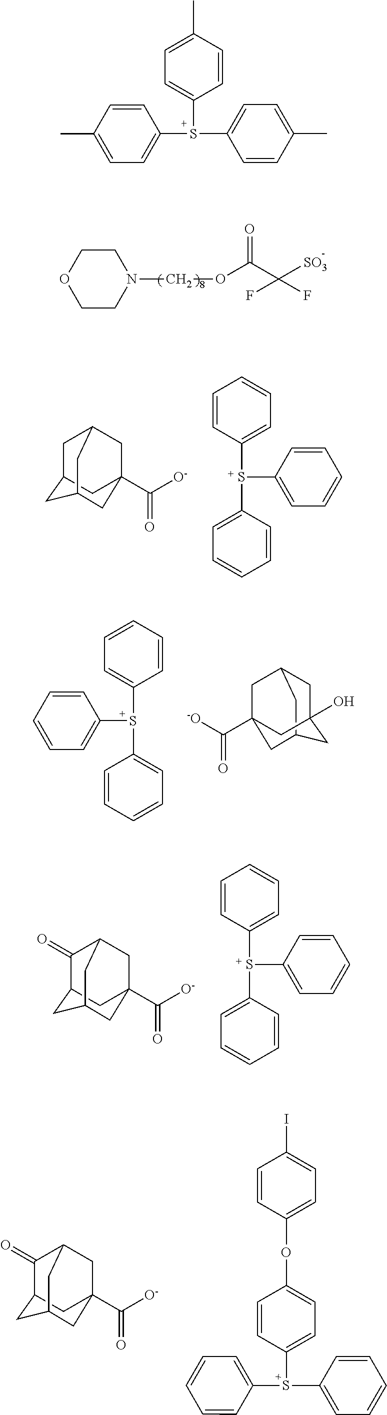

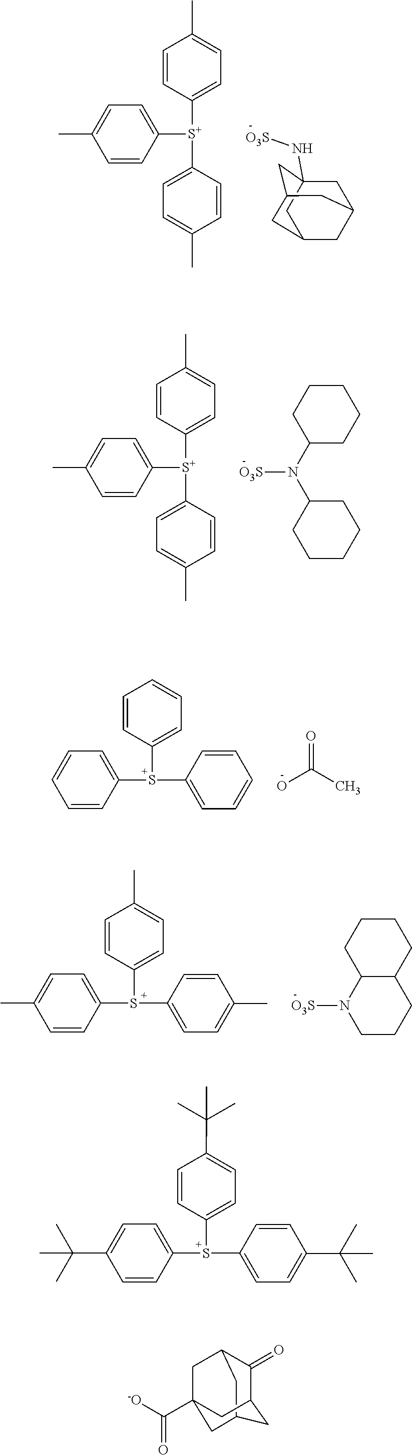

Preferable examples of the weak acid inner salt (D) include a diphenyliodonium salt having an iodonium cation to which two phenyl groups are bonded, and a carboxylic acid anion substituted with at least one phenyl group of two phenyl groups bonded to the iodonium cation, and specific examples thereof include a salt represented by the following formula.

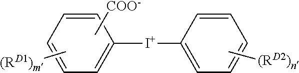

[In the formula (D),
R^{D}1 and R^{D2} each independently represent a hydrocarbon group having 1 to 12 carbon atoms, an alkoxy group having 1 to 6 carbon atoms, an acyl group having 2 to 7 carbon atoms, an acyloxy group having 2 to 7 carbon atoms, an alkoxycarbonyl group having 2 to 7 carbon atoms, a nitro group, or a halogen atom.

m′ and n′ each independently represent an integer of 0 to 4, and when m′ is 2 or more, a plurality of R^{D}1 may be the same or different, and when n′ is 2 or more, a plurality of R^{D2} may be the same or different.]

Examples of the hydrocarbon group as for R^{D1} and R^{D2} include a chain hydrocarbon group, an alicyclic hydrocarbon group, an aromatic hydrocarbon group, and groups formed by combining these groups.

Examples of the chain hydrocarbon group include alkyl groups such as a methyl group, an ethyl group, a propyl group, an isopropyl group, a butyl group, an isobutyl group, a tert-butyl group, a pentyl group, a hexyl group, and a nonyl group.

The alicyclic hydrocarbon group may be either monocyclic or polycyclic, or may be either saturated or unsaturated. Examples thereof include: cycloalkyl groups such as a cyclopropyl group, a cyclobutyl group, a cyclopentyl group, a cyclohexyl group, a cyclononyl group, and a cyclododecyl group; a norbornyl group; and an adamantyl group.

Examples of the aromatic hydrocarbon group include aryl groups such as a phenyl group, a 1-naphthyl group, a 2-naphthyl group, a 2-methylphenyl group, a 3-methylphenyl group, a 4-methylphenyl group, a 4-ethylphenyl group, a 4-propylphenyl group, a 4-isopropylphenyl group, a 4-butylphenyl group, a 4-t-butylphenyl group, a 4-hexylphenyl group, a 4-cyclohexylphenyl group, an anthryl group, a p-adamantylphenyl group, a tolyl group, a xylyl group, a cumenyl group, a mesityl group, a biphenyl group, a phenanthryl group, a 2,6-diethylphenyl group, and a 2-methyl-6-ethylphenyl group.

Examples of the groups formed by combining these groups include an alkyl-cycloalkyl group, a cycloalkyl-alkyl group, and an aralkyl group (for example, a phenylmethyl group, a 1-phenylethyl group, a 2-phenylethyl group, a 1-phenyl-1-propyl group, a 1-phenyl-2-propyl group, a 2-phenyl-2-propyl group, a 3-phenyl-1-propyl group, a 4-phenyl-1-butyl group, a 5-phenyl-1-pentyl group, a 6-phenyl-1-hexyl group, and the like).

Examples of the alkoxy group include a methoxy group and an ethoxy group.

Examples of the acyl group include an acetyl group, a propanoyl group, a benzoyl group, and a cyclohexanecarbonyl group.

Examples of the acyloxy group include groups obtained by bonding an oxy group (—O—) to the above acyl group.

Examples of the alkoxycarbonyl group include groups obtained by bonding a carbonyl group (—CO—) to the above alkoxy group.

Examples of the halogen atom include a fluorine atom, a chlorine atom, a bromine atom, and an iodine atom.

Preferably, R^{D}1 and R^{D2} each independently represent an alkyl group having 1 to 8 carbon atoms, a cycloalkyl group having 3 to 10 carbon atoms, an alkoxy group having 1 to 6 carbon atoms, an acyl group having 2 to 4 carbon atoms, an acyloxy group having 2 to 4 carbon atoms, an alkoxycarbonyl group having 2 to 4 carbon atoms, a nitro group, or a halogen atom.

Preferably, m′ and n′ are each independently an integer of 0 to 2, and more preferably 0. When m′ is 2 or more, a plurality of R^{D1} may be the same or different, and when n′ is 2 or more, a plurality of R^{D2} may be the same or different.

More specifically, the following salts are exemplified.

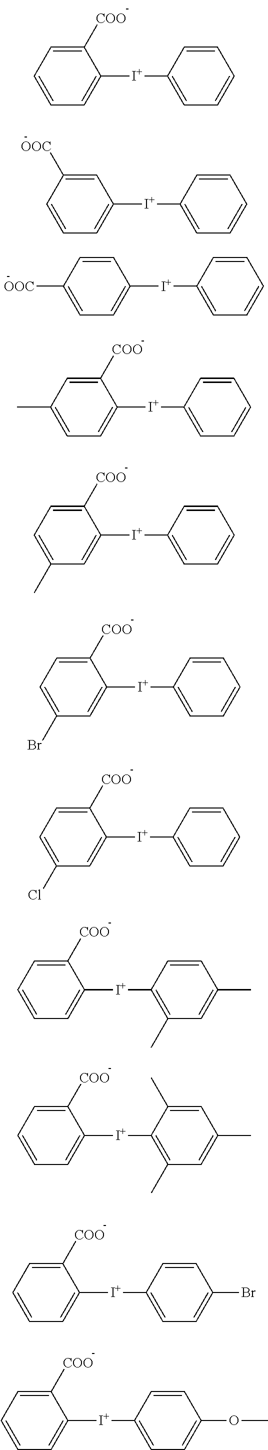

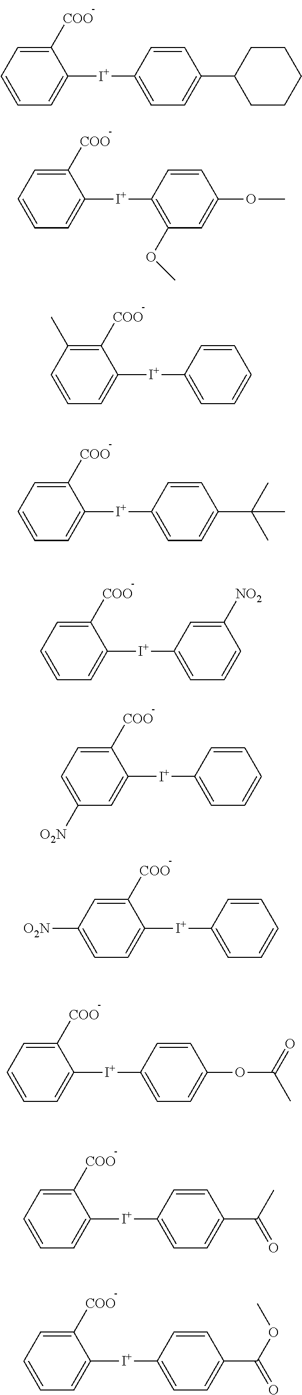

### <Other Components>

The resist composition of the present invention may also include components other than the components described above (hereinafter, may be referred to as “other components (F)”). The other components (F) are not particularly limited and it is possible to use various additives known in the resist field, for example, sensitizers, dissolution inhibitors, surfactants, stabilizers, and dyes.

### <Preparation of Resist Composition>

The resist composition of the present invention can be prepared by mixing the salt (I) or an acid generator including the structural unit (IP) and, if necessary, the resin (A) or the like, the compound (B), resins other than the resin (A) or the like, the solvent (E), the quencher (C), and other components (F). The order of mixing these components is any order and is not particularly limited. It is possible to select, as the temperature during mixing, appropriate temperature from 10 to 40° C., according to the type of the resin and the solubility in the solvent (E) of the resin and the like. It is possible to select, as the mixing time, appropriate time from 0.5 to 24 hours according to the mixing temperature. The mixing means is not particularly limited and it is possible to use mixing with stirring.

After mixing the respective components, the mixture is preferably filtered through a filter having a pore size of about 0.003 to 0.2 μm.

### <Method for Producing Resist Pattern>

The method for producing a resist pattern of the present invention includes:
(1) applying the resist composition of the present invention on a substrate;
(2) drying the applied composition to form a composition layer; (3) exposing the composition layer;
(4) heating the exposed composition layer; and
(5) developing the heated composition layer.

The resist composition can be typically applied on a substrate using a conventionally used apparatus, such as a spin coater. Examples of the substrate include inorganic substrates such as a silicon wafer, and organic substrates and the like in which a resist film or the like is formed on the surface. Before applying the resist composition, the substrate may be washed, and an antireflection film may be formed on the substrate.

The solvent is removed by drying the applied composition to form a composition layer. Drying is performed by evaporating the solvent using a heating device such as a hot plate (so-called “prebake”), or using a decompression device. The heating temperature is preferably 50 to 200° C. and the heating time is preferably 10 to 180 seconds. The pressure during drying under reduced pressure is preferably about 1 to 1.0×10⁵ Pa.

The composition layer thus obtained is typically exposed using an aligner. The aligner may be a liquid immersion aligner. It is possible to use, as an exposure source, various exposure sources, for example, exposure sources capable of emitting laser beam in an ultraviolet region such as KrF excimer laser (wavelength of 248 nm), ArF excimer laser (wavelength of 193 nm) and F₂ excimer laser (wavelength of 157 nm), an exposure source capable of emitting harmonic laser beam in a far-ultraviolet or vacuum ultraviolet region by wavelength-converting laser beam from a solid-state laser source (YAG or semiconductor laser), and an exposure source capable of emitting electron beam or extreme ultraviolet light (EUV). As used herein, such exposure to radiation may be collectively referred to as “exposure”. The exposure is typically performed through a mask corresponding to a pattern to be required. When electron beam is used as the exposure source, exposure may be performed by direct writing without using the mask.

The exposed composition layer is subjected to a heat treatment (so-called “post-exposure bake”) to promote the deprotection reaction in an acid-labile group. The heating temperature is typically approximately 50 to 200° C., and preferably about 70 to 150° C. It is also possible to perform a chemical treatment (silylation) that adjusts the hydrophilicity or hydrophobicity of the resin on a surface side of the composition after heating. In addition, before performing the development, the steps of application of the resist composition, drying, exposure, and heating may be repeatedly performed on the exposed composition layer.

The heated composition layer is typically developed with a development solution using a development apparatus. Examples of the development method include a dipping method, a paddle method, a spraying method, and a dynamic dispensing method. The development temperature is preferably, for example, 5 to 60° C. and the development timc is preferably, for example, 5 to 300 seconds. It is possible to produce a positive resist pattern or negative resist pattern by selecting the type of the development solution as follows.

When the positive resist pattern is produced from the resist composition of the present invention, an alkaline development solution is used as the development solution. The alkaline development solution may be various aqueous alkaline solutions used in this field. Examples thereof include aqueous solutions of tetramethylammonium hydroxide and (2-hydroxyethyl)trimethylammonium hydroxide (commonly known as choline). The surfactant may be included in the alkaline development solution.

It is preferable that the developed resist pattern is washed with ultrapure water and then water remaining on the substrate and the pattern is removed.

When the negative resist pattern is produced from the resist composition of the present invention, a development solution including an organic solvent (hereinafter, may be referred to as “organic development solution”) is used as the development solution.

Examples of the organic solvent included in the organic development solution include ketone solvents such as 2-hexanone and 2-heptanone; glycol ether ester solvents such as propylene glycol monomethyl ether acetate; ester solvents such as butyl acetate; glycol ether solvents such as propylene glycol monomethyl ether; amide solvents such as N,N-dimethylacetamide; and aromatic hydrocarbon solvents such as anisole.

The content of the organic solvent in the organic development solution is preferably 90 mass % or more and 100 mass % or less, more preferably 95 mass % or more and 100 mass % or less, and still more preferably the organic development solution is substantially composed of the organic solvent.

Particularly, the organic development solution is preferably a development solution including butyl acetate and/or 2-heptanone. The total content of butyl acetate and 2-heptanone in the organic development solution is preferably 50 mass % or more and 100 mass % or less, more preferably 90 mass % or more and 100 mass % or less, and still more preferably the organic development solution is substantially composed of butyl acetate and/or 2-heptanone.

The surfactant may be included in the organic development solution. A trace amount of water may be included in the organic development solution.

During development, the development may be stopped by substituting by a solvent with the type different from that of the organic development solution.

The developed resist pattern is preferably washed with a rinsing solution. The rinsing solution is not particularly limited as long as it does not dissolve the resist pattern, and it is possible to use a solution including an ordinary organic solvent that is preferably an alcohol solvent or an ester solvent.

After washing, the rinsing solution remaining on the substrate and the pattern is preferably removed.

### <Applications>

The resist composition of the present invention is suitable as a resist composition for exposure of KrF excimer laser, a resist composition for exposure of ArF excimer laser, a resist composition for exposure of electron beam (EB) or a resist composition for exposure of EUV, particularly a resist composition for exposure of electron beam (EB) or a resist composition for exposure of EUV, and the resist composition is useful for fine processing of semiconductors.

### EXAMPLES

The present invention will be described more specifically by way of Examples. Percentages and parts expressing the contents or amounts used in the Examples are by mass unless otherwise specified.

The weight-average molecular weight is a value determined by gel permeation chromatography. Analysis conditions of gel permeation chromatography are as follows.
Column: TSKgel Multipore HXL-M×3+guardcolumn (manufactured by TOSOH CORPORATION)
Eluent: tetrahydrofuran
Flow rate: 1.0 mL/min
Detector: RI detector
Column temperature: 40° C.
Injection amount: 100 μl
Molecular weight standards: polystyrene standard (manufactured by TOSOH CORPORATION)

Structures of compounds were confirmed by measuring a molecular ion peak using mass spectrometry (LC is Model 1100, manufactured by Agilent Technologies, Inc., and MASS is Model LC/MSD, manufactured by Agilent Technologies, Inc.). The value of this molecular ion peak in the following Examples is indicated by “MASS”.

### Example 1: Synthesis of Salt Represented by Formula (I-9)

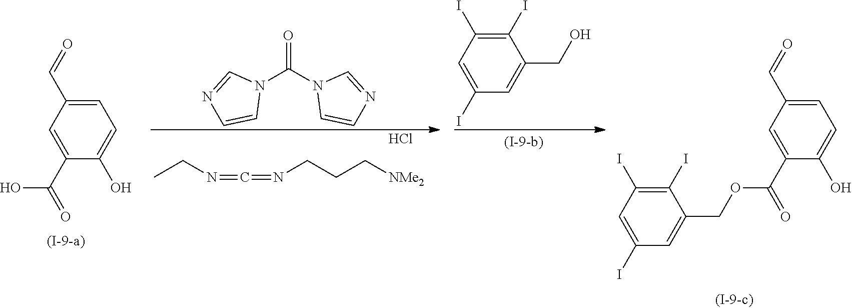

1.66 parts of a compound represented by a formula (I-9-a) and 20 parts of tetrahydrofuran were added, and the mixture was stirred at 23° C. for 30 minutes, and then charged with 1.62 parts of carbonyldiimidazole and 1.92 parts of 1-[3-(dimethylamino) propyl]-3-ethylcarbodiimide hydrochloride, and the mixture was stirred at 23° C. for 1 hour and then further stirred at 50° C. for 2 hours. 4.86 parts of a compound represented by a formula (I-9-b) were added to the resulting reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm; development solvent: n-heptane/ethyl acetate=3/1, manufactured by KANTO CHEMICAL CO., INC.) to obtain 4.50 parts of a compound represented by a formula (I-9-c).

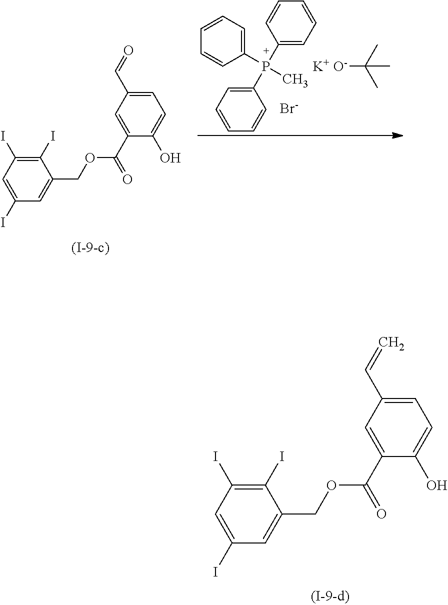

1.79 parts of methyltriphenylphosphonium bromide, 1.12 parts of potassium t-butoxide and 40 parts of tetrahydrofuran were mixed, and the mixture was stirred at 23° C. for 1 hour. While cooling the obtained reaction product to 10° C., 3.17 parts of a compound represented by a formula (I-9-c) were added, then the temperature was raised to 23° C., and the mixture was stirred for 16 hours. While the obtained reaction product was cooled to 10° C., 2.24 parts of a 5% oxalic acid aqueous solution were added and stirred for 30 minutes. 40 parts of ethyl acetate and 20 parts of ion-exchanged water were added to the obtained reaction product, and the mixture was stirred at 23° C. for 30 minutes, then separated, and an organic layer was taken out. This water washing operation was repeated five times. The organic layer thus obtained was concentrated and then the concentrated mixture was isolated using a column (silica gel 60N (spherical, neutral) 100 to 210 μm, manufactured by KANTO CHEMICAL CO. INC., development solvent: n-heptane/ethyl acetate=10/1) to obtain 2.69 parts of a compound represented by a formula (I-9-d).

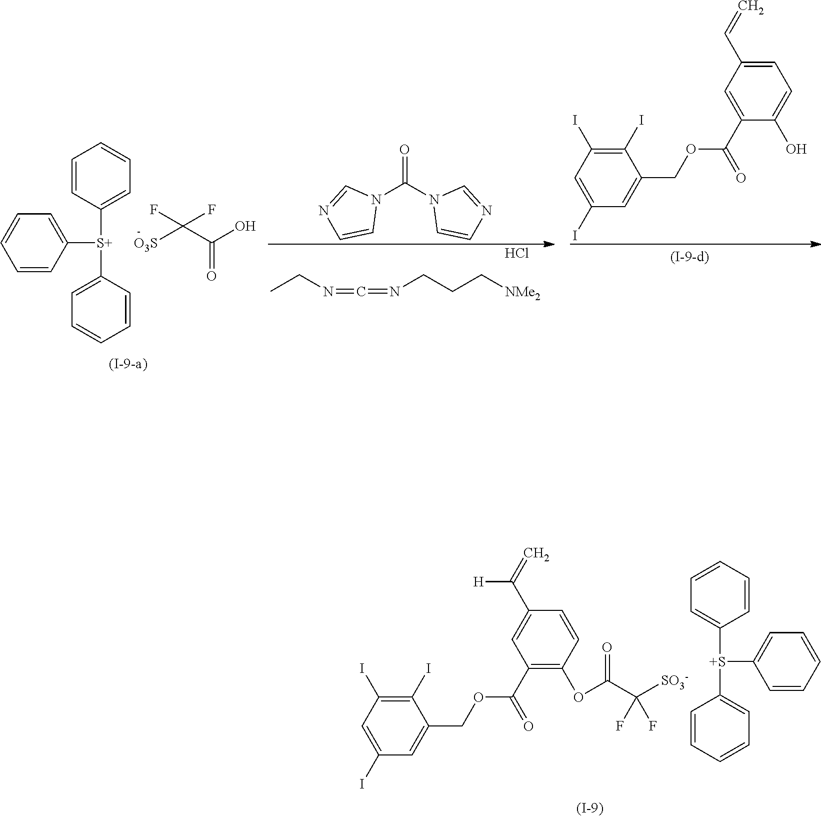

1.75 parts of a salt represented by a formula (I-9-e) and 25 parts of acetonitrile were added, and the mixture was stirred at 23° C. for 30 minutes, and then charged with 0.65 parts of carbonyldiimidazole and 0.77 parts of 1-[3-(dimethylamino)propyl]-3-ethylcarbodiimide hydrochloride, and the mixture was stirred at 23° C. for 1 hour and then further stirred at 50° C. for 2 hours. 2.53 parts of a compound represented by a formula (I-9-d) were added to the obtained reaction product, and the mixture was further stirred at 50° C. for 2 hours and then cooled to 23° C. 50 parts of chloroform and 30 parts of ion-exchanged water were added to the obtained reaction mass, and the mixture was stirred at 23° C. for 30 minutes, then separated, and an organic layer was taken out. This water washing operation was repeated four times. The organic layer thus obtained was concentrated and then 3 parts of acetonitrile and 30 parts of tert-butyl methyl ether were added to the concentrated residue and, after stirring at 23° C. for 30 minutes, the supernatant was removed, followed by concentration to obtain 3.12 parts of a salt represented by a formula (I-9).

MS (ESI (+) Spectrum): M+263.1

MS (ESI (−) Spectrum): M−788.7

### Example 2: Synthesis of Salt Represented by Formula (I-12)

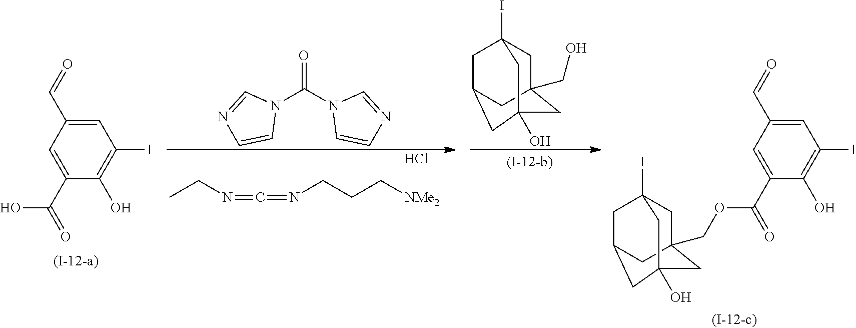

2.92 parts of a compound represented by a formula (I-12-a) and 20 parts of tetrahydrofuran were added, and the mixture was stirred at 23° C. for 30 minutes, and then charged with 1.62 parts of carbonyldiimidazole and 1.92 parts of 1-[3- (dimethylamino) propyl]-3-ethylcarbodiimide hydrochloride, and the mixture was stirred at 23° C. for 1 hour and then further stirred at 50° C. for 2 hours. 3.08 parts of a compound represented by a formula (I-12-b) were added to the obtained reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm, manufactured by KANTO CHEMICAL CO., INC.; development solvent: n-heptane/ethyl acetate=3/1) to obtain 4.07 parts of a compound represented by a formula (I-12-c).

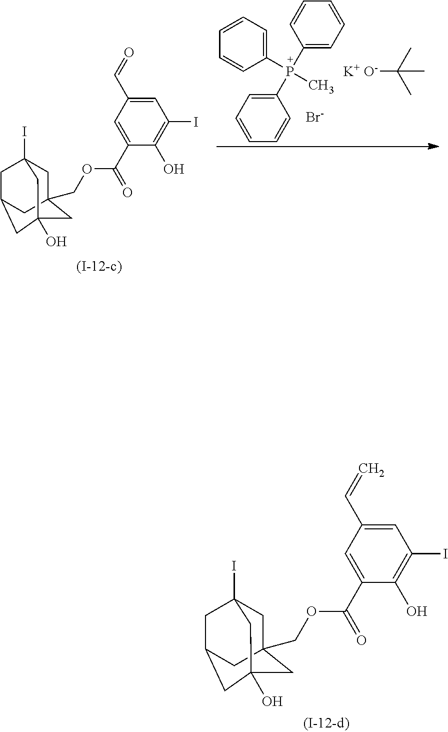

2.14 parts of methyltriphenylphosphonium bromide, 1.34 parts of potassium t-butoxide and 40 parts of tetrahydrofuran were mixed, and the mixture was stirred at 23° C. for 1 hour. While cooling the obtained reaction product to 10° C., 3.49 parts of a compound represented by a formula (I-12-c) were added, then the temperature was raised to 23° C., and the mixture was stirred for 16 hours. While the obtained reaction product was cooled to 10° C., 1.34 parts of a 5% oxalic acid aqueous solution were added and stirred for 30 minutes. 40 parts of ethyl acetate and 20 parts of ion-exchanged water were added to the obtained reaction product, and the mixture was stirred at 23° C. for 30 minutes, then separated, and an organic layer was taken out. This water washing operation was repeated five times. The organic layer thus obtained was concentrated and then the concentrated mixture was isolated using a column (silica gel 60N (spherical, neutral) 100 to 210 μm, manufactured by KANTO CHEMICAL CO. INC., development solvent: n-heptane/ethyl acetate=10/1) to obtain 2.78 parts of a compound represented by a formula (I-12-d).

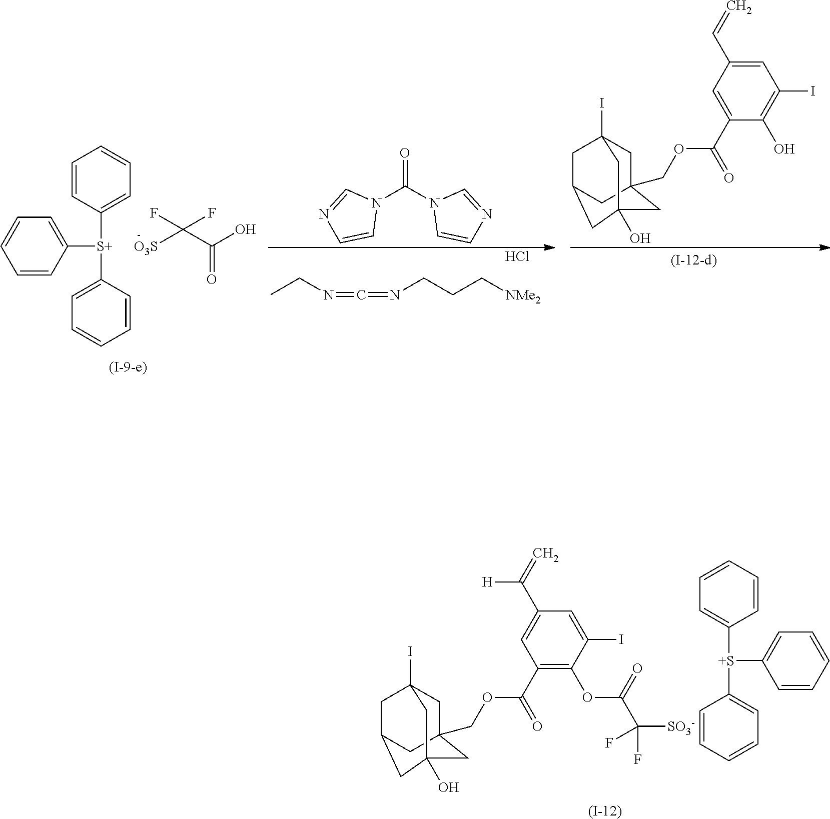

1.75 parts of a salt represented by a formula (I-9-e) and 25 parts of acetonitrile were added, and the mixture was stirred at 23° C. for 30 minutes, and then charged with 0.65 parts of carbonyldiimidazole and 0.77 parts of 1-[3-(dimethylamino)propyl]-3-ethylcarbodiimide hydrochloride, and the mixture was stirred at 23° C. for 1 hour and then further stirred at 50° C. for 2 hours. 2.32 parts of a compound represented by a formula (I-12-d) were added to the obtained reaction product, and the mixture was further stirred at 50° C. for 2 hours and then cooled to 23° C. 50 parts of chloroform and 30 parts of ion-exchanged water were added to the obtained reaction mass, and the mixture was stirred at 23° C. for 30 minutes, then separated, and an organic layer was taken out. This water washing operation was repeated four times. The organic layer thus obtained was concentrated, and then 3 parts of acetonitrile and 30 parts of tert-butyl methyl ether were added to the concentrated residue and, after stirring at 23° C. for 30 minutes, the supernatant was removed, followed by concentration to obtain 2.92 parts of a salt represented by formula (I-12).

MS (ESI (+) Spectrum): M+263.1

MS (ESI (−) Spectrum): M−736.9

### Example 3: Synthesis of Salt Represented by Formula (I-29)

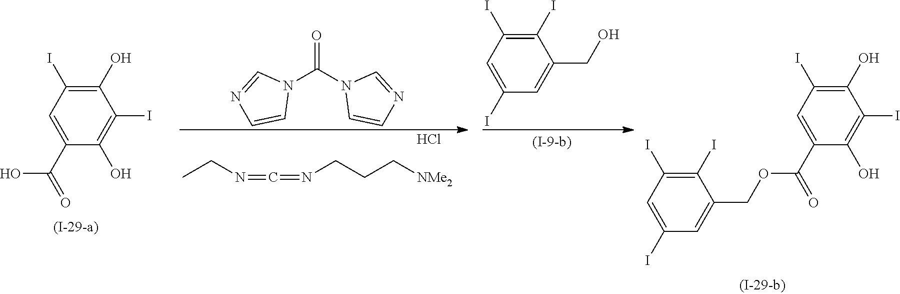

4.06 parts of a compound represented by a formula (1-29-a) and 20 parts of tetrahydrofuran were added, and the mixture was stirred at 23° C. for 30 minutes, and then charged with 1.62 parts of carbonyldiimidazole and 1.92 parts of 1-[3- (dimethylamino) propyl]-3-ethylcarbodiimide hydrochloride, and the mixture was stirred at 23° C. for 1 hour and then further stirred at 50° C. for 2 hours. 4.85 parts of a compound represented by a formula (I-9-b) were added to the obtained reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm; development solvent: n-heptane/ethyl acetate=3/1, manufactured by KANTO CHEMICAL CO., INC.) to obtain 6.20 parts of a compound represented by a formula (I-29-b).

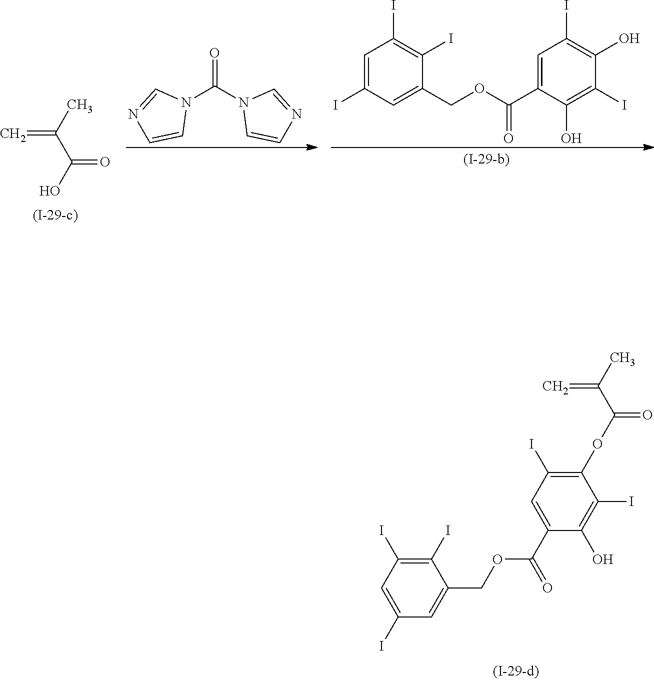

0.52 parts of a compound represented by a formula (1-29-c) and 20 parts of tetrahydrofuran were added, and the mixture was stirred at 23° C. for 30 minutes, then charged with 0.97 parts of carbonyldiimidazole, and stirred at 23° C. for 1 hour, then further stirred at 50° C. for 2 hours. 5.24 parts of a compound represented by a formula (I-29-b) were added to the obtained reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm; development solvent: n-heptane/ethyl acetate=3/1, manufactured by KANTO CHEMICAL CO., INC.) to obtain 4.41 parts of a compound represented by a formula (I-29-d).

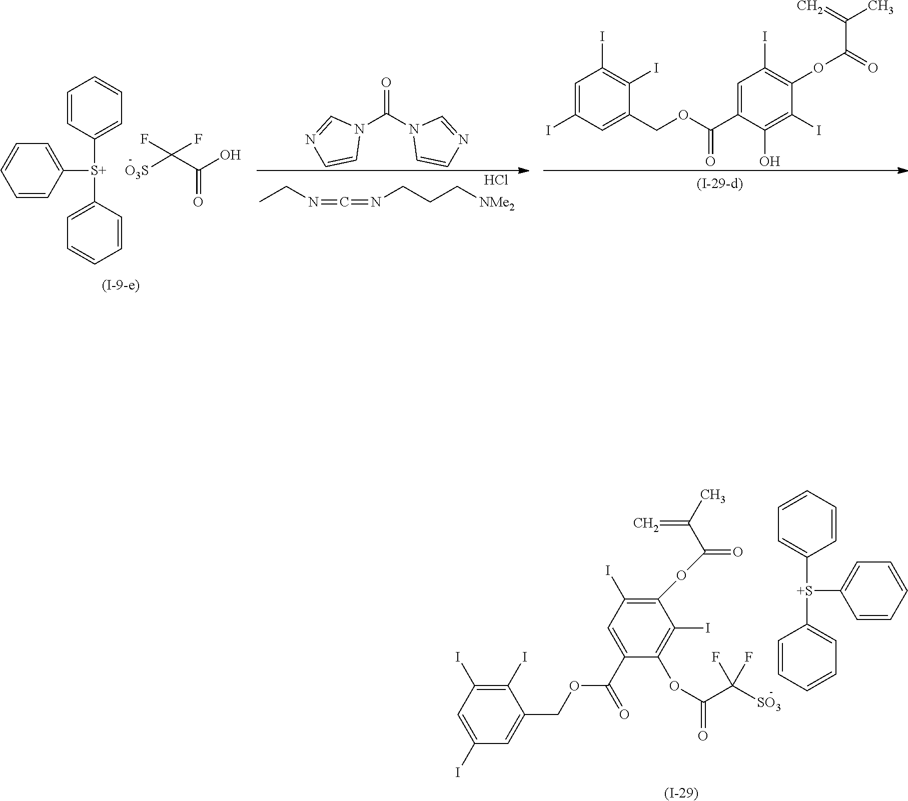

1.75 parts of a salt represented by a formula (I-9-e) and 25 parts of acetonitrile were added, and the mixture was stirred at 23° C. for 30 minutes, and then charged with 0.65 parts of carbonyldiimidazole and 0.77 parts of 1-[3-(dimethylamino)propyl]-3-ethylcarbodiimide hydrochloride, and the mixture was stirred at 23° C. for 1 hour and then further stirred at 50° C. for 2 hours. 3.77 parts of a compound represented by a formula (I-29-d) were added to the obtained reaction product, and the mixture was further stirred at 50° C. for 2 hours and then cooled to 23° C. 50 parts of chloroform and 30 parts of ion-exchanged water were added to the obtained reaction mass, and the mixture was stirred at 23° C. for 30 minutes, then separated, and an organic layer was taken out. This water washing operation was repeated four times. The organic layer thus obtained was concentrated and then 3 parts of acetonitrile and 30 parts of tert-butyl methyl ether were added to the concentrated residue and, after stirring at 23° C. for 30 minutes, the supernatant was removed, followed by concentration to obtain 4.09 parts of a salt represented by a formula (I-29).

MS (ESI (+) Spectrum): M+263.1

MS (ESI (−) Spectrum): M−1098.5

### Example 4: Synthesis of Salt Represented by Formula (I-1151)

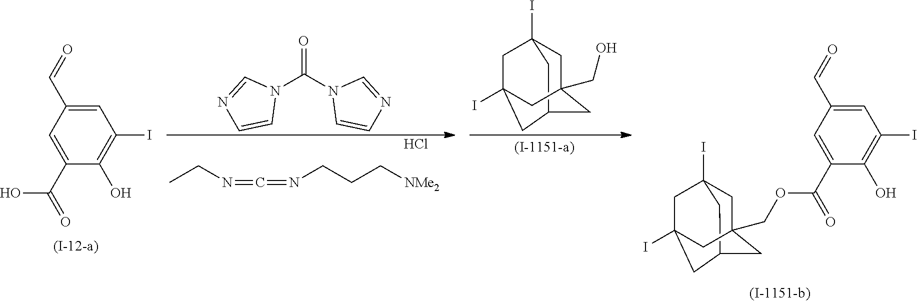

2.92 parts of a compound represented by a formula (I-12-a) and 20 parts of tetrahydrofuran were added, and the mixture was stirred at 23° C. for 30 minutes, and then charged with 1.62 parts of carbonyldiimidazole and 1.92 parts of 1-[3- (dimethylamino) propyl]-3-ethylcarbodiimide hydrochloride, and the mixture was stirred at 23° C. for 1 hour and then further stirred at 50° C. for 2 hours. 4.18 parts of a compound represented by a formula (I-1151-a) were added to the obtained reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm; development solvent: n-heptane/ethyl acetate=3/1, manufactured by KANTO CHEMICAL CO., INC.) to obtain 5.19 parts of a compound represented by a formula (1-1151-b).

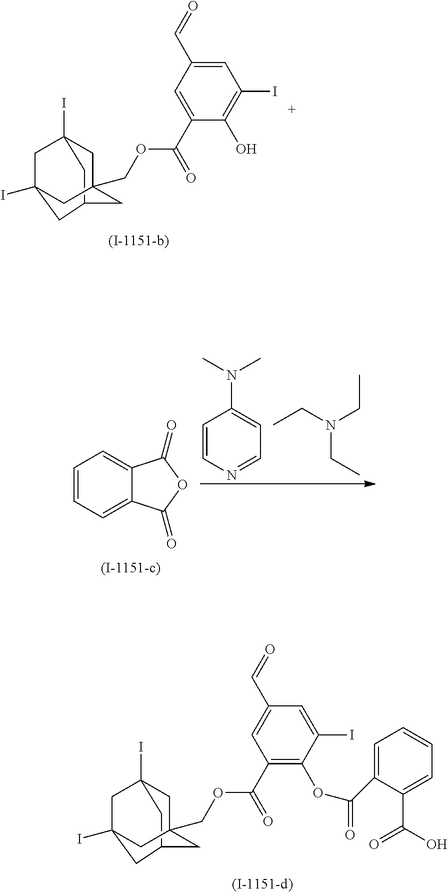

4.84 parts of a compound represented by a formula (I-1151-b), 0.94 parts of dimethylaminopyridine, 0.85 parts of triethylamine, and 40 parts of dimethylformamide were mixed, and the mixture was stirred at 23° C. for 30 minutes, then, 1.24 parts of a compound represented by a formula (I-1151-c) and 20 parts of chloroform were added thereto, and the mixture was stirred at 50° C. for 5 hours, and then further stirred at 23° C. for 2 hours. 10 parts of 1N hydrochloric acid were added to the obtained reaction mixture, and the mixture was stirred at 23° C. for 30 minutes and separated to obtain an organic layer. 30 parts of ion-exchanged water were added to the collected organic layer, and the mixture was stirred at 23° C. for 30 minutes and separated to obtain an organic layer. This water washing operation was performed three times. The organic layer thus obtained was concentrated and then the concentrated mixture was isolated using a column (silica gel 60N (spherical, neutral) 100-210 μm; manufactured by Kanto Chemical Co., Inc., developing solvent: ethyl acetate) to obtain 4.35 parts of a compound represented by formula (I-1151-d).

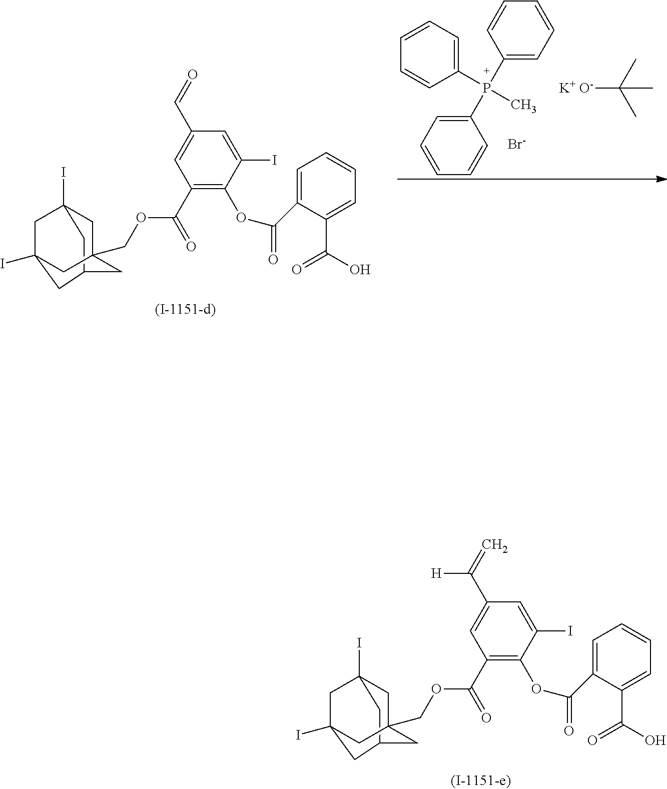

1.79 parts of methyltriphenylphosphonium bromide, 1.12 parts of potassium t-butoxide and 40 parts of tetrahydrofuran were mixed, and the mixture was stirred at 23° C. for 1 hour. While cooling the obtained reaction product to 10° C., 4.20 parts of a compound represented by a formula (1-1151-d) were added, then the temperature was raised to 23° C., and the mixture was stirred for 16 hours. While the obtained reaction product was cooled to 10° C., 1.34 parts of a 5% oxalic acid aqueous solution were added and stirred for 30 minutes. 40 parts of ethyl acetate and 20 parts of ion-exchanged water were added to the obtained reaction product, and the mixture was stirred at 23° C. for 30 minutes, then separated, and an organic layer was taken out. This water washing operation was repeated five times. The organic layer thus obtained was concentrated and then the concentrated mixture was isolated using a column (silica gel 60N (spherical, neutral) 100-210 μm; manufactured by Kanto Chemical Co., Inc., developing solvent: ethyl acetate) to obtain 3.27 parts of a compound represented by formula (I-1151-e).

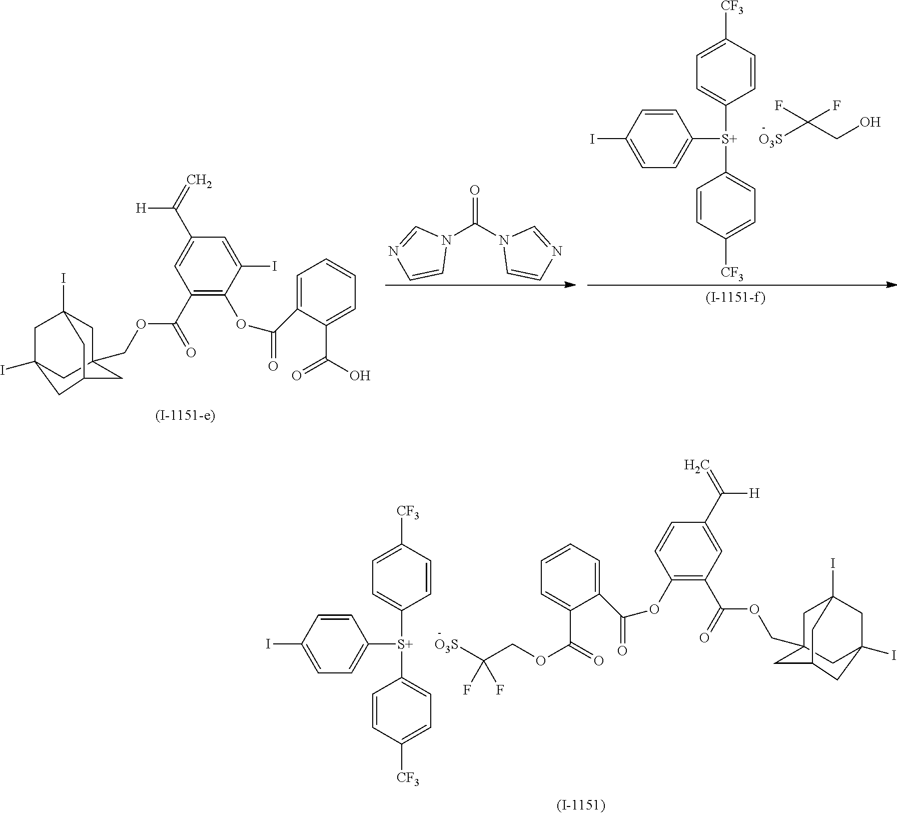

2.93 parts of a compound represented by a formula (J-1151-e) and 30 parts of chloroform were mixed, and the mixture was stirred at 23° C. for 30 minutes, then 0.57 parts of carbonyl diimidazole were added, and the temperature was raised to 60° C., then the mixture was stirred at 60° C. for 2 hours. To the reaction mixture thus obtained, 2.40 parts of a salt represented by a formula (I-1151-f) were added, followed by stirring at 60° C. for 3 hours and further cooling to 23° C. To the mixture thus obtained, 10 parts of an aqueous 5% oxalic acid solution were added and, after stirring at 23° C. for 30 minutes, the organic layer was taken out through separation. To the organic layer thus obtained, 10 parts of ion-exchanged water were added and, after stirring at 23° C. for 30 minutes, the organic layer was taken out through separation. This water washing operation was repeated five times. The obtained organic layer was concentrated, 30 parts of tert-butyl methyl ether were added to the concentrated residue, and the mixture was stirred, then the supernatant was removed, and the mixture was concentrated to obtain 3.96 parts of a salt represented by a formula (I-1151).

MS (ESI (+) Spectrum): M+525.0

MS (ESI (−) Spectrum): M−980.8

### Example 5: Synthesis of Salt Represented by Formula (I-1171)

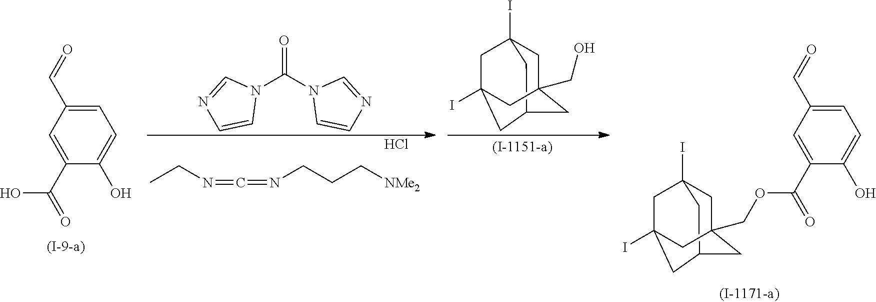

1.66 parts of a compound represented by a formula (1-9-a) and 20 parts of tetrahydrofuran were added, and the mixture was stirred at 23° C. for 30 minutes, and then charged with 1.62 parts of carbonyldiimidazole and 1.92 parts of 1-[3-(dimethylamino) propyl]-3-ethylcarbodiimide hydrochloride, and the mixture was stirred at 23° C. for 1 hour and then further stirred at 50° C. for 2 hours. 4.18 parts of a compound represented by a formula (I-1151-a) were added to the obtained reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm; development solvent: n-heptane/ethyl acetate=10/1, manufactured by KANTO CHEMICAL CO., INC.) to obtain 4.30 parts of a compound represented by a formula (1-1171-a).

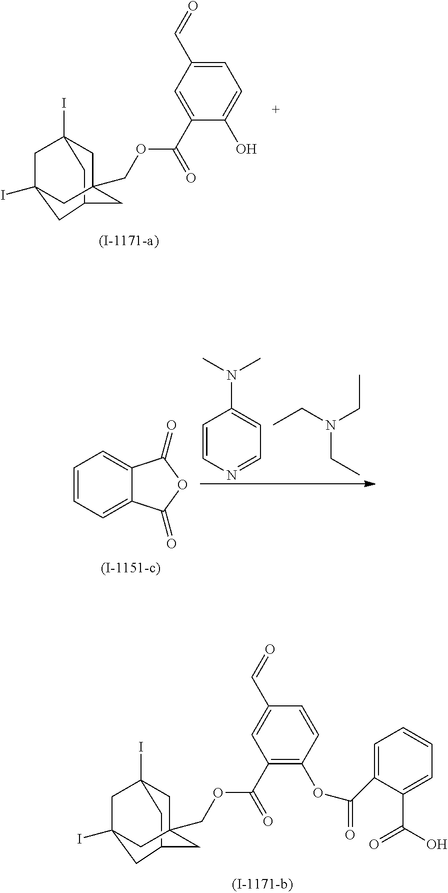

3.96 parts of a compound represented by a formula (I-1171-a), 0.94 parts of dimethylaminopyridine, 0.85 parts of triethylamine, and 40 parts of dimethylformamide were mixed, and the mixture was stirred at 23° C. for 30 minutes, then, 1.24 parts of a compound represented by a formula (I-1151-c) and 20 parts of chloroform were added thereto, and the mixture was stirred at 50° C. for 5 hours, and then further stirred at 23° C. for 2 hours. 10 parts of 1N hydrochloric acid were added to the obtained reaction mixture, and the mixture was stirred at 23° C. for 30 minutes and separated to obtain an organic layer. 30 parts of ion-exchanged water were added to the collected organic layer, and the mixture was stirred at 23° C. for 30 minutes and separated to obtain an organic layer. This water washing operation was performed three times. The organic layer thus obtained was concentrated and then the concentrated mixture was fractionated using a column (silica gel 60N (spherical, neutral) 100 to 210 μm, manufactured by KANTO CHEMICAL CO., INC., development solvent: ethyl acetate) to obtain 3.65 parts of a compound represented by a formula (I-1171-b).

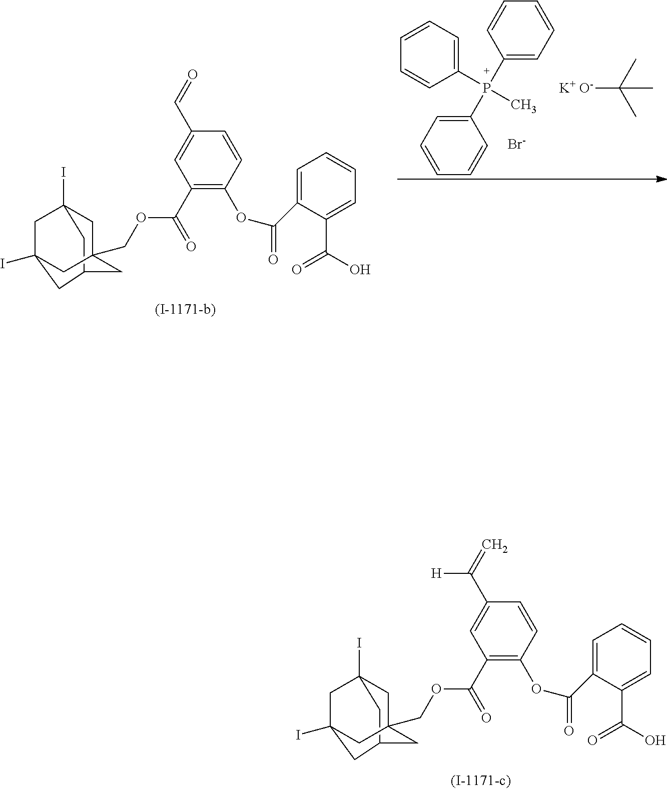

1.79 parts of methyltriphenylphosphonium bromide, 1.12 parts of potassium t-butoxide and 40 parts of tetrahydrofuran were mixed, and the mixture was stirred at 23° C. for 1 hour. While cooling the obtained reaction product to 10° C., 3.57 parts of a compound represented by a formula (1-1171-b) were added, then the temperature was raised to 23° C., and the mixture was stirred for 16 hours. While the obtained reaction product was cooled to 10° C., 1.34 parts of a 5% oxalic acid aqueous solution were added and stirred for 30 minutes. 40 parts of ethyl acetate and 20 parts of ion-exchanged water were added to the obtained reaction product, and the mixture was stirred at 23° C. for 30 minutes, then separated, and an organic layer was taken out. This water washing operation was repeated five times. The organic layer thus obtained was concentrated and then the concentrated mixture was fractionated using a column (silica gel 60N (spherical, neutral) 100 to 210 μm, manufactured by KANTO CHEMICAL CO., INC., development solvent: ethyl acetate) to obtain 2.85 parts of a compound represented by a formula (I-1171-c).

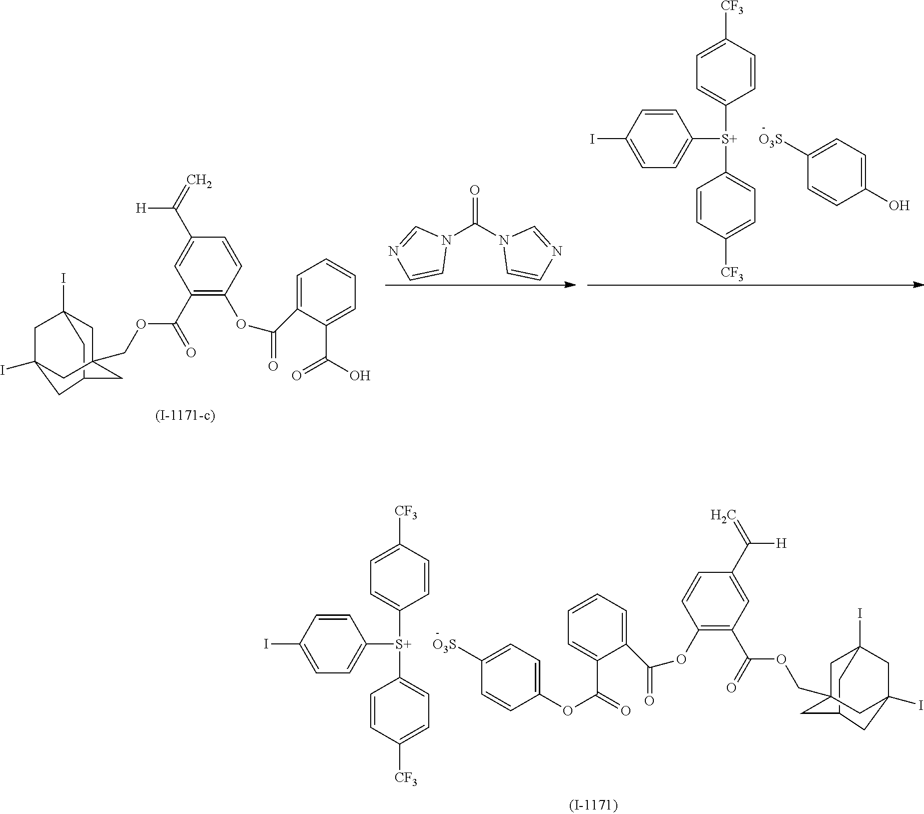

2.85 parts of a compound represented by a formula (1-1171-c) and 30 parts of chloroform were mixed, and the mixture was stirred at 23° C. for 30 minutes, then 0.65 parts of carbonyl diimidazole were added, and the temperature was raised to 60° C., then the mixture was stirred at 60° C. for 2 hours. To the reaction mixture thus obtained, 2.79 parts of a salt represented by a formula (I-1171-d) were added, followed by stirring at 60° C. for 3 hours and further cooling to 23° C. To the mixture thus obtained, 10 parts of an aqueous 5% oxalic acid solution were added and, after stirring at 23° C. for 30 minutes, the organic layer was taken out through separation. To the organic layer thus obtained, 10 parts of ion-exchanged water were added and, after stirring at 23° C. for 30 minutes, the organic layer was taken out through separation. This water washing operation was repeated five times. The obtained organic layer was concentrated, 30 parts of tert-butyl methyl ether were added to the concentrated residue, and the mixture was stirred, then the supernatant was removed, and the mixture was concentrated to obtain 4.23 parts of a salt represented by a formula (I-1171).

MS (ESI (+) Spectrum): M+525.0

MS (ESI (−) Spectrum): M−867.0

### Example 6: Synthesis of Salt Represented by Formula (I-1223)

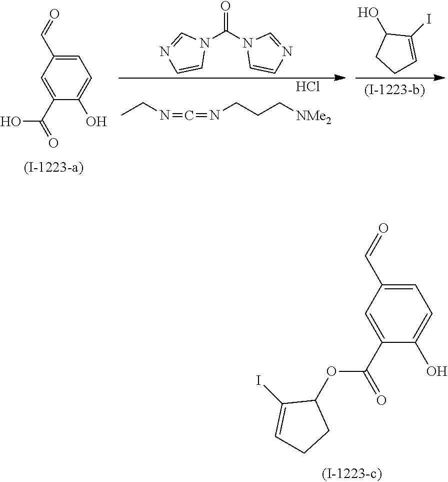

1.66 parts of a compound represented by a formula (I-1223-a) and 20 parts of tetrahydrofuran were added, and the mixture was stirred at 23° C. for 30 minutes, and then charged with 1.62 parts of carbonyldiimidazole and 1.92 parts of 1-[3- (dimethylamino) propyl]-3-ethylcarbodiimide hydrochloride, and the mixture was stirred at 23° C. for 1 hour and then further stirred at 50° C. for 2 hours. 2.10 parts of a compound represented by a formula (I-1223-b) were added to the obtained reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm; development solvent: n-heptane/ethyl acetate=3/1, manufactured by KANTO CHEMICAL CO., INC.) to obtain 2.54 parts of a compound represented by a formula (I-1223-c).

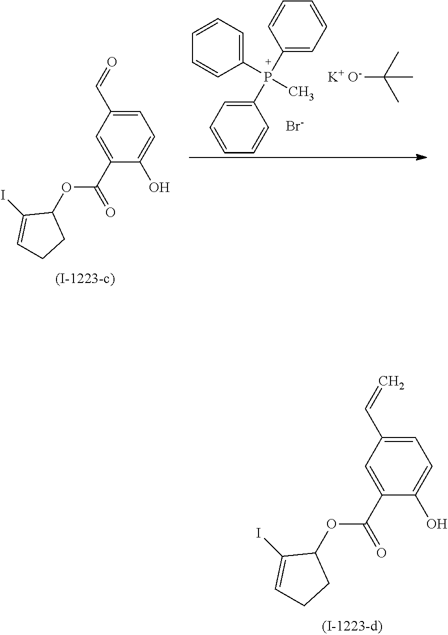

2.53 parts of methyltriphenylphosphonium bromide, 0.80 parts of potassium t-butoxide and 40 parts of tetrahydrofuran were mixed, and the mixture was stirred at 23° C. for 1 hour. While cooling the obtained reaction product to 10° C., 2.54 parts of a compound represented by a formula (I-1223-c) were added, then the temperature was raised to 23° C., and the mixture was stirred for 16 hours. While the obtained reaction product was cooled to 10° C., 2.24 parts of a 5% oxalic acid aqueous solution were added and stirred for 30 minutes. 40 parts of ethyl acetate and 20 parts of ion-exchanged water were added to the obtained reaction product, and the mixture was stirred at 23° C. for 30 minutes, then separated, and an organic layer was taken out. This water washing operation was repeated five times. The organic layer thus obtained was concentrated and then the concentrated mixture was fractionated using a column (silica gel 60N (spherical, neutral) 100 to 210 μm, manufactured by KANTO CHEMICAL CO. INC., development solvent: n-heptane/ethyl acetate=10/1) to obtain 1.95 parts of a compound represented by a formula (I-1223-d).

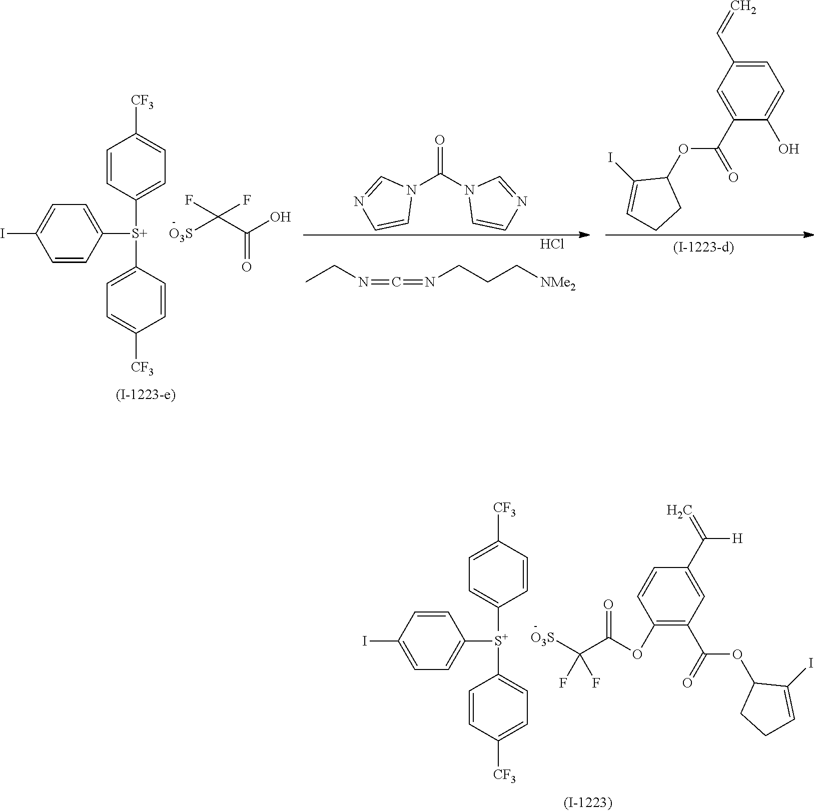

3.57 parts of a salt represented by a formula (I-1223-e) and 25 parts of acetonitrile were added, and the mixture was stirred at 23° C. for 30 minutes, and then charged with 0.89 parts of carbonyldiimidazole and 0.98 parts of 1-[3-(dimethylamino)propyl]-3-ethylcarbodiimide hydrochloride, and the mixture was stirred at 23° C. for 1 hour and then further stirred at 50° C. for 2 hours. 1.95 parts of a compound represented by a formula (I-1223-d) were added to the obtained reaction product, and the mixture was further stirred at 50° C. for 2 hours and then cooled to 23° C. 50 parts of chloroform and 30 parts of ion-exchanged water were added to the obtained reaction mass, and the mixture was stirred at 23° C. for 30 minutes, then separated, and an organic layer was taken out. This water washing operation was repeated four times. The organic layer thus obtained was concentrated and then 3 parts of acetonitrile and 30 parts of tert-butyl methyl ether were added to the concentrated residue and, after stirring at 23° C. for 30 minutes, the supernatant was removed, followed by concentration to obtain 3.87 parts of a salt represented by a formula (I-1223).

MS (ESI (+) Spectrum): M+525.0

MS (ESI (−) Spectrum): M−512.9

### Example 7: Synthesis of Salt Represented by Formula (I-1224)

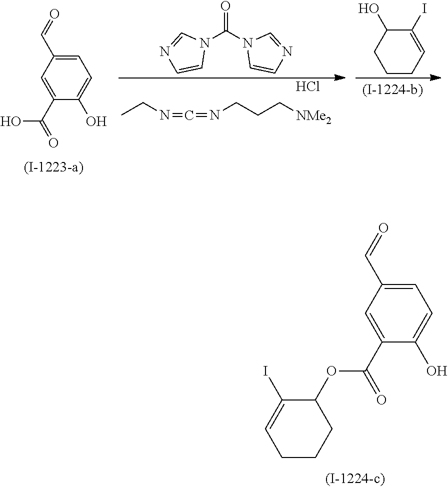

1.66 parts of a compound represented by a formula (I-1223-a) and 20 parts of tetrahydrofuran were added, and the mixture was stirred at 23° C. for 30 minutes, and then charged with 1.62 parts of carbonyldiimidazole and 1.92 parts of 1-[3- (dimethylamino) propyl]-3-ethylcarbodiimide hydrochloride, and the mixture was stirred at 23° C. for 1 hour and then further stirred at 50° C. for 2 hours. 2.24 parts of a compound represented by a formula (I-1224-b) were added to the obtained reaction product, and the mixture was further stirred at 50° C. for 2 hours and then cooled to 23° C. 50 parts of ethyl acetate and 30 parts of ion-exchanged water were added to the obtained reaction mass, and the mixture was stirred at 23° C. for 30 minutes, then separated, and an organic layer was taken out. This water washing operation was repeated four times. The obtained organic layer was concentrated, and the concentrated mass was fractionated by a column (silica gel 60 N (spherical, neutral) 100 to 210 μm; development solvent: n-heptane/ethyl acetate=4/1, manufactured by KANTO CHEMICAL CO., INC.) to obtain 2.90 parts of a compound represented by a formula (I-1224-c).

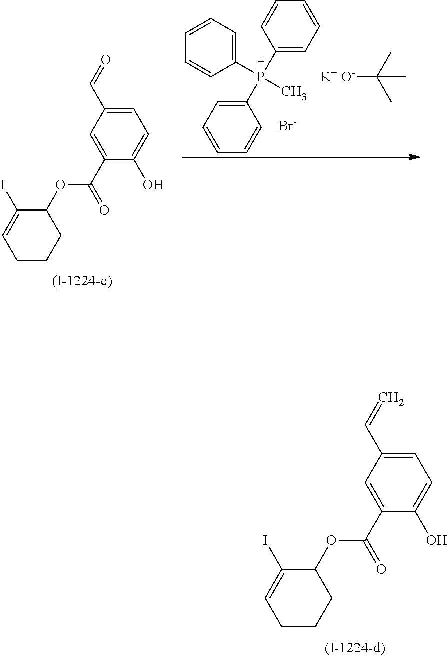

2.79 parts of methyltriphenylphosphonium bromide, 0.88 parts of potassium t-butoxide, and 40 parts of tetrahydrofuran were mixed, and the mixture was stirred at 23° C. for 1 hour. While cooling the obtained reaction product to 10° C., 2.90 parts of a compound represented by a formula (I-1224-c) were added, then the temperature was raised to 23° C., and the mixture was stirred for 16 hours. While the obtained reaction product was cooled to 10° C., 2.24 parts of a 5% oxalic acid aqueous solution were added and stirred for 30 minutes. 40 parts of ethyl acetate and 20 parts of ion-exchanged water were added to the obtained reaction product, and the mixture was stirred at 23° C. for 30 minutes, then separated, and an organic layer was taken out. This water washing operation was repeated five times. The organic layer thus obtained was concentrated and then the concentrated mixture was fractionated using a column (silica gel 60N (spherical, neutral) 100 to 210 μm, manufactured by KANTO CHEMICAL CO. INC., development solvent: n-heptane/ethyl acetate=10/1) to obtain 2.34 parts of a compound represented by a formula (I-1224-d).

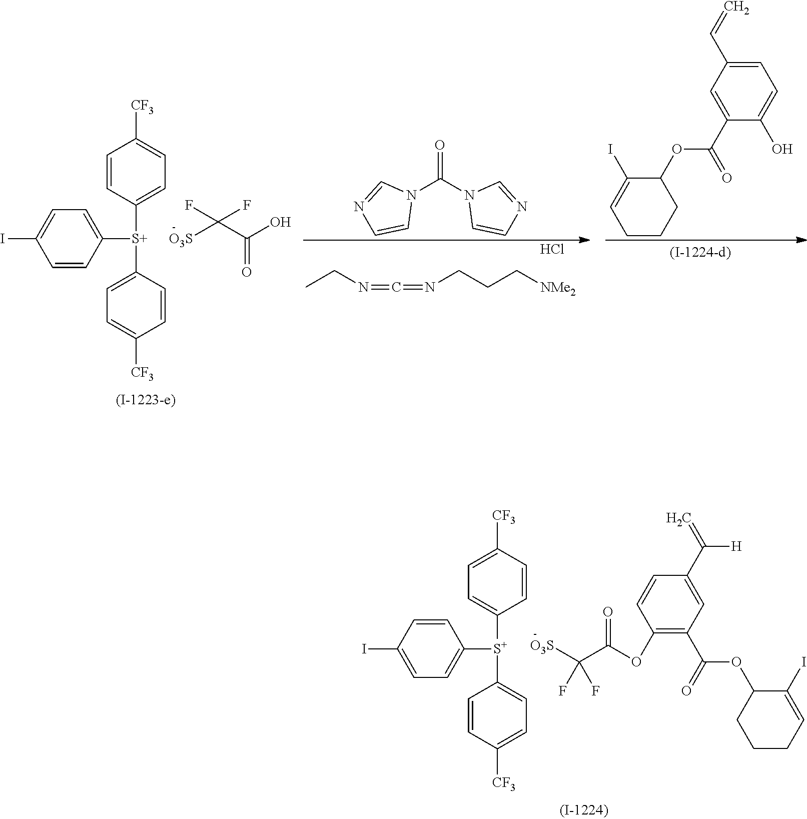

4.41 parts of a salt represented by a formula (I-1223-e) and 25 parts of acetonitrile were added, and the mixture was stirred at 23° C. for 30 minutes, and then charged with 1.02 parts of carbonyldiimidazole and 1.21 parts of 1-[3-(dimethylamino)propyl]-3-ethylcarbodiimide hydrochloride, and the mixture was stirred at 23° C. for 1 hour and then further stirred at 50° C. for 2 hours. 2.34 parts of a compound represented by a formula (I-1224-d) were added to the obtained reaction product, and the mixture was further stirred at 50° C. for 2 hours and then cooled to 23° C. 50 parts of chloroform and 30 parts of ion-exchanged water were added to the obtained reaction mass, and the mixture was stirred at 23° C. for 30 minutes, then separated, and an organic layer was taken out. This water washing operation was repeated four times. The organic layer thus obtained was concentrated and then 3 parts of acetonitrile and 30 parts of tert-butyl methyl ether were added to the concentrated residue and, after stirring at 23° C. for 30 minutes, the supernatant was removed, followed by concentration to obtain 5.24 parts of a salt represented by a formula (I-1224).

MS (ESI (+) Spectrum): M+525.0

MS (ESI (−) Spectrum): M−527.0

### Synthesis of Resin

Compounds (monomers) used in synthesis of a resin are shown below.

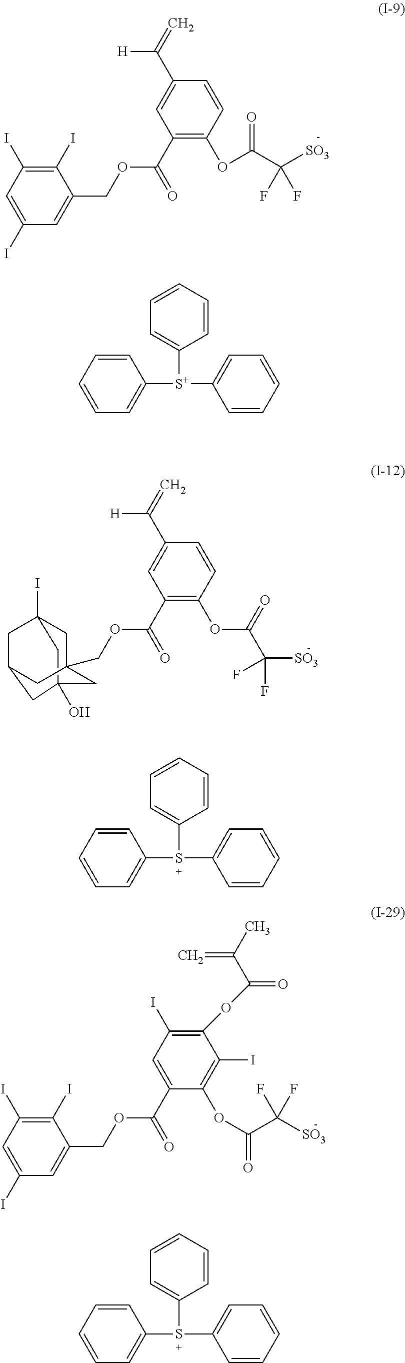

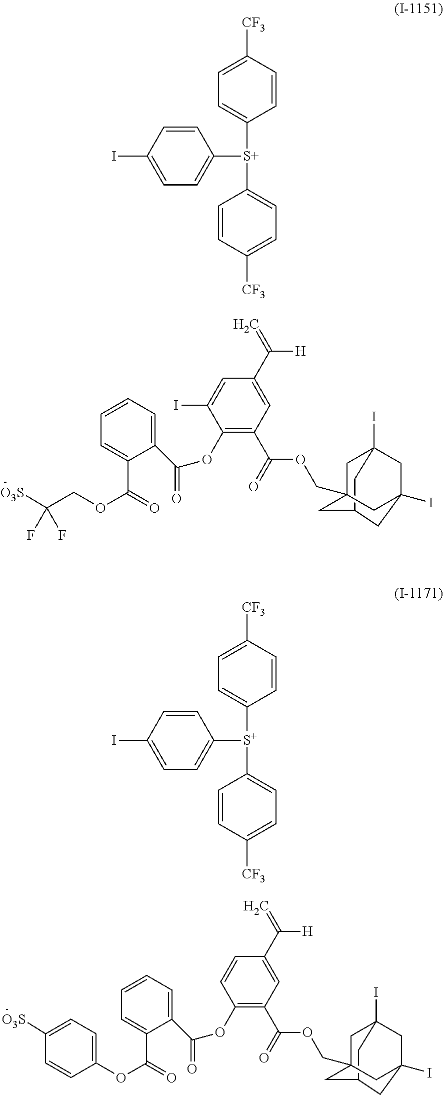

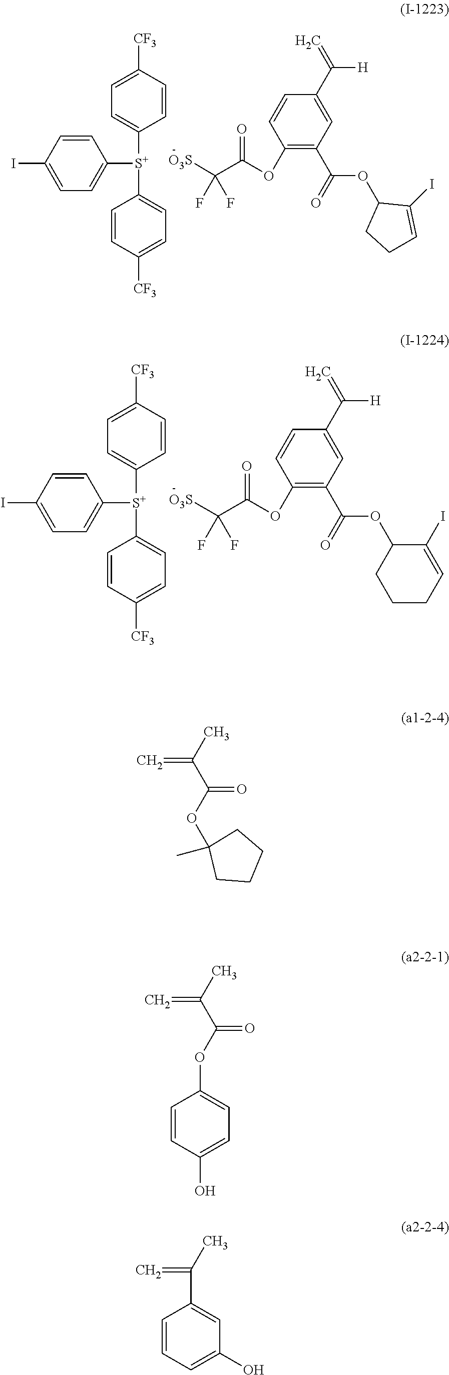

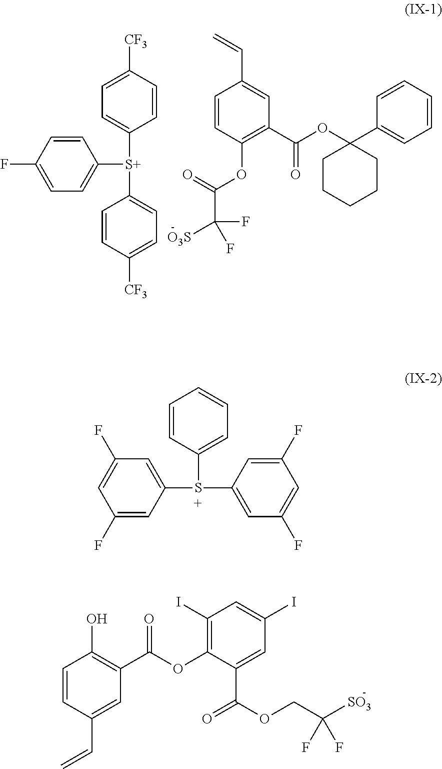

Hereinafter, these compounds are referred to as “monomer (a2-2-1)” or the like according to the formula number.

### Example 8 [Synthesis of Resin A¹]

Using a monomer (a2-2-1), a monomer (a1-2-4), and a monomer (I-9) as monomers, these monomers were mixed in a molar ratio of 38:47:15 [monomer (a2-2-1):monomer (a1-1-4):monomer (I-9)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A1 (copolymer) having a weight-average molecular weight of about 6.4×10³ in a yield of 88%. This resin A1 has the following structural units.

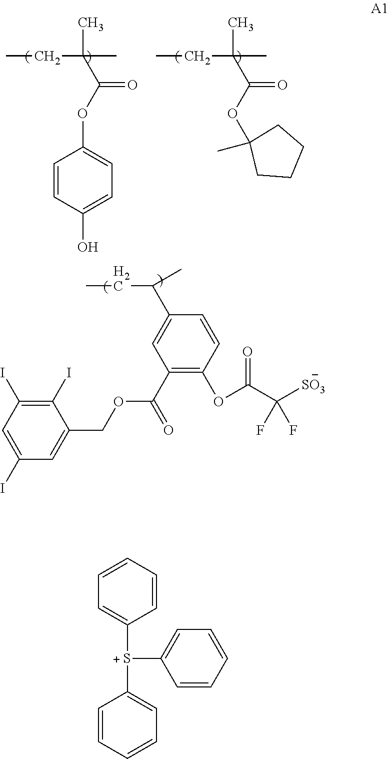

### Example 9 [Synthesis of Resin A2]

Using a monomer (a2-2-1), a monomer (a1-2-4), and a monomer (I-12) as monomers, these monomers were mixed in a molar ratio of 38:47:15 [monomer (a2-2-1):monomer (a1-2-4):monomer (I-12)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A2 (copolymer) having a weight-average molecular weight of about 6.0×10³ in a yield of 83%. This resin A2 has the following structural units.

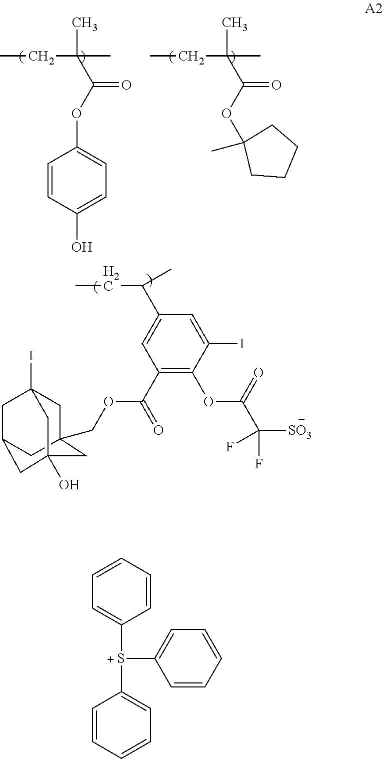

### Example 10 [Synthesis of Resin A3]

Using a monomer (a2-2-1), a monomer (a1-2-4), and a monomer (I-29) as monomers, these monomers were mixed in a molar ratio of 38:47:15 [monomer (a2-2-1):monomer (a1-2-4):monomer (I-29)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A3 (copolymer) having a weight-average molecular weight of about 6.8×10³ in a yield of 90%. This resin A3 has the following structural units.

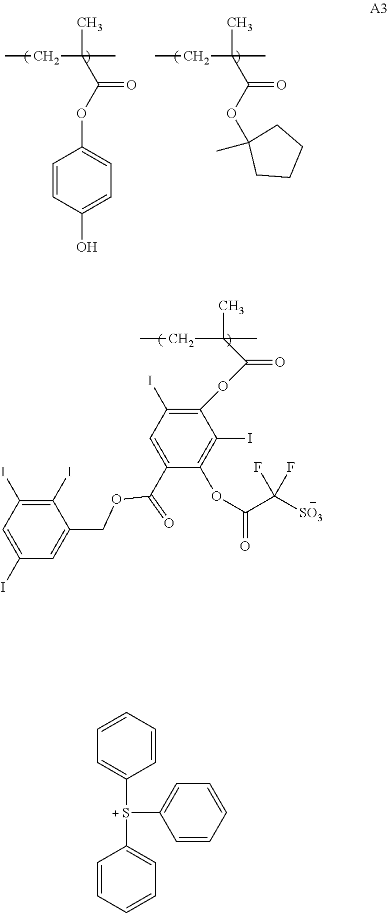

### Example 11 [Synthesis of Resin A4]

Using a monomer (a2-2-1), a monomer (a1-2-4), and a monomer (I-1151) as monomers, these monomers were mixed in a molar ratio of 38:47:15 [monomer (a2-2-1):monomer (a1-2-4):monomer (I-1151)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A4 (copolymer) having a weight-average molecular weight of about 6.0×10³ in a yield of 80%. This resin A4 has the following structural units.

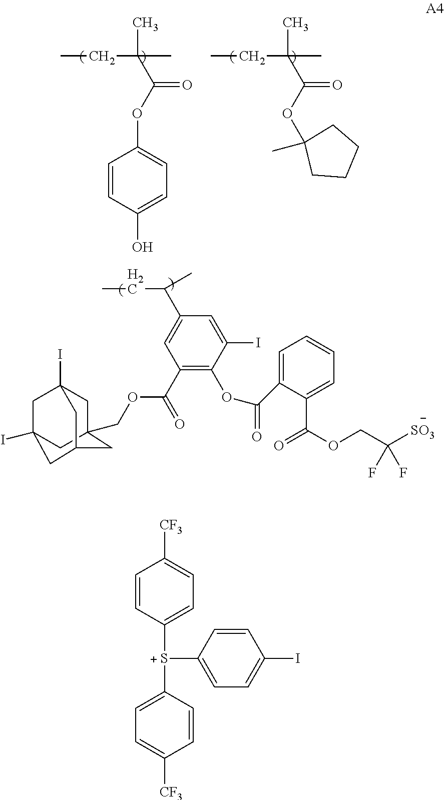

### Example 12 [Synthesis of Resin A5]

Using the monomer (a2-2-1), the monomer (a1-2-4), and a monomer (I-1171) as monomers, these monomers were mixed in a molar ratio of 38:47:15 [monomer (a2-2-1):monomer (a1-2-4):monomer (I-1171)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A5 (copolymer) having a weight-average molecular weight of about 5.9×10³ in a yield of 85%. This resin A5 has the following structural units.

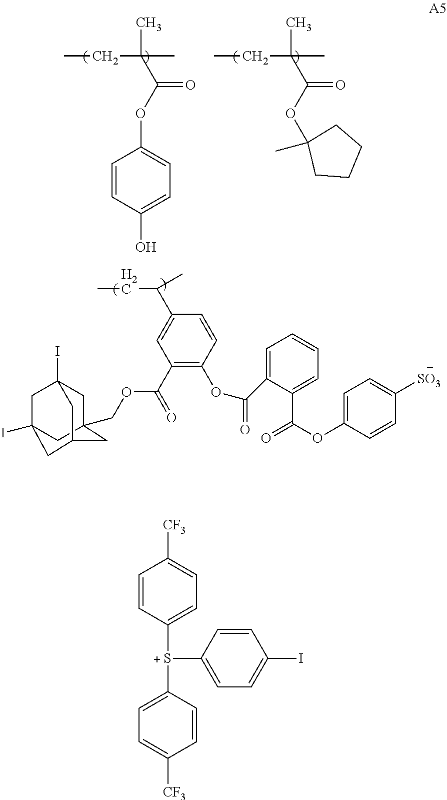

### Example 13 [Synthesis of Resin A6]

Using the monomer (a2-2-4), the monomer (a1-2-4), and the monomer (I-9) as monomers, these monomers were mixed in a molar ratio of 38:47:15 [monomer (a2-2-4):monomer (a1-2-4):monomer (I-9)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A6 (copolymer) having a weight-average molecular weight of about 5.7×10³ in a yield of 81%. This resin A6 has the following structural units.

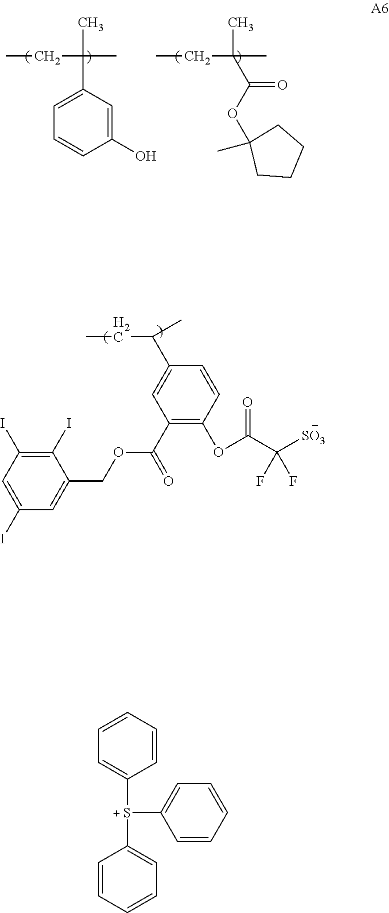

### Example 14 [Synthesis of Resin A7]

Using the monomer (a2-2-1), the monomer (a1-2-4), and a monomer (I-1223) as monomers, these monomers were mixed in a molar ratio of 38:47:15 [monomer (a2-2-1):monomer (a1-2-4):monomer (I-1223)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A7 (copolymer) having a weight-average molecular weight of about 6.0×10³ in a yield of 83%. This resin A7 has the following structural units.

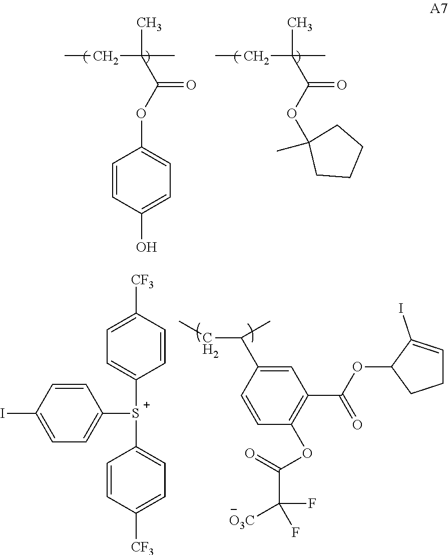

### Example 15 [Synthesis of Resin A8]

Using the monomer (a2-2-1), the monomer (a1-2-4), and a monomer (I-1224) as monomers, these monomers were mixed in a molar ratio of 38:47:15 [monomer (a2-2-1):monomer (a1-2-4):monomer (I-1224)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin A8 (copolymer) having a weight-average molecular weight of about 6.1×10³ in a yield of 85%. This resin A8 has the following structural units.

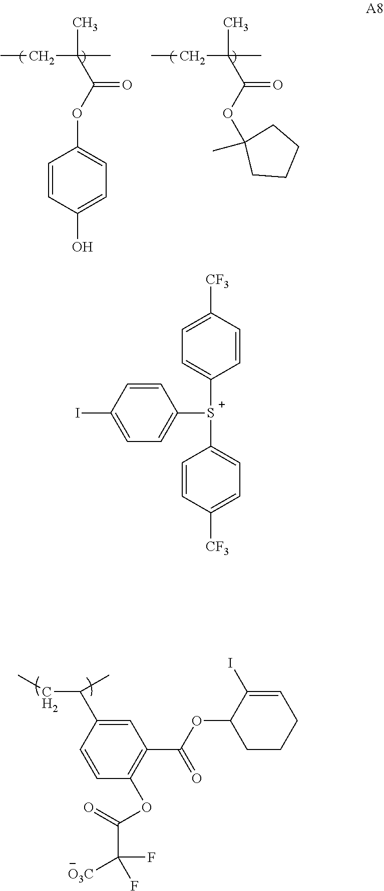

### Synthesis Example 1 [Synthesis of Resin AX1]

Using the monomer (a2-2-1), the monomer (a1-2-4), and a monomer (IX-1) as monomers, these monomers were mixed in a molar ratio of 38:47:15 [monomer (a2-2-1):monomer (a1-2-4):monomer (IX-1)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin AX1 (copolymer) having a weight-average molecular weight of about 5.4×10³ in a yield of 83%. This resin AX1 has the following structural units.

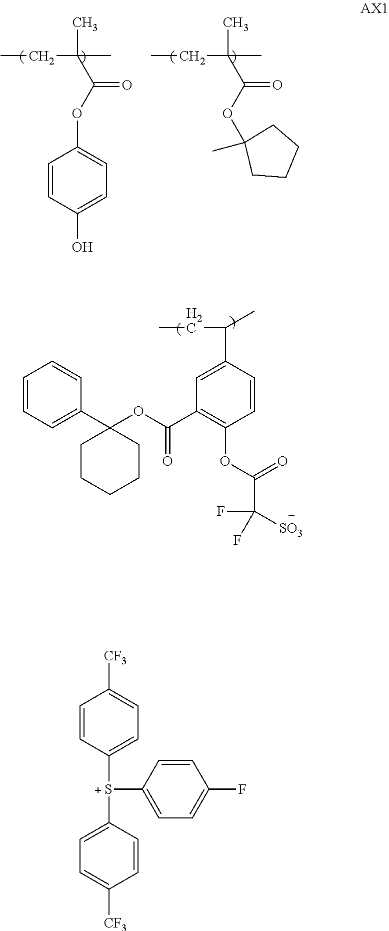

### Synthesis Example 2 [Synthesis of Resin AX2]

Using the monomer (a2-2-1), the monomer (a1-2-4), and a monomer (IX-2) as monomers, these monomers were mixed in a molar ratio of 38:47:15 [monomer (a2-2-1):monomer (a1-2-4):monomer (IX-2)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 85° C. for about 5 hours. Thereafter, the polymerization reaction solution thus obtained was poured into a large amount of n-heptane to precipitate a resin, followed by filtration and recovery to obtain a resin AX2 (copolymer) having a weight-average molecular weight of about 5.9×10³ in a yield of 87%. This resin AX2 has the following structural units.

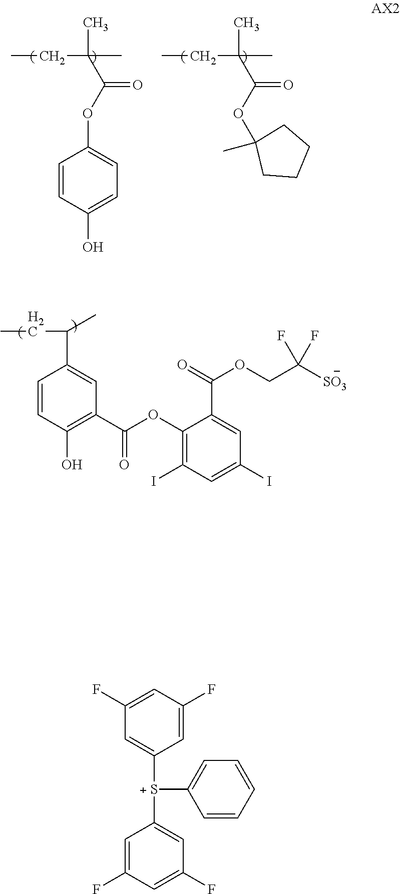

### <Preparation of Resist Composition>

The respective components shown in Table 2 and the following solvents were mixed and the mixture thus obtained was filtered through a fluororesin filter having a pore size of 0.2 μm to prepare resist compositions.

TABLE 2

| Resist | | Acid | | | |
|---|---|---|---|---|---|
| composi- | Resin | generator | Salt | Quencher | |
| tion | (A) | (B) | (I) | (C) | PB/PEB |
| Composi- | A1 = 10 | — | — | C1-1 = 0.35 | 100° C./ |
| tion 1 | parts | | | parts | 130° C. |
| Composi- | A2 = 10 | — | — | C1-1 = 0.35 | 100° C./ |
| tion 2 | parts | | | parts | 130° C. |
| Composi- | A3 = 10 | — | — | C1-1 = 0.35 | 100° C./ |
| tion 3 | parts | | | parts | 130° C. |
| Composi- | A4 = 10 | — | — | C1-1 = 0.35 | 100° C./ |
| tion 4 | parts | | | parts | 130° C. |
| Composi- | A5 = 10 | — | — | C1-1 = 0.35 | 100° C./ |
| tion 5 | parts | | | parts | 130° C. |
| Composi- | A6 = 10 | — | — | C1-1 = 0.35 | 100° C./ |
| tion 6 | parts | | | parts | 130° C. |
| Composi- | A1 = 10 | — | I-9 = | C1-1 = 0.35 | 100° C./ |
| tion 7 | parts | | 0.3 parts | parts | 130° C. |
| Composi- | A1 = 10 | B1-25 = | — | C1-1 = 0.35 | 100° C./ |
| tion 8 | parts | 0.3 parts | | parts | 130° C. |
| Composi- | A7 = 10 | — | — | C1-1 = 0.35 | 100° C./ |
| tion 9 | parts | | | parts | 130° C. |
| Composi- | A8 = 10 | — | — | C1-1 = 0.35 | 100° C./ |
| tion 10 | parts | | | parts | 130° C. |
| Compar- | AX1 = 10 | — | — | C1-1 = 0.35 | 100° C./ |
| ative | parts | | | parts | 130° C. |
| Composi- | | | | | |
| tion 1 | | | | | |
| Compar- | AX2 = 10 | — | — | C1-1 = 0.35 | 100° C./ |
| ative | parts | | | parts | 130° C. |
| Composi- | | | | | |
| tion 2 | | | | | |

### <Resin (A)>

A1 to A8, AX1, and AX2: resin A1 to resin A8, resin AX1, and resin AX2

### <Salt (I)>

I-9: Salt represented by formula (I-9)

### <Acid Generator (B)>

B1-25: Salt represented by formula (B1-25), synthesized by the method described in JP-A-2011-126869

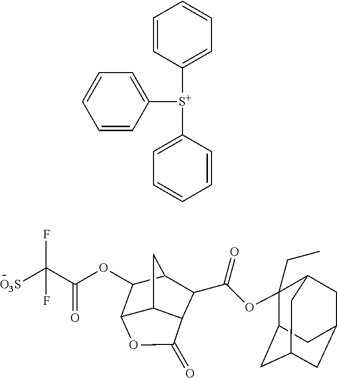

### <Quencher (C)>

C1-1: synthesized by the method described in JP-A-2011-39502

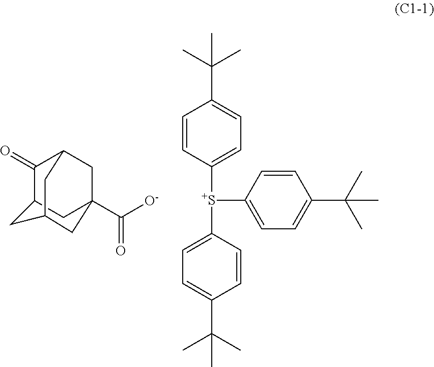 [SMILES: O=C1C2CC3CC1CC(C(=O)[O-])(C3)C2 ; CC(C)(C)c1ccc([S+](c2ccc(C(C)(C)C)cc2)c2ccc(C(C)(C)C)cc2)cc1]

### <Solvent (E)>

| | |
|---|---|
| Propylene glycol monomethyl ether acetate | 400 parts |
| Propylene glycol monomethyl ether | 100 parts |
| γ-Butyrolactone | 5 parts |

### (Evaluation of Electron Beam Exposure of Resist Composition: Organic Solvent Development)

Each 6 inch-diameter silicon wafer was treated with hexamethyldisilazane on a direct hot plate at 90° C. for 60 seconds. A resist composition was spin-coated on the silicon wafer in such a manner that the thickness of the composition layer became 0.04 μm. Then, the coated silicon wafer was prebaked on the direct hot plate at the temperature shown in the column “PB” of Table 2 for 60 seconds to form a composition layer. Using an electron-beam direct-write system (“ELS-F125 125 keV”, manufactured by ELIONIX INC.), contact hole patterns (hole pitch of 40 nm/hole diameter of 17 nm) were directly written on the composition layer formed on the wafer while changing the exposure dose stepwise.

After exposure, post-exposure baking was performed on the hot plate at the temperature shown in the column “PEB” of Table 2 for 60 seconds. Then, the composition layer on the silicon wafer was developed by a dynamic dispensing method at 23° C. for 20 seconds using butyl acetate (manufactured by Tokyo Chemical Industry Co., Ltd.) as a developer to obtain a resist pattern.

In the resist pattern obtained after development, the exposure dose at which the diameter of holes formed became 17 nm was defined as effective sensitivity.

### <Evaluation of CD Uniformity (CDU)>

In the effective sensitivity, the hole diameter of the pattern formed with a hole diameter of 17 nm was determined by measuring 24 times per one hole and the average of the measured values was regarded as the average hole diameter per one hole. The standard deviation was determined under the conditions that the values obtained by measuring the average diameter of 400 holes about the patterns formed with a hole diameter of 17 nm in the same wafer was regarded as the population.

The results are shown in Table 3. The numerical value in the table represents the standard deviation (nm).

TABLE 3

| | Resist | |
|---|---|---|
| | composition | CDU |
| Example 16 | Composition 1 | 2.15 |
| Example 17 | Composition 2 | 2.24 |
| Example 18 | Composition 3 | 2.33 |
| Example 19 | Composition 4 | 2.09 |
| Example 20 | Composition 5 | 2.27 |
| Example 21 | Composition 6 | 2.13 |
| Example 22 | Composition 7 | 2.19 |
| Example 23 | Composition 8 | 2.31 |
| Example 24 | Composition 9 | 2.07 |
| Example 25 | Composition 10 | 2.06 |
| Comparative | Comparative | 2.52 |
| Example 1 | Composition 1 | |
| Comparative | Comparative | 2.43 |
| Example 2 | Composition 2 | |

As compared with Comparative Compositions 1 and 2, Compositions 1 to 10 exhibited small standard deviation and satisfactory evaluation of CD uniformity (CDU).

A resist composition of the present invention exhibits satisfactory CD uniformity (CDU), and is therefore useful for fine processing of semiconductors.

What is claimed is:

1. An acid generator comprising a salt represented by a formula (I) or a structural unit represented by a formula (IP), wherein 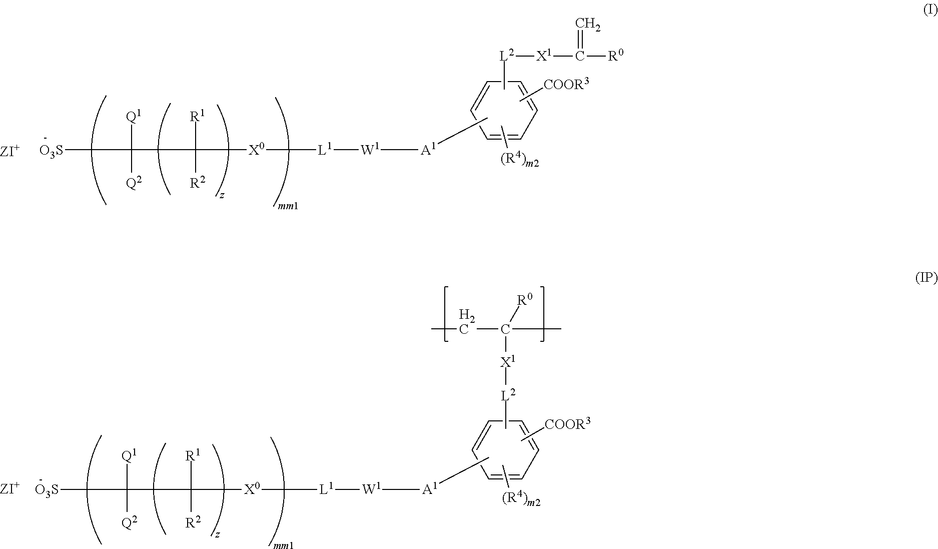
Q¹, Q², R¹, and R² each independently represent a hydrogen atom, a fluorine atom, a perfluoroalkyl group having 1 to 6 carbon atoms, or an alkyl group having 1 to 6 carbon atoms,
z represents an integer of 0 to 6, and when z is 2 or more, a plurality of R¹ and R² may be same or different from each other,
X⁰ represents a single bond, or a group formed by —O—, —CO—, —S—, —SO—, —SO₂—, —NR⁷—, or a combination thereof,
mm1 represents 0 or 1,
L¹ and L² each independently represent a single bond or a hydrocarbon group having 1 to 28 carbon atoms and optionally having a substituent, and —CH₂— included in the hydrocarbon group may be substituted with —O—, —S—, —SO₂—, —SO—, —NR⁷—, or —CO—,
R⁷ represents a hydrogen atom or an alkyl group having 1 to 6 carbon atoms,
W¹ represents a single bond or a cyclic hydrocarbon group having 3 to 36 carbon atoms and optionally having a substituent, and —CH₂— included in the cyclic hydrocarbon group may be substituted with —O—, —S—, —SO₂—, —SO—, —NR⁷—, or —CO—,
A¹ represents a single bond or a hydrocarbon group having 1 to 20 carbon atoms, the hydrocarbon group optionally has a substituent, and —CH₂— included in the hydrocarbon group may be substituted with —O—, —S—, —SO₂—, —SO—, —NR⁷—, or —CO—,
R³ represents an acid stable hydrocarbon group optionally having a substituent,
R⁴ represents a halogen atom, a cyano group, a nitro group, or a hydrocarbon group having 1 to 18 carbon atoms, the hydrocarbon group optionally has a substituent, and —CH₂— included in the hydrocarbon group may be substituted with —O—, —S—, —SO₂—, —SO—, —NR⁷—, or —CO—,
m2 represents an integer of 0 to 3, and when m2 is 2 or more, a plurality of R⁴ may be same or different from each other,
X¹ represents a single bond, or —O—, —CO—, —S—, —SO—, —SO₂—, —NR⁷—, a phenylene group optionally having a substituent, or a group obtained by combining these groups,
R⁰ represents a hydrogen atom, a halogen atom, or an alkyl group having 1 to 6 carbon atoms and optionally having a halogen atom, and
ZI⁺ represents an organic cation.

2. The acid generator according to claim 1, wherein
A¹ is a single bond, ***-X⁰¹-L⁰¹-, or ***-L⁰¹-X⁰¹-,
X⁰¹ represents —O—, —CO—, —S—, —SO—, —NR⁷—, or —SO₂—,
L⁰¹ represents a single bond or a hydrocarbon group having 1 to 18 carbon atoms wherein the hydrocarbon group may have a substituent, and —CH₂— included in the hydrocarbon group may be substituted with —O—, —CO—, —S—, —SO—, —NR⁷—, or —SO₂—, and
*** represents a binding site to W¹.

3. The acid generator according to claim 2, wherein
X⁰¹ is —O— or —CO—, and
L⁰¹ is a single bond or an alkanediyl group having 1 to 6 carbon atoms wherein —CH₂— included in the alkanediyl group may be substituted with —O— or —CO—.

4. The acid generator according to claim 1, wherein W¹ is a single bond or an aromatic hydrocarbon group having 6 to 10 carbon atoms and optionally having a substituent.

5. The acid generator according to claim 1, wherein
an acid stable hydrocarbon group optionally having a substituent of R³ is a group represented by a formula (10a-1) or a group represented by a formula (10a-2), 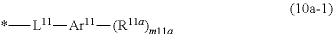
in the formula (10a-1),
L¹¹ represents a single bond or an alkanediyl group having 1 to 6 carbon atoms,
Ar¹¹ represents an aromatic hydrocarbon group having 6 to 18 carbon atoms,
R^{11a} represents a halogen atom or an alkyl group having 1 to 12 carbon atoms and optionally having a halogen atom, and —CH₂— included in the alkyl group may be substituted with —O— or —CO—,
m11a represents an integer of 0 to 8, when m11a is 2 or more, a plurality of R^{11a} may be same or different from each other, and
* represents a binding site, and 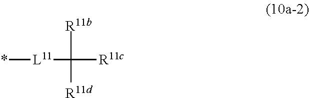
in the formula (10a-2),
L¹¹ represents a single bond or an alkanediyl group having 1 to 6 carbon atoms, for R^{11b}, R^{11c}, and R^{11d},
R^{11b} and R^{11c} each independently represent a hydrogen atom or a hydrocarbon group having 1 to 18 carbon atoms, and R^{11d} is a hydrogen atom, or
R^{11b} and R^{11c} are bonded to each other to form an alicyclic hydrocarbon group having 3 to 36 carbon atoms and optionally having a halogen atom or optionally having an alkyl group having 1 to 12 carbon atoms and optionally having a halogen atom wherein —CH₂— included in the alkyl group may be substituted with —O— or —CO—, and R^{11d} is a hydrogen atom or
R^{11b}, R^{11c}, and R^{11d} are bonded to each other to form an alicyclic hydrocarbon group having 3 to 36 carbon atoms and optionally having a halogen atom or optionally having an alkyl group having 1 to 12 carbon atoms and optionally having a halogen atom wherein —CH₂— included in the alkyl group may be substituted with —O— or —CO—, and
* represents a binding site.

6. The acid generator according to claim 1, wherein an acid stable hydrocarbon group optionally having a substituent of R³ is a group including a cyclic hydrocarbon group having 3 to 18 carbon atoms and having at least one selected from the group consisting of a halogen atom and a haloalkyl group having 1 to 12 carbon atoms wherein the cyclic hydrocarbon group may have a substituent other than a halogen atom and a haloalkyl group.

7. The acid generator according to claim 1, wherein m2 is an integer of 1 or more, and R⁴ is at least one selected from the group consisting of a halogen atom and a hydrocarbon group having 1 to 18 carbon atoms wherein —CH₂— included in the hydrocarbon group may be substituted with —O— or —CO—.

8. The acid generator according to claim 1, wherein X¹ is a single bond or a group represented by any one of a formula (X¹-1) to a formula (X¹-10), 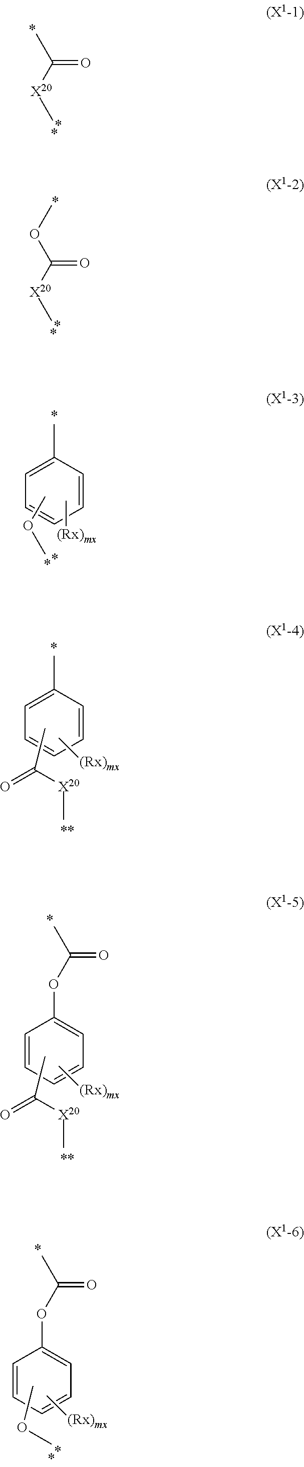 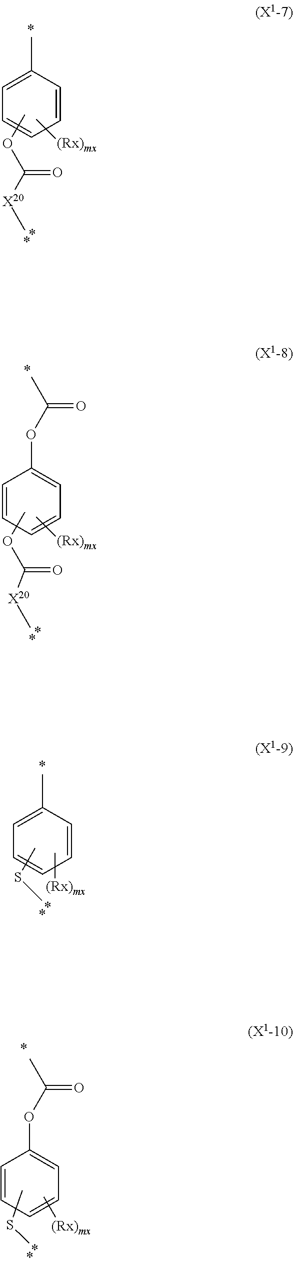
in the formula (X¹-1) to the formula (X¹-10),
* and ** are binding sites, and * represents a binding site to carbon atoms to which R⁰ is bonded,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms,
mx represents an integer of 0 to 4, When mx is 2 or more, a plurality of Rx may be the same or different from each other.
X²⁰ represents —O— or —NR⁷—, and
R⁷ represents a hydrogen atom or an alkyl group having 1 to 6 carbon atoms.

9. A resist composition comprising the acid generator according to claim 1.

10. The resist composition according to claim 9, wherein
an acid generator is a salt represented by a formula (I), and
the resist composition further comprises a resin including a structural unit (a1) having an acid-labile group.

11. The resist composition according to claim 9, wherein
an acid generator is a resin including a structural unit represented by a formula (IP), and
the resin further includes the structural unit (a1) having an acid-labile group.

12. The resist composition according to claim 11, further comprising a salt represented by the formula (I).

13. The resist composition according to claim 10, wherein
the structural unit (a1) having an acid-labile group includes at least one selected from the group consisting of a structural unit represented by a formula (a1-0), a structural unit represented by a formula (a1-1), a structural unit represented by a formula (a1-2), a structural unit represented by a formula (a1-4), a structural unit represented by a formula (a1-5), and a structural unit represented by a formula (a1-6), 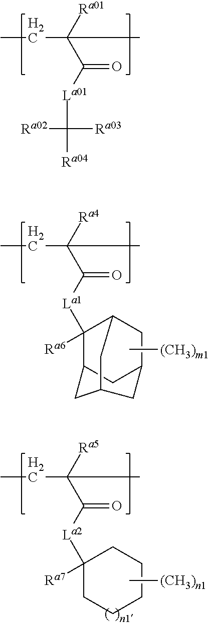
in the formula (a1-0), the formula (a1-1), and the formula (a1-2),
L^{a01}, L^{a1}, and L^{a2} each independently represent —O— or *—O—(CH₂)ₖ₁—CO—O—, k1 represents an integer of 1 to 7, and * represents a binding site to —CO—,
R^{a01}, R^{a4}, and R^{a5} each independently represent a hydrogen atom, a halogen atom, or an alkyl group having 1 to 6 carbon atoms and optionally having a halogen atom,
R^{a02}, R^{a03}, and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group, and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alkenyl group, the alicyclic hydrocarbon group, and the aromatic hydrocarbon group may have a halogen atom,
m1′ represents an integer of 0 to 14,
n1 represents an integer of 0 to 14, and
n1′ represents an integer of 0 to 3, and 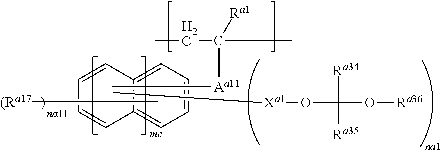
in the formula (a1-4),
R^{a1} represents a hydrogen atom, a halogen atom, or an alkyl group having 1 to 6 carbon atoms and optionally having a halogen atom,
R^{a17} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group,
A^{a11} represents a single bond or an alkanediyl group having 1 to 12 carbon atoms, and —CH₂— included in the alkanediyl group may be substituted with —O—, —CO— or —NR^{a18}—,
R^{a18} represents a hydrogen atom or an alkyl group having 1 to 6 carbon atoms,
X^{a1} represents a single bond or a carbonyl group,
R^{a34} and R^{a35} each independently represent a hydrogen atom or a hydrocarbon group having 1 to 12 carbon atoms, R^{a36} represents a hydrocarbon group having 1 to 20 carbon atoms, or R^{a34} represents a hydrogen atom or a hydrocarbon group having 1 to 12 carbon atoms, R^{a35} and R^{a36} may be bonded to each other to form a divalent heterocyclic group having 3 to 20 carbon atoms together with —C—O— to which they are bonded, and —CH₂— included in the hydrocarbon group and the divalent heterocyclic group may be substituted with —O— or —S—,
na1 represents an integer of 1 to 5, and when na1 is 2 or more, a plurality of groups in parentheses may be same or different from each other,
na11 represents an integer of 0 to 4, and when na11 is 2 or more, a plurality of R^{a17} may be same or different from each other, and
mc represents an integer of 0 to 2, and 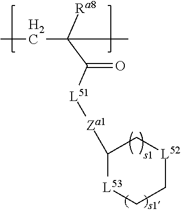
in the formula (a1-5),
R^{a8} represents an alkyl group having 1 to 6 carbon atoms and optionally having a halogen atom, a hydrogen atom, or a halogen atom,
Z^{a1} represents a single bond or *—(CH₂)ₕ₃—CO-L⁵⁴-, h3 represents an integer of 1 to 4, and * represents a binding site to L⁵¹,
L⁵¹, L⁵², L⁵³, and L⁵⁴ each independently represent —O— or —S—,
s1 represents an integer of 1 to 3, and
s1′ represents an integer of 0 to 3, and 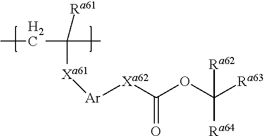
in the formula (a1-6),
R^{a61} represents a hydrogen atom, a halogen atom, or an alkyl group having 1 to 6 carbon atoms and optionally having a halogen atom,
R^{a62}, R^{a63}, and R^{a64} each independently represent an alkyl group having 1 to 6 carbon atoms or a cyclic hydrocarbon group having 3 to 18 carbon atoms and optionally having a substituent, or R^{a62} and R^{a63} are bonded to each other to form a ring having 3 to 20 carbon atoms together with the carbon atom to which they are bonded, and R^{a64} represents an alkyl group having 1 to 6 carbon atoms or a cyclic hydrocarbon group having 3 to 18 carbon atoms and optionally having a substituent,
X^{a61} represents a single bond, —CO—O—*, or —CO—NR^{a65}—*, * represents a binding site to Ar, and R^{a65} represents a hydrogen atom or an alkyl group having 1 to 6 carbon atoms,
X^{a62} represents a single bond, *—O-L^{a61}- or *—CO—O-L^{a62}-, * represents a binding site to Ar, and La⁶¹ and La⁶² each independently represent an alkanediyl group having 1 to 4 carbon atoms, and
Ar represents an aromatic hydrocarbon group having 6 to 20 carbon atoms and optionally having a substituent.

14. The resist composition according to claim 9, comprising a resin including a structural unit represented by a formula (a2-A), 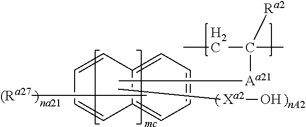
wherein R^{a2} represents a hydrogen atom, a halogen atom, or an alkyl group having 1 to 6 carbon atoms and optionally having a halogen atom,
R^{a27} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group,
A^{a21} represents a single bond or an alkanediyl group having 1 to 12 carbon atoms, and —CH₂— included in the alkanediyl group may be substituted with —O—, —CO— or —NR^{a28}—,
R^{a28} represents a hydrogen atom or an alkyl group having 1 to 6 carbon atoms,
X^{a2} represents a single bond or a carbonyl group,
nA2 represents an integer of 1 to 5, and when nA2 is 2 or more, a plurality of groups in parentheses may be same or different from each other,
na21 represents an integer of 0 to 4, and when na21 is 2 or more, a plurality of R^{a27} may be same or different from each other, and
mc represents an integer of 0 to 2.

15. The resist composition according to claim 9, further comprising a salt generating an acid having an acidity lower than that of an acid generated from the acid generator.

16. A method for producing a resist pattern, the method comprising:
(1) applying the resist composition according to claim 9 on a substrate;
(2) drying the applied composition to form a composition layer;
(3) exposing the composition layer;
(4) heating the exposed composition layer; and
(5) developing the heated composition layer.

17. A salt represented by a formula (I), 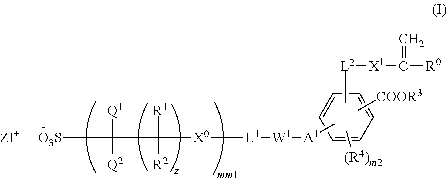
wherein Q¹, Q², R¹, and R² each independently represent a hydrogen atom, a fluorine atom, a perfluoroalkyl group having 1 to 6 carbon atoms, or an alkyl group having 1 to 6 carbon atoms,
z represents an integer of 0 to 6, and when z is 2 or more, a plurality of R¹ and R² may be same or different from each other,
X⁰ represents a single bond, or a group formed by —O—, —CO—, —S—, —SO—, —SO₂—, —NR⁷—, or a combination thereof,
mm1 represents 0 or 1,
L¹ and L² each independently represent a single bond or a hydrocarbon group having 1 to 28 carbon atoms and optionally having a substituent, and —CH₂— included in the hydrocarbon group may be substituted with —O—, —S—, —SO₂—, —SO—, —NR⁷—, or —CO—,
R⁷ represents a hydrogen atom or an alkyl group having 1 to 6 carbon atoms,
W¹ represents a single bond or a cyclic hydrocarbon group having 3 to 36 carbon atoms and optionally having a substituent, and —CH₂— included in the cyclic hydrocarbon group may be substituted with —O—, —S—, —SO₂—, —SO—, —NR⁷—, or —CO—,
A¹ represents a single bond or a hydrocarbon group having 1 to 20 carbon atoms, the hydrocarbon group may have a substituent, and —CH₂— included in the hydrocarbon group may be substituted with —O—, —S—, —SO₂—, —SO—, —NR⁷—, or —CO—,
R³ represents an acid stable hydrocarbon group optionally having a substituent,
R⁴ represents a halogen atom, a cyano group, a nitro group, or a hydrocarbon group having 1 to 18 carbon atoms, the hydrocarbon group may have a substituent, and —CH₂— included in the hydrocarbon group may be substituted with —O—, —S—, —SO₂—, —SO—, —NR⁷—, or —CO—,
m2 represents an integer of 0 to 3, and when m2 is 2 or more, a plurality of R⁴ may be same or different from each other,
X¹ represents a single bond, or —O—, —CO—, —S—, —SO—, —SO₂—, —NR⁷—, a phenylene group optionally having a substituent, or a group obtained by combining these groups,
R⁰ represents a hydrogen atom, a halogen atom, or an alkyl group having 1 to 6 carbon atoms and optionally having a halogen atom, and
ZI⁺ represents an organic cation.

18. The salt according to claim 17, wherein
A¹ is a single bond, ***-X⁰¹-L⁰¹- or ***-L⁰¹-X⁰¹-,
X⁰¹ represents —O—, —CO—, —S—, —SO—, —NR⁷—, or —SO₂—,
L⁰¹ represents a single bond or a hydrocarbon group having 1 to 18 carbon atoms wherein the hydrocarbon group may have a substituent, and —CH₂— included in the hydrocarbon group may be substituted with —O—, —CO—, —S—, —SO—, —NR⁷—, or —SO₂—, and
*** represents a binding site to W¹.

19. The salt according to claim 18, wherein
X⁰¹ is —O— or —CO—, and
L⁰¹ is a single bond or an alkanediyl group having 1 to 6 carbon atoms wherein —CH₂— included in the alkanediyl group may be substituted with —O— or —CO—.

20. The salt according to claim 17, wherein W¹ is a single bond or an aromatic hydrocarbon group having 6 to 10 carbon atoms and optionally having a substituent.

21. The salt according to claim 17, wherein
an acid stable hydrocarbon group optionally having a substituent of R³ is a group represented by a formula (10a-1) or a group represented by a formula (10a-2), 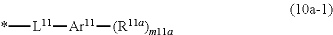
in the formula (10a-1),
L¹¹ represents a single bond or an alkanediyl group having 1 to 6 carbon atoms,
Ar¹¹ represents an aromatic hydrocarbon group having 6 to 18 carbon atoms,
R^{11a} represents a halogen atom or an alkyl group having 1 to 12 carbon atoms and optionally having a halogen atom, and —CH₂— included in the alkyl group may be substituted with —O— or —CO—,
m11a represents an integer of 0 to 8, when m11a is 2 or more, a plurality of R^{11a} may be same or different from each other, and
* represents a binding site, and 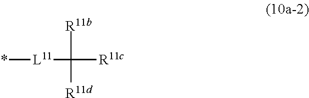
in the formula (10a-2),
L¹¹ represents a single bond or an alkanediyl group having 1 to 6 carbon atoms,
for R^{11b}, R^{11c}, and R^{11d},
R^{11b} and R^{11c} each independently represent a hydrogen atom or a hydrocarbon group having 1 to 18 carbon atoms, and R^{11d} is a hydrogen atom, or
R^{11b} and R^{11c} are bonded to each other to form an alicyclic hydrocarbon group having 3 to 36 carbon atoms and optionally having a halogen atom or optionally having an alkyl group having 1 to 12 carbon atoms and optionally having a halogen atom wherein —CH₂— included in the alkyl group may be substituted with —O— or —CO—, and R^{11d} is a hydrogen atom or
R^{11b}, R^{11c}, and R^{11d} are bonded to each other to form an alicyclic hydrocarbon group having 3 to 36 carbon atoms and optionally having a halogen atom or optionally having an alkyl group having 1 to 12 carbon atoms and optionally having a halogen atom wherein —CH₂— included in the alkyl group may be substituted with —O— or —CO—, and
* represents a binding site.

22. The salt according to claim 17, wherein an acid stable hydrocarbon group optionally having a substituent of R³ is a group including a cyclic hydrocarbon group having 3 to 18 carbon atoms and having at least one selected from the group consisting of a halogen atom and a haloalkyl group having 1 to 12 carbon atoms wherein the cyclic hydrocarbon group may have a substituent other than a halogen atom and a haloalkyl group.

23. The salt according to claim 17, wherein m2 is an integer of 1 or more, and R⁴ is at least one selected from the group consisting of a halogen atom and a hydrocarbon group having 1 to 18 carbon atoms wherein —CH₂— included in the hydrocarbon group may be substituted with —O— or —CO—.

24. The salt according to claim 17, wherein X¹ is a single bond or a group represented by any one of a formula (X¹-1) to a formula (X¹-10), 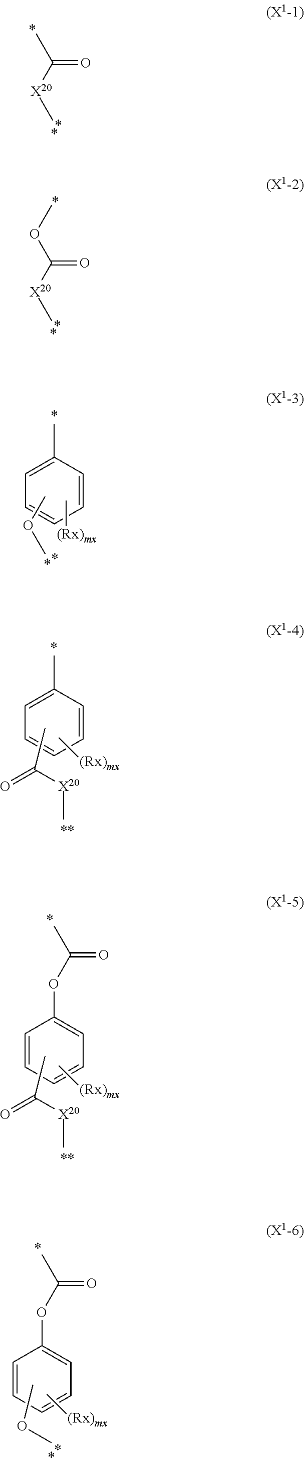 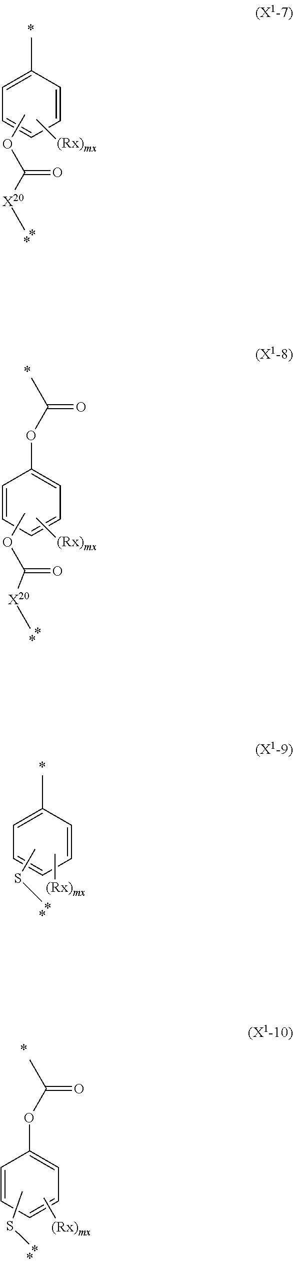
in the formula (X¹-1) to the formula (X¹-10),
* and ** are binding sites, and * represents a binding site to carbon atoms to which R⁰ is bonded,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms,
mx represents an integer of 0 to 4, when mx is 2 or more, a plurality of Rx may be same or different from each other,
X²⁰ represents —O— or —NR⁷—, and
R⁷ represents a hydrogen atom or an alkyl group having 1 to 6 carbon atoms.

25. A resin comprising a structural unit represented by a formula (IP), wherein 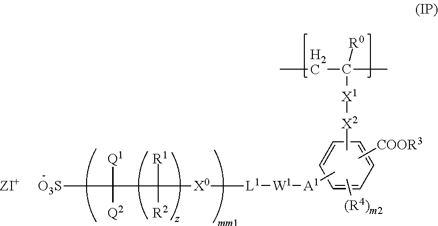
Q¹, Q², R¹, and R² each independently represent a hydrogen atom, a fluorine atom, a perfluoroalkyl group having 1 to 6 carbon atoms, or an alkyl group having 1 to 6 carbon atoms,
z represents an integer of 0 to 6, and when z is 2 or more, a plurality of R¹ and R² may be same or different from each other,
X⁰ represents a single bond, or a group formed by —O—, —CO—, —S—, —SO—, —SO₂—, —NR⁷—, or a combination thereof,
mm1 represents 0 or 1,
L¹ and L² each independently represent a single bond or a hydrocarbon group having 1 to 28 carbon atoms and optionally having a substituent, and —CH₂— included in the hydrocarbon group may be substituted with —O—, —S—, —SO₂—, —SO—, —NR⁷—, or —CO—,
R⁷ represents a hydrogen atom or an alkyl group having 1 to 6 carbon atoms,
W¹ represents a single bond or a cyclic hydrocarbon group having 3 to 36 carbon atoms and optionally having a substituent, and —CH₂— included in the cyclic hydrocarbon group may be substituted with —O—, —S—, —SO₂—, —SO—, —NR⁷—, or —CO—,
A¹ represents a single bond or a hydrocarbon group having 1 to 20 carbon atoms, the hydrocarbon group may have a substituent, and —CH₂— included in the hydrocarbon group may be substituted with —O—, —S—, —SO₂—, —SO—, —NR⁷—, or —CO—,
R³ represents an acid stable hydrocarbon group optionally having a substituent,
R⁴ represents a halogen atom, a cyano group, a nitro group, or a hydrocarbon group having 1 to 18 carbon atoms, the hydrocarbon group may have a substituent, and —CH₂— included in the hydrocarbon group may be substituted with —O—, —S—, —SO₂—, —SO—, —NR⁷—, or —CO—,
m2 represents an integer of 0 to 3, and when m2 is 2 or more, a plurality of R⁴ may be same or different from each other,
X¹ represents a single bond, or —O—, —CO—, —S—, —SO—, —SO₂—, —NR⁷—, a phenylene group optionally having a substituent, or a group obtained by combining these groups,
R⁰ represents a hydrogen atom, a halogen atom, or an alkyl group having 1 to 6 carbon atoms and optionally having a halogen atom, and
ZI⁺ represents an organic cation.
